(19) United States
(12) Patent Application Publication
(10) Pub. No.: US 2024/0384273 A1
(43) Pub. Date: Nov. 21, 2024

(54) HSD17B13-RELATED DOUBLE STRANDED OLIGONUCLEOTIDE COMPOSITIONS AND METHODS RELATING THERETO

(71) Applicant: Wave Life Sciences Ltd., Singapore (SG)

(72) Inventors: Chandra Vargeese, Schwenksville, PA (US); Naoki Iwamoto, Boston, MA (US); Wei Liu, Waltham, MA (US); Mugdha Bedekar, Cambridge, MA (US); Brett Schrand, Reading, MA (US); Priyank Shiva Prakasha, Cambridge, MA (US); Anthony Lamattina, Amherst, NH (US); Luciano H. Apponi, Lynn, MA (US)

(73) Assignee: Wave Life Sciences Ltd., Singapore (SG)

(21) Appl. No.: 18/667,824

(22) Filed: May 17, 2024

Related U.S. Application Data
(63) Continuation of application No. PCT/US2022/050371, filed on Nov. 18, 2022.
(60) Provisional application No. 63/377,482, filed on Sep. 28, 2022.
(60) Provisional application No. 63/268,775, filed on Mar. 2, 2022.
(60) Provisional application No. 63/264,360, filed on Nov. 19, 2021.

(51) Int. Cl. C12N 15/113

(52) U.S. Cl. CPC C12N 15/1137; C12N 2310/14; C12N 2310/315; C12Y 101/01062

(57) ABSTRACT

The present disclosure provides HSD17B13-related double stranded oligonucleotides, compositions, and methods of using such double stranded oligonucleotides and compositions for preventing and/or treating various conditions, disorders, or diseases associated with expression of HSD17B13. In some embodiments, the provided double stranded oligonucleotides and compositions comprise nucleobase modifications, sugar modifications, internucleotidic linkage modifications and/or patterns thereof, and have improved properties, activities and/or selectivities. In some embodiments, the provided double stranded oligonucleotides and compositions target HSD17B13.

### CROSS-REFERENCE TO RELATED APPLICATIONS

This application is a continuation of International Application No. PCT/US2022/050371 filed Nov. 18, 2022, which claims priority to U.S. Provisional Application No. 63/264,360, filed Nov. 19, 2021, U.S. Provisional Application No. 63/268,775, filed Mar. 2, 2022, and U.S. Provisional Application No. 63/377,482, filed Sep. 28, 2022, the contents of each of which are incorporated by reference in their entirety, and to which priority is claimed.

### SEQUENCE LISTING

The present application contains a Sequence Listing which has been submitted via EFS-Web and is hereby incorporated by reference in its entirety. Said Sequence Listing, created on May 17, 2024, is named 0882900165.xml and is 12,751,366 bytes in size.

### TECHNICAL FIELD

Among other things, the present disclosure provides double stranded (ds) oligonucleotides, compositions and methods (e.g., of preparation, use, etc.) thereof. In some embodiments, provided technologies are useful for preventing and/or treating various conditions, disorders, or diseases associated with hydroxysteroid 17-beta dehydrogenase 13 (HSD17B13) expression.

### BACKGROUND

Double stranded (ds) oligonucleotides are useful in various applications, e.g., therapeutic, diagnostic, and/or research applications. For example, ds oligonucleotides targeting HSD17B13 can be useful for treatment of conditions, disorders, or diseases associated with expression of HSD17B13, e.g., nonalcoholic fatty liver disease (NAFLD), nonalcoholic steatohepatitis (NASH), alcoholic steatohepatitis (ASH), alcoholic liver disease, nonalcoholic liver disease, alcoholic cirrhosis, nonalcoholic cirrhosis, steatohepatitis, hepatic steatosis, hepatoceullar carcinoma, HCV hepatitis, chronic hepatitis, hereditary hemochromatosis, primary sclerosing cholangitis, drug induced liver injury, or hepatocellular necrosis.

### SUMMARY

In some embodiments, the present disclosure provides ds oligonucleotides targeting HSD17B13 and compositions thereof that have significantly improved properties and/or high activities. Among other things, the present disclosure provides technologies for designing, manufacturing and utilizing such ds oligonucleotides and compositions. Particularly, in some embodiments, the present disclosure provides ds oligonucleotides comprising useful patterns of internucleotidic linkages and/or patterns of sugar modifications, which, when combined with one or more other structural elements, e.g., base sequence (or portion thereof), nucleobase modifications (and patterns thereof), additional chemical moieties, etc., can provide ds oligonucleotides targeting HSD17B13 and compositions thereof with high activities and/or desired properties, including but not limited to effective and efficient reduction of expression, levels and/or activities of HSD17B13 transcripts and products encoded thereby. In some embodiments, ds oligonucleotides targeting HSD17B13 and compositions reduce levels of a HSD17B13 transcript, and are useful for treating and/or preventing HSD17B13-associated condition, disorder, or disease, e.g., NAFLD, NASH, ASH, alcoholic liver disease, nonalcoholic liver disease, alcoholic cirrhosis, nonalcoholic cirrhosis, steatohepatitis, hepatic steatosis, hepatoceullar carcinoma, HCV hepatitis, chronic hepatitis, hereditary hemochromatosis, primary sclerosing cholangitis, drug induced liver injury, or hepatocellular necrosis.

In some embodiments, a ds oligonucleotide targeting HSD17B13 is capable of mediating knockdown of HSD17B13, wherein the level, expression and/or activity of HSD17B13 or a product thereof are decreased. In some embodiments, a ds oligonucleotide targeting HSD17B13 is capable of mediating pan-specific knockdown of HSD17B13, wherein the level, expression and/or activity of multiple or all HSD17B13 alleles are decreased. In some embodiments, a ds oligonucleotide targeting HSD17B13 has a base sequence that is complementary to a sequence which is common in multiple or all HSD17B13 alleles. In certain embodiments, such structural elements include one or more of: (1) chemical modifications (e.g., modifications of a sugar, base and/or internucleotidic linkage) and patterns thereof; and (2) alterations in stereochemistry (e.g., stereochemistry of a backbone chiral internucleotidic linkage) and patterns thereof. One or more of such structural elements can, in certain embodiments, be independently present in one or both oligonucleotides of a ds oligonucleotide. In certain embodiments, the properties and/or activities impacted by such structural elements include, but are not limited to, participation in, direction of a decrease in expression, activity or level of a gene or a gene product thereof, mediated, for example, by RNA interference (RNAi interference).

In certain embodiments, the present disclosure demonstrates that compositions comprising ds oligonucleotides (e.g., dsRNAi oligonucleotides, also referred to as dsRNAi agents) with controlled structural elements provide unexpected properties and/or activities.

In certain embodiments, the present disclosure encompasses the recognition that stereochemistry, e.g., stereochemistry of backbone chiral centers, can unexpectedly maintain or improve properties of ds oligonucleotides. For example, but not by way of limitation, the instant disclosure relates, in part, to ds oligonucleotides comprising one or more of:
(1) a guide strand comprising backbone phosphorothioate chiral centers in Sp configuration between the 3′ terminal nucleotide and the penultimate (N-1) nucleotide and as between the penultimate (N-1) nucleotide and the immediately upstream, i.e., in the 5′ direction, (N-2) nucleotide;
(2) a guide strand comprising backbone phosphorothioate chiral centers in Rp, Sp, or alternating configurations between the 5′ terminal (+1) nucleotide and the immediately downstream, i.e., in the 3′ direction, (+2) nucleotide and between the +2 nucleotide and the immediately downstream (+3) nucleotide;
(3) a guide strand comprising one or more backbone phosphorothioate chiral centers upstream, i.e., in the 5′ direction, relative to backbone phosphorothioate chiral centers in Sp configuration between the 3′ terminal nucleotide and the penultimate (N-1) nucleotide and as between the penultimate (N-1) nucleotide and the immediately upstream (N-2) nucleotide, where the upstream backbone phosphorothioate chiral centers are in Rp or Sp configuration;
(4) a guide strand comprising one or more backbone phosphorothioate chiral centers in Rp or Sp configuration between the 5′ terminal (+1) nucleotide and the immediately downstream (+2) nucleotide and between the +2 nucleotide and the immediately downstream (+3) nucleotide, as well as between one or both of: (a) the +3 nucleotide and the +4 nucleotide; and (b) the +5 nucleotide and the +6 nucleotide;
(5) a passenger strand in combination with one or more of the aforementioned guide strands, comprising one or more backbone chiral centers in Rp or Sp configuration; and
(6) a passenger strand in combination with one or more of the aforementioned guide strands, comprising backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N-1) nucleotide;
   wherein the ds oligonucleotide further comprises one or more of:
(1) a guide strand where one or both of the 5′ and 3′ terminal dinucleotides are not linked by a Rp, Sp, or stereorandom non-negatively charged internucleotidic linkages, i.e., the guide strand comprises one or more Rp, Sp, or stereorandom non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by a Rp, Sp, or stereorandom non-negatively charged internucleotidic linkage. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain embodiments, the present disclosure encompasses the recognition that stereochemistry, e.g., stereochemistry of chiral centers at a 5′ terminal modification of guide strands, can unexpectedly maintain or improve properties of the ds oligonucleotides described herein. For example, but not by way of limitation, the instant disclosure relates, in part, to ds oligonucleotides comprising a guide stranding comprising: (1) a phosphorothioate chiral center in Rp or Sp configuration; (2) an Rp, Sp, or stereorandom non-negatively charged internucleotidic linkage where the 3′ nucleotide of a nucleotide pair linked by an Rp, Sp, or stereorandom non-negatively charged internucleotidic linkage comprises a 2′ modification, e.g., a 2′ F; and (3) a 5′ terminal modification selected from: a
(a) 5′ PO modifications, such as, but not limited to:

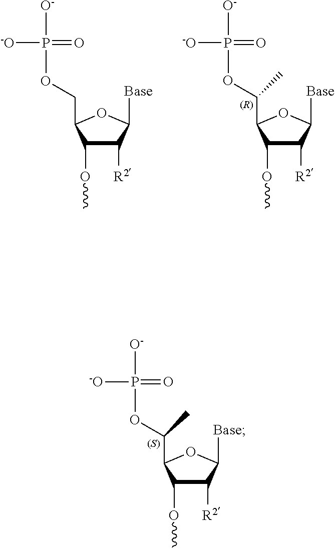

(b) 5′ VP modifications, such as, but not limited to:

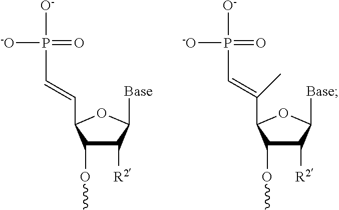

(c) 5′ MeP modifications, such as, but not limited to:

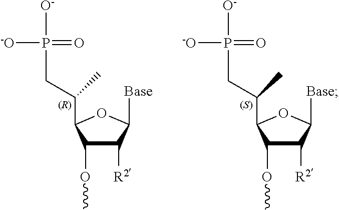

(d) 5′ PN and 5′ Trizole-P modifications, such as, but not limited to:

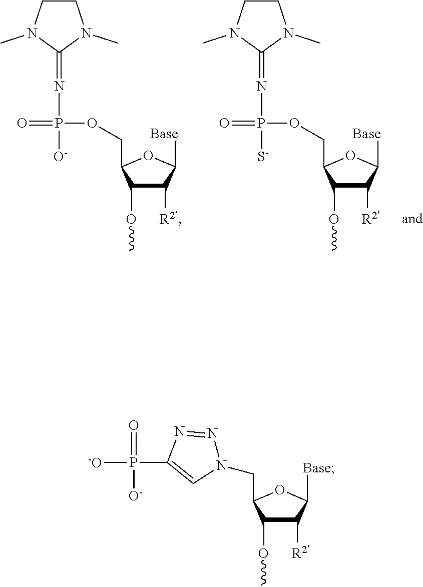

wherein Base is selected from A, C, G, T, U, abasic and modified nucleobases;
R^{2′} is selected from H, OH, O-alkyl, F, MOE, locked nucleic acid (LNA) bridges and bridged nucleic acid (BNA) bridges to the 4′ C, such as, but not limited to:

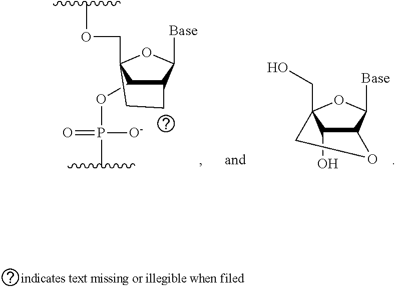

In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain other embodiments, the present disclosure encompasses the recognition that stereochemistry, e.g., stereochemistry of chiral centers at the 5′ terminal nucleotide of guide strands, can unexpectedly maintain or improve properties of ds oligonucleotides wherein the guide strand of the ds oligonucleotide also comprises a phosphorothioate chiral center in Rp or Sp configuration. For example, but not by way of limitation, the instant disclosure relates, in part, to ds oligonucleotides comprising a guide stranding comprising: (1) a phosphorothioate chiral center in Rp or Sp configuration; (2) an Rp, Sp, or stereorandom non-negatively charged internucleotidic linkage where the 3′ nucleotide of a nucleotide pair linked by an Rp, Sp, or stereorandom non-negatively charged internucleotidic linkage comprises a 2′ modification, e.g., a 2′ F; and (3) a 5′ terminal modification selected from:
(a) 5′ PO nucleotides, such as, but not limited to:

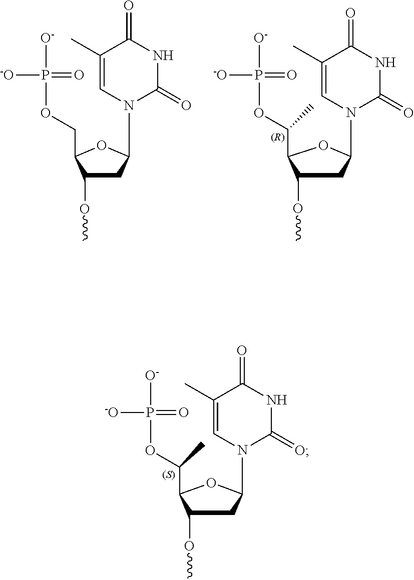

(b) 5′ VP nucleotides, such as, but not limited to:

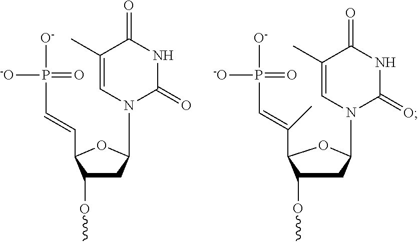

(c) 5′ MeP nucleotides, such as, but not limited to:

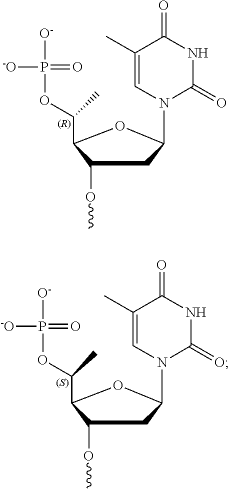

(d) 5′ PN and 5′ Trizole-P nucleotides, such as, but not limited to:

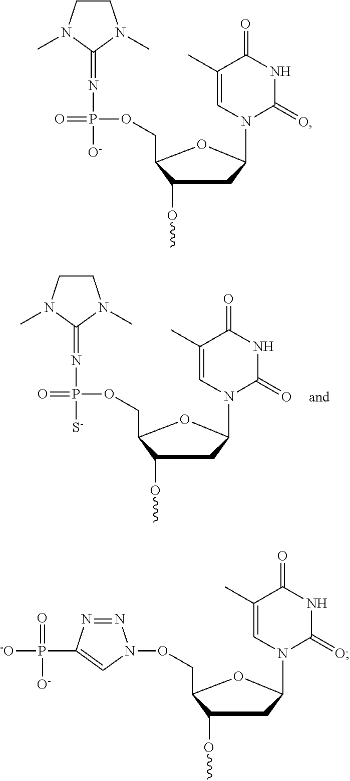

(e) 5′ abasic VP and 5′ abasic MeP nucleotides, such as, but not limited to:

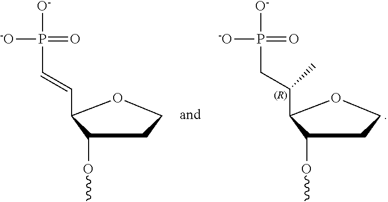

In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain embodiments, the present disclosure encompasses the recognition that non-naturally occurring internucleotidic linkages, e.g., neutral internucleotidic linkages, can, in certain embodiments, be used to link one or more molecules to the double-stranded oligonucleotides described herein. In certain embodiments, such linked molecules can facilitate targeting and/or delivery of the double-stranded oligonucleotide. For example, but not limitation, such linked molecules an include lipophilic molecules. In certain embodiments, the linked molecule is a molecule comprising one or more GalNAc moieties. In certain embodiments, the linked molecule is a receptor. In certain embodiments, the linked molecule is a receptor ligand.

In certain embodiments, the present disclosure provides technologies for incorporating various additional chemical moieties into ds oligonucleotides. In certain embodiments, the present disclosure provides, for example, reagents and methods for introducing additional chemical moieties through nucleobases (e.g., by covalent linkage, optionally via a linker, to a site on a nucleobase).

In certain embodiments, the present disclosure provides technologies, e.g., ds oligonucleotide compositions and methods thereof, that achieve allele-specific suppression, wherein transcripts from one allele of a particular target gene is selectively knocked down relative to at least one other allele of the same gene.

Among other things, the present disclosure provides structural elements, technologies and/or features that can be incorporated into ds oligonucleotides and can impart or tune one or more properties thereof (e.g., relative to an otherwise identical ds oligonucleotide lacking the relevant technology or feature). In certain embodiments, the present disclosure documents that one or more provided technologies and/or features can usefully be incorporated into ds oligonucleotides of various sequences.

In certain embodiments, the present disclosure demonstrates that certain provided structural elements, technologies and/or features are particularly useful for ds oligonucleotides that participate in and/or direct RNAi mechanisms (e.g., RNAi agents). Regardless, however, the teachings of the present disclosure are not limited to ds oligonucleotides that participate in or operate via any particular mechanism. In certain embodiments, the present disclosure pertains to any ds oligonucleotide, useful for any purpose, which operates through any mechanism, and which comprises any sequence, structure or format (or portion thereof) described herein. In certain embodiments, the present disclosure provides a ds oligonucleotide, useful for any purpose, which operates through any mechanism, and which comprises any sequence, structure or format (or portion thereof) described herein, comprising one or more of:
(1) a guide strand comprising backbone phosphorothioate chiral centers in Sp configuration between the 3′ terminal nucleotide and the penultimate (N-1) nucleotide and as between the penultimate (N-1) nucleotide and the immediately upstream, i.e., in the 5′ direction, (N-2) nucleotide;
(2) a guide strand comprising backbone phosphorothioate chiral centers in Rp, Sp, or alternating configurations between the 5′ terminal (+1) nucleotide and the immediately downstream, i.e., in the 3′ direction, (+2) nucleotide and between the +2 nucleotide and the immediately downstream (+3) nucleotide;
(3) a guide strand comprising one or more backbone phosphorothioate chiral centers upstream, i.e., in the 5′ direction, relative to backbone phosphorothioate chiral centers in Sp configuration between the 3′ terminal nucleotide and the penultimate (N-1) nucleotide and as between the penultimate (N-1) nucleotide and the immediately upstream (N-2) nucleotide, where the upstream backbone phosphorothioate chiral centers are in Rp or Sp configuration;
(4) a guide strand comprising one or more backbone phosphorothioate chiral centers in Rp or Sp configuration between the 5′ terminal (+1) nucleotide and the immediately downstream (+2) nucleotide and between the +2 nucleotide and the immediately downstream (+3) nucleotide, as well as between one or both of: (a) the +3 nucleotide and the +4 nucleotide; and (b) the +5 nucleotide and the +6 nucleotide;
(5) a passenger strand in combination with one or more of the aforementioned guide strands, comprising one or more backbone chiral centers in Rp or Sp configuration; and
6) a passenger strand in combination with one or more of the aforementioned guide strands, comprising backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N-1) nucleotide;
   wherein the ds oligonucleotide further comprises one or more of:
(1) a guide strand where one or both of the 5′ and 3′ terminal dinucleotides are not linked by non-negatively charged internucleotidic linkages, i.e., the guide strand comprises one more Rp, Sp, or stereorandom non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain embodiments, the provided ds oligonucleotides may participate in (e.g., direct) RNAi mechanisms.

In certain embodiments, the guide strand comprises backbone phosphorothioate chiral centers in Sp configuration between the 3′ terminal nucleotide and the penultimate (N-1) nucleotide and as between the penultimate (N-1) nucleotide and the immediately upstream (N-2) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49,

In certain embodiments, the present disclosure demonstrates that compositions comprising ds oligonucleotides (e.g., dsRNAi oligonucleotides, also referred to as dsRNAi agents) with controlled structural elements provide unexpected properties and/or activities.

In certain embodiments, the guide strand comprises backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain embodiments, the guide strand comprises one or more backbone phosphorothioate chiral centers in Rp or Sp configuration upstream of backbone phosphorothioate chiral centers in Sp configuration between the 3′ terminal nucleotide and the penultimate (N-1) nucleotide and as between the penultimate (N-1) nucleotide and the immediately upstream (N-2) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain embodiments, the guide strand comprises one or more Rp, Sp, or stereorandom non-negatively charged internucleotidic linkage occurs between the second (+2) and third (+3) nucleotides, relative to the 5′ terminal nucleotide, of the guide strand and the internucleotidic linkage to the penultimate 3′ (N-1) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain embodiments, the guide strand comprises backbone phosphorothioate chiral centers in Sp configuration between the 3′ terminal nucleotide and the penultimate (N-1) nucleotide and as between the penultimate (N-1) nucleotide and the immediately upstream (N-2) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone phosphorothioate chiral centers in Rp or Sp configuration. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain embodiments, the guide strand comprises backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone chiral centers in Rp or Sp configuration. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain embodiments, the guide strand comprises one or more backbone phosphorothioate chiral centers in Rp or Sp configuration upstream of backbone chiral centers in Sp configuration between the 3′ terminal nucleotide and the penultimate (N-1) nucleotide and as between the penultimate (N-1) nucleotide and the immediately upstream (N-2) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone chiral centers in Rp or Sp configuration. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain embodiments, the guide strand comprises one or more backbone phosphorothioate chiral centers in Rp or Sp configuration between the 5′ terminal (+1) nucleotide and the immediately downstream (+2) nucleotide and between the (+2) nucleotide and the immediately downstream (+3) nucleotide, as well as between one or both of: (a) the (+3) nucleotide and the (+4) nucleotide; and (b) the (+5) nucleotide and the (+6) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone chiral centers in Rp or Sp configuration. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain embodiments, the guide strand comprises one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone chiral centers in Rp or Sp configuration. In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain embodiments, the guide strand comprises backbone phosphorothioate chiral centers in Sp configuration between the 3′ terminal nucleotide and the penultimate (N-1) nucleotide and as between the penultimate (N-1) nucleotide and the immediately upstream (N-2) nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49 and one or more backbone chiral centers in Rp or Sp configuration. In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain embodiments, the guide strand comprises backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49 and one or more backbone chiral centers in Rp or Sp configuration. In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain embodiments, the guide strand comprises one or more backbone phosphorothioate chiral centers in Rp or Sp configuration upstream of backbone phosphorothioate chiral centers in Sp configuration between the 3′ terminal nucleotide and the penultimate (N-1) nucleotide and as between the penultimate (N-1) nucleotide and the immediately upstream (N-2) nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49 and one or more backbone chiral centers in Rp or Sp configuration. In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain embodiments, the guide strand comprises one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49 and one or more backbone chiral centers in Rp or Sp configuration. In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain embodiments, provided ds oligonucleotides may participate in exon skipping mechanisms. In certain embodiments, provided ds oligonucleotides may be aptamers. In certain embodiments, provided ds oligonucleotides may bind to and inhibit the function of a protein, small molecule, nucleic acid or cell. In certain embodiments, provided ds oligonucleotides may participate in forming a triplex helix with a double-stranded nucleic acid in the cell. In certain embodiments, provided ds oligonucleotides may bind to genomic (e.g., chromosomal) nucleic acid. In certain embodiments, provided ds oligonucleotides may bind to genomic (e.g., chromosomal) nucleic acid, thus preventing or decreasing expression of the nucleic acid (e.g., by preventing or decreasing transcription, transcriptional enhancement, modification, etc.). In certain embodiments, provided ds oligonucleotides may bind to DNA quadruplexes. In certain embodiments, provided ds oligonucleotides may be immunomodulatory. In certain embodiments, provided ds oligonucleotides may be immunostimulatory. In certain embodiments, provided oligonucleotides may be immunostimulatory and may comprise a CpG sequence. In certain embodiments, provided ds oligonucleotides may be immunostimulatory and may comprise a CpG sequence and may be useful as an adjuvant. In certain embodiments, provided ds oligonucleotides may be immunostimulatory and may comprise a CpG sequence and may be useful as an adjuvant in treating a disease (e.g., an infectious disease or cancer). In certain embodiments, provided ds oligonucleotides may be therapeutic. In certain embodiments, provided ds oligonucleotides may be non-therapeutic. In certain embodiments, provided ds oligonucleotides may be therapeutic or non-therapeutic. In certain embodiments, provided ds oligonucleotides are useful in therapeutic, diagnostic, research and/or nanomaterials applications. In certain embodiments, provided ds oligonucleotides may be useful for experimental purposes. In certain embodiments, provided ds oligonucleotides may be useful for experimental purposes, e.g., as a probe, in a microarray, etc. In certain embodiments, provided ds oligonucleotides may participate in more than one biological mechanism; in certain such embodiments, for example, provided ds oligonucleotides may participate in both RNAi and RNase H mechanisms.

In certain embodiments, provided ds oligonucleotides are directed to an HSD17B13 target (e.g., an HSD17B13 target sequence, an HSD17B13 target RNA, an HSD17B13 target mRNA, an HSD17B13 target pre-mRNA, an HSD17B13 target gene, etc.). An HSD17B13 target gene is a gene with respect to which expression and/or activity of one or more HSD17B13 gene products (e.g., HSD17B13 RNA and/or protein products) are intended to be altered. In certain embodiments, an HSD17B13 target gene is intended to be inhibited. Thus, when a ds oligonucleotide as described herein acts on an HSD17B13 target gene, presence and/or activity of one or more HSD17B13 gene products are altered when the ds oligonucleotide is present as compared with when it is absent.

In certain embodiments, an HSD17B13 target is a specific HSD17B13 allele with respect to which expression and/or activity of one or more products (e.g., HSD17B13 RNA and/or protein products) are intended to be altered. In certain embodiments, an HSD17B13 target allele is one whose presence and/or expression is associated (e.g., correlated) with presence, incidence, and/or severity, of one or more HSD17B13 associated diseases and/or conditions. Alternatively or additionally, in certain embodiments, an HSD17B13 target allele is one for which alteration of level and/or activity of one or more HSD17B13 gene products correlates with improvement (e.g., delay of onset, reduction of severity, responsiveness to other therapy, etc) in one or more aspects of an HSD17B13 associated disease and/or condition.

In certain embodiments, e.g., where presence and/or activity of a particular HSD17B13 allele (an HSD17B13 disease-associated allele) is associated (e.g., correlated) with presence, incidence and/or severity of one or more disorders, diseases and/or conditions, a different HSD17B13 allele exists and is not so associated, or is associated to a lesser extent (e.g., shows less significant, or statistically insignificant correlation), ds oligonucleotides and methods thereof as described herein may preferentially or specifically target the associated allele relative to the one or more less-associated/unassociated allele(s), thus mediating allele-specific suppression.

In certain embodiments, an HSD17B13 target sequence is an HSD17B13 sequence to which an oligonucleotide as described herein binds. In certain embodiments, an HSD17B13 target sequence is identical to, or is an exact complement of, an HSD17B13 sequence of a provided oligonucleotide, or of consecutive residues therein (e.g., a provided oligonucleotide includes an HSD17B13 target-binding sequence that is identical to, or an exact complement of, an HSD17B13 target sequence). In certain embodiments, an HSD17B13 target-binding sequence is an exact complement of an HSD17B13 target sequence of an HSD17B13 transcript (e.g., pre-mRNA, mRNA, etc.). An HSD17B13 target-binding sequence/target sequence can be of various lengths to provided oligonucleotides with desired activities and/or properties. In certain embodiments, an HSD17B13 target binding sequence/target sequence comprises 5-50 (e.g., 10-40, 15-30, 15-25, 16-25, 17-25, 18-25, 19-25, 20-25, 10, 11, 12, 13, 14, 15, 16, 17, 18, 19, 20, 21, 22, 23, 24, 25, or more) bases. In certain embodiments, a small number of differences/mismatches is tolerated between (a relevant portion of) an oligonucleotide and its target sequence, including but not limited to the 5′ and/or 3′-end regions of the HSD17B13 target and/or oligonucleotide sequence. In certain embodiments, an HSD17B13 target sequence is present within an HSD17B13 target gene. In certain embodiments, an HSD17B13 target sequence is present within an HSD17B13 transcript (e.g., an mRNA and/or a pre-mRNA) produced from an HSD17B13 target gene.

In certain embodiments, an HSD17B13 target sequence includes one or more allelic sites (i.e., positions within an HSD17B13 target gene at which allelic variation occurs). In certain embodiments, an allelic site is a mutation. In certain embodiments, an allelic site is a SNP. In some such embodiments, a provided oligonucleotide binds to one allele preferentially or specifically relative to one or more other alleles. In certain embodiments, a provided oligonucleotide binds preferentially to a disease-associated allele. For example, in certain embodiments, an oligonucleotide (or a target-binding sequence portion thereof) provided herein has a sequence that is, fully or at least in part, identical to, or an exact complement of a particular allelic version of an HSD17B13 target sequence.

In certain embodiments, an oligonucleotide (or a target-binding sequence portion thereof) provided herein has a sequence that is identical to, or an exact complement of an HSD17B13 target sequence comprising an allelic site, or an allelic site, of a disease-associated allele. In certain embodiments, an oligonucleotide provided herein has an HSD17B13 target binding sequence that is an exact complement of an HSD17B13 target sequence comprising an allelic site of an HSD17B13 transcript of an allele (in certain embodiments, a disease-associated allele), wherein the allelic site is a mutation. In certain embodiments, an oligonucleotide provided herein has an HSD17B13 target binding sequence that is an exact complement of an HSD17B13 target sequence comprising an allelic site of an HSD17B13 transcript of an allele (in certain embodiments, a disease-associated allele), wherein the allelic site is a SNP. In certain embodiments, a sequence is any sequence disclosed herein.

Unless otherwise noted, all sequences (including, but not limited to base sequences and patterns of chemistry, modification, and/or stereochemistry) are presented in 5′ to 3′ order, with the 5′ terminal nucleotide identified as the “+1” position and the 3′ terminal nucleotide identified either by the number of nucleotides of the full sequence or by “N”, with the penultimate nucleotide identified, e.g., as “N-1”, and so on.

In certain embodiments, the present disclosure provides compositions and methods related to an oligonucleotide which is specific to an HSD17B13 target and which has any format, structural element or base sequence of any oligonucleotide disclosed herein.

In certain embodiments, the present disclosure provides compositions and methods related to an oligonucleotide which is specific to an HSD17B13 target and which has or comprises the base sequence of any oligonucleotide disclosed herein, or a region of at least 15 contiguous nucleotides of the base sequence of any oligonucleotide disclosed herein, wherein the first nucleotide of the base sequence or the first nucleotide of the at least 15 contiguous nucleotides can be optionally replaced by T or DNA T.

In certain embodiments, the present disclosure provides compositions and methods for RNA interference directed by a RNAi agent (also referred to as a RNAi oligonucleotides). In certain embodiments, oligonucleotides of such compositions can have a format, structural element or base sequence of an oligonucleotide disclosed herein.

In certain embodiments, the present disclosure provides compositions and methods for RNase H-mediated knockdown of an HSD17B13 target gene RNA directed by an oligonucleotide (e.g., an antisense oligonucleotide).

Provided oligonucleotides and oligonucleotide compositions can have any format, structural element or base sequence of any oligonucleotide disclosed herein. In certain embodiments, a structural element is a 5′-end structure, 5′-end region, 5′-nucleotide, seed region, post-seed region, 3′-end region, 3′-terminal dinucleotide, 3′-end cap, or any portion of any of these structures, GC content, long GC stretch, and/or any modification, chemistry, stereochemistry, pattern of modification, chemistry or stereochemistry, or a chemical moiety (e.g., including but not limited to, a targeting moiety, a lipid moiety, a GalNAc moiety, a carbohydrate moiety, etc.), any component, or any combination of any of the above.

In certain embodiments, the present disclosure provides compositions and methods of use of an oligonucleotide.

In certain embodiments, the present disclosure provides compositions and methods of use of an oligonucleotide which can direct both RNA interference and RNase H-mediated knockdown of an HSD17B13 target gene RNA. In certain embodiments, oligonucleotides of such compositions can have a format, structural element or base sequence of an oligonucleotide disclosed herein.

In certain embodiments, an oligonucleotide directing a particular event or activity participates in the particular event or activity, e.g., a decrease in the expression, level or activity of a target gene or a gene product thereof. In certain embodiments, an oligonucleotide is deemed to “direct” a particular event or activity when presence of the oligonucleotide in a system in which the event or activity can occur correlates with increased detectable incidence, frequency, intensity and/or level of the event or activity.

In certain embodiments, a provided oligonucleotide comprises any one or more structural elements of an oligonucleotide as described herein, e.g., a base sequence (or a portion thereof of at least 15 contiguous bases); a pattern of internucleotidic linkages (or a portion thereof of at least 5 contiguous internucleotidic linkage); a pattern of stereochemistry of internucleotidic linkages (or a portion thereof of at least 5 contiguous internucleotidic linkages); a 5′-end structure; a 5′-end region; a first region; a second region; and a 3′-end region (which can be a 3′-terminal dinucleotide and/or a 3′-end cap); and an optional additional chemical moiety; and, in certain embodiments, at least one structural element comprises a chirally controlled chiral center. In certain embodiments, a 3′-terminal dinucleotide can comprise two total nucleotides. In certain embodiments, an oligonucleotide further comprises a chemical moiety selected from, as non-limiting examples, a targeting moiety, a carbohydrate moiety, a GalNAc moiety, a lipid moiety, and any other chemical moiety described herein or known in the art. In certain embodiments, a moiety that binds APGR is a moiety of GalNAc, or a variant, derivative or modified version thereof, as described herein and/or known in the art. In certain embodiments, an oligonucleotide is a RNAi agent. In certain embodiments, a first region is a seed region. In certain embodiments, a second region is a post-seed region.

In certain embodiments, a provided oligonucleotide comprises any one or more structural elements of a RNAi agent as described herein, e.g., a 5′-end structure; a 5′-end region; a seed region; a post-seed region (the region between the seed region and the 3′-end region); and a 3′-end region (which can be a 3′-terminal dinucleotide and/or a 3′-end cap); and an optional additional chemical moiety; and, in certain embodiments, at least one structural element comprises a chirally controlled chiral center. In certain embodiments, a 3′-terminal dinucleotide can comprise two total nucleotides. In certain embodiments, an oligonucleotide further comprises a chemical moiety selected from, as non-limiting examples, a targeting moiety, a carbohydrate moiety, a GalNAc moiety, and a lipid moiety. In certain embodiments, a moiety that binds APGR is any GalNAc, or variant, derivative or modification thereof, as described herein or known in the art.

In certain embodiments, a provided oligonucleotide comprises any one or more structural elements of an oligonucleotide as described herein, e.g., a 5′-end structure, a 5′-end region, a first region, a second region, a 3′-end region, and an optional additional chemical moiety, wherein at least one structural element comprises a chirally controlled chiral center. In certain embodiments, the oligonucleotide comprises a span of at least 5 total nucleotides without 2′-modifications. In certain embodiments, the oligonucleotide further comprises an additional chemical moiety selected from, as non-limiting examples, a targeting moiety, a carbohydrate moiety, a GalNAc moiety, and a lipid moiety. In certain embodiments, a provided oligonucleotide is capable of directing RNA interference. In certain embodiments, a provided oligonucleotide is capable of directing RNase H-mediated knockdown. In certain embodiments, a provided oligonucleotide is capable of directing both RNA interference and RNase H-mediated knockdown. In certain embodiments, a first region is a seed region. In certain embodiments, a second region is a post-seed region.

In certain embodiments, a provided oligonucleotide comprises any one or more structural elements of a RNAi agent, e.g., a 5′-end structure, a 5′-end region, a seed region, a post-seed region, and a 3′-end region and an optional additional chemical moiety, wherein at least one structural element comprises a chirally controlled chiral center; and, in certain embodiments, the oligonucleotide is also capable of directing RNase H-mediated knockdown of a target gene RNA. In certain embodiments, the oligonucleotide comprises a span of at least 5 total 2′-deoxy nucleotides. In certain embodiments, the oligonucleotide further comprises a chemical moiety selected from, as non-limiting examples, a targeting moiety, a carbohydrate moiety, a GalNAc moiety, and a lipid moiety, and any other additional chemical moiety described herein.

In certain embodiments, the present disclosure demonstrates that oligonucleotide properties can be modulated through chemical modifications. In certain embodiments, the present disclosure provides an oligonucleotide composition comprising a first plurality of oligonucleotides which have a common base sequence and comprise one or more internucleotidic linkage, sugar, and/or base modifications. In certain embodiments, the present disclosure provides an oligonucleotide composition capable of directing RNA interference and comprising a first plurality of oligonucleotides which have a common base sequence and comprise one or more internucleotidic linkage, and/or one or more sugar, and/or one or more base modifications. In certain embodiments, an oligonucleotide or oligonucleotide composition is also capable of directing RNase H-mediated knockdown of an HSD17B13 target gene RNA. In certain embodiments, the present disclosure demonstrates that oligonucleotide properties, e.g., activities, toxicities, etc., can be modulated through chemical modifications of sugars, nucleobases, and/or internucleotidic linkages. In certain embodiments, the present disclosure provides an oligonucleotide composition comprising a plurality of oligonucleotides which have a common base sequence, and comprise one or more modified internucleotidic linkages (or “non-natural internucleotidic linkages”, linkages that can be utilized in place of a natural phosphate internucleotidic linkage (—OP(O)(OH)O—, which may exist as a salt form (—OP(O)(O⁻)O—) at a physiological pH) found in natural DNA and RNA), one or more modified sugar moieties, and/or one or more natural phosphate linkages. In certain embodiments, provided oligonucleotides may comprise two or more types of modified internucleotidic linkages. In certain embodiments, a provided oligonucleotide comprises a non-negatively charged internucleotidic linkage. In certain embodiments, a non-negatively charged internucleotidic linkage is a neutral internucleotidic linkage. In certain embodiments, a neutral internucleotidic linkage comprises a cyclic guanidine moiety. Such moieties an optionally substituted. In certain embodiments, a provided oligonucleotide comprises a neutral internucleotidic linkage and another internucleotidic linkage which is not a neutral backbone. In certain embodiments, a provided oligonucleotide comprises a neutral internucleotidic linkage and a phosphorothioate internucleotidic linkage. In certain embodiments, provided oligonucleotide compositions comprising a plurality of oligonucleotides are chirally controlled and level of the plurality of oligonucleotides in the composition is controlled or pre-determined, and oligonucleotides of the plurality share a common stereochemistry configuration at one or more chiral internucleotidic linkages. For example, in certain embodiments, oligonucleotides of a plurality share a common stereochemistry configuration at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 or more chiral internucleotidic linkages, each of which is independently Rp or Sp; in certain embodiments, oligonucleotides of a plurality share a common stereochemistry configuration at each chiral internucleotidic linkages. In certain embodiments, a chiral internucleotidic linkage where a controlled level of oligonucleotides of a composition share a common stereochemistry configuration (independently in the Rp or Sp configuration) is referred to as a chirally controlled internucleotidic linkage. In certain embodiments, a modified internucleotidic linkage is a non-negatively charged (neutral or cationic) internucleotidic linkage in that at a pH, (e.g., human physiological pH (˜7.4), pH of a delivery site (e.g., an organelle, cell, tissue, organ, organism, etc.), etc.), it largely (e.g., at least 5%, 10%, 20%, 30%, 40%, 50%, 60%, 70%, 80%, 90%, etc.; in certain embodiments, at least 30%; in certain embodiments, at least 40%; in certain embodiments, at least 50%; in certain embodiments, at least 60%; in certain embodiments, at least 70%; in certain embodiments, at least 80%; in certain embodiments, at least 90%; in certain embodiments, at least 99%; etc.) exists as a neutral or cationic form (as compared to an anionic form (e.g., —O—P(O)(O⁻)—O— (the anionic form of natural phosphate linkage), —O—P(O)(S⁻)—O— (the anionic form of phosphorothioate linkage), etc.)), respectively. In certain embodiments, a modified internucleotidic linkage is a neutral internucleotidic linkage in that at a pH, it largely exists as a neutral form. In certain embodiments, a modified internucleotidic linkage is a cationic internucleotidic linkage in that at a pH, it largely exists as a cationic form. In certain embodiments, a pH is human physiological pH (˜7.4). In certain embodiments, a modified internucleotidic linkage is a neutral internucleotidic linkage in that at pH 7.4 in a water solution, at least 90% of the internucleotidic linkage exists as its neutral form. In certain embodiments, a modified internucleotidic linkage is a neutral internucleotidic linkage in that in a water solution of the oligonucleotide, at least 50%, 60%, 70%, 80%, 90%, 95%, or 99% of the internucleotidic linkage exists in its neutral form. In certain embodiments, the percentage is at least 90%. In certain embodiments, the percentage is at least 95%. In certain embodiments, the percentage is at least 99%. In certain embodiments, a non-negatively charged internucleotidic linkage, e.g., a neutral internucleotidic linkage, when in its neutral form has no moiety with a pKa that is less than 8, 9, 10, 11. 12, 13, or 14. In certain embodiments, pKa of an internucleotidic linkage in the present disclosure can be represented by pKa of CH₃— the internucleotidic linkage —CH₃ (i.e., replacing the two nucleoside units connected by the internucleotidic linkage with two —CH₃ groups). Without wishing to be bound by any particular theory, in at least some cases, a neutral internucleotidic linkage in an oligonucleotide can provide improved properties and/or activities, e.g., improved delivery, improved resistance to exonucleases and endonucleases, improved cellular uptake, improved endosomal escape and/or improved nuclear uptake, etc., compared to a comparable nucleic acid which does not comprises a neutral internucleotidic linkage.

In certain embodiments, a non-negatively charged internucleotidic linkage has the structure of e.g., of formula I-n-1, I-n-2, I-n-3, II, II-a-1, II-a-2, H-b-1, II-b-2, II-c-1, II-c-2, II-d-1, II-d-2, as described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612 etc. In certain embodiments, a non-negatively charged internucleotidic linkage comprises a cyclic guanidine moiety. In certain embodiments, a modified internucleotidic linkage comprising a cyclic guanidine moiety has the structure of:

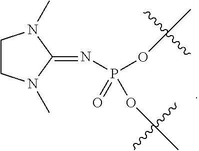 [SMILES: *OP(=O)(N=C1N(C)CCN1C)O*]

In certain embodiments, a neutral internucleotidic linkage comprising a cyclic guanidine moiety is chirally controlled. In certain embodiments, the present disclosure pertains to a composition comprising an oligonucleotide comprising at least one neutral internucleotidic linkage and at least one phosphorothioate internucleotidic linkage.

In certain embodiments, the present disclosure pertains to a composition comprising an oligonucleotide comprising at least one neutral internucleotidic linkage and at least one phosphorothioate internucleotidic linkage, wherein the phosphorothioate internucleotidic linkage is a chirally controlled internucleotidic linkage in the Sp configuration.

In certain embodiments, the present disclosure pertains to a composition comprising an oligonucleotide comprising at least one neutral internucleotidic linkage and at least one phosphorothioate internucleotidic linkage, wherein the phosphorothioate is a chirally controlled internucleotidic linkage in the Rp configuration.

In certain embodiments, the present disclosure pertains to a composition comprising an oligonucleotide comprising at least one neutral internucleotidic linkage of a neutral internucleotidic linkage comprising a Tmg group

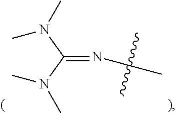 [SMILES: *N=C(N(C)C)N(C)C]

and at least one phosphorothioate.

In certain embodiments, each internucleotidic linkage in an oligonucleotide is independently selected from a natural phosphate linkage, a phosphorothioate linkage, and a non-negatively charged internucleotidic linkage (e.g., n001, n003, n004, n006, n008, n009, n013, n020, n021, n025, n026, n029, n031, n037, n046, n047, n048, n054, or n055). In some embodiments, each internucleotidic linkage in an oligonucleotide is independently selected from a natural phosphate linkage, a phosphorothioate linkage, and a neutral internucleotidic linkage (e.g., n001, n003, n004, n006, n008, n009, n013 n020, n021, n025, n026, n029, n031, n037, n046, n047, n048, n054, or n055).

In certain embodiments, the present disclosure pertains to a composition comprising an oligonucleotide comprising at least one neutral internucleotidic linkage of a neutral internucleotidic linkage comprising a Tmg group, and at least one phosphorothioate, wherein the phosphorothioate is a chirally controlled internucleotidic linkage in the Sp configuration.

In certain embodiments, the present disclosure pertains to a composition comprising an oligonucleotide comprising at least one neutral internucleotidic linkage selected from a neutral internucleotidic linkage of a neutral internucleotidic linkage comprising a Tmg group, and at least one phosphorothioate, wherein the phosphorothioate is a chirally controlled internucleotidic linkage in the Rp configuration.

Various types of internucleotidic linkages differ in properties. Without wishing to be bound by any theory, the present disclosure notes that a natural phosphate linkage (phosphodiester internucleotidic linkage) is anionic and may be unstable when used by itself without other chemical modifications in vivo; a phosphorothioate internucleotidic linkage is anionic, generally more stable in vivo than a natural phosphate linkage, and generally more hydrophobic; a neutral internucleotidic linkage such as one exemplified in the present disclosure comprising a cyclic guanidine moiety is neutral at physiological pH, can be more stable in vivo than a natural phosphate linkage, and more hydrophobic.

In certain embodiments, a chirally controlled neutral internucleotidic linkage sis neutral at physiological pH, chirally controlled, stable in vivo, hydrophobic, and may increase endosomal escape.

In certain embodiments, provided oligonucleotides comprise one or more regions, e.g., a block, wing, core, 5′-end, 3′-end, middle, seed, post-seed region, etc. In certain embodiments, a region (e.g., a block, wing, core, 5′-end, 3′-end, middle region, etc.) comprises a non-negatively charged internucleotidic linkage, e.g., of formula I-n-1, I-n-2, I-n-3, II, II-a-1, II-a-2, II-b-1, II-b-2, II-c-1, II-c-2, II-d-1, II-d-2, etc as described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612. In certain embodiments, a region comprises a neutral internucleotidic linkage. In certain embodiments, a region comprises an internucleotidic linkage which comprises a cyclic guanidine guanidine. In certain embodiments, a region comprises an internucleotidic linkage which comprises a cyclic guanidine moiety. In certain embodiments, a region comprises an internucleotidic linkage having the structure of

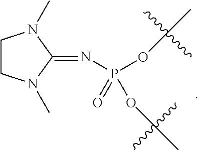 [SMILES: *OP(=O)(N=C1N(C)CCN1C)O*]

In certain embodiments, such internucleotidic linkages are chirally controlled.

In certain embodiments, a nucleotide is a natural nucleotide. In certain embodiments, a nucleotide is a modified nucleotide. In certain embodiments, a nucleotide is a nucleotide analog. In certain embodiments, a base is a modified base. In certain embodiments, a base is protected nucleobase, such as a protected nucleobase used in oligonucleotide synthesis. In certain embodiments, a base is a base analog. In certain embodiments, a sugar is a modified sugar. In certain embodiments, a sugar is a sugar analog. In certain embodiments, an internucleotidic linkage is a modified internucleotidic linkage. In certain embodiments, a nucleotide comprises a base, a sugar, and an internucleotidic linkage, wherein each of the base, the sugar, and the internucleotidic linkage is independently and optionally naturally-occurring or non-naturally occurring. In certain embodiments, a nucleoside comprises a base and a sugar, wherein each of the base and the sugar is independently and optionally naturally-occurring or non-naturally occurring. Non-limiting examples of nucleotides include DNA (2′-deoxy) and RNA (2′-OH) nucleotides; and those which comprise one or more modifications at the base, sugar and/or internucleotidic linkage. Non-limiting examples of sugars include ribose and deoxyribose; and ribose and deoxyribose with 2′-modifications, including but not limited to 2′-F, LNA, 2′-OMe, and 2′-MOE modifications. In certain embodiments, an internucleotidic linkage is a moiety which does not a comprise a phosphorus but serves to link two natural or non-natural sugars.

In certain embodiments, a composition comprises a multimer of two or more of any: oligonucleotides of a first plurality and/or oligonucleotides of a second plurality, wherein the oligonucleotides of the first and second plurality can independently direct knockdown of the same or different targets independently via RNA interference and/or RNase H-mediated knockdown.

In certain embodiments, the present disclosure provides an oligonucleotide composition comprising a first plurality of oligonucleotides which share:
1) a common base sequence;
2) a common pattern of backbone linkages;
3) common stereochemistry independently at at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 chiral internucleotidic linkages (“chirally controlled internucleotidic linkages”); which composition is chirally controlled in that level of the first plurality of oligonucleotides in the composition is predetermined.

In certain embodiments, an oligonucleotide composition comprising a plurality of oligonucleotides (e.g., a first plurality of oligonucleotides) is chirally controlled in that oligonucleotides of the plurality share a common stereochemistry independently at one or more chiral internucleotidic linkages. In certain embodiments, oligonucleotides of the plurality share a common stereochemistry configuration at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 or more chiral internucleotidic linkages, each of which is independently Rp or Sp In certain embodiments, oligonucleotides of the plurality share a common stereochemistry configuration at each chiral internucleotidic linkages. In certain embodiments, a chiral internucleotidic linkage where a predetermined level of oligonucleotides of a composition share a common stereochemistry configuration (independently Rp or Sp) is referred to as a chirally controlled internucleotidic linkage.

In certain embodiments, a predetermined level of oligonucleotides of a provided composition, e.g., a first plurality of oligonucleotides of certain example compositions, comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 or more chirally controlled internucleotidic linkages.

In certain embodiments, at least 5 internucleotidic linkages are chirally controlled; in certain embodiments, at least 10 internucleotidic linkages are chirally controlled; in certain embodiments, at least 15 internucleotidic linkages are chirally controlled; in certain embodiments, each chiral internucleotidic linkage is chirally controlled.

In certain embodiments, 1%-100% of chiral internucleotidic linkages are chirally controlled. In certain embodiments, at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of chiral internucleotidic linkages are chirally controlled.

In certain embodiments, the present disclosure provides an oligonucleotide composition comprising a first plurality of oligonucleotides which share:
1) a common base sequence;
2) a common pattern of backbone linkages; and
3) a common pattern of backbone chiral centers, which composition is a substantially pure preparation of oligonucleotide in that a predetermined level of the oligonucleotides in the composition have the common base sequence and length, the common pattern of backbone linkages, and the common pattern of backbone chiral centers. In certain embodiments, the common pattern of backbone chiral centers comprises at least one internucleotidic linkage comprising a chirally controlled chiral center. In certain embodiments, a predetermined level of oligonucleotides is at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in a provided composition. In certain embodiments, a predetermined level of oligonucleotides is at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in a provided composition that are of or comprise a common base sequence. In certain embodiments, all oligonucleotides in a provided composition that are of or comprise a common base sequence are at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in the composition. In certain embodiments, a predetermined level of oligonucleotides is at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in a provided composition that are of or comprise a common base sequence, base modification, sugar modification and/or modified internucleotidic linkage. In certain embodiments, all oligonucleotides in a provided composition that are of or comprise a common base sequence, base modification, sugar modification and/or modified internucleotidic linkage are at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in the composition. In certain embodiments, a predetermined level of oligonucleotides is at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in a provided composition that are of or comprise a common base sequence, pattern of base modification, pattern of sugar modification, and/or pattern of modified internucleotidic linkage. In certain embodiments, all oligonucleotides in a provided composition that are of or comprise a common base sequence, pattern of base modification, pattern of sugar modification, and/or pattern of modified internucleotidic linkage are at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in the composition. In certain embodiments, a predetermined level of oligonucleotides is at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in a provided composition that share a common base sequence, a common pattern of base modification, a common pattern of sugar modification, and/or a common pattern of modified internucleotidic linkages. In certain embodiments, all oligonucleotides in a provided composition that share a common base sequence, a common pattern of base modification, a common pattern of sugar modification, and/or a common pattern of modified internucleotidic linkages are at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in the composition. In certain embodiments, a predetermined level is 1-100%. In certain embodiments, a predetermined level is at least 1%. In certain embodiments, a predetermined level is at least 5%. In certain embodiments, a predetermined level is at least 10%. In certain embodiments, a predetermined level is at least 20%. In certain embodiments, a predetermined level is at least 30%. In certain embodiments, a predetermined level is at least 40%. In certain embodiments, a predetermined level is at least 50%. In certain embodiments, a predetermined level is at least 60%. In certain embodiments, a predetermined level is at least 10%. In certain embodiments, a predetermined level is at least 70%. In certain embodiments, a predetermined level is at least 80%. In certain embodiments, a predetermined level is at least 90%. In certain embodiments, a predetermined level is at least 5*(1/2 g), wherein g is the number of chirally controlled internucleotidic linkages. In certain embodiments, a predetermined level is at least 10*(1/2 g), wherein g is the number of chirally controlled internucleotidic linkages. In certain embodiments, a predetermined level is at least 100*(1/2 g), wherein g is the number of chirally controlled internucleotidic linkages. In certain embodiments, a predetermined level is at least (0.80)g, wherein g is the number of chirally controlled internucleotidic linkages. In certain embodiments, a predetermined level is at least (0.80)g, wherein g is the number of chirally controlled internucleotidic linkages. In certain embodiments, a predetermined level is at least (0.80)g, wherein g is the number of chirally controlled internucleotidic linkages. In certain embodiments, a predetermined level is at least (0.85)g, wherein g is the number of chirally controlled internucleotidic linkages. In certain embodiments, a predetermined level is at least (0.90)g, wherein g is the number of chirally controlled internucleotidic linkages. In certain embodiments, a predetermined level is at least (0.95)g, wherein g is the number of chirally controlled internucleotidic linkages. In certain embodiments, a predetermined level is at least (0.96)g, wherein g is the number of chirally controlled internucleotidic linkages. In certain embodiments, a predetermined level is at least (0.97)g, wherein g is the number of chirally controlled internucleotidic linkages. In certain embodiments, a predetermined level is at least (0.98)g, wherein g is the number of chirally controlled internucleotidic linkages. In certain embodiments, a predetermined level is at least (0.99)g, wherein g is the number of chirally controlled internucleotidic linkages. In certain embodiments, to determine level of oligonucleotides having g chirally controlled internucleotidic linkages in a composition, product of diastereopurity of each of the g chirally controlled internucleotidic linkages: (diastereopurity of chirally controlled internucleotidic linkage 1)*(diastereopurity of chirally controlled internucleotidic linkage 2)* . . . *(diastereopurity of chirally controlled internucleotidic linkage g) is utilized as the level, wherein diastereopurity of each chirally controlled internucleotidic linkage is independently represented by diastereopurity of a dimer comprising the same internucleotidic linkage and nucleosides flanking the internucleotidic linkage and prepared under comparable methods as the oligonucleotides (e.g., comparable or preferably identical oligonucleotide preparation cycles, including comparable or preferably identical reagents and reaction conditions). In certain embodiments, levels of oligonucleotides and/or diastereopurity can be determined by analytical methods, e.g., chromatographic, spectrometric, spectroscopic methods or any combinations thereof. Among other things, the present disclosure encompasses the recognition that stereorandom oligonucleotide preparations contain a plurality of distinct chemical entities that differ from one another, e.g., in the stereochemical structure (or stereochemistry) of individual backbone chiral centers within the oligonucleotide chain. Without control of stereochemistry of backbone chiral centers, stereorandom oligonucleotide preparations provide uncontrolled compositions comprising undetermined levels of oligonucleotide stereoisomers. Even though these stereoisomers may have the same base sequence and/or chemical modifications, they are different chemical entities at least due to their different backbone stereochemistry, and they can have, as demonstrated herein, different properties, e.g., sensitivity to nucleases, activities, distribution, etc. In certain embodiments, a particular stereoisomer may be defined, for example, by its base sequence, its length, its pattern of backbone linkages, and its pattern of backbone chiral centers. In certain embodiments, the present disclosure demonstrates that improvements in properties and activities achieved through control of stereochemistry within an oligonucleotide can be comparable to, or even better than those achieved through use of chemical modification.

Among other things, the present disclosure encompasses the recognition that stereorandom oligonucleotide preparations contain a plurality of distinct chemical entities that differ from one another, e.g., in the stereochemical structure (or stereochemistry) of individual backbone chiral centers within the oligonucleotide chain. Without control of stereochemistry of backbone chiral centers, stereorandom oligonucleotide preparations provide uncontrolled compositions comprising undetermined levels of oligonucleotide stereoisomers. Even though these stereoisomers may have the same base sequence and/or chemical modifications, they are different chemical entities at least due to their different backbone stereochemistry, and they can have, as demonstrated herein, different properties, e.g., sensitivity to nucleases, activities, distribution, etc. In certain embodiments, a particular stereoisomer may be defined, for example, by its base sequence, its length, its pattern of backbone linkages, and its pattern of backbone chiral centers. In certain embodiments, the present disclosure demonstrates that improvements in properties and activities achieved through control of stereochemistry within an oligonucleotide can be comparable to, or even better than those achieved through use of chemical modification

In some embodiments, a ds oligonucleotide targeting HSD17B13 or ds oligonucleotide targeting HSD17B13 composition is useful for prevention or treatment of a HSD17B13-associated condition, disorder, or disease, in a subject in need thereof. In some embodiments, the present disclosure provides a method for preventing or treating a HSD17B13-associated condition, disorder, or disease, comprising administering to a subject suffering therefrom or subject thereto a therapeutically effective amount of a provided ds oligonucleotide or a pharmaceutical composition that can deliver or comprise a therapeutically effective amount of a provided ds oligonucleotide. In some embodiments, the present disclosure provides pharmaceutical compositions which comprise a provided ds oligonucleotide targeting HSD17B13 and a pharmaceutically acceptable carrier. In some embodiments, oligonucleotides in a pharmaceutical composition are in one or more pharmaceutically acceptable salt forms, e.g., a sodium salt form, an ammonium salt form, etc.

In some embodiments, an oligonucleotide or oligonucleotide composition is useful for the manufacture of a medicament for prevention or treatment of a HSD17B13-associated condition, disorder, or disease, such as NAFLD, NASH, ASH, alcoholic liver disease, nonalcoholic liver disease, alcoholic cirrhosis, nonalcoholic cirrhosis, steatohepatitis, hepatic steatosis, hepatoceullar carcinoma, HCV hepatitis, chronic hepatitis, hereditary hemochromatosis, primary sclerosing cholangitis, drug induced liver injury, or hepatocellular necrosis, in a subject in need thereof.

Various HSD17B13-associated conditions, disorders, or diseases may be prevented and/or treated utilizing provided technologies (e.g., oligonucleotide, compositions, methods, etc.). In some embodiments, a condition, disorder, or disease is NAFLD. In some embodiments, a condition, disorder, or disease is NASH. In some embodiments, a condition, disorder, or disease is ASH.

### DETAILED DESCRIPTION OF CERTAIN EMBODIMENTS

Technologies of the present disclosure may be understood more readily by reference to the following detailed description of certain embodiments.

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included.

Unless otherwise specified, description of oligonucleotides and elements thereof (e.g., base sequence, sugar modifications, internucleotidic linkages, linkage phosphorus stereochemistry, patterns thereof, etc.) is from 5′ to 3′. As those skilled in the art will appreciate, in some embodiments, oligonucleotides may be provided and/or utilized as salt forms, particularly pharmaceutically acceptable salt forms, e.g., sodium salts. Unless otherwise indicated, oligonucleotides include various forms of the oligonucleotides. As those skilled in the art will also appreciate, in some embodiments, individual oligonucleotides within a composition may be considered to be of the same constitution and/or structure even though, within such composition (e.g., a liquid composition), particular such oligonucleotides might be in different salt form(s) (and may be dissolved and the oligonucleotide chain may exist as an anion form when, e.g., in a liquid composition) at a particular moment in time. For example, those skilled in the art will appreciate that, at a given pH, individual internucleotidic linkages along an oligonucleotide chain may be in an acid (H) form, or in one of a plurality of possible salt forms (e.g., a sodium salt, or a salt of a different cation, depending on which ions might be present in the preparation or composition), and will understand that, so long as their acid forms (e.g., replacing all cations, if any, with H⁺) are of the same constitution and/or structure, such individual oligonucleotides may properly be considered to be of the same constitution and/or structure.

Analog: The term “analog” includes any chemical moiety which differs structurally from a reference chemical moiety or class of moieties, but which is capable of performing at least one function of such a reference chemical moiety or class of moieties. As non-limiting examples, a nucleotide analog differs structurally from a nucleotide but performs at least one function of a nucleotide; a nucleobase analog differs structurally from a nucleobase but performs at least one function of a nucleobase; etc.

Antisense: The term “antisense”, as used herein, refers to a characteristic of an oligonucleotide or other nucleic acid having a base sequence complementary or substantially complementary to a target nucleic acid to which it is capable of hybridizing. In some embodiments, a target nucleic acid is a target gene mRNA. In some embodiments, hybridization is required for or results in at one activity, e.g., a decrease in the level, expression or activity of the target nucleic acid or a gene product thereof. The term “antisense oligonucleotide”, as used herein, refers to an oligonucleotide complementary to a target nucleic acid. In some embodiments, an antisense oligonucleotide is capable of directing a decrease in the level, expression or activity of a target nucleic acid or a product thereof. In some embodiments, an antisense oligonucleotide is capable of directing a decrease in the level, expression or activity of the target nucleic acid or a product thereof, via a mechanism that involves RNA interference.

Chiral control: As used herein, “chiral control” refers to control of the stereochemical designation of the chiral linkage phosphorus in a chiral internucleotidic linkage within an oligonucleotide. As used herein, a chiral internucleotidic linkage is an internucleotidic linkage whose linkage phosphorus is chiral. In some embodiments, a control is achieved through a chiral element that is absent from the sugar and base moieties of an oligonucleotide, for example, in some embodiments, a control is achieved through use of one or more chiral auxiliaries during oligonucleotide preparation as described in the present disclosure, which chiral auxiliaries often are part of chiral phosphoramidites used during oligonucleotide preparation. In contrast to chiral control, a person having ordinary skill in the art appreciates that conventional oligonucleotide synthesis which does not use chiral auxiliaries cannot control stereochemistry at a chiral internucleotidic linkage if such conventional oligonucleotide synthesis is used to form the chiral internucleotidic linkage. In some embodiments, the stereochemical designation of each chiral linkage phosphorus in each chiral internucleotidic linkage within an oligonucleotide is controlled.

Chirally controlled oligonucleotide composition: The terms “chirally controlled oligonucleotide composition”, “chirally controlled nucleic acid composition”, and the like, as used herein, refers to a composition that comprises a plurality of oligonucleotides (or nucleic acids) which share 1) a common base sequence, 2) a common pattern of backbone linkages, and 3) a common pattern of backbone phosphorus modifications, wherein the plurality of oligonucleotides (or nucleic acids) share the same linkage phosphorus stereochemistry at one or more chiral internucleotidic linkages (chirally controlled or stereodefined internucleotidic linkages, whose chiral linkage phosphorus is Rp or Sp in the composition (“stereodefined”), not a random Rp and Sp mixture as non-chirally controlled internucleotidic linkages). Level of the plurality of oligonucleotides (or nucleic acids) in a chirally controlled oligonucleotide composition is pre-determined/controlled (e.g., through chirally controlled oligonucleotide preparation to stereoselectively form one or more chiral internucleotidic linkages). In some embodiments, about 1%-100%, (e.g., about 5%/o-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition are oligonucleotides of the plurality. In some embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition that share the common base sequence, the common pattern of backbone linkages, and the common pattern of backbone phosphorus modifications are oligonucleotides of the plurality. In some embodiments, a level is about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a composition, or of all oligonucleotides in a composition that share a common base sequence (e.g., of a plurality of oligonucleotide or an oligonucleotide type), or of all oligonucleotides in a composition that share a common base sequence, a common pattern of backbone linkages, and a common pattern of backbone phosphorus modifications, or of all oligonucleotides in a composition that share a common base sequence, a common patter of base modifications, a common pattern of sugar modifications, a common pattern of internucleotidic linkage types, and/or a common pattern of internucleotidic linkage modifications. In some embodiments, the plurality of oligonucleotides share the same stereochemistry at about 1-50 chiral internucleotidic linkages. In some embodiments, the plurality of oligonucleotides share the same stereochemistry at about 1%-100% of chiral internucleotidic linkages. In some embodiments, oligonucleotides (or nucleic acids) of a plurality are of the same constitution (as appreciated by those skilled in the art, in some embodiments may exist in one or more forms, e.g., acid forms, salt forms, etc.). In some embodiments, level of the oligonucleotides (or nucleic acids) of the plurality is about 1%-100% of all oligonucleotides (or nucleic acids) in a composition that share the same constitution as the oligonucleotides (or nucleic acids) of the plurality. In some embodiments, each chiral internucleotidic linkage is a chiral controlled internucleotidic linkage, and the composition is a completely chirally controlled oligonucleotide composition. In some embodiments, oligonucleotides (or nucleic acids) of a plurality are structurally identical. In some embodiments, a chirally controlled internucleotidic linkage has a diastereopurity of at least 80%, 85%, 90%, 91%, 92%, 93%, 94%, 95%, 96%, 97%, 98%, 99% or 99.5%, typically at least 90%, 91%, 92%, 93%, 94%, 95%, 96%, 97%, 98%, 99% or 99.5%. In some embodiments, a chirally controlled internucleotidic linkage has a diastereopurity of at least 95%. In some embodiments, a chirally controlled internucleotidic linkage has a diastereopurity of at least 96%. In some embodiments, a chirally controlled internucleotidic linkage has a diastereopurity of at least 97%. In some embodiments, a chirally controlled internucleotidic linkage has a diastereopurity of at least 98%. In some embodiments, a chirally controlled internucleotidic linkage has a diastereopurity of at least 99%. In some embodiments, a percentage (e.g., a level as described herein) is or is at least (DS)^{nc}, wherein DS is a diastereopurity as described in the present disclosure (e.g., 90%, 91%, 92%, 93%, 94%, 95%, 96%, 97%, 98%, 99% or 99.5% or more) and nc is the number of chirally controlled internucleotidic linkages as described in the present disclosure (e.g., 1-50, 1-40, 1-30, 1-25, 1-20, 5-50, 5-40, 5-30, 5-25, 5-20, 1, 2, 3, 4, 5, 6, 7, 8, 9, 10, 11, 12, 13, 14, 15, 16, 17, 18, 19, 20, 21, 22, 23, 24, 25 or more). In some embodiments, a percentage (e.g., a level as described herein) is or is at least (DS)^{nc}, wherein DS is 95%-100%. For example, when DS is 99% and nc is 10, the percentage is or is at least 90% ((99%)¹⁰≈0.90=90%). In some embodiments, level of a plurality of oligonucleotides in a composition is represented as the product of the diastereopurity of each chirally controlled internucleotidic linkage in the oligonucleotides. In some embodiments, diastereopurity of an internucleotidic linkage connecting two nucleosides in an oligonucleotide (or nucleic acid) is represented by the diastereopurity of an internucleotidic linkage of a dimer connecting the same two nucleosides, wherein the dimer is prepared using comparable conditions, in some instances, identical synthetic cycle conditions (e.g., for the linkage between Nx and Ny in an oligonucleotide . . . NxNy . . . , the dimer is NxNy). In some embodiments, not all chiral internucleotidic linkages are chiral controlled internucleotidic linkages, and the composition is a partially chirally controlled oligonucleotide composition. In some embodiments, a non-chirally controlled internucleotidic linkage has a diastereopurity of less than about 80%, 75%, 70%, 65%, 60%, 55%, or of about 50%, as typically observed in stereorandom oligonucleotide compositions (e.g., as appreciated by those skilled in the art, from traditional oligonucleotide synthesis, e.g., the phosphoramidite method). In some embodiments, oligonucleotides (or nucleic acids) of a plurality are of the same type. In some embodiments, a chirally controlled oligonucleotide composition comprises non-random or controlled levels of individual oligonucleotide or nucleic acids types. For instance, in some embodiments a chirally controlled oligonucleotide composition comprises one and no more than one oligonucleotide type. In some embodiments, a chirally controlled oligonucleotide composition comprises more than one oligonucleotide type. In some embodiments, a chirally controlled oligonucleotide composition comprises multiple oligonucleotide types. In some embodiments, a chirally controlled oligonucleotide composition is a composition of oligonucleotides of an oligonucleotide type, which composition comprises a non-random or controlled level of a plurality of oligonucleotides of the oligonucleotide type.

Internucleotidic linkage: As used herein, the phrase “internucleotidic linkage” refers generally to a linkage linking nucleoside units of an oligonucleotide or a nucleic acid. In some embodiments, an internucleotidic linkage is a phosphodiester linkage, as extensively found in naturally occurring DNA and RNA molecules (natural phosphate linkage (—OP(═O)(OH)O—), which as appreciated by those skilled in the art may exist as a salt form). In some embodiments, an internucleotidic linkage is a modified internucleotidic linkage (not a natural phosphate linkage). In some embodiments, an internucleotidic linkage is a “modified internucleotidic linkage” wherein at least one oxygen atom or —OH of a phosphodiester linkage is replaced by a different organic or inorganic moiety. In some embodiments, such an organic or inorganic moiety is selected from ═S, ═Se, ═NR′, —SR′, —SeR′, —N(R′)₂, B(R′)₃, —S—, —Se—, and —N(R′)—, wherein each R′ is independently as defined and described in the present disclosure. In some embodiments, an internucleotidic linkage is a phosphotriester linkage, phosphorothioate linkage (or phosphorothioate diester linkage, —OP(═O)(SH)O—, which as appreciated by those skilled in the art may exist as a salt form), or phosphorothioate triester linkage. In some embodiments, a modified internucleotidic linkage is a phosphorothioate linkage. In some embodiments, an internucleotidic linkage is one of, e.g., PNA (peptide nucleic acid) or PMO (phosphorodiamidate Morpholino oligomer) linkage. In some embodiments, a modified internucleotidic linkage is a non-negatively charged internucleotidic linkage. In some embodiments, a modified internucleotidic linkage is a neutral internucleotidic linkage (e.g., n001 in certain provided oligonucleotides). It is understood by a person of ordinary skill in the art that an internucleotidic linkage may exist as an anion or cation at a given pH due to the existence of acid or base moieties in the linkage. In some embodiments, a modified internucleotidic linkages is a modified internucleotidic linkages designated as s, s1, s2, s3, s4, s5, s6, s7, s8, s9, s10, s11, s12, s13, s14, s15, s16, s17 and s18 as described in WO 2017/210647.

In vitro: As used herein, the term “in vitro” refers to events that occur in an artificial environment, e.g., in a test tube or reaction vessel, in cell culture, etc., rather than within an organism (e.g., animal, plant and/or microbe).

In vivo: As used herein, the term “in vivo” refers to events that occur within an organism (e.g., animal, plant and/or microbe).

Linkage phosphorus: as defined herein, the phrase “linkage phosphorus” is used to indicate that the particular phosphorus atom being referred to is the phosphorus atom present in the internucleotidic linkage, which phosphorus atom corresponds to the phosphorus atom of a phosphodiester internucleotidic linkage as occurs in naturally occurring DNA and RNA. In some embodiments, a linkage phosphorus atom is in a modified internucleotidic linkage, wherein each oxygen atom of a phosphodiester linkage is optionally and independently replaced by an organic or inorganic moiety. In some embodiments, a linkage phosphorus atom is the P of Formula I as described herein. In some embodiments, a linkage phosphorus atom is chiral. In some embodiments, a linkage phosphorus atom is achiral (e.g., as in natural phosphate linkages).

Linker: The terms “linker”, “linking moiety” and the like refer to any chemical moiety which connects one chemical moiety to another. As appreciated by those skilled in the art, a linker can be bivalent or trivalent or more, depending on the number of chemical moieties the linker connects. In some embodiments, a linker is a moiety which connects one oligonucleotide to another oligonucleotide in a multimer. In some embodiments, a linker is a moiety optionally positioned between the terminal nucleoside and the solid support or between the terminal nucleoside and another nucleoside, nucleotide, or nucleic acid. In some embodiments, in an oligonucleotide a linker connects a chemical moiety (e.g., a targeting moiety, a lipid moiety, a carbohydrate moiety, etc.) with an oligonucleotide chain (e.g., through its 5′-end, 3′-end, nucleobase, sugar, internucleotidic linkage, etc.)

Modified nucleobase: The terms “modified nucleobase”, “modified base” and the like refer to a chemical moiety which is chemically distinct from a nucleobase, but which is capable of performing at least one function of a nucleobase. In some embodiments, a modified nucleobase is a nucleobase which comprises a modification. In some embodiments, a modified nucleobase is capable of at least one function of a nucleobase, e.g., forming a moiety in a polymer capable of base-pairing to a nucleic acid comprising an at least complementary sequence of bases. In some embodiments, a modified nucleobase is substituted A, T, C, G, or U, or a substituted tautomer of A, T, C, G, or U. In some embodiments, a modified nucleobase in the context of oligonucleotides refer to a nucleobase that is not A, T, C, G or U.

Modified nucleoside: The term “modified nucleoside” refers to a moiety derived from or chemically similar to a natural nucleoside, but which comprises a chemical modification which differentiates it from a natural nucleoside. Non-limiting examples of modified nucleosides include those which comprise a modification at the base and/or the sugar. Non-limiting examples of modified nucleosides include those with a 2′ modification at a sugar. Non-limiting examples of modified nucleosides also include abasic nucleosides (which lack a nucleobase). In some embodiments, a modified nucleoside is capable of at least one function of a nucleoside, e.g., forming a moiety in a polymer capable of base-pairing to a nucleic acid comprising an at least complementary sequence of bases.

Modified nucleotide: The term “modified nucleotide” includes any chemical moiety which differs structurally from a natural nucleotide but is capable of performing at least one function of a natural nucleotide. In some embodiments, a modified nucleotide comprises a modification at a sugar, base and/or internucleotidic linkage. In some embodiments, a modified nucleotide comprises a modified sugar, modified nucleobase and/or modified internucleotidic linkage. In some embodiments, a modified nucleotide is capable of at least one function of a nucleotide, e.g., forming a subunit in a polymer capable of base-pairing to a nucleic acid comprising an at least complementary sequence of bases.

Modified sugar: The term “modified sugar” refers to a moiety that can replace a sugar. A modified sugar mimics the spatial arrangement, electronic properties, or some other physicochemical property of a sugar. In some embodiments, as described in the present disclosure, a modified sugar is substituted ribose or deoxyribose. In some embodiments, a modified sugar comprises a 2′-modification. Examples of useful 2′-modification are widely utilized in the art and described herein. In some embodiments, a 2′-modification is 2′-OR, wherein R is optionally substituted C₁₋₁₀ aliphatic. In some embodiments, a 2′-modification is 2′-OMe. In some embodiments, a 2′-modification is 2′-MOE. In some embodiments, a modified sugar is a bicyclic sugar (e.g., a sugar used in LNA, BNA, etc.). In some embodiments, in the context of oligonucleotides, a modified sugar is a sugar that is not ribose or deoxyribose as typically found in natural RNA or DNA.

Nucleic acid: The term “nucleic acid”, as used herein, includes any nucleotides and polymers thereof. The term “polynucleotide”, as used herein, refers to a polymeric form of nucleotides of any length, either ribonucleotides (RNA) or deoxyribonucleotides (DNA) or a combination thereof. These terms refer to the primary structure of the molecules and, thus, include double- and single-stranded DNA, and double- and single-stranded RNA. These terms include, as equivalents, analogs of either RNA or DNA comprising modified nucleotides and/or modified polynucleotides, such as, though not limited to, methylated, protected and/or capped nucleotides or polynucleotides. The terms encompass poly- or oligo-ribonucleotides (RNA) and poly- or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified internucleotidic linkages. The term encompasses nucleic acids containing any combinations of nucleobases, modified nucleobases, sugars, modified sugars, phosphate bridges or modified internucleotidic linkages. Examples include, and are not limited to, nucleic acids containing ribose moieties, nucleic acids containing deoxy-ribose moieties, nucleic acids containing both ribose and deoxyribose moieties, nucleic acids containing ribose and modified ribose moieties. Unless otherwise specified, the prefix poly- refers to a nucleic acid containing 2 to about 10,000 nucleotide monomer units and wherein the prefix oligo- refers to a nucleic acid containing 2 to about 200 nucleotide monomer units.

Nucleobase: The term “nucleobase” refers to the parts of nucleic acids that are involved in the hydrogen-bonding that binds one nucleic acid strand to another complementary strand in a sequence specific manner. The most common naturally-occurring nucleobases are adenine (A), guanine (G), uracil (U), cytosine (C), and thymine (T). In some embodiments, a naturally-occurring nucleobases are modified adenine, guanine, uracil, cytosine, or thymine. In some embodiments, a naturally-occurring nucleobases are methylated adenine, guanine, uracil, cytosine, or thymine. In some embodiments, a nucleobase comprises a heteroaryl ring wherein a ring atom is nitrogen, and when in a nucleoside, the nitrogen is bonded to a sugar moiety. In some embodiments, a nucleobase comprises a heterocyclic ring wherein a ring atom is nitrogen, and when in a nucleoside, the nitrogen is bonded to a sugar moiety. In some embodiments, a nucleobase is a “modified nucleobase,” a nucleobase other than adenine (A), guanine (G), uracil (U), cytosine (C), and thymine (T). In some embodiments, a modified nucleobase is substituted A, T, C, G or U. In some embodiments, a modified nucleobase is a substituted tautomer of A, T, C, G, or U. In some embodiments, a modified nucleobases is methylated adenine, guanine, uracil, cytosine, or thymine. In some embodiments, a modified nucleobase mimics the spatial arrangement, electronic properties, or some other physicochemical property of the nucleobase and retains the property of hydrogen-bonding that binds one nucleic acid strand to another in a sequence specific manner. In some embodiments, a modified nucleobase can pair with all of the five naturally occurring bases (uracil, thymine, adenine, cytosine, or guanine) without substantially affecting the melting behavior, recognition by intracellular enzymes or activity of the oligonucleotide duplex. As used herein, the term “nucleobase” also encompasses structural analogs used in lieu of natural or naturally-occurring nucleotides, such as modified nucleobases and nucleobase analogs. In some embodiments, a nucleobase is optionally substituted A, T, C, G, or U, or an optionally substituted tautomer of A, T, C, G, or U. In some embodiments, a “nucleobase” refers to a nucleobase unit in an oligonucleotide or a nucleic acid (e.g., A, T, C, G or U as in an oligonucleotide or a nucleic acid).

Nucleoside: The term “nucleoside” refers to a moiety wherein a nucleobase or a modified nucleobase is covalently bound to a sugar or a modified sugar. In some embodiments, a nucleoside is a natural nucleoside, e.g., adenosine, deoxyadenosine, guanosine, deoxyguanosine, thymidine, uridine, cytidine, or deoxycytidine. In some embodiments, a nucleoside is a modified nucleoside, e.g., a substituted natural nucleoside selected from adenosine, deoxyadenosine, guanosine, deoxyguanosine, thymidine, uridine, cytidine, and deoxycytidine. In some embodiments, a nucleoside is a modified nucleoside, e.g., a substituted tautomer of a natural nucleoside selected from adenosine, deoxyadenosine, guanosine, deoxyguanosine, thymidine, uridine, cytidine, and deoxycytidine. In some embodiments, a “nucleoside” refers to a nucleoside unit in an oligonucleotide or a nucleic acid.

Nucleotide: The term “nucleotide” as used herein refers to a monomeric unit of a polynucleotide that consists of a nucleobase, a sugar, and one or more internucleotidic linkages (e.g., phosphate linkages in natural DNA and RNA). The naturally occurring bases [guanine, (G), adenine, (A), cytosine, (C), thymine, (T), and uracil (U)] are derivatives of purine or pyrimidine, though it should be understood that naturally and non-naturally occurring base analogs are also included. The naturally occurring sugar is the pentose (five-carbon sugar) deoxyribose (which forms DNA) or ribose (which forms RNA), though it should be understood that naturally and non-naturally occurring sugar analogs are also included. Nucleotides are linked via internucleotidic linkages to form nucleic acids, or polynucleotides. Many internucleotidic linkages are known in the art (such as, though not limited to, phosphate, phosphorothioates, boranophosphates and the like). Artificial nucleic acids include PNAs (peptide nucleic acids), phosphotriesters, phosphorothionates, H-phosphonates, phosphoramidates, boranophosphates, methylphosphonates, phosphonoacetates, thiophosphonoacetates and other variants of the phosphate backbone of native nucleic acids, such as those described herein. In some embodiments, a natural nucleotide comprises a naturally occurring base, sugar and internucleotidic linkage. As used herein, the term “nucleotide” also encompasses structural analogs used in lieu of natural or naturally-occurring nucleotides, such as modified nucleotides and nucleotide analogs. In some embodiments, a “nucleotide” refers to a nucleotide unit in an oligonucleotide or a nucleic acid.

Oligonucleotide: The term “oligonucleotide” refers to a polymer or oligomer of nucleotides, and may contain any combination of natural and non-natural nucleobases, sugars, and internucleotidic linkages.

Oligonucleotides can be single-stranded or double-stranded. A single-stranded oligonucleotide can have double-stranded regions (formed by two portions of the single-stranded oligonucleotide) and a double-stranded oligonucleotide, which comprises two oligonucleotide chains, can have single-stranded regions for example, at regions where the two oligonucleotide chains are not complementary to each other. Example oligonucleotides include, but are not limited to structural genes, genes including control and termination regions, self-replicating systems such as viral or plasmid DNA, single-stranded and double-stranded RNAi agents and other RNA interference reagents (RNAi agents or iRNA agents), shRNA, antisense oligonucleotides, ribozymes, microRNAs, microRNA mimics, supermirs, aptamers, antimirs, antagomirs, Ul adaptors, triplex-forming oligonucleotides, G-quadruplex oligonucleotides, RNA activators, immuno-stimulatory oligonucleotides, and decoy oligonucleotides.

Oligonucleotides of the present disclosure can be of various lengths. In particular embodiments, oligonucleotides can range from about 2 to about 200 nucleosides in length. In various related embodiments, oligonucleotides, single-stranded, double-stranded, or triple-stranded, can range in length from about 4 to about 10 nucleosides, from about 10 to about 50 nucleosides, from about 20 to about 50 nucleosides, from about 15 to about 30 nucleosides, from about 20 to about 30 nucleosides in length. In some embodiments, the oligonucleotide is from about 9 to about 39 nucleosides in length. In some embodiments, the oligonucleotide is at least 4, 5, 6, 7, 8, 9, 10, 11, 12, 13, 14, 15, 16, 17, 18, 19, 20, 21, 22, 23, 24, or 25 nucleosides in length. In some embodiments, the oligonucleotide is at least 19 nucleosides in length. In some embodiments, the oligonucleotide is at least 20 nucleosides in length. In some embodiments, the oligonucleotide is at least 25 nucleosides in length. In some embodiments, the oligonucleotide is at least 30 nucleosides in length. In some embodiments, the oligonucleotide is a duplex of complementary strands of at least 18 nucleosides in length. In some embodiments, the oligonucleotide is a duplex of complementary strands of at least 21 nucleosides in length. In some embodiments, each nucleoside counted in an oligonucleotide length independently comprises A, T, C, G, or U, or optionally substituted A, T, C, G, or U, or an optionally substituted tautomer of A, T, C, G or U.

Oligonucleotide type: As used herein, the phrase “oligonucleotide type” is used to define an oligonucleotide that has a particular base sequence, pattern of backbone linkages (i.e., pattern of internucleotidic linkage types, for example, phosphate, phosphorothioate, phosphorothioate triester, etc.), pattern of backbone chiral centers [i.e., pattern of linkage phosphorus stereochemistry (Rp/Sp)], and pattern of backbone phosphorus modifications (e.g., pattern of “—XLR¹” groups in Formula I as described herein). In some embodiments, oligonucleotides of a common designated “type” are structurally identical to one another.

One of skill in the art will appreciate that synthetic methods of the present disclosure provide for a degree of control during the synthesis of an oligonucleotide strand such that each nucleotide unit of the oligonucleotide strand can be designed and/or selected in advance to have a particular stereochemistry at the linkage phosphorus and/or a particular modification at the linkage phosphorus, and/or a particular base, and/or a particular sugar. In some embodiments, an oligonucleotide strand is designed and/or selected in advance to have a particular combination of stereocenters at the linkage phosphorus. In some embodiments, an oligonucleotide strand is designed and/or determined to have a particular combination of modifications at the linkage phosphorus. In some embodiments, an oligonucleotide strand is designed and/or selected to have a particular combination of bases. In some embodiments, an oligonucleotide strand is designed and/or selected to have a particular combination of one or more of the above structural characteristics. In some embodiments, the present disclosure provides compositions comprising or consisting of a plurality of oligonucleotide molecules (e.g., chirally controlled oligonucleotide compositions). In some embodiments, all such molecules are of the same type (i.e., are structurally identical to one another). In some embodiments, however, provided compositions comprise a plurality of oligonucleotides of different types, typically in pre-determined relative amounts.

Optionally Substituted: As described herein, compounds, e.g., oligonucleotides, of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. In some embodiments, an optionally substituted group is unsubstituted. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. Certain substituents are described below.

Suitable monovalent substituents on a substitutable atom, e.g., a suitable carbon atom, are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —Si(R^{∘})₃; —OSi(R^{∘})₃; —B(R^{∘})₂; —OB(R^{∘})₂; —OB(OR^{∘})₂; —P(R^{∘})₂; —P(OR^{∘})₂; —P(R^{∘})(OR^{∘}); —OP(R^{∘})₂; —OP(OR^{∘})₂; —OP(R^{∘})(OR^{∘}); —P(O)(R^{∘})₂; —P(O)(OR^{∘})₂; —OP(O)(R^{∘})₂; —OP(O)(OR^{∘})₂; —OP(O)(OR^{∘})(SR^{∘}); —SP(O)(R^{∘})₂; —SP(O)(OR^{∘})₂; —N(R^{∘})P(O)(R^{∘})₂; —N(R^{∘})P(O)(OR^{∘})₂; —P(R^{∘})₂[B(R^{∘})₃]; —P(OR^{∘})₂[B(R^{∘})₃]; —OP(R^{∘})₂[B(R^{∘})₃]; —OP(OR^{∘})₂[B(R^{∘})₃]; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined herein and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₁₋₄ aryl), —O(CH₂)₀₋₁(C₆₋₁₄ aryl), —CH₂-(5-14 membered heteroaryl ring), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{∘}, taken together with their intervening atom(s), form a 5-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR′2, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, and a 5-6-membered saturated, partially unsaturated, or aryl ring having 0-4 heteroatoms independently selected from nitrogen, oxygen, and sulfur. Suitable divalent substituents on a saturated carbon atom of R^{∘} include ═O and ═S.

Suitable divalent substituents, e.g., on a suitable carbon atom, are independently the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5-6-membered saturated, partially unsaturated, and aryl ring having 0-4 heteroatoms independently selected from nitrogen, oxygen, and sulfur.

Suitable substituents on the aliphatic group of R* are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

In some embodiments, suitable substituents on a substitutable nitrogen are independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12-membered saturated, partially unsaturated, or aryl mono- or bicyclic ring having 04 heteroatoms independently selected from nitrogen, oxygen, and sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Oral: The phrases “oral administration” and “administered orally” as used herein have their art-understood meaning referring to administration by mouth of a compound or composition.

Parenteral: The phrases “parenteral administration” and “administered parenterally” as used herein have their art-understood meaning referring to modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Pharmaceutical composition: As used herein, the term “pharmaceutical composition” refers to an active agent, formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, a provided compound comprises one or more acidic groups, e.g., an oligonucleotide, and a pharmaceutically acceptable salt is an alkali, alkaline earth metal, or ammonium (e.g., an ammonium salt of N(R)₃, wherein each R is independently defined and described in the present disclosure) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises more than one acid groups, for example, an oligonucleotide may comprise two or more acidic groups (e.g., in natural phosphate linkages and/or modified internucleotidic linkages). In some embodiments, a pharmaceutically acceptable salt, or generally a salt, of such a compound comprises two or more cations, which can be the same or different. In some embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in some embodiments, no more than about 7; in some embodiments, no more than about 6; in some embodiments, no more than about 5; in some embodiments, no more than about 4; in some embodiments, no more than about 3) in the acidic groups are replaced with cations. In some embodiments, each phosphorothioate and phosphate group independently exists in its salt form (e.g., if sodium salt, —O—P(O)(SNa)—O— and —O—P(O)(ONa)—O—, respectively). In some embodiments, each phosphorothioate and phosphate internucleotidic linkage independently exists in its salt form (e.g., if sodium salt, —O—P(O)(SNa)—O— and —O—P(O)(ONa)—O—, respectively). In some embodiments, a pharmaceutically acceptable salt is a sodium salt of an oligonucleotide. In some embodiments, a pharmaceutically acceptable salt is a sodium salt of an oligonucleotide, wherein each acidic phosphate and modified phosphate group (e.g., phosphorothioate, phosphate, etc.), if any, exists as a salt form (all sodium salt).

Protecting group: The term “protecting group,” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. 06/2012, the entirety of Chapter 2 is incorporated herein by reference. Suitable amino-protecting groups include but are not limited to described herein and/or in: WO 2018/022473, WO 2018/098264, WO 2018/223056, WO 2018/223073, WO 2018/223081, WO 2018/237194, WO 2019/032607, WO 2019/055951, and/or WO 2019/075357, or U.S. Provisional patent applications 62/825,766 and 62/911,339, the description of the protecting groups of each of which is independently incorporated herein by reference.

Sample: As used herein, the term “sample” typically refers to an aliquot of material obtained or derived from a source of interest. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secreations, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brocheoalvealar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Subject: As used herein, the term “subject” or “test subject” refers to any organism to which a provided compound (e.g., a provided oligonucleotide) or composition is administered in accordance with the present disclosure e.g., for experimental, diagnostic, prophylactic and/or therapeutic purposes. Typical subjects include animals (e.g., mammals such as mice, rats, rabbits, non-human primates, and humans; insects; worms; etc.) and plants. In some embodiments, a subject is a human. In some embodiments, a subject may be suffering from and/or susceptible to a disease, disorder and/or condition.

Substantially: As used herein, the term “substantially” refers to the qualitative condition of exhibiting total or near-total extent or degree of a characteristic or property of interest. A base sequence which is substantially complementary to a second sequence is not identical to the second sequence, but is mostly or nearly identical to the second sequence. In addition, one of ordinary skill in the biological and/or chemical arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Sugar: The term “sugar” refers to a monosaccharide or polysaccharide in closed and/or open form. In some embodiments, sugars are monosaccharides. In some embodiments, sugars are polysaccharides. Sugars include, but are not limited to, ribose, deoxyribose, pentofuranose, pentopyranose, and hexopyranose moieties. As used herein, the term “sugar” also encompasses structural analogs used in lieu of conventional sugar molecules, such as glycol, polymer of which forms the backbone of the nucleic acid analog, glycol nucleic acid (“GNA”), etc. As used herein, the term “sugar” also encompasses structural analogs used in lieu of natural or naturally-occurring nucleotides, such as modified sugars and nucleotide sugars. In some embodiments, a sugar is a RNA or DNA sugar (ribose or deoxyribose). In some embodiments, a sugar is a modified ribose or deoxyribose sugar, e.g., 2′-modified, 5′-modified, etc. As described herein, in some embodiments, when used in oligonucleotides and/or nucleic acids, modified sugars may provide one or more desired properties, activities, etc. In some embodiments, a sugar is optionally substituted ribose or deoxyribose. In some embodiments, a “sugar” refers to a sugar unit in an oligonucleotide or a nucleic acid.

Susceptible to: An individual who is “susceptible to” a disease, disorder and/or condition is one who has a higher risk of developing the disease, disorder and/or condition than does a member of the general public. In some embodiments, an individual who is susceptible to a disease, disorder and/or condition is predisposed to have that disease, disorder and/or condition. In some embodiments, an individual who is susceptible to a disease, disorder and/or condition may not have been diagnosed with the disease, disorder and/or condition. In some embodiments, an individual who is susceptible to a disease, disorder and/or condition may exhibit symptoms of the disease, disorder and/or condition. In some embodiments, an individual who is susceptible to a disease, disorder and/or condition may not exhibit symptoms of the disease, disorder and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic agent: As used herein, the term “therapeutic agent” in general refers to any agent that elicits a desired effect (e.g., a desired biological, clinical, or pharmacological effect) when administered to a subject. In some embodiments, an agent is considered to be a therapeutic agent if it demonstrates a statistically significant effect across an appropriate population. In some embodiments, an appropriate population is a population of subjects suffering from and/or susceptible to a disease, disorder or condition. In some embodiments, an appropriate population is a population of model organisms. In some embodiments, an appropriate population may be defined by one or more criterion such as age group, gender, genetic background, preexisting clinical conditions, prior exposure to therapy. In some embodiments, a therapeutic agent is a substance that alleviates, ameliorates, relieves, inhibits, prevents, delays onset of, reduces severity of, and/or reduces incidence of one or more symptoms or features of a disease, disorder, and/or condition in a subject when administered to the subject in an effective amount. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, a therapeutic agent is a provided compound, e.g., a provided oligonucleotide.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unsaturated: The term “unsaturated,” as used herein, means that a moiety has one or more units of unsaturation.

Wild-type: As used herein, the term “wild-type” has its art-understood meaning that refers to an entity having a structure and/or activity as found in nature in a “normal” (as contrasted with mutant, diseased, altered, etc.) state or context. Those of ordinary skill in the art will appreciate that wild type genes and polypeptides often exist in multiple different forms (e.g., alleles).

As those skilled in the art will appreciate, methods and compositions described herein relating to provided compounds (e.g., ds oligonucleotides) generally also apply to pharmaceutically acceptable salts of such compounds.

### DESCRIPTION OF CERTAIN EMBODIMENTS

Double stranded oligonucleotides are useful tools for a wide variety of applications. For example, ds oligonucleotides targeting HSD17B13 (e.g., NCBI Gene ID: 345275 for human HSD17B13 and related sequences from other organisms) from are useful in therapeutic, diagnostic, and research applications, including the treatment of a variety of HSD17B13-associated conditions, disorders, and diseases, including but not limited to NAFLD, NASH, ASH, alcoholic liver disease, nonalcoholic liver disease, alcoholic cirrhosis, nonalcoholic cirrhosis, steatohepatitis, hepatic steatosis, hepatoceullar carcinoma, HCV hepatitis, chronic hepatitis, hereditary hemochromatosis, primary sclerosing cholangitis, drug induced liver injury, or hepatocellular necrosis. The use of naturally occurring nucleic acids (e.g., unmodified DNA or RNA) is limited, for example, by their susceptibility to endo- and exo-nucleases. As such, various synthetic counterparts have been developed to circumvent these shortcomings and/or to further improve various properties and activities. These include synthetic oligonucleotides that contain chemical modifications, e.g., base modifications, sugar modifications, backbone modifications, etc., which, among other things, render these molecules less susceptible to degradation and improve other properties and/or activities. From a structural point of view, modifications to internucleotidic linkages can introduce chirality and/or alter charge, and certain properties may be affected by configurations of linkage phosphorus atoms of oligonucleotides. For example, binding affinity, sequence specific binding to complementary RNA, stability against nucleases, cleavage of target nucleic acids, delivery, pharmacokinetics, etc., can be affected by, inter alia, chirality and/or charge of backbone linkage atoms.

In some embodiments, a ds oligonucleotide targeting HSD17B13 comprises one or more of:
(1) a guide strand comprising backbone phosphorothioate chiral centers in Sp configuration between the 3′ terminal nucleotide and the penultimate (N-1) nucleotide and as between the penultimate (N-1) nucleotide and the immediately upstream, i.e., in the 5′ direction, (N-2) nucleotide;
(2) a guide strand comprising backbone phosphorothioate chiral centers in Rp, Sp, or alternating configurations between the 5′ terminal (+1) nucleotide and the immediately downstream, i.e., in the 3′ direction, (+2) nucleotide and between the +2 nucleotide and the immediately downstream (+3) nucleotide;
(3) a guide strand comprising one or more backbone phosphorothioate chiral centers upstream, i.e., in the 5′ direction, relative to backbone phosphorothioate chiral centers in Sp configuration between the 3′ terminal nucleotide and the penultimate (N-1) nucleotide and as between the penultimate (N-1) nucleotide and the immediately upstream (N-2) nucleotide, where the upstream backbone phosphorothioate chiral centers are in Rp or Sp configuration;
(4) a guide strand comprising one or more backbone phosphorothioate chiral centers in Rp or Sp configuration between the 5′ terminal (+1) nucleotide and the immediately downstream (+2) nucleotide and between the +2 nucleotide and the immediately downstream (+3) nucleotide, as well as between one or both of: (a) the +3 nucleotide and the +4 nucleotide; and (b) the +5 nucleotide and the +6 nucleotide;
(5) a passenger strand in combination with one or more of the aforementioned guide strands, comprising one or more backbone chiral centers in Rp or Sp configuration; and
6) a passenger strand in combination with one or more of the aforementioned guide strands, comprising backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N-1) nucleotide;
   wherein the ds oligonucleotide further comprises one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In some embodiments, a ds oligonucleotide targeting HSD17B13 comprises: (1) a phosphorothioate chiral center in Rp or Sp configuration; (2) an Rp, Sp, or stereorandom non-negatively charged internucleotidic linkage where the 3′ nucleotide of a nucleotide pair linked by an Rp, Sp, or stereorandom non-negatively charged internucleotidic linkage comprises a 2′ modification, e.g., a 2′ F; and (3) a 5′ terminal modification selected from:
(a) 5′ PO modifications, such as, but not limited to:

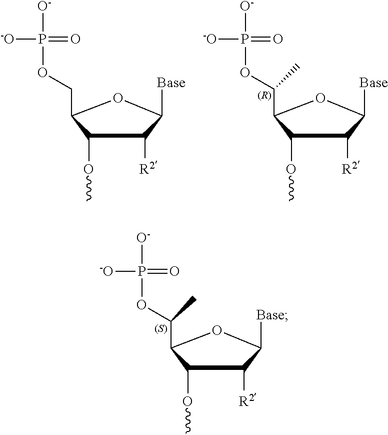

(b) 5′ VP modifications, such as, but not limited to:

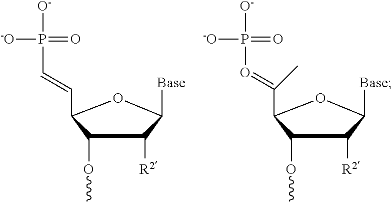

(c) 5′ MeP modifications, such as, but not limited to:

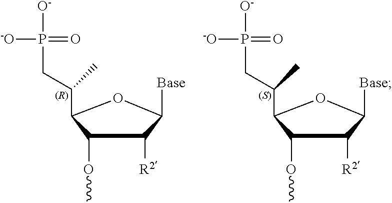

(d) 5′ PN and 5′ Trizole-P modifications, such as, but not limited to:

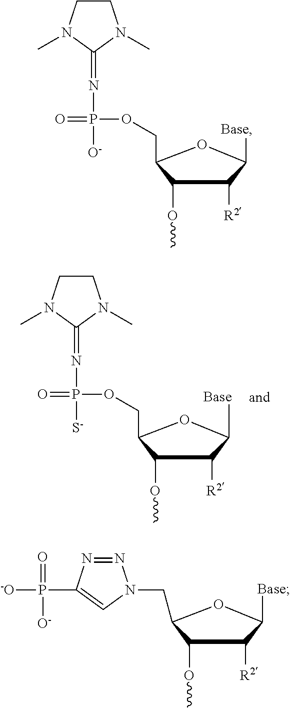

wherein Base is selected from A, C, G, T, U, abasic and modified nucleobases;
R^{2′} is selected from H, OH, O-alkyl, F, MOE, locked nucleic acid (LNA) bridges and bridged nucleic acid (BNA) bridges to the 4′ C, such as, but not limited to:

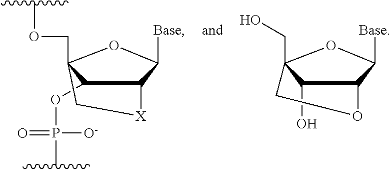

In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In some embodiments, a ds oligonucleotide targeting HSD17B13 comprises: (1) a phosphorothioate chiral center in Rp or Sp configuration; (2) an Rp, Sp, or stereorandom non-negatively charged internucleotidic linkage where the 3′ nucleotide of a nucleotide pair linked by an Rp, Sp, or stereorandom non-negatively charged internucleotidic linkage comprises a 2′ modification, e.g., a 2′ F; and (3) a 5′ terminal modification selected from:
(a) 5′ PO nucleotides, such as, but not limited to:

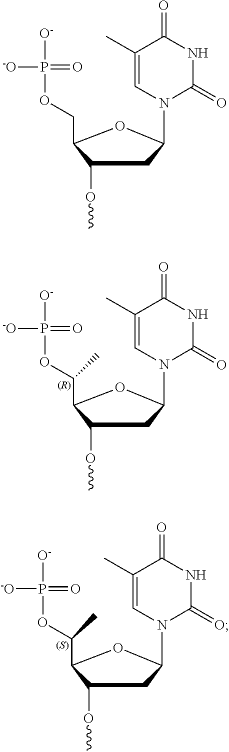

(b) 5′ VP nucleotides, such as, but not limited to:

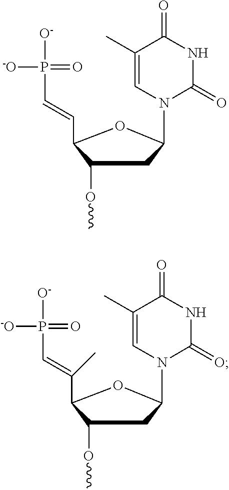

(c) 5′ MeP nucleotides, such as, but not limited to:

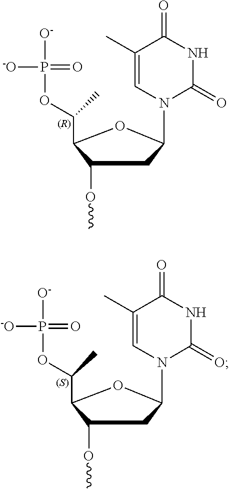

(d) 5′ PN and 5′ Trizole-P nucleotides, such as, but not limited to:

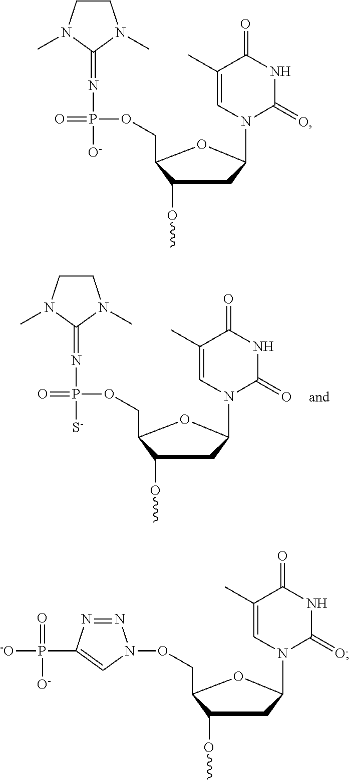

(e) 5′ abasic VP and 5′ abasic MeP nucleotides, such as, but not limited to:

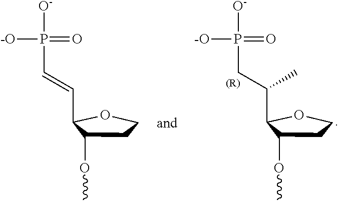

In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In some embodiments, a ds oligonucleotide targeting HSD17B13 comprises one or more of:
(1) a guide strand comprising backbone phosphorothioate chiral centers in Sp configuration between the 3′ terminal nucleotide and the penultimate (N-1) nucleotide and as between the penultimate (N-1) nucleotide and the immediately upstream, i.e., in the 5′ direction, (N-2) nucleotide;
(2) a guide strand comprising backbone phosphorothioate chiral centers in Rp, Sp, or alternating configurations between the 5′ terminal (+1) nucleotide and the immediately downstream, i.e., in the 3′ direction, (+2) nucleotide and between the +2 nucleotide and the immediately downstream (+3) nucleotide;
(3) a guide strand comprising one or more backbone phosphorothioate chiral centers upstream, i.e., in the 5′ direction, relative to backbone phosphorothioate chiral centers in Sp configuration between the 3′ terminal nucleotide and the penultimate (N-1) nucleotide and as between the penultimate (N-1) nucleotide and the immediately upstream (N-2) nucleotide, where the upstream backbone phosphorothioate chiral centers are in Rp or Sp configuration;
(4) a guide strand comprising one or more backbone phosphorothioate chiral centers in Rp or Sp configuration between the 5′ terminal (+1) nucleotide and the immediately downstream (+2) nucleotide and between the +2 nucleotide and the immediately downstream (+3) nucleotide, as well as between one or both of: (a) the +3 nucleotide and the +4 nucleotide; and (b) the +5 nucleotide and the +6 nucleotide;
(5) a passenger strand in combination with one or more of the aforementioned guide strands, comprising one or more backbone chiral centers in Rp or Sp configuration; and
6) a passenger strand in combination with one or more of the aforementioned guide strands, comprising backbone phosphorothioate chiral centers in the Sp configuration between the 5′ terminal (+1) nucleotide and the immediately downstream, i.e., in the 3′ direction, (+2) nucleotide and between the 3′ terminal nucleotide and the penultimate (N-1) nucleotide;
   wherein the ds oligonucleotide further comprises one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand;
(5) Passenger strand where one or more Rp, Sp, or stereorandom non-negatively charged internucleotidic linkage occurs downstream, i.e., in the 3′ direction, relative to the central nucleotide of the passenger strand, and
wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In some embodiments, a ds oligonucleotide targeting HSD17B13 comprises non-naturally occurring internucleotidic linkages, e.g., neutral internucleotidic linkages, which can, in certain embodiments, be used to link one or more molecules to the double-stranded oligonucleotides described herein. In certain embodiments, such linked molecules can facilitate targeting and/or delivery of the double-stranded oligonucleotide. For example, but not limitation, such linked molecules an include lipophilic molecules. In certain embodiments, the linked molecule is a molecule comprising one or more GalNac moieties. In certain embodiments, the linked molecule is a receptor. In certain embodiments, the linked molecule is a receptor ligand.

In certain embodiments, the present disclosure provides technologies (e.g., compounds, methods, etc.) for improving oligonucleotide stability while maintaining or increasing activity, including compositions of improved-stability oligonucleotides.

In certain embodiments, the present disclosure provides technologies for incorporating various additional chemical moieties into ds oligonucleotides. In certain embodiments, the present disclosure provides, for example, reagents and methods for introducing additional chemical moieties through nucleobases (e.g., by covalent linkage, optionally via a linker, to a site on a nucleobase).

In certain embodiments, the present disclosure provides technologies, e.g., ds oligonucleotide compositions and methods thereof, that achieve allele-specific suppression, wherein transcripts from one allele of a particular target gene is selectively knocked down relative to at least one other allele of the same gene.

Among other things, the present disclosure provides structural elements, technologies and/or features that can be incorporated into ds oligonucleotides and can impart or tune one or more properties thereof (e.g., relative to an otherwise identical ds oligonucleotide lacking the relevant technology or feature). In certain embodiments, the present disclosure documents that one or more provided technologies and/or features can usefully be incorporated into ds oligonucleotides of various sequences.

In certain embodiments, the present disclosure demonstrates that certain provided structural elements, technologies and/or features are particularly useful for ds oligonucleotides that participate in and/or direct RNAi mechanisms (e.g., RNAi agents). Regardless, however, the teachings of the present disclosure are not limited to ds oligonucleotides that participate in or operate via any particular mechanism.

In certain embodiments, the present disclosure pertains to any ds oligonucleotide, useful for any purpose, which operates through any mechanism, and which comprises any sequence, structure or format (or portion thereof) described herein.

In some embodiments, a ds oligonucleotide targeting HSD17B13 comprises backbone phosphorothioate chiral centers in Sp configuration between the 3′ terminal nucleotide and the penultimate (N-1) nucleotide and as between the penultimate (N-1) nucleotide and the immediately upstream (N-2) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In some embodiments, a ds oligonucleotide targeting HSD17B13 comprises backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In some embodiments, the present disclosure provides a ds oligonucleotide targeting HSD17B13 comprising one or more backbone phosphorothioate chiral centers in Rp or Sp configuration upstream of backbone phosphorothioate chiral centers in Sp configuration between the 3′ terminal nucleotide and the penultimate (N-1) nucleotide and as between the penultimate (N-1) nucleotide and the immediately upstream (N-2) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In some embodiments, the guide strand comprises one or more Rp, Sp, or stereorandom non-negatively charged internucleotidic linkage occurs between the second (+2) and third (+3) nucleotides, relative to the 5′ terminal nucleotide, of the guide strand and the internucleotidic linkage to the penultimate 3′ (N-1) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In some embodiments, the guide strand comprises backbone phosphorothioate chiral centers in Sp configuration between the 3′ terminal nucleotide and the penultimate (N-1) nucleotide and as between the penultimate (N-1) nucleotide and the immediately upstream (N-2) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone phosphorothioate chiral centers in Rp or Sp configuration. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In some embodiments, the guide strand comprises backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone chiral centers in Rp or Sp configuration. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage. In certain of the embodiments disclosed herein, the backbone phosphorothioate chiral centers are in Rp configurations between the 5′ terminal (+1) nucleotide and the immediately downstream (+2) nucleotide and between the +2 nucleotide and the immediately downstream (+3) nucleotide. In certain embodiments described herein, the backbone phosphorothioate chiral centers are in Sp configurations between the 5′ terminal (+1) nucleotide and the immediately downstream (+2) nucleotide and between the +2 nucleotide and the immediately downstream (+3) nucleotide. In certain embodiments, backbone phosphorothioate chiral centers in Rp and Sp configurations, respectively, between the 5′ terminal (+1) nucleotide and the immediately downstream (+2) nucleotide and between the +2 nucleotide and the immediately downstream (+3) nucleotide. In certain embodiments described herein, the backbone phosphorothioate chiral centers are in Sp, Rp configurations, respectively, between the 5′ terminal (+1) nucleotide and the immediately downstream (+2) nucleotide and between the +2 nucleotide and the immediately downstream (+3) nucleotide.

In some embodiments, the guide strand comprises one or more backbone phosphorothioate chiral centers in Rp or Sp configuration upstream of backbone chiral centers in Sp configuration between the 3′ terminal nucleotide and the penultimate (N-1) nucleotide and as between the penultimate (N-1) nucleotide and the immediately upstream (N-2) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone chiral centers in Rp or Sp configuration. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In some embodiments, the guide strand comprises one or more backbone phosphorothioate chiral centers in Rp or Sp configuration between the 5′ terminal (+1) nucleotide and the immediately downstream (+2) nucleotide and between the (+2) nucleotide and the immediately downstream (+3) nucleotide, as well as between one or both of: (a) the (+3) nucleotide and the (+4) nucleotide; and (b) the (+5) nucleotide and the (+6) nucleotide, and one or more of:
(1) a guide strand where one or both of the 5′ and 3′ terminal dinucleotides are not linked by non-negatively charged internucleotidic linkages, i.e., the guide strand comprises one more non-negatively charged internucleotidic linkages downstream, i.e., in the 3′ direction, relative to the linkage between the 5′ terminal dinucleotide and/or upstream, i.e., in the 5′ direction, relative to the linkage between the 3′ terminal dinucleotide;
(2) a guide strand where one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
(3) a guide strand where an Rp, Sp, or stereorandom non-negatively charged internucleotidic linkage occurs between the third (+3) and fourth (+4) nucleotides, relative to the 5′ terminal nucleotide, of the guide strand and/or between the tenth (+10) and eleventh (+11) nucleotides, relative to the 5′ terminal nucleotide;
(4) a passenger strand where one or more Rp, Sp, or stereorandom non-negatively charged internucleotidic linkage occurs upstream, i.e., in the 5′ direction, relative to the central nucleotide of the passenger strand; and
(5) Passenger strand where one or more Rp, Sp, or stereorandom non-negatively charged internucleotidic linkage occurs downstream, i.e., in the 3′ direction, relative to the central nucleotide of the passenger strand, and
   wherein the ds oligonucleotide further comprises a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone chiral centers in Rp or Sp configuration. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In some embodiments, the guide strand comprises one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises one or more backbone chiral centers in Rp or Sp configuration.

In certain embodiments, the guide strand comprises backbone phosphorothioate chiral centers in Sp configuration between the 3′ terminal nucleotide and the penultimate (N-1) nucleotide and as between the penultimate (N-1) nucleotide and the immediately upstream (N-2) nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49 and one or more backbone chiral centers in Rp or Sp configuration.

In certain embodiments, the guide strand comprises backbone phosphorothioate chiral centers in Rp, Sp, or alternating configurations between the 5′ terminal (+1) nucleotide and the immediately downstream (+2) nucleotide and between the +2 nucleotide and the immediately downstream (+3) nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49 and one or more backbone chiral centers in Rp or Sp configuration. In certain embodiments, the one or more Rp, Sp, or stereorandom non-negatively charged internucleotidic linkage incorporated into the guide or passenger strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain embodiments, the guide strand comprises one or more backbone phosphorothioate chiral centers in Rp or Sp configuration upstream of backbone phosphorothioate chiral centers in Sp configuration between the 3′ terminal nucleotide and the penultimate (N-1) nucleotide and as between the penultimate (N-1) nucleotide and the immediately upstream (N-2) nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49 and one or more backbone chiral centers in Rp or Sp configuration. In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain embodiments, the guide strand comprises one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide, a 2′ modification, e.g., a 2′ F modification, of the 3′ nucleotide of a nucleotide pair linked by an Rp, Sp, or stereorandom non-negatively charged internucleotidic linkage, and the passenger strand comprises 0-n Rp, Sp, or stereorandom non-negatively charged internucleotidic linkages, where n is about 1 to 49 and one or more backbone chiral centers in Rp or Sp configuration. In certain embodiments, the one or more Rp, Sp, or stereorandom non-negatively charged internucleotidic linkage incorporated into the guide strand is an Rp non-negatively charged internucleotidic linkage. In certain embodiments, the one or more Rp, Sp, or stereorandom non-negatively charged internucleotidic linkage is an Sp non-negatively charged internucleotidic linkage. In certain embodiments, the one or more Rp, Sp, or stereorandom non-negatively charged internucleotidic linkage is a stereorandom non-negatively charged internucleotidic linkage.

In certain embodiments, internucleotidic linkages of an oligonucleotide comprise or consist of 1-5, 1-10, 1-15, 1-20, 1-25, 1-30, 1-40, 1-50, or 1, 2, 3, 4, 5, 6, 7, 8, 9, 10, 11, 12, 13, 14, 15, 16, 17, 18, 19, 20 or more chirally controlled internucleotidic linkages. In certain embodiments, the present disclosure provides a dsRNAi oligonucleotide composition wherein the dsRNAi oligonucleotides comprise at least one chirally controlled internucleotidic linkage.

In certain embodiments, the present disclosure provides a dsRNAi oligonucleotide composition wherein the dsRNAi oligonucleotides are stereorandom or not chirally controlled. In certain embodiments, in a dsRNAi oligonucleotide, at least one internucleotidic linkage is stereorandom and at least one internucleotidic linkage is chirally controlled.

In certain embodiments, internucleotidic linkages of an oligonucleotide comprise or consist of one or more neutrally charged internucleotidic linkages.

### HSD17B13

In some embodiments, HSD17B13 refers to a gene or a gene product thereof (including but not limited to, a nucleic acid, including but not limited to a DNA or RNA, a transcript, a protein encoded thereby; can be from any form of HSD17B13, e.g., wide-type or mutant alleles) from any species, which may be known as: ****. In some embodiments, it refers to the gene and product thereof in human. In some embodiments, it refers to the gene and product thereof in a non-human primate. Various HSD17B13 sequences, including variants thereof, from human, mouse, rat, monkey, etc., are readily available to those of skill in the art. In some embodiments, HSD17B13 is a human or mouse HSD17B13, which is wild-type or mutant. It has been reported that HSD17B13 can have a number of functions. Various technologies, e.g., assays, cells, animal models, etc., have also been reported and can be utilized for characterization and/or assessment of provided technologies (e.g., oligonucleotides, compositions, methods, etc.) in accordance with the present disclosure.

In some embodiments, a HSD17B13 gene, transcript (e.g., mRNA before or after splicing), or protein variant or isoform comprises a mutation. In some embodiments, a HSD17B13 gene, transcript or protein is or a transcription or translation product of an alternatively spliced variant or isoform.

### HSD17B13-Associated Conditions, Disorder, or Diseases

Various conditions, disorders, or diseases are reported to be associated with HSD17B13. Generally, a disease, disorder, or condition is associated with HSD17B13 if the presence, level, activity, and/or form of HSD17B13 and/or products (e.g., transcripts, encoded proteins, etc.) thereof correlates with incidence of and/or susceptibility to the disease, disorder, or condition (e.g., across a relevant population). In some embodiments, a condition, disorder, or disease associated with HSD17B13 may be treated and/or prevented by reducing expression, level and/or activity of HSD17B13 transcripts and/or proteins.

Various HSD17B13-associated conditions, disorders, or diseases are reported. In some embodiments, a HSD17B13-associated condition, disorder, or disease is NAFLD, NASH, ASH, alcoholic liver disease, nonalcoholic liver disease, alcoholic cirrhosis, nonalcoholic cirrhosis, steatohepatitis, hepatic steatosis, hepatoceullar carcinoma, HCV hepatitis, chronic hepatitis, hereditary hemochromatosis, primary sclerosing cholangitis, drug induced liver injury, or hepatocellular necrosis. In some embodiments, a HSD17B13-associated condition, disorder, or disease is NAFLD. In some embodiments, a HSD17B13-associated condition, disorder, or disease is NASH.

Among other things, provided technologies are useful for treating or preventing a condition, disorder, or disease associated with HSD17B13, e.g., NAFLD, NASH, ASH, alcoholic liver disease, nonalcoholic liver disease, alcoholic cirrhosis, nonalcoholic cirrhosis, steatohepatitis, hepatic steatosis, hepatoceullar carcinoma, HCV hepatitis, chronic hepatitis, hereditary hemochromatosis, primary sclerosing cholangitis, drug induced liver injury, or hepatocellular necrosis etc. In some embodiments, the present disclosure pertains to the use of a ds oligonucleotide targeting HSD17B13 or a composition thereof in the treatment of a HSD17B13-associated disorder, disease or condition, e.g., NAFLD, NASH, ASH, alcoholic liver disease, nonalcoholic liver disease, alcoholic cirrhosis, nonalcoholic cirrhosis, steatohepatitis, hepatic steatosis, hepatoceullar carcinoma, HCV hepatitis, chronic hepatitis, hereditary hemochromatosis, primary sclerosing cholangitis, drug induced liver injury, or hepatocellular necrosis, etc.

In some embodiments, treatment or prevention with provided technologies reduces rate of HSD17B13 production and reduces or halts or reverses accumulation of HSD17B13. In some embodiments, treatment or prevention with provided technologies reduces the rate of clinical decline, or delays or prevents onset of a condition, disorder, or disease.

As appreciated by those skilled in the art, mechanisms, genotypes, symptoms, biomarkers, etc. of such conditions, disorders, or diseases may be utilized in accordance with the present disclosure to characterize/assess provided technologies.

### Double Stranded Oligonucleotides

Among other things, the present disclosure provides ds oligonucleotides of various designs, which may comprise various nucleobases and patterns thereof, sugars and patterns thereof, internucleotidic linkages and patterns thereof, and/or additional chemical moieties and patterns thereof as described in the present disclosure. In some embodiments, provided ds oligonucleotides targeting HSD17B13 can direct a decrease in the expression, level and/or activity of a HSD17B13 gene and/or one or more of its products (e.g., transcripts, mRNA, proteins, etc.). In some embodiments, provided ds oligonucleotides targeting HSD17B13 can direct a decrease in the expression, level and/or activity of a HSD17B13 gene and/or one or more of its products in a cell of a subject or patient. In some embodiments, a cell normally expresses HSD17B13 or produces HSD17B13 protein. In some embodiments, provided ds oligonucleotides targeting HSD17B13 can direct a decrease in the expression, level and/or activity of a HSD17B13 target gene or a gene product and has a base sequence which consists of, comprises, or comprises a portion (e.g., a span of 10, 11, 12, 13, 14, 15, 16, 17, 18, 19, 20 or more contiguous bases) of the base sequence of a ds oligonucleotide targeting HSD17B13 disclosed herein, wherein each T can be independently substituted with U and vice versa, and the ds oligonucleotide comprises at least one non-naturally-occurring modification of a base, sugar and/or internucleotidic linkage.

In some embodiments, ds oligonucleotides targeting HSD17B13 can direct a decrease in the expression, level and/or activity of a target gene, e.g., a HSD17B13 target gene, or a product thereof. In some embodiments, ds oligonucleotides targeting HSD17B13 can direct a decrease in the expression, level and/or activity of a HSD17B13 target gene or a product thereof via RNase H-mediated knockdown. In some embodiments, ds oligonucleotides targeting HSD17B13 can direct a decrease in the expression, level and/or activity of a HSD17B13 target gene or a product thereof by sterically blocking translation after binding to a HSD17B13 target gene mRNA, and/or by altering or interfering with mRNA splicing. Regardless, however, the present disclosure is not limited to any particular mechanism. In some embodiments, the present disclosure provides oligonucleotides, compositions, methods, etc., capable of operating via double-stranded RNA interference.

In some embodiments, a ds oligonucleotide targeting HSD17B13 is capable of mediating a decrease in the expression, level and/or activity of HSD17B13. In some embodiments, a ds oligonucleotide targeting HSD17B13 is capable of mediating a decrease in the level of HSD17B13 proteins. In some embodiments, a ds oligonucleotide targeting HSD17B13 is capable of mediating a decrease in the level of HSD17B13 proteins.

In some embodiments, a ds oligonucleotide targeting HSD17B13 is capable of mediating a decrease in the expression, level and/or activity of HSD17B13 via a mechanism involving mRNA degradation.

In some embodiments, a ds oligonucleotide targeting HSD17B13 is capable of mediating a decrease in the expression, level and/or activity of more than one HSD17B13 allele.

In some embodiments, the present disclosure pertains to a method of treatment of a HSD17B13-associated disease, disorder or condition, wherein HSD17B13 is expressed, comprising the step of administering a therapeutically effective amount of a ds oligonucleotide targeting HSD17B13 capable of mediating a decrease in the expression, level and/or activity of HSD17B13. In some embodiments, multiple forms, e.g., alleles, of HSD17B13 may exist, and provided technologies can reduce expression, level and/or activity of two or more or all of the forms and products thereof.

In some embodiments, the present disclosure pertains to a method of treatment of a HSD17B13-associated disease, disorder or condition, comprising the step of administering a therapeutic amount of a ds oligonucleotide targeting HSD17B13 capable of mediating a decrease in the expression, level and/or activity of HSD17B13.

In some embodiments, a ds oligonucleotide targeting HSD17B13 is capable of mediating a decrease in the expression, level and/or activity of HSD17B13 via a mechanism involving splicing modulation, e.g., exon skipping.

In some embodiments, a ds oligonucleotide targeting HSD17B13 comprises a structural element or a portion thereof described herein, e.g., in Table 1. In some embodiments, a ds oligonucleotide targeting HSD17B13 comprises a base sequence (or a portion thereof) described herein, wherein each T can be independently substituted with U and vice versa, a chemical modification or a pattern of chemical modifications (or a portion thereof), and/or a format or a portion thereof described herein. In some embodiments, a ds oligonucleotide targeting HSD17B13 has a base sequence which comprises the base sequence (or a portion thereof) wherein each T can be independently substituted with U, pattern of chemical modifications (or a portion thereof), and/or a format of an oligonucleotide disclosed herein, e.g., in Table 1, or otherwise disclosed herein. In some embodiments, such oligonucleotides, e.g., ds oligonucleotides targeting HSD17B13, reduce expression, level and/or activity of a gene, e.g., a HSD17B13 gene, or a gene product thereof.

Among other things, ds oligonucleotides targeting HSD17B13 can hybridize to their target nucleic acids (e.g., pre-mRNA, mature mRNA, etc.). For example, in some embodiments, a ds oligonucleotide targeting HSD17B13 can hybridize to a HSD17B13 nucleic acid derived from a DNA strand (either strand of the HSD17B13 gene). In some embodiments, a ds oligonucleotide targeting HSD17B13 can hybridize to a HSD17B13 transcript. In some embodiments, a ds oligonucleotide targeting HSD17B13 can hybridize to a HSD17B13 nucleic acid in any stage of RNA processing, including but not limited to a pre-mRNA or a mature mRNA. In some embodiments, a ds oligonucleotide targeting HSD17B13 can hybridize to any element of a HSD17B13 nucleic acid or its complement, including but not limited to: a promoter region, an enhancer region, a transcriptional stop region, a translational start signal, a translation stop signal, a coding region, a non-coding region, an exon, an intron, an intron/exon or exon/intron junction, the 5′ UTR, or the 3′ UTR. In some embodiments, ds oligonucleotides targeting HSD17B13 can hybridize to their targets with no more than 2 mismatches. In some embodiments, ds oligonucleotides targeting HSD17B13 can hybridize to their targets with no more than one mismatch. In some embodiments, ds oligonucleotides targeting HSD17B13 can hybridize to their targets with no mismatches (e.g., when all C-G and/or A-T/U base paring).

In some embodiments, an oligonucleotide can hybridize to two or more variants of transcripts. In some embodiments, a ds oligonucleotide targeting HSD17B13 can hybridize to two or more or all variants of HSD17B13 transcripts. In some embodiments, a ds oligonucleotide targeting HSD17B13 can hybridize to two or more or all variants of HSD17B13 transcripts derived from the sense strand.

In some embodiments, a HSD17B13 target of a ds oligonucleotide targeting HSD17B13 is a HSD17B13 RNA which is not a mRNA.

In some embodiments, oligonucleotides, e.g., ds oligonucleotides targeting HSD17B13, contain increased levels of one or more isotopes. In some embodiments, oligonucleotides, e.g., ds oligonucleotides targeting HSD17B13, are labeled, e.g., by one or more isotopes of one or more elements, e.g., hydrogen, carbon, nitrogen, etc. In some embodiments, oligonucleotides, e.g., ds oligonucleotides targeting HSD17B13, in provided compositions, e.g., oligonucleotides of a plurality of a composition, comprise base modifications, sugar modifications, and/or internucleotidic linkage modifications, wherein the oligonucleotides contain an enriched level of deuterium. In some embodiments, oligonucleotides, e.g., ds oligonucleotides targeting HSD17B13, are labeled with deuterium (replacing -¹H with -²H) at one or more positions. In some embodiments, one or more ¹H of an oligonucleotide chain or any moiety conjugated to the oligonucleotide chain (e.g., a targeting moiety, etc.) is substituted with ²H. Such oligonucleotides can be used in compositions and methods described herein.

In some embodiments, the present disclosure provides an oligonucleotide composition comprising a plurality of oligonucleotides which:
1) have a common base sequence complementary to a target sequence (e.g., a HSD17B13 target sequence) in a transcript; and
2) comprise one or more modified sugar moieties and/or modified internucleotidic linkages.

In some embodiments, ds oligonucleotides targeting HSD17B13 having a common base sequence may have the same pattern of nucleoside modifications, e.g., sugar modifications, base modifications, etc. In some embodiments, a pattern of nucleoside modifications may be represented by a combination of locations and modifications. In some embodiments, a pattern of backbone linkages comprises locations and types (e.g., phosphate, phosphorothioate, substituted phosphorothioate, etc.) of each internucleotidic linkage.

In some embodiments, oligonucleotides of a plurality, e.g., in provided compositions, are of the same oligonucleotide type. In some embodiments, oligonucleotides of an oligonucleotide type have a common pattern of sugar modifications. In some embodiments, oligonucleotides of an oligonucleotide type have a common pattern of base modifications. In some embodiments, oligonucleotides of an oligonucleotide type have a common pattern of nucleoside modifications. In some embodiments, oligonucleotides of an oligonucleotide type have the same constitution. In some embodiments, oligonucleotides of an oligonucleotide type are identical. In some embodiments, oligonucleotides of a plurality are identical. In some embodiments, oligonucleotides of a plurality share the same constitution.

In some embodiments, as exemplified herein, ds oligonucleotides targeting HSD17B13 are chiral controlled, comprising one or more chirally controlled internucleotidic linkages. In some embodiments, ds oligonucleotides targeting HSD17B13 are stereochemically pure. In some embodiments, ds oligonucleotides targeting HSD17B13 are substantially separated from other stereoisomers.

In some embodiments, ds oligonucleotides targeting HSD17B13 comprise one or more modified nucleobases, one or more modified sugars, and/or one or more modified internucleotidic linkages.

In some embodiments, ds oligonucleotides targeting HSD17B13 comprise one or more modified sugars. In some embodiments, oligonucleotides of the present disclosure comprise one or more modified nucleobases. Various modifications can be introduced to a sugar and/or nucleobase in accordance with the present disclosure. For example, in some embodiments, a modification is a modification described in U.S. Pat. No. 9,006,198. In some embodiments, a modification is a modification described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, and/or WO 2020/191252, the sugar, base, and internucleotidic linkage modifications of each of which are independently incorporated herein by reference.

As used in the present disclosure, in some embodiments, “one or more” is 1-200, 1-150, 1-100, 1-90, 1-80, 1-70, 1-60, 1-50, 1-40, 1-30, or 1, 2, 3, 4, 5, 6, 7, 8, 9, 10, 11, 12, 13, 14, 15, 16, 17, 18, 19, 20, 21, 22, 23, 24, or 25. In some embodiments, “one or more” is one. In some embodiments, “one or more” is two. In some embodiments, “one or more” is three. In some embodiments, “one or more” is four. In some embodiments, “one or more” is five. In some embodiments, “one or more” is six. In some embodiments, “one or more” is seven. In some embodiments, “one or more” is eight. In some embodiments, “one or more” is nine. In some embodiments, “one or more” is ten. In some embodiments, “one or more” is at least one. In some embodiments, “one or more” is at least two. In some embodiments, “one or more” is at least three. In some embodiments, “one or more” is at least four. In some embodiments, “one or more” is at least five. In some embodiments, “one or more” is at least six. In some embodiments, “one or more” is at least seven. In some embodiments, “one or more” is at least eight. In some embodiments, “one or more” is at least nine. In some embodiments, “one or more” is at least ten.

As used in the present disclosure, in some embodiments, “at least one” is 1-200, 1-150, 1-100, 1-90, 1-80, 1-70, 1-60, 1-50, 1-40, 1-30, or 1, 2, 3, 4, 5, 6, 7, 8, 9, 10, 11, 12, 13, 14, 15, 16, 17, 18, 19, 20, 21, 22, 23, 24, or 25. In some embodiments, “at least one” is one. In some embodiments, “at least one” is two. In some embodiments, “at least one” is three. In some embodiments, “at least one” is four. In some embodiments, “at least one” is five. In some embodiments, “at least one” is six. In some embodiments, “at least one” is seven. In some embodiments, “at least one” is eight. In some embodiments, “at least one” is nine. In some embodiments, “at least one” is ten.

In some embodiments, a ds oligonucleotide targeting HSD17B13 is or comprises a ds oligonucleotide targeting HSD17B13 described in Table 1.

As demonstrated in the present disclosure, in some embodiments, a provided oligonucleotide (e.g., a ds oligonucleotide targeting HSD17B13) is characterized in that, when it is contacted with the transcript in a knockdown system, knockdown of its target (e.g., a HSD17B13 transcript for a ds oligonucleotide targeting HSD17B13) is achieved.

In some embodiments, ds oligonucleotides are provided as salt forms. In some embodiments, ds oligonucleotides are provided as salts comprising negatively-charged internucleotidic linkages (e.g., phosphorothioate internucleotidic linkages, natural phosphate linkages, etc.) existing as their salt forms. In some embodiments, ds oligonucleotides are provided as pharmaceutically acceptable salts. In some embodiments, ds oligonucleotides are provided as metal salts. In some embodiments, oligonucleotides are provided as sodium salts. In some embodiments, ds oligonucleotides are provided as metal salts, e.g., sodium salts, wherein each negatively-charged internucleotidic linkage is independently in a salt form (e.g., for sodium salts, —O—P(O)(SNa)—O— for a phosphorothioate internucleotidic linkage, —O—P(O)(ONa)—O— for a natural phosphate linkage, etc.).

### Double Stranded Oligonucleotide Base Sequences

In some embodiments, a ds oligonucleotide targeting HSD17B13 comprises a base sequence described herein or a portion (e.g., a span of 5-50, 5-40, 5-30, 5-20, or 5, 6, 7, 8, 9, 10, 11, 12, 13, 14, 15, 16, 17, 18, 19, 20, 21, 22, 23, 24, 25, 26, 27, 28, 30 or at least 10, at least 15, contiguous nucleobases) thereof with 0-5 (e.g., 0, 1, 2, 3, 4 or 5) mismatches, wherein each T can be independently substituted with U and vice versa. In some embodiments, a ds oligonucleotide targeting HSD17B13 comprises a base sequence described herein, or a portion thereof, wherein a portion is a span of at least 10 contiguous nucleobases, or a span of at least 15 contiguous nucleobases with 1-5 mismatches. In some embodiments, ds oligonucleotides targeting HSD17B13 comprise a base sequence described herein, or a portion thereof, wherein a portion is a span of at least 10 contiguous nucleobases, or a span of at least 10 contiguous nucleobases with 1-5 mismatches, wherein each T can be independently substituted with U and vice versa. In some embodiments, base sequences of oligonucleotides comprise or consists of 10-50 (e.g., about or at least 10, 11, 12, 13, 14, 15, 16, 17, 18, 19, 20, 21, 22, 23, 24, 25, 30, 35, 40, 45; in some embodiments, at least 15; in some embodiments, at least 16; in some embodiments, at least 17; in some embodiments, at least 18; in some embodiments, at least 19; in some embodiments, at least 20; in some embodiments, at least 21; in some embodiments, at least 22; in some embodiments, at least 23; in some embodiments, at least 24; in some embodiments, at least 25) contiguous bases of a base sequence that is identical to or complementary to a base sequence of a HSD17B13 gene or a transcript (e.g., mRNA) thereof (e.g., in an intron).

Base sequences of ds oligonucleotides targeting HSD17B13, as appreciated by those skilled in the art, typically have sufficient length and complementarity to their targets, e.g., RNA transcripts (e.g., pre-mRNA, mature mRNA, etc.) to mediate target-specific knockdown. In some embodiments, the base sequence of a ds oligonucleotide targeting HSD17B13 has a sufficient length and identity to a HSD17B13 transcript target to mediate target-specific knockdown. In some embodiments, the ds oligonucleotide targeting HSD17B13 is complementary to a portion of a HSD17B13 transcript (a HSD17B13 transcript target sequence). In some embodiments, the base sequence of a ds oligonucleotide targeting HSD17B13 has 90% or more identity with the base sequence of an oligonucleotide disclosed in Table 1, wherein each T can be independently substituted with U and vice versa. In some embodiments, the base sequence of a ds oligonucleotide targeting HSD17B13 has 95% or more identity with the base sequence of an oligonucleotide disclosed in Table 1, wherein each T can be independently substituted with U and vice versa. In some embodiments, the base sequence of a ds oligonucleotide targeting HSD17B13 comprises a continuous span of 15 or more bases of an oligonucleotide disclosed in Table 1, wherein each T can be independently substituted with U and vice versa, except that one or more bases within the span are abasic (e.g., a nucleobase is absent from a nucleotide). In some embodiments, the base sequence of a ds oligonucleotide targeting HSD17B13 comprises a continuous span of 19 or more bases of a ds oligonucleotide targeting HSD17B13 disclosed herein, except that one or more bases within the span are abasic (e.g., a nucleobase is absent from a nucleotide). In some embodiments, the base sequence of a ds oligonucleotide targeting HSD17B13 comprises a continuous span of 19 or more bases of an oligonucleotide disclosed herein, wherein each T can be independently substituted with U and vice versa, except for a difference in the 1 or 2 bases at the 5′ end and/or 3′ end of the base sequences.

In some embodiments, the present disclosure pertains to an oligonucleotide having a base sequence which comprises the base sequence of any oligonucleotide disclosed herein, wherein each T may be independently replaced with U and vice versa.

In some embodiments, the present disclosure pertains to an oligonucleotide having a base sequence which comprises at least 15 contiguous bases of the base sequence of any oligonucleotide disclosed herein, wherein each T may be independently replaced with U and vice versa.

In some embodiments, the present disclosure pertains to an oligonucleotide having a base sequence which is at least 90% identical to the base sequence of any oligonucleotide disclosed herein, wherein each T may be independently replaced with U and vice versa.

In some embodiments, the present disclosure pertains to an oligonucleotide having a base sequence which is at least 95% identical to the base sequence of any oligonucleotide disclosed herein, wherein each T may be independently replaced with U and vice versa.

In some embodiments, a ds oligonucleotide targeting HSD17B13 is selected from Table 1.

In some embodiments, the base sequence of a ds oligonucleotide targeting HSD17B13 is complementary to that of a HSD17B13 transcript or a portion thereof.

In some embodiments, the base sequence of a ds oligonucleotide targeting HSD17B13 is complementary to a portion of a HSD17B13 nucleic acid sequence, e.g., a HSD17B13 gene sequence, a HSD17B13 transcript, a HSD17B13 mRNA sequence, etc. In some embodiments, ads oligonucleotide targeting HSD17B13 is identical to a portion of a HSD17B13 nucleic acid sequence, e.g., a HSD17B13 gene sequence, a HSD17B13 transcript, a HSD17B13 mRNA sequence, etc. In some embodiments, the base sequence of such a portion is characteristic of HSD17B13 in that no other genomic or transcript sequences in a system contain the same sequence as the portion. In some embodiments, no other genomic or transcript sequences in a system contain a sequence that differs from such a portion at no more than 1 nucleobase. In some embodiments, no other genomic or transcript sequences in a system contain a sequence that differs from such a portion at no more than 2 nucleobases. In some embodiments, a portion of a gene that is complementary to an oligonucleotide is referred to as a target sequence of the oligonucleotide. In some embodiments, a system is or comprises a cell, sample, tissue, organ, or a species. For example, for oligonucleotides targeting human HSD17B13, a relevant species in many embodiments is human. In some embodiments, a system can be or comprises multiple species, e.g., when cross-species activities and/or properties are characterized and/or assessed. In some embodiments, such a portion is in an exon. In some embodiments, such a portion is in an intron. In some embodiments, such a portion spans an intron and an exon. In some embodiments, such a portion spans two exons. In some embodiments, such a portion is in a 5′-UTR region. In some embodiments, such a portion is in a 3′-UTR region.

In some embodiments, a ds oligonucleotide targeting HSD17B13 targets two or more or all alleles (if multiple alleles exist in a relevant system) of HSD17B13. In some embodiments, an oligonucleotide reduces expressions, levels and/or activities of both wild-type HSD17B13 and mutant HSD17B13, and/or transcripts and/or products thereof.

In some embodiments, base sequences of provided oligonucleotides are fully complementary to both human and a non-human primate (NHP) HSD17B13 target sequences. In some embodiments, such sequences can be particularly useful as they can be readily assessed in both human and non-human primates.

In some embodiments, a ds oligonucleotide targeting HSD17B13 comprises a base sequence or portion thereof described in the Tables, wherein each T may be independently replaced with U and vice versa, and/or a sugar, nucleobase, and/or internucleotidic linkage modification and/or a pattern thereof described in Table 1, and/or an additional chemical moiety (in addition to an oligonucleotide chain, e.g., a target moiety, a lipid moiety, a carbohydrate moiety, etc.) described in Table 1.

In some embodiments, the terms “complementary,” “fully complementary” and “substantially complementary” may be used with respect to the base matching between n oligonucleotide (e.g., a ds oligonucleotide targeting HSD17B13) base sequence and a target sequence (e.g., a HSD17B13 target sequence), as will be understood by those skilled in the art from the context of their use. It is noted that substitution of T for U, or vice versa, generally does not alter the amount of complementarity. As used herein, an oligonucleotide that is “substantially complementary” to a target sequence is largely or mostly complementary but not 100% complementary. In some embodiments, a sequence (e.g., a ds oligonucleotide targeting HSD17B13) which is substantially complementary has 1, 2, 3, 4 or 5 mismatches when aligned to its target sequence. In some embodiments, ads oligonucleotide targeting HSD17B13 has a base sequence which is substantially complementary to a HSD17B13 target sequence. In some embodiments, a ds oligonucleotide targeting HSD17B13 has a base sequence which is substantially complementary to the complement of the sequence of a ds oligonucleotide targeting HSD17B13 disclosed herein. As appreciated by those skilled in the art, in some embodiments, sequences of oligonucleotides need not be 100% complementary to their targets for the oligonucleotides to perform their functions (e.g., knockdown of target nucleic acids. Typically, when determining complementarity, A and T (or U) are complementary nucleobases and C and G are complementary nucleobases.

In some embodiments, the present disclosure provides a ds oligonucleotide targeting HSD17B13 comprising a sequence found in an oligonucleotide described in a Table. In some embodiments, the present disclosure provides a ds oligonucleotide targeting HSD17B13 comprising a sequence found in an oligonucleotide described in Table 1, wherein one or more U is independently and optionally replaced with T or vice versa. In some embodiments, a ds oligonucleotide targeting HSD17B13 can comprise at least one T and/or at least one U. In some embodiments, the present disclosure provides a ds oligonucleotide targeting HSD17B13 comprising a sequence found in an oligonucleotide described in a Table, wherein the said sequence has over 50% identity with the sequence of the oligonucleotide described in the Table. In some embodiments, the present disclosure provides a ds oligonucleotide targeting HSD17B13 comprising the sequence of an oligonucleotide disclosed in Table 1. In some embodiments, the present disclosure provides a ds oligonucleotide targeting HSD17B13 whose base sequence is the sequence of an oligonucleotide disclosed in Table 1, wherein each T may be independently replaced with U and vice versa. In some embodiments, the present disclosure provides a ds oligonucleotide targeting HSD17B13 comprising a sequence found in an oligonucleotide in Table 1, wherein the oligonucleotides have a pattern of backbone linkages, pattern of backbone chiral centers, and/or pattern of backbone phosphorus modifications of the same oligonucleotide or another oligonucleotide in Table 1.

Among other things, the present disclosure presents, in Table 1 and elsewhere, various ds oligonucleotides, each of which has a defined base sequence. In some embodiments, the present disclosure, the present disclosure provides an oligonucleotide whose base sequence which is, comprises, or comprises a portion of the base sequence of an oligonucleotide disclosed herein, e.g., in Table 1 herein, wherein each T may be independently replaced with U and vice versa. In some embodiments, the disclosure provides an oligonucleotide having a base sequence which is, comprises, or comprises a portion of the base sequence of an oligonucleotide disclosed herein, e.g., in Table 1, wherein each T may be independently replaced with U and vice versa, wherein the oligonucleotide further comprises a chemical modification, stereochemistry, format, an additional chemical moiety described herein (e.g., a targeting moiety, lipid moiety, carbohydrate moiety, etc.), and/or another structural feature.

In some embodiments, a “portion” (e.g., of a base sequence or a pattern of modifications) is at least 5, 6, 7, 8, 9, 10, 11, 12, 13, 14, 15, 16, 17, 18, 19, or 20 monomeric units long (e.g., for a base sequence, at least 5, 6, 7, 8, 9, 10, 11, 12, 13, 14, 15, 16, 17, 18, 19, or 20 bases long). In some embodiments, a “portion” of a base sequence is at least 5 bases long. In some embodiments, a “portion” of a base sequence is at least 10 bases long. In some embodiments, a “portion” of a base sequence is at least 15 bases long. In some embodiments, a “portion” of a base sequence is at least 16, 17, 18, 19 or 20 bases long. In some embodiments, a “portion” of a base sequence is at least 20 bases long. In some embodiments, a portion of a base sequence is 10, 11, 12, 13, 14, 15, 16, 17, 18, 19 or more contiguous (consecutive) bases. In some embodiments, a portion of a base sequence is 15 or more contiguous (consecutive) bases. In some embodiments, a portion of a base sequence is 16, 17, 18, 19 or 20 or more contiguous (consecutive) bases. In some embodiments, a portion of a base sequence is 20 or more contiguous (consecutive) bases.

In some embodiments, the present disclosure provides an oligonucleotide (e.g., a ds oligonucleotide targeting HSD17B13) whose base sequence is a base sequence of an oligonucleotide in Table 1 or a portion thereof, wherein each T may be independently replaced with U and vice versa. In some embodiments, the present disclosure provides a ds oligonucleotide targeting HSD17B13 of a sequence of an oligonucleotide in Table 1, wherein the oligonucleotide is capable of directing a decrease in the expression, level and/or activity of a HSD17B13 gene or a gene product thereof. As appreciated by those skilled in the art, in provided base sequence, each U may be optionally and independently replaced by T or vice versa, and a sequence can comprise a mixture of U and T. In some embodiments, C may be optionally and independently replaced with 5mC.

In some embodiments, a portion is a span of at least 15, 16, 17, 18, 19, 20, 21, 22, 23, 24, or 25 total nucleotides. In some embodiments, a portion is a span of at least 15, 16, 17, 18, 19, 20, 21, 22, 23, 24, or 25 total nucleotides with 0-3 mismatches. In some embodiments, a portion is a span of at least 15, 16, 17, 18, 19, 20, 21, 22, 23, 24, or 25 total nucleotides with 0-3 mismatches, wherein a span with 0 mismatches is complementary and a span with 1 or more mismatches is a non-limiting example of substantial complementarity. In some embodiments, a base comprises a portion characteristic of a nucleic acid (e.g., a gene) in that the portion is identical or complementary to a portion of the nucleic acid or a transcript thereof, and is not identical or complementary to a portion of any other nucleic acid (e.g., a gene) or a transcript thereof in the same genome. In some embodiments, a portion is characteristic of human HSD17B13.

In some embodiments, a provided oligonucleotide, e.g., a ds oligonucleotide targeting HSD17B13, has a length of no more than about 49, 45, 40, 30, 35, 25, or 23 total nucleotides as described herein. In some embodiments, wherein the sequence recited herein starts with a U or T at the 5′-end, the U can be deleted and/or replaced by another base. In some embodiments, an oligonucleotide has a base sequence which is or comprises or comprises a portion of the base sequence of an oligonucleotide in a Table, wherein each T may be independently replaced with U and vice versa, which has a format or a portion of a format disclosed herein.

In some embodiments, oligonucleotides, e.g., ds oligonucleotides targeting HSD17B13 are stereorandom. In some embodiments, ds oligonucleotides targeting HSD17B13 are chirally controlled. In some embodiments, a ds oligonucleotide targeting HSD17B13 is chirally pure (or “stereopure”, “stereochemically pure”), wherein the oligonucleotide exists as a single stereoisomeric form (in many cases a single diastereoisomeric (or “diastereomeric”) form as multiple chiral centers may exist in an oligonucleotide, e.g., at linkage phosphorus, sugar carbon, etc.). As appreciated by those skilled in the art, a chirally pure oligonucleotide is separated from its other stereoisomeric forms (to the extent that some impurities may exist as chemical and biological processes, selectivities and/or purifications etc. rarely, if ever, go to absolute completeness). In a chirally pure oligonucleotide, each chiral center is independently defined with respect to its configuration (for a chirally pure oligonucleotide, each internucleotidic linkage is independently stereodefined or chirally controlled). In contrast to chirally controlled and chirally pure oligonucleotides which comprise stereodefined linkage phosphorus, racemic (or “stereorandom”, “non-chirally controlled”) oligonucleotides comprising chiral linkage phosphorus, e.g., from traditional phosphoramidite oligonucleotide synthesis without stereochemical control during coupling steps in combination with traditional sulfurization (creating stereorandom phosphorothioate internucleotidic linkages), refer to a random mixture of various stereoisomers (typically diastereoisomers (or “diastereomers”) as there are multiple chiral centers in an oligonucleotide; e.g., from traditional oligonucleotide preparation using reagents containing no chiral elements other than those in nucleosides and linkage phosphorus). For example, for A*A*A wherein * is a phosphorothioate internucleotidic linkage (which comprises a chiral linkage phosphorus), a racemic oligonucleotide preparation includes four diastereomers [2²=4, considering the two chiral linkage phosphorus, each of which can exist in either of two configurations (Sp or Rp)]: A *S A *S A, A *S A *R A, A *R A *S A, and A *R A *R A, wherein *S represents a Sp phosphorothioate internucleotidic linkage and *R represents a Rp phosphorothioate internucleotidic linkage. For a chirally pure oligonucleotide, e.g., A *S A *S A, it exists in a single stereoisomeric form and it is separated from the other stereoisomers (e.g., the diastereomers A *S A *R A, A *R A *S A, and A *R A *R A).

In some embodiments, ds oligonucleotides targeting HSD17B13 comprise 1, 2, 3, 4, 5, 6, 7, 8, 9, 10 or more stereorandom internucleotidic linkages (mixture of Rp and Sp linkage phosphorus at the internucleotidic linkage, e.g., from traditional non-chirally controlled oligonucleotide synthesis). In some embodiments, ds oligonucleotides targeting HSD17B13 comprise one or more (e.g., 1-50, 1-40, 1-30, 1-25, 1-20, 1, 2, 3, 4, 5, 6, 7, 8, 9, 10, 11, 12, 13, 14, 15, 16, 17, 18, 19, 20, 21, 22, 23, 24, 25 or more) chirally controlled internucleotidic linkages (Rp or Sp linkage phosphorus at the internucleotidic linkage, e.g., from chirally controlled oligonucleotide synthesis). In some embodiments, an internucleotidic linkage is a phosphorothioate internucleotidic linkage. In some embodiments, an internucleotidic linkage is a stereorandom phosphorothioate internucleotidic linkage. In some embodiments, an internucleotidic linkage is a chirally controlled phosphorothioate internucleotidic linkage.

Among other things, the present disclosure provides technologies for preparing chirally controlled (in some embodiments, stereochemically pure) oligonucleotides. In some embodiments, oligonucleotides are stereochemically pure. In some embodiments, oligonucleotides of the present disclosure are about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 15%, 20%, 25%, 30%, 35%, 40%, 45%, 50%, 55%, 60%, 65%, 70%, 75%, 80%, 85%, 90%, 95%, or 100%, or at least about 5%, 10%, 15%, 20%, 25%, 30%, 35%, 40%, 45%, 50%, 55%, 60%, 65%, 70%, 75%, 80%, 85%, 90%, 95%, or 99%, pure. In some embodiments, internucleotidic linkages of oligonucleotides comprise or consist of one or more (e.g., 1-50, 1-40, 1-30, 1-25, 1-20, 5-50, 5-40, 5-30, 5-25, 5-20, 1, 2, 3, 4, 5, 6, 7, 8, 9, 10, 11, 12, 13, 14, 15, 16, 17, 18, 19, 20, 21, 22, 23, 24, 25 or more) chiral internucleotidic linkages, each of which independently has a diastereopurity of at least 80%, 85%, 90%, 91%, 92%, 93%, 94%, 95%, 96%, 97%, 98%, 99% or 99.5%, typically at least 90%, 91%, 92%, 93%, 94%, 95%, 96%, 97%, 98%, 99% or 99.5%. In some embodiments, oligonucleotides of the present disclosure, e.g., Ds oligonucleotides targeting HSD17B13, have a diastereopurity of (DS)^{CIL}, wherein DS is a diastereopurity as described in the present disclosure (e.g., 90%, 91%, 92%, 93%, 94%, 95%, 96%, 97%, 98%, 99% or 99.5% or more) and CIL is the number of chirally controlled internucleotidic linkages (e.g., 1-50, 1-40, 1-30, 1-25, 1-20, 5-50, 5-40, 5-30, 5-25, 5-20, 1, 2, 3, 4, 5, 6, 7, 8, 9, 10, 11, 12, 13, 14, 15, 16, 17, 18, 19, 20, 21, 22, 23, 24, 25 or more). In some embodiments, DS is 95%-100%. In some embodiments, each internucleotidic linkage is independently chirally controlled, and CIL is the number of chirally controlled internucleotidic linkages.

As examples, certain ds oligonucleotides targeting HSD17B13 comprising certain example base sequences, nucleobase modifications and patterns thereof, sugar modifications and patterns thereof, internucleotidic linkages and patterns thereof, linkage phosphorus stereochemistry and patterns thereof, linkers, and/or additional chemical moieties are presented in Table 1, below. Among other things, oligonucleotides, e.g., those in Table 1, may be utilized to target a HSD17B13 transcript, e.g., to reduce the level of a HSD17B13 transcript and/or a product thereof.

In certain exemplary embodiments, the ds oligonucleotide targeting HSD17B13 of the present disclosure comprises the base sequence, nucleobase modifications and patterns thereof, sugar modifications and patterns thereof, internucleotidic linkages and patterns thereof, linkage phosphorus stereochemistry and patterns thereof, linkers, and/or additional chemical moieties of: WV-42589; WV-47139; WV-47159; WV-49590; or WV-49591. In certain exemplary embodiments, a ds oligonucleotide targeting HSD17B13 of the present disclosure comprises a guide strand comprising the base sequence, nucleobase modifications and patterns thereof, sugar modifications and patterns thereof, internucleotidic linkages and patterns thereof, linkage phosphorus stereochemistry and patterns thereof, linkers, and/or additional chemical moieties of: WV-47139; WV-47159; WV-49590; or WV-49591. In certain exemplary embodiments, a ds oligonucleotide targeting HSD17B13 of the present disclosure comprises a passenger strand comprising the base sequence, nucleobase modifications and patterns thereof, sugar modifications and patterns thereof, internucleotidic linkages and patterns thereof, linkage phosphorus stereochemistry and patterns thereof, linkers, and/or additional chemical moieties of WV-42589. In certain exemplary embodiments, a ds oligonucleotide targeting HSD17B13 of the present disclosure comprises a guide strand comprising the base sequence, nucleobase modifications and patterns thereof, sugar modifications and patterns thereof, internucleotidic linkages and patterns thereof, linkage phosphorus stereochemistry and patterns thereof, linkers, and/or additional chemical moieties of: WV-47139; WV-47159; WV-49590; or WV-49591 and a passenger strand comprising the base sequence, nucleobase modifications and patterns thereof, sugar modifications and patterns thereof, internucleotidic linkages and patterns thereof, linkage phosphorus stereochemistry and patterns thereof, linkers, and/or additional chemical moieties of WV-42589.

In certain exemplary embodiments, a ds oligonucleotide targeting HSD17B13 of the present disclosure comprises a guide strand comprising the base sequence, nucleobase modifications and patterns thereof, sugar modifications and patterns thereof, internucleotidic linkages and patterns thereof, linkage phosphorus stereochemistry and patterns thereof, linkers, and/or additional chemical moieties of WV-47139 and a passenger strand comprising the base sequence, nucleobase modifications and patterns thereof, sugar modifications and patterns thereof, internucleotidic linkages and patterns thereof, linkage phosphorus stereochemistry and patterns thereof, linkers, and/or additional chemical moieties of WV-42589. In certain exemplary embodiments, a ds oligonucleotide targeting HSD17B13 of the present disclosure comprises a guide strand comprising WV-47139 and a passenger strand comprising WV-42589.

In certain exemplary embodiments, a ds oligonucleotide targeting HSD17B13 of the present disclosure comprises a guide strand comprising the base sequence, nucleobase modifications and patterns thereof, sugar modifications and patterns thereof, internucleotidic linkages and patterns thereof, linkage phosphorus stereochemistry and patterns thereof, linkers, and/or additional chemical moieties of WV-47159 and a passenger strand comprising the base sequence, nucleobase modifications and patterns thereof, sugar modifications and patterns thereof, internucleotidic linkages and patterns thereof, linkage phosphorus stereochemistry and patterns thereof, linkers, and/or additional chemical moieties of: WV-42589. In certain exemplary embodiments, a ds oligonucleotide targeting HSD17B13 of the present disclosure comprises a guide strand comprising WV-47159 and a passenger strand comprising WV-42589.

In certain exemplary embodiments, a ds oligonucleotide targeting HSD17B13 of the present disclosure comprises a guide strand comprising the base sequence, nucleobase modifications and patterns thereof, sugar modifications and patterns thereof, internucleotidic linkages and patterns thereof, linkage phosphorus stereochemistry and patterns thereof, linkers, and/or additional chemical moieties of WV-49590 and a passenger strand comprising the base sequence, nucleobase modifications and patterns thereof, sugar modifications and patterns thereof, internucleotidic linkages and patterns thereof, linkage phosphorus stereochemistry and patterns thereof, linkers, and/or additional chemical moieties of WV-42589. In certain exemplary embodiments, a ds oligonucleotide targeting HSD17B13 of the present disclosure comprises a guide strand comprising WV-49590 and a passenger strand comprising WV-42589.

In certain exemplary embodiments, a ds oligonucleotide targeting HSD17B13 of the present disclosure comprises a guide strand comprising the base sequence, nucleobase modifications and patterns thereof, sugar modifications and patterns thereof, internucleotidic linkages and patterns thereof, linkage phosphorus stereochemistry and patterns thereof, linkers, and/or additional chemical moieties of WV-49591 and a passenger strand comprising the base sequence, nucleobase modifications and patterns thereof, sugar modifications and patterns thereof, internucleotidic linkages and patterns thereof, linkage phosphorus stereochemistry and patterns thereof, linkers, and/or additional chemical moieties of: WV-42589. In certain exemplary embodiments, a ds oligonucleotide targeting HSD17B13 of the present disclosure comprises a guide strand comprising WV-49591 and a passenger strand comprising WV-42589.

TABLE 1
Example Double Stranded Oligonucleotides Targeting HSD17B13.

| | | | Stereochemistry/ |
|---|---|---|---|
| ID | Description | Naked Sequence | linkage |
| WV-38227 | fU*fC*mUfGmGfUmUfCmAfGmUmCmCfUmUfGmU | UCUGGUUCAGUCCUUGUG | XXOOOOOOOOOOOOOOOOXX |
| | fGmU*mU*mU | UUU | |
| WV-38228 | fU*fU*mCfUmGfGmUfUmCfAmGmUmCfCmUfUmG | UUCUGGUUCAGUCCUUGU | XXOOOOOOOOOOOOOOOOXX |
| | fUmG*mU*mU | GUU | |
| WV-38229 | fU*fU*mUfCmCfUmUfCmUfGmGmUmUfCmAfGmU | UUUCCUUCUGGUUCAGUCC | XXOOOOOOOOOOOOOOOOXX |
| | fCmC*mU*mU | UU | |
| WV-38230 | fU*fC*mUfUmCfCmUfUmCfUmGmGmUfUmCfAmGf | UCUUCCUUCUGGUUCAGUC | XXOOOOOOOOOOOOOOOOXX |
| | UmC*mU*mU | UU | |
| WV-38231 | fU*fC*mUfGmUfCmCfUmCfUmUmCmCfUmUfCmUf | UCUGUCCUCUUCCUUCUGG | XXOOOOOOOOOOOOOOOOXX |
| | GmG*mU*mU | UU | |
| WV-38232 | fU*fG*mCfUmCfUmGfUmCfCmUmCmUfUmCfCmUf | UGCUCUGUCCUCUUCCUUC | XXOOOOOOOOOOOOOOOOXX |
| | UmC*mU*mU | UU | |
| WV-38233 | fU*fU*mUfGmCfUmCfUmGfUmCmCmUfCmUfUmCf | UUUGCUCUGUCCUCUUCCU | XXOOOOOOOOOOOOOOOOXX |
| | CmU*mU*mU | UU | |
| WV-38234 | fU*fU*mUfUmGfCmUfCmUfGmUmCmCfUmCfUmU | UUUUGCUCUGUCCUCUUCC | XXOOOOOOOOOOOOOOOOXX |
| | fCmC*mU*mU | UU | |
| WV-38235 | fU*fG*mGfCmUfUmUfGmCfUmCmUmGfUmCfCmU | UGGCUUUGCUCUGUCCUC | XXOOOOOOOOOOOOOOOOXX |
| | fCmU*mU*mU | UUU | |
| WV-38236 | fU*fU*mGfGmCfUmUfUmGfCmUmCmUfGmUfCmC | UUGGCUUUGCUCUGUCCU | XXOOOOOOOOOOOOOOOOXX |
| | fUmC*mU*mU | CUU | |
| WV-38237 | fU*fC*mAfUmGfGmCfUmUfUmGmCmUfCmUfGmU | UCAUGGCUUUGCUCUGUCC | XXOOOOOOOOOOOOOOOOXX |
| | fCmC*mU*mU | UU | |
| WV-38238 | fU*fU*mCfAmUfGmGfCmUfUmUmGmCfUmCfUmG | UUCAUGGCUUUGCUCUGU | XXOOOOOOOOOOOOOOOOXX |
| | fUmC*mU*mU | CUU | |
| WV-38239 | fU*fU*mGfUmUfCmAfUmGfGmCmUmUfUmGfCmU | UUGUUCAUGGCUUUGCUC | XXOOOOOOOOOOOOOOOOXX |
| | fCmU*mU*mU | UUU | |
| WV-38240 | fU*fG*mAfUmGfUmUfCmAfUmGmGmCfUmUfUmG | UGAUGUUCAUGGCUUUGC | XXOOOOOOOOOOOOOOOOXX |
| | fCmU*mU*mU | UUU | |
| WV-38241 | fU*fU*mGfAmUfGmUfUmCfAmUmGmGfCmUfUmU | UUGAUGUUCAUGGCUUUG | XXOOOOOOOOOOOOOOOOXX |
| | fGmC*mU*mU | CUU | |
| WV-38242 | fU*fA+mGfGmAfUmGfAmUfGmUmUmCfAmUfGmG | UAGGAUGAUGUUCAUGGC | XXOOOOOOOOOOOOOOOOXX |
| | fCmU*mU*mU | UUU | |
| WV-38243 | fU*fC*mUfAmGfGmAfUmGfAmUmGmUfUmCfAmU | UCUAGGAUGAUGUUCAUG | XXOOOOOOOOOOOOOOOOXX |
| | fGmG*mU*mU | GUU | |
| WV-38244 | fU*fU*mCfUmAfGmGfAmUfGmAmUmGfUmUfCmA | UUCUAGGAUGAUGUUCAU | XXOOOOOOOOOOOOOOOOXX |
| | fUmG*mU*mU | GUU | |
| WV-38245 | fU*fU*mUfCmUfAmGfGmAfUmGmAmUfGmUfUmC | UUUCUAGGAUGAUGUUCA | XXOOOOOOOOOOOOOOOOXX |
| | fAmU*mU*mU | UUU | |
| WV-38246 | fU*fU*mUfUmCfUmAfGmGfAmUmGmAfUmGfUmU | UUUUCUAGGAUGAUGUUC | XXOOOOOOOOOOOOOOOOXX |
| | fCmA*mU*mU | AUU | |
| WV-38247 | fU*fA*mUfUmUfCmUfAmGfGmAmUmGfAmUfGmU | UAUUUCUAGGAUGAUGUU | XXOOOOOOOOOOOOOOOOXX |
| | fUmC*mU*mU | CUU | |
| WV-38248 | fU*fA*mGfGmAfUmUfUmCfUmAmGmGfAmUfGmA | UAGGAUUUCUAGGAUGAU | XXOOOOOOOOOOOOOOOOXX |
| | fUmG*mU*mU | GUU | |
| WV-38249 | fU*fA*mGfCmAfGmAfAmGfGmAmUmUfUmCfUmAf | UAGCAGAAGGAUUUCUAG | XXOOOOOOOOOOOOOOOOXX |
| | GmG*mU*mU | GUU | |
| WV-38250 | fU*fG*mAfAmGfCmAfGmAfAmGmGmAfUmUfUmCf | UGAAGCAGAAGGAUUUCU | XXOOOOOOOOOOOOOOOOXX |
| | UmA*mU*mU | AUU | |
| WV-38251 | fU*fC*mAfGmAfAmGfCmAfGmAmAmGfGmAfUmUf | UCAGAAGCAGAAGGAUUUC | XXOOOOOOOOOOOOOOOOXX |
| | UmC*mU*mU | UU | |
| WV-38252 | fU*fU*mCfAmGfAmAfGmCfAmGmAmAfGmGfAmUf | UUCAGAAGCAGAAGGAUU | XXOOOOOOOOOOOOOOOOXX |
| | UmU*mU*mU | UUU | |
| WV-38253 | fU*fG*mAfUmCfAmGfAmAfGmCmAmGfAmAfGmGf | UGAUCAGAAGCAGAAGGAU | XXOOOOOOOOOOOOOOOOXX |
| | AmU*mU*mU | UU | |
| WV-38254 | fU*fG*mGfUmGfAmUfCmAfGmAmAmGfCmAfGmAf | UGGUGAUCAGAAGCAGAAG | XXOOOOOOOOOOOOOOOOXX |
| | AmG*mU*mU | UU | |
| WV-38255 | fU*fG*mAfUmGfGmUfGmAfUmCmAmGfAmAfGmC | UGAUGGUGAUCAGAAGCA | XXOOOOOOOOOOOOOOOOXX |
| | fAmG*mU*mU | GUU | |
| WV-38256 | fU*fU*mGfAmUfGmGfUmGfAmUmCmAfGmAfAmG | UUGAUGGUGAUCAGAAGC | XXOOOOOOOOOOOOOOOOXX |
| | fCmA*mU*mU | AUU | |
| WV-38257 | fU*fA*mUfGmAfUmGfGmUfGmAmUmCfAmGfAmA | UAUGAUGGUGAUCAGAAG | XXOOOOOOOOOOOOOOOOXX |
| | fGmC*mU*mU | CUU | |
| WV-38258 | fU*fA*mGfAmUfGmAfUmGfGmUmGmAfUmCfAmG | UAGAUGAUGGUGAUCAGA | XXOOOOOOOOOOOOOOOOXX |
| | fAmA*mU*mU | AUU | |
| WV-38259 | fU*fA*mGfGmAfGmUfAmGfAmUmGmAfUmGfGmU | UAGGAGUAGAUGAUGGUG | XXOOOOOOOOOOOOOOOOXX |
| | fGmA*mU*mU | AUU | |
| WV-38260 | fU*fA*mGfUmAfGmGfAmGfUmAmGmAfUmGfAmU | UAGUAGGAGUAGAUGAUG | XXOOOOOOOOOOOOOOOOXX |
| | fGmG*mU*mU | GUU | |
| WV-38261 | fU*fA*mAfGmUfAmGfGmAfGmUmAmGfAmUfGmA | UAAGUAGGAGUAGAUGAU | XXOOOOOOOOOOOOOOOOXX |
| | fUmG*mU*mU | GUU | |
| WV-38262 | fU*fC*mCfAmAfGmUfAmGfGmAmGmUfAmGfAmUf | UCCAAGUAGGAGUAGAUGA | XXOOOOOOOOOOOOOOOOXX |
| | GmA*mU*mU | UU | |
| WV-38263 | fU*fA*mCfGmAfCmUfCmCfAmAmGmUfAmGfGmAf | UACGACUCCAAGUAGGAGU | XXOOOOOOOOOOOOOOOOXX |
| | GmU*mU*mU | UU | |
| WV-38264 | fU*fC*mCfAmAfCmGfAmCfUmCmCmAfAmGfUmAf | UCCAACGACUCCAAGUAGG | XXOOOOOOOOOOOOOOOOXX |
| | GmG*mU*mU | UU | |
| WV-38265 | fU*fC*mUfGmAfGmGfAmAfUmGmAmAfAmAfAmCf | UCUGAGGAAUGAAAAACUU | XXOOOOOOOOOOOOOOOOXX |
| | UmU*mU*mU | UU | |
| WV-38266 | fU*fU*mUfUmCfUmCfCmUfCmUmGmAfGmGfAmAf | UUUUCUCCUCUGAGGAAU | XXOOOOOOOOOOOOOOOOXX |
| | UmG*mU*mU | GUU | |
| WV-38267 | fU*fC*mAfGmAfUmUfUmUfCmUmCmCfUmCfUmGf | UCAGAUUUUCUCCUCUGAG | XXOOOOOOOOOOOOOOOOXX |
| | AmG*mU*mU | UU | |
| WV-38268 | fU*fU*mAfAmUfGmAfGmAfAmCmAmAfUmCfUmCf | UUAAUGAGAACAAUCUCCC | XXOOOOOOOOOOOOOOOOXX |
| | CmC*mU*mU | UU | |
| WV-38269 | fU*fG*mUfAmAfUmGfAmGfAmAmCmAfAmUfCmUf | UGUAAUGAGAACAAUCUCC | XXOOOOOOOOOOOOOOOOXX |
| | CmC*mU*mU | UU | |
| WV-38270 | fU*fA*mGfUmAfAmUfGmAfGmAmAmCfAmAfUmCf | UAGUAAUGAGAACAAUCUC | XXOOOOOOOOOOOOOOOOXX |
| | UmC*mU*mU | UU | |
| WV-38271 | fU*fC*mCfAmGfUmAfAmUfGmAmGmAfAmCfAmAf | UCCAGUAAUGAGAACAAUC | XXOOOOOOOOOOOOOOOOXX |
| | UmC*mU*mU | UU | |
| WV-38272 | fU*fA*mGfUmCfUmGfCmCfUmGmCmCfUmAfUmUf | UAGUCUGCCUGCCUAUUCC | XXOOOOOOOOOOOOOOOOXX |
| | CmC*mU*mU | UU | |
| WV-38273 | fU*fU*mAfGmUfCmUfGmCfCmUmGmCfCmUfAmUf | UUAGUCUGCCUGCCUAUUC | XXOOOOOOOOOOOOOOOOXX |
| | UmC*mU*mU | UU | |
| WV-38274 | fU*fU*mUfAmAfUmAfUmCfCmCmAmCfAmGfAmAf | UUUAAUAUCCCACAGAACC | XXOOOOOOOOOOOOOOOOXX |
| | CmC*mU*mU | UU | |
| WV-38275 | fU*fC*mAfCmCfCmAfCmUfUmCmUmUfUmCfUmUf | UCACCCACUUCUUUCUUCA | XXOOOOOOOOOOOOOOOOXX |
| | CmA*mU*mU | UU | |
| WV-38276 | fU*fU*mCfAmCfCmCfAmCfUmUmCmUfUmUfCmUf | UUCACCCACUUCUUUCUUC | XXOOOOOOOOOOOOOOOOXX |
| | UmC*mU*mU | UU | |
| WV-38277 | fU*fA*mCfAmUfCmAfCmCfCmAmCmUfUmCfUmUf | UACAUCACCCACUUCUUUC | XXOOOOOOOOOOOOOOOOXX |
| | UmC*mU*mU | UU | |
| WV-38278 | fU*fU*mAfCmAfUmCfAmCfCmCmAmCfUmUfCmUf | UUACAUCACCCACUUCUUU | XXOOOOOOOOOOOOOOOOXX |
| | UmU*mU*mU | UU | |
| WV-38279 | fU*fG*mUfUmAfCmAfUmCfAmCmCmCfAmCfUmUf | UGUUACAUCACCCACUUCU | XXOOOOOOOOOOOOOOOOXX |
| | CmU*mU*mU | UU | |
| WV-38280 | fU*fU*mGfUmUfAmCfAmUfCmAmCmCfCmAfCmUf | UUGUUACAUCACCCACUUC | XXOOOOOOOOOOOOOOOOXX |
| | UmC*mU*mU | UU | |
| WV-38281 | fU*fU*mUfGmUfUmAfCmAfUmCmAmCfCmCfAmCf | UUUGUUACAUCACCCACUU | XXOOOOOOOOOOOOOOOOXX |
| | UmU*mU*mU | UU | |
| WV-38282 | fU*fC*mAfCmCfAmCfGmAfUmUmGmUfUmAfCmAf | UCACCACGAUUGUUACAUC | XXOOOOOOOOOOOOOOOOXX |
| | UmC*mU*mU | UU | |
| WV-38283 | fU*fA*mUfUmCfAmCfCmAfCmGmAmUfUmGfUmUf | UAUUCACCACGAUUGUUAC | XXOOOOOOOOOOOOOOOOXX |
| | AmC*mU*mU | UU | |
| WV-38284 | fU*fU*mAfUmUfCmAfCmCfAmCmGmAfUmUfGmUf | UUAUUCACCACGAUUGUUA | XXOOOOOOOOOOOOOOOOXX |
| | UmA*mU*mU | UU | |
| WV-38285 | fU*fG*mUfCmCfCmAfGmCfAmUmUmAfUmUfCmAf | UGUCCCAGCAUUAUUCACC | XXOOOOOOOOOOOOOOOOXX |
| | CmC*mU*mU | UU | |
| WV-38286 | fU*fA*mCfUmGfUmCfCmCfAmGmCmAfUmUfAmUf | UACUGUCCCAGCAUUAUUC | XXOOOOOOOOOOOOOOOOXX |
| | UmC*mU*mU | UU | |
| WV-38287 | fU*fU*mAfCmUfGmUfCmCfCmAmGmCfAmUfUmAf | UUACUGUCCCAGCAUUAUU | XXOOOOOOOOOOOOOOOOXX |
| | UmU*mU*mU | UU | |
| WV-38288 | fU*fA*mUfAmCfUmGfUmCfCmCmAmGfCmAfUmUf | UAUACUGUCCCAGCAUUAU | XXOOOOOOOOOOOOOOOOXX |
| | AmU*mU*mU | UU | |
| WV-38289 | fU*fC*mGfGmCfUmGfGmAfUmAmUmAfCmUfGmU | UCGGCUGGAUAUACUGUCC | XXOOOOOOOOOOOOOOOOXX |
| | fCmC*mU*mU | UU | |
| WV-38290 | fU*fU*mCfGmGfCmUfGmGfAmUmAmUfAmCfUmG | UUCGGCUGGAUAUACUGU | XXOOOOOOOOOOOOOOOOXX |
| | fUmC*mU*mU | CUU | |
| WV-38291 | fU*fG*mAfUmCfGmGfCmUfGmGmAmUfAmUfAmCf | UGAUCGGCUGGAUAUACU | XXOOOOOOOOOOOOOOOOXX |
| | UmG*mU*mU | GUU | |
| WV-38292 | fU*fA*mGfAmUfCmGfGmCfUmGmGmAfUmAfUmA | UAGAUCGGCUGGAUAUAC | XXOOOOOOOOOOOOOOOOXX |
| | fCmU*mU*mU | UUU | |
| WV-38293 | fU*fU*mCfUmCfUmUfCmAfUmCmCmUfUmGfGmUf | UUCUCUUCAUCCUUGGUGC | XXOOOOOOOOOOOOOOOOXX |
| | GmC*mU*mU | UU | |
| WV-38294 | fU*fA*mUfCmUfCmUfUmCfAmUmCmCfUmUfGmGf | UAUCUCUUCAUCCUUGGU | XXOOOOOOOOOOOOOOOOXX |
| | UmG*mU*mU | GUU | |
| WV-38295 | fU*fA*mAfUmCfUmCfUmUfCmAmUmCfCmUfUmGf | UAAUCUCUUCAUCCUUGGU | XXOOOOOOOOOOOOOOOOXX |
| | GmU*mU*mU | UU | |
| WV-38296 | fU*fG*mGfUmAfAmUfCmUfCmUmUmCfAmUfCmCf | UGGUAAUCUCUUCAUCCUU | XXOOOOOOOOOOOOOOOOXX |
| | UmU*mU*mU | UU | |
| WV-38297 | fU*fU*mGfGmUfAmAfUmCfUmCmUmUfCmAfUmCf | UUGGUAAUCUCUUCAUCCU | XXOOOOOOOOOOOOOOOOXX |
| | CmU*mU*mU | UU | |
| WV-38298 | fU*fG*mGfAmUfGmUfUmGfAmCmCmUfCmAfAmAf | UGGAUGUUGACCUCAAAU | XXOOOOOOOOOOOOOOOOXX |
| | UmG*mU*mU | GUU | |
| WV-38299 | fU*fA*mGfGmAfUmGfUmUfGmAmCmCfUmCfAmAf | UAGGAUGUUGACCUCAAAU | XXOOOOOOOOOOOOOOOOXX |
| | AmU*mU*mU | UU | |
| WV-38300 | fU*fG*mAfUmCfCmAfAmAfAmAmUmGfUmCfCmUf | UGAUCCAAAAAUGUCCUAG | XXOOOOOOOOOOOOOOOOXX |
| | AmG*mU*mU | UU | |
| WV-38301 | fU*fG*mUfGmAfUmCfCmAfAmAmAmAfUmGfUmCf | UGUGAUCCAAAAAUGUCCU | XXOOOOOOOOOOOOOOOOXX |
| | CmU*mU*mU | UU | |
| WV-38302 | fU*fU*mGfUmGfAmUfCmCfAmAmAmAfAmUfGmUf | UUGUGAUCCAAAAAUGUCC | XXOOOOOOOOOOOOOOOOXX |
| | CmC*mU*mU | UU | |
| WV-38303 | fU*fU*mUfGmUfGmAfUmCfCmAmAmAfAmAfUmGf | UUUGUGAUCCAAAAAUGUC | XXOOOOOOOOOOOOOOOOXX |
| | UmC*mU*mU | UU | |
| WV-38304 | fU*fU*mUfUmGfUmGfAmUfCmCmAmAfAmAfAmUf | UUUUGUGAUCCAAAAAUG | XXOOOOOOOOOOOOOOOOXX |
| | GmU*mU*mU | UUU | |
| WV-38305 | fU*fG*mAfAmGfUmGfCmUfUmUmUmGfUmGfAmU | UGAAGUGCUUUUGUGAUC | XXOOOOOOOOOOOOOOOOXX |
| | fCmC*mU*mU | CUU | |
| WV-38306 | fU*fA*mGfAmAfGmUfGmCfUmUmUmUfGmUfGmA | UAGAAGUGCUUUUGUGAU | XXOOOOOOOOOOOOOOOOXX |
| | fUmC*mU*mU | CUU | |
| WV-38307 | fU*fG*mGfAmAfGmAfAmGfUmGmCmUfUmUfUmG | UGGAAGAAGUGCUUUUGU | XXOOOOOOOOOOOOOOOOXX |
| | fUmG*mU*mU | GUU | |
| WV-38308 | fU*fU*mGfGmAfAmGfAmAfGmUmGmCfUmUfUmU | UUGGAAGAAGUGCUUUUG | XXOOOOOOOOOOOOOOOOXX |
| | fGmU*mU*mU | UUU | |
| WV-38309 | fU*fC*mGfAmUfGmGfAmAfGmAmAmGfUmGfCmU | UCGAUGGAAGAAGUGCUU | XXOOOOOOOOOOOOOOOOXX |
| | fUmU*mU*mU | UUU | |
| WV-38310 | fU*fG*mUfGmGfCmCfAmUfGmAmUmUfUmCfUmC | UGUGGCCAUGAUUUCUCU | XXOOOOOOOOOOOOOOOOXX |
| | fUmC*mU*mU | CUU | |
| WV-38311 | fU*fU*mGfCmUfGmGfAmAfCmAmAmUfAmUfGmG | UUGCUGGAACAAUAUGGG | XXOOOOOOOOOOOOOOOOXX |
| | fGmA*mU*mU | AUU | |
| WV-38312 | fU*fU*mUfGmCfUmGfGmAfAmCmAmAfUmAfUmG | UUUGCUGGAACAAUAUGG | XXOOOOOOOOOOOOOOOOXX |
| | fGmG*mU*mU | GUU | |
| WV-38313 | fU*fU*mUfUmGfCmUfGmGfAmAmCmAfAmUfAmU | UUUUGCUGGAACAAUAUG | XXOOOOOOOOOOOOOOOOXX |
| | fGmG*mU*mU | GUU | |
| WV-38314 | fU*fA*mUfUmUfGmCfUmGfGmAmAmCfAmAfUmAf | UAUUUGCUGGAACAAUAU | XXOOOOOOOOOOOOOOOOXX |
| | UmG*mU*mU | GUU | |
| WV-38315 | fU*fA*mAfUmUfUmGfCmUfGmGmAmAfCmAfAmUf | UAAUUUGCUGGAACAAUA | XXOOOOOOOOOOOOOOOOXX |
| | AmU*mU*mU | UUU | |
| WV-38316 | fU*fG*mUfGmAfAmAfGmCfCmAmAmCfAmGfCmGf | UGUGAAAGCCAACAGCGGC | XXOOOOOOOOOOOOOOOOXX |
| | GmC*mU*mU | UU | |
| WV-38317 | fU*fC*mUfGmUfGmAfAmAfGmCmCmAfAmCfAmGf | UCUGUGAAAGCCAACAGCG | XXOOOOOOOOOOOOOOOOXX |
| | CmG*mU*mU | UU | |
| WV-38318 | fU*fC*mAfGmAfCmCfUmCfUmGmUmGfAmAfAmGf | UCAGACCUCUGUGAAAGCC | XXOOOOOOOOOOOOOOOOXX |
| | CmC*mU*mU | UU | |
| WV-38319 | fU*fG*mUfCmAfGmAfCmCfUmCmUmGfUmGfAmAf | UGUCAGACCUCUGUGAAAG | XXOOOOOOOOOOOOOOOOXX |
| | AmG*mU*mU | UU | |
| WV-38320 | fU*fC*mUfGmAfUmGfUmCfAmGmAmCfCmUfCmUf | UCUGAUGUCAGACCUCUGU | XXOOOOOOOOOOOOOOOOXX |
| | GmU*mU*mU | UU | |
| WV-38321 | fU*fU*mCfUmGfAmUfGmUfCmAmGmAfCmCfUmCf | UUCUGAUGUCAGACCUCUG | XXOOOOOOOOOOOOOOOOXX |
| | UmG*mU*mU | UU | |
| WV-38322 | fU*fG*mAfAmGfUmUfCmUfGmAmUmGfUmCfAmG | UGAAGUUCUGAUGUCAGA | XXOOOOOOOOOOOOOOOOXX |
| | fAmC*mU*mU | CUU | |
| WV-38323 | fU*fA*mCfCmAfGmUfUmUfUmUmCmCfCmAfAmGf | UACCAGUUUUUCCCAAGGC | XXOOOOOOOOOOOOOOOOXX |
| | GmC*mU*mU | UU | |
| WV-38324 | fU*fU*mUfGmAfUmAfCmCfAmGmUmUfUmUfUmC | UUUGAUACCAGUUUUUCCC | XXOOOOOOOOOOOOOOOOXX |
| | fCmC*mU*mU | UU | |
| WV-38325 | fU+fU*mUfUmGfAmUfAmCfCmAmGmUfUmUfUmU | UUUUGAUACCAGUUUUUC | XXOOOOOOOOOOOOOOOOXX |
| | fCmC*mU*mU | CUU | |
| WV-38326 | fU*fG*mAfGmGfUmUfUmUfGmAmUmAfCmCfAmG | UGAGGUUUUGAUACCAGU | XXOOOOOOOOOOOOOOOOXX |
| | fUmU*mU*mU | UUU | |
| WV-38327 | fU*fG*mAfCmAfUmGfAmGfGmUmUmUfUmGfAmU | UGACAUGAGGUUUUGAUA | XXOOOOOOOOOOOOOOOOXX |
| | fAmC*mU*mU | CUU | |
| WV-38328 | fU*fU*mGfGmUfGmAfAmCfCmCmAmGfUmAfUmUf | UUGGUGAACCCAGUAUUCA | XXOOOOOOOOOOOOOOOOXX |
| | CmA*mU*mU | UU | |
| WV-38329 | fU*fU*mUfGmGfUmGfAmAfCmCmCmAfGmUfAmUf | UUUGGUGAACCCAGUAUUC | XXOOOOOOOOOOOOOOOOXX |
| | UmC*mU*mU | UU | |
| WV-38330 | fU*fU*mUfUmGfGmUfGmAfAmCmCmCfAmGfUmAf | UUUUGGUGAACCCAGUAU | XXOOOOOOOOOOOOOOOOXX |
| | UmU*mU*mU | UUU | |
| WV-38331 | fU*fU*mUfUmUfGmGfUmGfAmAmCmCfCmAfGmU | UUUUUGGUGAACCCAGUA | XXOOOOOOOOOOOOOOOOXX |
| | fAmU*mU*mU | UUU | |
| WV-38332 | fU*fU*mGfGmAfUmUfUmUfUmGmGmUfGmAfAmC | UUGGAUUUUUGGUGAACC | XXOOOOOOOOOOOOOOOOXX |
| | fCmC*mU*mU | CUU | |
| WV-38333 | fU*fC*mUfUmGfUmGfCmUfUmGmGmAfUmUfUmU | UCUUGUGCUUGGAUUUUU | XXOOOOOOOOOOOOOOOOXX |
| | fUmG*mU*mU | GUU | |
| WV-38334 | fU*fA*mAfUmCfUmUfGmUfGmCmUmUfGmGfAmU | UAAUCUUGUGCUUGGAUU | XXOOOOOOOOOOOOOOOOXX |
| | fUmU*mU*mU | UUU | |
| WV-38335 | fU*fU*mAfAmUfCmUfUmGfUmGmCmUfUmGfGmA | UUAAUCUUGUGCUUGGAU | XXOOOOOOOOOOOOOOOOXX |
| | fUmU*mU*mU | UUU | |
| WV-38336 | fU*fA*mCfAmGfGmCfCmAfUmAmAmUfCmUfUmGf | UACAGGCCAUAAUCUUGUG | XXOOOOOOOOOOOOOOOOXX |
| | UmG*mU*mU | UU | |
| WV-38337 | fU*fU*mAfCmAfGmGfCmCfAmUmAmAfUmCfUmUf | UUACAGGCCAUAAUCUUGU | XXOOOOOOOOOOOOOOOOXX |
| | GmU*mU*mU | UU | |
| WV-38338 | fU*fA*mUfAmCfAmGfGmCfCmAmUmAfAmUfCmUf | UAUACAGGCCAUAAUCUUG | XXOOOOOOOOOOOOOOOOXX |
| | UmG*mU*mU | UU | |
| WV-38339 | fU*fC*mCfAmAfUmAfCmAfGmGmCmCfAmUfAmAf | UCCAAUACAGGCCAUAAUC | XXOOOOOOOOOOOOOOOOXX |
| | UmC*mU*mU | UU | |
| WV-38340 | fU*fC*mUfGmUfCmUfCmCfAmAmUmAfCmAfGmGf | UCUGUCUCCAAUACAGGCC | XXOOOOOOOOOOOOOOOOXX |
| | CmC*mU*mU | UU | |
| WV-38341 | fU*fC*mAfUmCfUmGfUmCfUmCmCmAfAmUfAmCf | UCAUCUGUCUCCAAUACAG | XXOOOOOOOOOOOOOOOOXX |
| | AmG*mU*mU | UU | |
| WV-38342 | fU*fU*mCfAmUfCmUfGmUfCmUmCmCfAmAfUmAf | UUCAUCUGUCUCCAAUACA | XXOOOOOOOOOOOOOOOOXX |
| | CmA*mU*mU | UU | |
| WV-38343 | fU*fA*mCfGmAfCmUfUmCfAmUmCmUfGmUfCmUf | UACGACUUCAUCUGUCUCC | XXOOOOOOOOOOOOOOOOXX |
| | CmC*mU*mU | UU | |
| WV-38344 | fU*fU*mAfCmGfAmCfUmUfCmAmUmCfUmGfUmCf | UUACGACUUCAUCUGUCUC | XXOOOOOOOOOOOOOOOOXX |
| | UmC*mU*mU | UU | |
| WV-38345 | fU*fC*mUfUmAfCmGfAmCfUmUmCmAfUmCfUmGf | UCUUACGACUUCAUCUGUC | XXOOOOOOOOOOOOOOOOXX |
| | UmC*mU*mU | UU | |
| WV-38346 | fU*fU*mCfUmUfAmCfGmAfCmUmUmCfAmUfCmUf | UUCUUACGACUUCAUCUGU | XXOOOOOOOOOOOOOOOOXX |
| | GmU*mU*mU | UU | |
| WV-38347 | fU*fC*mUfUmCfUmUfAmCfGmAmCmUfUmCfAmUf | UCUUCUUACGACUUCAUCU | XXOOOOOOOOOOOOOOOOXX |
| | CmU*mU*mU | UU | |
| WV-38348 | fU*fC*mAfGmAfCmUfUmCfUmUmAmCfGmAfCmUf | UCAGACUUCUUACGACUUC | XXOOOOOOOOOOOOOOOOXX |
| | UmC*mU*mU | UU | |
| WV-38349 | fU*fA*mUfCmAfGmAfCmUfUmCmUmUfAmCfGmAf | UAUCAGACUUCUUACGACU | XXOOOOOOOOOOOOOOOOXX |
| | CmU*mU*mU | UU | |
| WV-38350 | fU*fU*mCfUmAfUmCfAmGfAmCmUmUfCmUfUmAf | UUCUAUCAGACUUCUUACG | XXOOOOOOOOOOOOOOOOXX |
| | CmG*mU*mU | UU | |
| WV-38351 | fU*fC*mCfAmUfCmUfAmUfCmAmGmAfCmUfUmCf | UCCAUCUAUCAGACUUCUU | XXOOOOOOOOOOOOOOOOXX |
| | UmU*mU*mU | UU | |
| WV-38352 | fU*fU*mCfCmAfUmCfUmAfUmCmAmGfAmCfUmUf | UUCCAUCUAUCAGACUUCU | XXOOOOOOOOOOOOOOOOXX |
| | CmU*mU*mU | UU | |
| WV-38353 | fU*fU*mUfCmCfAmUfCmUfAmUmCmAfGmAfCmUf | UUUCCAUCUAUCAGACUUC | XXOOOOOOOOOOOOOOOOXX |
| | UmC*mU*mU | UU | |
| WV-38354 | fU*fA*mUfUmCfCmAfUmCfUmAmUmCfAmGfAmCf | UAUUCCAUCUAUCAGACUU | XXOOOOOOOOOOOOOOOOXX |
| | UmU*mU*mU | UU | |
| WV-38355 | fU*fA*mGfUmAfUmUfCmCfAmUmCmUfAmUfCmAf | UAGUAUUCCAUCUAUCAGA | XXOOOOOOOOOOOOOOOOXX |
| | GmA*mU*mU | UU | |
| WV-38356 | fU*fA*mAfGmUfAmUfUmCfCmAmUmCfUmAfUmCf | UAAGUAUUCCAUCUAUCAG | XXOOOOOOOOOOOOOOOOXX |
| | AmG*mU*mU | UU | |
| WV-38357 | fU*fU*mGfGmUfAmAfGmUfAmUmUmCfCmAfUmC | UUGGUAAGUAUUCCAUCU | XXOOOOOOOOOOOOOOOOXX |
| | fUmA*mU*mU | AUU | |
| WV-38358 | fU*fU*mUfGmGfUmAfAmGfUmAmUmUfCmCfAmU | UUUGGUAAGUAUUCCAUC | XXOOOOOOOOOOOOOOOOXX |
| | fCmU*mU*mU | UUU | |
| WV-38359 | fU*fA*mUfUmGfGmUfAmAfGmUmAmUfUmCfCmA | UAUUGGUAAGUAUUCCAU | XXOOOOOOOOOOOOOOOOXX |
| | fUmC*mU*mU | CUU | |
| WV-38360 | fU*fU*mAfUmUfGmGfUmAfAmGmUmAfUmUfCmC | UUAUUGGUAAGUAUUCCA | XXOOOOOOOOOOOOOOOOXX |
| | fAmU*mU*mU | UUU | |
| WV-38361 | fU*fC*mUfUmAfUmUfGmGfUmAmAmGfUmAfUmU | UCUUAUUGGUAAGUAUUC | XXOOOOOOOOOOOOOOOOXX |
| | fCmC*mU*mU | CUU | |
| WV-38362 | fU*fU*mCfUmUfAmUfUmGfGmUmAmAfGmUfAmU | UUCUUAUUGGUAAGUAUU | XXOOOOOOOOOOOOOOOOXX |
| | fUmC*mU*mU | CUU | |
| WV-38363 | fU*fU*mUfCmUfUmAfUmUfGmGmUmAfAmGfUmA | UUUCUUAUUGGUAAGUAU | XXOOOOOOOOOOOOOOOOXX |
| | fUmU*mU*mU | UUU | |
| WV-38364 | fU*fA*mAfAmUfCmAfUmUfUmUmCmUfUmAfUmU | UAAAUCAUUUUCUUAUUG | XXOOOOOOOOOOOOOOOOXX |
| | fGmG*mU*mU | GUU | |
| WV-38365 | fU*fA*mAfAmAfUmCfAmUfUmUmUmCfUmUfAmU | UAAAAUCAUUUUCUUAUU | XXOOOOOOOOOOOOOOOOXX |
| | fUmG*mU*mU | GUU | |
| WV-38366 | fU*fG*mGfAmAfCmAfAmAfAmAmUmCfAmUfUmUf | UGGAACAAAAAUCAUUUUC | XXOOOOOOOOOOOOOOOOXX |
| | UmC*mU*mU | UU | |
| WV-38367 | fU*fU*mUfCmAfUmUfUmCfAmUmUmUfUmGfAmU | UUUCAUUUCAUUUUGAUU | XXOOOOOOOOOOOOOOOOXX |
| | fUmU*mU*mU | UUU | |
| WV-38368 | fU*fA*mUfCmAfUmUfAmUfCmAmUmGfCmAfUmAf | UAUCAUUAUCAUGCAUACA | XXOOOOOOOOOOOOOOOOXX |
| | CmA*mU*mU | UU | |
| WV-38369 | fU*fG*mCfUmUfUmGfCmAfGmCmAmUfUmGfAmU | UGCUUUGCAGCAUUGAUU | XXOOOOOOOOOOOOOOOOXX |
| | fUmC*mU*mU | CUU | |
| WV-38370 | fU*fA*mGfCmUfUmUfGmCfAmGmCmAfUmUfGmAf | UAGCUUUGCAGCAUUGAU | XXOOOOOOOOOOOOOOOOXX |
| | UmU*mU*mU | UUU | |
| WV-38371 | fU*fG*mAfCmAfGmGfUmAfAmUmUmAfAmUfCmUf | UGACAGGUAAUUAAUCUU | XXOOOOOOOOOOOOOOOOXX |
| | UmG*mU*mU | GUU | |
| WV-38372 | fU*fU*mGfAmGfAmAfAmCfAmGmGmAfAmGfAmCf | UUGAGAAACAGGAAGACAG | XXOOOOOOOOOOOOOOOOXX |
| | AmG*mU*mU | UU | |
| WV-38373 | fU*fU*mCfUmUfGmAfGmAfAmAmCmAfGmGfAmAf | UUCUUGAGAAACAGGAAGA | XXOOOOOOOOOOOOOOOOXX |
| | GmA*mU*mU | UU | |
| WV-38374 | fU*fU*mUfCmUfUmGfAmGfAmAmAmCfAmGfGmAf | UUUCUUGAGAAACAGGAA | XXOOOOOOOOOOOOOOOOXX |
| | AmG*mU*mU | GUU | |
| WV-38375 | fU*fU*mAfUmUfCmUfUmGfAmGmAmAfAmCfAmGf | UUAUUCUUGAGAAACAGG | XXOOOOOOOOOOOOOOOOXX |
| | GmA*mU*mU | AUU | |
| WV-38376 | fU*fA*mUfAmUfUmCfUmUfGmAmGmAfAmAfCmAf | UAUAUUCUUGAGAAACAG | XXOOOOOOOOOOOOOOOOXX |
| | GmG*mU*mU | GUU | |
| WV-38377 | fU*fG*mAfAmAfGmGfAmAfAmAmAmCfAmGfAmCf | UGAAAGGAAAAACAGACCU | XXOOOOOOOOOOOOOOOOXX |
| | CmU*mU*mU | UU | |
| WV-38378 | fU*fU*mGfAmAfAmGfGmAfAmAmAmAfCmAfGmAf | UUGAAAGGAAAAACAGACC | XXOOOOOOOOOOOOOOOOXX |
| | CmC*mU*mU | UU | |
| WV-38379 | fU*fA*mUfGmUfUmUfAmUfGmUmAmAfGmCfAmC | UAUGUUUAUGUAAGCACA | XXOOOOOOOOOOOOOOOOXX |
| | fAmG*mU*mU | GUU | |
| WV-38380 | fU*fA*mAfGmUfAmUfGmUfUmUmAmUfGmUfAmA | UAAGUAUGUUUAUGUAAG | XXOOOOOOOOOOOOOOOOXX |
| | fGmC*mU*mU | CUU | |
| WV-38381 | fU*fU*mAfAmGfUmAfUmGfUmUmUmAfUmGfUmA | UUAAGUAUGUUUAUGUAA | XXOOOOOOOOOOOOOOOOXX |
| | fAmG*mU*mU | GUU | |
| WV-38382 | fU*fA*mAfAmGfAmAfAmAfCmCmUmUfUmUfAmAf | UAAAGAAAACCUUUUAAGU | XXOOOOOOOOOOOOOOOOXX |
| | GmU*mU*mU | UU | |
| WV-38383 | fU*fU*mAfAmAfGmAfAmAfAmCmCmUfUmUfUmAf | UUAAAGAAAACCUUUUAAG | XXOOOOOOOOOOOOOOOOXX |
| | AmG*mU*mU | UU | |
| WV-38384 | fU*fA*mUfCmUfUmAfAmAfGmAmAmAfAmCfCmUf | UAUCUUAAAGAAAACCUUU | XXOOOOOOOOOOOOOOOOXX |
| | UmU*mU*mU | UU | |
| WV-38385 | fU*fA*mUfAmUfCmUfUmAfAmAmGmAfAmAfAmCf | UAUAUCUUAAAGAAAACCU | XXOOOOOOOOOOOOOOOOXX |
| | CmU*mU*mU | UU | |
| WV-38386 | fU*fA*mAfUmAfUmCfUmUfAmAmAmGfAmAfAmAf | UAAUAUCUUAAAGAAAACC | XXOOOOOOOOOOOOOOOOXX |
| | CmC*mU*mU | UU | |
| WV-38387 | fU*fU*mUfGmUfCmCfAmCfCmUmUmUfAmAfAmUf | UUUGUCCACCUUUAAAUGG | XXOOOOOOOOOOOOOOOOXX |
| | GmG*mU*mU | UU | |
| WV-38388 | fU*fU*mUfUmGfUmCfCmAfCmCmUmUfUmAfAmAf | UUUUGUCCACCUUUAAAU | XXOOOOOOOOOOOOOOOOXX |
| | UmG*mU*mU | GUU | |
| WV-38389 | fU*fU*mUfAmCfUmUfUmUfAmGmGmGfAmGfGmU | UUUACUUUUAGGGAGGUA | XXOOOOOOOOOOOOOOOOXX |
| | fAmG*mU*mU | GUU | |
| WV-38390 | fU*fU*mUfGmUfAmUfUmUfAmCmUmUfUmUfAmG | UUUGUAUUUACUUUUAGG | XXOOOOOOOOOOOOOOOOXX |
| | fGmG*mU*mU | GUU | |
| WV-38391 | fU*fC*mUfUmUfGmUfAmUfUmUmAmCfUmUfUmU | UCUUUGUAUUUACUUUUA | XXOOOOOOOOOOOOOOOOXX |
| | fAmG*mU*mU | GUU | |
| WV-38392 | fU*fA*mCfAmGfUmCfUmUfAmAmAmCfCmUfUmCf | UACAGUCUUAAACCUUCCC | XXOOOOOOOOOOOOOOOOXX |
| | CmC*mU*mU | UU | |
| WV-38393 | fU*fG*mAfAmCfAmGfUmCfUmUmAmAfAmCfCmUf | UGAACAGUCUUAAACCUUC | XXOOOOOOOOOOOOOOOOXX |
| | UmC*mU*mU | UU | |
| WV-38394 | fU*fU*mGfAmAfCmAfGmUfCmUmUmAfAmAfCmCf | UUGAACAGUCUUAAACCUU | XXOOOOOOOOOOOOOOOOXX |
| | UmU*mU*mU | UU | |
| WV-38395 | fU*fC*mUfUmGfAmAfCmAfGmUmCmUfUmAfAmAf | UCUUGAACAGUCUUAAACC | XXOOOOOOOOOOOOOOOOXX |
| | CmC*mU*mU | UU | |
| WV-38396 | fU*fA*mCfUmUfGmAfAmCfAmGmUmCfUmUfAmAf | UACUUGAACAGUCUUAAAC | XXOOOOOOOOOOOOOOOOXX |
| | AmC*mU*mU | UU | |
| WV-38397 | fU*fA*mUfGmCfUmAfCmUfUmGmAmAfCmAfGmUf | UAUGCUACUUGAACAGUCU | XXOOOOOOOOOOOOOOOOXX |
| | CmU*mU*mU | UU | |
| WV-38398 | fU*fU*mGfGmAfAmUfGmCfUmAmCmUfUmGfAmA | UUGGAAUGCUACUUGAACA | XXOOOOOOOOOOOOOOOOXX |
| | fCmA*mU*mU | UU | |
| WV-38399 | fU*fU*mUfGmGfAmAfUmGfCmUmAmCfUmUfGmA | UUUGGAAUGCUACUUGAA | XXOOOOOOOOOOOOOOOOXX |
| | fAmC*mU*mU | CUU | |
| WV-38400 | fU*fA*mCfAmGfAmUfUmGfGmAmAmUfGmCfUmAf | UACAGAUUGGAAUGCUACU | XXOOOOOOOOOOOOOOOOXX |
| | CmU*mU*mU | UU | |
| WV-38401 | fU*fC*mUfUmGfUmUfGmAfUmAmUmUfCmUfGmU | UCUUGUUGAUAUUCUGUG | XXOOOOOOOOOOOOOOOOXX |
| | fGmG*mU*mU | GUU | |
| WV-38402 | fU*fU*mCfUmUfGmUfUmGfAmUmAmUfUmCfUmG | UUCUUGUUGAUAUUCUGU | XXOOOOOOOOOOOOOOOOXX |
| | fUmG*mU*mU | GUU | |
| WV-38403 | fU*fG*mUfUmCfUmUfGmUfUmGmAmUfAmUfUmC | UGUUCUUGUUGAUAUUCU | XXOOOOOOOOOOOOOOOOXX |
| | fUmG*mU*mU | GUU | |
| WV-38404 | fU*fG*mUfGmUfUmCfUmUfGmUmUmGfAmUfAm | UGUGUUCUUGUUGAUAUU | XXOOOOOOOOOOOOOOOOXX |
| | UfUmC*mU*mU | CUU | |
| WV-38405 | fU*fC*mAfUmUfCmUfGmUfGmUmUmCfUmUfGmU | UCAUUCUGUGUUCUUGUU | XXOOOOOOOOOOOOOOOOXX |
| | fUmG*mU*mU | GUU | |
| WV-38406 | fU*fA*mGfCmUfGmUfGmCfAmCmUmCfAmUfUmCf | UAGCUGUGCACUCAUUCUG | XXOOOOOOOOOOOOOOOOXX |
| | UmG*mU*mU | UU | |
| WV-38407 | fU*fU*mAfGmCfUmGfUmGfCmAmCmUfCmAfUmUf | UUAGCUGUGCACUCAUUCU | XXOOOOOOOOOOOOOOOOXX |
| | CmU*mU*mU | UU | |
| WV-38408 | fU*fA*mGfGmGfAmAfAmUfCmUmUmUfCmAfAmAf | UAGGGAAAUCUUUCAAAU | XXOOOOOOOOOOOOOOOOXX |
| | UmG*mU*mU | GUU | |
| WV-38409 | fU*fA*mGfAmGfUmUfGmCfAmCmCmGfUmUfUmU | UAGAGUUGCACCGUUUUG | XXOOOOOOOOOOOOOOOOXX |
| | fGmG*mU*mU | GUU | |
| WV-38410 | fU*fC*mCfAmGfAmAfUmAfGmAmGmUfUmGfCmAf | UCCAGAAUAGAGUUGCACC | XXOOOOOOOOOOOOOOOOXX |
| | CmC*mU*mU | UU | |
| WV-38411 | fU*fA*mGfUmCfCmAfGmAfAmUmAmGfAmGfUmUf | UAGUCCAGAAUAGAGUUGC | XXOOOOOOOOOOOOOOOOXX |
| | GmC*mU*mU | UU | |
| WV-38412 | fU*fU*mUfGmAfGmCfCmUfAmAmAmAfUmUfGmU | UUUGAGCCUAAAAUUGUC | XXOOOOOOOOOOOOOOOOXX |
| | fCmU*mU*mU | UUU | |
| WV-38413 | fU*fU*mUfUmGfAmGfCmCfUmAmAmAfAmUfUmG | UUUUGAGCCUAAAAUUGU | XXOOOOOOOOOOOOOOOOXX |
| | fUmC*mU*mU | CUU | |
| WV-38414 | fU*fA*mGfCmUfUmUfAmAfUmUmUmUfUmGfAmG | UAGCUUUAAUUUUUGAGC | XXOOOOOOOOOOOOOOOOXX |
| | fCmC*mU*mU | CUU | |
| WV-38415 | fU*fU*mAfGmCfUmUfUmAfAmUmUmUfUmUfGmA | UUAGCUUUAAUUUUUGAG | XXOOOOOOOOOOOOOOOOXX |
| | fGmC*mU*mU | CUU | |
| WV-38416 | fU*fU*mCfCmUfUmUfUmCfCmUmGmUfGmUfUmA | UUCCUUUUCCUGUGUUAG | XXOOOOOOOOOOOOOOOOXX |
| | fGmC*mU*mU | CUU | |
| WV-38417 | fU*fA*mAfUmAfGmCfCmAfGmUmAmCfAmGfUmUf | UAAUAGCCAGUACAGUUCC | XXOOOOOOOOOOOOOOOOXX |
| | CmC*mU*mU | UU | |
| WV-38418 | fU*fU*mAfGmUfCmUfUmGfAmUmGmUfAmGfUmG | UUAGUCUUGAUGUAGUGG | XXOOOOOOOOOOOOOOOOXX |
| | fGmG*mU*mU | GUU | |
| WV-38419 | mAmCfAmCfAmAfGfGfAmCfUmGfAmAfCmCfAmGf | ACACAAGGACUGAACCAGA | OOOOOOOOOOOOOOOOOO |
| | A | | |
| WV-38420 | mCmAfCmAfAmGfGfAfCmUfGmAfAmCfCmAfGmAf | CACAAGGACUGAACCAGAA | OOOOOOOOOOOOOOOOOO |
| | A | | |
| WV-38421 | mGmGfAmCfUmGfAfAfCmCfAmGfAmAfGmGfAmAf | GGACUGAACCAGAAGGAAA | OOOOOOOOOOOOOOOOOO |
| | A | | |
| WV-38422 | mGmAfCmUfGmAfAfCfCmAfGmAfAmGfGmAfAmGf | GACUGAACCAGAAGGAAGA | OOOOOOOOOOOOOOOOOO |
| | A | | |
| WV-38423 | mCmCfAmGfAmAfGfGfAmAfGmAfGmGfAmCfAmGf | CCAGAAGGAAGAGGACAGA | OOOOOOOOOOOOOOOOOO |
| | A | | |
| WV-38424 | mGmAfAmGfGmAfAfGfAmGfGmAfCmAfGmAfGmCf | GAAGGAAGAGGACAGAGCA | OOOOOOOOOOOOOOOOOO |
| | A | | |
| WV-38425 | mAmGfGmAfAmGfAfGfGmAfCmAfGmAfGmCfAmAf | AGGAAGAGGACAGAGCAAA | OOOOOOOOOOOOOOOOOO |
| | A | | |
| WV-38426 | mGmGfAmAfGmAfGfGfAmCfAmGfAmGfCmAfAmAf | GGAAGAGGACAGAGCAAAA | OOOOOOOOOOOOOOOOOO |
| | A | | |
| WV-38427 | mAmGfAmGfGmAfCfAfGmAfGmCfAmAfAmGfCmCf | AGAGGACAGAGCAAAGCCA | OOOOOOOOOOOOOOOOOO |
| | A | | |
| WV-38428 | mGmAfGmGfAmCfAfGfAmGfCmAfAmAfGmCfCmAf | GAGGACAGAGCAAAGCCAA | OOOOOOOOOOOOOOOOOO |
| | A | | |
| WV-38429 | mGmGfAmCfAmGfAfGfCmAfAmAfGmCfCmAfUmGf | GGACAGAGCAAAGCCAUGA | OOOOOOOOOOOOOOOOOO |
| | A | | |
| WV-38430 | mGmAfCmAfGmAfGfCfAmAfAmGfCmCfAmUfGmAf | GACAGAGCAAAGCCAUGAA | OOOOOOOOOOOOOOOOOO |
| | A | | |
| WV-38431 | mAmGfAmGfCmAfAfAfGmCfCmAfUmGfAmAfCmAf | AGAGCAAAGCCAUGAACAA | OOOOOOOOOOOOOOOOOO |
| | A | | |
| WV-38432 | mAmGfCmAfAmAfGfCfCmAfUmGfAmAfCmAfUmCf | AGCAAAGCCAUGAACAUCA | OOOOOOOOOOOOOOOOOO |
| | A | | |
| WV-38433 | mGmCfAmAfAmGfCfCfAmUfGmAfAmCfAmUfCmAf | GCAAAGCCAUGAACAUCAA | OOOOOOOOOOOOOOOOOO |
| | A | | |
| WV-38434 | mAmGfCmCfAmUfGfAfAmCfAmUfCmAfUmCfCmUf | AGCCAUGAACAUCAUCCUA | OOOOOOOOOOOOOOOOOO |
| | A | | |
| WV-38435 | mCmCfAmUfGmAfAfCfAmUfCmAfUmCfCmUfAmGf | CCAUGAACAUCAUCCUAGA | OOOOOOOOOOOOOOOOOO |
| | A | | |
| WV-38436 | mCmAfUmGfAmAfCfAfUmCfAmUfCmCfUmAfGmAf | CAUGAACAUCAUCCUAGAA | OOOOOOOOOOOOOOOOOO |
| | A | | |
| WV-38437 | mAmUfGmAfAmCfAfUfCmAfUmCfCmUfAmGfAmAf | AUGAACAUCAUCCUAGAAA | OOOOOOOOOOOOOOOOOO |
| | A | | |
| WV-38438 | mUmGfAmAfCmAfUfCfAmUfCmCfUmAfGmAfAmAf | UGAACAUCAUCCUAGAAAA | OOOOOOOOOOOOOOOOOO |
| | A | | |
| WV-38439 | mGmAfAmCfAmUfCfAfUmCfCmUfAmGfAmAfAmUf | GAACAUCAUCCUAGAAAUA | OOOOOOOOOOOOOOOOOO |
| | A | | |
| WV-38440 | mCmAfUmCfAmUfCfCfUmAfGmAfAmAfUmCfCmUf | CAUCAUCCUAGAAAUCCUA | OOOOOOOOOOOOOOOOOO |
| | A | | |
| WV-38441 | mCmCfUmAfGmAfAfAfUmCfCmUfUmCfUmGfCmUf | CCUAGAAAUCCUUCUGCUA | OOOOOOOOOOOOOOOOOO |
| | A | | |
| WV-38442 | mUmAfGmAfAmAfUfCfCmUfUmCfUmGfCmUfUmCf | UAGAAAUCCUUCUGCUUCA | OOOOOOOOOOOOOOOOOO |
| | A | | |
| WV-38443 | mGmAfAmAfUmCfCfUfUmCfUmGfCmUfUmCfUmGf | GAAAUCCUUCUGCUUCUGA | OOOOOOOOOOOOOOOOOO |
| | A | | |
| WV-38444 | mAmAfAmUfCmCfUfUfCmUfGmCfUmUfCmUfGmAf | AAAUCCUUCUGCUUCUGAA | OOOOOOOOOOOOOOOOOO |
| | A | | |
| WV-38445 | mAmUfCmCfUmUfCfUfGmCfUmUfCmUfGmAfUmCf | AUCCUUCUGCUUCUGAUCA | OOOOOOOOOOOOOOOOOO |
| | A | | |
| WV-38446 | mCmUfUmCfUmGfCfUfUmCfUmGfAmUfCmAfCmCf | CUUCUGCUUCUGAUCACCA | OOOOOOOOOOOOOOOOOO |
| | A | | |
| WV-38447 | mCmUfGmCfUmUfCfUfGmAfUmCfAmCfCmAfUmCf | CUGCUUCUGAUCACCAUCA | OOOOOOOOOOOOOOOOOO |
| | A | | |
| WV-38448 | mUmGfCmUfUmCfUfGfAmUfCmAfCmCfAmUfCmAf | UGCUUCUGAUCACCAUCAA | OOOOOOOOOOOOOOOOOO |
| | A | | |
| WV-38449 | mGmCfUmUfCmUfGfAfUmCfAmCfCmAfUmCfAmUf | GCUUCUGAUCACCAUCAUA | OOOOOOOOOOOOOOOOOO |
| | A | | |
| WV-38450 | mUmUfCmUfGmAfUfCfAmCfCmAfUmCfAmUfCmUf | UUCUGAUCACCAUCAUCUA | OOOOOOOOOOOOOOOOOO |
| | A | | |
| WV-38451 | mUmCfAmCfCmAfUfCfAmUfCmUfAmCfUmCfCmUf | UCACCAUCAUCUACUCCUA | OOOOOOOOOOOOOOOOOO |
| | A | | |
| WV-38452 | mCmCfAmUfCmAfUfCfUmAfCmUfCmCfUmAfCmUf | CCAUCAUCUACUCCUACUA | OOOOOOOOOOOOOOOOOO |
| | A | | |
| WV-38453 | mCmAfUmCfAmUfCfUfAmCfUmCfCmUfAmCfUmUf | CAUCAUCUACUCCUACUUA | OOOOOOOOOOOOOOOOOO |
| | A | | |
| WV-38454 | mUmCfAmUfCmUfAfCfUmCfCmUfAmCfUmUfGmGf | UCAUCUACUCCUACUUGGA | OOOOOOOOOOOOOOOOOO |
| | A | | |
| WV-38455 | mAmCfUmCfCmUfAfCfUmUfGmGfAmGfUmCfGmUf | ACUCCUACUUGGAGUCGUA | OOOOOOOOOOOOOOOOOO |
| | A | | |
| WV-38456 | mCmCfUmAfCmUfUfGfGmAfGmUfCmGfUmUfGmGf | CCUACUUGGAGUCGUUGG | OOOOOOOOOOOOOOOOOO |
| | A | A | |
| WV-38457 | mAmAfGmUfUmUfUfUfCmAfUmUfCmCfUmCfAmGf | AAGUUUUUCAUUCCUCAGA | OOOOOOOOOOOOOOOOOO |
| | A | | |
| WV-38458 | mCmAfUmUfCmCfUfCfAmGfAmGfGmAfGmAfAmAf | CAUUCCUCAGAGGAGAAAA | OOOOOOOOOOOOOOOOOO |
| | A | | |
| WV-38459 | mCmUfCmAfGmAfGfGfAmGfAmAfAmAfUmCfUmGf | CUCAGAGGAGAAAAUCUGA | OOOOOOOOOOOOOOOOOO |
| | A | | |
| WV-38460 | mGmGfGmAfGmAfUfUfGmUfUmCfUmCfAmUfUmA | GGGAGAUUGUUCUCAUUA | OOOOOOOOOOOOOOOOOO |
| | fA | A | |
| WV-38461 | mGmGfAmGfAmUfUfGfUmUfCmUfCmAfUmUfAmCf | GGAGAUUGUUCUCAUUAC | OOOOOOOOOOOOOOOOOO |
| | A | A | |
| WV-38462 | mGmAfGmAfUmUfGfUfUmCfUmCfAmUfUmAfCmUf | GAGAUUGUUCUCAUUACU | OOOOOOOOOOOOOOOOOO |
| | A | A | |
| WV-38463 | mGmAfUmUfGmUfUfCfUmCfAmUfUmAfCmUfGmG | GAUUGUUCUCAUUACUGG | OOOOOOOOOOOOOOOOOO |
| | fA | A | |
| WV-38464 | mGmGfAmAfUmAfGfGfCmAfGmGfCmAfGmAfCmUf | GGAAUAGGCAGGCAGACUA | OOOOOOOOOOOOOOOOOO |
| | A | | |
| WV-38465 | mGmAfAmUfAmGfGfCfAmGfGmCfAmGfAmCfUmAf | GAAUAGGCAGGCAGACUAA | OOOOOOOOOOOOOOOOOO |
| | A | | |
| WV-38466 | mGmGfUmUfCmUfGfUfGmGfGmAfUmAfUmUfAmA | GGUUCUGUGGGAUAUUAA | OOOOOOOOOOOOOOOOOO |
| | fA | A | |
| WV-38467 | mUmGfAmAfGmAfAfAfGmAfAmGfUmGfGmGfUmG | UGAAGAAAGAAGUGGGUG | OOOOOOOOOOOOOOOOOO |
| | fA | A | |
| WV-38468 | mGmAfAmGfAmAfAfGfAmAfGmUfGmGfGmUfGmAf | GAAGAAAGAAGUGGGUGA | OOOOOOOOOOOOOOOOOO |
| | A | A | |
| WV-38469 | mGmAfAmAfGmAfAfGfUmGfGmGfUmGfAmUfGmU | GAAAGAAGUGGGUGAUGU | OOOOOOOOOOOOOOOOOO |
| | fA | A | |
| WV-38470 | mAmAfAmGfAmAfGfUfGmGfGmUfGmAfUmGfUmA | AAAGAAGUGGGUGAUGUA | OOOOOOOOOOOOOOOOOO |
| | fA | A | |
| WV-38471 | mAmGfAmAfGmUfGfGfGmUfGmAfUmGfUmAfAmC | AGAAGUGGGUGAUGUAAC | OOOOOOOOOOOOOOOOOO |
| | fA | A | |
| WV-38472 | mGmAfAmGfUmGfGfGfUmGfAmUfGmUfAmAfCmA | GAAGUGGGUGAUGUAACA | OOOOOOOOOOOOOOOOOO |
| | fA | A | |
| WV-38473 | mAmAfGmUfGmGfGfUfGmAfUmGfUmAfAmCfAmAf | AAGUGGGUGAUGUAACAA | OOOOOOOOOOOOOOOOOO |
| | A | A | |
| WV-38474 | mGmAfUmGfUmAfAfCfAmAfUmCfGmUfGmGfUmGf | GAUGUAACAAUCGUGGUG | OOOOOOOOOOOOOOOOOO |
| | A | A | |
| WV-38475 | mGmUfAmAfCmAfAfUfCmGfUmGfGmUfGmAfAmUf | GUAACAAUCGUGGUGAAU | OOOOOOOOOOOOOOOOOO |
| | A | A | |
| WV-38476 | mUmAfAmCfAmAfUfCfGmUfGmGfUmGfAmAfUmAf | UAACAAUCGUGGUGAAUAA | OOOOOOOOOOOOOOOOOO |
| | A | | |
| WV-38477 | mGmGfUmGfAmAfUfAfAmUfGmCfUmGfGmGfAmCf | GGUGAAUAAUGCUGGGAC | OOOOOOOOOOOOOOOOOO |
| | A | A | |
| WV-38478 | mGmAfAmUfAmAfUfGfCmUfGmGfGmAfCmAfGmUf | GAAUAAUGCUGGGACAGUA | OOOOOOOOOOOOOOOOOO |
| | A | | |
| WV-38479 | mAmAfUmAfAmUfGfCfUmGfGmGfAmCfAmGfUmAf | AAUAAUGCUGGGACAGUAA | OOOOOOOOOOOOOOOOOO |
| | A | | |
| WV-38480 | mAmUfAmAfUmGfCfUfGmGfGmAfCmAfGmUfAmUf | AUAAUGCUGGGACAGUAU | OOOOOOOOOOOOOOOOOO |
| | A | A | |
| WV-38481 | mGmGfAmCfAmGfUfAfUmAfUmCfCmAfGmCfCmGf | GGACAGUAUAUCCAGCCGA | OOOOOOOOOOOOOOOOOO |
| | A | | |
| WV-38482 | mGmAfCmAfGmUfAfUfAmUfCmCfAmGfCmCfGmAf | GACAGUAUAUCCAGCCGAA | OOOOOOOOOOOOOOOOOO |
| | A | | |
| WV-38483 | mCmAfGmUfAmUfAfUfCmCfAmGfCmCfGmAfUmCf | CAGUAUAUCCAGCCGAUCA | OOOOOOOOOOOOOOOOOO |
| | A | | |
| WV-38484 | mAmGfUmAfUmAfUfCfCmAfGmCfCmGfAmUfCmUf | AGUAUAUCCAGCCGAUCUA | OOOOOOOOOOOOOOOOOO |
| | A | | |
| WV-38485 | mGmCfAmCfCmAfAfGfGmAfUmGfAmAfGmAfGmAf | GCACCAAGGAUGAAGAGAA | OOOOOOOOOOOOOOOOOO |
| | A | | |
| WV-38486 | mCmAfCmCfAmAfGfGfAmUfGmAfAmGfAmGfAmUf | CACCAAGGAUGAAGAGAUA | OOOOOOOOOOOOOOOOOO |
| | A | | |
| WV-38487 | mAmCfCmAfAmGfGfAfUmGfAmAfGmAfGmAfUmUf | ACCAAGGAUGAAGAGAUUA | OOOOOOOOOOOOOOOOOO |
| | A | | |
| WV-38488 | mAmAfGmGfAmUfGfAfAmGfAmGfAmUfUmAfCmCf | AAGGAUGAAGAGAUUACCA | OOOOOOOOOOOOOOOOOO |
| | A | | |
| WV-38489 | mAmGfGmAfUmGfAfAfGmAfGmAfUmUfAmCfCmAf | AGGAUGAAGAGAUUACCAA | OOOOOOOOOOOOOOOOOO |
| | A | | |
| WV-38490 | mCmAfUmUfUmGfAfGfGmUfCmAfAmCfAmUfCmCf | CAUUUGAGGUCAACAUCCA | OOOOOOOOOOOOOOOOOO |
| | A | | |
| WV-38491 | mAmUfUmUfGmAfGfGfUmCfAmAfCmAfUmCfCmUf | AUUUGAGGUCAACAUCCUA | OOOOOOOOOOOOOOOOOO |
| | A | | |
| WV-38492 | mCmUfAmGfGmAfCfAfUmUfUmUfUmGfGmAfUmCf | CUAGGACAUUUUUGGAUC | OOOOOOOOOOOOOOOOOO |
| | A | A | |
| WV-38493 | mAmGfGmAfCmAfUfUfUmUfUmGfGmAfUmCfAmCf | AGGACAUUUUUGGAUCACA | OOOOOOOOOOOOOOOOOO |
| | A | | |
| WV-38494 | mGmGfAmCfAmUfUfUfUmUfGmGfAmUfCmAfCmAf | GGACAUUUUUGGAUCACAA | OOOOOOOOOOOOOOOOOO |
| | A | | |
| WV-38495 | mGmAfCmAfUmUfUfUfUmGfGmAfUmCfAmCfAmAf | GACAUUUUUGGAUCACAAA | OOOOOOOOOOOOOOOOOO |
| | A | | |
| WV-38496 | mAmCfAmUfUmUfUfUfGmGfAmUfCmAfCmAfAmAf | ACAUUUUUGGAUCACAAAA | OOOOOOOOOOOOOOOOOO |
| | A | | |
| WV-38497 | mGmGfAmUfCmAfCfAfAmAfAmGfCmAfCmUfUmCf | GGAUCACAAAAGCACUUCA | OOOOOOOOOOOOOOOOOO |
| | A | | |
| WV-38498 | mGmAfUmCfAmCfAfAfAmAfGmCfAmCfUmUfCmUf | GAUCACAAAAGCACUUCUA | OOOOOOOOOOOOOOOOOO |
| | A | | |
| WV-38499 | mCmAfCmAfAmAfAfGfCmAfCmUfUmCfUmUfCmCf | CACAAAAGCACUUCUUCCA | OOOOOOOOOOOOOOOOOO |
| | A | | |
| WV-38500 | mAmCfAmAfAmAfGfCfAmCfUmUfCmUfUmCfCmAf | ACAAAAGCACUUCUUCCAA | OOOOOOOOOOOOOOOOOO |
| | A | | |
| WV-38501 | mAmAfAmGfCmAfCfUfUmCfUmUfCmCfAmUfCmGf | AAAGCACUUCUUCCAUCGA | OOOOOOOOOOOOOOOOOO |
| | A | | |
| WV-38502 | mGmAfGmAfGmAfAfAfUmCfAmUfGmGfCmCfAmCf | GAGAGAAAUCAUGGCCACA | OOOOOOOOOOOOOOOOOO |
| | A | | |
| WV-38503 | mUmCfCmCfAmUfAfUfUmGfUmUfCmCfAmGfCmAf | UCCCAUAUUGUUCCAGCAA | OOOOOOOOOOOOOOOOOO |
| | A | | |
| WV-38504 | mCmCfCmAfUmAfUfUfGmUfUmCfCmAfGmCfAmAf | CCCAUAUUGUUCCAGCAAA | OOOOOOOOOOOOOOOOOO |
| | A | | |
| WV-38505 | mCmCfAmUfAmUfUfGfUmUfCmCfAmGfCmAfAmAf | CCAUAUUGUUCCAGCAAAA | OOOOOOOOOOOOOOOOOO |
| | A | | |
| WV-38506 | mCmAfUmAfUmUfGfUfUmCfCmAfGmCfAmAfAmUf | CAUAUUGUUCCAGCAAAUA | OOOOOOOOOOOOOOOOOO |
| | A | | |
| WV-38507 | mAmUfAmUfUmGfUfUfCmCfAmGfCmAfAmAfUmUf | AUAUUGUUCCAGCAAAUUA | OOOOOOOOOOOOOOOOOO |
| | A | | |
| WV-38508 | mGmCfCmGfCmUfGfUfUmGfGmCfUmUfUmCfAmCf | GCCGCUGUUGGCUUUCACA | OOOOOOOOOOOOOOOOOO |
| | A | | |
| WV-38509 | mCmGfCmUfGmUfUfGfGmCfUmUfUmCfAmCfAmGf | CGCUGUUGGCUUUCACAGA | OOOOOOOOOOOOOOOOOO |
| | A | | |
| WV-38510 | mGmGfCmUfUmUfCfAfCmAfGmAfGmGfUmCfUmGf | GGCUUUCACAGAGGUCUGA | OOOOOOOOOOOOOOOOOO |
| | A | | |
| WV-38511 | mCmUfUmUfCmAfCfAfGmAfGmGfUmCfUmGfAmCf | CUUUCACAGAGGUCUGACA | OOOOOOOOOOOOOOOOOO |
| | A | | |
| WV-38512 | mAmCfAmGfAmGfGfUfCmUfGmAfCmAfUmCfAmGf | ACAGAGGUCUGACAUCAGA | OOOOOOOOOOOOOOOOOO |
| | A | | |
| WV-38513 | mCmAfGmAfGmGfUfCfUmGfAmCfAmUfCmAfGmAf | CAGAGGUCUGACAUCAGAA | OOOOOOOOOOOOOOOOOO |
| | A | | |
| WV-38514 | mGmUfCmUfGmAfCfAfUmCfAmGfAmAfCmUfUmCf | GUCUGACAUCAGAACUUCA | OOOOOOOOOOOOOOOOOO |
| | A | | |
| WV-38515 | mGmCfCmUfUmGfGfGfAmAfAmAfAmCfUmGfGmUf | GCCUUGGGAAAAACUGGUA | OOOOOOOOOOOOOOOOOO |
| | A | | |
| WV-38516 | mGmGfGmAfAmAfAfAfCmUfGmGfUmAfUmCfAmAf | GGGAAAAACUGGUAUCAAA | OOOOOOOOOOOOOOOOOO |
| | A | | |
| WV-38517 | mGmGfAmAfAmAfAfCfUmGfGmUfAmUfCmAfAmAf | GGAAAAACUGGUAUCAAAA | OOOOOOOOOOOOOOOOOO |
| | A | | |
| WV-38518 | mAmAfCmUfGmGfUfAfUmCfAmAfAmAfCmCfUmCf | AACUGGUAUCAAAACCUCA | OOOOOOOOOOOOOOOOOO |
| | A | | |
| WV-38519 | mGmUfAmUfCmAfAfAfAmCfCmUfCmAfUmGfUmCf | GUAUCAAAACCUCAUGUCA | OOOOOOOOOOOOOOOOOO |
| | A | | |
| WV-38520 | mUmGfAmAfUmAfCfUfGmGfGmUfUmCfAmCfCmAf | UGAAUACUGGGUUCACCAA | OOOOOOOOOOOOOOOOOO |
| | A | | |
| WV-38521 | mGmAfAmUfAmCfUfGfGmGfUmUfCmAfCmCfAmAf | GAAUACUGGGUUCACCAAA | OOOOOOOOOOOOOOOOOO |
| | A | | |
| WV-38522 | mAmAfUmAfCmUfGfGfGmUfUmCfAmCfCmAfAmAf | AAUACUGGGUUCACCAAAA | OOOOOOOOOOOOOOOOOO |
| | A | | |
| WV-38523 | mAmUfAmCfUmGfGfGfUmUfCmAfCmCfAmAfAmAf | AUACUGGGUUCACCAAAAA | OOOOOOOOOOOOOOOOOO |
| | A | | |
| WV-38524 | mGmGfGmUfUmCfAfCfCmAfAmAfAmAfUmCfCmAf | GGGUUCACCAAAAAUCCAA | OOOOOOOOOOOOOOOOOO |
| | A | | |
| WV-38525 | mCmAfAmAfAmAfUfCfCmAfAmGfCmAfCmAfAmGf | CAAAAAUCCAAGCACAAGA | OOOOOOOOOOOOOOOOOO |
| | A | | |
| WV-38526 | mAmAfAmUfCmCfAfAfGmCfAmCfAmAfGmAfUmUf | AAAUCCAAGCACAAGAUUA | OOOOOOOOOOOOOOOOOO |
| | A | | |
| WV-38527 | mAmAfUmCfCmAfAfGfCmAfCmAfAmGfAmUfUmAf | AAUCCAAGCACAAGAUUAA | OOOOOOOOOOOOOOOOOO |
| | A | | |
| WV-38528 | mCmAfCmAfAmGfAfUfUmAfUmGfGmCfCmUfGmUf | CACAAGAUUAUGGCCUGUA | OOOOOOOOOOOOOOOOOO |
| | A | | |
| WV-38529 | mAmCfAmAfGmAfUfUfAmUfGmGfCmCfUmGfUmAf | ACAAGAUUAUGGCCUGUAA | OOOOOOOOOOOOOOOOOO |
| | A | | |
| WV-38530 | mCmAfAmGfAmUfUfAfUmGfGmCfCmUfGmUfAmUf | CAAGAUUAUGGCCUGUAUA | OOOOOOOOOOOOOOOOOO |
| | A | | |
| WV-38531 | mGmAfUmUfAmUfGfGfCmCfUmGfUmAfUmUfGmG | GAUUAUGGCCUGUAUUGG | OOOOOOOOOOOOOOOOOO |
| | fA | A | |
| WV-38532 | mGmGfCmCfUmGfUfAfUmUfGmGfAmGfAmCfAmGf | GGCCUGUAUUGGAGACAG | OOOOOOOOOOOOOOOOOO |
| | A | A | |
| WV-38533 | mCmUfGmUfAmUfUfGfGmAfGmAfCmAfGmAfUmGf | CUGUAUUGGAGACAGAUG | OOOOOOOOOOOOOOOOOO |
| | A | A | |
| WV-38534 | mUmGfUmAfUmUfGfGfAmGfAmCfAmGfAmUfGmA | UGUAUUGGAGACAGAUGA | OOOOOOOOOOOOOOOOOO |
| | fA | A | |
| WV-38535 | mGmGfAmGfAmCfAfGfAmUfGmAfAmGfUmCfGmUf | GGAGACAGAUGAAGUCGUA | OOOOOOOOOOOOOOOOOO |
| | A | | |
| WV-38536 | mGmAfGmAfCmAfGfAfUmGfAmAfGmUfCmGfUmAf | GAGACAGAUGAAGUCGUAA | OOOOOOOOOOOOOOOOOO |
| | A | | |
| WV-38537 | mGmAfCmAfGmAfUfGfAmAfGmUfCmGfUmAfAmGf | GACAGAUGAAGUCGUAAGA | OOOOOOOOOOOOOOOOOO |
| | A | | |
| WV-38538 | mAmCfAmGfAmUfGfAfAmGfUmCfGmUfAmAfGmAf | ACAGAUGAAGUCGUAAGAA | OOOOOOOOOOOOOOOOOO |
| | A | | |
| WV-38539 | mAmGfAmUfGmAfAfGfUmCfGmUfAmAfGmAfAmGf | AGAUGAAGUCGUAAGAAGA | OOOOOOOOOOOOOOOOOO |
| | A | | |
| WV-38540 | mGmAfAmGfUmCfGfUfAmAfGmAfAmGfUmCfUmGf | GAAGUCGUAAGAAGUCUGA | OOOOOOOOOOOOOOOOOO |
| | A | | |
| WV-38541 | mAmGfUmCfGmUfAfAfGmAfAmGfUmCfUmGfAmUf | AGUCGUAAGAAGUCUGAU | OOOOOOOOOOOOOOOOOO |
| | A | A | |
| WV-38542 | mCmGfUmAfAmGfAfAfGmUfCmUfGmAfUmAfGmAf | CGUAAGAAGUCUGAUAGAA | OOOOOOOOOOOOOOOOOO |
| | A | | |
| WV-38543 | mAmAfGmAfAmGfUfCfUmGfAmUfAmGfAmUfGmGf | AAGAAGUCUGAUAGAUGG | OOOOOOOOOOOOOOOOOO |
| | A | A | |
| WV-38544 | mAmGfAmAfGmUfCfUfGmAfUmAfGmAfUmGfGmAf | AGAAGUCUGAUAGAUGGA | OOOOOOOOOOOOOOOOOO |
| | A | A | |
| WV-38545 | mGmAfAmGfUmCfUfGfAmUfAmGfAmUfGmGfAmAf | GAAGUCUGAUAGAUGGAA | OOOOOOOOOOOOOOOOOO |
| | A | A | |
| WV-38546 | mAmAfGmUfCmUfGfAfUmAfGmAfUmGfGmAfAmUf | AAGUCUGAUAGAUGGAAU | OOOOOOOOOOOOOOOOOO |
| | A | A | |
| WV-38547 | mUmCfUmGfAmUfAfGfAmUfGmGfAmAfUmAfCmUf | UCUGAUAGAUGGAAUACU | OOOOOOOOOOOOOOOOOO |
| | A | A | |
| WV-38548 | mCmUfGmAfUmAfGfAfUmGfGmAfAmUfAmCfUmUf | CUGAUAGAUGGAAUACUU | OOOOOOOOOOOOOOOOOO |
| | A | A | |
| WV-38549 | mUmAfGmAfUmGfGfAfAmUfAmCfUmUfAmCfCmAf | UAGAUGGAAUACUUACCAA | OOOOOOOOOOOOOOOOOO |
| | A | | |
| WV-38550 | mAmGfAmUfGmGfAfAfUmAfCmUfUmAfCmCfAmAf | AGAUGGAAUACUUACCAAA | OOOOOOOOOOOOOOOOOO |
| | A | | |
| WV-38551 | mGmAfUmGfGmAfAfUfAmCfUmUfAmCfCmAfAmUf | GAUGGAAUACUUACCAAUA | OOOOOOOOOOOOOOOOOO |
| | A | | |
| WV-38552 | mAmUfGmGfAmAfUfAfCmUfUmAfCmCfAmAfUmAf | AUGGAAUACUUACCAAUAA | OOOOOOOOOOOOOOOOOO |
| | A | | |
| WV-38553 | mGmGfAmAfUmAfCfUfUmAfCmCfAmAfUmAfAmGf | GGAAUACUUACCAAUAAGA | OOOOOOOOOOOOOOOOOO |
| | A | | |
| WV-38554 | mGmAfAmUfAmCfUfUfAmCfCmAfAmUfAmAfGmAf | GAAUACUUACCAAUAAGAA | OOOOOOOOOOOOOOOOOO |
| | A | | |
| WV-38555 | mAmAfUmAfCmUfUfAfCmCfAmAfUmAfAmGfAmAf | AAUACUUACCAAUAAGAAA | OOOOOOOOOOOOOOOOOO |
| | A | | |
| WV-38556 | mCmCfAmAfUmAfAfGfAmAfAmAfUmGfAmUfUmUf | CCAAUAAGAAAAUGAUUUA | OOOOOOOOOOOOOOOOOO |
| | A | | |
| WV-38557 | mCmAfAmUfAmAfGfAfAmAfAmUfGmAfUmUfUmUf | CAAUAAGAAAAUGAUUUUA | OOOOOOOOOOOOOOOOOO |
| | A | | |
| WV-38558 | mGmAfAmAfAmUfGfAfUmUfUmUfUmGfUmUfCmC | GAAAAUGAUUUUUGUUCC | OOOOOOOOOOOOOOOOOO |
| | fA | A | |
| WV-38559 | mAmAfAmUfCmAfAfAfAmUfGmAfAmAfUmGfAmAf | AAAUCAAAAUGAAAUGAAA | OOOOOOOOOOOOOOOOOO |
| | A | | |
| WV-38560 | mUmGfUmAfUmGfCfAfUmGfAmUfAmAfUmGfAmU | UGUAUGCAUGAUAAUGAU | OOOOOOOOOOOOOOOOOO |
| | fA | A | |
| WV-38561 | mGmAfAmUfCmAfAfUfGmCfUmGfCmAfAmAfGmCf | GAAUCAAUGCUGCAAAGCA | OOOOOOOOOOOOOOOOOO |
| | A | | |
| WV-38562 | mAmAfUmCfAmAfUfGfCmUfGmCfAmAfAmGfCmUf | AAUCAAUGCUGCAAAGCUA | OOOOOOOOOOOOOOOOOO |
| | A | | |
| WV-38563 | mCmAfAmGfAmUfUfAfAmUfUmAfCmCfUmGfUmCf | CAAGAUUAAUUACCUGUCA | OOOOOOOOOOOOOOOOOO |
| | A | | |
| WV-38564 | mCmUfGmUfCmUfUfCfCmUfGmUfUmUfCmUfCmAf | CUGUCUUCCUGUUUCUCAA | OOOOOOOOOOOOOOOOOO |
| | A | | |
| WV-38565 | mUmCfUmUfCmCfUfGfUmUfUmCfUmCfAmAfGmAf | UCUUCCUGUUUCUCAAGAA | OOOOOOOOOOOOOOOOOO |
| | A | | |
| WV-38566 | mCmUfUmCfCmUfGfUfUmUfCmUfCmAfAmGfAmAf | CUUCCUGUUUCUCAAGAAA | OOOOOOOOOOOOOOOOOO |
| | A | | |
| WV-38567 | mUmCfCmUfGmUfUfUfCmUfCmAfAmGfAmAfUmAf | UCCUGUUUCUCAAGAAUAA | OOOOOOOOOOOOOOOOOO |
| | A | | |
| WV-38568 | mCmCfUmGfUmUfUfCfUmCfAmAfGmAfAmUfAmUf | CCUGUUUCUCAAGAAUAUA | OOOOOOOOOOOOOOOOOO |
| | A | | |
| WV-38569 | mAmGfGmUfCmUfGfUfUmUfUmUfCmCfUmUfUmC | AGGUCUGUUUUUCCUUUC | OOOOOOOOOOOOOOOOOO |
| | fA | A | |
| WV-38570 | mGmGfUmCfUmGfUfUfUmUfUmCfCmUfUmUfCmA | GGUCUGUUUUUCCUUUCA | OOOOOOOOOOOOOOOOOO |
| | fA | A | |
| WV-38571 | mCmUfGmUfGmCfUfUfAmCfAmUfAmAfAmCfAmUf | CUGUGCUUACAUAAACAUA | OOOOOOOOOOOOOOOOOO |
| | A | | |
| WV-38572 | mGmCfUmUfAmCfAfUfAmAfAmCfAmUfAmCfUmUf | GCUUACAUAAACAUACUUA | OOOOOOOOOOOOOOOOOO |
| | A | | |
| WV-38573 | mCmUfUmAfCmAfUfAfAmAfCmAfUmAfCmUfUmAf | CUUACAUAAACAUACUUAA | OOOOOOOOOOOOOOOOOO |
| | A | | |
| WV-38574 | mAmCfUmUfAmAfAfAfGmGfUmUfUmUfCmUfUmU | ACUUAAAAGGUUUUCUUU | OOOOOOOOOOOOOOOOOO |
| | fA | A | |
| WV-38575 | mCmUfUmAfAmAfAfGfGmUfUmUfUmCfUmUfUmA | CUUAAAAGGUUUUCUUUA | OOOOOOOOOOOOOOOOOO |
| | fA | A | |
| WV-38576 | mAmAfAmGfGmUfUfUfUmCfUmUfUmAfAmGfAmU | AAAGGUUUUCUUUAAGAU | OOOOOOOOOOOOOOOOOO |
| | fA | A | |
| WV-38577 | mAmGfGmUfUmUfUfCfUmUfUmAfAmGfAmUfAmU | AGGUUUUCUUUAAGAUAU | OOOOOOOOOOOOOOOOOO |
| | fA | A | |
| WV-38578 | mGmGfUmUfUmUfCfUfUmUfAmAfGmAfUmAfUmU | GGUUUUCUUUAAGAUAUU | OOOOOOOOOOOOOOOOOO |
| | fA | A | |
| WV-38579 | mCmCfAmUfUmUfAfAfAmGfGmUfGmGfAmCfAmAf | CCAUUUAAAGGUGGACAAA | OOOOOOOOOOOOOOOOOO |
| | A | | |
| WV-38580 | mCmAfUmUfUmAfAfAfGmGfUmGfGmAfCmAfAmAf | CAUUUAAAGGUGGACAAAA | OOOOOOOOOOOOOOOOOO |
| | A | | |
| WV-38581 | mCmUfAmCfCmUfCfCfCmUfAmAfAmAfGmUfAmAf | CUACCUCCCUAAAAGUAAA | OOOOOOOOOOOOOOOOOO |
| | A | | |
| WV-38582 | mCmCfCmUfAmAfAfAfGmUfAmAfAmUfAmCfAmAf | CCCUAAAAGUAAAUACAAA | OOOOOOOOOOOOOOOOOO |
| | A | | |
| WV-38583 | mCmUfAmAfAmAfGfUfAmAfAmUfAmCfAmAfAmGf | CUAAAAGUAAAUACAAAGA | OOOOOOOOOOOOOOOOOO |
| | A | | |
| WV-38584 | mGmGfGmAfAmGfGfUfUmUfAmAfGmAfCmUfGmU | GGGAAGGUUUAAGACUGU | OOOOOOOOOOOOOOOOOO |
| | fA | A | |
| WV-38585 | mGmAfAmGfGmUfUfUfAmAfGmAfCmUfGmUfUmCf | GAAGGUUUAAGACUGUUC | OOOOOOOOOOOOOOOOOO |
| | A | A | |
| WV-38586 | mAmAfGmGfUmUfUfAfAmGfAmCfUmGfUmUfCmAf | AAGGUUUAAGACUGUUCA | OOOOOOOOOOOOOOOOOO |
| | A | A | |
| WV-38587 | mGmGfUmUfUmAfAfGfAmCfUmGfUmUfCmAfAmGf | GGUUUAAGACUGUUCAAG | OOOOOOOOOOOOOOOOOO |
| | A | A | |
| WV-38588 | mGmUfUmUfAmAfGfAfCmUfGmUfUmCfAmAfGmUf | GUUUAAGACUGUUCAAGU | OOOOOOOOOOOOOOOOOO |
| | A | A | |
| WV-38589 | mAmGfAmCfUmGfUfUfCmAfAmGfUmAfGmCfAmUf | AGACUGUUCAAGUAGCAUA | OOOOOOOOOOOOOOOOOO |
| | A | | |
| WV-38590 | mUmGfUmUfCmAfAfGfUmAfGmCfAmUfUmCfCmAf | UGUUCAAGUAGCAUUCCAA | OOOOOOOOOOOOOOOOOO |
| | A | | |
| WV-38591 | mGmUfUmCfAmAfGfUfAmGfCmAfUmUfCmCfAmAf | GUUCAAGUAGCAUUCCAAA | OOOOOOOOOOOOOOOOOO |
| | A | | |
| WV-38592 | mAmGfUmAfGmCfAfUfUmCfCmAfAmUfCmUfGmUf | AGUAGCAUUCCAAUCUGUA | OOOOOOOOOOOOOOOOOO |
| | A | | |
| WV-38593 | mCmCfAmCfAmGfAfAfUmAfUmCfAmAfCmAfAmGf | CCACAGAAUAUCAACAAGA | OOOOOOOOOOOOOOOOOO |
| | A | | |
| WV-38594 | mCmAfCmAfGmAfAfUfAmUfCmAfAmCfAmAfGmAf | CACAGAAUAUCAACAAGAA | OOOOOOOOOOOOOOOOOO |
| | A | | |
| WV-38595 | mCmAfGmAfAmUfAfUfCmAfAmCfAmAfGmAfAmCf | CAGAAUAUCAACAAGAACA | OOOOOOOOOOOOOOOOOO |
| | A | | |
| WV-38596 | mGmAfAmUfAmUfCfAfAmCfAmAfGmAfAmCfAmCf | GAAUAUCAACAAGAACACA | OOOOOOOOOOOOOOOOOO |
| | A | | |
| WV-38597 | mCmAfAmCfAmAfGfAfAmCfAmCfAmGfAmAfUmGf | CAACAAGAACACAGAAUGA | OOOOOOOOOOOOOOOOOO |
| | A | | |
| WV-38598 | mCmAfGmAfAmUfGfAfGmUfGmCfAmCfAmGfCmUf | CAGAAUGAGUGCACAGCUA | OOOOOOOOOOOOOOOOOO |
| | A | | |
| WV-38599 | mAmGfAmAfUmGfAfGfUmGfCmAfCmAfGmCfUmAf | AGAAUGAGUGCACAGCUAA | OOOOOOOOOOOOOOOOOO |
| | A | | |
| WV-38600 | mCmAfUmUfUmGfAfAfAmGfAmUfUmUfCmCfCmUf | CAUUUGAAAGAUUUCCCUA | OOOOOOOOOOOOOOOOOO |
| | A | | |
| WV-38601 | mCmCfAmAfAmAfCfGfGmUfGmCfAmAfCmUfCmUf | CCAAAACGGUGCAACUCUA | OOOOOOOOOOOOOOOOOO |
| | A | | |
| WV-38602 | mGmGfUmGfCmAfAfCfUmCfUmAfUmUfCmUfGmGf | GGUGCAACUCUAUUCUGGA | OOOOOOOOOOOOOOOOOO |
| | A | | |
| WV-38603 | mGmCfAmAfCmUfCfUfAmUfUmCfUmGfGmAfCmUf | GCAACUCUAUUCUGGACUA | OOOOOOOOOOOOOOOOOO |
| | A | | |
| WV-38604 | mAmGfAmCfAmAfUfUfUmUfAmGfGmCfUmCfAmAf | AGACAAUUUUAGGCUCAAA | OOOOOOOOOOOOOOOOOO |
| | A | | |
| WV-38605 | mGmAfCmAfAmUfUfUfUmAfGmGfCmUfCmAfAmAf | GACAAUUUUAGGCUCAAAA | OOOOOOOOOOOOOOOOOO |
| | A | | |
| WV-38606 | mGmGfCmUfCmAfAfAfAmAfUmUfAmAfAmGfCmUf | GGCUCAAAAAUUAAAGCUA | OOOOOOOOOOOOOOOOOO |
| | A | | |
| WV-38607 | mGmCfUmCfAmAfAfAfAmUfUmAfAmAfGmCfUmAf | GCUCAAAAAUUAAAGCUAA | OOOOOOOOOOOOOOOOOO |
| | A | | |
| WV-38608 | mGmCfUmAfAmCfAfCfAmGfGmAfAmAfAmGfGmAf | GCUAACACAGGAAAAGGAA | OOOOOOOOOOOOOOOOOO |
| | A | | |
| WV-38609 | mGmGfAmAfCmUfGfUfAmCfUmGfGmCfUmAfUmUf | GGAACUGUACUGGCUAUU | OOOOOOOOOOOOOOOOOO |
| | A | A | |
| WV-38610 | mCmCfCmAfCmUfAfCfAmUfCmAfAmGfAmCfUmAf | CCCACUACAUCAAGACUAA | OOOOOOOOOOOOOOOOOO |
| | A | | |
| WV-41031 | Mod001L001mG*mGfCmUfUmUfCfAfCmAfGmAfG | GGCUUUCACAGAGGUCUGA | OXOOOOOOOOOOOOOOOOX |
| | mGfUmCfUmG*fA | | |
| WV-41032 | Mod001L001mC*mGfCmUfGmUfUfGfGmCfUmUfU | CGCUGUUGGCUUUCACAGA | OXOOOOOOOOOOOOOOOOX |
| | mCfAmCfAmG*fA | | |
| WV-41033 | Mod001L001mC*mUfUmUfCmAfCfAfGmAfGmGfU | CUUUCACAGAGGUCUGACA | OXOOOOOOOOOOOOOOOOX |
| | mCfUmGfAmC*fA | | |
| WV-41034 | Mod001L001mG*mGfGmAfAmAfAfAfCmUfGmGfU | GGGAAAAACUGGUAUCAAA | OXOOOOOOOOOOOOOOOOX |
| | mAfUmCfAmA*fA | | |
| WV-41035 | Mod001L001mA*mAfGmUfCmUfGfAfUmAfGmAfU | AAGUCUGAUAGAUGGAAU | OXOOOOOOOOOOOOOOOOX |
| | mGfGmAfAmU*fA | A | |
| WV-41036 | Mod001L001mG*mGfAmAfAmAfAfCfUmGfGmUfA | GGAAAAACUGGUAUCAAAA | OXOOOOOOOOOOOOOOOOX |
| | mUfCmAfAmA*fA | | |
| WV-41037 | Mod001L001mC*mAfAmGfAmUfUfAfUmGfGmCfC | CAAGAUUAUGGCCUGUAUA | OXOOOOOOOOOOOOOOOOX |
| | mUfGmUfAmU*fA | | |
| WV-41038 | Mod001L001mG*mGfAmUfCmAfCfAfAmAfAmGfCm | GGAUCACAAAAGCACUUCA | OXOOOOOOOOOOOOOOOOX |
| | AfCmUfUmC*fA | | |
| WV-41039 | Mod001L001mC*mAfAmUfAmAfGfAfAmAfAmUfG | CAAUAAGAAAAUGAUUUUA | OXOOOOOOOOOOOOOOOOX |
| | mAfUmUfUmU*fA | | |
| WV-41040 | Mod001L001mC*mCfAmUfAmUfUfGfUmUfCmCfA | CCAUAUUGUUCCAGCAAAA | OXOOOOOOOOOOOOOOOOX |
| | mGfCmAfAmA*fA | | |
| WV-41041 | Mod001L001mU*mAfGmAfUmGfGfAfAmUfAmCfU | UAGAUGGAAUACUUACCAA | OXOOOOOOOOOOOOOOOOX |
| | mUfAmCfCmA*fA | | |
| WV-41042 | Mod001L001mA*mCfAmAfGmAfUfUfAmUfGmGfC | ACAAGAUUAUGGCCUGUAA | OXOOOOOOOOOOOOOOOOX |
| | mCfUmGfUmA*fA | | |
| WV-41043 | Mod001L001mC*mCfCmAfUmAfUfUfGmUfUmCfC | CCCAUAUUGUUCCAGCAAA | OXOOOOOOOOOOOOOOOOX |
| | mAfGmCfAmA*fA | | |
| WV-41044 | Mod001L001mA*mCfAmGfAmUfGfAfAmGfUmCfG | ACAGAUGAAGUCGUAAGAA | OXOOOOOOOOOOOOOOOOX |
| | mUfAmAfGmA*fA | | |
| WV-41045 | Mod001L001mU*mGfUmAfUmUfGfGfAmGfAmCfA | UGUAUUGGAGACAGAUGA | OXOOOOOOOOOOOOOOOOX |
| | mGfAmUfGmA*fA | A | |
| WV-41046 | Mod001L001mC*mAfCmAfAmGfAfUfUmAfUmGfG | CACAAGAUUAUGGCCUGUA | OXOOOOOOOOOOOOOOOOX |
| | mCfCmUfGmU*fA | | |
| WV-41047 | Mod001L001mG*mAfAmGfUmCfGfUfAmAfGmAfA | GAAGUCGUAAGAAGUCUGA | OXOOOOOOOOOOOOOOOOX |
| | mGfUmCfUmG*fA | | |
| WV-41048 | Mod001L001mG*mUfAmUfCmAfAfAfAmCfCmUfCm | GUAUCAAAACCUCAUGUCA | OXOOOOOOOOOOOOOOOOX |
| | AfUmGfUmC*fA | | |
| WV-41049 | Mod001L001mG*mAfAmUfAmCfUfUfAmCfCmAfAm | GAAUACUUACCAAUAAGAA | OXOOOOOOOOOOOOOOOOX |
| | UfAmAfGmA*fA | | |
| WV-41050 | Mod001L001mA*mGfAmAfGmUfCfUfGmAfUmAfG | AGAAGUCUGAUAGAUGGA | OXOOOOOOOOOOOOOOOOX |
| | mAfUmGfGmA*fA | A | |
| WV-41051 | Mod001L001mA*mUfGmGfAmAfUfAfCmUfUmAfC | AUGGAAUACUUACCAAUAA | OXOOOOOOOOOOOOOOOOX |
| | mCfAmAfUmA*fA | | |
| WV-41052 | Mod001L001mG*mGfCmCfUmGfUfAfUmUfGmGfA | GGCCUGUAUUGGAGACAG | OXOOOOOOOOOOOOOOOOX |
| | mGfAmCfAmG*fA | A | |
| WV-41053 | Mod001L001mG*mGfGmUfUmCfAfCfCmAfAmAfA | GGGUUCACCAAAAAUCCAA | OXOOOOOOOOOOOOOOOOX |
| | mAfUmCfCmA*fA | | |
| WV-41054 | Mod001L001mG*mAfCmAfGmAfUfGfAmAfGmUfC | GACAGAUGAAGUCGUAAGA | OXOOOOOOOOOOOOOOOOX |
| | mGfUmAfAmG*fA | | |
| WV-41055 | Mod001L001mC*mUfGmAfUmAfGfAfUmGfGmAfA | CUGAUAGAUGGAAUACUU | OXOOOOOOOOOOOOOOOOX |
| | mUfAmCfUmU*fA | A | |
| WV-41056 | Mod001L001mG*mAfUmGfUmAfAfCfAmAfUmCfG | GAUGUAACAAUCGUGGUG | OXOOOOOOOOOOOOOOOOX |
| | mUfGmGfUmG*fA | A | |
| WV-41057 | Mod001L001mA*mGfAmAfGmUfGfGfGmUfGmAfU | AGAAGUGGGUGAUGUAAC | OXOOOOOOOOOOOOOOOOX |
| | mGfUmAfAmC*fA | A | |
| WV-41058 | Mod001L001mU*mAfAmCfAmAfUfCfGmUfGmGfU | UAACAAUCGUGGUGAAUAA | OXOOOOOOOOOOOOOOOOX |
| | mGfAmAfUmA*fA | | |
| WV-41059 | Mod001L001mC*mUfAmGfGmAfCfAfUmUfUmUfU | CUAGGACAUUUUUGGAUC | OXOOOOOOOOOOOOOOOOX |
| | mGfGmAfUmC*fA | A | |
| WV-41060 | Mod001L001mG*mGfAmAfUmAfCfUfUmAfCmCfA | GGAAUACUUACCAAUAAGA | OXOOOOOOOOOOOOOOOOX |
| | mAfUmAfAmG*fA | | |
| WV-41061 | Mod001L001mA*mAfUmCfAmAfUfGfCmUfGmCfA | AAUCAAUGCUGCAAAGCUA | OXOOOOOOOOOOOOOOOOX |
| | mAfAmGfCmU*fA | | |
| WV-41062 | Mod001L001mG*mUfCmUfGmAfCfAfUmCfAmGfA | GUCUGACAUCAGAACUUCA | OXOOOOOOOOOOOOOOOOX |
| | mAfCmUfUmC*fA | | |
| WV-41063 | Mod001L001mC*mAfCmCfAmAfGfGfAmUfGmAfAm | CACCAAGGAUGAAGAGAUA | OXOOOOOOOOOOOOOOOOX |
| | GfAmGfAmU*fA | | |
| WV-41064 | Mod001L001mG*mAfAmUfAmCfUfGfGmGfUmUfC | GAAUACUGGGUUCACCAAA | OXOOOOOOOOOOOOOOOOX |
| | mAfCmCfAmA*fA | | |
| WV-41065 | Mod001L001mC*mAfUmUfUmGfAfGfGmUfCmAfA | CAUUUGAGGUCAACAUCCA | OXOOOOOOOOOOOOOOOOX |
| | mCfAmUfCmC*fA | | |
| WV-41066 | L001mG*mGfCmUfUmUfCfAfCmAfGmAfGmGfUmCf | GGCUUUCACAGAGGUCUGA | OXOOOOOOOOOOOOOOOOX |
| | UmG*fA | | |
| WV-41067 | L001mC*mGfCmUfGmUfUfGfGmCfUmUfUmCfAmCf | CGCUGUUGGCUUUCACAGA | OXOOOOOOOOOOOOOOOOX |
| | AmG*fA | | |
| WV-41068 | L001mC*mUfUmUfCmAfCfAfGmAfGmGfUmCfUmGf | CUUUCACAGAGGUCUGACA | OXOOOOOOOOOOOOOOOOX |
| | AmC*fA | | |
| WV-41069 | L001mG*mGfGmAfAmAfAfAfCmUfGmGfUmAfUmCf | GGGAAAAACUGGUAUCAAA | OXOOOOOOOOOOOOOOOOX |
| | AmA*fA | | |
| WV-41070 | L001mA*mAfGmUfCmUfGfAfUmAfGmAfUmGfGmA | AAGUCUGAUAGAUGGAAU | OXOOOOOOOOOOOOOOOOX |
| | fAmU*fA | A | |
| WV-41071 | L001mG*mGfAmAfAmAfAfCfUmGfGmUfAmUfCmAf | GGAAAAACUGGUAUCAAAA | OXOOOOOOOOOOOOOOOOX |
| | AmA*fA | | |
| WV-41072 | L001mC*mAfAmGfAmUfUfAfUmGfGmCfCmUfGmUf | CAAGAUUAUGGCCUGUAUA | OXOOOOOOOOOOOOOOOOX |
| | AmU*fA | | |
| WV-41073 | L001mG*mGfAmUfCmAfCfAfAmAfAmGfCmAfCmUf | GGAUCACAAAAGCACUUCA | OXOOOOOOOOOOOOOOOOX |
| | UmC*fA | | |
| WV-41074 | L001mC*mAfAmUfAmAfGfAfAmAfAmUfGmAfUmUf | CAAUAAGAAAAUGAUUUUA | OXOOOOOOOOOOOOOOOOX |
| | UmU*fA | | |
| WV-41075 | L001mC*mCfAmUfAmUfUfGfUmUfCmCfAmGfCmAf | CCAUAUUGUUCCAGCAAAA | OXOOOOOOOOOOOOOOOOX |
| | AmA*fA | | |
| WV-41076 | L001mU*mAfGmAfUmGfGfAfAmUfAmCfUmUfAmCf | UAGAUGGAAUACUUACCAA | OXOOOOOOOOOOOOOOOOX |
| | CmA*fA | | |
| WV-41077 | L001mA*mCfAmAfGmAfUfUfAmUfGmGfCmCfUmGf | ACAAGAUUAUGGCCUGUAA | OXOOOOOOOOOOOOOOOOX |
| | UmA*fA | | |
| WV-41078 | L001mC*mCfCmAfUmAfUfUfGmUfUmCfCmAfGmCf | CCCAUAUUGUUCCAGCAAA | OXOOOOOOOOOOOOOOOOX |
| | AmA*fA | | |
| WV-41079 | L001mA*mCfAmGfAmUfGfAfAmGfUmCfGmUfAmAf | ACAGAUGAAGUCGUAAGAA | OXOOOOOOOOOOOOOOOOX |
| | GmA*fA | | |
| WV-41080 | L001mU*mGfUmAfUmUfGfGfAmGfAmCfAmGfAmU | UGUAUUGGAGACAGAUGA | OXOOOOOOOOOOOOOOOOX |
| | fGmA*fA | A | |
| WV-41081 | L001mC*mAfCmAfAmGfAfUfUmAfUmGfGmCfCmUf | CACAAGAUUAUGGCCUGUA | OXOOOOOOOOOOOOOOOOX |
| | GmU*fA | | |
| WV-41082 | L001mG*mAfAmGfUmCfGfUfAmAfGmAfAmGfUmCf | GAAGUCGUAAGAAGUCUGA | OXOOOOOOOOOOOOOOOOX |
| | UmG*fA | | |
| WV-41083 | L001mG*mUfAmUfCmAfAfAfAmCfCmUfCmAfUmGf | GUAUCAAAACCUCAUGUCA | OXOOOOOOOOOOOOOOOOX |
| | UmC*fA | | |
| WV-41084 | L001mG*mAfAmUfAmCfUfUfAmCfCmAfAmUfAmAf | GAAUACUUACCAAUAAGAA | OXOOOOOOOOOOOOOOOOX |
| | GmA*fA | | |
| WV-41085 | L001mA*mGfAmAfGmUfCfUfGmAfUmAfGmAfUmG | AGAAGUCUGAUAGAUGGA | OXOOOOOOOOOOOOOOOOX |
| | fGmA*fA | A | |
| WV-41086 | L001mA*mUfGmGfAmAfUfAfCmUfUmAfCmCfAmAf | AUGGAAUACUUACCAAUAA | OXOOOOOOOOOOOOOOOOX |
| | UmA*fA | | |
| WV-41087 | L001mG*mGfCmCfUmGfUfAfUmUfGmGfAmGfAmC | GGCCUGUAUUGGAGACAG | OXOOOOOOOOOOOOOOOOX |
| | fAmG*fA | A | |
| WV-41088 | L001mG*mGfGmUfUmCfAfCfCmAfAmAfAmAfUmCf | GGGUUCACCAAAAAUCCAA | OXOOOOOOOOOOOOOOOOX |
| | CmA*fA | | |
| WV-41089 | L001mG*mAfCmAfGmAfUfGfAmAfGmUfCmGfUmAf | GACAGAUGAAGUCGUAAGA | OXOOOOOOOOOOOOOOOOX |
| | AmG*fA | | |
| WV-41090 | L001mC*mUfGmAfUmAfGfAfUmGfGmAfAmUfAmCf | CUGAUAGAUGGAAUACUU | OXOOOOOOOOOOOOOOOOX |
| | UmU*fA | A | |
| WV-41091 | L001mG*mAfUmGfUmAfAfCfAmAfUmCfGmUfGmG | GAUGUAACAAUCGUGGUG | OXOOOOOOOOOOOOOOOOX |
| | fUmG*fA | A | |
| WV-41092 | L001mA*mGfAmAfGmUfGfGfGmUfGmAfUmGfUmA | AGAAGUGGGUGAUGUAAC | OXOOOOOOOOOOOOOOOOX |
| | fAmC*fA | A | |
| WV-41093 | L001mU*mAfAmCfAmAfUfCfGmUfGmGfUmGfAmAf | UAACAAUCGUGGUGAAUAA | OXOOOOOOOOOOOOOOOOX |
| | UmA*fA | | |
| WV-41094 | L001mC*mUfAmGfGmAfCfAfUmUfUmUfUmGfGmA | CUAGGACAUUUUUGGAUC | OXOOOOOOOOOOOOOOOOX |
| | fUmC*fA | A | |
| WV-41095 | L001mG*mGfAmAfUmAfCfUfUmAfCmCfAmAfUmAf | GGAAUACUUACCAAUAAGA | OXOOOOOOOOOOOOOOOOX |
| | AmG*fA | | |
| WV-41096 | L001mA*mAfUmCfAmAfUfGfCmUfGmCfAmAfAmGf | AAUCAAUGCUGCAAAGCUA | OXOOOOOOOOOOOOOOOOX |
| | CmU*fA | | |
| WV-41097 | L001mG*mUfCmUfGmAfCfAfUmCfAmGfAmAfCmUf | GUCUGACAUCAGAACUUCA | OXOOOOOOOOOOOOOOOOX |
| | UmC*fA | | |
| WV-41098 | L001mC*mAfCmCfAmAfGfGfAmUfGmAfAmGfAmGf | CACCAAGGAUGAAGAGAUA | OXOOOOOOOOOOOOOOOOX |
| | AmU*fA | | |
| WV-41099 | L001mG*mAfAmUfAmCfUfGfGmGfUmUfCmAfCmCf | GAAUACUGGGUUCACCAAA | OXOOOOOOOOOOOOOOOOX |
| | AmA*fA | | |
| WV-41100 | L001mC*mAfUmUfUmGfAfGfGmUfCmAfAmCfAmUf | CAUUUGAGGUCAACAUCCA | OXOOOOOOOOOOOOOOOOX |
| | CmC*fA | | |
| WV-42129 | fU*fU*mGfCmUfGmCfAmGfUmCmUmAfCmCfAmCf | UUGCUGCAGUCUACCACAU | XXOOOOOOOOOOOOOOOOXX |
| | AmU*mU*mU | UU | |
| WV-42130 | fU*fG*mUfUmGfCmUfGmCfAmGmUmCfUmAfCmCf | UGUUGCUGCAGUCUACCAC | XXOOOOOOOOOOOOOOOOXX |
| | AmC*mU*mU | UU | |
| WV-42131 | fU*fU*mGfUmUfGmCfUmGfCmAmGmUfCmUfAmC | UUGUUGCUGCAGUCUACCA | XXOOOOOOOOOOOOOOOOXX |
| | fCmA*mU*mU | UU | |
| WV-42132 | fU*fC*mUfGmUfUmGfCmUfGmCmAmGfUmCfUmA | UCUGUUGCUGCAGUCUACC | XXOOOOOOOOOOOOOOOOXX |
| | fCmC*mU*mU | UU | |
| WV-42133 | fU*fU*mCfUmGfUmUfGmCfUmGmCmAfGmUfCmU | UUCUGUUGCUGCAGUCUA | XXOOOOOOOOOOOOOOOOXX |
| | fAmC*mU*mU | CUU | |
| WV-42134 | fU*fC*mUfCmUfGmUfUmGfCmUmGmCfAmGfUmCf | UCUCUGUUGCUGCAGUCU | XXOOOOOOOOOOOOOOOOXX |
| | UmA*mU*mU | AUU | |
| WV-42135 | fU*fU*mCfUmCfUmGfUmUfGmCmUmGfCmAfGmU | UUCUCUGUUGCUGCAGUC | XXOOOOOOOOOOOOOOOOXX |
| | fCmU*mU*mU | UUU | |
| WV-42136 | fU*fU*mUfCmUfCmUfGmUfUmGmCmUfGmCfAmG | UUUCUCUGUUGCUGCAGU | XXOOOOOOOOOOOOOOOOXX |
| | fUmC*mU*mU | CUU | |
| WV-42137 | fU*fC*mAfAmAfAmAfUmGfUmCmCmUfAmGfGmAf | UCAAAAAUGUCCUAGGAUG | XXOOOOOOOOOOOOOOOOXX |
| | UmG*mU*mU | UU | |
| WV-42138 | fU*fC*mCfAmAfAmAfAmUfGmUmCmCfUmAfGmGf | UCCAAAAAUGUCCUAGGAU | XXOOOOOOOOOOOOOOOOXX |
| | AmU*mU*mU | UU | |
| WV-42139 | fU*fU*mCfCmAfAmAfAmAfUmGmUmCfCmUfAmGf | UUCCAAAAAUGUCCUAGGA | XXOOOOOOOOOOOOOOOOXX |
| | GmA*mU*mU | UU | |
| WV-42140 | fU*fU*mCfUmGfCmAfUmAfCmGmAmUfUmUfAmAf | UUCUGCAUACGAUUUAAAA | XXOOOOOOOOOOOOOOOOXX |
| | AmA*mU*mU | UU | |
| WV-42141 | fU*fA*mUfUmCfUmGfCmAfUmAmCmGfAmUfUmU | UAUUCUGCAUACGAUUUAA | XXOOOOOOOOOOOOOOOOXX |
| | fAmA*mU*mU | UU | |
| WV-42142 | fU*fA*mUfAmUfUmCfUmGfCmAmUmAfCmGfAmUf | UAUAUUCUGCAUACGAUU | XXOOOOOOOOOOOOOOOOXX |
| | UmU*mU*mU | UUU | |
| WV-42143 | fU*fA*mAfUmAfUmUfCmUfGmCmAmUfAmCfGmAf | UAAUAUUCUGCAUACGAUU | XXOOOOOOOOOOOOOOOOXX |
| | UmU+mU*mU | UU | |
| WV-42144 | fU*fG*mAfAmUfAmUfUmCfUmGmCmAfUmAfCmGf | UGAAUAUUCUGCAUACGAU | XXOOOOOOOOOOOOOOOOXX |
| | AmU*mU*mU | UU | |
| WV-42145 | fU*fU*mGfAmAfUmAfUmUfCmUmGmCfAmUfAmCf | UUGAAUAUUCUGCAUACGA | XXOOOOOOOOOOOOOOOOXX |
| | GmA*mU*mU | UU | |
| WV-42146 | fU*fU*mUfGmAfAmUfAmUfUmCmUmGfCmAfUmA | UUUGAAUAUUCUGCAUAC | XXOOOOOOOOOOOOOOOOXX |
| | fCmG*mU*mU | GUU | |
| WV-42147 | fU*fA*mAfUmUfGmAfAmUfAmUmUmCfUmGfCmAf | UAAUUGAAUAUUCUGCAU | XXOOOOOOOOOOOOOOOOXX |
| | UmA*mU*mU | AUU | |
| WV-42148 | fU*fC*mAfAmAfUmUfGmAfAmUmAmUfUmCfUmGf | UCAAAUUGAAUAUUCUGCA | XXOOOOOOOOOOOOOOOOXX |
| | CmA*mU*mU | UU | |
| WV-42149 | fU*fA*mAfGmUfCmCfAmGfAmAmUmAfGmAfGmUf | UAAGUCCAGAAUAGAGUU | XXOOOOOOOOOOOOOOOOXX |
| | UmG*mU*mU | GUU | |
| WV-42150 | fU*fA*mAfAmGfUmCfCmAfGmAmAmUfAmGfAmGf | UAAAGUCCAGAAUAGAGUU | XXOOOOOOOOOOOOOOOOXX |
| | UmU*mU*mU | UU | |
| WV-42151 | fU*fU*mAfAmAfGmUfCmCfAmGmAmAfUmAfGmAf | UUAAAGUCCAGAAUAGAGU | XXOOOOOOOOOOOOOOOOXX |
| | GmU*mU*mU | UU | |
| WV-42152 | fU*fA*mAfUmAfAmAfGmUfCmCmAmGfAmAfUmAf | UAAUAAAGUCCAGAAUAGA | XXOOOOOOOOOOOOOOOOXX |
| | GmA*mU*mU | UU | |
| WV-42153 | fU*fC*mAfAmGfUmAfAmUfAmAmAmGfUmCfCmAf | UCAAGUAAUAAAGUCCAGA | XXOOOOOOOOOOOOOOOOXX |
| | GmA*mU*mU | UU | |
| WV-42154 | fU*fG*mAfGmGfGmUfCmCfAmCmUmUfUmUfGmG | UGAGGGUCCACUUUUGGU | XXOOOOOOOOOOOOOOOOXX |
| | fUmG*mU*mU | GUU | |
| WV-42155 | fU*fA*mGfAmGfGmGfUmCfCmAmCmUfUmUfUmG | UAGAGGGUCCACUUUUGG | XXOOOOOOOOOOOOOOOOXX |
| | fGmU*mU*mU | UUU | |
| WV-42156 | fU*fU*mAfGmAfGmGfGmUfCmCmAmCfUmUfUmU | UUAGAGGGUCCACUUUUG | XXOOOOOOOOOOOOOOOOXX |
| | fGmG*mU*mU | GUU | |
| WV-42157 | fU*fA*mUfAmGfAmGfGmGfUmCmCmAfCmUfUmUf | UAUAGAGGGUCCACUUUU | XXOOOOOOOOOOOOOOOOXX |
| | UmG*mU*mU | GUU | |
| WV-42158 | fU*fU*mAfUmAfGmAfGmGfGmUmCmCfAmCfUmUf | UUAUAGAGGGUCCACUUU | XXOOOOOOOOOOOOOOOOXX |
| | UmU*mU*mU | UUU | |
| WV-42159 | fU*fA*mAfGmGfGmAfGmGfAmAmAmUfAmUfAmG | UAAGGGAGGAAAUAUAGA | XXOOOOOOOOOOOOOOOOXX |
| | fAmG*mU*mU | GUU | |
| WV-42160 | fU*fU*mGfAmGfCmCfUmAfAmAmAmUfUmGfUmCf | UUGAGCCUAAAAUUGUCUA | XXOOOOOOOOOOOOOOOOXX |
| | UmA*mU*mU | UU | |
| WV-42161 | fU*fU*mUfUmUfGmAfGmCfCmUmAmAfAmAfUmU | UUUUUGAGCCUAAAAUUG | XXOOOOOOOOOOOOOOOOXX |
| | fGmU*mU*mU | UUU | |
| WV-42162 | fU*fA*mCfAmGfUmUfCmCfUmUmUmUfCmCfUmGf | UACAGUUCCUUUUCCUGU | XXOOOOOOOOOOOOOOOOXX |
| | UmG*mU*mU | GUU | |
| WV-42163 | fU*fU*mAfAmUfAmGfCmCfAmGmUmAfCmAfGmUf | UUAAUAGCCAGUACAGUUC | XXOOOOOOOOOOOOOOOOXX |
| | UmC*mU*mU | UU | |
| WV-42164 | fU*fG*mUfAmAfUmAfGmCfCmAmGmUfAmCfAmGf | UGUAAUAGCCAGUACAGUU | XXOOOOOOOOOOOOOOOOXX |
| | UmU*mU*mU | UU | |
| WV-42165 | fU*fA*mGfUmGfGmGfAmGfUmCmGmGfAmUfUmA | UAGUGGGAGUCGGAUUAU | XXOOOOOOOOOOOOOOOOXX |
| | fUmU*mU*mU | UUU | |
| WV-42166 | fU*fG*mUfAmGfUmGfGmGfAmGmUmCfGmGfAmU | UGUAGUGGGAGUCGGAUU | XXOOOOOOOOOOOOOOOOXX |
| | fUmA*mU*mU | AUU | |
| WV-42167 | fU*fU*mGfUmAfGmUfGmGfGmAmGmUfCmGfGmA | UUGUAGUGGGAGUCGGAU | XXOOOOOOOOOOOOOOOOXX |
| | fUmU*mU*mU | UUU | |
| WV-42168 | fU*fG*mAfUmGfUmAfGmUfGmGmGmAfGmUfCmG | UGAUGUAGUGGGAGUCGG | XXOOOOOOOOOOOOOOOOXX |
| | fGmA*mU*mU | AUU | |
| WV-42169 | fU*fU+mGfAmUfGmUfAmGfUmGmGmGfAmGfUmC | UUGAUGUAGUGGGAGUCG | XXOOOOOOOOOOOOOOOOXX |
| | fGmG*mU*mU | GUU | |
| WV-42170 | fU*fU*mUfGmAfUmGfUmAfGmUmGmGfGmAfGm | UUUGAUGUAGUGGGAGUC | XXOOOOOOOOOOOOOOOOXX |
| | UfCmG*mU*mU | GUU | |
| WV-42171 | fU*fC*mUfUmGfAmUfGmUfAmGmUmGfGmGfAmG | UCUUGAUGUAGUGGGAGU | XXOOOOOOOOOOOOOOOOXX |
| | fUmC*mU*mU | CUU | |
| WV-42172 | fU*fU*mCfUmUfGmAfUmGfUmAmGmUfGmGfGmA | UUCUUGAUGUAGUGGGAG | XXOOOOOOOOOOOOOOOOXX |
| | fGmU*mU*mU | UUU | |
| WV-42173 | fU*fG*mUfCmUfUmGfAmUfGmUmAmGfUmGfGmG | UGUCUUGAUGUAGUGGGA | XXOOOOOOOOOOOOOOOOXX |
| | fAmG*mU*mU | GUU | |
| WV-42174 | fU*fA*mGfUmCfUmUfGmAfUmGmUmAfGmUfGmG | UAGUCUUGAUGUAGUGGG | XXOOOOOOOOOOOOOOOOXX |
| | fGmA*mU*mU | AUU | |
| WV-42175 | fU*fU*mUfAmGfUmCfUmUfGmAmUmGfUmAfGmU | UUUAGUCUUGAUGUAGUG | XXOOOOOOOOOOOOOOOOXX |
| | fGmG*mU*mU | GUU | |
| WV-42176 | fU*fA*mUfUmAfGmUfCmUfUmGmAmUfGmUfAmG | UAUUAGUCUUGAUGUAGU | XXOOOOOOOOOOOOOOOOXX |
| | fUmG*mU*mU | GUU | |
| WV-42177 | fU*fG*mAfUmUfAmGfUmCfUmUmGmAfUmGfUmA | UGAUUAGUCUUGAUGUAG | XXOOOOOOOOOOOOOOOOXX |
| | fGmU*mU*mU | UUU | |
| WV-42178 | fU*fA*mGfAmUfUmAfGmUfCmUmUmGfAmUfGmU | UAGAUUAGUCUUGAUGUA | XXOOOOOOOOOOOOOOOOXX |
| | fAmG*mU*mU | GUU | |
| WV-42179 | fU*fC*mAfAmGfAmUfUmAfGmUmCmUfUmGfAmU | UCAAGAUUAGUCUUGAUG | XXOOOOOOOOOOOOOOOOXX |
| | fGmU*mU*mU | UUU | |
| WV-42180 | fU*fA*mCfAmAfGmAfUmUfAmGmUmCfUmUfGmAf | UACAAGAUUAGUCUUGAU | XXOOOOOOOOOOOOOOOOXX |
| | UmG*mU*mU | GUU | |
| WV-42181 | fU*fA+mCfAmUfGmUfGmAfAmAmAmAfCmAfCmAf | UACAUGUGAAAAACACACA | XXOOOOOOOOOOOOOOOOXX |
| | CmA*mU*mU | UU | |
| WV-42182 | fU*fA*mUfAmCfAmUfGmUfGmAmAmAfAmAfCmAf | UAUACAUGUGAAAAACACA | XXOOOOOOOOOOOOOOOOXX |
| | CmA*mU*mU | UU | |
| WV-42183 | fU+fA*mAfUmAfCmAfUmGfUmGmAmAfAmAfAmCf | UAAUACAUGUGAAAAACAC | XXOOOOOOOOOOOOOOOOXX |
| | AmC*mU*mU | UU | |
| WV-42184 | fU*fA*mUfAmAfUmAfCmAfUmGmUmGfAmAfAmAf | UAUAAUACAUGUGAAAAAC | XXOOOOOOOOOOOOOOOOXX |
| | AmC*mU*mU | UU | |
| WV-42185 | fU*fC*mUfAmUfAmAfUmAfCmAmUmGfUmGfAmAf | UCUAUAAUACAUGUGAAAA | XXOOOOOOOOOOOOOOOOXX |
| | AmA*mU*mU | UU | |
| WV-42186 | fU*fU*mCfUmAfUmAfAmUfAmCmAmUfGmUfGmAf | UUCUAUAAUACAUGUGAAA | XXOOOOOOOOOOOOOOOOXX |
| | AmA*mU*mU | UU | |
| WV-42187 | fU*fG*mCfAmUfUmCfUmAfUmAmAmUfAmCfAmUf | UGCAUUCUAUAAUACAUGU | XXOOOOOOOOOOOOOOOOXX |
| | GmU*mU*mU | UU | |
| WV-42188 | fU*fA*mAfGmCfAmUfUmCfUmAmUmAfAmUfAmCf | UAAGCAUUCUAUAAUACAU | XXOOOOOOOOOOOOOOOOXX |
| | AmU*mU*mU | UU | |
| WV-42189 | fU*fC*mAfAmAfAmGfCmAfUmUmCmUfAmUfAmAf | UCAAAAGCAUUCUAUAAUA | XXOOOOOOOOOOOOOOOOXX |
| | UmA*mU*mU | UU | |
| WV-42190 | fU*fA*mUfGmCfAmAfAmAfGmCmAmUfUmCfUmAf | UAUGCAAAAGCAUUCUAUA | XXOOOOOOOOOOOOOOOOXX |
| | UmA*mU*mU | UU | |
| WV-42191 | fU*fC*mCfAmUfGmCfAmAfAmAmGmCfAmUfUmCf | UCCAUGCAAAAGCAUUCUA | XXOOOOOOOOOOOOOOOOXX |
| | UmA*mU*mU | UU | |
| WV-42192 | fU*fU*mCfCmAfUmGfCmAfAmAmAmGfCmAfUmUf | UUCCAUGCAAAAGCAUUCU | XXOOOOOOOOOOOOOOOOXX |
| | CmU*mU*mU | UU | |
| WV-42193 | fU*fG*mUfCmCfAmUfGmCfAmAmAmAfGmCfAmUf | UGUCCAUGCAAAAGCAUUC | XXOOOOOOOOOOOOOOOOXX |
| | UmC*mU*mU | UU | |
| WV-42194 | fU*fA*mGfUmCfCmAfUmGfCmAmAmAfAmGfCmAf | UAGUCCAUGCAAAAGCAUU | XXOOOOOOOOOOOOOOOOXX |
| | UmU*mU*mU | UU | |
| WV-42195 | fU*fU*mAfGmUfCmCfAmUfGmCmAmAfAmAfGmCf | UUAGUCCAUGCAAAAGCAU | XXOOOOOOOOOOOOOOOOXX |
| | AmU*mU*mU | UU | |
| WV-42196 | fU*fG*mAfUmAfGmUfCmCfAmUmGmCfAmAfAmAf | UGAUAGUCCAUGCAAAAGC | XXOOOOOOOOOOOOOOOOXX |
| | GmC*mU*mU | UU | |
| WV-42197 | fU*fG*mGfAmUfAmGfUmCfCmAmUmGfCmAfAmAf | UGGAUAGUCCAUGCAAAAG | XXOOOOOOOOOOOOOOOOXX |
| | AmG*mU*mU | UU | |
| WV-42198 | fU*fA*mGfGmAfUmAfGmUfCmCmAmUfGmCfAmAf | UAGGAUAGUCCAUGCAAAA | XXOOOOOOOOOOOOOOOOXX |
| | AmA*mU*mU | UU | |
| WV-42199 | fU*fA*mGfAmGfGmAfUmAfGmUmCmCfAmUfGmCf | UAGAGGAUAGUCCAUGCAA | XXOOOOOOOOOOOOOOOOXX |
| | AmA*mU*mU | UU | |
| WV-42200 | fU*fA*mAfGmAfGmGfAmUfAmGmUmCfCmAfUmGf | UAAGAGGAUAGUCCAUGCA | XXOOOOOOOOOOOOOOOOXX |
| | CmA*mU*mU | UU | |
| WV-42201 | fU*fC*mAfAmGfAmGfGmAfUmAmGmUfCmCfAmUf | UCAAGAGGAUAGUCCAUGC | XXOOOOOOOOOOOOOOOOXX |
| | GmC*mU*mU | UU | |
| WV-42202 | fU*fA*mCfAmAfGmAfGmGfAmUmAmGfUmCfCmAf | UACAAGAGGAUAGUCCAUG | XXOOOOOOOOOOOOOOOOXX |
| | UmG*mU*mU | UU | |
| WV-42203 | fU*fA*mAfCmAfAmGfAmGfGmAmUmAfGmUfCmCf | UAACAAGAGGAUAGUCCAU | XXOOOOOOOOOOOOOOOOXX |
| | AmU*mU*mU | UU | |
| WV-42204 | fU*fA*mAfAmCfAmAfGmAfGmGmAmUfAmGfUmCf | UAAACAAGAGGAUAGUCCA | XXOOOOOOOOOOOOOOOOXX |
| | CmA*mU*mU | UU | |
| WV-42205 | fU*fA*mAfAmAfAmCfAmAfGmAmGmGfAmUfAmGf | UAAAAACAAGAGGAUAGUC | XXOOOOOOOOOOOOOOOOXX |
| | UmC*mU*mU | UU | |
| WV-42206 | fU*fA*mAfUmAfAmAfAmAfCmAmAmGfAmGfGmAf | UAAUAAAAACAAGAGGAUA | XXOOOOOOOOOOOOOOOOXX |
| | UmA*mU*mU | UU | |
| WV-42207 | fU*fU*mAfAmUfAmAfAmAfAmCmAmAfGmAfGmGf | UUAAUAAAAACAAGAGGAU | XXOOOOOOOOOOOOOOOOXX |
| | AmU*mU*mU | UU | |
| WV-42208 | fU*fU*mUfAmAfUmAfAmAfAmAmCmAfAmGfAmGf | UUUAAUAAAAACAAGAGGA | XXOOOOOOOOOOOOOOOOXX |
| | GmA*mU*mU | UU | |
| WV-42209 | fU*fC*mAfUmUfUmGfUmUfUmUmUmAfAmUfAmA | UCAUUUGUUUUUAAUAAA | XXOOOOOOOOOOOOOOOOXX |
| | fAmA*mU*mU | AUU | |
| WV-42210 | fU*fA*mAfUmCfAmUfUmUfGmUmUmUfUmUfAmA | UAAUCAUUUGUUUUUAAU | XXOOOOOOOOOOOOOOOOXX |
| | fUmA*mU*mU | AUU | |
| WV-42211 | fU*fA*mAfAmUfCmAfUmUfUmGmUmUfUmUfUmA | UAAAUCAUUUGUUUUUAA | XXOOOOOOOOOOOOOOOOXX |
| | fAmU*mU*mU | UUU | |
| WV-42212 | fU*fA*mCfUmAfCmAfAmGfAmGmGmUfUmAfUmUf | UACUACAAGAGGUUAUUU | XXOOOOOOOOOOOOOOOOXX |
| | UmU*mU*mU | UUU | |
| WV-42213 | fU*fA+mAfCmUfAmCfAmAfGmAmGmGfUmUfAmUf | UAACUACAAGAGGUUAUU | XXOOOOOOOOOOOOOOOOXX |
| | UmU*mU*mU | UUU | |
| WV-42214 | fU*fU*mAfAmCfUmAfCmAfAmGmAmGfGmUfUmAf | UUAACUACAAGAGGUUAU | XXOOOOOOOOOOOOOOOOXX |
| | UmU*mU*mU | UUU | |
| WV-42215 | fU*fA*mUfAmAfCmUfAmCfAmAmGmAfGmGfUmUf | UAUAACUACAAGAGGUUAU | XXOOOOOOOOOOOOOOOOXX |
| | AmU*mU*mU | UU | |
| WV-42216 | fU*fU*mAfUmAfAmCfUmAfCmAmAmGfAmGfGmUf | UUAUAACUACAAGAGGUUA | XXOOOOOOOOOOOOOOOOXX |
| | UmA*mU*mU | UU | |
| WV-42217 | fU*fU*mUfAmUfAmAfCmUfAmCmAmAfGmAfGmGf | UUUAUAACUACAAGAGGU | XXOOOOOOOOOOOOOOOOXX |
| | UmU*mU*mU | UUU | |
| WV-42218 | fU*fA*mCfGmUfUmUfUmAfUmUmUmUfAmUfAmA | UACGUUUUAUUUUAUAAC | XXOOOOOOOOOOOOOOOOXX |
| | fCmU*mU*mU | UUU | |
| WV-42219 | mAmUfGmUfGmGfUfAfGmAfCmUfGmCfAmGfCmAf | AUGUGGUAGACUGCAGCAA | OOOOOOOOOOOOOOOOOO |
| | A | | |
| WV-42220 | mGmUfGmGfUmAfGfAfCmUfGmCfAmGfCmAfAmCf | GUGGUAGACUGCAGCAACA | OOOOOOOOOOOOOOOOOO |
| | A | | |
| WV-42221 | mUmGfGmUfAmGfAfCfUmGfCmAfGmCfAmAfCmAf | UGGUAGACUGCAGCAACAA | OOOOOOOOOOOOOOOOOO |
| | A | | |
| WV-42222 | mGmGfUmAfGmAfCfUfGmCfAmGfCmAfAmCfAmGf | GGUAGACUGCAGCAACAGA | OOOOOOOOOOOOOOOOOO |
| | A | | |
| WV-42223 | mGmUfAmGfAmCfUfGfCmAfGmCfAmAfCmAfGmAf | GUAGACUGCAGCAACAGAA | OOOOOOOOOOOOOOOOOO |
| | A | | |
| WV-42224 | mUmAfGmAfCmUfGfCfAmGfCmAfAmCfAmGfAmGf | UAGACUGCAGCAACAGAGA | OOOOOOOOOOOOOOOOOO |
| | A | | |
| WV-42225 | mAmGfAmCfUmGfCfAfGmCfAmAfCmAfGmAfGmAf | AGACUGCAGCAACAGAGAA | OOOOOOOOOOOOOOOOOO |
| | A | | |
| WV-42226 | mGmAfCmUfGmCfAfGfCmAfAmCfAmGfAmGfAmAf | GACUGCAGCAACAGAGAAA | OOOOOOOOOOOOOOOOOO |
| | A | | |
| WV-42227 | mCmAfUmCfCmUfAfGfGmAfCmAfUmUfUmUfUmGf | CAUCCUAGGACAUUUUUGA | OOOOOOOOOOOOOOOOOO |
| | A | | |
| WV-42228 | mAmUfCmCfUmAfGfGfAmCfAmUfUmUfUmUfGmGf | AUCCUAGGACAUUUUUGG | OOOOOOOOOOOOOOOOOO |
| | A | A | |
| WV-42229 | mUmCfCmUfAmGfGfAfCmAfUmUfUmUfUmGfGmAf | UCCUAGGACAUUUUUGGA | OOOOOOOOOOOOOOOOOO |
| | A | A | |
| WV-42230 | mUmUfUmUfAmAfAfUfCmGfUmAfUmGfCmAfGmAf | UUUUAAAUCGUAUGCAGA | OOOOOOOOOOOOOOOOOO |
| | A | A | |
| WV-42231 | mUmUfAmAfAmUfCfGfUmAfUmGfCmAfGmAfAmUf | UUAAAUCGUAUGCAGAAUA | OOOOOOOOOOOOOOOOOO |
| | A | | |
| WV-42232 | mAmAfAmUfCmGfUfAfUmGfCmAfGmAfAmUfAmUf | AAAUCGUAUGCAGAAUAUA | OOOOOOOOOOOOOOOOOO |
| | A | | |
| WV-42233 | mAmAfUmCfGmUfAfUfGmCfAmGfAmAfUmAfUmUf | AAUCGUAUGCAGAAUAUUA | OOOOOOOOOOOOOOOOOO |
| | A | | |
| WV-42234 | mAmUfCmGfUmAfUfGfCmAfGmAfAmUfAmUfUmCf | AUCGUAUGCAGAAUAUUCA | OOOOOOOOOOOOOOOOOO |
| | A | | |
| WV-42235 | mUmCfGmUfAmUfGfCfAmGfAmAfUmAfUmUfCmAf | UCGUAUGCAGAAUAUUCAA | OOOOOOOOOOOOOOOOOO |
| | A | | |
| WV-42236 | mCmGfUmAfUmGfCfAfGmAfAmUfAmUfUmCfAmAf | CGUAUGCAGAAUAUUCAAA | OOOOOOOOOOOOOOOOOO |
| | A | | |
| WV-42237 | mUmAfUmGfCmAfGfAfAmUfAmUfUmCfAmAfUmUf | UAUGCAGAAUAUUCAAUUA | OOOOOOOOOOOOOOOOOO |
| | A | | |
| WV-42238 | mUmGfCmAfGmAfAfUfAmUfUmCfAmAfUmUfUmGf | UGCAGAAUAUUCAAUUUG | OOOOOOOOOOOOOOOOOO |
| | A | A | |
| WV-42239 | mCmAfAmCfUmCfUfAfUmUfCmUfGmGfAmCfUmUf | CAACUCUAUUCUGGACUUA | OOOOOOOOOOOOOOOOOO |
| | A | | |
| WV-42240 | mAmAfCmUfCmUfAfUfUmCfUmGfGmAfCmUfUmUf | AACUCUAUUCUGGACUUUA | OOOOOOOOOOOOOOOOOO |
| | A | | |
| WV-42241 | mAmCfUmCfUmAfUfUfCmUfGmGfAmCfUmUfUmAf | ACUCUAUUCUGGACUUUAA | OOOOOOOOOOOOOOOOOO |
| | A | | |
| WV-42242 | mUmCfUmAfUmUfCfUfGmGfAmCfUmUfUmAfUmU | UCUAUUCUGGACUUUAUU | OOOOOOOOOOOOOOOOOO |
| | fA | A | |
| WV-42243 | mUmCfUmGfGmAfCfUfUmUfAmUfUmAfCmUfUmG | UCUGGACUUUAUUACUUG | OOOOOOOOOOOOOOOOOO |
| | fA | A | |
| WV-42244 | mCmAfCmCfAmAfAfAfGmUfGmGfAmCfCmCfUmCf | CACCAAAAGUGGACCCUCA | OOOOOOOOOOOOOOOOOO |
| | A | | |
| WV-42245 | mAmCfCmAfAmAfAfGfUmGfGmAfCmCfCmUfCmUf | ACCAAAAGUGGACCCUCUA | OOOOOOOOOOOOOOOOOO |
| | A | | |
| WV-42246 | mCmCfAmAfAmAfGfUfGmGfAmCfCmCfUmCfUmAf | CCAAAAGUGGACCCUCUAA | OOOOOOOOOOOOOOOOOO |
| | A | | |
| WV-42247 | mCmAfAmAfAmGfUfGfGmAfCmCfCmUfCmUfAmUf | CAAAAGUGGACCCUCUAUA | OOOOOOOOOOOOOOOOOO |
| | A | | |
| WV-42248 | mAmAfAmAfGmUfGfGfAmCfCmCfUmCfUmAfUmAf | AAAAGUGGACCCUCUAUAA | OOOOOOOOOOOOOOOOOO |
| | A | | |
| WV-42249 | mCmUfCmUfAmUfAfUfUmUfCmCfUmCfCmCfUmUf | CUCUAUAUUUCCUCCCUUA | OOOOOOOOOOOOOOOOOO |
| | A | | |
| WV-42250 | mUmAfGmAfCmAfAfUfUmUfUmAfGmGfCmUfCmAf | UAGACAAUUUUAGGCUCAA | OOOOOOOOOOOOOOOOOO |
| | A | | |
| WV-42251 | mAmCfAmAfUmUfUfUfAmGfGmCfUmCfAmAfAmAf | ACAAUUUUAGGCUCAAAAA | OOOOOOOOOOOOOOOOOO |
| | A | | |
| WV-42252 | mCmAfCmAfGmGfAfAfAmAfGmGfAmAfCmUfGmUf | CACAGGAAAAGGAACUGUA | OOOOOOOOOOOOOOOOOO |
| | A | | |
| WV-42253 | mGmAfAmCfUmGfUfAfCmUfGmGfCmUfAmUfUmAf | GAACUGUACUGGCUAUUAA | OOOOOOOOOOOOOOOOOO |
| | A | | |
| WV-42254 | mAmAfCmUfGmUfAfCfUmGfGmCfUmAfUmUfAmCf | AACUGUACUGGCUAUUACA | OOOOOOOOOOOOOOOOOO |
| | A | | |
| WV-42255 | mAmAfUmAfAmUfCfCfGmAfCmUfCmCfCmAfCmUf | AAUAAUCCGACUCCCACUA | OOOOOOOOOOOOOOOOOO |
| | A | | |
| WV-42256 | mUmAfAmUfCmCfGfAfCmUfCmCfCmAfCmUfAmCf | UAAUCCGACUCCCACUACA | OOOOOOOOOOOOOOOOOO |
| | A | | |
| WV-42257 | mAmAfUmCfCmGfAfCfUmCfCmCfAmCfUmAfCmAf | AAUCCGACUCCCACUACAA | OOOOOOOOOOOOOOOOOO |
| | A | | |
| WV-42258 | mUmCfCmGfAmCfUfCfCmCfAmCfUmAfCmAfUmCf | UCCGACUCCCACUACAUCA | OOOOOOOOOOOOOOOOOO |
| | A | | |
| WV-42259 | mCmCfGmAfCmUfCfCfCmAfCmUfAmCfAmUfCmAf | CCGACUCCCACUACAUCAA | OOOOOOOOOOOOOOOOOO |
| | A | | |
| WV-42260 | mCmGfAmCfUmCfCfCfAmCfUmAfCmAfUmCfAmAf | CGACUCCCACUACAUCAAA | OOOOOOOOOOOOOOOOOO |
| | A | | |
| WV-42261 | mGmAfCmUfCmCfCfAfCmUfAmCfAmUfCmAfAmGf | GACUCCCACUACAUCAAGA | OOOOOOOOOOOOOOOOOO |
| | A | | |
| WV-42262 | mAmCfUmCfCmCfAfCfUmAfCmAfUmCfAmAfGmAf | ACUCCCACUACAUCAAGAA | OOOOOOOOOOOOOOOOOO |
| | A | | |
| WV-42263 | mCmUfCmCfCmAfCfUfAmCfAmUfCmAfAmGfAmCf | CUCCCACUACAUCAAGACA | OOOOOOOOOOOOOOOOOO |
| | A | | |
| WV-42264 | mUmCfCmCfAmCfUfAfCmAfUmCfAmAfGmAfCmUf | UCCCACUACAUCAAGACUA | OOOOOOOOOOOOOOOOOO |
| | A | | |
| WV-42265 | mCmCfAmCfUmAfCfAfUmCfAmAfGmAfCmUfAmAf | CCACUACAUCAAGACUAAA | OOOOOOOOOOOOOOOOOO |
| | A | | |
| WV-42266 | mCmAfCmUfAmCfAfUfCmAfAmGfAmCfUmAfAmUf | CACUACAUCAAGACUAAUA | OOOOOOOOOOOOOOOOOO |
| | A | | |
| WV-42267 | mAmCfUmAfCmAfUfCfAmAfGmAfCmUfAmAfUmCf | ACUACAUCAAGACUAAUCA | OOOOOOOOOOOOOOOOOO |
| | A | | |
| WV-42268 | mCmUfAmCfAmUfCfAfAmGfAmCfUmAfAmUfCmUf | CUACAUCAAGACUAAUCUA | OOOOOOOOOOOOOOOOOO |
| | A | | |
| WV-42269 | mAmCfAmUfCmAfAfGfAmCfUmAfAmUfCmUfUmGf | ACAUCAAGACUAAUCUUGA | OOOOOOOOOOOOOOOOOO |
| | A | | |
| WV-42270 | mCmAfUmCfAmAfGfAfCmUfAmAfUmCfUmUfGmUf | CAUCAAGACUAAUCUUGUA | OOOOOOOOOOOOOOOOOO |
| | A | | |
| WV-42271 | mUmGfUmGfUmGfUfUfUmUfUmCfAmCfAmUfGmU | UGUGUGUUUUUCACAUGU | OOOOOOOOOOOOOOOOOO |
| | fA | A | |
| WV-42272 | mUmGfUmGfUmUfUfUfUmCfAmCfAmUfGmUfAmU | UGUGUUUUUCACAUGUAU | OOOOOOOOOOOOOOOOOO |
| | fA | A | |
| WV-42273 | mGmUfGmUfUmUfUfUfCmAfCmAfUmGfUmAfUmU | GUGUUUUUCACAUGUAUU | OOOOOOOOOOOOOOOOOO |
| | fA | A | |
| WV-42274 | mGmUfUmUfUmUfCfAfCmAfUmGfUmAfUmUfAmU | GUUUUUCACAUGUAUUAU | OOOOOOOOOOOOOOOOOO |
| | fA | A | |
| WV-42275 | mUmUfUmUfCmAfCfAfUmGfUmAfUmUfAmUfAmG | UUUUCACAUGUAUUAUAG | OOOOOOOOOOOOOOOOOO |
| | fA | A | |
| WV-42276 | mUmUfUmCfAmCfAfUfGmUfAmUfUmAfUmAfGmAf | UUUCACAUGUAUUAUAGA | OOOOOOOOOOOOOOOOOO |
| | A | A | |
| WV-42277 | mAmCfAmUfGmUfAfUfUmAfUmAfGmAfAmUfGmCf | ACAUGUAUUAUAGAAUGCA | OOOOOOOOOOOOOOOOOO |
| | A | | |
| WV-42278 | mAmUfGmUfAmUfUfAfUmAfGmAfAmUfGmCfUmU | AUGUAUUAUAGAAUGCUU | OOOOOOOOOOOOOOOOOO |
| | fA | A | |
| WV-42279 | mUmAfUmUfAmUfAfGfAmAfUmGfCmUfUmUfUmG | UAUUAUAGAAUGCUUUUG | OOOOOOOOOOOOOOOOOO |
| | fA | A | |
| WV-42280 | mUmAfUmAfGmAfAfUfGmCfUmUfUmUfGmCfAmU | UAUAGAAUGCUUUUGCAU | OOOOOOOOOOOOOOOOOO |
| | fA | A | |
| WV-42281 | mUmAfGmAfAmUfGfCfUmUfUmUfGmCfAmUfGmG | UAGAAUGCUUUUGCAUGG | OOOOOOOOOOOOOOOOOO |
| | fA | A | |
| WV-42282 | mAmGfAmAfUmGfCfUfUmUfUmGfCmAfUmGfGmAf | AGAAUGCUUUUGCAUGGA | OOOOOOOOOOOOOOOOOO |
| | A | A | |
| WV-42283 | mGmAfAmUfGmCfUfUfUmUfGmCfAmUfGmGfAmCf | GAAUGCUUUUGCAUGGAC | OOOOOOOOOOOOOOOOOO |
| | A | A | |
| WV-42284 | mAmAfUmGfCmUfUfUfUmGfCmAfUmGfGmAfCmUf | AAUGCUUUUGCAUGGACU | OOOOOOOOOOOOOOOOOO |
| | A | A | |
| WV-42285 | mAmUfGmCfUmUfUfUfGmCfAmUfGmGfAmCfUmAf | AUGCUUUUGCAUGGACUA | OOOOOOOOOOOOOOOOOO |
| | A | A | |
| WV-42286 | mGmCfUmUfUmUfGfCfAmUfGmGfAmCfUmAfUmCf | GCUUUUGCAUGGACUAUCA | OOOOOOOOOOOOOOOOOO |
| | A | | |
| WV-42287 | mCmUfUmUfUmGfCfAfUmGfGmAfCmUfAmUfCmCf | CUUUUGCAUGGACUAUCCA | OOOOOOOOOOOOOOOOOO |
| | A | | |
| WV-42288 | mUmUfUmUfGmCfAfUfGmGfAmCfUmAfUmCfCmUf | UUUUGCAUGGACUAUCCUA | OOOOOOOOOOOOOOOOOO |
| | A | | |
| WV-42289 | mUmUfGmCfAmUfGfGfAmCfUmAfUmCfCmUfCmUf | UUGCAUGGACUAUCCUCUA | OOOOOOOOOOOOOOOOOO |
| | A | | |
| WV-42290 | mUmGfCmAfUmGfGfAfCmUfAmUfCmCfUmCfUmUf | UGCAUGGACUAUCCUCUUA | OOOOOOOOOOOOOOOOOO |
| | A | | |
| WV-42291 | mGmCfAmUfGmGfAfCfUmAfUmCfCmUfCmUfUmGf | GCAUGGACUAUCCUCUUGA | OOOOOOOOOOOOOOOOOO |
| | A | | |
| WV-42292 | mCmAfUmGfGmAfCfUfAmUfCmCfUmCfUmUfGmUf | CAUGGACUAUCCUCUUGUA | OOOOOOOOOOOOOOOOOO |
| | A | | |
| WV-42293 | mAmUfGmGfAmCfUfAfUmCfCmUfCmUfUmGfUmUf | AUGGACUAUCCUCUUGUUA | OOOOOOOOOOOOOOOOOO |
| | A | | |
| WV-42294 | mUmGfGmAfCmUfAfUfCmCfUmCfUmUfGmUfUmUf | UGGACUAUCCUCUUGUUU | OOOOOOOOOOOOOOOOOO |
| | A | A | |
| WV-42295 | mGmAfCmUfAmUfCfCfUmCfUmUfGmUfUmUfUmUf | GACUAUCCUCUUGUUUUU | OOOOOOOOOOOOOOOOOO |
| | A | A | |
| WV-42296 | mUmAfUmCfCmUfCfUfUmGfUmUfUmUfUmAfUmU | UAUCCUCUUGUUUUUAUU | OOOOOOOOOOOOOOOOOO |
| | fA | A | |
| WV-42297 | mAmUfCmCfUmCfUfUfGmUfUmUfUmUfAmUfUmA | AUCCUCUUGUUUUUAUUA | OOOOOOOOOOOOOOOOOO |
| | fA | A | |
| WV-42298 | mUmCfCmUfCmUfUfGfUmUfUmUfUmAfUmUfAmA | UCCUCUUGUUUUUAUUAA | OOOOOOOOOOOOOOOOOO |
| | fA | A | |
| WV-42299 | mUmUfUmUfAmUfUfAfAmAfAmAfCmAfAmAfUmGf | UUUUAUUAAAAACAAAUGA | OOOOOOOOOOOOOOOOOO |
| | A | | |
| WV-42300 | mUmAfUmUfAmAfAfAfAmCfAmAfAmUfGmAfUmUf | UAUUAAAAACAAAUGAUUA | OOOOOOOOOOOOOOOOOO |
| | A | | |
| WV-42301 | mAmUfUmAfAmAfAfAfCmAfAmAfUmGfAmUfUmUf | AUUAAAAACAAAUGAUUUA | OOOOOOOOOOOOOOOOOO |
| | A | | |
| WV-42302 | mAmAfAmAfUmAfAfCfCmUfCmUfUmGfUmAfGmUf | AAAAUAACCUCUUGUAGUA | OOOOOOOOOOOOOOOOOO |
| | A | | |
| WV-42303 | mAmAfAmUfAmAfCfCfUmCfUmUfGmUfAmGfUmUf | AAAUAACCUCUUGUAGUUA | OOOOOOOOOOOOOOOOOO |
| | A | | |
| WV-42304 | mAmAfUmAfAmCfCfUfCmUfUmGfUmAfGmUfUmAf | AAUAACCUCUUGUAGUUAA | OOOOOOOOOOOOOOOOOO |
| | A | | |
| WV-42305 | mAmUfAmAfCmCfUfCfUmUfGmUfAmGfUmUfAmUf | AUAACCUCUUGUAGUUAUA | OOOOOOOOOOOOOOOOOO |
| | A | | |
| WV-42306 | mUmAfAmCfCmUfCfUfUmGfUmAfGmUfUmAfUmAf | UAACCUCUUGUAGUUAUAA | OOOOOOOOOOOOOOOOOO |
| | A | | |
| WV-42307 | mAmAfCmCfUmCfUfUfGmUfAmGfUmUfAmUfAmAf | AACCUCUUGUAGUUAUAAA | OOOOOOOOOOOOOOOOOO |
| | A | | |
| WV-42308 | mAmGfUmUfAmUfAfAfAmAfUmAfAmAfAmCfGmUf | AGUUAUAAAAUAAAACGUA | OOOOOOOOOOOOOOOOOO |
| | A | | |
| WV-42354 | mU*fG*mAmUmGfUmUmCmAmUmGmGmCfUmUf | UGAUGUUCAUGGCUUUGC | XXOOOOOOOOOOOOOOOOOO |
| | UmGmCmUmCmU*mU*mU | UCUUU | XX |
| WV-42355 | mU*fA*mGmGmAfUmGmAmUmGmUmUmCfAmUf | UAGGAUGAUGUUCAUGGC | XXOOOOOOOOOOOOOOOOOO |
| | GmGmCmUmUmU*mU*mU | UUUUU | XX |
| WV-42356 | mU*fU*mUmUmCfUmAmGmGmAmUmGmAfUmGf | UUUUCUAGGAUGAUGUUC | XXOOOOOOOOOOOOOOOOOO |
| | UmUmCmAmUmG*mU*mU | AUGUU | XX |
| WV-42357 | mU*fA*mGmGmAfUmUmUmCmUmAmGmGfAmUf | UAGGAUUUCUAGGAUGAU | XXOOOOOOOOOOOOOOOOOO |
| | GmAmUmGmUmU*mU*mU | GUUUU | XX |
| WV-42358 | mU*fA*mGmCmAfGmAmAmGmGmAmUmUfUmCf | UAGCAGAAGGAUUUCUAG | XXOOOOOOOOOOOOOOOOOO |
| | UmAmGmGmAmU*mU*mU | GAUUU | XX |
| WV-42359 | mU*fG*mAmAmGfCmAmGmAmAmGmGmAfUmUf | UGAAGCAGAAGGAUUUCU | XXOOOOOOOOOOOOOOOOOO |
| | UmCmUmAmGmG*mU*mU | AGGUU | XX |
| WV-42360 | mU*fA*mUmGmAfUmGmGmUmGmAmUmCfAmGf | UAUGAUGGUGAUCAGAAG | XXOOOOOOOOOOOOOOOOOO |
| | AmAmGmCmAmG*mU*mU | CAGUU | XX |
| WV-42361 | mU*fA*mGmAmUfGmAmUmGmGmUmGmAfUmCf | UAGAUGAUGGUGAUCAGA | XXOOOOOOOOOOOOOOOOOO |
| | AmGmAmAmGmC*mU*mU | AGCUU | XX |
| WV-42362 | mU*fG*mUmAmAfUmGmAmGmAmAmCmAfAmUf | UGUAAUGAGAACAAUCUCC | XXOOOOOOOOOOOOOOOOOO |
| | CmUmCmCmCmC*mU*mU | CCUU | XX |
| WV-42363 | mU*fA*mGmUmAfAmUmGmAmGmAmAmCfAmAf | UAGUAAUGAGAACAAUCUC | XXOOOOOOOOOOOOOOOOOO |
| | UmCmUmCmCmC*mU*mU | CCUU | XX |
| WV-42364 | mU*fG*mUmUmAfCmAmUmCmAmCmCmCfAmCfU | UGUUACAUCACCCACUUCU | XXOOOOOOOOOOOOOOOOOO |
| | mUmCmUmUmU*mU*mU | UUUU | XX |
| WV-42365 | mU*fC*mAmCmCfAmCmGmAmUmUmGmUfUmAf | UCACCACGAUUGUUACAUC | XXOOOOOOOOOOOOOOOOOO |
| | CmAmUmCmAmC*mU*mU | ACUU | XX |
| WV-42366 | mU*fU*mAmUmUfCmAmCmCmAmCmGmAfUmUf | UUAUUCACCACGAUUGUUA | XXOOOOOOOOOOOOOOOOOO |
| | GmUmUmAmCmA*mU*mU | CAUU | XX |
| WV-42367 | mU*fA*mGmAmUfCmGmGmCmUmGmGmAfUmAf | UAGAUCGGCUGGAUAUAC | XXOOOOOOOOOOOOOOOOOO |
| | UmAmCmUmGmU*mU*mU | UGUUU | XX |
| WV-42368 | mU*fU*mCmUmCfUmUmCmAmUmCmCmUfUmGf | UUCUCUUCAUCCUUGGUGC | XXOOOOOOOOOOOOOOOOOO |
| | GmUmGmCmUmG*mU*mU | UGUU | XX |
| WV-42369 | mU*fA*mUmCmUfCmUmUmCmAmUmCmCfUmUf | UAUCUCUUCAUCCUUGGU | XXOOOOOOOOOOOOOOOOOO |
| | GmGmUmGmCmU*mU*mU | GCUUU | XX |
| WV-42370 | mU*fG*mGmUmAfAmUmCmUmCmUmUmCfAmUf | UGGUAAUCUCUUCAUCCUU | XXOOOOOOOOOOOOOOOOOO |
| | CmCmUmUmGmG*mU*mU | GGUU | XX |
| WV-42371 | mU*fG*mGmAmUfGmUmUmGmAmCmCmUfCmAf | UGGAUGUUGACCUCAAAU | XXOOOOOOOOOOOOOOOOOO |
| | AmAmUmGmUmC*mU*mU | GUCUU | XX |
| WV-42372 | mU*fG*mAmUmCfCmAmAmAmAmAmUmGfUmCf | UGAUCCAAAAAUGUCCUAG | XXOOOOOOOOOOOOOOOOOO |
| | CmUmAmGmGmA*mU*mU | GAUU | XX |
| WV-42373 | mU*fG*mUmGmAfUmCmCmAmAmAmAmAfUmGf | UGUGAUCCAAAAAUGUCCU | XXOOOOOOOOOOOOOOOOOO |
| | UmCmCmUmAmG*mU*mU | AGUU | XX |
| WV-42374 | mU*fG*mAmAmGfUmGmCmUmUmUmUmGfUmGf | UGAAGUGCUUUUGUGAUC | XXOOOOOOOOOOOOOOOOOO |
| | AmUmCmCmAmA*mU*mU | CAAUU | XX |
| WV-42375 | mU*fU*mGmGmAfAmGmAmAmGmUmGmCfUmUf | UUGGAAGAAGUGCUUUUG | XXOOOOOOOOOOOOOOOOOO |
| | UmUmGmUmGmA*mU*mU | UGAUU | XX |
| WV-42376 | mU*fU*mUmGmCfUmGmGmAmAmCmAmAfUmAf | UUUGCUGGAACAAUAUGG | XXOOOOOOOOOOOOOOOOOO |
| | UmGmGmGmAmU*mU*mU | GAUUU | XX |
| WV-42377 | mU*fC*mUmGmUfGmAmAmAmGmCmCmAfAmCfA | UCUGUGAAAGCCAACAGCG | XXOOOOOOOOOOOOOOOOOO |
| | mGmCmGmGmC*mU*mU | GCUU | XX |
| WV-42378 | mU*fC*mAmGmAfCmCmUmCmUmGmUmGfAmAf | UCAGACCUCUGUGAAAGCC | XXOOOOOOOOOOOOOOOOOO |
| | AmGmCmCmAmA*mU*mU | AAUU | XX |
| WV-42379 | mU*fG*mUmCmAfGmAmCmCmUmCmUmGfUmGf | UGUCAGACCUCUGUGAAAG | XXOOOOOOOOOOOOOOOOOO |
| | AmAmAmGmCmC*mU*mU | CCUU | XX |
| WV-42380 | mU*fG*mAmAmGfUmUmCmUmGmAmUmGfUmCf | UGAAGUUCUGAUGUCAGA | XXOOOOOOOOOOOOOOOOOO |
| | AmGmAmCmCmU*mU*mU | CCUUU | XX |
| WV-42381 | mU*fU*mUmGmAfUmAmCmCmAmGmUmUfUmUf | UUUGAUACCAGUUUUUCCC | XXOOOOOOOOOOOOOOOOOO |
| | UmCmCmCmAmA*mU*mU | AAUU | XX |
| WV-42382 | mU*fU*mUmUmGfAmUmAmCmCmAmGmUfUmUf | UUUUGAUACCAGUUUUUC | XXOOOOOOOOOOOOOOOOOO |
| | UmUmCmCmCmA*mU*mU | CCAUU | XX |
| WV-42383 | mU*fG*mAmCmAfUmGmAmGmGmUmUmUfUmGf | UGACAUGAGGUUUUGAUA | XXOOOOOOOOOOOOOOOOOO |
| | AmUmAmCmCmA*mU*mU | CCAUU | XX |
| WV-42384 | mU*fA*mCmAmGfGmCmCmAmUmAmAmUfCmUf | UACAGGCCAUAAUCUUGUG | XXOOOOOOOOOOOOOOOOOO |
| | UmGmUmGmCmU*mU*mU | CUUU | XX |
| WV-42385 | mU*fU*mAmCmAfGmGmCmCmAmUmAmAfUmCf | UUACAGGCCAUAAUCUUGU | XXOOOOOOOOOOOOOOOOOO |
| | UmUmGmUmGmC*mU*mU | GCUU | XX |
| WV-42386 | mU*fC*mCmAmAfUmAmCmAmGmGmCmCfAmUfA | UCCAAUACAGGCCAUAAUC | XXOOOOOOOOOOOOOOOOOO |
| | mAmUmCmUmU*mU*mU | UUUU | XX |
| WV-42387 | mU*fC*mUmUmCfUmUmAmCmGmAmCmUfUmCf | UCUUCUUACGACUUCAUCU | XXOOOOOOOOOOOOOOOOOO |
| | AmUmCmUmGmU*mU*mU | GUUU | XX |
| WV-42388 | mU*fU*mCmCmAfUmCmUmAmUmCmAmGfAmCf | UUCCAUCUAUCAGACUUCU | XXOOOOOOOOOOOOOOOOOO |
| | UmUmCmUmUmA*mU*mU | UAUU | XX |
| WV-42389 | mU*fA*mGmUmAfUmUmCmCmAmUmCmUfAmUf | UAGUAUUCCAUCUAUCAGA | XXOOOOOOOOOOOOOOOOOO |
| | CmAmGmAmCmU*mU*mU | CUUU | XX |
| WV-42390 | mU*fA*mAmGmUfAmUmUmCmCmAmUmCfUmAf | UAAGUAUUCCAUCUAUCAG | XXOOOOOOOOOOOOOOOOOO |
| | UmCmAmGmAmC*mU*mU | ACUU | XX |
| WV-42391 | mU*fA*mUmUmGfGmUmAmAmGmUmAmUfUmCf | UAUUGGUAAGUAUUCCAU | XXOOOOOOOOOOOOOOOOOO |
| | CmAmUmCmUmA*mU*mU | CUAUU | XX |
| WV-42392 | mU*fU*mAmUmUfGmGmUmAmAmGmUmAfUmUf | UUAUUGGUAAGUAUUCCA | XXOOOOOOOOOOOOOOOOOO |
| | CmCmAmUmCmU*mU*mU | UCUUU | XX |
| WV-42393 | mU*fC*mUmUmAfUmUmGmGmUmAmAmGfUmAf | UCUUAUUGGUAAGUAUUC | XXOOOOOOOOOOOOOOOOOO |
| | UmUmCmCmAmU*mU*mU | CAUUU | XX |
| WV-42394 | mU*fU*mCmUmUfAmUmUmGmGmUmAmAfGmUf | UUCUUAUUGGUAAGUAUU | XXOOOOOOOOOOOOOOOOOO |
| | AmUmUmCmCmA*mU*mU | CCAUU | XX |
| WV-42395 | mU*fA*mAmAmAfUmCmAmUmUmUmUmCfUmUf | UAAAAUCAUUUUCUUAUU | XXOOOOOOOOOOOOOOOOOO |
| | AmUmUmGmGmU*mU*mU | GGUUU | XX |
| WV-42396 | mU*fA*mGmAmGfUmUmGmCmAmCmCmGfUmUf | UAGAGUUGCACCGUUUUG | XXOOOOOOOOOOOOOOOOOO |
| | UmUmGmGmGmC*mU*mU | GGCUU | XX |
| WV-42397 | mU*fA*mGmUmCfCmAmGmAmAmUmAmGfAmGf | UAGUCCAGAAUAGAGUUGC | XXOOOOOOOOOOOOOOOOOO |
| | UmUmGmCmAmC*mU*mU | ACUU | XX |
| WV-42398 | Mod001L001mA*mGmAmGmCmAfAmAfGfCfCmA | AGAGCAAAGCCAUGAACAU | OXOOOOOOOOOOOOOOOOOO |
| | mUmGmAmAmCmAmUmC*mA | CA | X |
| WV-42399 | Mod001L001mA*mAmAmGmCmCfAmUfGfAfAmC | AAAGCCAUGAACAUCAUCC | OXOOOOOOOOOOOOOOOOOO |
| | mAmUmCmAmUmCmCmU*mA | UA | X |
| WV-42400 | Mod001L001mC*mAmUmGmAmAfCmAfUfCfAmU | CAUGAACAUCAUCCUAGAA | OXOOOOOOOOOOOOOOOOOO |
| | mCmCmUmAmGmAmAmA*mA | AA | X |
| WV-42401 | Mod001L001mA*mAmCmAmUmCfAmUfCfCfUmA | AACAUCAUCCUAGAAAUCC | OXOOOOOOOOOOOOOOOOOO |
| | mGmAmAmAmUmCmCmU*mA | UA | X |
| WV-42402 | Mod001L001mA*mUmCmCmUmAfGmAfAfAfUmC | AUCCUAGAAAUCCUUCUGC | OXOOOOOOOOOOOOOOOOOO |
| | mCmUmUmCmUmGmCmU*mA | UA | X |
| WV-42403 | Mod001L001mC*mCmUmAmGmAfAmAfUfCfCmU | CCUAGAAAUCCUUCUGCUU | OXOOOOOOOOOOOOOOOOOO |
| | mUmCmUmGmCmUmUmC*mA | CA | X |
| WV-42404 | Mod001L001mC*mUmGmCmUmUfCmUfGfAfUmC | CUGCUUCUGAUCACCAUCA | OXOOOOOOOOOOOOOOOOOO |
| | mAmCmCmAmUmCmAmU*mA | UA | X |
| WV-42405 | Mod001L001mG*mCmUmUmCmUfGmAfUfCfAmC | GCUUCUGAUCACCAUCAUC | OXOOOOOOOOOOOOOOOOOO |
| | mCmAmUmCmAmUmCmU*mA | UA | X |
| WV-42406 | Mod001L001mG*mGmGmGmAmGfAmUfUfGfUmU | GGGGAGAUUGUUCUCAUU | OXOOOOOOOOOOOOOOOOOO |
| | mCmUmCmAmUmUmAmC*mA | ACA | X |
| WV-42407 | Mod001L001mG*mGmGmAmGmAfUmUfGfUfUmC | GGGAGAUUGUUCUCAUUA | OXOOOOOOOOOOOOOOOOOO |
| | mUmCmAmUmUmAmCmU*mA | CUA | X |
| WV-42408 | Mod001L001mA*mAmAmGmAmAfGmUfGfGfGmU | AAAGAAGUGGGUGAUGUA | OXOOOOOOOOOOOOOOOOOO |
| | mGmAmUmGmUmAmAmC*mA | ACA | X |
| WV-42409 | Mod001L001mG*mUmGmAmUmGfUmAfAfCfAmA | GUGAUGUAACAAUCGUGG | OXOOOOOOOOOOOOOOOOOO |
| | mUmCmGmUmGmGmUmG*mA | UGA | X |
| WV-42410 | Mod001L001mU*mGmUmAmAmCfAmAfUfCfGmU | UGUAACAAUCGUGGUGAA | OXOOOOOOOOOOOOOOOOOO |
| | mGmGmUmGmAmAmUmA*mA | UAA | X |
| WV-42411 | Mod001L001mA*mCmAmGmUmAfUmAfUfCfCmA | ACAGUAUAUCCAGCCGAUC | OXOOOOOOOOOOOOOOOOOO |
| | mGmCmCmGmAmUmCmU*mA | UA | X |
| WV-42412 | Mod001L001mC*mAmGmCmAmCfCmAfAfGfGmAm | CAGCACCAAGGAUGAAGAG | OXOOOOOOOOOOOOOOOOOO |
| | UmGmAmAmGmAmGmA*mA | AA | X |
| WV-42413 | Mod001L001mA*mGmCmAmCmCfAmAfGfGfAmU | AGCACCAAGGAUGAAGAGA | OXOOOOOOOOOOOOOOOOOO |
| | mGmAmAmGmAmGmAmU*mA | UA | X |
| WV-42414 | Mod001L001mC*mCmAmAmGmGfAmUfGfAfAmG | CCAAGGAUGAAGAGAUUAC | OXOOOOOOOOOOOOOOOOOO |
| | mAmGmAmUmUmAmCmC*mA | CA | X |
| WV-42415 | Mod001L001mG*mAmCmAmUmUfUmGfAfGfGmU | GACAUUUGAGGUCAACAUC | OXOOOOOOOOOOOOOOOOOO |
| | mCmAmAmCmAmUmCmC*mA | CA | X |
| WV-42416 | Mod001L001mU*mCmCmUmAmGfGmAfCfAfUmU | UCCUAGGACAUUUUUGGA | OXOOOOOOOOOOOOOOOOOO |
| | mUmUmUmGmGmAmUmC*mA | UCA | X |
| WV-42417 | Mod001L001mC*mUmAmGmGmAfCmAfUfUfUmU | CUAGGACAUUUUUGGAUC | OXOOOOOOOOOOOOOOOOOO |
| | mUmGmGmAmUmCmAmC*mA | ACA | X |
| WV-42418 | Mod001L001mU*mUmGmGmAmUfCmAfCfAfAmA | UUGGAUCACAAAAGCACUU | OXOOOOOOOOOOOOOOOOOO |
| | mAmGmCmAmCmUmUmC*mA | CA | X |
| WV-42419 | Mod001L001mU*mCmAmCmAmAfAmAfGfCfAmCm | UCACAAAAGCACUUCUUCC | OXOOOOOOOOOOOOOOOOOO |
| | UmUmCmUmUmCmCmA*mA | AA | X |
| WV-42420 | Mod001L001mA+mUmCmCmCmAfUmAfUfUfGmU | AUCCCAUAUUGUUCCAGCA | OXOOOOOOOOOOOOOOOOOO |
| | mUmCmCmAmGmCmAmA*mA | AA | X |
| WV-42421 | Mod001L001mG*mCmCmGmCmUfGmUfUfGfGmC | GCCGCUGUUGGCUUUCACA | OXOOOOOOOOOOOOOOOOOO |
| | mUmUmUmCmAmCmAmG*mA | GA | X |
| WV-42422 | Mod001L001mU*mUmGmGmCmUfUmUfCfAfCmA | UUGGCUUUCACAGAGGUC | OXOOOOOOOOOOOOOOOOOO |
| | mGmAmGmGmUmCmUmG*mA | UGA | X |
| WV-42423 | Mod001L001mG*mGmCmUmUmUfCmAfCfAfGmA | GGCUUUCACAGAGGUCUGA | OXOOOOOOOOOOOOOOOOOO |
| | mGmGmUmCmUmGmAmC*mA | CA | X |
| WV-42424 | Mod001L001mA*mGmGmUmCmUfGmAfCfAfUmC | AGGUCUGACAUCAGAACUU | OXOOOOOOOOOOOOOOOOOO |
| | mAmGmAmAmCmUmUmC*mA | CA | X |
| WV-42425 | Mod001L001mU*mUmGmGmGmAfAmAfAfAfCmU | UUGGGAAAAACUGGUAUC | OXOOOOOOOOOOOOOOOOOO |
| | mGmGmUmAmUmCmAmA*mA | AAA | X |
| WV-42426 | Mod001L001mU*mGmGmGmAmAfAmAfAfCfUmG | UGGGAAAAACUGGUAUCAA | OXOOOOOOOOOOOOOOOOOO |
| | mGmUmAmUmCmAmAmA*mA | AA | X |
| WV-42427 | Mod001L001mU*mGmGmUmAmUfCmAfAfAfAmC | UGGUAUCAAAACCUCAUGU | OXOOOOOOOOOOOOOOOOOO |
| | mCmUmCmAmUmGmUmC*mA | CA | X |
| WV-42428 | Mod001L001mA*mGmCmAmCmAfAmGfAfUfUmA | AGCACAAGAUUAUGGCCUG | OXOOOOOOOOOOOOOOOOOO |
| | mUmGmGmCmCmUmGmU*mA | UA | X |
| WV-42429 | Mod001L001mG*mCmAmCmAmAfGmAfUfUfAmU | GCACAAGAUUAUGGCCUGU | OXOOOOOOOOOOOOOOOOOO |
| | mGmGmCmCmUmGmUmA*mA | AA | X |
| WV-42430 | Mod001L001mA*mAmGmAmUmUfAmUfGfGfCmC | AAGAUUAUGGCCUGUAUU | OXOOOOOOOOOOOOOOOOOO |
| | mUmGmUmAmUmUmGmG*mA | GGA | X |
| WV-42431 | Mod001L001mA*mCmAmGmAmUfGmAfAfGfUmC | ACAGAUGAAGUCGUAAGAA | OXOOOOOOOOOOOOOOOOOO |
| | mGmUmAmAmGmAmAmG*mA | GA | X |
| WV-42432 | Mod001L001mU*mAmAmGmAmAfGmUfCfUfGmA | UAAGAAGUCUGAUAGAUG | OXOOOOOOOOOOOOOOOOOO |
| | mUmAmGmAmUmGmGmA*mA | GAA | X |
| WV-42433 | Mod001L001mA*mGmUmCmUmGfAmUfAfGfAmU | AGUCUGAUAGAUGGAAUA | OXOOOOOOOOOOOOOOOOOO |
| | mGmGmAmAmUmAmCmU*mA | CUA | X |
| WV-42434 | Mod001L001mG*mUmCmUmGmAfUmAfGfAfUmG | GUCUGAUAGAUGGAAUAC | OXOOOOOOOOOOOOOOOOOO |
| | mGmAmAmUmAmCmUmU*mA | UUA | X |
| WV-42435 | Mod001L001mU*mAmGmAmUmGfGmAfAfUfAmC | UAGAUGGAAUACUUACCAA | OXOOOOOOOOOOOOOOOOOO |
| | mUmUmAmCmCmAmAmU*mA | UA | X |
| WV-42436 | Mod001L001mA*mGmAmUmGmGfAmAfUfAfCmU | AGAUGGAAUACUUACCAAU | OXOOOOOOOOOOOOOOOOOO |
| | mUmAmCmCmAmAmUmA*mA | AA | X |
| WV-42437 | Mod001L001mA*mUmGmGmAmAfUmAfCfUfUmA | AUGGAAUACUUACCAAUAA | OXOOOOOOOOOOOOOOOOOO |
| | mCmCmAmAmUmAmAmG*mA | GA | X |
| WV-42438 | Mod001L001mU*mGmGmAmAmUfAmCfUfUfAmC | UGGAAUACUUACCAAUAAG | OXOOOOOOOOOOOOOOOOOO |
| | mCmAmAmUmAmAmGmA*mA | AA | X |
| WV-42439 | Mod001L001mA*mCmCmAmAmUfAmAfGfAfAmA | ACCAAUAAGAAAAUGAUUU | OXOOOOOOOOOOOOOOOOOO |
| | mAmUmGmAmUmUmUmU*mA | UA | X |
| WV-42440 | Mod001L001mG*mCmCmCmAmAfAmAfCfGfGmU | GCCCAAAACGGUGCAACUC | OXOOOOOOOOOOOOOOOOOO |
| | mGmCmAmAmCmUmCmU*mA | UA | X |
| WV-42441 | Mod001L001mG*mUmGmCmAmAfCmUfCfUfAmU | GUGCAACUCUAUUCUGGAC | OXOOOOOOOOOOOOOOOOOO |
| | mUmCmUmGmGmAmCmU*mA | UA | X |
| WV-42442 | L001mA*mGmAmGmCmAfAmAfGfCfCmAmUmGm | AGAGCAAAGCCAUGAACAU | OXOOOOOOOOOOOOOOOOOO |
| | AmAmCmAmUmC*mA | CA | X |
| WV-42443 | L001mA*mAmAmGmCmCfAmUfGfAfAmCmAmUmC | AAAGCCAUGAACAUCAUCC | OXOOOOOOOOOOOOOOOOOO |
| | mAmUmCmCmU*mA | UA | X |
| WV-42444 | L001mC*mAmUmGmAmAfCmAfUfCfAmUmCmCmU | CAUGAACAUCAUCCUAGAA | OXOOOOOOOOOOOOOOOOOO |
| | mAmGmAmAmA*mA | AA | X |
| WV-42445 | L001mA*mAmCmAmUmCfAmUfCfCfUmAmGmAmA | AACAUCAUCCUAGAAAUCC | OXOOOOOOOOOOOOOOOOOO |
| | mAmUmCmCmU*mA | UA | X |
| WV-42446 | L001mA*mUmCmCmUmAfGmAfAfAfUmCmCmUm | AUCCUAGAAAUCCUUCUGC | OXOOOOOOOOOOOOOOOOOO |
| | UmCmUmGmCmU*mA | UA | X |
| WV-42447 | L001mC*mCmUmAmGmAfAmAfUfCfCmUmUmCm | CCUAGAAAUCCUUCUGCUU | OXOOOOOOOOOOOOOOOOOO |
| | UmGmCmUmUmC*mA | CA | X |
| WV-42448 | L001mC*mUmGmCmUmUfCmUfGfAfUmCmAmCm | CUGCUUCUGAUCACCAUCA | OXOOOOOOOOOOOOOOOOOO |
| | CmAmUmCmAmU*mA | UA | X |
| WV-42449 | L001mG*mCmUmUmCmUfGmAfUfCfAmCmCmAm | GCUUCUGAUCACCAUCAUC | OXOOOOOOOOOOOOOOOOOO |
| | UmCmAmUmCmU*mA | UA | X |
| WV-42450 | L001mG*mGmGmGmAmGfAmUfUfGfUmUmCmUm | GGGGAGAUUGUUCUCAUU | OXOOOOOOOOOOOOOOOOOO |
| | CmAmUmUmAmC*mA | ACA | X |
| WV-42451 | L001mG*mGmGmAmGmAfUmUfGfUfUmCmUmCm | GGGAGAUUGUUCUCAUUA | OXOOOOOOOOOOOOOOOOOO |
| | AmUmUmAmCmU*mA | CUA | X |
| WV-42452 | L001mA*mAmAmGmAmAfGmUfGfGfGmUmGmAm | AAAGAAGUGGGUGAUGUA | OXOOOOOOOOOOOOOOOOOO |
| | UmGmUmAmAmC*mA | ACA | X |
| WV-42453 | L001mG*mUmGmAmUmGfUmAfAfCfAmAmUmCm | GUGAUGUAACAAUCGUGG | OXOOOOOOOOOOOOOOOOOO |
| | GmUmGmGmUmG*mA | UGA | X |
| WV-42454 | L001mU*mGmUmAmAmCfAmAfUfCfGmUmGmGm | UGUAACAAUCGUGGUGAA | OXOOOOOOOOOOOOOOOOOO |
| | UmGmAmAmUmA*mA | UAA | X |
| WV-42455 | L001mA*mCmAmGmUmAfUmAfUfCfCmAmGmCmC | ACAGUAUAUCCAGCCGAUC | OXOOOOOOOOOOOOOOOOOO |
| | mGmAmUmCmU*mA | UA | X |
| WV-42456 | L001mC*mAmGmCmAmCfCmAfAfGfGmAmUmGmA | CAGCACCAAGGAUGAAGAG | OXOOOOOOOOOOOOOOOOOO |
| | mAmGmAmGmA*mA | AA | X |
| WV-42457 | L001mA*mGmCmAmCmCfAmAfGfGfAmUmGmAm | AGCACCAAGGAUGAAGAGA | OXOOOOOOOOOOOOOOOOOO |
| | AmGmAmGmAmU*mA | UA | X |
| WV-42458 | L001mC*mCmAmAmGmGfAmUfGfAfAmGmAmGm | CCAAGGAUGAAGAGAUUAC | OXOOOOOOOOOOOOOOOOOO |
| | AmUmUmAmCmC*mA | CA | X |
| WV-42459 | L001mG*mAmCmAmUmUfUmGfAfGfGmUmCmAm | GACAUUUGAGGUCAACAUC | OXOOOOOOOOOOOOOOOOOO |
| | AmCmAmUmCmC*mA | CA | X |
| WV-42460 | L001mU*mCmCmUmAmGfGmAfCfAfUmUmUmUm | UCCUAGGACAUUUUUGGA | OXOOOOOOOOOOOOOOOOOO |
| | UmGmGmAmUmC*mA | UCA | X |
| WV-42461 | L001mC*mUmAmGmGmAfCmAfUfUfUmUmUmGm | CUAGGACAUUUUUGGAUC | OXOOOOOOOOOOOOOOOOOO |
| | GmAmUmCmAmC*mA | ACA | X |
| WV-42462 | L001mU*mUmGmGmAmUfCmAfCfAfAmAmAmGm | UUGGAUCACAAAAGCACUU | OXOOOOOOOOOOOOOOOOOO |
| | CmAmCmUmUmC*mA | CA | X |
| WV-42463 | L001mU*mCmAmCmAmAfAmAfGfCfAmCmUmUmC | UCACAAAAGCACUUCUUCC | OXOOOOOOOOOOOOOOOOOO |
| | mUmUmCmCmA*mA | AA | X |
| WV-42464 | L001mA*mUmCmCmCmAfUmAfUfUfGmUmUmCm | AUCCCAUAUUGUUCCAGCA | OXOOOOOOOOOOOOOOOOOO |
| | CmAmGmCmAmA*mA | AA | X |
| WV-42465 | L001mG*mCmCmGmCmUfGmUfUfGfGmCmUmUm | GCCGCUGUUGGCUUUCACA | OXOOOOOOOOOOOOOOOOOO |
| | UmCmAmCmAmG*mA | GA | X |
| WV-42466 | L001mU*mUmGmGmCmUfUmUfCfAfCmAmGmAm | UUGGCUUUCACAGAGGUC | OXOOOOOOOOOOOOOOOOOO |
| | GmGmUmCmUmG*mA | UGA | X |
| WV-42467 | L001mG*mGmCmUmUmUfCmAfCfAfGmAmGmGm | GGCUUUCACAGAGGUCUGA | OXOOOOOOOOOOOOOOOOOO |
| | UmCmUmGmAmC*mA | CA | X |
| WV-42468 | L001mA*mGmGmUmCmUfGmAfCfAfUmCmAmGm | AGGUCUGACAUCAGAACUU | OXOOOOOOOOOOOOOOOOOO |
| | AmAmCmUmUmC*mA | CA | X |
| WV-42469 | L001mU*mUmGmGmGmAfAmAfAfAfCmUmGmGm | UUGGGAAAAACUGGUAUC | OXOOOOOOOOOOOOOOOOOO |
| | UmAmUmCmAmA*mA | AAA | X |
| WV-42470 | L001mU*mGmGmGmAmAfAmAfAfCfUmGmGmUm | UGGGAAAAACUGGUAUCAA | OXOOOOOOOOOOOOOOOOOO |
| | AmUmCmAmAmA*mA | AA | X |
| WV-42471 | L001mU*mGmGmUmAmUfCmAfAfAfAmCmCmUm | UGGUAUCAAAACCUCAUGU | OXOOOOOOOOOOOOOOOOOO |
| | CmAmUmGmUmC*mA | CA | X |
| WV-42472 | L001mA*mGmCmAmCmAfAmGfAfUfUmAmUmGm | AGCACAAGAUUAUGGCCUG | OXOOOOOOOOOOOOOOOOOO |
| | GmCmCmUmGmU*mA | UA | X |
| WV-42473 | L001mG*mCmAmCmAmAfGmAfUfUfAmUmGmGm | GCACAAGAUUAUGGCCUGU | OXOOOOOOOOOOOOOOOOOO |
| | CmCmUmGmUmA*mA | AA | X |
| WV-42474 | L001mA*mAmGmAmUmUfAmUfGfGfCmCmUmGm | AAGAUUAUGGCCUGUAUU | OXOOOOOOOOOOOOOOOOOO |
| | UmAmUmUmGmG*mA | GGA | X |
| WV-42475 | L001mA*mCmAmGmAmUfGmAfAfGfUmCmGmUm | ACAGAUGAAGUCGUAAGAA | OXOOOOOOOOOOOOOOOOOO |
| | AmAmGmAmAmG*mA | GA | X |
| WV-42476 | L001mU*mAmAmGmAmAfGmUfCfUfGmAmUmAm | UAAGAAGUCUGAUAGAUG | OXOOOOOOOOOOOOOOOOOO |
| | GmAmUmGmGmA*mA | GAA | X |
| WV-42477 | L001mA*mGmUmCmUmGfAmUfAfGfAmUmGmGm | AGUCUGAUAGAUGGAAUA | OXOOOOOOOOOOOOOOOOOO |
| | AmAmUmAmCmU*mA | CUA | X |
| WV-42478 | L001mG*mUmCmUmGmAfUmAfGfAfUmGmGmAm | GUCUGAUAGAUGGAAUAC | OXOOOOOOOOOOOOOOOOOO |
| | AmUmAmCmUmU*mA | UUA | X |
| WV-42479 | L001mU*mAmGmAmUmGfGmAfAfUfAmCmUmUm | UAGAUGGAAUACUUACCAA | OXOOOOOOOOOOOOOOOOOO |
| | AmCmCmAmAmU*mA | UA | X |
| WV-42480 | L001mA*mGmAmUmGmGfAmAfUfAfCmUmUmAm | AGAUGGAAUACUUACCAAU | OXOOOOOOOOOOOOOOOOOO |
| | CmCmAmAmUmA*mA | AA | X |
| WV-42481 | L001mA*mUmGmGmAmAfUmAfCfUfUmAmCmCm | AUGGAAUACUUACCAAUAA | OXOOOOOOOOOOOOOOOOOO |
| | AmAmUmAmAmG*mA | GA | X |
| WV-42482 | L001mU*mGmGmAmAmUfAmCfUfUfAmCmCmAm | UGGAAUACUUACCAAUAAG | OXOOOOOOOOOOOOOOOOOO |
| | AmUmAmAmGmA*mA | AA | X |
| WV-42483 | L001mA*mCmCmAmAmUfAmAfGfAfAmAmAmUm | ACCAAUAAGAAAAUGAUUU | OXOOOOOOOOOOOOOOOOOO |
| | GmAmUmUmUmU*mA | UA | X |
| WV-42484 | L001mG*mCmCmCmAmAfAmAfCfGfGmUmGmCmA | GCCCAAAACGGUGCAACUC | OXOOOOOOOOOOOOOOOOOO |
| | mAmCmUmCmU*mA | UA | X |
| WV-42485 | L001mG*mUmGmCmAmAfCmUfCfUfAmUmUmCm | GUGCAACUCUAUUCUGGAC | OXOOOOOOOOOOOOOOOOOO |
| | UmGmGmAmCmU*mA | UA | X |
| WV-42531 | L025L025mA*mGmAmGmCmAfAmAfGfCfCmAmUm | AGAGCAAAGCCAUGAACAU | OOXOOOOOOOOOOOOOOOOO |
| | GmAmAmCmAmUmC*mA | CA | OX |
| WV-42532 | L025L025mA*mAmAmGmCmCfAmUfGfAfAmCmAm | AAAGCCAUGAACAUCAUCC | OOXOOOOOOOOOOOOOOOOO |
| | UmCmAmUmCmCmU*mA | UA | OX |
| WV-42533 | L025L025mC*mAmUmGmAmAfCmAfUfCfAmUmCm | CAUGAACAUCAUCCUAGAA | OOXOOOOOOOOOOOOOOOOO |
| | CmUmAmGmAmAmA*mA | AA | OX |
| WV-42534 | L025L025mA*mAmCmAmUmCfAmUfCfCfUmAmGm | AACAUCAUCCUAGAAAUCC | OOXOOOOOOOOOOOOOOOOO |
| | AmAmAmUmCmCmU*mA | UA | OX |
| WV-42535 | L025L025mA*mUmCmCmUmAfGmAfAfAfUmCmCm | AUCCUAGAAAUCCUUCUGC | OOXOOOOOOOOOOOOOOOOO |
| | UmUmCmUmGmCmU*mA | UA | OX |
| WV-42536 | L025L025mC*mCmUmAmGmAfAmAfUfCfCmUmU | CCUAGAAAUCCUUCUGCUU | OOXOOOOOOOOOOOOOOOOO |
| | mCmUmGmCmUmUmC*mA | CA | OX |
| WV-42537 | L025L025mC*mUmGmCmUmUfCmUfGfAfUmCmA | CUGCUUCUGAUCACCAUCA | OOXOOOOOOOOOOOOOOOOO |
| | mCmCmAmUmCmAmU*mA | UA | OX |
| WV-42538 | L025L025mG*mCmUmUmCmUfGmAfUfCfAmCmC | GCUUCUGAUCACCAUCAUC | OOXOOOOOOOOOOOOOOOOO |
| | mAmUmCmAmUmCmU*mA | UA | OX |
| WV-42539 | L025L025mG*mGmGmGmAmGfAmUfUfGfUmUmC | GGGGAGAUUGUUCUCAUU | OOXOOOOOOOOOOOOOOOOO |
| | mUmCmAmUmUmAmC*mA | ACA | OX |
| WV-42540 | L025L025mG*mGmGmAmGmAfUmUfGfUfUmCmU | GGGAGAUUGUUCUCAUUA | OOXOOOOOOOOOOOOOOOOO |
| | mCmAmUmUmAmCmU*mA | CUA | OX |
| WV-42541 | L025L025mA*mAmAmGmAmAfGmUfGfGfGmUmG | AAAGAAGUGGGUGAUGUA | OOXOOOOOOOOOOOOOOOOO |
| | mAmUmGmUmAmAmC*mA | ACA | OX |
| WV-42542 | L025L025mG*mUmGmAmUmGfUmAfAfCfAmAmU | GUGAUGUAACAAUCGUGG | OOXOOOOOOOOOOOOOOOOO |
| | mCmGmUmGmGmUmG*mA | UGA | OX |
| WV-42543 | L025L025mU*mGmUmAmAmCfAmAfUfCfGmUmG | UGUAACAAUCGUGGUGAA | OOXOOOOOOOOOOOOOOOOO |
| | mGmUmGmAmAmUmA*mA | UAA | OX |
| WV-42544 | L025L025mA*mCmAmGmUmAfUmAfUfCfCmAmG | ACAGUAUAUCCAGCCGAUC | OOXOOOOOOOOOOOOOOOOO |
| | mCmCmGmAmUmCmU*mA | UA | OX |
| WV-42545 | L025L025mC*mAmGmCmAmCfCmAfAfGfGmAmUm | CAGCACCAAGGAUGAAGAG | OOXOOOOOOOOOOOOOOOOO |
| | GmAmAmGmAmGmA*mA | AA | OX |
| WV-42546 | L025L025mA*mGmCmAmCmCfAmAfGfGfAmUmG | AGCACCAAGGAUGAAGAGA | OOXOOOOOOOOOOOOOOOOO |
| | mAmAmGmAmGmAmU*mA | UA | OX |
| WV-42547 | L025L025mC*mCmAmAmGmGfAmUfGfAfAmGmA | CCAAGGAUGAAGAGAUUAC | OOXOOOOOOOOOOOOOOOOO |
| | mGmAmUmUmAmCmC*mA | CA | OX |
| WV-42548 | L025L025mG*mAmCmAmUmUfUmGfAfGfGmUmC | GACAUUUGAGGUCAACAUC | OOXOOOOOOOOOOOOOOOOO |
| | mAmAmCmAmUmCmC*mA | CA | OX |
| WV-42549 | L025L025mU*mCmCmUmAmGfGmAfCfAfUmUmU | UCCUAGGACAUUUUUGGA | OOXOOOOOOOOOOOOOOOOO |
| | mUmUmGmGmAmUmC*mA | UCA | OX |
| WV-42550 | L025L025mC*mUmAmGmGmAfCmAfUfUfUmUmU | CUAGGACAUUUUUGGAUC | OOXOOOOOOOOOOOOOOOOO |
| | mGmGmAmUmCmAmC*mA | ACA | OX |
| WV-42551 | L025L025mU*mUmGmGmAmUfCmAfCfAfAmAmA | UUGGAUCACAAAAGCACUU | OOXOOOOOOOOOOOOOOOOO |
| | mGmCmAmCmUmUmC*mA | CA | OX |
| WV-42552 | L025L025mU*mCmAmCmAmAfAmAfGfCfAmCmUm | UCACAAAAGCACUUCUUCC | OOXOOOOOOOOOOOOOOOOO |
| | UmCmUmUmCmCmA*mA | AA | OX |
| WV-42553 | L025L025mA*mUmCmCmCmAfUmAfUfUfGmUmU | AUCCCAUAUUGUUCCAGCA | OOXOOOOOOOOOOOOOOOOO |
| | mCmCmAmGmCmAmA*mA | AA | OX |
| WV-42554 | L025L025mG*mCmCmGmCmUfGmUfUfGfGmCmU | GCCGCUGUUGGCUUUCACA | OOXOOOOOOOOOOOOOOOOO |
| | mUmUmCmAmCmAmG*mA | GA | OX |
| WV-42555 | L025L025mU*mUmGmGmCmUfUmUfCfAfCmAmG | UUGGCUUUCACAGAGGUC | OOXOOOOOOOOOOOOOOOOO |
| | mAmGmGmUmCmUmG*mA | UGA | OX |
| WV-42556 | L025L025mG*mGmCmUmUmUfCmAfCfAfGmAmG | GGCUUUCACAGAGGUCUGA | OOXOOOOOOOOOOOOOOOOO |
| | mGmUmCmUmGmAmC*mA | CA | OX |
| WV-42557 | L025L025mA*mGmGmUmCmUfGmAfCfAfUmCmA | AGGUCUGACAUCAGAACUU | OOXOOOOOOOOOOOOOOOOO |
| | mGmAmAmCmUmUmC*mA | CA | OX |
| WV-42558 | L025L025mU*mUmGmGmGmAfAmAfAfAfCmUmG | UUGGGAAAAACUGGUAUC | OOXOOOOOOOOOOOOOOOOO |
| | mGmUmAmUmCmAmA*mA | AAA | OX |
| WV-42559 | L025L025mU*mGmGmGmAmAfAmAfAfCfUmGmG | UGGGAAAAACUGGUAUCAA | OOXOOOOOOOOOOOOOOOOO |
| | mUmAmUmCmAmAmA*mA | AA | OX |
| WV-42560 | L025L025mU*mGmGmUmAmUfCmAfAfAfAmCmC | UGGUAUCAAAACCUCAUGU | OOXOOOOOOOOOOOOOOOOO |
| | mUmCmAmUmGmUmC*mA | CA | OX |
| WV-42561 | L025L025mA*mGmCmAmCmAfAmGfAfUfUmAmU | AGCACAAGAUUAUGGCCUG | OOXOOOOOOOOOOOOOOOOO |
| | mGmGmCmCmUmGmU*mA | UA | OX |
| WV-42562 | L025L025mG*mCmAmCmAmAfGmAfUfUfAmUmG | GCACAAGAUUAUGGCCUGU | OOXOOOOOOOOOOOOOOOOO |
| | mGmCmCmUmGmUmA*mA | AA | OX |
| WV-42563 | L025L025mA*mAmGmAmUmUfAmUfGfGfCmCmU | AAGAUUAUGGCCUGUAUU | OOXOOOOOOOOOOOOOOOOO |
| | mGmUmAmUmUmGmG*mA | GGA | OX |
| WV-42564 | L025L025mA*mCmAmGmAmUfGmAfAfGfUmCmG | ACAGAUGAAGUCGUAAGAA | OOXOOOOOOOOOOOOOOOOO |
| | mUmAmAmGmAmAmG*mA | GA | OX |
| WV-42565 | L025L025mU*mAmAmGmAmAfGmUfCfUfGmAmU | UAAGAAGUCUGAUAGAUG | OOXOOOOOOOOOOOOOOOOO |
| | mAmGmAmUmGmGmA*mA | GAA | OX |
| WV-42566 | L025L025mA*mGmUmCmUmGfAmUfAfGfAmUmG | AGUCUGAUAGAUGGAAUA | OOXOOOOOOOOOOOOOOOOO |
| | mGmAmAmUmAmCmU*mA | CUA | OX |
| WV-42567 | L025L025mG*mUmCmUmGmAfUmAfGfAfUmGmG | GUCUGAUAGAUGGAAUAC | OOXOOOOOOOOOOOOOOOOO |
| | mAmAmUmAmCmUmU*mA | UUA | OX |
| WV-42568 | L025L025mU*mAmGmAmUmGfGmAfAfUfAmCmU | UAGAUGGAAUACUUACCAA | OOXOOOOOOOOOOOOOOOOO |
| | mUmAmCmCmAmAmU*mA | UA | OX |
| WV-42569 | L025L025mA*mGmAmUmGmGfAmAfUfAfCmUmU | AGAUGGAAUACUUACCAAU | OOXOOOOOOOOOOOOOOOOO |
| | mAmCmCmAmAmUmA*mA | AA | OX |
| WV-42570 | L025L025mA*mUmGmGmAmAfUmAfCfUfUmAmC | AUGGAAUACUUACCAAUAA | OOXOOOOOOOOOOOOOOOOO |
| | mCmAmAmUmAmAmG*mA | GA | OX |
| WV-42571 | L025L025mU*mGmGmAmAmUfAmCfUfUfAmCmC | UGGAAUACUUACCAAUAAG | OOXOOOOOOOOOOOOOOOOO |
| | mAmAmUmAmAmGmA*mA | AA | OX |
| WV-42572 | L025L025mA*mCmCmAmAmUfAmAfGfAfAmAmAm | ACCAAUAAGAAAAUGAUUU | OOXOOOOOOOOOOOOOOOOO |
| | UmGmAmUmUmUmU*mA | UA | OX |
| WV-42573 | L025L025mG*mCmCmCmAmAfAmAfCfGfGmUmGm | GCCCAAAACGGUGCAACUC | OOXOOOOOOOOOOOOOOOOO |
| | CmAmAmCmUmCmU*mA | UA | OX |
| WV-42574 | L025L025mG*mUmGmCmAmAfCmUfCfUfAmUmU | GUGCAACUCUAUUCUGGAC | OOXOOOOOOOOOOOOOOOOO |
| | mCmUmGmGmAmCmU*mA | UA | OX |
| WV-42575 | mU*SfG*RmAn001SmUmGfUmUmCmAmUn001Sm | UGAUGUUCAUGGCUUUGC | SRnSOOOOOOnSOOOOOOOOnS |
| | GmGmCfUmUfUmGmCmUn001SmCmU*SmU*SmU | UCUUU | OSS |
| WV-42576 | mU*SfU*RmUn001SmUmCfUmAmGmGmAn001Sm | UUUUCUAGGAUGAUGUUC | SRnSOOOOOOnSOOOOOOOOnS |
| | UmGmAfUmGfUmUmCmAn001SmUmG*SmU*SmU | AUGUU | OSS |
| WV-42577 | mU*SfG*RmAn001SmAmGfCmAmGmAmAn001Sm | UGAAGCAGAAGGAUUUCU | SRnSOOOOOOnSOOOOOOOOnS |
| | GmGmAfUmUfUmCmUmAn001SmGmG*SmU*SmU | AGGUU | OSS |
| WV-42578 | mU*SfG*RmUn001SmAmAfUmGmAmGmAn001Sm | UGUAAUGAGAACAAUCUCC | SRnSOOOOOOnSOOOOOOOOnS |
| | AmCmAfAmUfCmUmCmCn001SmCmC*SmU*SmU | CCUU | OSS |
| WV-42579 | mU*SfG*RmUn001SmUmAfCmAmUmCmAn001Sm | UGUUACAUCACCCACUUCU | SRnSOOOOOOnSOOOOOOOOnS |
| | CmCmCfAmCfUmUmCmUn001SmUmU*SmU*SmU | UUUU | OSS |
| WV-42580 | mU*SfG*RmUn001SmGmAfUmCmCmAmAn001Sm | UGUGAUCCAAAAAUGUCCU | SRnSOOOOOOnSOOOOOOOOnS |
| | AmAmAfUmGfUmCmCmUn001SmAmG*SmU*SmU | AGUU | OSS |
| WV-42581 | mU*SfG*RmAn001SmCmAfUmGmAmGmGn001Sm | UGACAUGAGGUUUUGAUA | SRnSOOOOOOnSOOOOOOOOnS |
| | UmUmUfUmGfAmUmAmCn001SmCmA*SmU*SmU | CCAUU | OSS |
| WV-42582 | mU*SfU*RmCn001SmUmUfAmUmUmGmGn001Sm | UUCUUAUUGGUAAGUAUU | SRnSOOOOOOnSOOOOOOOOnS |
| | UmAmAfGmUfAmUmUmCn001SmCmA*SmU*SmU | CCAUU | OSS |
| WV-42583 | Mod001L001mA*SmGmAmGmCmAfAmAfGfCfCmA | AGAGCAAAGCCAUGAACAU | OSOOOOOOOOOOOOOOOOOO |
| | mUmGmAmAmCmAmUmC*SmA | CA | S |
| WV-42584 | Mod001L001mC*SmAmUmGmAmAfCmAfUfCfAmU | CAUGAACAUCAUCCUAGAA | OSOOOOOOOOOOOOOOOOOO |
| | mCmCmUmAmGmAmAmA*SmA | AA | S |
| WV-42585 | Mod001L001mC*SmCmUmAmGmAfAmAfUfCfCmU | CCUAGAAAUCCUUCUGCUU | OSOOOOOOOOOOOOOOOOOO |
| | mUmCmUmGmCmUmUmC*SmA | CA | S |
| WV-42586 | Mod001L001mG*SmGmGmGmAmGfAmUfUfGfUm | GGGGAGAUUGUUCUCAUU | OSOOOOOOOOOOOOOOOOOO |
| | UmCmUmCmAmUmUmAmC*SmA | ACA | S |
| WV-42587 | Mod001L001mA*SmAmAmGmAmAfGmUfGfGfGm | AAAGAAGUGGGUGAUGUA | OSOOOOOOOOOOOOOOOOOO |
| | UmGmAmUmGmUmAmAmC*SmA | ACA | S |
| WV-42588 | Mod001L001mC*SmUmAmGmGmAfCmAfUfUfUmU | CUAGGACAUUUUUGGAUC | OSOOOOOOOOOOOOOOOOOO |
| | mUmGmGmAmUmCmAmC*SmA | ACA | S |
| WV-42589 | Mod001L001mU*SmGmGmUmAmUfCmAfAfAfAmC | UGGUAUCAAAACCUCAUGU | OSOOOOOOOOOOOOOOOOOO |
| | mCmUmCmAmUmGmUmC*SmA | CA | S |
| WV-42590 | Mod001L001mU*SmGmGmAmAmUfAmCfUfUfAmC | UGGAAUACUUACCAAUAAG | OSOOOOOOOOOOOOOOOOOO |
| | mCmAmAmUmAmAmGmA*SmA | AA | S |
| WV-42591 | L001mA*SmGmAmGmCmAfAmAfGfCfCmAmUmGm | AGAGCAAAGCCAUGAACAU | OSOOOOOOOOOOOOOOOOOO |
| | AmAmCmAmUmC*SmA | CA | S |
| WV-42592 | L001mC*SmAmUmGmAmAfCmAfUfCfAmUmCmCm | CAUGAACAUCAUCCUAGAA | OSOOOOOOOOOOOOOOOOOO |
| | UmAmGmAmAmA*SmA | AA | S |
| WV-42593 | L001mC*SmCmUmAmGmAfAmAfUfCfCmUmUmCm | CCUAGAAAUCCUUCUGCUU | OSOOOOOOOOOOOOOOOOOO |
| | UmGmCmUmUmC*SmA | CA | S |
| WV-42594 | L001mG*SmGmGmGmAmGfAmUfUfGfUmUmCmU | GGGGAGAUUGUUCUCAUU | OSOOOOOOOOOOOOOOOOOO |
| | mCmAmUmUmAmC*SmA | ACA | S |
| WV-42595 | L001mA*SmAmAmGmAmAfGmUfGfGfGmUmGmA | AAAGAAGUGGGUGAUGUA | OSOOOOOOOOOOOOOOOOOO |
| | mUmGmUmAmAmC*SmA | ACA | S |
| WV-42596 | L001mC*SmUmAmGmGmAfCmAfUfUfUmUmUmG | CUAGGACAUUUUUGGAUC | OSOOOOOOOOOOOOOOOOOO |
| | mGmAmUmCmAmC*SmA | ACA | S |
| WV-42597 | L001mU*SmGmGmUmAmUfCmAfAfAfAmCmCmU | UGGUAUCAAAACCUCAUGU | OSOOOOOOOOOOOOOOOOOO |
| | mCmAmUmGmUmC*SmA | CA | S |
| WV-42598 | L001mU*SmGmGmAmAmUfAmCfUfUfAmCmCmA | UGGAAUACUUACCAAUAAG | OSOOOOOOOOOOOOOOOOOO |
| | mAmUmAmAmGmA*SmA | AA | S |
| WV-42902 | mU*SfG*RmUn001SmUmAfCmAmUmCmAn001Sm | UGUUACAUCACCCACUUCU | SRnSOOOOOOnSOOSSSSSSn |
| | CmCmC*SfA*SmC*SfU*SmU*SmC*SmUn001SmU* | UUUU | SSSS |
| | SmU*SmU*SmU | | |
| WV-42903 | mU*SfG*RmAn001SmCmAfUmGmAmGmGn001Sm | UGACAUGAGGUUUUGAUA | SRnSOOOOOOnSOOSSSSSSn |
| | UmUmU*SfU*SmG*SfA*SmU*SmA*SmCn001SmC* | CCAUU | SSSS |
| | SmA*SmU*SmU | | |
| WV-43130 | mU*fG*mUmUmGfCmUmGmCmAmGmUmCfUmAf | UGUUGCUGCAGUCUACCAC | XXOOOOOOOOOOOOOOOOOO |
| | CmCmAmCmAmU*mU*mU | AUUU | XX |
| WV-43131 | mU*fC*mUmGmUfUmGmCmUmGmCmAmGfUmCf | UCUGUUGCUGCAGUCUACC | XXOOOOOOOOOOOOOOOOOO |
| | UmAmCmCmAmC*mU*mU | ACUU | XX |
| WV-43132 | mU*fC*mAmAmAfAmAmUmGmUmCmCmUfAmGf | UCAAAAAUGUCCUAGGAUG | XXOOOOOOOOOOOOOOOOOO |
| | GmAmUmGmUmU*mU*mU | UUUU | XX |
| WV-43133 | mU*fA*mAmUmAfUmUmCmUmGmCmAmUfAmCf | UAAUAUUCUGCAUACGAUU | XXOOOOOOOOOOOOOOOOOO |
| | GmAmUmUmUmA*mU*mU | UAUU | XX |
| WV-43134 | mU*fU*mUmGmAfAmUmAmUmUmCmUmGfCmAf | UUUGAAUAUUCUGCAUAC | XXOOOOOOOOOOOOOOOOOO |
| | UmAmCmGmAmU*mU*mU | GAUUU | XX |
| WV-43135 | mU*fA*mAmUmUfGmAmAmUmAmUmUmCfUmGf | UAAUUGAAUAUUCUGCAU | XXOOOOOOOOOOOOOOOOOO |
| | CmAmUmAmCmG*mU*mU | ACGUU | XX |
| WV-43136 | mU*fC*mAmAmAfUmUmGmAmAmUmAmUfUmCf | UCAAAUUGAAUAUUCUGCA | XXOOOOOOOOOOOOOOOOOO |
| | UmGmCmAmUmA*mU*mU | UAUU | XX |
| WV-43137 | mU*fA*mAmGmUfCmCmAmGmAmAmUmAfGmAf | UAAGUCCAGAAUAGAGUU | XXOOOOOOOOOOOOOOOOOO |
| | GmUmUmGmCmA*mU*mU | GCAUU | XX |
| WV-43138 | mU*fU*mAmAmAfGmUmCmCmAmGmAmAfUmAf | UUAAAGUCCAGAAUAGAGU | XXOOOOOOOOOOOOOOOOOO |
| | GmAmGmUmUmG*mU*mU | UGUU | XX |
| WV-43139 | mU*fC*mAmAmGfUmAmAmUmAmAmAmGfUmCf | UCAAGUAAUAAAGUCCAGA | XXOOOOOOOOOOOOOOOOOO |
| | CmAmGmAmAmU*mU*mU | AUUU | XX |
| WV-43140 | mU*fG*mAmGmGfGmUmCmCmAmCmUmUfUmUf | UGAGGGUCCACUUUUGGU | XXOOOOOOOOOOOOOOOOOO |
| | GmGmUmGmGmA*mU*mU | GGAUU | XX |
| WV-43141 | mU*fA*mGmAmGfGmGmUmCmCmAmCmUfUmUf | UAGAGGGUCCACUUUUGG | XXOOOOOOOOOOOOOOOOOO |
| | UmGmGmUmGmG*mU*mU | UGGUU | XX |
| WV-43142 | mU*fA*mUmAmGfAmGmGmGmUmCmCmAfCmUf | UAUAGAGGGUCCACUUUU | XXOOOOOOOOOOOOOOOOOO |
| | UmUmUmGmGmU*mU*mU | GGUUU | XX |
| WV-43143 | mU*fU*mAmUmAfGmAmGmGmGmUmCmCfAmCf | UUAUAGAGGGUCCACUUU | XXOOOOOOOOOOOOOOOOOO |
| | UmUmUmUmGmG*mU*mU | UGGUU | XX |
| WV-43144 | mU*fA*mAmGmGfGmAmGmGmAmAmAmUfAmUf | UAAGGGAGGAAAUAUAGA | XXOOOOOOOOOOOOOOOOOO |
| | AmGmAmGmGmG*mU*mU | GGGUU | XX |
| WV-43145 | mU*fU*mUmUmUfGmAmGmCmCmUmAmAfAmAf | UUUUUGAGCCUAAAAUUG | XXOOOOOOOOOOOOOOOOOO |
| | UmUmGmUmCmU*mU*mU | UCUUU | XX |
| WV-43146 | mU*fU*mAmAmUfAmGmCmCmAmGmUmAfCmAf | UUAAUAGCCAGUACAGUUC | XXOOOOOOOOOOOOOOOOOO |
| | GmUmUmCmCmU*mU*mU | CUUU | XX |
| WV-43147 | mU*fG*mUmAmAfUmAmGmCmCmAmGmUfAmCf | UGUAAUAGCCAGUACAGUU | XXOOOOOOOOOOOOOOOOOO |
| | AmGmUmUmCmC*mU*mU | CCUU | XX |
| WV-43148 | mU*fA*mGmUmGfGmGmAmGmUmCmGmGfAmUf | UAGUGGGAGUCGGAUUAU | XXOOOOOOOOOOOOOOOOOO |
| | UmAmUmUmUmU*mU*mU | UUUUU | XX |
| WV-43149 | mU*fG*mAmUmGfUmAmGmUmGmGmGmAfGmUf | UGAUGUAGUGGGAGUCGG | XXOOOOOOOOOOOOOOOOOO |
| | CmGmGmAmUmU*mU*mU | AUUUU | XX |
| WV-43150 | mU*fU*mGmAmUfGmUmAmGmUmGmGmGfAmGf | UUGAUGUAGUGGGAGUCG | XXOOOOOOOOOOOOOOOOOO |
| | UmCmGmGmAmU*mU*mU | GAUUU | XX |
| WV-43151 | mU*fU*mUmAmGfUmCmUmUmGmAmUmGfUmAf | UUUAGUCUUGAUGUAGUG | XXOOOOOOOOOOOOOOOOOO |
| | GmUmGmGmGmA*mU*mU | GGAUU | XX |
| WV-43152 | mU*fA*mUmUmAfGmUmCmUmUmGmAmUfGmUf | UAUUAGUCUUGAUGUAGU | XXOOOOOOOOOOOOOOOOOO |
| | AmGmUmGmGmG*mU*mU | GGGUU | XX |
| WV-43153 | mU*fG*mAmUmUfAmGmUmCmUmUmGmAfUmGf | UGAUUAGUCUUGAUGUAG | XXOOOOOOOOOOOOOOOOOO |
| | UmAmGmUmGmG*mU*mU | UGGUU | XX |
| WV-43154 | mU*fA*mGmAmUfUmAmGmUmCmUmUmGfAmUf | UAGAUUAGUCUUGAUGUA | XXOOOOOOOOOOOOOOOOOO |
| | GmUmAmGmUmG*mU*mU | GUGUU | XX |
| WV-43155 | mU*fC*mAmAmGfAmUmUmAmGmUmCmUfUmGf | UCAAGAUUAGUCUUGAUG | XXOOOOOOOOOOOOOOOOOO |
| | AmUmGmUmAmG*mU*mU | UAGUU | XX |
| WV-43156 | mU*fA*mCmAmUfGmUmGmAmAmAmAmAfCmAfC | UACAUGUGAAAAACACACA | XXOOOOOOOOOOOOOOOOOO |
| | mAmCmAmAmA*mU*mU | AAUU | XX |
| WV-43157 | mU*fA*mAmUmAfCmAmUmGmUmGmAmAfAmAf | UAAUACAUGUGAAAAACAC | XXOOOOOOOOOOOOOOOOOO |
| | AmCmAmCmAmC*mU*mU | ACUU | XX |
| WV-43158 | mU*fC*mUmAmUfAmAmUmAmCmAmUmGfUmGf | UCUAUAAUACAUGUGAAAA | XXOOOOOOOOOOOOOOOOOO |
| | AmAmAmAmAmC*mU*mU | ACUU | XX |
| WV-43159 | mU*fC*mCmAmUfGmCmAmAmAmAmGmCfAmUf | UCCAUGCAAAAGCAUUCUA | XXOOOOOOOOOOOOOOOOOO |
| | UmCmUmAmUmA*mU*mU | UAUU | XX |
| WV-43160 | mU*fU*mCmCmAfUmGmCmAmAmAmAmGfCmAf | UUCCAUGCAAAAGCAUUCU | XXOOOOOOOOOOOOOOOOOO |
| | UmUmCmUmAmU*mU*mU | AUUU | XX |
| WV-43161 | mU*fG*mUmCmCfAmUmGmCmAmAmAmAfGmCfA | UGUCCAUGCAAAAGCAUUC | XXOOOOOOOOOOOOOOOOOO |
| | mUmUmCmUmA*mU*mU | UAUU | XX |
| WV-43162 | mU*fA*mGmUmCfCmAmUmGmCmAmAmAfAmGfC | UAGUCCAUGCAAAAGCAUU | XXOOOOOOOOOOOOOOOOOO |
| | mAmUmUmCmU*mU*mU | CUUU | XX |
| WV-43163 | mU*fU*mAmGmUfCmCmAmUmGmCmAmAfAmAf | UUAGUCCAUGCAAAAGCAU | XXOOOOOOOOOOOOOOOOOO |
| | GmCmAmUmUmC*mU*mU | UCUU | XX |
| WV-43164 | mU*fG*mAmUmAfGmUmCmCmAmUmGmCfAmAf | UGAUAGUCCAUGCAAAAGC | XXOOOOOOOOOOOOOOOOOO |
| | AmAmGmCmAmU*mU*mU | AUUU | XX |
| WV-43165 | mU*fG*mGmAmUfAmGmUmCmCmAmUmGfCmAf | UGGAUAGUCCAUGCAAAAG | XXOOOOOOOOOOOOOOOOOO |
| | AmAmAmGmCmA*mU*mU | CAUU | XX |
| WV-43166 | mU*fA*mGmAmGfGmAmUmAmGmUmCmCfAmUf | UAGAGGAUAGUCCAUGCAA | XXOOOOOOOOOOOOOOOOOO |
| | GmCmAmAmAmA*mU*mU | AAUU | XX |
| WV-43167 | mU*fA*mAmGmAfGmGmAmUmAmGmUmCfCmAf | UAAGAGGAUAGUCCAUGCA | XXOOOOOOOOOOOOOOOOOO |
| | UmGmCmAmAmA*mU*mU | AAUU | XX |
| WV-43168 | mU*fC*mAmAmGfAmGmGmAmUmAmGmUfCmCf | UCAAGAGGAUAGUCCAUGC | XXOOOOOOOOOOOOOOOOOO |
| | AmUmGmCmAmA*mU*mU | AAUU | XX |
| WV-43169 | mU*fA*mAmCmAfAmGmAmGmGmAmUmAfGmUf | UAACAAGAGGAUAGUCCAU | XXOOOOOOOOOOOOOOOOOO |
| | CmCmAmUmGmC*mU*mU | GCUU | XX |
| WV-43170 | mU*fA*mAmAmCfAmAmGmAmGmGmAmUfAmGf | UAAACAAGAGGAUAGUCCA | XXOOOOOOOOOOOOOOOOOO |
| | UmCmCmAmUmG*mU*mU | UGUU | XX |
| WV-43171 | mU*fU*mAmAmUfAmAmAmAmAmCmAmAfGmAf | UUAAUAAAAACAAGAGGAU | XXOOOOOOOOOOOOOOOOOO |
| | GmGmAmUmAmG*mU*mU | AGUU | XX |
| WV-43172 | mU*fU*mUmAmAfUmAmAmAmAmAmCmAfAmGf | UUUAAUAAAAACAAGAGGA | XXOOOOOOOOOOOOOOOOOO |
| | AmGmGmAmUmA*mU*mU | UAUU | XX |
| WV-43173 | mU*fU*mAmAmCfUmAmCmAmAmGmAmGfGmUf | UUAACUACAAGAGGUUAU | XXOOOOOOOOOOOOOOOOOO |
| | UmAmUmUmUmU*mU*mU | UUUUU | XX |
| WV-43174 | mU*fU*mUmAmUfAmAmCmUmAmCmAmAfGmAf | UUUAUAACUACAAGAGGU | XXOOOOOOOOOOOOOOOOOO |
| | GmGmUmUmAmU*mU*mU | UAUUU | XX |
| WV-43175 | L025L025mA*mUmGmUmGmGfUmAfGfAfCmUmG | AUGUGGUAGACUGCAGCAA | OOXOOOOOOOOOOOOOOOOO |
| | mCmAmGmCmAmAmC*mA | CA | OX |
| WV-43176 | L025L025mG*mUmGmGmUmAfGmAfCfUfGmCmA | GUGGUAGACUGCAGCAACA | OOXOOOOOOOOOOOOOOOOO |
| | mGmCmAmAmCmAmG*mA | GA | OX |
| WV-43177 | L025L025mA*mAmCmAmUmCfCmUfAfGfGmAmCm | AACAUCCUAGGACAUUUUU | OOXOOOOOOOOOOOOOOOOO |
| | AmUmUmUmUmUmG*mA | GA | OX |
| WV-43178 | L025L025mU*mAmAmAmUmCfGmUfAfUfGmCmA | UAAAUCGUAUGCAGAAUAU | OOXOOOOOOOOOOOOOOOOO |
| | mGmAmAmUmAmUmU*mA | UA | OX |
| WV-43179 | L025L025mA*mUmCmGmUmAfUmGfCfAfGmAmA | AUCGUAUGCAGAAUAUUCA | OOXOOOOOOOOOOOOOOOOO |
| | mUmAmUmUmCmAmA*mA | AA | OX |
| WV-43180 | L025L025mC*mGmUmAmUmGfCmAfGfAfAmUmA | CGUAUGCAGAAUAUUCAAU | OOXOOOOOOOOOOOOOOOOO |
| | mUmUmCmAmAmUmU*mA | UA | OX |
| WV-43181 | L025L025mU*mAmUmGmCmAfGmAfAfUfAmUmU | UAUGCAGAAUAUUCAAUU | OOXOOOOOOOOOOOOOOOOO |
| | mCmAmAmUmUmUmG*mA | UGA | OX |
| WV-43182 | L025L025mU*mGmCmAmAmCfUmCfUfAfUmUmC | UGCAACUCUAUUCUGGACU | OOXOOOOOOOOOOOOOOOOO |
| | mUmGmGmAmCmUmU*mA | UA | OX |
| WV-43183 | L025L025mC*mAmAmCmUmCfUmAfUfUfCmUmG | CAACUCUAUUCUGGACUUU | OOXOOOOOOOOOOOOOOOOO |
| | mGmAmCmUmUmUmA*mA | AA | OX |
| WV-43184 | L025L025mA*mUmUmCmUmGfGmAfCfUfUmUmA | AUUCUGGACUUUAUUACU | OOXOOOOOOOOOOOOOOOOO |
| | mUmUmAmCmUmUmG*mA | UGA | OX |
| WV-43185 | L025L025mU*mCmCmAmCmCfAmAfAfAfGmUmGm | UCCACCAAAAGUGGACCCU | OOXOOOOOOOOOOOOOOOOO |
| | GmAmCmCmCmUmC*mA | CA | OX |
| WV-43186 | L025L025mC*mCmAmCmCmAfAmAfAfGfUmGmGm | CCACCAAAAGUGGACCCUC | OOXOOOOOOOOOOOOOOOOO |
| | AmCmCmCmUmCmU*mA | UA | OX |
| WV-43187 | L025L025mA*mCmCmAmAmAfAmGfUfGfGmAmCm | ACCAAAAGUGGACCCUCUA | OOXOOOOOOOOOOOOOOOOO |
| | CmCmUmCmUmAmU*mA | UA | OX |
| WV-43188 | L025L025mC*mCmAmAmAmAfGmUfGfGfAmCmCm | CCAAAAGUGGACCCUCUAU | OOXOOOOOOOOOOOOOOOOO |
| | CmUmCmUmAmUmA*mA | AA | OX |
| WV-43189 | L025L025mC*mCmCmUmCmUfAmUfAfUfUmUmC | CCCUCUAUAUUUCCUCCCU | OOXOOOOOOOOOOOOOOOOO |
| | mCmUmCmCmCmUmU*mA | UA | OX |
| WV-43190 | L025L025mA*mGmAmCmAmAfUmUfUfUfAmGmG | AGACAAUUUUAGGCUCAAA | OOXOOOOOOOOOOOOOOOOO |
| | mCmUmCmAmAmAmA*mA | AA | OX |
| WV-43191 | L025L025mA*mGmGmAmAmCfUmGfUfAfCmUmG | AGGAACUGUACUGGCUAU | OOXOOOOOOOOOOOOOOOOO |
| | mGmCmUmAmUmUmA*mA | UAA | OX |
| WV-43192 | L025L025mG*mGmAmAmCmUfGmUfAfCfUmGmG | GGAACUGUACUGGCUAUU | OOXOOOOOOOOOOOOOOOOO |
| | mCmUmAmUmUmAmC*mA | ACA | OX |
| WV-43193 | L025L025mA*mAmAmAmUmAfAmUfCfCfGmAmCm | AAAAUAAUCCGACUCCCAC | OOXOOOOOOOOOOOOOOOOO |
| | UmCmCmCmAmCmU*mA | UA | OX |
| WV-43194 | L025L025mA*mAmUmCmCmGfAmCfUfCfCmCmAm | AAUCCGACUCCCACUACAUC | OOXOOOOOOOOOOOOOOOOO |
| | CmUmAmCmAmUmC*mA | A | OX |
| WV-43195 | L025L025mA*mUmCmCmGmAfCmUfCfCfCmAmCm | AUCCGACUCCCACUACAUCA | OOXOOOOOOOOOOOOOOOOO |
| | UmAmCmAmUmCmA*mA | A | OX |
| WV-43196 | L025L025mU*mCmCmCmAmCfUmAfCfAfUmCmAm | UCCCACUACAUCAAGACUA | OOXOOOOOOOOOOOOOOOOO |
| | AmGmAmCmUmAmA*mA | AA | OX |
| WV-43197 | L025L025mC*mCmCmAmCmUfAmCfAfUfCmAmAm | CCCACUACAUCAAGACUAA | OOXOOOOOOOOOOOOOOOOO |
| | GmAmCmUmAmAmU*mA | UA | OX |
| WV-43198 | L025L025mC*mCmAmCmUmAfCmAfUfCfAmAmGm | CCACUACAUCAAGACUAAU | OOXOOOOOOOOOOOOOOOOO |
| | AmCmUmAmAmUmC*mA | CA | OX |
| WV-43199 | L025L025mC*mAmCmUmAmCfAmUfCfAfAmGmAm | CACUACAUCAAGACUAAUC | OOXOOOOOOOOOOOOOOOOO |
| | CmUmAmAmUmCmU*mA | UA | OX |
| WV-43200 | L025L025mC*mUmAmCmAmUfCmAfAfGfAmCmUm | CUACAUCAAGACUAAUCUU | OOXOOOOOOOOOOOOOOOOO |
| | AmAmUmCmUmUmG*mA | GA | OX |
| WV-43201 | L025L025mU*mUmUmGmUmGfUmGfUfUfUmUmU | UUUGUGUGUUUUUCACAU | OOXOOOOOOOOOOOOOOOOO |
| | mCmAmCmAmUmGmU*mA | GUA | OX |
| WV-43202 | L025L025mG*mUmGmUmGmUfUmUfUfUfCmAmC | GUGUGUUUUUCACAUGUA | OOXOOOOOOOOOOOOOOOOO |
| | mAmUmGmUmAmUmU*mA | UUA | OX |
| WV-43203 | L025L025mG*mUmUmUmUmUfCmAfCfAfUmGmU | GUUUUUCACAUGUAUUAU | OOXOOOOOOOOOOOOOOOOO |
| | mAmUmUmAmUmAmG*mA | AGA | OX |
| WV-43204 | L025L025mU*mAmUmAmGmAfAmUfGfCfUmUmU | UAUAGAAUGCUUUUGCAU | OOXOOOOOOOOOOOOOOOOO |
| | mUmGmCmAmUmGmG*mA | GGA | OX |
| WV-43205 | L025L025mA*mUmAmGmAmAfUmGfCfUfUmUmU | AUAGAAUGCUUUUGCAUG | OOXOOOOOOOOOOOOOOOOO |
| | mGmCmAmUmGmGmA*mA | GAA | OX |
| WV-43206 | L025L025mU*mAmGmAmAmUfGmCfUfUfUmUmG | UAGAAUGCUUUUGCAUGG | OOXOOOOOOOOOOOOOOOOO |
| | mCmAmUmGmGmAmC*mA | ACA | OX |
| WV-43207 | L025L025mA*mGmAmAmUmGfCmUfUfUfUmGmC | AGAAUGCUUUUGCAUGGA | OOXOOOOOOOOOOOOOOOOO |
| | mAmUmGmGmAmCmU*mA | CUA | OX |
| WV-43208 | L025L025mG*mAmAmUmGmCfUmUfUfUfGmCmA | GAAUGCUUUUGCAUGGAC | OOXOOOOOOOOOOOOOOOOO |
| | mUmGmGmAmCmUmA*mA | UAA | OX |
| WV-43209 | L025L025mA*mUmGmCmUmUfUmUfGfCfAmUmG | AUGCUUUUGCAUGGACUA | OOXOOOOOOOOOOOOOOOOO |
| | mGmAmCmUmAmUmC*mA | UCA | OX |
| WV-43210 | L025L025mU*mGmCmUmUmUfUmGfCfAfUmGmG | UGCUUUUGCAUGGACUAU | OOXOOOOOOOOOOOOOOOOO |
| | mAmCmUmAmUmCmC*mA | CCA | OX |
| WV-43211 | L025L025mU*mUmUmUmGmCfAmUfGfGfAmCmU | UUUUGCAUGGACUAUCCUC | OOXOOOOOOOOOOOOOOOOO |
| | mAmUmCmCmUmCmU*mA | UA | OX |
| WV-43212 | L025L025mU*mUmUmGmCmAfUmGfGfAfCmUmA | UUUGCAUGGACUAUCCUCU | OOXOOOOOOOOOOOOOOOOO |
| | mUmCmCmUmCmUmU*mA | UA | OX |
| WV-43213 | L025L025mU*mUmGmCmAmUfGmGfAfCfUmAmU | UUGCAUGGACUAUCCUCUU | OOXOOOOOOOOOOOOOOOOO |
| | mCmCmUmCmUmUmG*mA | GA | OX |
| WV-43214 | L025L025mG*mCmAmUmGmGfAmCfUfAfUmCmC | GCAUGGACUAUCCUCUUGU | OOXOOOOOOOOOOOOOOOOO |
| | mUmCmUmUmGmUmU*mA | UA | OX |
| WV-43215 | L025L025mC*mAmUmGmGmAfCmUfAfUfCmCmU | CAUGGACUAUCCUCUUGUU | OOXOOOOOOOOOOOOOOOOO |
| | mCmUmUmGmUmUmU*mA | UA | OX |
| WV-43216 | L025L025mC*mUmAmUmCmCfUmCfUfUfGmUmU | CUAUCCUCUUGUUUUUAU | OOXOOOOOOOOOOOOOOOOO |
| | mUmUmUmAmUmUmA*mA | UAA | OX |
| WV-43217 | L025L025mU*mAmUmCmCmUfCmUfUfGfUmUmU | UAUCCUCUUGUUUUUAUU | OOXOOOOOOOOOOOOOOOOO |
| | mUmUmAmUmUmAmA*mA | AAA | OX |
| WV-43218 | L025L025mA*mAmAmAmUmAfAmCfCfUfCmUmU | AAAAUAACCUCUUGUAGUU | OOXOOOOOOOOOOOOOOOOO |
| | mGmUmAmGmUmUmA*mA | AA | OX |
| WV-43219 | L025L025mA*mUmAmAmCmCfUmCfUfUfGmUmA | AUAACCUCUUGUAGUUAUA | OOXOOOOOOOOOOOOOOOOO |
| | mGmUmUmAmUmAmA*mA | AA | OX |
| WV-44171 | L001mC*mGmUmAmUmGfCmAfGfAfAmUmAmUm | CGUAUGCAGAAUAUUCAAU | OXOOOOOOOOOOOOOOOOOO |
| | UmCmAmAmUmU*mA | UA | X |
| WV-44172 | L001mU+mAmUmGmCmAfGmAfAfUfAmUmUmCm | UAUGCAGAAUAUUCAAUU | OXOOOOOOOOOOOOOOOOOO |
| | AmAmUmUmUmG*mA | UGA | X |
| WV-44173 | Mod001L001mC*mGmUmAmUmGfCmAfGfAfAmU | CGUAUGCAGAAUAUUCAAU | OXOOOOOOOOOOOOOOOOOO |
| | mAmUmUmCmAmAmUmU*mA | UA | X |
| WV-44174 | Mod001L001mUmAmUmGmCmAfGmAfAfUfAmU | UAUGCAGAAUAUUCAAUU | OXOOOOOOOOOOOOOOOOOO |
| | mUmCmAmAmUmUmUmG*mA | UGA | X |
| WV-45021 | mU*RfG*SmUmGmAfUmCmCmAmAmAmAmAfUm | UGUGAUCCAAAAAUGUCCU | RSOOOOOOOOOOOOOOOOOO |
| | GfUmCmCmUmAmG*SmU*SmU | AGUU | SS |
| WV-45022 | mU*SfG*RmUmGmAfUmCmCmAmAmAmAmAfUm | UGUGAUCCAAAAAUGUCCU | SROOOOOOOOOOOOOOOOOO |
| | GfUmCmCmUmAmG*SmU*SmU | AGUU | SS |
| WV-45023 | mU*RfG*SmUmGmAfUmCmCmAmAmAmAmAfUm | UGUGAUCCAAAAAUGUCCU | RSOOOOOOOOOOOOOOOOOO |
| | GfUmCmCmUmAmG*SmUn001RmU | AGUU | SnR |
| WV-45024 | mU*SfG*RmUmGmAfUmCmCmAmAmAmAmAfUm | UGUGAUCCAAAAAUGUCCU | RSOOOOOOOOOOOOOOOOOO |
| | GfUmCmCmUmAmG*SmUn001RmU | AGUU | SnR |
| WV-45025 | mU*RfG*SmUmGmAfUmCmCmAmAmAmAmAfUm | UGUGAUCCAAAAAUGUCCU | RSOOOOOOOOOOOOOOOOOO |
| | GfUmCmCmUmAmG*SmUn001SmU | AGUU | SnS |
| WV-45026 | mU*SfG*RmUmGmAfUmCmCmAmAmAmAmAfUm | UGUGAUCCAAAAAUGUCCU | SROOOOOOOOOOOOOOOOOO |
| | GfUmCmCmUmAmG*SmUn001SmU | AGUU | SnS |
| WV-45027 | mU*RfG*SmUmGmAfUmCmCmAmAmAmAmAfUm | UGUGAUCCAAAAAUGUCCU | RSOOOOOOOOOOOOOOOOOO |
| | GfUmCmCmUmAmGmUn001RmU | AGUU | OnR |
| WV-45028 | mU*SfG*RmUmGmAfUmCmCmAmAmAmAmAfUm | UGUGAUCCAAAAAUGUCCU | SROOOOOOOOOOOOOOOOOO |
| | GfUmCmCmUmAmGmUn001RmU | AGUU | OnR |
| WV-45029 | mU*RfG*SmUmGmAfUmCmCmAmAmAmAmAfUm | UGUGAUCCAAAAAUGUCCU | RSOOOOOOOOOOOOOOOOOO |
| | GfUmCmCmUmAmGmUn001SmU | AGUU | OnS |
| WV-45030 | mU*SfG*RmUmGmAfUmCmCmAmAmAmAmAfUm | UGUGAUCCAAAAAUGUCCU | SROOOOOOOOOOOOOOOOOO |
| | GfUmCmCmUmAmGmUn001SmU | AGUU | OnS |
| WV-45031 | mU*RfG*SmUmGmAfUmCfCmAmAmAfAmAfUmGf | UGUGAUCCAAAAAUGUCCU | RSOOOOOOOOOOOOOOOOOO |
| | UmCmCmUmAmG*SmU*SmU | AGUU | SS |
| WV-45032 | mU*SfG*RmUmGmAfUmCfCmAmAmAfAmAfUmGf | UGUGAUCCAAAAAUGUCCU | SROOOOOOOOOOOOOOOOOO |
| | UmCmCmUmAmG*SmU*SmU | AGUU | SS |
| WV-45033 | mU*RfG*SmUmGmAfUmCfCmAmAmAfAmAfUmGf | UGUGAUCCAAAAAUGUCCU | RSOOOOOOOOOOOOOOOOOO |
| | UmCmCmUmAmG*SmUn001RmU | AGUU | SnR |
| WV-45034 | mU*SfG*RmUmGmAfUmCfCmAmAmAfAmAfUmGf | UGUGAUCCAAAAAUGUCCU | SROOOOOOOOOOOOOOOOOO |
| | UmCmCmUmAmG*SmUn001RmU | AGUU | SnR |
| WV-45035 | mU*RfG*SmUmGmAfUmCfCmAmAmAfAmAfUmGf | UGUGAUCCAAAAAUGUCCU | RSOOOOOOOOOOOOOOOOOO |
| | UmCmCmUmAmG*SmUn001SmU | AGUU | SnS |
| WV-45036 | mU*SfG*RmUmGmAfUmCfCmAmAmAfAmAfUmGf | UGUGAUCCAAAAAUGUCCU | SROOOOOOOOOOOOOOOOOO |
| | UmCmCmUmAmG*SmUn001SmU | AGUU | SnS |
| WV-45037 | mU*RfG*SmUmGmAfUmCfCmAmAmAfAmAfUmGf | UGUGAUCCAAAAAUGUCCU | RSOOOOOOOOOOOOOOOOOO |
| | UmCmCmUmAmGmUn001RmU | AGUU | OnR |
| WV-45038 | mU*SfG*RmUmGmAfUmCfCmAmAmAfAmAfUmGf | UGUGAUCCAAAAAUGUCCU | SROOOOOOOOOOOOOOOOOO |
| | UmCmCmUmAmGmUn001RmU | AGUU | OnR |
| WV-45039 | mU*RfG*SmUmGmAfUmCfCmAmAmAfAmAfUmGf | UGUGAUCCAAAAAUGUCCU | RSOOOOOOOOOOOOOOOOOO |
| | UmCmCmUmAmGmUn001SmU | AGUU | OnS |
| WV-45040 | mU*SfG*RmUmGmAfUmCfCmAmAmAfAmAfUmGf | UGUGAUCCAAAAAUGUCCU | SROOOOOOOOOOOOOOOOOO |
| | UmCmCmUmAmGmUn001SmU | AGUU | OnS |
| WV-45041 | mU*RfG*SmAmCmAfUmGmAmGmGmUmUmUfUm | UGACAUGAGGUUUUGAUA | RSOOOOOOOOOOOOOOOOOO |
| | GfAmUmAmCmCmA*SmU*SmU | CCAUU | SS |
| WV-45042 | mU*SfG*RmAmCmAfUmGmAmGmGmUmUmUfUm | UGACAUGAGGUUUUGAUA | SROOOOOOOOOOOOOOOOOO |
| | GfAmUmAmCmCmA*SmU*SmU | CCAUU | SS |
| WV-45043 | mU*RfG*SmAmCmAfUmGmAmGmGmUmUmUfUm | UGACAUGAGGUUUUGAUA | SROOOOOOOOOOOOOOOOOO |
| | GfAmUmAmCmCmA*SmUn001RmU | CCAUU | SnR |
| WV-45044 | mU*SfG*RmAmCmAfUmGmAmGmGmUmUmUfUm | UGACAUGAGGUUUUGAUA | SROOOOOOOOOOOOOOOOOO |
| | GfAmUmAmCmCmA*SmUn001RmU | CCAUU | SnR |
| WV-45045 | mU*RfG*SmAmCmAfUmGmAmGmGmUmUmUfUm | UGACAUGAGGUUUUGAUA | RSOOOOOOOOOOOOOOOOOO |
| | GfAmUmAmCmCmA*SmUn001SmU | CCAUU | SnS |
| WV-45046 | mU*SfG*RmAmCmAfUmGmAmGmGmUmUmUfUm | UGACAUGAGGUUUUGAUA | SROOOOOOOOOOOOOOOOOO |
| | GfAmUmAmCmCmA*SmUn001SmU | CCAUU | SnS |
| WV-45047 | mU*RfG*SmAmCmAfUmGmAmGmGmUmUmUfUm | UGACAUGAGGUUUUGAUA | RSOOOOOOOOOOOOOOOOOO |
| | GfAmUmAmCmCmAmUn001RmU | CCAUU | OnR |
| WV-45048 | mU*SfG*RmAmCmAfUmGmAmGmGmUmUmUfUm | UGACAUGAGGUUUUGAUA | SROOOOOOOOOOOOOOOOOO |
| | GfAmUmAmCmCmAmUn001RmU | CCAUU | OnR |
| WV-45049 | mU*RfG*SmAmCmAfUmGmAmGmGmUmUmUfUm | UGACAUGAGGUUUUGAUA | RSOOOOOOOOOOOOOOOOOO |
| | GfAmUmAmCmCmAmUn001SmU | CCAUU | OnS |
| WV-45050 | mU*SfG*RmAmCmAfUmGmAmGmGmUmUmUfUm | UGACAUGAGGUUUUGAUA | SROOOOOOOOOOOOOOOOOO |
| | GfAmUmAmCmCmAmUn001SmU | CCAUU | OnS |
| WV-45051 | mU*RfG*SmAmCmAfUmGfAmGmGmUfUmUfUmGf | UGACAUGAGGUUUUGAUA | RSOOOOOOOOOOOOOOOOOO |
| | AmUmAmCmCmA*SmU*SmU | CCAUU | SS |
| WV-45052 | mU*SfG*RmAmCmAfUmGfAmGmGmUfUmUfUmGf | UGACAUGAGGUUUUGAUA | SROOOOOOOOOOOOOOOOOO |
| | AmUmAmCmCmA*SmU*SmU | CCAUU | SS |
| WV-45053 | mU*RfG*SmAmCmAfUmGfAmGmGmUfUmUfUmGf | UGACAUGAGGUUUUGAUA | RSOOOOOOOOOOOOOOOOOO |
| | AmUmAmCmCmA*SmUn001RmU | CCAUU | SnR |
| WV-45054 | mU*SfG*RmAmCmAfUmGfAmGmGmUfUmUfUmGf | UGACAUGAGGUUUUGAUA | SROOOOOOOOOOOOOOOOOO |
| | AmUmAmCmCmA*SmUn001RmU | CCAUU | SnR |
| WV-45055 | mU*RfG*SmAmCmAfUmGfAmGmGmUfUmUfUmGf | UGACAUGAGGUUUUGAUA | RSOOOOOOOOOOOOOOOOOO |
| | AmUmAmCmCmA*SmUn001SmU | CCAUU | SnS |
| WV-45056 | mU*SfG*RmAmCmAfUmGfAmGmGmUfUmUfUmGf | UGACAUGAGGUUUUGAUA | SROOOOOOOOOOOOOOOOOO |
| | AmUmAmCmCmA*SmUn001SmU | CCAUU | SnS |
| WV-45057 | mU*RfG*SmAmCmAfUmGfAmGmGmUfUmUfUmGf | UGACAUGAGGUUUUGAUA | RSOOOOOOOOOOOOOOOOOO |
| | AmUmAmCmCmAmUn001RmU | CCAUU | OnR |
| WV-45058 | mU*SfG*RmAmCmAfUmGfAmGmGmUfUmUfUmGf | UGACAUGAGGUUUUGAUA | SROOOOOOOOOOOOOOOOOO |
| | AmUmAmCmCmAmUn001RmU | CCAUU | OnR |
| WV-45059 | mU*RfG*SmAmCmAfUmGfAmGmGmUfUmUfUmGf | UGACAUGAGGUUUUGAUA | RSOOOOOOOOOOOOOOOOOO |
| | AmUmAmCmCmAmUn001SmU | CCAUU | OnS |
| WV-45060 | mU*SfG*RmAmCmAfUmGfAmGmGmUfUmUfUmGf | UGACAUGAGGUUUUGAUA | SROOOOOOOOOOOOOOOOOO |
| | AmUmAmCmCmAmUn001SmU | CCAUU | OnS |
| WV-46471 | mU*RfG*SmUn001SmGmAfUmCmCmAmAn001Sm | UGUGAUCCAAAAAUGUCCU | RSnSOOOOOOnSOOOOOOOOnS |
| | AmAmAfUmGfUmCmCmUn001SmAmG*SmU*SmU | AGUU | OSS |
| WV-46472 | mU*RfG*SmUmGmAfUmCmCmAmAn001SmAmAm | UGUGAUCCAAAAAUGUCCU | RSOOOOOOOnSOOOOOOOOnS |
| | AfUmGfUmCmCmUn001SmAmG*SmU*SmU | AGUU | OSS |
| WV-46473 | mU*RfG*SmUn001SmGmAfUmCmCmAmAmAmAm | UGUGAUCCAAAAAUGUCCU | RSnSOOOOOOOOOOOOOOOnS |
| | AfUmGfUmCmCmUn001SmAmG*SmU*SmU | AGUU | OSS |
| WV-46474 | mU*RfG*SmUn001SmGmAfUmCmCmAmAn001Sm | UGUGAUCCAAAAAUGUCCU | RSnSOOOOOOnSOOOOOOOOO |
| | AmAmAfUmGfUmCmCmUmAmG*SmU*SmU | AGUU | OSS |
| WV-46475 | mU*RfG*SmUmGmAfUmCmCmAmAmAmAmAfUm | UGUGAUCCAAAAAUGUCCU | RSOOOOOOOOOOOOOOOOnSO |
| | GfUmCmCmUn001SmAmG*SmU*SmU | AGUU | SS |
| WV-46476 | mU*RfG*SmUmGmAfUmCmCmAmAn001SmAmAm | UGUGAUCCAAAAAUGUCCU | RSOOOOOOOnSOOOOOOOOOO |
| | AfUmGfUmCmCmUmAmG*SmU*SmU | AGUU | SS |
| WV-46477 | mU*RfG*SmUn001SmGmAfUmCmCmAmAmAmAm | UGUGAUCCAAAAAUGUCCU | RSnSOOOOOOOOOOOOOOOOO |
| | AfUmGfUmCmCmUmAmG*SmU*SmU | AGUU | SS |
| WV-46478 | mU*SfG*RmUmGmAfUmCmCmAmAn001SmAmAm | UGUGAUCCAAAAAUGUCCU | SROOOOOOOnSOOOOOOOOnS |
| | AfUmGfUmCmCmUn001SmAmG*SmU*SmU | AGUU | OSS |
| WV-46479 | mU*SfG*RmUn001SmGmAfUmCmCmAmAmAmAm | UGUGAUCCAAAAAUGUCCU | SRnSOOOOOOOOOOOOOOOnS |
| | AfUmGfUmCmCmUn001SmAmG*SmU*SmU | AGUU | OSS |
| WV-46480 | mU*SfG*RmUn001SmGmAfUmCmCmAmAn001Sm | UGUGAUCCAAAAAUGUCCU | SRnSOOOOOOnSOOOOOOOOO |
| | AmAmAfUmGfUmCmCmUmAmG*SmU*SmU | AGUU | OSS |
| WV-46481 | mU*SfG*RmUmGmAfUmCmCmAmAmAmAmAfUm | UGUGAUCCAAAAAUGUCCU | SROOOOOOOOOOOOOOOOnSO |
| | GfUmCmCmUn001SmAmG*SmU*SmU | AGUU | SS |
| WV-46482 | mU*SfG*RmUmGmAfUmCmCmAmAn001SmAmAm | UGUGAUCCAAAAAUGUCCU | SROOOOOOOnSOOOOOOOOOO |
| | AfUmGfUmCmCmUmAmG*SmU*SmU | AGUU | SS |
| WV-46483 | mU*SfG*RmUn001SmGmAfUmCmCmAmAmAmAm | UGUGAUCCAAAAAUGUCCU | SRnSOOOOOOOOOOOOOOOOO |
| | AfUmGfUmCmCmUmAmG*SmU*SmU | AGUU | SS |
| WV-46484 | mU*RfG*SmAn001SmCmAfUmGmAmGmGn001Sm | UGACAUGAGGUUUUGAUA | RSnSOOOOOOnSOOOOOOOOnS |
| | UmUmUfUmGfAmUmAmCn001SmCmA*SmU*SmU | CCAUU | OSS |
| WV-46485 | mU*RfG*SmAmCmAfUmGmAmGmGn001SmUmU | UGACAUGAGGUUUUGAUA | RSOOOOOOOnSOOOOOOOOnS |
| | mUfUmGfAmUmAmCn001SmCmA*SmU*SmU | CCAUU | OSS |
| WV-46486 | mU*RfG*SmAn001SmCmAfUmGmAmGmGmUmU | UGACAUGAGGUUUUGAUA | RSnSOOOOOOOOOOOOOOOnS |
| | mUfUmGfAmUmAmCn001SmCmA*SmU*SmU | CCAUU | OSS |
| WV-46487 | mU*RfG*SmAn001SmCmAfUmGmAmGmGn001Sm | UGACAUGAGGUUUUGAUA | RSnSOOOOOOnSOOOOOOOOO |
| | UmUmUfUmGfAmUmAmCmCmA*SmU*SmU | CCAUU | OSS |
| WV-46488 | mU*RfG*SmAmCmAfUmGmAmGmGmUmUmUfUm | UGACAUGAGGUUUUGAUA | RSOOOOOOOOOOOOOOOOnSO |
| | GfAmUmAmCn001SmCmA*SmU*SmU | CCAUU | SS |
| WV-46489 | mU*RfG*SmAmCmAfUmGmAmGmGn001SmUmU | UGACAUGAGGUUUUGAUA | RSOOOOOOOnSOOOOOOOOOO |
| | mUfUmGfAmUmAmCmCmA*SmU*SmU | CCAUU | SS |
| WV-46490 | mU*RfG*SmAn001SmCmAfUmGmAmGmGmUmU | UGACAUGAGGUUUUGAUA | RSnSOOOOOOOOOOOOOOOOO |
| | mUfUmGfAmUmAmCmCmA*SmU*SmU | CCAUU | SS |
| WV-46491 | mU*SfG*RmAmCmAfUmGmAmGmGn001SmUmU | UGACAUGAGGUUUUGAUA | SROOOOOOOnSOOOOOOOOnS |
| | mUfUmGfAmUmAmCn001SmCmA*SmU*SmU | CCAUU | OSS |
| WV-46492 | mU*SfG*RmAn001SmCmAfUmGmAmGmGmUmU | UGACAUGAGGUUUUGAUA | SRnSOOOOOOOOOOOOOOOnS |
| | mUfUmGfAmUmAmCn001SmCmA*SmU*SmU | CCAUU | OSS |
| WV-46493 | mU*SfG*RmAn001SmCmAfUmGmAmGmGn001Sm | UGACAUGAGGUUUUGAUA | SRnSOOOOOOnSOOOOOOOOO |
| | UmUmUfUmGfAmUmAmCmCmA*SmU*SmU | CCAUU | OSS |
| WV-46494 | mU*SfG*RmAmCmAfUmGmAmGmGmUmUmUfUm | UGACAUGAGGUUUUGAUA | SROOOOOOOOOOOOOOOOnSO |
| | GfAmUmAmCn001SmCmA*SmU*SmU | CCAUU | SS |
| WV-46495 | mU*SfG*RmAmCmAfUmGmAmGmGn001SmUmU | UGACAUGAGGUUUUGAUA | SROOOOOOOnSOOOOOOOOOO |
| | mUfUmGfAmUmAmCmCmA*SmU*SmU | CCAUU | SS |
| WV-46496 | mU*SfG*RmAn001SmCmAfUmGmAmGmGmUmU | UGACAUGAGGUUUUGAUA | SRnSOOOOOOOOOOOOOOOOO |
| | mUfUmGfAmUmAmCmCmA*SmU*SmU | CCAUU | SS |
| WV-47138 | mU*RfG*SmAn001SfCmAfUmGmAmGmGmUmUm | UGACAUGAGGUUUUGAUA | RSnSOOOOOOOOOOOOOOOOO |
| | UfUmGfAmUmAmCmCmA*SmU*SmU | CCAUU | SS |
| WV-47139 | mU*SfG*RmAn001SfCmAfUmGmAmGmGmUmUm | UGACAUGAGGUUUUGAUA | SRnSOOOOOOOOOOOOOOOOO |
| | UfUmGfAmUmAmCmCmA*SmU*SmU | CCAUU | SS |
| WV-47140 | mU*SfG*RmUn001SfGmAfUmCmCmAmAmAmAmA | UGUGAUCCAAAAAUGUCCU | SRnSOOOOOOOOOOOOOOOOO |
| | fUmGfUmCmCmUmAmG*SmU*SmU | AGUU | SS |
| WV-47158 | mU*RfG*SmAn001SfCmAfUmGmAmGmGn001SfU | UGACAUGAGGUUUUGAUA | RSnSOOOOOOnSOOOOOOOOO |
| | mUmUfUmGfAmUmAmCmCmA*SmU*SmU | CCAUU | OSS |
| WV-47159 | mU*SfG*RmAn001SfCmAfUmGmAmGmGn001SfU | UGACAUGAGGUUUUGAUA | SRnSOOOOOOnSOOOOOOOOO |
| | mUmUfUmGfAmUmAmCmCmA*SmU*SmU | CCAUU | OSS |
| WV-49590 | mU*SfG*RmAn001SfCmAfUmGmAmGmGn001RfU | UGACAUGAGGUUUUGAUA | SRnSOOOOOOnROOOOOOOOO |
| | mUmUfUmGfAmUmAmCmCmA*SmU*SmU | CCAUU | OSS |
| WV-49591 | mU*SfG*RmAmCmAfUmGmAmGmGn001RfUmUm | UGACAUGAGGUUUUGAUA | SROOOOOOOnROOOOOOOOOO |
| | UfUmGfAmUmAmCmCmA*SmU*SmU | CCAUU | SS |
| WV-49592 | mU*fG*mAn001fCmAfUmGmAmGmGn001fUmUm | UGACAUGAGGUUUUGAUA | XXnXOOOOOOnXOOOOOOOOO |
| | UfUmGfAmUmAmCmCmA*mU*mU | CCAUU | OXX |
| WV-49593 | mU*fG*mAfCmAfUmGmAmGmGfUmUmUfUmGfA | UGACAUGAGGUUUUGAUA | XXOOOOOOOOOOOOOOOOOO |
| | mUmAmCmCmA*mU*mU | CCAUU | XX |
| WV-49614 | mU*SfC*RmCn001SfUmUfCmCmCmUmGn001RfAm | UCCUUCCCUGAAGGUUCCUCC | SRnSOOOOOOnROOOOOOOOO |
| | AmGfGmUfUmCmCmUmCmC*SmU*SmU | | OSS |
| WV-49615 | Mod0011001mG*SmGmAmGmGmAfAmCfCfUfUmCm | GGAGGAACCUUCAGGGAAGGA | OSOOOOOOOOOOOOOOOOOOS |
| | AmGmGmGmAmAmGmG*SmA | | |

### Notes:

Description, Base Sequence and Stereochemistry/Linkage, due to their length, may be divided into multiple lines in Table 1. Unless otherwise specified, all oligonucleotides in Table 1 are single-stranded. As appreciated by those skilled in the art, nucleoside units are unmodified and contain unmodified nucleobases and 2′-deoxy sugars unless otherwise indicated (e.g., with r, m, etc.); linkages, unless otherwise indicated, are natural phosphate linkages; and acidic/basic groups may independently exist in their salt forms. If a sugar is not specified, the sugar is a natural DNA sugar; and if an internucleotidic linkage is not specified, the internucleotidic linkage is a natural phosphate linkage. Moieties and modifications:
m: 2′-OMe;
f: 2′-F;
O, PO: phosphodiester (phosphate). It can a linkage or be an end group (or a component thereof), e.g., a linkage between a linker and an oligonucleotide chain, an internucleotidic linkage (a natural phosphate linkage), etc. Phosphodiesters are typically indicated with “0” in the Stereochemistry/Linkage column and are typically not marked in the Description column (if it is an end group, e.g., a 5′-end group, it is indicated in the Description and typically not in Stereochemistry/Linkage); if no linkage is indicated in the Description column, it is typically a phosphodiester unless otherwise indicated. Note that a phosphate linkage between a linker (e.g., L001) and an oligonucleotide chain may not be marked in the Description column, but may be indicated with “0” in the Stereochemistry/Linkage column; *, PS: Phosphorothioate. It can be an end group (if it is an end group, e.g., a 5′-end group, it is indicated in the Description and typically not in Stereochemistry/Linkage), or a linkage, e.g., a linkage between linker (e.g., L001) and an oligonucleotide chain, an internucleotidic linkage (a phosphorothioate internucleotidic linkage), etc.;
R, Rp: Phosphorothioate in the Rp configuration. Note that * R in Description indicates a single phosphorothioate linkage in the Rp configuration;
S, Sp: Phosphorothioate in the Sp configuration. Note that * S in Description indicates a single phosphorothioate linkage in the Sp configuration;
X: stereorandom phosphorothioate;

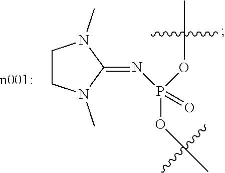 [SMILES: *OP(=O)(N=C1N(C)CCN1C)O*]

nX: stereorandom n001;
nR or n001R: n001 in Rp configuration;
nS or n001S: n001 in Sp configuration;

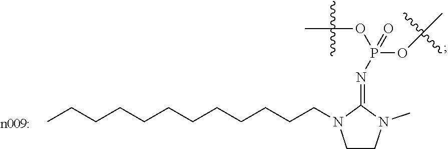 [SMILES: *OP(=O)(/N=C1\N(C)CCN1CCCCCCCCCCCC)O*]

nX: stereorandom n009;
nR or n009R: n009 in Rp configuration;
nS or n009S: n009 in S configuration;

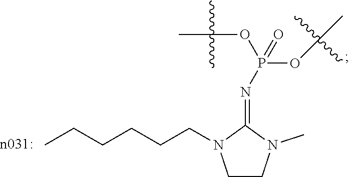 [SMILES: *OP(=O)(/N=C1\N(C)CCN1CCCCCC)O*]

nX: stereorandom n031;
nR or n031R: n031 in Rp configuration;
nS or n031S: n031 in Sp configuration;

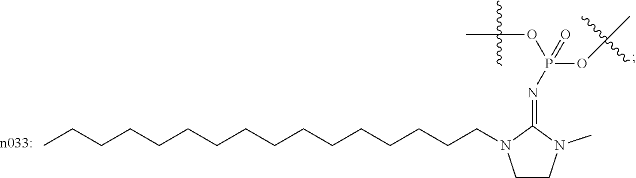 [SMILES: *OP(=O)(/N=C1\N(C)CCN1CCCCCCCCCCCCCCCC)O*]

nX: stereorandom n033;
nR or n033R: n033 in Rp configuration;
nS or n033S: n033 in Sp configuration

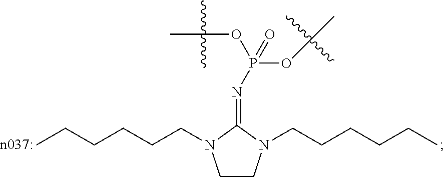 [SMILES: *OP(=O)(N=C1N(CCCCCC)CCN1CCCCCC)O*]

nX: stereorandom n037;
nR or n037R: n037 in Rp configuration;
nS or n037S: n037 in Sp configuration;

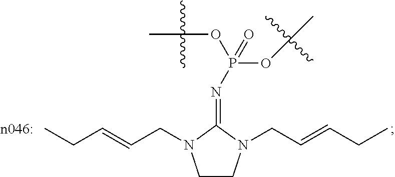 [SMILES: *OP(=O)(N=C1N(C/C=C/CC)CCN1C/C=C/CC)O*]

nX: stereorandom n046;
nR or n046R: n046 in Rp configuration;
nS or n046S: n046 in Sp configuration;

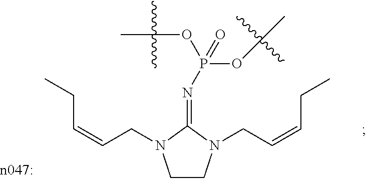 [SMILES: *OP(=O)(N=C1N(C/C=C\CC)CCN1C/C=C\CC)O*]

nX: stereorandom n047;
nR or n047R: n047 in Rp configuration;
nS or n047S: n047 in Sp configuration;

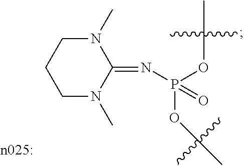 [SMILES: *OP(=O)(N=C1N(C)CCCN1C)O*]

nX: stereorandom n025;
nR or n025R: n025 in Rp configuration;
nS or n025S: n025 in Sp configuration;

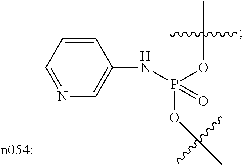 [SMILES: *OP(=O)(Nc1cccnc1)O*]

nX: stereorandom n054;
nR or n054R: n054 in Rp configuration;
nS or n054S: n054 in Sp configuration;

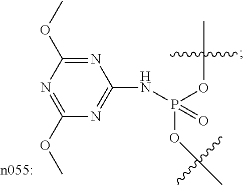 [SMILES: *OP(=O)(Nc1nc(OC)nc(OC)n1)O*]

nX: stereorandom n055;
nR or n055R: n055 in Rp configuration;
nS or n055S: n055 in Sp configuration;

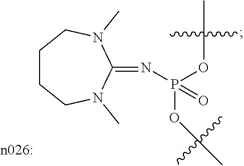 [SMILES: *OP(=O)(N=C1N(C)CCCCN1C)O*]

nX: stereorandom n001;
nR or n026R: n026 in Rp configuration;
nS or n026S: n026 in Sp configuration;

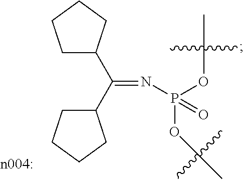 [SMILES: *OP(=O)(N=C(C1CCCC1)C1CCCC1)O*]

nX: stereorandom n004;
nR or n004R: n004 in Rp configuration;
nS or n004S: n004 in Sp configuration;

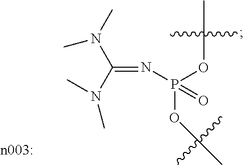 [SMILES: *OP(=O)(N=C(N(C)C)N(C)C)O*]

nX: stereorandom n003;
nR or n003R: n003 in Rp configuration;
nS or n003S: n003 in Sp configuration;

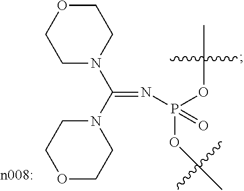 [SMILES: *OP(=O)(N=C(N1CCOCC1)N1CCOCC1)O*]

nX: stereorandom n008;
nR or n008R: n008 in Rp configuration;
nS or n008S: n008 in Sp configuration;

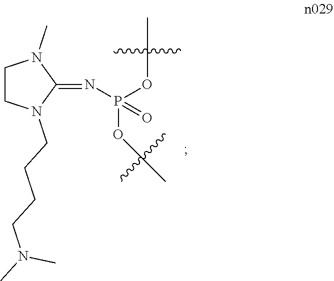 [SMILES: *OP(=O)(/N=C1/N(C)CCN1CCCCN(C)C)O*]

nX: stereorandom n029;
nR or n029R: n029 in Rp configuration;
nS or n029S: n029 in Sp configuration;

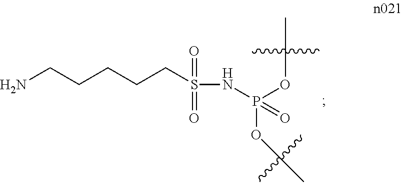 [SMILES: *OP(=O)(NS(=O)(=O)CCCCCN)O*]

nX: stereorandom n021;
nR or n021R: n021 in Rp configuration;
nS or n021S: n021 in Sp configuration;

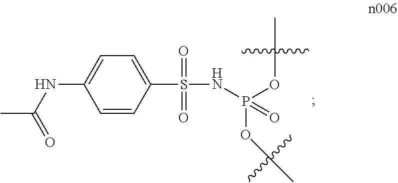 [SMILES: *OP(=O)(NS(=O)(=O)c1ccc(NC(C)=O)cc1)O*]

nX: stereorandom n006;
nR or n006R: n006 in Rp configuration;
nS or n006S: n006 in Sp configuration;

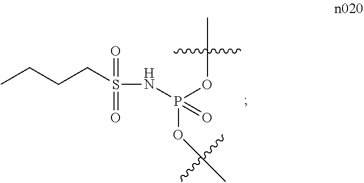 [SMILES: *OP(=O)(NS(=O)(=O)CCCC)O*]

nX: stereorandom n020;
nR or n020R: n020 in Rp configuration;
nS or n020S: n020 in Sp configuration;
X: stereorandom phosphorothioate;

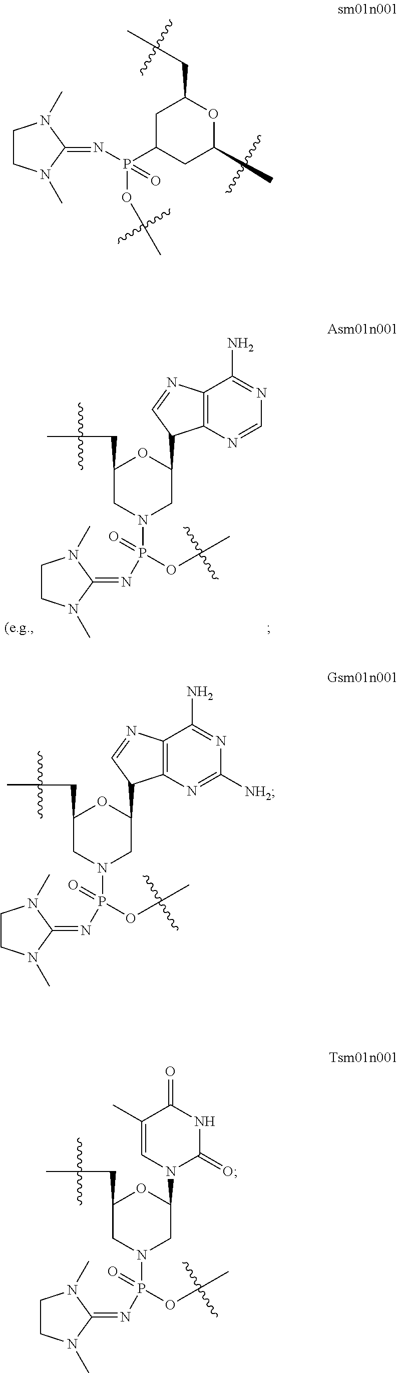 [SMILES: *C[C@@H]1CC(P(=O)(N=C2N(C)CCN2C)O*)C[C@H](C)O1 ; *C[C@@H]1CN(P(=O)(N=C2N(C)CCN2C)O*)C[C@H](C2C=Nc3c(N)ncnc32)O1 ; *C[C@@H]1CN(P(=O)(N=C2N(C)CCN2C)O*)C[C@H](C2C=Nc3c(N)nc(N)nc32)O1 ; *C[C@@H]1CN(P(=O)(N=C2N(C)CCN2C)O*)C[C@H](n2cc(C)c(=O)[nH]c2=O)O1]

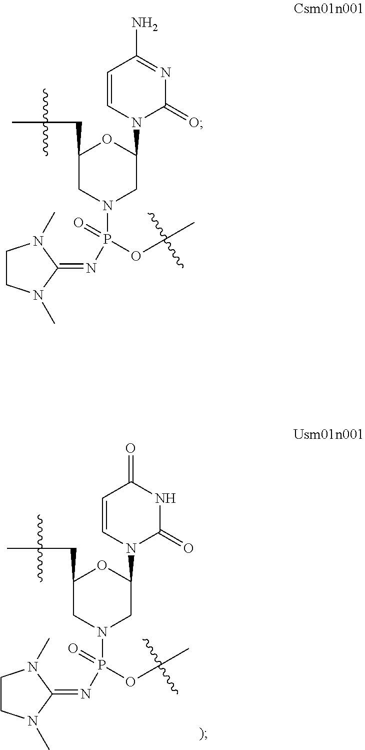 [SMILES: *C[C@@H]1CN(P(=O)(N=C2N(C)CCN2C)O*)C[C@H](n2ccc(N)nc2=O)O1 ; *C[C@@H]1CN(P(=O)(N=C2N(C)CCN2C)O*)C[C@H](n2ccc(=O)[nH]c2=O)O1]

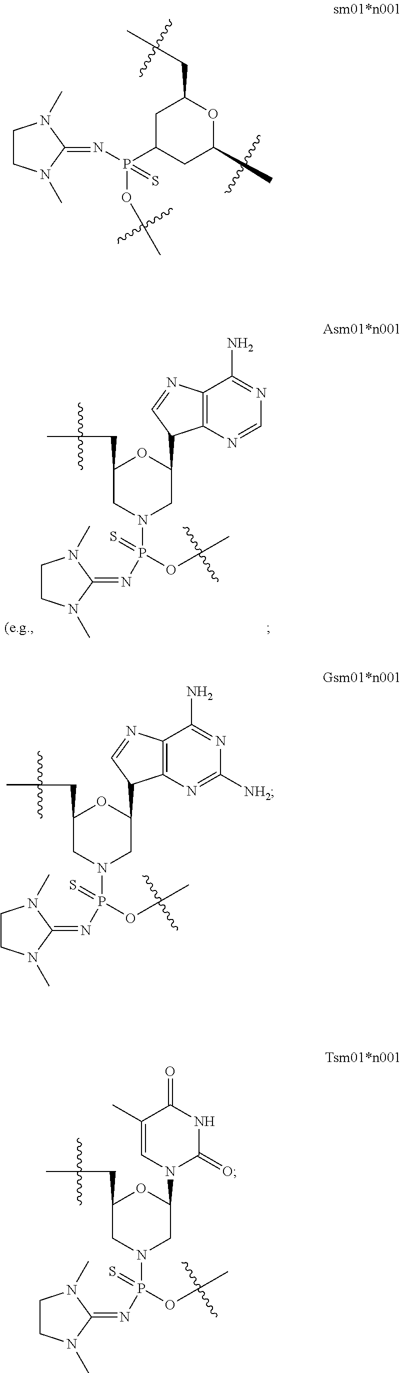 [SMILES: *C[C@@H]1CC(P(=S)(N=C2N(C)CCN2C)O*)C[C@H](C)O1 ; *C[C@@H]1CN(P(=S)(N=C2N(C)CCN2C)O*)C[C@H](C2C=Nc3c(N)ncnc32)O1 ; *C[C@@H]1CN(P(=S)(N=C2N(C)CCN2C)O*)C[C@H](C2C=Nc3c(N)nc(N)nc32)O1 ; *C[C@@H]1CN(P(=S)(N=C2N(C)CCN2C)O*)C[C@H](n2cc(C)c(=O)[nH]c2=O)O1]

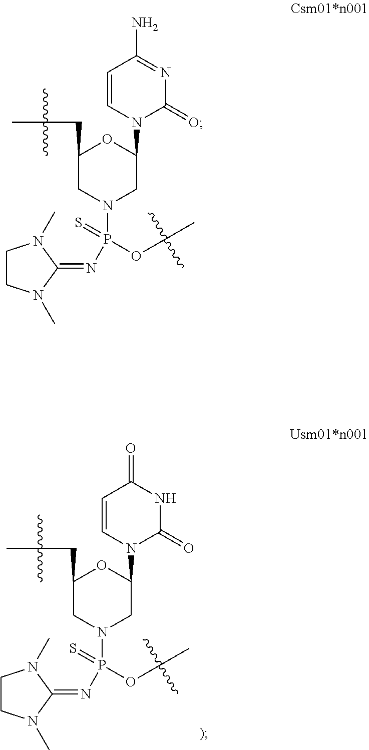 [SMILES: *C[C@@H]1CN(P(=S)(N=C2N(C)CCN2C)O*)C[C@H](n2ccc(N)nc2=O)O1 ; *C[C@@H]1CN(P(=S)(N=C2N(C)CCN2C)O*)C[C@H](n2ccc(=O)[nH]c2=O)O1]

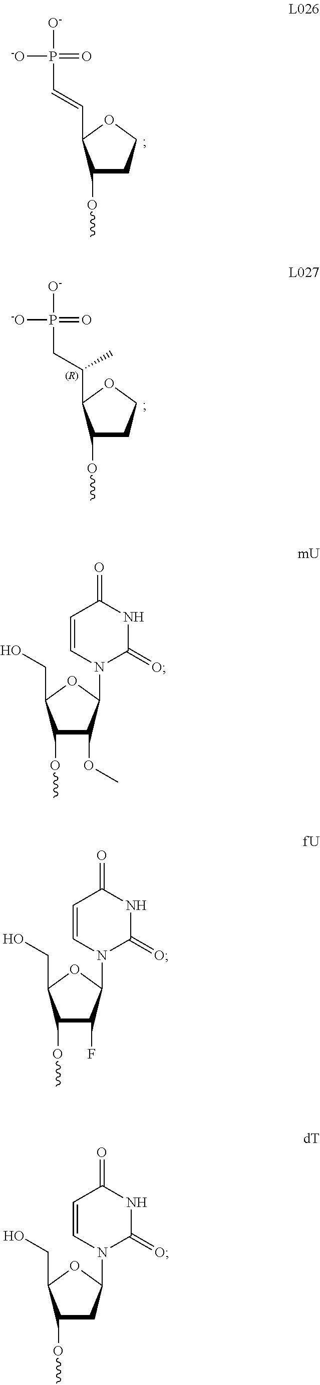

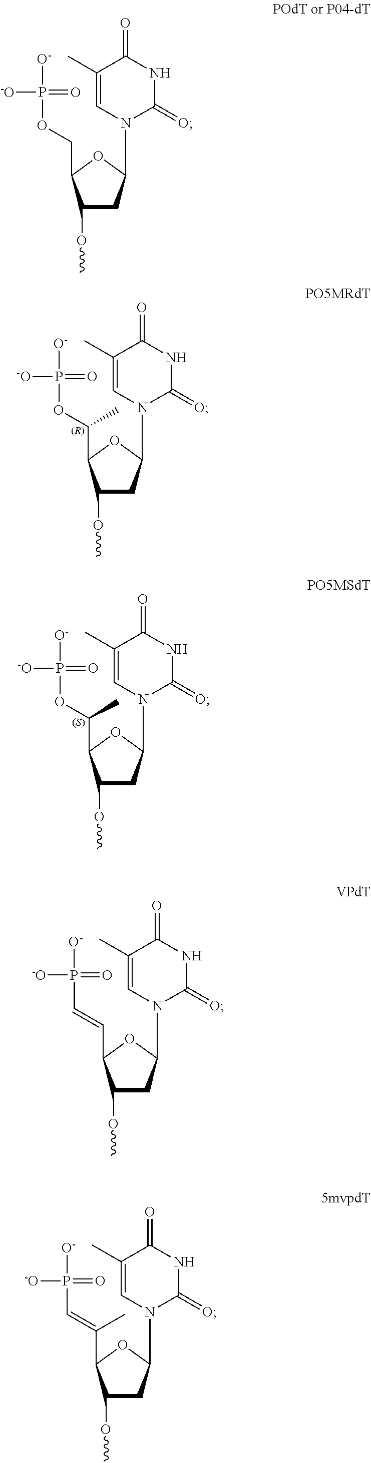

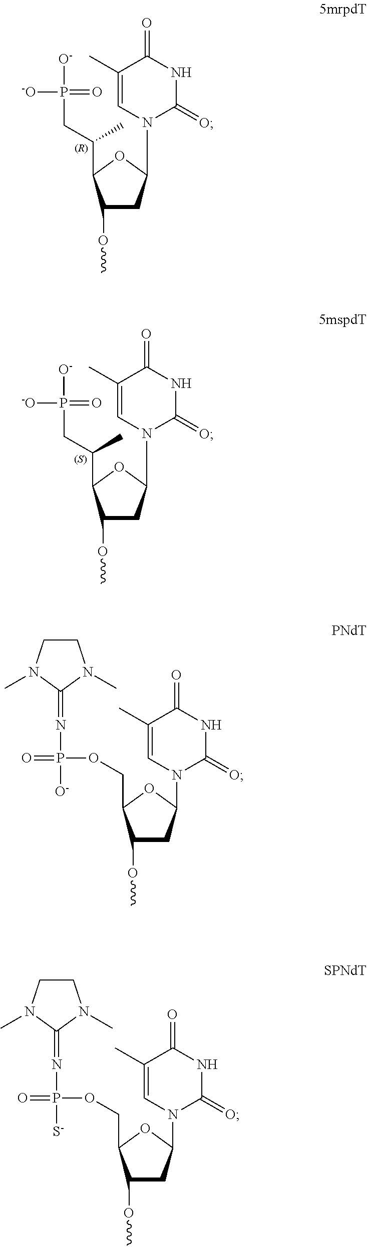

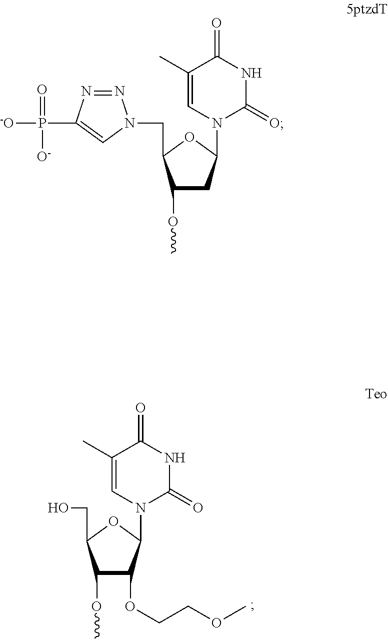

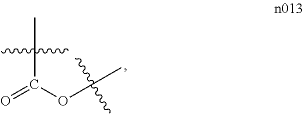 [SMILES: *OC(*)=O]

wherein —C(O)— is bonded to nitrogen;

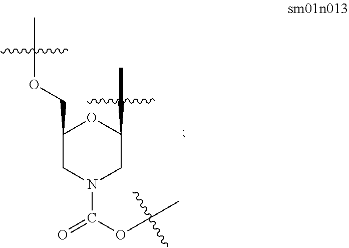 [SMILES: *OC[C@@H]1CN(C(=O)O*)C[C@H](*)O1]

i.e. morpholine carbamate internucleotidic linkages (sm01n013)

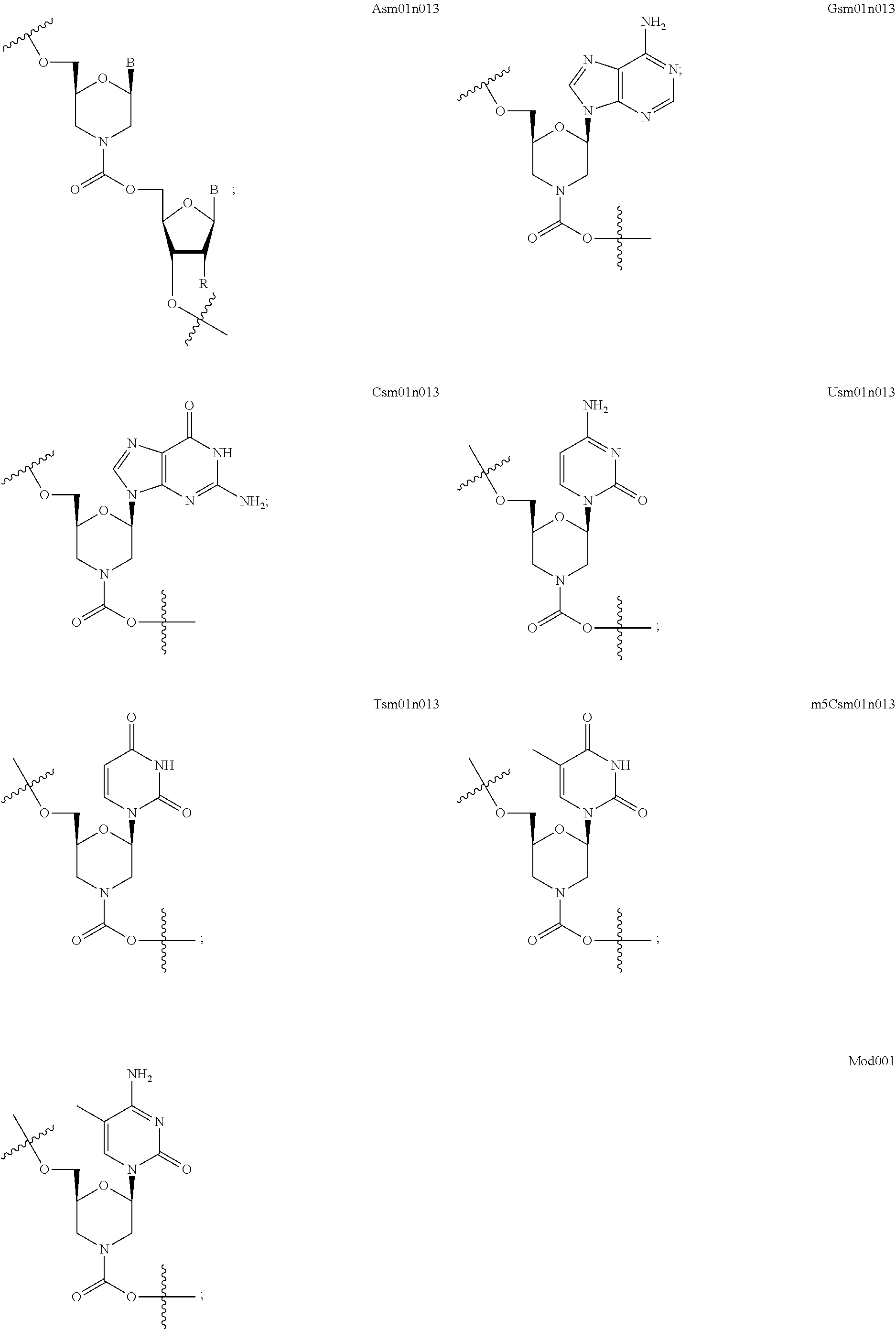

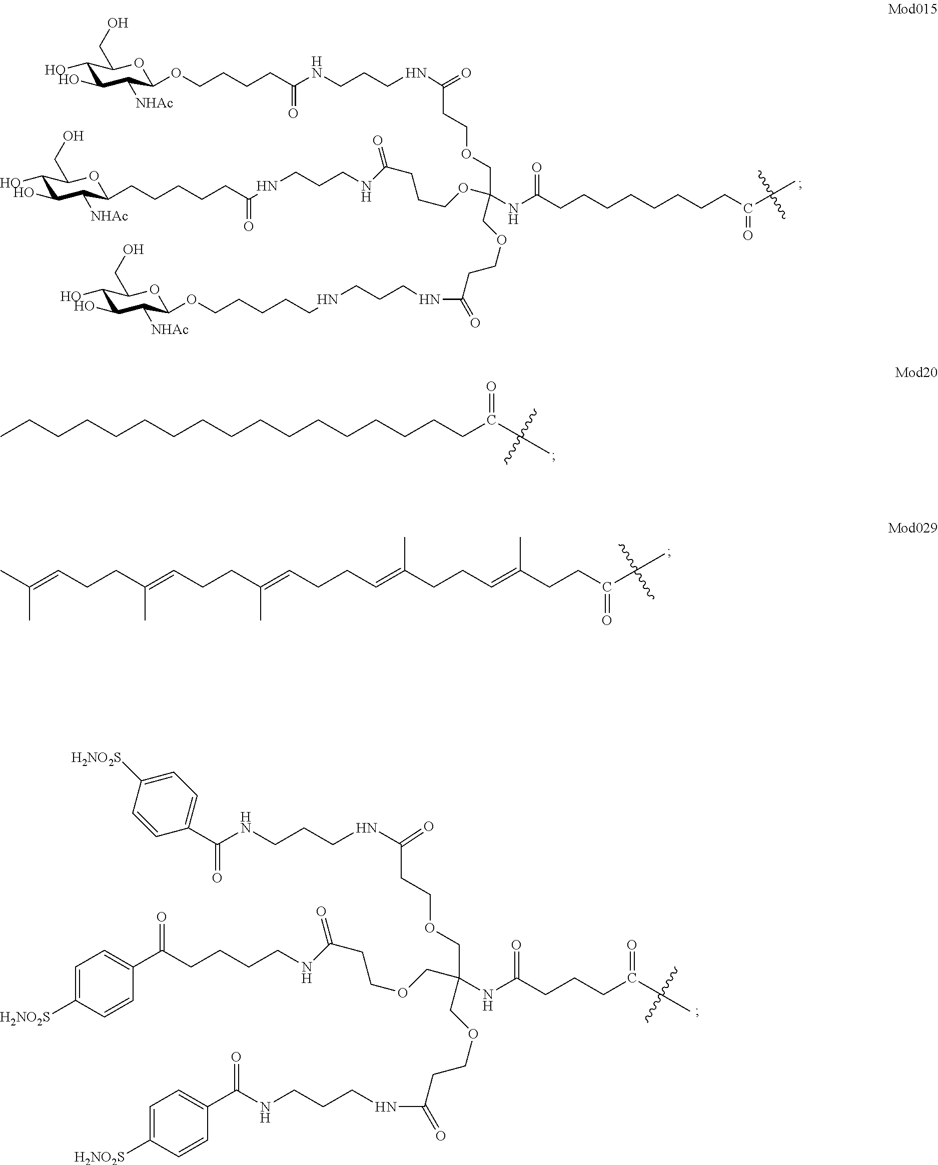

L001: —NH—(CH₂)₆— linker (C6 linker, C6 amine linker or C6 amino linker), connected to Mod (e.g., Mod001) through —NH—, and, in the case of, for example, WV-38061, the 5′-end of the oligonucleotide chain through a phosphate linkage (O or PO). For example, in WV-38061, L001 is connected to Mod001 through —NH— (forming an amide group —C(O)—NH—), and is connected to the oligonucleotide chain through a phosphate linkage (O).

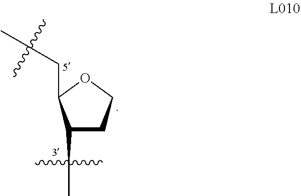

In some embodiments, when L010 is present in the middle of an oligonucleotide, it is bonded to internucleotidic linkages as other sugars (e.g., DNA sugars), e.g., its 5′-carbon is connected to another unit (e.g., 3′ of a sugar) and its 3′-carbon is connected to another unit (e.g., a 5′-carbon of a carbon) independently, e.g., via a linkage (e.g., a phosphate linkage (O or PO) or a phosphorothioate linkage (can be either not chirally controlled or chirally controlled (Sp or Rp)));
L012:—CH₂CH₂OCH₂CH₂OCH₂CH₂—. When L012 is present in the middle of an oligonucleotide, each of its two ends is independently bonded to an internucleotidic linkage (e.g., a phosphate linkage (O or PO) or a phosphorothioate linkage (can be either not chirally controlled or chirally controlled (Sp or Rp)));

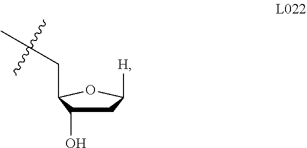

wherein L022 is connected to the rest of a molecule through a phosphate unless indicated otherwise;
L023: HO—(CH₂)₆—, wherein CH₂ is connected to the rest of a molecule through a phosphate unless indicated otherwise. For example, in WV-42644 (wherein the O in OnRnRnRnRSSSSSSSSSSSSSSSSSSnRSSSSSnRSSnR indicates a phosphate linkage connecting L023 to the rest of the molecule);

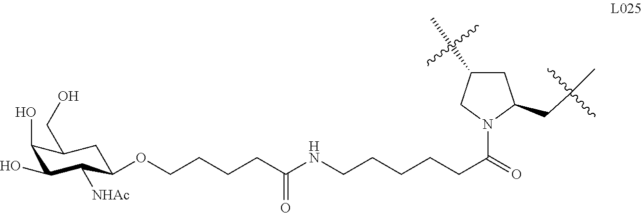

wherein the —CH₂— connection site is utilized as a C5 connection site of a sugar (e.g., a DNA sugar) and is connected to another unit (e.g., 3′ of a sugar), and the connection site on the ring is utilized as a C3 connection site and is connected to another unit (e.g., a 5′-carbon of a carbon), each of which is independently, e.g., via a linkage (e.g., a phosphate linkage (O or PO) or a phosphorothioate linkage (can be either not chirally controlled or chirally controlled (Sp or Rp))). When L025 is at a 5′-end without any modifications, its —CH₂— connection site is bonded to —OH. For example, L025L025L025—in various oligonucleotides has the structure of

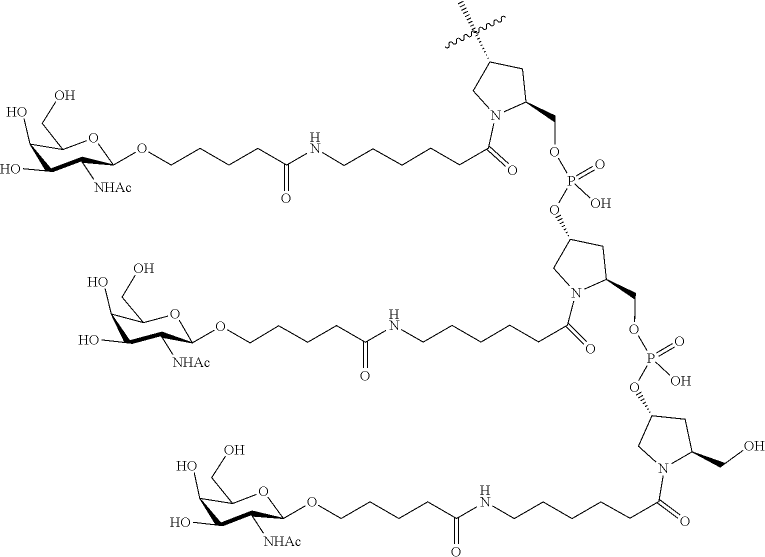

(may exist as various salt forms) and is connected to 5′-carbon of an oligonucleotide chain via a linkage as indicated (e.g., a phosphate linkage (O or PO) or a phosphorothioate linkage (can be either not chirally controlled or chirally controlled (Sp or Rp)));
L016:

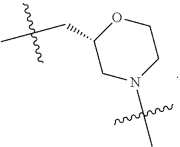

wherein L016 is connected to the rest of a molecule through a phosphate unless indicated otherwise; L016 is utilized with n001 to form L016n001, which has the structure of

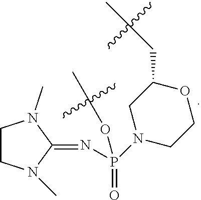 [SMILES: *C[C@H]1CN(P(=O)(N=C2N(C)CCN2C)O*)CCO1]

### Double Stranded Oligonucleotide Lengths

As appreciated by those skilled in the art, ds oligonucleotides targeting HSD17B13 can be of various lengths to provide desired properties and/or activities for various uses. Many technologies for assessing, selecting and/or optimizing oligonucleotide length are available in the art and can be utilized in accordance with the present disclosure. As demonstrated herein, in many embodiments, ds oligonucleotides targeting HSD17B13 are of suitable lengths to hybridize with their targets and reduce levels of their targets and/or an encoded product thereof. In some embodiments, an oligonucleotide is long enough to recognize a target nucleic acid (e.g., a HSD17B13 mRNA). In some embodiments, an oligonucleotide is sufficiently long to distinguish between a target nucleic acid and other nucleic acids (e.g., a nucleic acid having a base sequence which is not HSD17B13) to reduce off-target effects. In some embodiments, a ds oligonucleotide targeting HSD17B13 is sufficiently short to reduce complexity of manufacture or production and to reduce cost of products.

In some embodiments, the base sequence of an oligonucleotide is about 10-500 nucleobases in length. In some embodiments, a base sequence is about 10-500 nucleobases in length. In some embodiments, a base sequence is about 10-50 nucleobases in length. In some embodiments, a base sequence is about 15-50 nucleobases in length. In some embodiments, a base sequence is from about 15 to about 30 nucleobases in length. In some embodiments, a base sequence is from about 10 to about 25 nucleobases in length. In some embodiments, a base sequence is from about 15 to about 22 nucleobases in length. In some embodiments, a base sequence is about 10, 11, 12, 13, 14, 15, 16, 17, 18, 19, 20, 21, 22, 23, 24, or 25 nucleobases in length. In some embodiments, a base sequence is about 18 nucleobases in length. In some embodiments, a base sequence is about 19 nucleobases in length. In some embodiments, a base sequence is about 20 nucleobases in length. In some embodiments, a base sequence is about 21 nucleobases in length. In some embodiments, a base sequence is about 22 nucleobases in length. In some embodiments, a base sequence is about 23 nucleobases in length. In some embodiments, a base sequence is about 24 nucleobases in length. In some embodiments, a base sequence is about 25 nucleobases in length. In some embodiments, each nucleobase is optionally substituted A, T, C, G, U, or an optionally substituted tautomer of A, T, C, G, or U.

### Double Stranded Oligonucleotide Internucleotidic Linkages

In some embodiments, ds oligonucleotides targeting HSD17B13 comprise base modifications, sugar modifications, and/or internucleotidic linkage modifications. Various internucleotidic linkages can be utilized in accordance with the present disclosure to link units comprising nucleobases, e.g., nucleosides. In some embodiments, ds oligonucleotides targeting HSD17B13 comprise both one or more modified internucleotidic linkages and one or more natural phosphate linkages. As widely known by those skilled in the art, natural phosphate linkages are widely found in natural DNA and RNA molecules; they have the structure of —OP(O)(OH)O—, connect sugars in the nucleosides in DNA and RNA, and may be in various salt forms, for example, at physiological pH (about 7.4), natural phosphate linkages are predominantly exist in salt forms with the anion being —OP(O)(O⁻)O—. A modified internucleotidic linkage, or a non-natural phosphate linkage, is an internucleotidic linkage that is not natural phosphate linkage or a salt form thereof. Modified internucleotidic linkages, depending on their structures, may also be in their salt forms. For example, as appreciated by those skilled in the art, phosphorothioate internucleotidic linkages which have the structure of —OP(O)(SH)O— may be in various salt forms, e.g., at physiological pH (about 7.4) with the anion being —OP(O)(S⁻)O—.

In some embodiments, an oligonucleotide comprises an internucleotidic linkage which is a modified internucleotidic linkage, e.g., phosphorothioate, phosphorodithioate, methylphosphonate, phosphoroamidate, thiophosphate, 3′-thiophosphate, or 5′-thiophosphate. In some embodiments, a modified internucleotidic linkage is a chiral internucleotidic linkage which comprises a chiral linkage phosphorus. In some embodiments, a chiral internucleotidic linkage is a phosphorothioate linkage. In some embodiments, a chiral internucleotidic linkage is a non-negatively charged internucleotidic linkage. In some embodiments, a chiral internucleotidic linkage is a neutral internucleotidic linkage. In some embodiments, a chiral internucleotidic linkage is chirally controlled with respect to its chiral linkage phosphorus. In some embodiments, a chiral internucleotidic linkage is stereochemically pure with respect to its chiral linkage phosphorus. In some embodiments, a chiral internucleotidic linkage is not chirally controlled. In some embodiments, a pattern of backbone chiral centers comprises or consists of positions and linkage phosphorus configurations of chirally controlled internucleotidic linkages (Rp or Sp) and positions of achiral internucleotidic linkages (e.g., natural phosphate linkages).

In certain embodiments, an internucleotidic linkage comprises a P-modification, wherein a P-modification is a modification at a linkage phosphorus. In certain embodiments, a modified internucleotidic linkage is a moiety which does not comprise a phosphorus but serves to link two sugars or two moieties that each independently comprises a nucleobase, e.g., as in peptide nucleic acid (PNA).

In certain embodiments, a ds oligonucleotide comprises a modified internucleotidic linkage, e.g., those having the structure of Formula I, I-a, I-b, or I-c and described herein and/or in: WO 2018/022473, WO 2018/098264, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612, the internucleotidic linkages (e.g., those of Formula I, I-a, I-b, I-c, etc.) of each of which are independently incorporated herein by reference. In certain embodiments, a modified internucleotidic linkage is a chiral internucleotidic linkage. In certain embodiments, a modified internucleotidic linkage is a phosphorothioate internucleotidic linkage.

In certain embodiments, a modified internucleotidic linkage is anon-negatively charged internucleotidic linkage. In certain embodiments, provided ds oligonucleotides comprise one or more non-negatively charged internucleotidic linkages. In certain embodiments, a non-negatively charged internucleotidic linkage is a positively charged internucleotidic linkage. In certain embodiments, a non-negatively charged internucleotidic linkage is a neutral internucleotidic linkage. In certain embodiments, the present disclosure provides ds oligonucleotides comprising one or more neutral internucleotidic linkages. In certain embodiments, a non-negatively charged internucleotidic linkage has the structure of Formula I-n-1, I-n-2, I-n-3, I-n-4, II, II-a-1, II-a-2, II-b-1, II-b-2, II-c-1, II-c-2, II-d-1, II-d-2, etc., or a salt form thereof, as described herein and/or in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019/032612, WO 2019/055951, WO 2019/075357, WO 2019/200185, WO 2019/217784, and/or WO 2019/032612, the non-negatively charged internucleotidic linkages (e.g., those of Formula I-n-1, I-n-2, I-n-3, I-n-4, II, II-a-1, II-a-2, II-b-1, II-b-2, II-c-1, II-c-2, II-d-1, II-d-2, etc., or a suitable salt form thereof) of each of which are independently incorporated herein by reference.

In certain embodiments, a non-negatively charged internucleotidic linkage can improve the delivery and/or activities (e.g., adenosine editing activity).

In certain embodiments, a modified internucleotidic linkage (e.g., a non-negatively charged internucleotidic linkage) comprises optionally substituted triazolyl. In certain embodiments, a modified internucleotidic linkage (e.g., a non-negatively charged internucleotidic linkage) comprises optionally substituted alkynyl. In certain embodiments, a modified internucleotidic linkage comprises a triazole or alkyne moiety. In certain embodiments, a triazole moiety, e.g., a triazolyl group, is optionally substituted. In certain embodiments, a triazole moiety, e.g., a triazolyl group) is substituted. In certain embodiments, a triazole moiety is unsubstituted. In certain embodiments, a modified internucleotidic linkage comprises an optionally substituted cyclic guanidine moiety. In certain embodiments, a modified internucleotidic linkage has the structure of

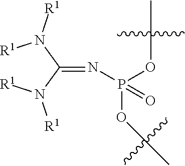

and is optionally chirally controlled, wherein R¹ is -L-R′, wherein L is L^{B} as described herein, and R′ is as described herein. In certain embodiments, each R¹ is independently R′. In certain embodiments, each R′ is independently R. In certain embodiments, two R¹ are R and are taken together to form a ring as described herein. In certain embodiments, two R¹ on two different nitrogen atoms are R and are taken together to form a ring as described herein. In certain embodiments, R¹ is independently optionally substituted C₁₋₆ aliphatic as described herein. In certain embodiments, R¹ is methyl. In certain embodiments, two R′ on the same nitrogen atom are R and are taken together to form a ring as described herein. In certain embodiments, a modified internucleotidic linkage has the structure of

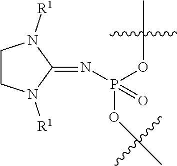

and is optionally chirally controlled. In certain embodiments,

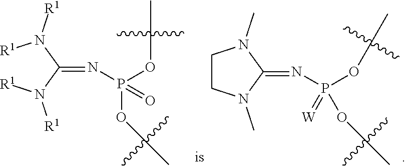

In certain embodiments, a modified internucleotidic linkage comprises an optionally substituted cyclic guanidine moiety and has the structure of:

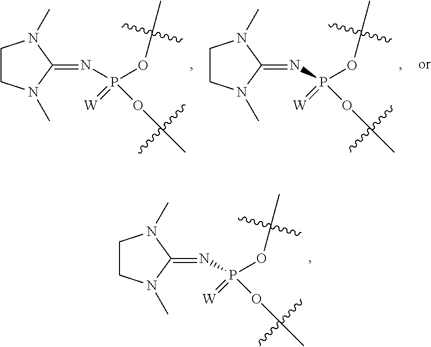

wherein W is O or S. In certain embodiments, W is O. In certain embodiments, W is S. In certain embodiments, a non-negatively charged internucleotidic linkage is stereochemically controlled.

In certain embodiments, a non-negatively charged internucleotidic linkage or a neutral internucleotidic linkage is an internucleotidic linkage comprising a triazole moiety. In some embodiments, an internucleotidic linkage comprising a triazole moiety (e.g., an optionally substituted triazolyl group) has the structure of

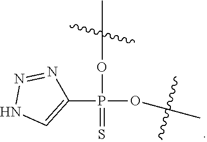 [SMILES: *OP(=S)(O*)c1c[nH]nn1]

In some embodiments, an internucleotidic linkage comprising a triazole moiety has the structure of

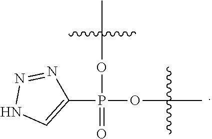 [SMILES: *OP(=O)(O*)c1c[nH]nn1]

In some embodiments, an internucleotidic linkage comprising a triazole moiety has the formula of

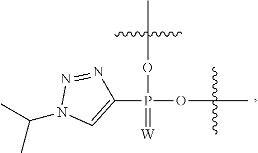

where W is O or S. In some embodiments, an internucleotidic linkage comprising an alkyne moiety (e.g., an optionally substituted alkynyl group) has the formula of

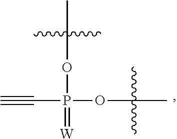

wherein W is O or S. In some embodiments, an internucleotidic linkage, e.g., a non-negatively charged internucleotidic linkage, a neutral internucleotidic linkage, comprises a cyclic guanidine moiety. In some embodiments, an internucleotidic linkage comprising a cyclic guanidine moiety has the structure of

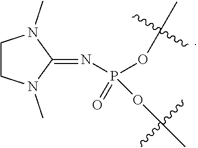 [SMILES: *OP(=O)(N=C1N(C)CCN1C)O*]

In some embodiments, a non-negatively charged internucleotidic linkage, or a neutral internucleotidic linkage, is or comprising a structure selected from

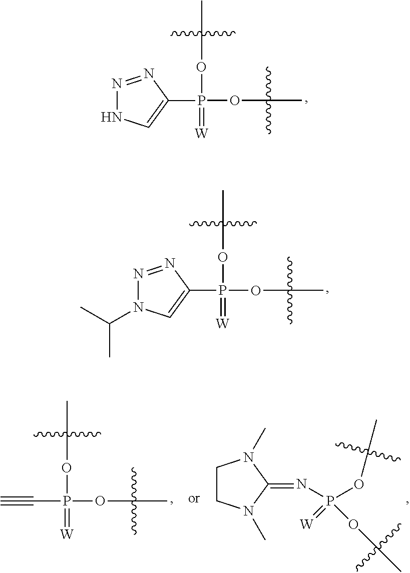

wherein W is O or S. In certain embodiments, an internucleotidic linkage, e.g., a non-negatively charged internucleotidic linkage, a neutral internucleotidic linkage, comprises a cyclic guanidine moiety. In certain embodiments, an internucleotidic linkage comprising a cyclic guanidine moiety has the structure of

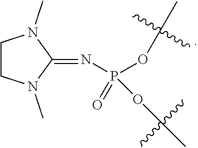 [SMILES: *OP(=O)(N=C1N(C)CCN1C)O*]

In certain embodiments, a non-negatively charged internucleotidic linkage, or a neutral internucleotidic linkage, is or comprising a structure

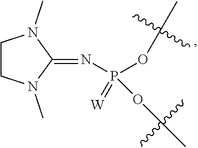

wherein W is O or S.

In certain embodiments, an internucleotidic linkage comprises a Tmg group (

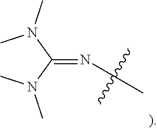 [SMILES: *N=C(N(C)C)N(C)C]

In certain embodiments, an internucleotidic linkage comprises a Tmg group and has the structure of

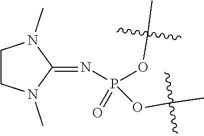 [SMILES: *OP(=O)(N=C1N(C)CCN1C)O*]

(the “Tmg internucleotidic linkage”). In certain embodiments, neutral internucleotidic linkages include internucleotidic linkages of PNA and PMO, and a Tmg internucleotidic linkage.

In certain embodiments, a non-negatively charged internucleotidic linkage has the structure of Formula I, I-a, I-b, I-c, I-n-1, I-n-2, I-n-3, I-n-4, II, II-a-1, II-a-2, II-b-1, II-b-2, II-c-1, II-c-2, II-d-1, II-d-2, etc., or a salt form thereof. In certain embodiments, a non-negatively charged internucleotidic linkage comprises an optionally substituted 3-20 membered heterocyclyl or heteroaryl group having 1-10 heteroatoms. In certain embodiments, a non-negatively charged internucleotidic linkage comprises an optionally substituted 3-20 membered heterocyclyl or heteroaryl group having 1-10 heteroatoms, wherein at least one heteroatom is nitrogen. In certain embodiments, such a heterocyclyl or heteroaryl group is of a 5-membered ring. In certain embodiments, such a heterocyclyl or heteroaryl group is of a 6-membered ring.

In certain embodiments, a non-negatively charged internucleotidic linkage comprises an optionally substituted 5-20 membered heteroaryl group having 1-10 heteroatoms. In certain embodiments, a non-negatively charged internucleotidic linkage comprises an optionally substituted 5-20 membered heteroaryl group having 1-10 heteroatoms, wherein at least one heteroatom is nitrogen. In certain embodiments, a non-negatively charged internucleotidic linkage comprises an optionally substituted 5-6 membered heteroaryl group having 1-4 heteroatoms, wherein at least one heteroatom is nitrogen. In certain embodiments, a non-negatively charged internucleotidic linkage comprises an optionally substituted 5-membered heteroaryl group having 1-4 heteroatoms, wherein at least one heteroatom is nitrogen. In certain embodiments, a heteroaryl group is directly bonded to a linkage phosphorus.

In certain embodiments, a non-negatively charged internucleotidic linkage comprises an optionally substituted 5-20 membered heterocyclyl group having 1-10 heteroatoms. In certain embodiments, a non-negatively charged internucleotidic linkage comprises an optionally substituted 5-20 membered heterocyclyl group having 1-10 heteroatoms, wherein at least one heteroatom is nitrogen. In certain embodiments, a non-negatively charged internucleotidic linkage comprises an optionally substituted 5-6 membered heterocyclyl group having 1-4 heteroatoms, wherein at least one heteroatom is nitrogen. In certain embodiments, a non-negatively charged internucleotidic linkage comprises an optionally substituted 5-membered heterocyclyl group having 1-4 heteroatoms, wherein at least one heteroatom is nitrogen. In certain embodiments, at least two heteroatoms are nitrogen. In some embodiments, a non-negatively charged internucleotidic linkage comprises an optionally substituted triazolyl group. In some embodiments, a non-negatively charged internucleotidic linkage comprises an unsubstituted triazolyl group, e.g.,

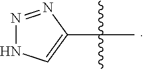 [SMILES: *c1c[nH]nn1]

In some embodiments, a non-negatively charged internucleotidic linkage comprises a substituted triazolyl group, e.g.,

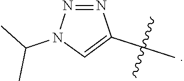 [SMILES: *c1cn(C(C)C)nn1]

In certain embodiments, a heterocyclyl group is directly bonded to a linkage phosphorus. In certain embodiments, a heterocyclyl group is bonded to a linkage phosphorus through a linker, e.g., ═N— when the heterocyclyl group is part of a guanidine moiety who directed bonded to a linkage phosphorus through its ═N—. In certain embodiments, a non-negatively charged internucleotidic linkage comprises an optionally substituted

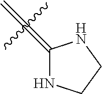 [SMILES: *=C1NCCN1]

group. In certain embodiments, a non-negatively charged internucleotidic linkage comprises an substituted

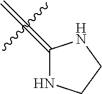 [SMILES: *=C1NCCN1]

group. In certain embodiments, a non-negatively charged internucleotidic linkage comprises a

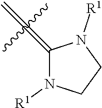

group, wherein each R¹ is independently -L-R. In certain embodiments, each R¹ is independently optionally substituted C₁₋₆ alkyl. In certain embodiments, each R¹ is independently methyl.

In certain embodiments, a modified internucleotidic linkage, e.g., a non-negatively charged internucleotidic linkage, comprises a triazole or alkyne moiety, each of which is optionally substituted. In certain embodiments, a modified internucleotidic linkage comprises a triazole moiety. In certain embodiments, a modified internucleotidic linkage comprises a unsubstituted triazole moiety. In certain embodiments, a modified internucleotidic linkage comprises a substituted triazole moiety. In certain embodiments, a modified internucleotidic linkage comprises an alkyl moiety. In certain embodiments, a modified internucleotidic linkage comprises an optionally substituted alkynyl group. In certain embodiments, a modified internucleotidic linkage comprises an unsubstituted alkynyl group. In certain embodiments, a modified internucleotidic linkage comprises a substituted alkynyl group. In certain embodiments, an alkynyl group is directly bonded to a linkage phosphorus.

In certain embodiments, a ds oligonucleotide comprises different types of internucleotidic phosphorus linkages. In certain embodiments, a chirally controlled oligonucleotide comprises at least one natural phosphate linkage and at least one modified (non-natural) internucleotidic linkage. In certain embodiments, a ds oligonucleotide comprises at least one natural phosphate linkage and at least one phosphorothioate. In certain embodiments, a ds oligonucleotide comprises at least one non-negatively charged internucleotidic linkage. In certain embodiments, a ds oligonucleotide comprises at least one natural phosphate linkage and at least one non-negatively charged internucleotidic linkage. In certain embodiments, a ds oligonucleotide comprises at least one phosphorothioate internucleotidic linkage and at least one non-negatively charged internucleotidic linkage. In certain embodiments, a ds oligonucleotide comprises at least one phosphorothioate internucleotidic linkage, at least one natural phosphate linkage, and at least one non-negatively charged internucleotidic linkage. In certain embodiments, ds oligonucleotides comprise one or more, e.g., 1-50, 1-40, 1-30, 1-20, 1-15, 1-10, 1, 2, 3, 4, 5, 6, 7, 8, 9, 10, 11, 12, 13, 14, 15, 16, 17, 18, 19, 20 or more non-negatively charged internucleotidic linkages. In certain embodiments, a non-negatively charged internucleotidic linkage is not negatively charged in that at a given pH in an aqueous solution less than 50%, 40%, 40%, 30%, 20%, 10%, 5%, or 1% of the internucleotidic linkage exists in a negatively charged salt form. In certain embodiments, a pH is about pH 7.4. In certain embodiments, a pH is about 4-9. In certain embodiments, the percentage is less than 10%. In certain embodiments, the percentage is less than 5%. In certain embodiments, the percentage is less than 1%. In certain embodiments, an internucleotidic linkage is a non-negatively charged internucleotidic linkage in that the neutral form of the internucleotidic linkage has no pKa that is no more than about 1, 2, 3, 4, 5, 6, or 7 in water. In certain embodiments, no pKa is 7 or less. In certain embodiments, no pKa is 6 or less. In certain embodiments, no pKa is 5 or less. In certain embodiments, no pKa is 4 or less. In certain embodiments, no pKa is 3 or less. In certain embodiments, no pKa is 2 or less. In certain embodiments, no pKa is 1 or less. In certain embodiments, pKa of the neutral form of an internucleotidic linkage can be represented by pKa of the neutral form of a compound having the structure of CH₃— the internucleotidic linkage —CH₃. For example, pKa of the neutral form of an internucleotidic linkage having the structure of Formula I may be represented by the pKa of the neutral form of a compound having the structure of

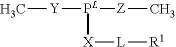

(wherein each of X, Y, Z is independently —O—, —S—, —N(R′)—; L is L^{B}, and R¹ is -L-R′), pKa of

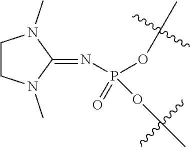 [SMILES: *OP(=O)(N=C1N(C)CCN1C)O*]

can be represented by pKa

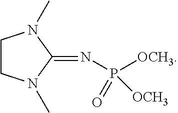

In certain embodiments, a non-negatively charged internucleotidic linkage is a neutral internucleotidic linkage. In certain embodiments, a non-negatively charged internucleotidic linkage is a positively-charged internucleotidic linkage. In certain embodiments, a non-negatively charged internucleotidic linkage comprises a guanidine moiety. In certain embodiments, a non-negatively charged internucleotidic linkage comprises a heteroaryl base moiety. In certain embodiments, a non-negatively charged internucleotidic linkage comprises a triazole moiety. In certain embodiments, a non-negatively charged internucleotidic linkage comprises an alkynyl moiety.

In certain embodiments, a neutral or non-negatively charged internucleotidic linkage has the structure of any neutral or non-negatively charged internucleotidic linkage described in any of: U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019/032612, WO 2019/055951, WO 2019/075357, WO 2019/200185, WO 2019/217784, and/or WO 2019/032612,2607, WO2019032612, WO 2019/055951, WO 2019/075357, WO 2019/200185, WO 2019/217784, and/or WO 2019/032612, each neutral or non-negatively charged internucleotidic linkage of each of which is hereby incorporated by reference.

In certain embodiments, each R′ is independently optionally substituted C₁₋₆ aliphatic. In certain embodiments, each R′ is independently optionally substituted C₁₋₆ alkyl. In certain embodiments, each R′ is independently —CH₃. In certain embodiments, each R^{s} is —H.

In certain embodiments, a non-negatively charged internucleotidic linkage has the structure of

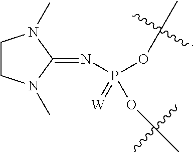

In certain embodiments, a non-negatively charged internucleotidic linkage has the structure of

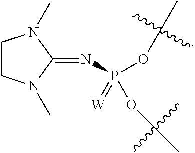

In certain embodiments, a non-negatively charged internucleotidic linkage has the structure of

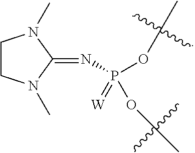

In some embodiments, a non-negatively charged internucleotidic linkage has the structure of

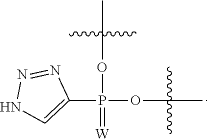

In some embodiments, a non-negatively charged internucleotidic linkage has the structure of

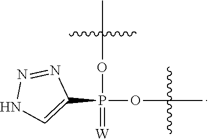

In some embodiments, a non-negatively charged internucleotidic linkage has the structure of

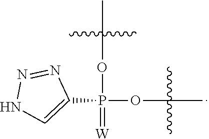

In some embodiments, a non-negatively charged internucleotidic linkage has the structure of

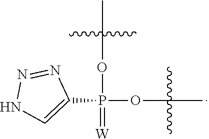

In some embodiments, a non-negatively charged internucleotidic linkage has the structure of

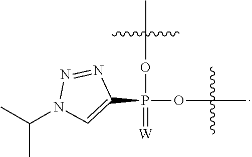

In some embodiments, anon-negatively charged internucleotidic linkage has the structure of

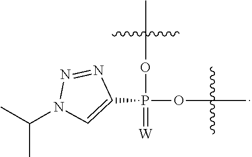

In some embodiments, a non-negatively charged internucleotidic linkage has the structure of

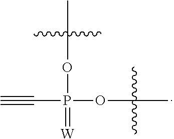

In some embodiments, a non-negatively charged internucleotidic linkage has the structure of

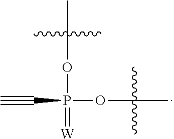

In some embodiments, a non-negatively charged internucleotidic linkage has the structure of

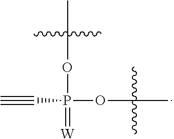

In some embodiments, W is O. In some embodiments, W is S. In some embodiments, a neutral internucleotidic linkage is a non-negatively charged internucleotidic linkage described above.

In certain embodiments, provided ds oligonucleotides comprise 1 or more internucleotidic linkages of Formula I, I-a, I-b, I-c, I-n-1, I-n-2, I-n-3, I-n-4, II, II-a-1, II-a-2, II-b-1, II-b-2, II-c-1, II-c-2, II-d-1, or II-d-2, which are described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019/032612, WO 2019/055951, WO 2019/075357, WO 2019/200185, WO 2019/217784, and/or WO 2019/032612,2607, WO2019032612, WO 2019/055951, WO 2019/075357, WO 2019/200185, WO 2019/217784, and/or WO 2019/032612, the Formula I, I-a, I-b, I-c, I-n-1, I-n-2, I-n-3, I-n-4, II, II-a-1, II-a-2, II-b-1, II-b-2, II-c-1, II-c-2, II-d-1, or II-d-2, or salt forms thereof, each of which are independently incorporated herein by reference.

In certain embodiments, a ds oligonucleotide comprises a neutral internucleotidic linkage and a chirally controlled internucleotidic linkage. In certain embodiments, a ds oligonucleotide comprises a neutral internucleotidic linkage and a chirally controlled internucleotidic linkage which is not the neutral internucleotidic linkage. In certain embodiments, a ds oligonucleotide comprises a neutral internucleotidic linkage and a chirally controlled phosphorothioate internucleotidic linkage. In certain embodiments, the present disclosure provides a ds oligonucleotide comprising one or more non-negatively charged internucleotidic linkages and one or more phosphorothioate internucleotidic linkages, wherein each phosphorothioate internucleotidic linkage in the oligonucleotide is independently a chirally controlled internucleotidic linkage. In certain embodiments, the present disclosure provides a ds oligonucleotide comprising one or more neutral internucleotidic linkages and one or more phosphorothioate internucleotidic linkage, wherein each phosphorothioate internucleotidic linkage in the ds oligonucleotide is independently a chirally controlled internucleotidic linkage. In certain embodiments, a ds oligonucleotide comprises at least 5, 6, 7, 8, 9, 10, 11, 12, 13, 14, 15, 16, 17, 18, 19, 20 or more chirally controlled phosphorothioate internucleotidic linkages. In certain embodiments, non-negatively charged internucleotidic linkage is chirally controlled. In certain embodiments, non-negatively charged internucleotidic linkage is not chirally controlled. In certain embodiments, a neutral internucleotidic linkage is chirally controlled. In certain embodiments, a neutral internucleotidic linkage is not chirally controlled.

Without wishing to be bound by any particular theory, the present disclosure notes that a neutral internucleotidic linkage can be more hydrophobic than a phosphorothioate internucleotidic linkage (PS), which can be more hydrophobic than a natural phosphate linkage (PO). Typically, unlike a PS or PO, a neutral internucleotidic linkage bears less charge. Without wishing to be bound by any particular theory, the present disclosure notes that incorporation of one or more neutral internucleotidic linkages into a ds oligonucleotide may increase the ds oligonucleotides' ability to be taken up by a cell and/or to escape from endosomes. Without wishing to be bound by any particular theory, the present disclosure notes that incorporation of one or more neutral internucleotidic linkages can be utilized to modulate melting temperature of duplexes formed between a ds oligonucleotide and its target nucleic acid.

Without wishing to be bound by any particular theory, the present disclosure notes that incorporation of one or more non-negatively charged internucleotidic linkages, e.g., neutral internucleotidic linkages, into a ds oligonucleotide may be able to increase the ds oligonucleotide's ability to mediate a function such as target adenosine editing.

As appreciated by those skilled in the art, internucleotidic linkages such as natural phosphate linkages and those of Formula I, I-a, I-b, I-c, I-n-1, I-n-2, I-n-3, I-n-4, II, II-a-1, II-a-2, II-b-1, II-b-2, II-c-1, II-c-2, II-d-1, II-d-2, or salt forms thereof typically connect two nucleosides (which can either be natural or modified) as described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019032612, WO 2019/055951, WO 2019/075357, WO 2019/200185, WO 2019/217784, and/or WO 2019/032612, the Formula I, I-a, I-b, I-c, I-n-1, I-n-2, I-n-3, I-n-4, II, II-a-1, II-a-2, II-b-1, II-b-2, II-c-1, II-c-2, II-d-1, II-d-2, or salt forms thereof, each of which are independently incorporated herein by reference. A typical connection, as in natural DNA and RNA, is that an internucleotidic linkage forms bonds with two sugars (which can be either unmodified or modified as described herein). In many embodiments, as exemplified herein an internucleotidic linkage forms bonds through its oxygen atoms or heteroatoms (e.g., Y and Z in various formulae) with one optionally modified ribose or deoxyribose at its 5′ carbon, and the other optionally modified ribose or deoxyribose at its 3′ carbon. In certain embodiments, each nucleoside units connected by an internucleotidic linkage independently comprises a nucleobase which is independently an optionally substituted A, T, C, G, or U, or a substituted tautomer of A, T, C, G or U, or a nucleobase comprising an optionally substituted heterocyclyl and/or a heteroaryl ring having at least one nitrogen atom.

In some embodiments, a linkage has the structure of or comprises Y—P^{L}(—X—R^{L})—Z—, or a salt form thereof, wherein:
P^{L} is P, P(═W), P->B(-L^{L}-R^{L})₃, or P^{N};
W is O, N(-L^{L}-R^{L}), S or Se;
P^{N} is P═N—C(-L^{L}-R′)(=L^{N}-R′) or P═N-L^{L}-R^{L};
L^{N} is ═N-L^{L1}-, ═CH-L^{L1}- wherein CH is optionally substituted, or ═N*(R′)(Q⁻)-L^{L1}-;
Q⁻ is an anion;
each of X, Y and Z is independently —O—, —S—, -L^{L}-N(-L^{L}-R^{L})-L^{L}-, -L^{L}-N═C(-L^{L}-R^{L})-L^{L}-, or L^{L};
each R^{L} is independently -L^{L}-N(R′)₂, -L^{L}-R′, —N═C(-L^{L}-R′)₂, -L^{L}-N(R′)C(NR′)N(R′)₂, -L^{L}-N(R′)C(O)N(R′)₂, a carbohydrate, or one or more additional chemical moieties optionally connected through a linker;
each of L^{L1} and L^{L} is independently L;
—Cy^{IL}- is -Cy-;
each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(NR′)N(R′)—, —N(R′)C(NR′)N(R′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, —OP(OR′)[B(R′)₃]O—, and —[C(R′)₂C(R′)₂O]n-, wherein n is 1-50, and one or more nitrogen or carbon atoms are optionally and independently replaced with Cy^{L};
each -Cy- is independently an optionally substituted bivalent 3-30 membered, monocyclic, bicyclic or polycyclic ring having 0-10 heteroatoms;
each Cy^{L} is independently an optionally substituted trivalent or tetravalent, 3-30 membered, monocyclic, bicyclic or polycyclic ring having 0-10 heteroatoms;
each R′ is independently —R, —C(O)R, —C(O)N(R)₂, —C(O)OR, or —S(O)₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, an internucleotidic linkage has the structure of —O—P^{L}(—X—R^{L})—O—, wherein each variable is independently as described herein. In some embodiments, an internucleotidic linkage has the structure of —O—P(═W)—X—R^{L})—O—, wherein each variable is independently as described herein. In some embodiments, an internucleotidic linkage has the structure of —O—P(═W)[—N(-L^{L}-R^{L})—R^{L}]—O—, wherein each variable is independently as described herein. In some embodiments, an internucleotidic linkage has the structure of —O—P(═W)(—NH-L^{L}-R^{L})—O—, wherein each variable is independently as described herein. In some embodiments, an internucleotidic linkage has the structure of —O—P(═W)[—N(R′)₂]—O—, wherein each variable is independently as described herein. In some embodiments, an internucleotidic linkage has the structure of —O—P(═W)(—NHR′)—O—, wherein each variable is independently as described herein. In some embodiments, an internucleotidic linkage has the structure of —O—P(═W)(—NHSO₂R)—O—, wherein each variable is independently as described herein. In some embodiments, an internucleotidic linkage has the structure of —O—P(═W)[—N═C(-L^{L}-R′)₂]—O—, wherein each variable is independently as described herein. In some embodiments, an internucleotidic linkage has the structure of —O—P(═W)[—N═C[N(R′)₂]₂]—O—, wherein each variable is independently as described herein. In some embodiments, an internucleotidic linkage has the structure of —OP(═W)(—N═C(R″)₂)—O—, wherein each variable is independently as described herein. In some embodiments, an internucleotidic linkage has the structure of —OP(═W)(—N(R″)₂)—O—, wherein each variable is independently as described herein. In some embodiments, W is O. In some embodiments, W is S. In some embodiments, such an internucleotidic linkage is a non-negatively charged internucleotidic linkage. In some embodiments, such an internucleotidic linkage is a neutral internucleotidic linkage.

In some embodiments, an internucleotidic linkage has the structure of —P^{L}(—X—R^{L})—Z—, wherein each variable is independently as described herein. In some embodiments, an internucleotidic linkage has the structure of —P^{L}(—X—R^{L})O—, wherein each variable is independently as described herein. In some embodiments, an internucleotidic linkage has the structure of —P(═W)(—X—R^{L})—O—, wherein each variable is independently as described herein. In some embodiments, an internucleotidic linkage has the structure of —P(═W)[—N(-L^{L}-R^{L})—R^{L}]—O—, wherein each variable is independently as described herein. In some embodiments, an internucleotidic linkage has the structure of —P(═W)(—NH-L^{L}-R^{L})—O—, wherein each variable is independently as described herein. In some embodiments, an internucleotidic linkage has the structure of —P(═W)[—N(R′)₂]—O—, wherein each variable is independently as described herein. In some embodiments, an internucleotidic linkage has the structure of —P(═W)(—NHR′)—O—, wherein each variable is independently as described herein. In some embodiments, an internucleotidic linkage has the structure of —P(═W)(—NHSO₂R)—O—, wherein each variable is independently as described herein. In some embodiments, an internucleotidic linkage has the structure of —P(═W)[—N═C(-L^{L}-R′)₂]—O—, wherein each variable is independently as described herein. In some embodiments, an internucleotidic linkage has the structure of —P(═W)[—N═C[N(R′)₂]₂]—O—, wherein each variable is independently as described herein. In some embodiments, an internucleotidic linkage has the structure of —P(═W)(—N═C(R″)₂)—O—, wherein each variable is independently as described herein. In some embodiments, an internucleotidic linkage has the structure of —P(═W)(—N(R″)₂)—O—, wherein each variable is independently as described herein. In some embodiments, W is O. In some embodiments, W is S. In some embodiments, such an internucleotidic linkage is a non-negatively charged internucleotidic linkage. In some embodiments, such an internucleotidic linkage is a neutral internucleotidic linkage. In some embodiments, P of such an internucleotidic linkage is bonded to N of a sugar.

In some embodiments, a linkage is a phosphoryl guanidine internucleotidic linkage. In some embodiments, a linkage is a thio-phosphoryl guanidine internucleotidic linkage.

In some embodiments, one or more methylene units are optionally and independently replaced with a moiety as described herein. In some embodiments, L or L^{L} is or comprises —SO₂—. In some embodiments, L or L^{L} is or comprises —SO₂N(R′)—. In some embodiments, L or L^{L} is or comprises —C(O)—. In some embodiments, L or L^{L} is or comprises —C(O)O—. In some embodiments, L or L^{L} is or comprises —C(O)N(R′)—. In some embodiments, L or L^{L} is or comprises —P(═W)(R′)—. In some embodiments, L or L^{L} is or comprises —P(═O)(R′)—. In some embodiments, L or L^{L} is or comprises —P(═S)(R′)—. In some embodiments, L or L^{L} is or comprises —P(R′)—. In some embodiments, L or L^{L} is or comprises —P(═W)(OR′)—. In some embodiments, L or L^{L} is or comprises —P(═O)(OR′)—. In some embodiments, L or L^{L} is or comprises —P(═S)(OR′)—. In some embodiments, L or L^{L} is or comprises —P(OR′)—. In some embodiments, —X—R^{L} is —N(R′)SO₂R^{L}. In some embodiments, —X—R^{L} is —N(R′)C(O)R^{L}. In some embodiments, —X—R^{L} is —N(R′)P(═O)(R′)R^{L}. In some embodiments, a linkage, e.g., a non-negatively charged internucleotidic linkage or neutral internucleotidic linkage, has the structure of or comprises —P(═W)(—N═C(R″)₂)—, —P(═W)(—N(R′)SO₂R″)—, —P(═W)(—N(R′)C(O)R″)—, —P(═W)(—N(R)₂)—, —P(═W)(—N(R′)P(O)(R″)₂)—, —OP(═W)(—N═C(R″)₂)O—, —OP(═W)(—N(R′)SO₂R″)O—, —OP(═W)(—N(R′)C(O)R″)O—, —OP(═W)(—N(R″)₂)O—, —OP(═W)(—N(R′)P(O)(R″)₂)O—, —P(═W)(—N═C(R″)₂)O—, —P(═W)(—N(R′)SO₂R″)O—, —P(═W)(—N(R′)C(O)R″)O—, —P(═W)(—N(R″)₂)O—, or —P(═W)(—N(R′)P(O)(R″)₂)O—, or a salt form thereof, wherein:
W is O or S;
each R″ is independently R′, —OR′, —P(═W)(R′)₂, or —N(R′)₂;
each R′ is independently —R, —C(O)R, —C(O)N(R)₂, —C(O)OR, or —S(O)₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, W is 0. In some embodiments, an internucleotidic linkage has the structure of —P(═O)(—N═C(R″)₂)—, —P(═O)(—N(R′)SO₂R″)—, —P(═O)(—N(R′)C(O)R″)—, —P(═O)(—N(R″)₂)—, —P(═O)(—N(R′)P(O)(R″)₂)—, —OP(═O)(—N═C(R″)₂)O—, —OP(═O)(—N(R′)SO₂R″)O—, —OP(═O)(—N(R′)C(O)R″)O—, —OP(═O)(—N(R″)₂)O—, —OP(═O)(—N(R′)P(O)(R″)₂)O—, —P(═O)(—N═C(R″)₂)O—, —P(═O)(—N(R′)SO₂R″)O—, —P(═O)(—N(R′)C(O)R″)O—, —P(═O)(—N(R″)₂)O—, or —P(═O)(—N(R′)P(O)(R″)₂)O—, or a salt form thereof. In some embodiments, an internucleotidic linkage has the structure of —P(═O)(—N═C(R″)₂)— —P(═O)—N(R″)₂)—, —OP(═O)(—N═C(R″)₂)—O—, —OP(═O)(—N(R″)₂)—O—, —P(═O)(—N═C(R″)₂)—O— or —P(═O)(—N(R″)₂)—O— or a salt form thereof. In some embodiments, an internucleotidic linkage has the structure of —OP(═O)(—N═C(R″)₂)—O— or —OP(═O)(—N(R″)₂)—O—, or a salt form thereof. In some embodiments, an internucleotidic linkage has the structure of —OP(═O)(—N═C(R″)₂)—O—, or a salt form thereof. In some embodiments, an internucleotidic linkage has the structure of —OP(═O)(—N(R″)₂)—O—, or a salt form thereof. In some embodiments, an internucleotidic linkage has the structure of —OP(═O)(—N(R′)SO₂R″)O—, or a salt form thereof. In some embodiments, an internucleotidic linkage has the structure of —OP(═O)(—N(R′)C(O)R″)O—, or a salt form thereof. In some embodiments, an internucleotidic linkage has the structure of —OP(═O)(—N(R′)P(O)(R″)₂)O—, or a salt form thereof. In some embodiments, a internucleotidic linkage is n001.

In some embodiments, W is S. In some embodiments, an internucleotidic linkage has the structure of —P(═S)(—N═C(R″)₂)—, —P(═S)(—N(R′)SO₂R″)—, —P(═S)(—N(R′)C(O)R″)—, —P(═S)—N(R″)₂)—, —P(═S)(—N(R′)P(O)(R″)₂)—, —OP(═S)(—N═C(R″)₂)O—, —OP(═S)(—N(R′)SO₂R″)O—, —OP(═S)(—N(R′)C(O)R″)O—, —OP(═S)(—N(R″)₂)O—, —OP(═S)(—N(R′)P(O)(R″)₂)O—, —P(═S)(—N═C(R″)₂)O—, —P(═S)(—N(R′)SO₂R″)O—, —P(═S)(—N(R′)C(O)R″)O—, —P(═S)(—N(R″)₂)O—, or —P(═S)(—N(R′)P(O)(R″)₂)O—, or a salt form thereof. In some embodiments, an internucleotidic linkage has the structure of —P(═S)(—N═C(R″)₂)— —P(═S)(—N(R″)₂)—, —OP(═S)(—N═C(R″)₂)—O—, —OP(═S)(—N(R″)₂)—O—, —P(═S)(—N═C(R″)₂)—O— or —P(═S)(—N(R″)₂)—O— or a salt form thereof. In some embodiments, an internucleotidic linkage has the structure of —OP(═S)(—N═C(R″)₂)—O— or —OP(═S)(—N(R″)₂)—O—, or a salt form thereof. In some embodiments, an internucleotidic linkage has the structure of —OP(═S)(—N═C(R″)₂)—O—, or a salt form thereof. In some embodiments, an internucleotidic linkage has the structure of —OP(═S)(—N(R″)₂)—O—, or a salt form thereof. In some embodiments, an internucleotidic linkage has the structure of —OP(═S)(—N(R′)SO₂R″)O—, or a salt form thereof. In some embodiments, an internucleotidic linkage has the structure of —OP(═S)(—N(R′)C(O)R″)O—, or a salt form thereof. In some embodiments, an internucleotidic linkage has the structure of —OP(═S)(—N(R′)P(O)(R″)₂)O—, or a salt form thereof. In some embodiments, a internucleotidic linkage is *n001.

In some embodiments, an internucleotidic linkage has the structure of —P(═O)(—N(R′)SO₂R″)—, wherein R″ is as described herein. In some embodiments, an internucleotidic linkage has the structure of —P(═S)(—N(R′)SO₂R″)—, wherein R″ is as described herein. In some embodiments, an internucleotidic linkage has the structure of —P(═O)(—N(R′)SO₂R″)O—, wherein R″ is as described herein. In some embodiments, an internucleotidic linkage has the structure of —P(═S)(—N(R′)SO₂R″)O—, wherein R″ is as described herein. In some embodiments, an internucleotidic linkage has the structure of —OP(═O)(—N(R′)SO₂R″)O—, wherein R″ is as described herein. In some embodiments, an internucleotidic linkage has the structure of —OP(═S)(—N(R′)SO₂R″)O—, wherein R″ is as described herein. In some embodiments, R′, e.g., of —N(R′)—, is hydrogen or optionally substituted C₁₋₆ aliphatic. In some embodiments, R′ is C₁₋₆ alkyl. In some embodiments, R′ is hydrogen. In some embodiments, R″, e.g., in —SO₂R″, is R′ as described herein. In some embodiments, an internucleotidic linkage has the structure of —P(═O)(—NHSO₂R″)—, wherein R″ is as described herein. In some embodiments, an internucleotidic linkage has the structure of —P(═S)(—NHSO₂R″)—, wherein R″ is as described herein. In some embodiments, an internucleotidic linkage has the structure of —P(═O)(—NHSO₂R″)O—, wherein R″ is as described herein. In some embodiments, an internucleotidic linkage has the structure of —P(═S)(—NHSO₂R″)O—, wherein R″ is as described herein. In some embodiments, an internucleotidic linkage has the structure of —OP(═O)(—NHSO₂R″)O—, wherein R″ is as described herein. In some embodiments, an internucleotidic linkage has the structure of —OP(═S)(—NHSO₂R″)O—, wherein R″ is as described herein. In some embodiments, —X—R^{L} is —N(R′)SO₂R^{L}, wherein each of R′ and R^{L} is independently as described herein. In some embodiments, R^{L} is R″. In some embodiments, R^{L} is R′. In some embodiments, —X—R^{L} is —N(R′)SO₂R″, wherein R′ is as described herein. In some embodiments, —X—R^{L} is —N(R′)SO₂R′, wherein R′ is as described herein. In some embodiments, —X—R^{L} is —NHSO₂R′, wherein R′ is as described herein. In some embodiments, R′ is R as described herein. In some embodiments, R′ is optionally substituted C₁₋₆ aliphatic. In some embodiments, R′ is optionally substituted C₁₋₆ alkyl. In some embodiments, R′ is optionally substituted phenyl. In some embodiments, R′ is optionally substituted heteroaryl. In some embodiments, R″, e.g., in —SO₂R″, is R. In some embodiments, R is an optionally substituted group selected from C₁₋₆ aliphatic, aryl, heterocyclyl, and heteroaryl. In some embodiments, R is optionally substituted C₁₋₆ aliphatic. In some embodiments, R is optionally substituted C₁₋₆ alkyl. In some embodiments, R is optionally substituted C₁₋₆ alkenyl. In some embodiments, R is optionally substituted C₁₋₆ alkynyl. In some embodiments, R is optionally substituted methyl. In some embodiments, —X—R^{L} is —NHSO₂CH₃. In some embodiments, R is —CF₃. In some embodiments, R is methyl. In some embodiments, R is optionally substituted ethyl. In some embodiments, R is ethyl. In some embodiments, R is —CH₂CHF₂. In some embodiments, R is —CH₂CH₂OCH₃. In some embodiments, R is optionally substituted propyl. In some embodiments, R is optionally substituted butyl. In some embodiments, R is n-butyl. In some embodiments, R is —(CH₂)₆NH₂. In some embodiments, R is an optionally substituted linear C₂₋₂₀ aliphatic. In some embodiments, R is optionally substituted linear C₂₋₂₀ alkyl. In some embodiments, R is linear C₂₋₂₀ alkyl. In some embodiments, R is optionally substituted C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ aliphatic. In some embodiments, R is optionally substituted C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is optionally substituted linear C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is linear C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is optionally substituted phenyl. In some embodiments, R is phenyl. In some embodiments, R is p-methylphenyl. In some embodiments, R is 4-dimethylaminophenyl. In some embodiments, R is 3-pyridinyl. In some embodiments, R is

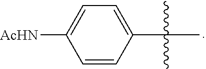 [SMILES: *c1ccc(NC(C)=O)cc1]

In some embodiments, R is

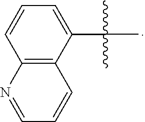 [SMILES: *c1cccc2ncccc12]

In some embodiments, R is benzyl. In some embodiments, R is optionally substituted heteroaryl. In some embodiments, R is optionally substituted 1,3-diazolyl. In some embodiments, R is optionally substituted 2-(1,3)-diazolyl. In some embodiments, R is optionally substituted 1-methyl-2-(1,3)-diazolyl. In some embodiments, R is isopropyl. In some embodiments, R″ is —N(R′)₂. In some embodiments, R″ is —N(CH₃)₂. In some embodiments, R″, e.g., in —SO₂R″, is —OR′, wherein R′ is as described herein. In some embodiments, R′ is R as described herein. In some embodiments, R″ is —OCH₃. In some embodiments, a linkage is —OP(═O)(—NHSO₂R)O—, wherein R is as described herein. In some embodiments, R is optionally substituted linear alkyl as described herein. In some embodiments, R is linear alkyl as described herein. In some embodiments, a linkage is —OP(═O)(—NHSO₂CH₃)O—. In some embodiments, a linkage is —OP(═O)(—NHSO₂CH₂CH₃)O—. In some embodiments, a linkage is —OP(═O)(—NHSO₂CH₂CH₂OCH₃)O—. In some embodiments, a linkage is —OP(═O)(—NHSO₂CH₂Ph)O—. In some embodiments, a linkage is —OP(═O)(—NHSO₂CH₂CHF₂)O—. In some embodiments, a linkage is —OP(═O)(—NHSO₂(4-methylphenyl))O—. In some embodiments, —X—R^{L} is

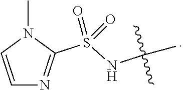 [SMILES: *NS(=O)(=O)c1nccn1C]

In some embodiments, a linkage is —OP(═O)(—X—R^{L})O—, wherein —X—R^{L} is

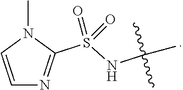 [SMILES: *NS(=O)(=O)c1nccn1C]

In some embodiments, a linkage is —OP(═O)(—NHSO₂CH(CH₃)₂)O—. In some embodiments, a linkage is —OP(═O)(—NHSO₂N(CH₃)₂)O—.

In some embodiments, an internucleotidic linkage has the structure of —P(═O)(—N(R′)C(O)R″)—, wherein R″ is as described herein. In some embodiments, an internucleotidic linkage has the structure of —P(═S)(—N(R′)C(O)R″)—, wherein R″ is as described herein. In some embodiments, an internucleotidic linkage has the structure of —P(═O)(—N(R′)C(O)R″)O—, wherein R″ is as described herein. In some embodiments, an internucleotidic linkage has the structure of —P(═S)(—N(R′)C(O)R″)O—, wherein R″ is as described herein. In some embodiments, an internucleotidic linkage has the structure of —OP(═O)(—N(R′)C(O)R″)O—, wherein R″ is as described herein. In some embodiments, an internucleotidic linkage has the structure of —OP(═S)(—N(R′)C(O)R″)O—, wherein R″ is as described herein. In some embodiments, R′, e.g., of —N(R′)—, is hydrogen or optionally substituted C₁₋₆ aliphatic. In some embodiments, R′ is C₁₋₆ alkyl. In some embodiments, R′ is hydrogen. In some embodiments, R″, e.g., in —C(O)R″, is R′ as described herein. In some embodiments, an internucleotidic linkage has the structure of —P(═O)(—NHC(O)R″)—, wherein R″ is as described herein. In some embodiments, an internucleotidic linkage has the structure of —P(═S)(—NHC(O)R″)—, wherein R″ is as described herein. In some embodiments, an internucleotidic linkage has the structure of —P(═O)(—NHC(O)R″)O—, wherein R″ is as described herein. In some embodiments, an internucleotidic linkage has the structure of —P(═S)(—NHC(O)R″)O—, wherein R″ is as described herein. In some embodiments, an internucleotidic linkage has the structure of —OP(═O)(—NHC(O)R″)O—, wherein R″ is as described herein. In some embodiments, an internucleotidic linkage has the structure of —OP(═S)(—NHC(O)R″)O—, wherein R″ is as described herein. In some embodiments, —X—R^{L}is —N(R′)COR^{L}, wherein R^{L} is as described herein. In some embodiments, —X—R^{L} is —N(R′)COR″, wherein R″ is as described herein. In some embodiments, —X—R^{L} is —N(R′)COR′, wherein R′ is as described herein. In some embodiments, —X—R^{L} is —NHCOR′, wherein R′ is as described herein. In some embodiments, R′ is R as described herein. In some embodiments, R′ is optionally substituted C₁₋₆ aliphatic. In some embodiments, R′ is optionally substituted C₁₋₆ alkyl. In some embodiments, R′ is optionally substituted phenyl. In some embodiments, R′ is optionally substituted heteroaryl. In some embodiments, R″, e.g., in —C(O)R″, is R. In some embodiments, R is an optionally substituted group selected from C₁₋₆ aliphatic, aryl, heterocyclyl, and heteroaryl. In some embodiments, R is optionally substituted C₁₋₆ aliphatic. In some embodiments, R is optionally substituted C₁₋₆ alkyl. In some embodiments, R is optionally substituted C₁₋₆ alkenyl. In some embodiments, R is optionally substituted C₁₋₆ alkynyl. In some embodiments, R is methyl. In some embodiments, —X—R^{L} is —NHC(O)CH₃. In some embodiments, R is optionally substituted methyl. In some embodiments, R is —CF₃. In some embodiments, R is optionally substituted ethyl. In some embodiments, R is ethyl. In some embodiments, R is —CH₂CHF₂. In some embodiments, R is —CH₂CH₂OCH₃. In some embodiments, R is optionally substituted C₁₋₂₀ (e.g., C₁₋₆, C₂₋₆, C₃₋₆, C₁₋₁₀, C₂₋₁₀, C₃₋₁₀, C₂-2₀, C₃₋₂₀, C₁₀₋₂₀, 1, 2, 3, 4, 5, 6, 7, 8, 9, 10, 11, 12, 13, 14, 15, 16, 17, 18, 19, or 20, etc.) aliphatic. In some embodiments, R is optionally substituted C₁₋₂₀ (e.g., C₁₋₆, C₂₋₆, C₃₋₆, C₁₋₁₀, C₂₋₁₀, C₃₋₁₀, C₂₋₂₀, C₃₋₂₀, C₁₀₋₂₀, 1, 2, 3, 4, 5, 6, 7, 8, 9, 10, 11, 12, 13, 14, 15, 16, 17, 18, 19, or 20, etc.) alkyl. In some embodiments, R is an optionally substituted linear C₂₋₂₀ aliphatic. In some embodiments, R is optionally substituted linear C₂₋₂₀ alkyl. In some embodiments, R is linear C₂₋₂₀ alkyl. In some embodiments, R is optionally substituted C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ aliphatic. In some embodiments, R is optionally substituted C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is optionally substituted linear C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is linear C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is optionally substituted aryl. In some embodiments, R is optionally substituted phenyl. In some embodiments, R is p-methylphenyl. In some embodiments, R is benzyl. In some embodiments, R is optionally substituted heteroaryl. In some embodiments, R is optionally substituted 1,3-diazolyl. In some embodiments, R is optionally substituted 2-(1,3)-diazolyl. In some embodiments, R is optionally substituted 1-methyl-2-(1,3)-diazolyl. In some embodiments, R^{L} is —(CH₂)₅NH₂. In some embodiments, R^{L} is

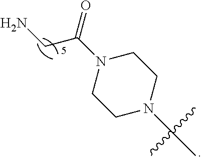

In some embodiments, R^{L} is

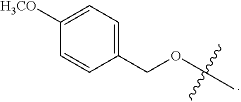 [SMILES: *OCc1ccc(OC)cc1]

In some embodiments, R″ is —N(R′)₂. In some embodiments, R″ is —N(CH₃)₂. In some embodiments, —X—R^{L} is —N(R′)CON(R^{L})₂, wherein each of R′ and R^{L} is independently as described herein. In some embodiments, —X—R^{L} is —NHCON(R^{L})₂, wherein R^{L} is as described herein. In some embodiments, two R′ or two R^{L} are taken together with the nitrogen atom to which they are attached to form a ring as described herein, e.g., optionally substituted

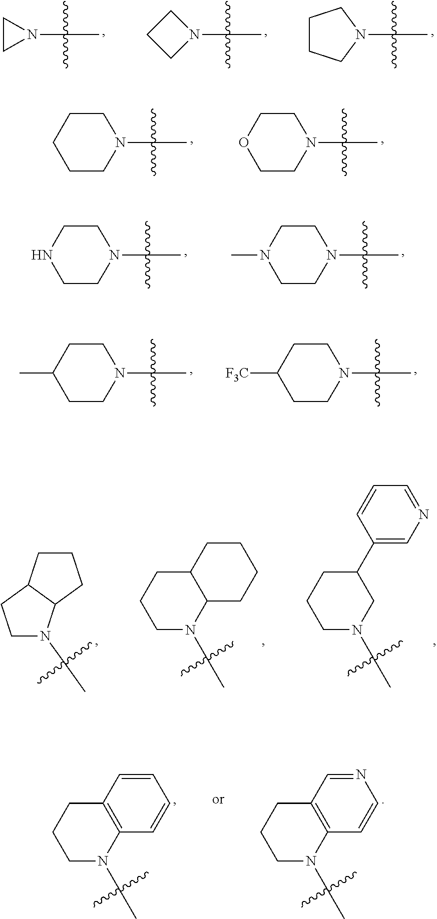

In some embodiments, R″, e.g., in —C(O)R″, is —OR′, wherein R′ is as described herein. In some embodiments, R′ is R as described herein. In some embodiments, is optionally substituted C₁₋₆ aliphatic. In some embodiments, is optionally substituted C₁₋₆ alkyl. In some embodiments, R″ is —OCH₃. In some embodiments, —X—R^{L} is —N(R′)C(O)OR^{L}, wherein each of R′ and R^{L} is independently as described herein. In some embodiments, R is

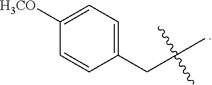 [SMILES: *Cc1ccc(OC)cc1]

In some embodiments, —X—R^{L} is —NHC(O)OCH₃. In some embodiments, —X—R^{L} is —NHC(O)N(CH₃)₂. In some embodiments, a linkage is —OP(O)(NHC(O)CH₃)O—. In some embodiments, a linkage is —OP(O)(NHC(O)OCH₃)O—. In some embodiments, a linkage is —OP(O)(NHC(O)(p-methylphenyl))O—. In some embodiments, a linkage is —OP(O)(NHC(O)N(CH₃)₂)O—. In some embodiments, —X—R^{L} is —N(R′)R^{L}, wherein each of R′ and R^{L} is independently as described herein. In some embodiments, —X—R^{L} is —N(R′)R^{L}, wherein each of R′ and R^{L} is independently not hydrogen. In some embodiments, —X—R^{L} is —NHR^{L}, wherein R^{L} is as described herein. In some embodiments, R^{L} is not hydrogen. In some embodiments, R^{L} is optionally substituted aryl or heteroaryl. In some embodiments, R^{L} is optionally substituted aryl. In some embodiments, R^{L} is optionally substituted phenyl. In some embodiments, —X—R^{L} is —N(R′)₂, wherein each R′ is independently as described herein. In some embodiments, —X—R^{L} is —NHR′, wherein R′ is as described herein. In some embodiments, —X—R^{L} is —NHR, wherein R is as described herein. In some embodiments, —X—R^{L} is R^{L}, wherein R^{L} is as described herein. In some embodiments, R^{L} is —N(R′)₂, wherein each R′ is independently as described herein. In some embodiments, R^{L} is —NHR′, wherein R′ is as described herein. In some embodiments, R^{L} is —NHR, wherein R is as described herein. In some embodiments, R^{L} is —N(R′)₂, wherein each R′ is independently as described herein. In some embodiments, none of R′ in —N(R′)₂ is hydrogen. In some embodiments, R^{L} is —N(R′)₂, wherein each R′ is independently C₁₋₆ aliphatic. In some embodiments, R^{L} is -L-R′, wherein each of L and R′ is independently as described herein. In some embodiments, R^{L} is -L-R, wherein each of L and R is independently as described herein. In some embodiments, R^{L} is —N(R′)—Cy-N(R′)—R′. In some embodiments, R^{L} is —N(R′)—Cy-C(O)—R′. In some embodiments, R^{L} is —N(R′)—Cy-O—R′. In some embodiments, R^{L} is —N(R′)—Cy-SO₂—R′. In some embodiments, R^{L} is —N(R′)—Cy-SO₂—N(R′)₂. In some embodiments, R^{L} is —N(R′)—Cy-C(O)—N(R′)₂. In some embodiments, R^{L} is —N(R′) -Cy-OP(O)(R″)₂. In some embodiments, -Cy- is an optionally substituted bivalent aryl group. In some embodiments, -Cy- is optionally substituted phenylene. In some embodiments, -Cy- is optionally substituted 1,4-phenylene. In some embodiments, -Cy- is 1,4-phenylene. In some embodiments, R^{L} is —N(CH₃)₂. In some embodiments, R^{L} is —N(i-Pr)₂. In some embodiments, R^{L} is

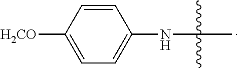

In some embodiments, R^{L} is

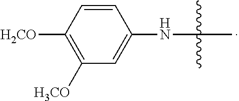

In some embodiments, R^{L} is

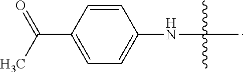 [SMILES: *Nc1ccc(C(C)=O)cc1]

In some embodiments, R^{L} is

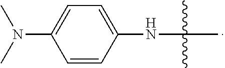 [SMILES: *Nc1ccc(N(C)C)cc1]

In some embodiments, R^{L} is

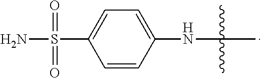 [SMILES: *Nc1ccc(S(N)(=O)=O)cc1]

In some embodiments, R^{L} is

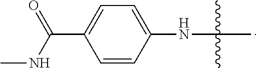 [SMILES: *Nc1ccc(C(=O)NC)cc1]

In some embodiments, R^{L} is

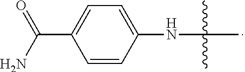 [SMILES: *Nc1ccc(C(N)=O)cc1]

In some embodiments, R^{L} is

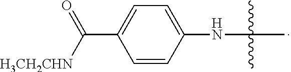

In some embodiments, R^{L} is

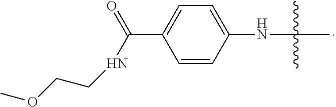 [SMILES: *Nc1ccc(C(=O)NCCOC)cc1]

In some embodiments, R^{L} is

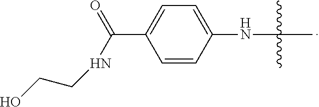 [SMILES: *Nc1ccc(C(=O)NCCO)cc1]

In some embodiments, R^{L} is

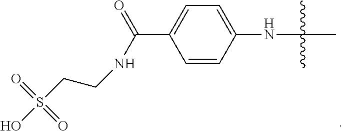 [SMILES: *Nc1ccc(C(=O)NCCS(=O)(=O)O)cc1]

In some embodiments, R^{L} is

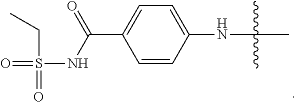 [SMILES: *Nc1ccc(C(=O)NS(=O)(=O)CC)cc1]

In some embodiments, R^{L} is

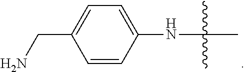 [SMILES: *Nc1ccc(CN)cc1]

In some embodiments, R^{L} is

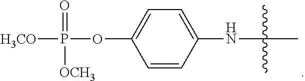 [SMILES: *Nc1ccc(OP(=O)(OC)OC)cc1]

In some embodiments, R^{L} is

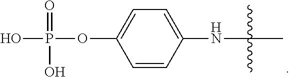 [SMILES: *Nc1ccc(OP(=O)(O)O)cc1]

In some embodiments, R^{L} is

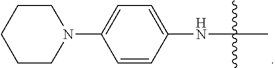 [SMILES: *Nc1ccc(N2CCCCC2)cc1]

In some embodiments, R^{L} is

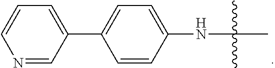 [SMILES: *Nc1ccc(-c2cccnc2)cc1]

In some embodiments, R^{L} is

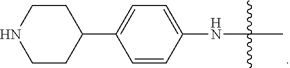 [SMILES: *Nc1ccc(C2CCNCC2)cc1]

In some embodiments, R^{L} is

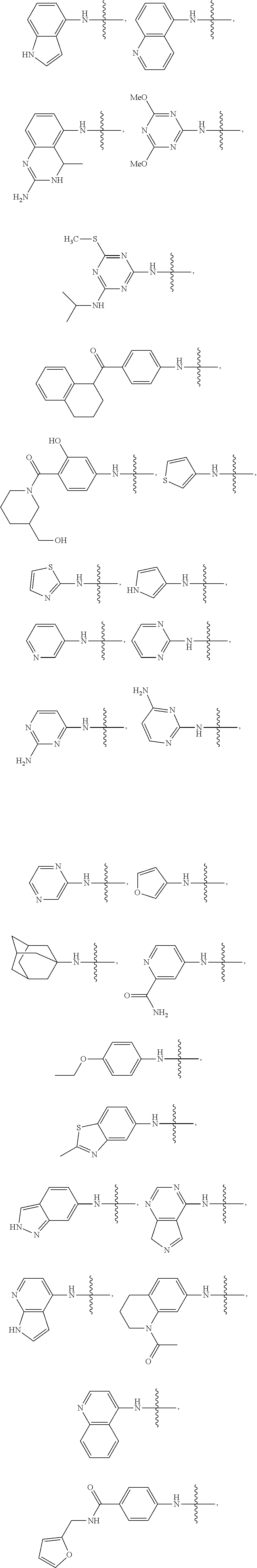

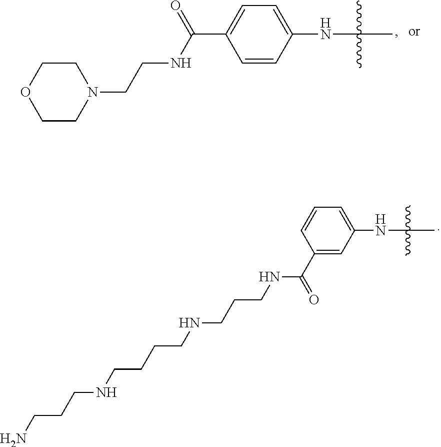 [SMILES: *Nc1ccc(C(=O)NCCN2CCOCC2)cc1 ; *Nc1cccc(C(=O)NCCCNCCCCNCCCN)c1]

In some embodiments, —X—R^{L} is —N(R′)—C(O)-Cy-R^{L} In some embodiments, —X—R^{L} is R^{L}. In some embodiments, R^{L} is —N(R′)—C(O)-Cy-O—R′. In some embodiments, R^{L} is —N(R′)—C(O)-Cy-R′. In some embodiments, R^{L} is —N(R′)—C(O) -Cy-C(O)—R′. In some embodiments, R^{L} is —N(R′)—C(O)-Cy-N(R′)₂. In some embodiments, R^{L} is —N(R′)—C(O)-Cy-SO₂—N(R′)₂. In some embodiments, R^{L} is —N(R′)—C(O)-Cy-C(O)—N(R′)₂. In some embodiments, R^{L} is —N(R′)—C(O)-Cy-C(O)—N(R′)—SO₂—R′. In some embodiments, R′ is R as described herein. In some embodiments, R^{L} is

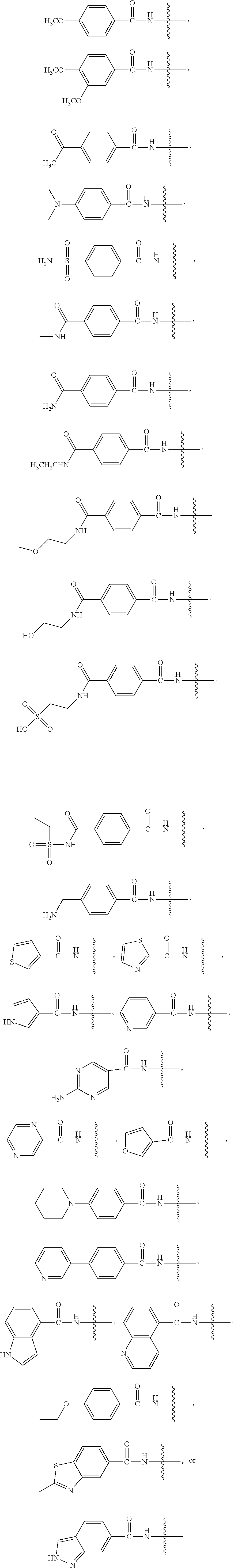

As described herein, in some embodiments, one or more methylene units of L, or a variable which comprises or is L, are independently replaced with —O—, —N(R′)—, —(O)—, —C(O)N(R′)—, —SO₂—, —SO₂N(R′)—, or -Cy-. In some embodiments, a methylene unit is replaced with -Cy-. In some embodiments, -Cy- is an optionally substituted bivalent aryl group. In some embodiments, -Cy- is optionally substituted phenylene. In some embodiments, -Cy- is optionally substituted 1,4-phenylene. In some embodiments, -Cy- is an optionally substituted bivalent 5-20 (e.g. 5, 6, 7, 8, 9, 10, 11, 12, 13, 14, 15, 16, 17, 18, 19, or 20) membered heteroaryl group having 1-10 (e.g., 1, 2, 3, 4, 5, 6, 7, 8, 9, or 10) heteroatoms. In some embodiments, -Cy- is monocyclic. In some embodiments, -Cy- is bicyclic. In some embodiments, -Cy- is polycyclic. In some embodiments, each monocyclic unit in -Cy- is independently 3-10 (e.g., 3, 4, 5, 6, 7, 8, 9, or 10) membered, and is independently saturated, partially saturated, or aromatic. In some embodiments, -Cy- is an optionally substituted 3-20 (e.g., 3, 4, 5, 6, 7, 8, 9, 10, 11, 12, 13, 14, 15, 16, 17, 18, 19, or 20) membered monocyclic, bicyclic or polycyclic aliphatic group. In some embodiments, -Cy- is an optionally substituted 3-20 (e.g., 3,4, 5,6, 7, 8,9, 10, 11, 12, 13, 14, 15, 16, 17, 18, 19, or 20) membered monocyclic, bicyclic or polycyclic heteroaliphatic group having 1-10 (e.g., 1, 2, 3, 4, 5, 6, 7, 8, 9, or 10) heteroatoms.

In some embodiments, an internucleotidic linkage has the structure of —P(═O)(—N(R′)P(O)R″)₂)—, wherein each R″ is independently as described herein. In some embodiments, an internucleotidic linkage has the structure of —P(═S)(—N(R′)P(O)(R″)₂)—, wherein each R″ is independently as described herein. In some embodiments, an internucleotidic linkage has the structure of —P(═O)(—N(R′)P(O)(R″)₂)O—, wherein each R″ is independently as described herein. In some embodiments, an internucleotidic linkage has the structure of —P(═S)(—N(R′)P(O)(R″)₂)O—, wherein each R″ is independently as described herein. In some embodiments, an internucleotidic linkage has the structure of —OP(═O)(—N(R′)P(O)(R″)₂)O—, wherein each R″ is independently as described herein. In some embodiments, an internucleotidic linkage has the structure of —OP(═S)(—N(R′)P(O)(R″)₂)O—, wherein each R″ is independently as described herein. In some embodiments, R′, e.g., of —N(R′)—, is hydrogen or optionally substituted C₁₋₆ aliphatic. In some embodiments, R′ is C₁₋₆ alkyl. In some embodiments, R′ is hydrogen. In some embodiments, R″, e.g., in —P(O)(R″)₂, is R′ as described herein. In some embodiments, an internucleotidic linkage has the structure of —P(═O)(—NHP(O)(R″)₂)—, wherein each R″ is independently as described herein. In some embodiments, an internucleotidic linkage has the structure of —P(═S)(—NHP(O)(R″)₂)—, wherein each R″ is independently as described herein. In some embodiments, an internucleotidic linkage has the structure of —P(═O)(—NHP(O)(R″)₂)O—, wherein each R″ is independently as described herein. In some embodiments, an internucleotidic linkage has the structure of —P(═S)(—NHP(O)(R″)₂)O—, wherein each R″ is independently as described herein. In some embodiments, an internucleotidic linkage has the structure of —OP(═O)(—NHP(O)(R″)₂)O—, wherein each R″ is independently as described herein. In some embodiments, an internucleotidic linkage has the structure of —OP(═S)(—NHP(O)(R″)₂)O—, wherein each R″ is independently as described herein. In some embodiments, an occurrence of R″, e.g., in —P(O)(R″)₂, is R. In some embodiments, R is an optionally substituted group selected from C₁₋₆ aliphatic, aryl, heterocyclyl, and heteroaryl. In some embodiments, R is optionally substituted C₁₋₆ aliphatic. In some embodiments, R is optionally substituted C₁₋₆ alkyl. In some embodiments, R is optionally substituted C₁₋₆ alkenyl. In some embodiments, R is optionally substituted C₁₋₆ alkynyl. In some embodiments, R is methyl. In some embodiments, R is optionally substituted methyl. In some embodiments, R is —CF₃. In some embodiments, R is optionally substituted ethyl. In some embodiments, R is ethyl. In some embodiments, R is —CH₂CHF₂. In some embodiments, R is —CH₂CH₂OCH₃. In some embodiments, R is optionally substituted C₁₋₂₀ (e.g., C₁—, C₂₋₆, C₃₋₆, C₁₋₁₀, C₂₋₁₀, C₃₋₁₀, C₂₋₂₀, C₃₋₂₀, C₁₀₋₂₀, 1, 2, 3, 4, 5, 6, 7, 8, 9, 10, 11, 12, 13, 14, 15, 16, 17, 18, 19, or 20, etc.) aliphatic. In some embodiments, R is optionally substituted C₁₋₂₀ (e.g., C₁₋₆, C₂₋₆, C₃₋₆, C₁₋₁₀, C₂₋₁₀, C₃₋₁₀, C₂₋₂₀, C₃₋₂₀, C₁₀₋₂₀, 1, 2, 3, 4, 5, 6, 7, 8, 9, 10, 11, 12, 13, 14, 15, 16, 17, 18, 19, or 20, etc.) alkyl. In some embodiments, R is an optionally substituted linear C₂₋₂₀ aliphatic. In some embodiments, R is optionally substituted linear C₂-20 alkyl. In some embodiments, R is linear C₂₋₂₀ alkyl. In some embodiments, R is isopropyl. In some embodiments, R is optionally substituted C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ aliphatic. In some embodiments, R is optionally substituted C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is optionally substituted linear C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀ alkyl. In some embodiments, R is linear C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂ alkyl. In some embodiments, each R″ is independently R as described herein, for example, in some embodiments, each R″ is methyl. In some embodiments, R″ is optionally substituted aryl. In some embodiments, R is optionally substituted phenyl. In some embodiments, R is p-methylphenyl. In some embodiments, R is benzyl. In some embodiments, R is optionally substituted heteroaryl. In some embodiments, R is optionally substituted 1,3-diazolyl. In some embodiments, R is optionally substituted 2-(1,3)-diazolyl. In some embodiments, R is optionally substituted 1-methyl-2-(1,3)-diazolyl. In some embodiments, an occurrence of R″ is —N(R′)₂. In some embodiments, R″ is —N(CH₃)₂. In some embodiments, an occurrence of R″, e.g., in —P(O)(R″)₂, is —OR′, wherein R′ is as described herein. In some embodiments, R′ is R as described herein. In some embodiments, is optionally substituted C₁₋₆ aliphatic. In some embodiments, is optionally substituted C₁₋₆ alkyl. In some embodiments, R″ is —OCH₃. In some embodiments, each R″ is —OR′ as described herein. In some embodiments, each R″ is —OCH₃. In some embodiments, each R″ is —OH. In some embodiments, a linkage is —OP(O)(NHP(O)(OH)₂)O—. In some embodiments, a linkage is —OP(O)(NHP(O)(OCH₃)₂)O—. In some embodiments, a linkage is —OP(O)(NHP(O)(CH₃)O—.

In some embodiments, —N(R″)₂ is —N(R′)₂. In some embodiments, —N(R″)₂ is —NHR. In some embodiments, —N(R″)₂ is —NHC(O)R. In some embodiments, —N(R″)₂ is —NHC(O)OR. In some embodiments, —N(R″)₂ is —NHS(O)₂R In some embodiments, an internucleotidic linkage is a phosphoryl guanidine internucleotidic linkage. In some embodiments, an internucleotidic linkage comprises —X—R^{L} as described herein. In some embodiments, —X—R^{L} is —N═C(-L^{L}-R^{L}) In some embodiments, —X—R^{L} is —N═C[N(R^{L})₂]₂. In some embodiments, —X—R^{L} is —N═C[NR′R^{L}]₂. In some embodiments, —X—R^{L} is —N═C[N(R′)₂]₂. In some embodiments, —X—R^{L} is —N═C[N(R^{L})₂](CHR^{L1}R^{L2}), wherein each of R^{L} and R^{L2} is independently as described herein. In some embodiments, —X—R^{L} is —N═C(NR′R^{L})(CHR^{L1}R^{L2}), wherein each of R^{L1} and R^{L2} is independently as described herein. In some embodiments, —X—R^{L} is —N═C(NR′R^{L})(CR′R^{L1}R^{L2}), wherein each of R^{L1} and R^{L2} is independently as described herein. In some embodiments, —X—R^{L} is —N═C[N(R′)₂](CHR′R^{L2}). In some embodiments, —X—R^{L} is —N═C[N(R^{L})₂](R^{L}). In some embodiments, —X—R^{L} is —N═C(NR′R^{L})(R^{L}). In some embodiments, —X—R^{L} is —N═C(NR′R^{L})(R′). In some embodiments, —X—R^{L} is —N═C[N(R′)₂](R′). In some embodiments, —X—R^{L} is —N═C(NR′R^{L1})(NR′R^{L2}), wherein each R^{L1} and R^{L2} is independently R^{L}, and each R′ and R^{L} is independently as described herein. In some embodiments, —X—R^{L} is —N═C(NR′R^{L1})(NR′R^{L2}), wherein variable is independently as described herein. In some embodiments, —X—R^{L} is —N═C(NR′R^{L1})(CHR′R^{L2}), wherein variable is independently as described herein. In some embodiments, —X—R^{L} is —N═C(NR′R^{L1})(R′), wherein variable is independently as described herein. In some embodiments, each R′ is independently R. In some embodiments, R is optionally substituted C₁₋₆ aliphatic. In some embodiments, R is methyl. In some embodiments, —X—R^{L} is

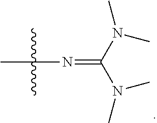 [SMILES: *N=C(N(C)C)N(C)C]

In some embodiments, two groups selected from R′, R^{L}, R^{L1}, R^{L2}, etc. (in some embodiments, on the same atom (e.g., —N(R′)₂, or —NR′R^{L}, or —N(R^{L})², wherein R′ and R^{L} can independently be R as described herein), etc.), or on different atoms (e.g., the two R′ in —N═C(NR′R^{L})(CR′R^{L1}R^{L2}) or —N═C(NR′R^{L1})(NR′R^{L2}); can also be two other variables that can be R, e.g., R^{L}, R^{L1}, R^{L2}, etc.)) are independently R and are taken together with their intervening atoms to form a ring as described herein. In some embodiments, two of R, R′, R^{L}, R^{L1}, or R^{L2} on the same atom, e.g., of —N(R′)₂, —N(R^{L})₂, —NR′R^{L}, —NR′R^{L1}, —NR′R^{L2}, —CR′R^{L1}R^{L2}, etc., are taken together to form a ring as described herein. In some embodiments, two R′, R^{L}, R^{L1}, or R^{L2} on two different atoms, e.g., the two R′ in —N═C(NR′R^{L})(CR′R^{L1}R^{L2}), —N═C(NR′R^{L1})(NR′R^{L2}), etc. are taken together to form a ring as described herein. In some embodiments, a formed ring is an optionally substituted 3-20 (e.g., 3-15, 3-12, 3-10, 3-9, 3-8, 3-7, 3-6, 4-15, 4-12, 4-10, 4-9, 4-8, 4-7, 4-6, 5-15, 5-12, 5-10, 5-9, 5-8, 5-7, 5-6, 1, 2, 3, 4, 5, 6, 7, 8, 9, 10, 11, 12, 13, 14, 15, 16, 17, 18, 19, 20, etc.) monocyclic, bicyclic or tricyclic ring having 0-5 additional heteroatoms. In some embodiments, a formed ring is monocyclic as described herein. In some embodiments, a formed ring is an optionally substituted 5-10 membered monocyclic ring. In some embodiments, a formed ring is bicyclic. In some embodiments, a formed ring is polycyclic. In some embodiments, two groups that are or can be R (e.g., the two R′ in —N═C(NR′R^{L})(CR′R^{L1}R^{L2}) or —N═C(NR′R^{L1})(NR′R^{L2}), the two R′ in —N═C(NR′R^{L})(CR′R^{L1}R^{L2}), —N═C(NR′R^{L1})(NR′R^{L2}), etc.) are taken together to form an optionally substituted bivalent hydrocarbon chain, e.g., an optionally substituted C₁₋₂₀ aliphatic chain, optionally substituted —(CH₂)n- wherein n is 1-20 (e.g., 1, 2, 3, 4, 5, 6, 7, 8, 9, 10, 11, 12, 13, 14, 15, 16, 17, 18, 19, or 20). In some embodiments, a hydrocarbon chain is saturated. In some embodiments, a hydrocarbon chain is partially unsaturated. In some embodiments, a hydrocarbon chain is unsaturated. In some embodiments, two groups that are or can be R (e.g., the two R′ in —N═C(NR′R^{L})(CR′R^{L1}R^{L2}) or —N═C(NR′R^{L1})(NR′R^{L2}), the two R′ in —N═C(NR′R^{L})(CR′R^{L1}R^{L2}), —N═C(NR′R^{L1})(NR′R^{L2}), etc.) are taken together to form an optionally substituted bivalent heteroaliphatic chain, e.g., an optionally substituted C₁₋₂₀ heteroaliphatic chain having 1-10 heteroatoms. In some embodiments, a heteroaliphatic chain is saturated. In some embodiments, a heteroaliphatic chain is partially unsaturated. In some embodiments, a heteroaliphatic chain is unsaturated. In some embodiments, a chain is optionally substituted —(CH₂)—. In some embodiments, a chain is optionally substituted —(CH₂)₂—. In some embodiments, a chain is optionally substituted —(CH₂)—. In some embodiments, a chain is optionally substituted —(CH₂)₂—. In some embodiments, a chain is optionally substituted —(CH₂)₃—. In some embodiments, a chain is optionally substituted —(CH₂)₄—. In some embodiments, a chain is optionally substituted —(CH₂)₅—. In some embodiments, a chain is optionally substituted —(CH₂)₆—. In some embodiments, a chain is optionally substituted —CH═CH—. In some embodiments, a chain is optionally substituted

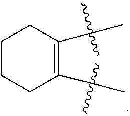 [SMILES: *C1=C(*)CCCC1]

In some embodiments, a chain is optionally substituted

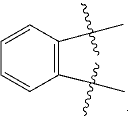 [SMILES: *c1ccccc1*]

In some embodiments, a chain is optionally substituted

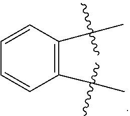 [SMILES: *c1ccccc1*]

In some embodiments, a chain is optionally substituted

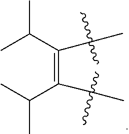 [SMILES: */C(=C(\*)C(C)C)C(C)C]

In some embodiments, a chain is optionally substituted

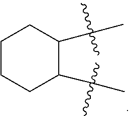 [SMILES: *C1CCCCC1*]

In some embodiments, a chain is optionally substituted

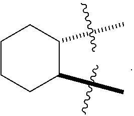 [SMILES: *[C@H]1CCCC[C@@H]1*]

In some embodiments, a chain is optionally substituted

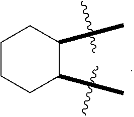 [SMILES: *[C@@H]1CCCC[C@@H]1*]

In some embodiments, a chain is optionally substituted

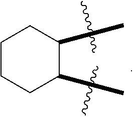 [SMILES: *[C@@H]1CCCC[C@@H]1*]

In some embodiments, a chain is optionally substituted

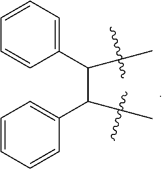 [SMILES: *C(c1ccccc1)C(*)c1ccccc1]

In some embodiments, two of R, R′, R^{L}, R^{L1}, R^{L2}, etc. on different atoms are taken together to form a ring as described herein. For examples, in some embodiments, —X—R^{L} is

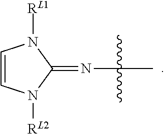

In some embodiments, —X—R^{L} is

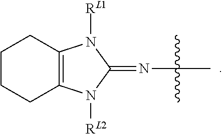

In some embodiments, —X—R^{L} is

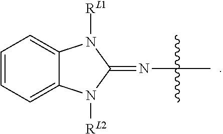

In some embodiments, —X—R^{L} is

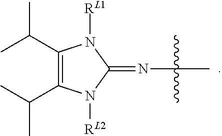

In some embodiments, —X—R^{L} is

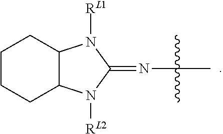

In some embodiments, —X—R^{L} is

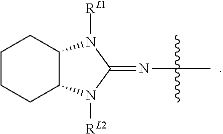

In some embodiments, —X—R^{L} is

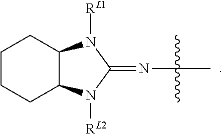

In some embodiments, —X—R^{L} is

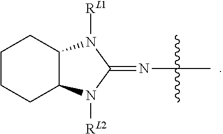

In some embodiments, —X—R^{L} is

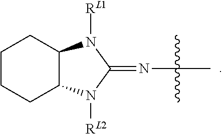

In some embodiments, —X—R^{L} is

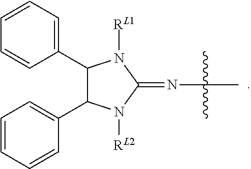

In some embodiments, —N(R′)₂, —N(R)₂, —N(R^{L})₂, —NR′R^{L}, —NR′R^{L1}, —NR′R^{L2}, —NR^{L1}R^{L2}, etc. is a formed ring. In some embodiments, a ring is optionally substituted

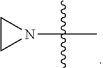 [SMILES: *N1CC1]

In some embodiments, a ring is optionally substituted

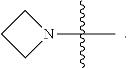 [SMILES: *N1CCC1]

In some embodiments, a ring is optionally substituted

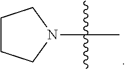 [SMILES: *N1CCCC1]

In some embodiments, a ring is optionally substituted

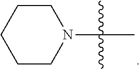 [SMILES: *N1CCCCC1]

In some embodiments, a ring is optionally substituted

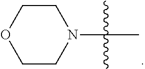 [SMILES: *N1CCOCC1]

In some embodiments, a ring is optionally substituted

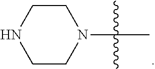 [SMILES: *N1CCNCC1]

In some embodiments, a ring is optionally substituted

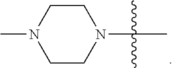 [SMILES: *N1CCN(C)CC1]

In some embodiments, a ring is optionally substituted

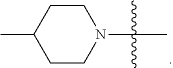 [SMILES: *N1CCC(C)CC1]

In some embodiments, a ring is optionally substituted

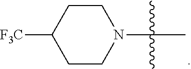 [SMILES: *N1CCC(C(F)(F)F)CC1]

In some embodiments, a ring is optionally substituted

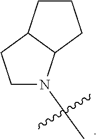 [SMILES: *N1CCC2CCCC21]

In some embodiments, a ring is optionally substituted

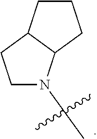 [SMILES: *N1CCC2CCCC21]

In some embodiments, a ring is optionally substituted

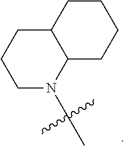 [SMILES: *N1CCCC2CCCCC21]

In some embodiments, a ring is optionally substituted

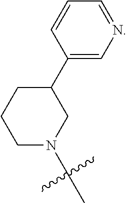 [SMILES: *N1CCCC(c2cccnc2)C1]

In some embodiments, a ring is optionally substituted

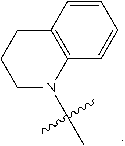 [SMILES: *N1CCCc2ccccc21]

In some embodiments, a ring is optionally substituted

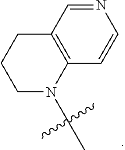 [SMILES: *N1CCCc2cnccc21]

In some embodiments, R^{L1} and R^{L2} are the same. In some embodiments, R^{L1} and R^{L2} are different. In some embodiments, each of R^{L1} and R^{L2} is independently R^{L} as described herein, e.g., below.

In some embodiments, R^{L} is optionally substituted C₁₋₃₀ aliphatic. In some embodiments, R^{L} is optionally substituted C₁₋₃₀ alkyl. In some embodiments, R^{L} is linear, In some embodiments, R^{L} is optionally substituted linear C₁₋₃₀ alkyl. In some embodiments, R^{L} is optionally substituted C₁₋₆ alkyl. In some embodiments, R^{L} is methyl. In some embodiments, R^{L} is ethyl. In some embodiments, R^{L} is n-propyl. In some embodiments, R^{L} is isopropyl. In some embodiments, R^{L} is n-butyl. In some embodiments, R^{L} is tert-butyl. In some embodiments, R^{L} is (E)-CH₂—CH═CH—CH₂—CH₃. In some embodiments, R^{L} is (Z)—CH₂—CH═CH—CH₂—CH₃. In some embodiments, R^{L} is

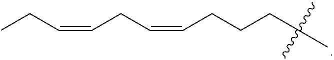 [SMILES: *CCC/C=C\C/C=C\CC]

In some embodiments, R^{L} is

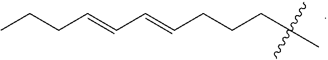 [SMILES: *CCC/C=C/C=C/CCC]

In some embodiments, R^{L} is CH₃(CH₂)₂C≡CC≡C(CH₂)₃—. In some embodiments, R^{L} is CH₃(CH₂)₅C≡C—. In some embodiments, R^{L} optionally substituted aryl. In some embodiments, R^{L} is optionally substituted phenyl. In some embodiments, R^{L} is phenyl substituted with one or more halogen. In some embodiments, R^{L} is phenyl optionally substituted with halogen, —N(R′), or —N(R′)C(O)R′. In some embodiments, R^{L} is phenyl optionally substituted with —Cl, —Br, —F, —N(Me)₂, or —NHCOCH₃. In some embodiments, R^{L} is -L^{L}-R′, wherein L^{L} is an optionally substituted C₁₋₂₀ saturated, partially unsaturated or unsaturated hydrocarbon chain. In some embodiments, such a hydrocarbon chain is linear. In some embodiments, such a hydrocarbon chain is unsubstituted. In some embodiments, L^{L} is (E)-CH₂—CH═CH—. In some embodiments, L^{L} is —CH₂—C≡C—CH₂—. In some embodiments, L^{L} is —(CH₂)₃—. In some embodiments, L^{L} is —(CH₂)₄—. In some embodiments, L^{L} is —(CH₂)ₙ—, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R′ is optionally substituted aryl as described herein. In some embodiments, R′ is optionally substituted phenyl. In some embodiments, R′ is phenyl. In some embodiments, R′ is optionally substituted heteroaryl as described herein. In some embodiments, R′ is 2′-pyridinyl. In some embodiments, R′ is 3′-pyridinyl. In some embodiments, R^{L} is

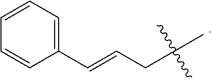 [SMILES: *C/C=C/c1ccccc1]

In some embodiments, R^{L} is

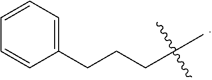 [SMILES: *CCCc1ccccc1]

In some embodiments, R^{L} is

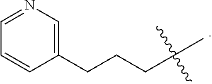 [SMILES: *CCCc1cccnc1]

In some embodiments, R^{L} is -L^{L}-N(R′)₂, wherein each variable is independently as described herein. In some embodiments, each R′ is independently C₁₋₆ aliphatic as described herein. In some embodiments, —N(R′)₂ is —N(CH₃)₂. In some embodiments, —N(R′)₂ is —NH₂. In some embodiments, R^{L} is —(CH₂)ₙ—N(R′)₂, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R^{L} is —(CH₂CH₂O)ₙ—CH₂CH₂—N(R′)₂, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R^{L} is

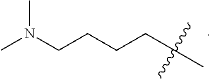 [SMILES: *CCCCN(C)C]

In some embodiments, R^{L} is

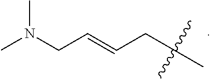 [SMILES: *C/C=C/CN(C)C]

In some embodiments, R^{L} is

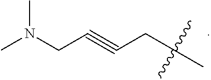 [SMILES: *CC#CCN(C)C]

In some embodiments, R^{L} is —(CH₂)ₙ—NH₂. In some embodiments, R^{L} is —(CH₂CH₂O)ₙ—CH₂CH₂—NH₂. In some embodiments, R^{L} is —(CH₂CH₂O)ₙ—CH₂CH₂—R′, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R^{L} is —(CH₂CH₂O)ₙ—CH₂CH₂CH₃, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R^{L} is —(CH₂CH₂O)ₙ—CH₂CH₂OH, wherein n is 1-30 (e.g., 1-20, 5-30, 6-30, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, etc.). In some embodiments, R^{L} is or comprises a carbohydrate moiety, e.g., GalNAc. In some embodiments, R^{L} is -L^{L}-GalNAc. In some embodiments, R^{L} is

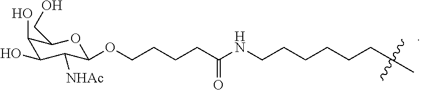 [SMILES: *CCCCCCNC(=O)CCCCOC1OC(CO)C(O)C(O)C1NC(C)=O]

In some embodiments, one or more methylene units of L^{L} are independently replaced with -Cy- (e.g., optionally substituted 1,4-phenylene, a 3-30 membered bivalent optionally substituted monocyclic, bicyclic, or polycyclic cycloaliphatic ring, etc.), —O—, —N(R′)— (e.g., —NH), —C(O)—, —C(O)N(R′)— (e.g., —C(O)NH—), —C(NR′)— (e.g., —C(NH)—), —N(R′)C(O)(N(R′)— (e.g., —NHC(O)NH—), —N(R′)C(NR′)(N(R′)— (e.g., —NHC(NH)NH—), —(CH₂CH₂O)ₙ—, etc. For example, in some embodiments, R^{L} is

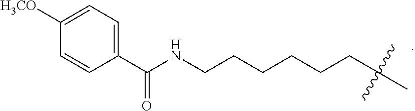 [SMILES: *CCCCCCNC(=O)c1ccc(OC)cc1]

In some embodiments, R^{L} is

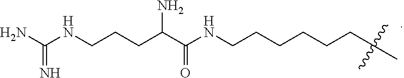 [SMILES: *CCCCCCNC(=O)C(N)CCCNC(=N)N]

In some embodiments, R^{L} is

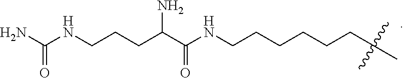 [SMILES: *CCCCCCNC(=O)C(N)CCCNC(N)=O]

In some embodiments, R^{L} is

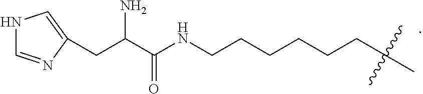 [SMILES: *CCCCCCNC(=O)C(N)Cc1c[nH]cn1]

In some embodiments, R^{L} is

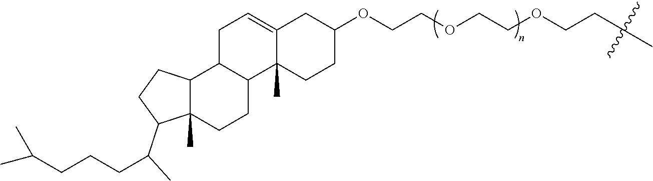

wherein n is 0-20. In some embodiments, R^{L} is or comprises one or more additional chemical moieties (e.g., carbohydrate moieties, GalNAc moieties, etc.) optionally substituted connected through a linker (which can be bivalent or polyvalent). For example, in some embodiments, R^{L} is

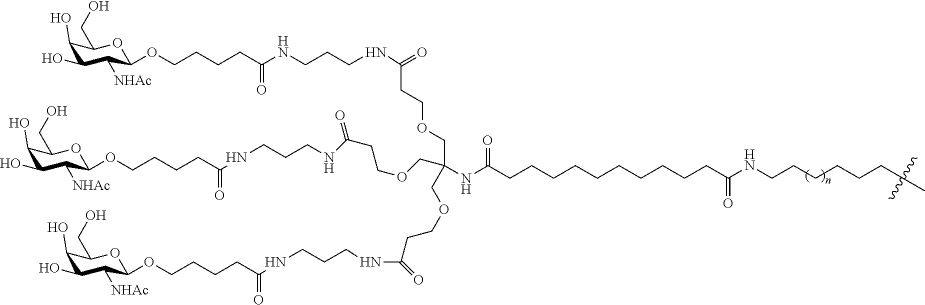

wherein n is 0-20. In some embodiments, R^{L} is

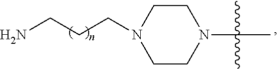

wherein n is 0-20. In some embodiments, R^{L} is R′ as described herein. As described herein, many variable can independently be R′. In some embodiments, R′ is R as described herein. As described herein, various variables can independently be R In some embodiments, R is optionally substituted C₁₋₆ aliphatic. In some embodiments, R is optionally substituted C₁₋₆ alkyl. In some embodiments, R is methyl. In some embodiments, R is optionally substituted cycloaliphatic. In some embodiments, R is optionally substituted cycloalkyl. In some embodiments, R is optionally substituted aryl. In some embodiments, R is optionally substituted phenyl. In some embodiments, R is optionally substituted heteroaryl. In some embodiments, R is optionally substituted heterocyclyl. In some embodiments, R is optionally substituted C₁₋₂₀ heterocyclyl having 1-5 heteroatoms, e.g., one of which is nitrogen. In some embodiments, R is optionally substituted

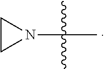 [SMILES: *N1CC1]

In some embodiments, R is optionally substituted

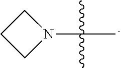 [SMILES: *N1CCC1]

In some embodiments, R is optionally substituted

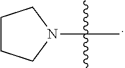 [SMILES: *N1CCCC1]

In some embodiments, R is optionally substituted

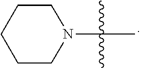 [SMILES: *N1CCCCC1]

In some embodiments, R is optionally substituted

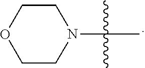 [SMILES: *N1CCOCC1]

In some embodiments, R is optionally substituted

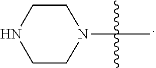 [SMILES: *N1CCNCC1]

In some embodiments, R is optionally substituted

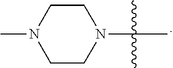 [SMILES: *N1CCN(C)CC1]

In some embodiments, R is optionally substituted

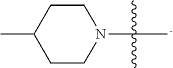 [SMILES: *N1CCC(C)CC1]

In some embodiments, R is optionally substituted

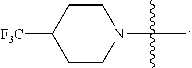 [SMILES: *N1CCC(C(F)(F)F)CC1]

In some embodiments, R is optionally substituted

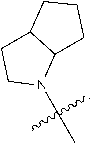 [SMILES: *N1CCC2CCCC21]

In some embodiments, R is optionally substituted

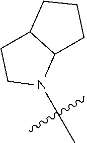 [SMILES: *N1CCC2CCCC21]

In some embodiments, R is optionally substituted

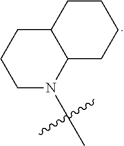 [SMILES: *N1CCCC2CCCCC21]

In some embodiments, R is optionally substituted

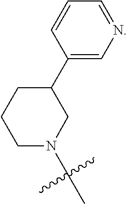 [SMILES: *N1CCCC(c2cccnc2)C1]

In some embodiments, R is optionally substituted

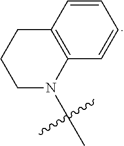 [SMILES: *N1CCCc2ccccc21]

In some embodiments, R is optionally substituted

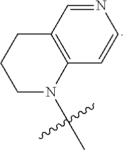 [SMILES: *N1CCCc2cnccc21]

In some embodiments, —X—R^{L} is

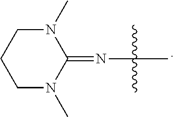 [SMILES: *N=C1N(C)CCCN1C]

In some embodiments, —X—R^{L} is

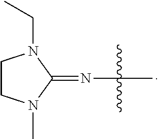 [SMILES: *N=C1N(C)CCN1CC]

In some embodiments, —X—R^{L} is

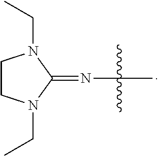 [SMILES: *N=C1N(CC)CCN1CC]

In some embodiments, —X—R^{L} is

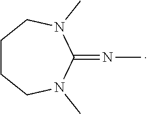

In some embodiments, —X—R^{L} is

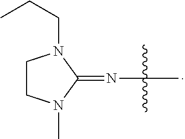 [SMILES: *N=C1N(C)CCN1CCC]

In some embodiments, —X—R^{L} is

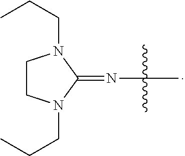 [SMILES: *N=C1N(CCC)CCN1CCC]

In some embodiments, —X—R^{L} is

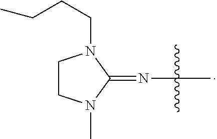 [SMILES: *N=C1N(C)CCN1CCCC]

In some embodiments, —X—R^{L} is

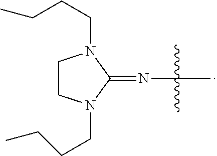 [SMILES: *N=C1N(CCCC)CCN1CCCC]

In some embodiments, —X—R^{L} is

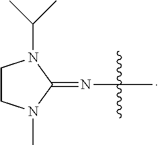 [SMILES: *N=C1N(C)CCN1C(C)C]

In some embodiments, —X—R^{L} is

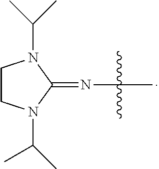 [SMILES: *N=C1N(C(C)C)CCN1C(C)C]

In some embodiments, —X—R^{L} is

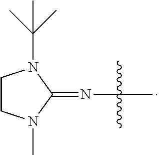 [SMILES: *N=C1N(C)CCN1C(C)(C)C]

In some embodiments, —X—R^{L} is

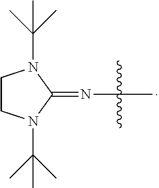 [SMILES: *N=C1N(C(C)(C)C)CCN1C(C)(C)C]

In some embodiments, —X—R^{L} is

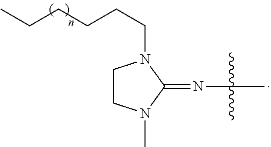

wherein n is 1-20. In some embodiments, —X—R^{L} is

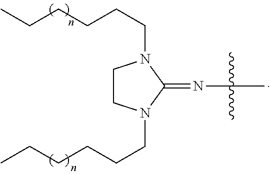

wherein n is 1-20. In some embodiments, —X—R^{L} is selected from:

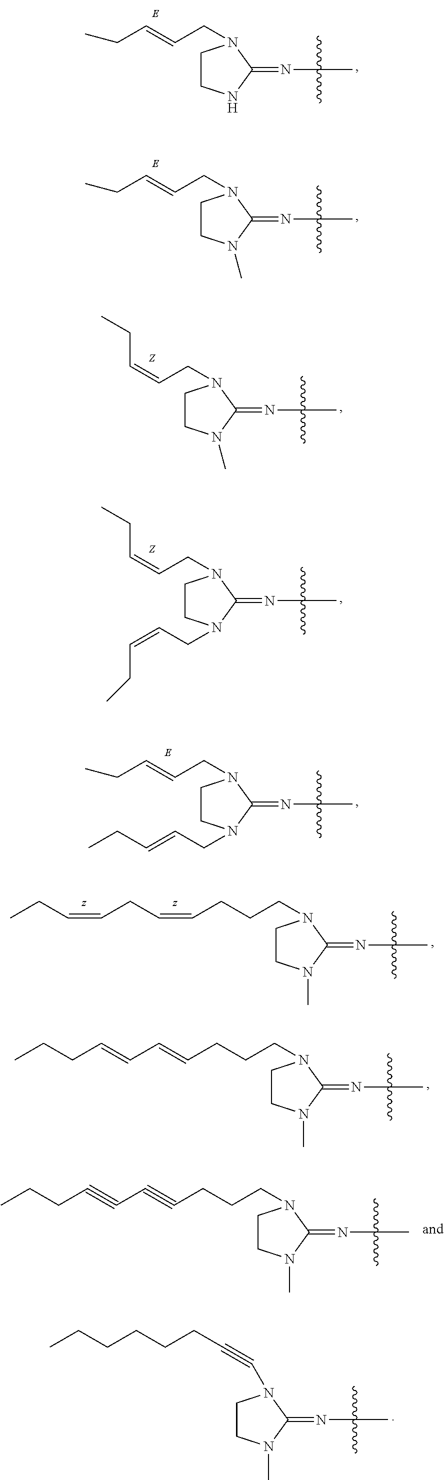

In some embodiments, —X—R^{L} is

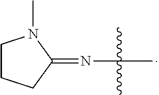 [SMILES: *N=C1CCCN1C]

In some embodiments, —X—R^{L} is

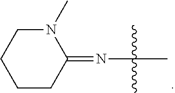 [SMILES: *N=C1CCCCN1C]

In some embodiments, —X—R^{L} is

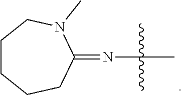 [SMILES: *N=C1CCCCCN1C]

In some embodiments, R^{L} is R″ as described herein. In some embodiments, R^{L} is R as described herein.

In some embodiments, R″ or R^{L} is or comprises an additional chemical moiety. In some embodiments, R″ or R^{L} is or comprises an additional chemical moiety, wherein the additional chemical moiety is or comprises a carbohydrate moiety. In some embodiments, R″ or R^{L} is or comprises a GalNAc. In some embodiments, R^{L} or R″ is replaced with, or is utilized to connect to, an additional chemical moiety.

In some embodiments, X is —O—. In some embodiments, X is —S—. In some embodiments, X is -L^{L}-N(-L^{L}-R^{L})-L^{L}-. In some embodiments, X is —N(-L^{L}-R^{L})-L^{L} In some embodiments, X is -L^{L}-N(-L^{L}-R^{L})—. In some embodiments, X is —N(-L^{L}-R^{L})—. In some embodiments, X is -L^{L}-N═C(-L^{L}-R^{L})-L^{L}-. In some embodiments, X is —N═C(-L^{L}-R^{L})-L^{L}-. In some embodiments, X is -L^{L}-N═C(-L^{L}-R^{L})—. In some embodiments, X is —N═C(-L^{L}-R^{L})—. In some embodiments, X is L^{L}. In some embodiments, X is a covalent bond. In some embodiments, Y is a covalent bond. In some embodiments, Y is —O—. In some embodiments, Y is —N(R′)—. In some embodiments, Z is a covalent bond. In some embodiments, Z is —O—. In some embodiments, Z is —N(R′)—. In some embodiments, R′ is R In some embodiments, R is —H. In some embodiments, R is optionally substituted C₁₋₆ aliphatic. In some embodiments, R is methyl. In some embodiments, R is ethyl. In some embodiments, R is propyl. In some embodiments, R is optionally substituted phenyl. In some embodiments, R is phenyl.

As described herein, various variables in structures in the present disclosure can be or comprise R Suitable embodiments for R are described extensively in the present disclosure. As appreciated by those skilled in the art, R embodiments described for a variable that can be R may also be applicable to another variable that can be R Similarly, embodiments described for a component/moiety (e.g., L) for a variable may also be applicable to other variables that can be or comprise the component/moiety.

In some embodiments, R″ is R′. In some embodiments, R″ is —N(R′)₂.

In some embodiments, —X—R^{L} is —SH. In some embodiments, —X—R^{L} is —OH. In some embodiments, —X—R^{L} is —N(R′)₂. In some embodiments, each R′ is independently optionally substituted C₁₋₆ aliphatic. In some embodiments, each R′ is independently methyl.

In some embodiments, a non-negatively charged internucleotidic linkage has the structure of —OP(═O)(—N═C((N(R′)₂)—O—. In some embodiments, a R′ group of one N(R′)₂ is R, a R′ group of the other N(R′)₂ is R, and the two R groups are taken together with their intervening atoms to form an optionally substituted ring, e.g., a 5-membered ring as in n001. In some embodiments, each R′ is independently R, wherein each R is independently optionally substituted C₁₋₆ aliphatic.

In some embodiments, —X—R^{L} is —N═C(-L^{L}-R′)₂. In some embodiments, —X—R^{L} is —N═C(-L^{L1}-L^{L2}-L^{L3}-R′)₂, wherein each L^{L1}, L^{L2} and L^{L3} is independently L″, wherein each L″ is independently a covalent bond, or a bivalent, optionally substituted, linear or branched group selected from a C₁₋₁₀ aliphatic group and a C₁₋₁₀ heteroaliphatic group having 1-5 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L}. In some embodiments, L^{L2} is -Cy-. In some embodiments, L^{L1} is a covalent bond. In some embodiments, L^{L3} is a covalent bond. In some embodiments, —X—R^{L} is —N═C(-L^{L1}-Cy-L^{L3}-R′)₂. In some embodiments, —X—R^{L} is [symbol] . In some embodiments, —X—R^{L} is [symbol] . In some embodiments, —X—R^{L} is [symbol] . In some embodiments, —X—R^{L} is [symbol] . In some embodiments, —X—R^{L} is [symbol] . In some embodiments, —X—R^{L} is [symbol] .

In some embodiments, as utilized in the present disclosure, L is covalent bond. In some embodiments, L is a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L}. In some embodiments, L is a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced by an optionally substituted group selected from —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L}. In some embodiments, L is a bivalent, optionally substituted, linear or branched group selected from a C₁₋₁₀ aliphatic group and a C₁₋₁₀ heteroaliphatic group having 1-10 heteroatoms, wherein one or more methylene units are optionally and independently replaced by an optionally substituted group selected from —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more nitrogen or carbon atoms are optionally and independently replaced with Cy^{L}. In some embodiments, one or more methylene units are optionally and independently replaced by an optionally substituted group selected from —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—.

In some embodiments, an internucleotidic linkage is a phosphoryl guanidine internucleotidic linkage. In some embodiments, —X—R^{L} is —N═C[N(R′)₂]₂. In some embodiments, each R′ is independently R. In some embodiments, R is optionally substituted C₁₋₆ aliphatic. In some embodiments, R is methyl. In some embodiments, —X—R^{L} is

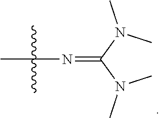 [SMILES: *N=C(N(C)C)N(C)C]

In some embodiments, one R′ on a nitrogen atom is taken with a R′ on the other nitrogen to form a ring as described herein.

In some embodiments, —X—R^{L} is

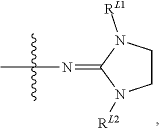

wherein R¹ and R² are independently R′. In some embodiments, —X—R^{L} is

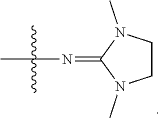 [SMILES: *N=C1N(C)CCN1C]

In some embodiments, —X—R^{L} is

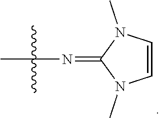 [SMILES: *N=c1n(C)ccn1C]

In some embodiments, two R′ on the same nitrogen are taken together to form a ring as described herein. In some embodiments, —X—R^{L} is

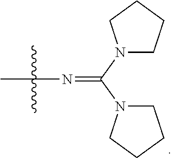 [SMILES: *N=C(N1CCCC1)N1CCCC1]

In some embodiments, —X—R^{L} is

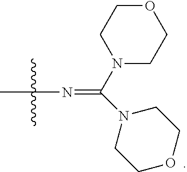 [SMILES: *N=C(N1CCOCC1)N1CCOCC1]

In some embodiments, —X—R^{L} is

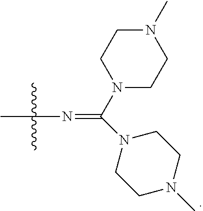 [SMILES: *N=C(N1CCN(C)CC1)N1CCN(C)CC1]

In some embodiments, —X—R^{L} is

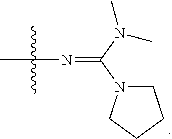 [SMILES: *N=C(N(C)C)N1CCCC1]

In some embodiments, —X—R^{L} is

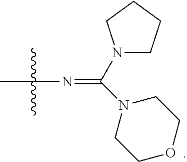 [SMILES: *N=C(N1CCCC1)N1CCOCC1]

In some embodiments, —X—R^{L} is

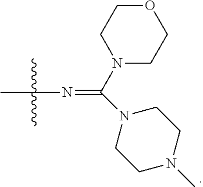 [SMILES: *N=C(N1CCOCC1)N1CCN(C)CC1]

In some embodiments, —X—R^{L} is

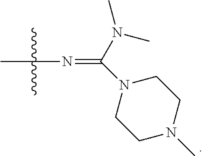 [SMILES: *N=C(N(C)C)N1CCN(C)CC1]

In some embodiments, —X—R^{L} is

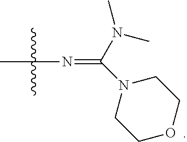 [SMILES: *N=C(N(C)C)N1CCOCC1]

In some embodiments, —X—R^{L}

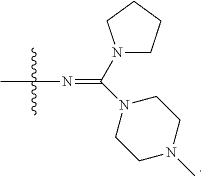 [SMILES: *N=C(N1CCCC1)N1CCN(C)CC1]

In some embodiments, —X—R^{L} is R as described herein. In some embodiments, R is not hydrogen. In some embodiments, R is optionally substituted C₁₋₆ aliphatic. In some embodiments, R is optionally substituted C₁₋₆ alkyl. In some embodiments, R is methyl.

In some embodiments, —X—R^{L} is selected from Tables below. In some embodiments, X is as described herein. In some embodiments, R^{L} is as described herein. In some embodiments, a linkage has the structure of —Y—P^{L}(—X—R^{L})—Z—, wherein —X—R^{L} is selected from Tables below, and each other variable is independently as described herein. In some embodiments, a linkage has the structure of or comprises —P(OX—X—R^{L})—, wherein —X—R^{L} is selected from Tables below. In some embodiments, a linkage has the structure of or comprises —P(S)—X—R^{L})—, wherein —X—R^{L} is selected from Tables below. In some embodiments, a linkage has the structure of or comprises —P(—X—R^{L})—, wherein —X—R^{L} is selected from Tables below. In some embodiments, a linkage has the structure of or comprises —O—P(O)(—X—R^{L})—O—, wherein —X—R^{L} is selected from Tables below. In some embodiments, a linkage has the structure of or comprises —O—P(S)(—X—R^{L})—, wherein —X—R^{L} is selected from Tables below. In some embodiments, a linkage has the structure of or comprises —O—P(—X—R^{L})—O—, wherein —X—R^{L} is selected from Tables below. In some embodiments, a linkage has the structure of —O—P(O)(—X—R^{L})—, wherein —X—R^{L} is selected from Tables below. In some embodiments, a linkage has the structure of —O—P(S)(—X—R^{L})—O—, wherein —X—R^{L} is selected from Tables below. In some embodiments, a linkage has the structure of —O—P(—X—R^{L})—O—, wherein —X—R^{L} is selected from Tables below. In some embodiments, the Tables below, n is 0-20 or as described herein.

TABLE L-1
Certain useful moieties bonded to linkage phosphorus (e.g., —X—R^{L}).

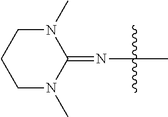

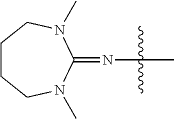

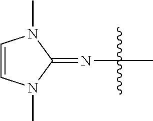

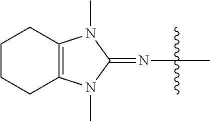

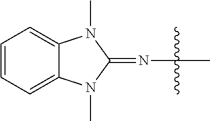

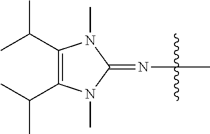

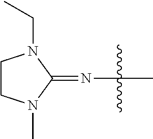

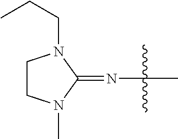

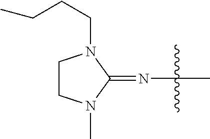

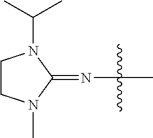

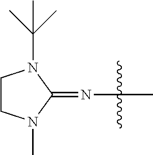

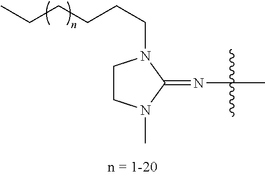

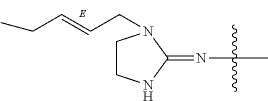

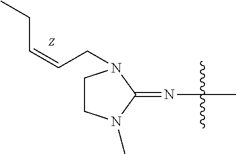

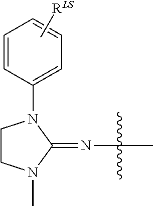

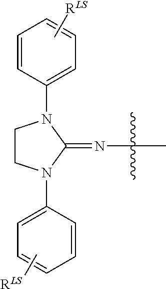

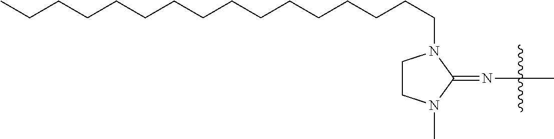

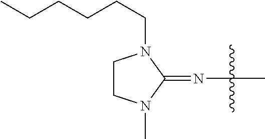

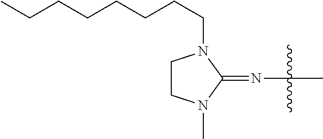

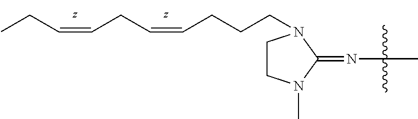

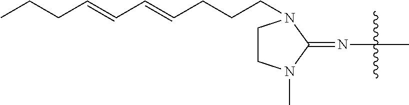

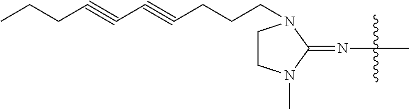

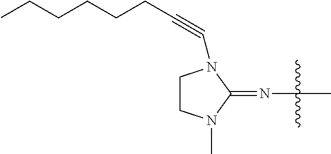

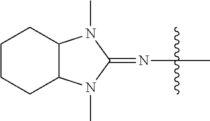

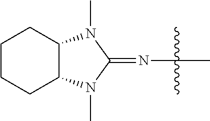

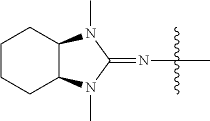

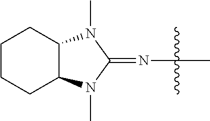

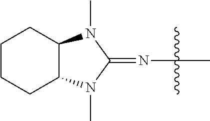

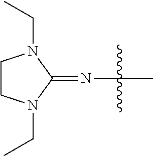

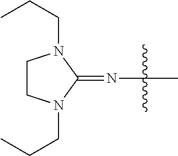

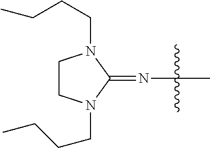

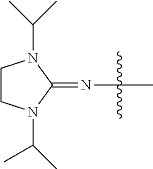

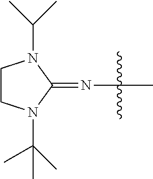

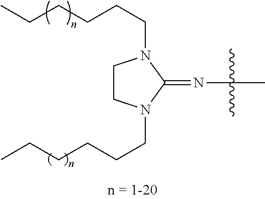

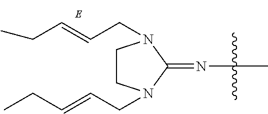

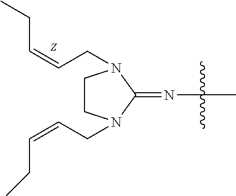

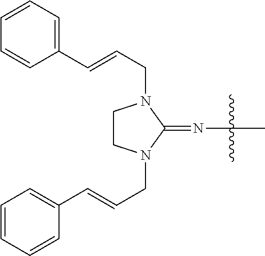

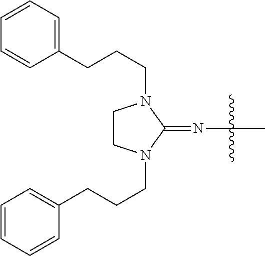

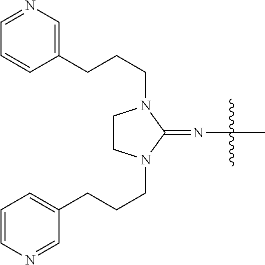

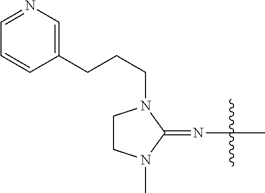

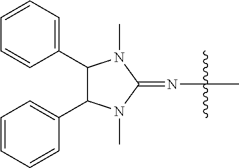

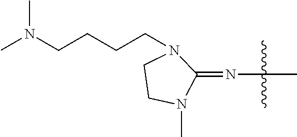

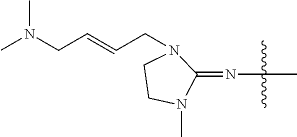

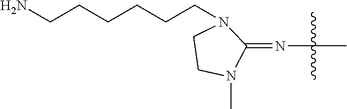

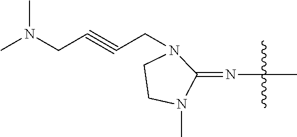

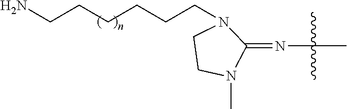

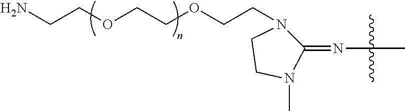

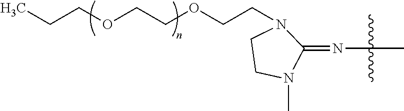

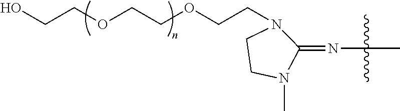

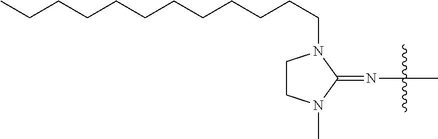

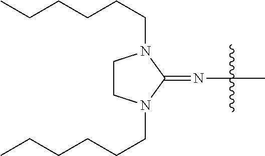

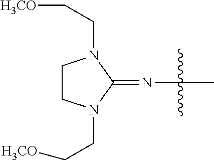

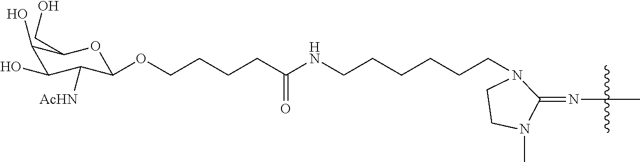

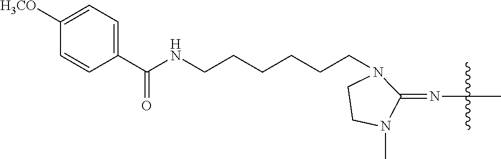

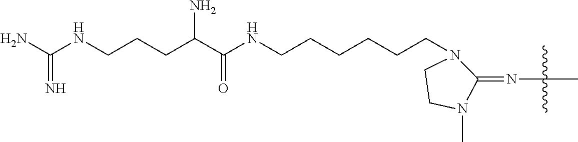

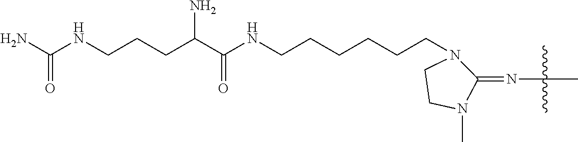

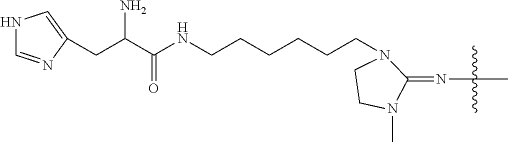

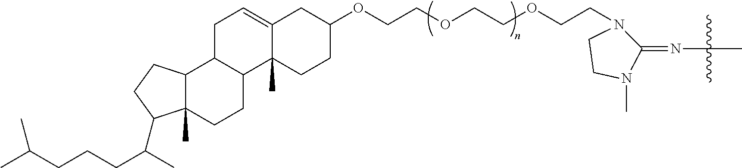

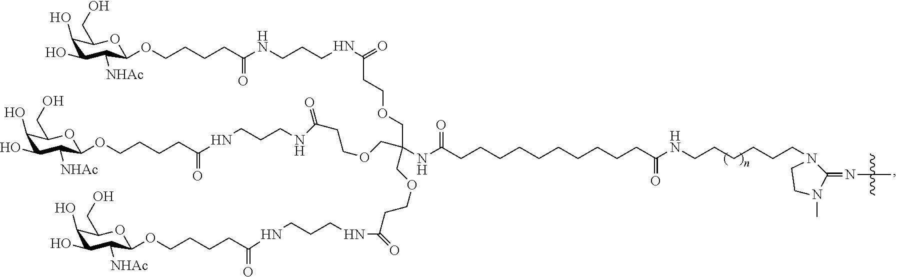

wherein each R^{LS} is independently R^{s}. In some embodiments, each R^{LS} is independently —Cl, —Br, —F, —N(Me)₂, or —NHCOCH₃.

TABLE L-2
Certain useful moieties bonded to linkage phosphorus (e.g., —X—R^{L}).

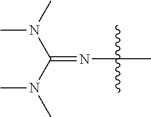 [SMILES: *N=C(N(C)C)N(C)C]

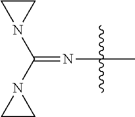 [SMILES: *N=C(N1CC1)N1CC1]

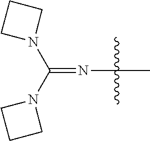 [SMILES: *N=C(N1CCC1)N1CCC1]

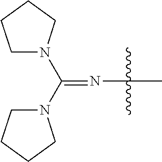 [SMILES: *N=C(N1CCCC1)N1CCCC1]

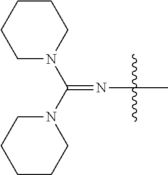 [SMILES: *N=C(N1CCCCC1)N1CCCCC1]

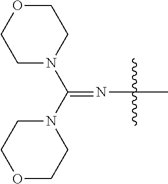 [SMILES: *N=C(N1CCOCC1)N1CCOCC1]

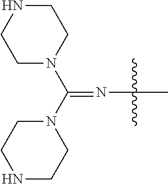 [SMILES: *N=C(N1CCNCC1)N1CCNCC1]

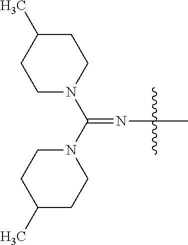 [SMILES: *N=C(N1CCC(C)CC1)N1CCC(C)CC1]

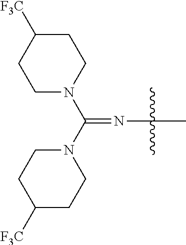 [SMILES: *N=C(N1CCC(C(F)(F)F)CC1)N1CCC(C(F)(F)F)CC1]

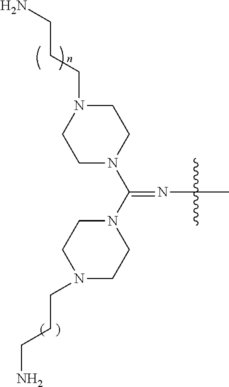

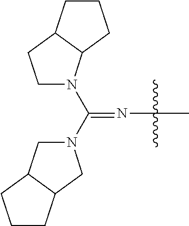 [SMILES: *N=C(N1CC2CCCC2C1)N1CCC2CCCC21]

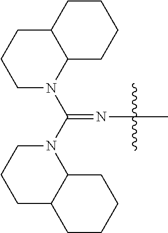 [SMILES: *N=C(N1CCCC2CCCCC21)N1CCCC2CCCCC21]

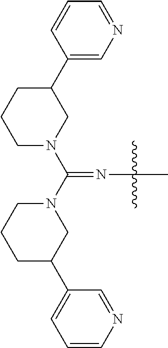 [SMILES: *N=C(N1CCCC(c2cccnc2)C1)N1CCCC(c2cccnc2)C1]

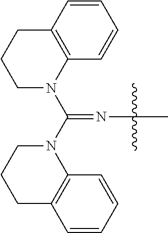 [SMILES: *N=C(N1CCCc2ccccc21)N1CCCc2ccccc21]

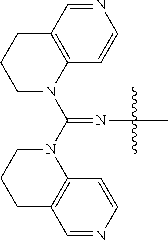 [SMILES: *N=C(N1CCCc2cnccc21)N1CCCc2cnccc21]

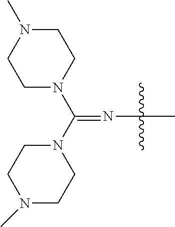 [SMILES: *N=C(N1CCN(C)CC1)N1CCN(C)CC1]

TABLE L-3
Certain useful moieties bonded to linkage phosphorus (e.g., —X—R^{L}).

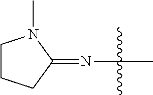 [SMILES: *N=C1CCCN1C]

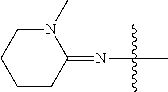 [SMILES: *N=C1CCCCN1C]

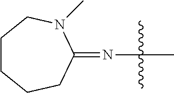 [SMILES: *N=C1CCCCCN1C]

TABLE L-4
Certain useful moieties bonded to linkage phosphorus (e.g., —X—R^{L}).

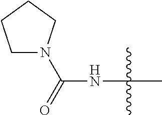 [SMILES: *NC(=O)N1CCCC1]

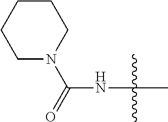 [SMILES: *NC(=O)N1CCCCC1]

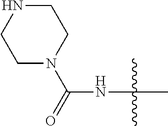 [SMILES: *NC(=O)N1CCNCC1]

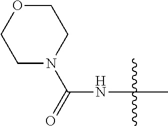 [SMILES: *NC(=O)N1CCOCC1]

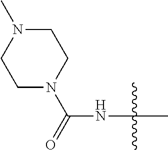 [SMILES: *NC(=O)N1CCN(C)CC1]

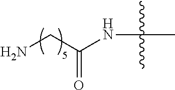

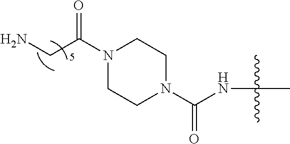

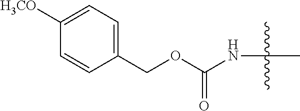 [SMILES: *NC(=O)OCc1ccc(OC)cc1]

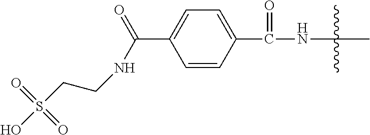 [SMILES: *NC(=O)c1ccc(C(=O)NCCS(=O)(=O)O)cc1]

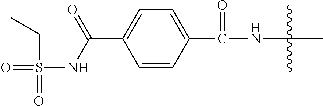 [SMILES: *NC(=O)c1ccc(C(=O)NS(=O)(=O)CC)cc1]

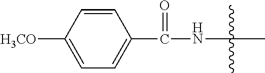 [SMILES: *NC(=O)c1ccc(OC)cc1]

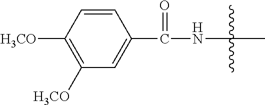 [SMILES: *NC(=O)c1ccc(OC)c(OC)c1]

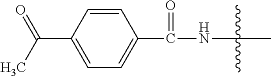 [SMILES: *NC(=O)c1ccc(C(C)=O)cc1]

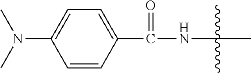 [SMILES: *NC(=O)c1ccc(N(C)C)cc1]

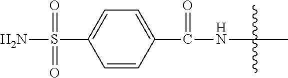 [SMILES: *NC(=O)c1ccc(S(N)(=O)=O)cc1]

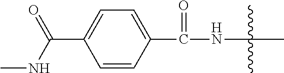 [SMILES: *NC(=O)c1ccc(C(=O)NC)cc1]

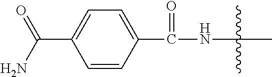 [SMILES: *NC(=O)c1ccc(C(N)=O)cc1]

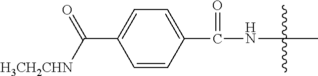

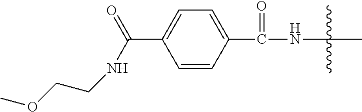 [SMILES: *NC(=O)c1ccc(C(=O)NCCOC)cc1]

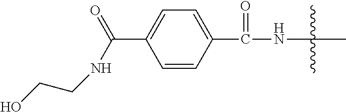 [SMILES: *NC(=O)c1ccc(C(=O)NCCO)cc1]

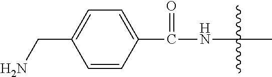 [SMILES: *NC(=O)c1ccc(CN)cc1]

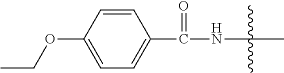 [SMILES: *NC(=O)c1ccc(OCC)cc1]

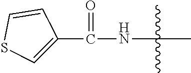 [SMILES: *NC(=O)c1ccsc1]

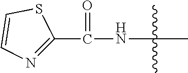 [SMILES: *NC(=O)c1nccs1]

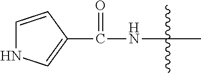 [SMILES: *NC(=O)c1cc[nH]c1]

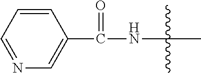 [SMILES: *NC(=O)c1cccnc1]

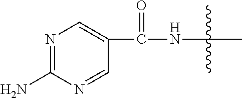 [SMILES: *NC(=O)c1cnc(N)nc1]

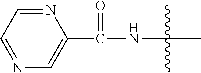 [SMILES: *NC(=O)c1cnccn1]

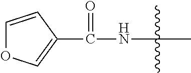 [SMILES: *NC(=O)c1ccoc1]

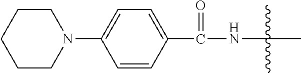 [SMILES: *NC(=O)c1ccc(N2CCCCC2)cc1]

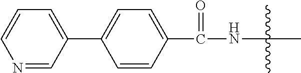 [SMILES: *NC(=O)c1ccc(-c2cccnc2)cc1]

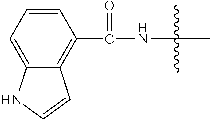 [SMILES: *NC(=O)c1cccc2[nH]ccc12]

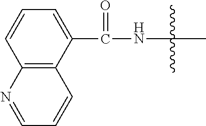 [SMILES: *NC(=O)c1cccc2ncccc12]

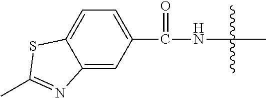 [SMILES: *NC(=O)c1ccc2sc(C)nc2c1]

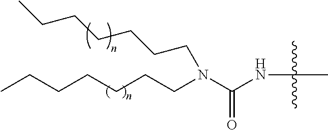

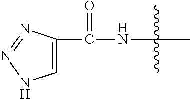 [SMILES: *NC(=O)c1c[nH]nn1]

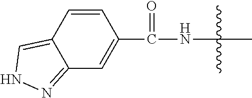 [SMILES: *NC(=O)c1ccc2c[nH]nc2c1]

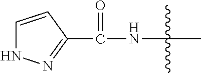 [SMILES: *NC(=O)c1cc[nH]n1]

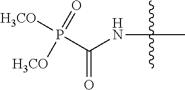 [SMILES: *NC(=O)P(=O)(OC)OC]

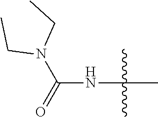 [SMILES: *NC(=O)N(CC)CC]

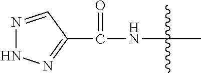 [SMILES: *NC(=O)c1cn[nH]n1]

TABLE L-5
Certain useful moieties bonded to linkage phosphorus (e.g., —X—R^{L}).

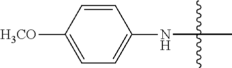 [SMILES: *Nc1ccc(OC)cc1]

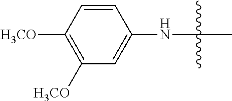 [SMILES: *Nc1ccc(OC)c(OC)c1]

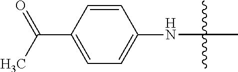 [SMILES: *Nc1ccc(C(C)=O)cc1]

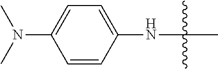 [SMILES: *Nc1ccc(N(C)C)cc1]

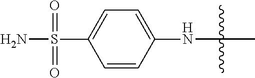 [SMILES: *Nc1ccc(S(N)(=O)=O)cc1]

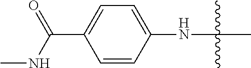 [SMILES: *Nc1ccc(C(=O)NC)cc1]

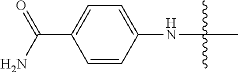 [SMILES: *Nc1ccc(C(N)=O)cc1]

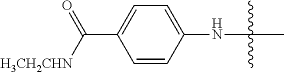

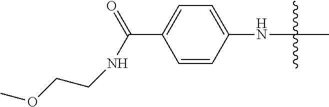 [SMILES: *Nc1ccc(C(=O)NCCOC)cc1]

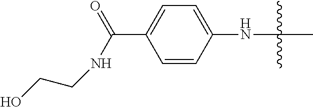 [SMILES: *Nc1ccc(C(=O)NCCO)cc1]

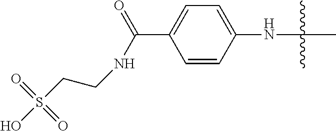 [SMILES: *Nc1ccc(C(=O)NCCS(=O)(=O)O)cc1]

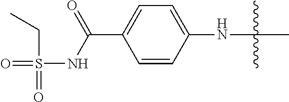 [SMILES: *Nc1ccc(C(=O)NS(=O)(=O)CC)cc1]

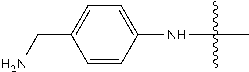 [SMILES: *Nc1ccc(CN)cc1]

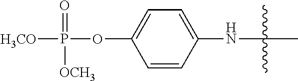 [SMILES: *Nc1ccc(OP(=O)(OC)OC)cc1]

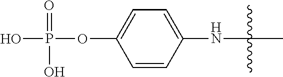 [SMILES: *Nc1ccc(OP(=O)(O)O)cc1]

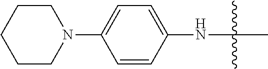 [SMILES: *Nc1ccc(N2CCCCC2)cc1]

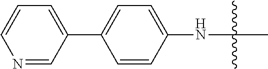 [SMILES: *Nc1ccc(-c2cccnc2)cc1]

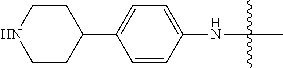 [SMILES: *Nc1ccc(C2CCNCC2)cc1]

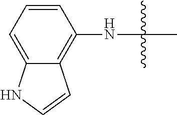 [SMILES: *Nc1cccc2[nH]ccc12]

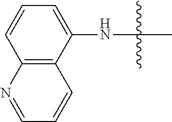 [SMILES: *Nc1cccc2ncccc12]

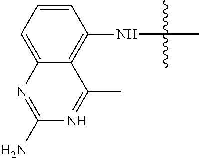 [SMILES: *Nc1cccc2nc(N)nc(C)c12]

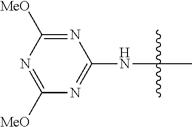 [SMILES: *Nc1nc(OC)nc(OC)n1]

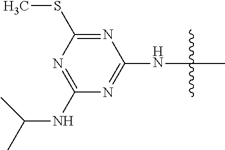 [SMILES: *Nc1nc(NC(C)C)nc(SC)n1]

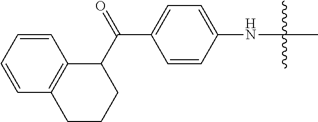 [SMILES: *Nc1ccc(C(=O)C2CCCc3ccccc32)cc1]

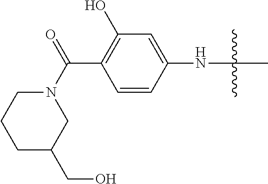 [SMILES: *Nc1ccc(C(=O)N2CCCC(CO)C2)c(O)c1]

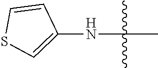 [SMILES: *Nc1ccsc1]

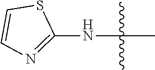 [SMILES: *Nc1nccs1]

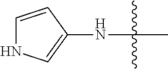 [SMILES: *Nc1cc[nH]c1]

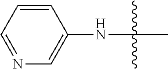 [SMILES: *Nc1cccnc1]

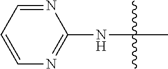 [SMILES: *Nc1ncccn1]

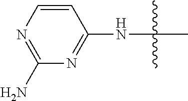 [SMILES: *Nc1ccnc(N)n1]

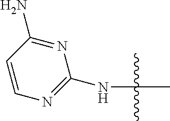 [SMILES: *Nc1nccc(N)n1]

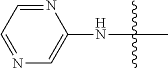 [SMILES: *Nc1cnccn1]

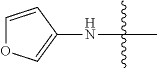 [SMILES: *Nc1ccoc1]

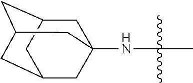 [SMILES: *NC12CC3CC(CC(C3)C1)C2]

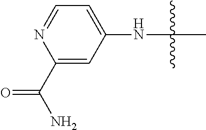 [SMILES: *Nc1ccnc(C(N)=O)c1]

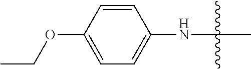 [SMILES: *Nc1ccc(OCC)cc1]

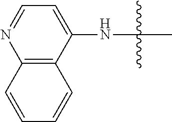 [SMILES: *Nc1ccnc2ccccc12]

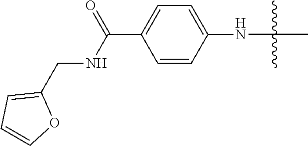 [SMILES: *Nc1ccc(C(=O)NCc2ccco2)cc1]

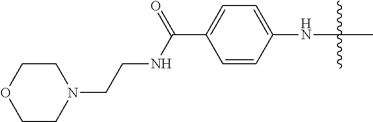 [SMILES: *Nc1ccc(C(=O)NCCN2CCOCC2)cc1]

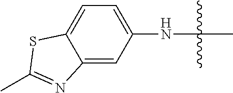 [SMILES: *Nc1ccc2sc(C)nc2c1]

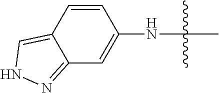 [SMILES: *Nc1ccc2c[nH]nc2c1]

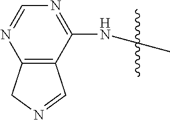 [SMILES: *Nc1ncnc2c1C=NC2]

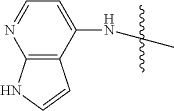 [SMILES: *Nc1ccnc2[nH]ccc12]

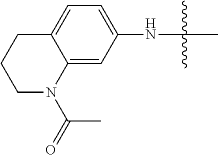 [SMILES: *Nc1ccc2c(c1)N(C(C)=O)CCC2]

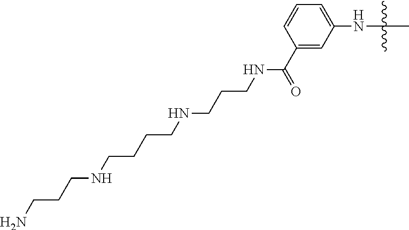 [SMILES: *Nc1cccc(C(=O)NCCCNCCCCNCCCN)c1]

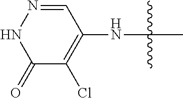 [SMILES: *Nc1cn[nH]c(=O)c1Cl]

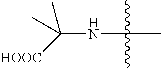 [SMILES: *NC(C)(C)C(=O)O]

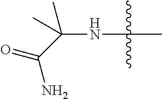 [SMILES: *NC(C)(C)C(N)=O]

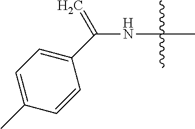 [SMILES: *NC(=C)c1ccc(C)cc1]

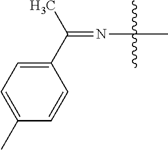 [SMILES: *N=C(C)c1ccc(C)cc1]

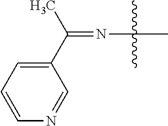 [SMILES: *N=C(C)c1cccnc1]

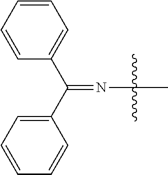 [SMILES: *N=C(c1ccccc1)c1ccccc1]

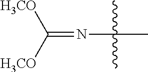 [SMILES: *N=C(OC)OC]

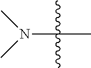 [SMILES: *N(C)C]

TABLE L-6
Certain useful moieties bonded to linkage phosphorus (e.g., —X—R^{L}).

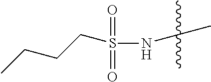 [SMILES: *NS(=O)(=O)CCCC]

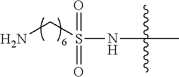

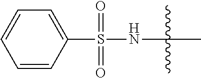 [SMILES: *NS(=O)(=O)c1ccccc1]

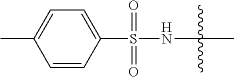 [SMILES: *NS(=O)(=O)c1ccc(C)cc1]

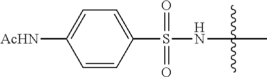 [SMILES: *NS(=O)(=O)c1ccc(NC(C)=O)cc1]

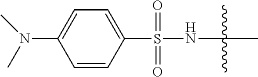 [SMILES: *NS(=O)(=O)c1ccc(N(C)C)cc1]

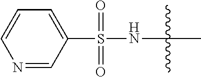 [SMILES: *NS(=O)(=O)c1cccnc1]

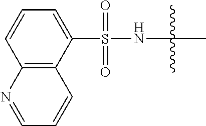 [SMILES: *NS(=O)(=O)c1cccc2ncccc12]

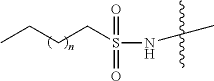

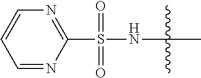 [SMILES: *NS(=O)(=O)c1ncccn1]

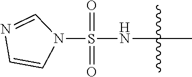 [SMILES: *NS(=O)(=O)n1ccnc1]

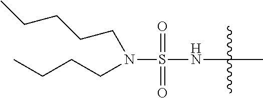 [SMILES: *NS(=O)(=O)N(CCCC)CCCCC]

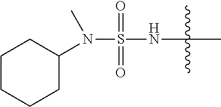 [SMILES: *NS(=O)(=O)N(C)C1CCCCC1]

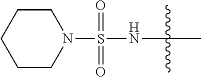 [SMILES: *NS(=O)(=O)N1CCCCC1]

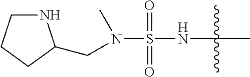 [SMILES: *NS(=O)(=O)N(C)CC1CCCN1]

In some embodiments, an internucleotidic linkage, e.g., a non-negatively charged internucleotidic linkage or a neutral internucleotidic linkage, has the structure of -L^{L1}-Cy^{IL}-L^{L2}-. In some embodiments, L^{L1} is bonded to a 3′-carbon of a sugar. In some embodiments, L^{L2} is bonded to a 5′-carbon of a sugar. In some embodiments, L^{L1} is —O—CH₂—. In some embodiments, L^{L2} is a covalent bond. In some embodiments, L^{L2} is a —N(R′)—. In some embodiments, L^{L2} is a —NH—. In some embodiments, L^{L2} is bonded to a 5′-carbon of a sugar, which 5′-carbon is substituted with ═O. In some embodiments, Cy^{IL} is optionally substituted 3-10 membered saturated, partially unsaturated, or aromatic ring having 0-5 heteroatoms. In some embodiments, Cy^{IL} is an optionally substituted triazole ring. In some embodiments, Cy^{IL} is

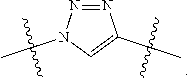 [SMILES: *c1cn(*)nn1]

In some embodiments, a linkage is

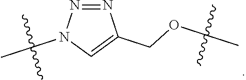 [SMILES: *OCc1cn(*)nn1]

In some embodiments, a non-negatively charged internucleotidic linkage has the structure of —OP(═W)(—N(R′)₂)—O—.

In some embodiments, R′ is R. In some embodiments, R′ is H. In some embodiments, R′ is —C(O)R. In some embodiments, R′ is —C(O)OR. In some embodiments, R′ is —S(O)₂R

In some embodiments, R″ is —NHR′. In some embodiments, —N(R′)₂ is —NHR′.

As described herein, some embodiments, R is H. In some embodiments, R is optionally substituted C₁₋₆ aliphatic. In some embodiments, R is optionally substituted C₁₋₆ alkyl. In some embodiments, R is methyl. In some embodiments, R is substituted methyl. In some embodiments, R is ethyl. In some embodiments, R is substituted ethyl.

In some embodiments, as described herein, a non-negatively charged internucleotidic linkage is a neutral internucleotidic linkage.

In some embodiments, a modified internucleotidic linkage (e.g., a non-negatively charged internucleotidic linkage) comprises optionally substituted triazolyl. In some embodiments, R′ is or comprises optionally substituted triazolyl. In some embodiments, a modified internucleotidic linkage (e.g., a non-negatively charged internucleotidic linkage) comprises optionally substituted alkynyl. In some embodiments, R′ is optionally substituted alkynyl. In some embodiments, R′ comprises an optionally substituted triple bond. In some embodiments, a modified internucleotidic linkage comprises a triazole or alkyne moiety. In some embodiments, R′ is or comprises an optionally substituted triazole or alkyne moiety. In some embodiments, a triazole moiety, e.g., a triazolyl group, is optionally substituted. In some embodiments, a triazole moiety, e.g., a triazolyl group) is substituted. In some embodiments, a triazole moiety is unsubstituted. In some embodiments, a modified internucleotidic linkage comprises an optionally substituted guanidine moiety. In some embodiments, a modified internucleotidic linkage comprises an optionally substituted cyclic guanidine moiety. In some embodiments, R′, R^{L}, or —X—R^{L}, is or comprises an optionally substituted guanidine moiety. In some embodiments, R′, R^{L}, or —X—R^{L}, is or comprises an optionally substituted cyclic guanidine moiety. In some embodiments, R′, R^{L}, or —X—R^{L} comprises an optionally substituted cyclic guanidine moiety and an internucleotidic linkage has the structure of:

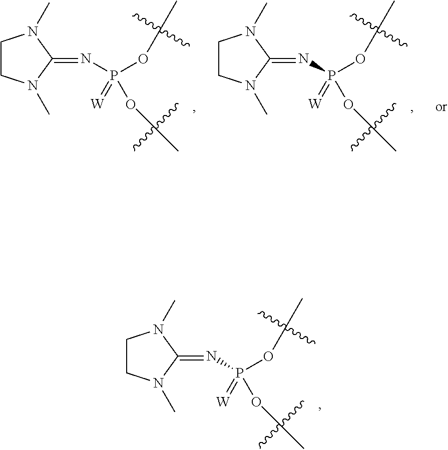

wherein W is O or S. In some embodiments, W is O. In some embodiments, W is S. In some embodiments, anon-negatively charged internucleotidic linkage is stereochemically controlled.

In some embodiments, a non-negatively charged internucleotidic linkage or a neutral internucleotidic linkage is an internucleotidic linkage comprising a triazole moiety. In some embodiments, a non-negatively charged internucleotidic linkage or a non-negatively charged internucleotidic linkage comprises an optionally substituted triazolyl group. In some embodiments, an internucleotidic linkage comprising a triazole moiety (e.g., an optionally substituted triazolyl group) has the structure of

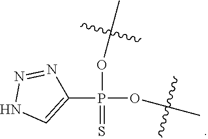 [SMILES: *OP(=S)(O*)c1c[nH]nn1]

In some embodiments, an internucleotidic linkage comprising a triazole moiety has the structure of

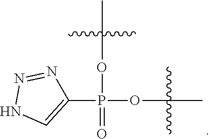 [SMILES: *OP(=O)(O*)c1c[nH]nn1]

In some embodiments, an internucleotidic linkage, e.g., a non-negatively charged internucleotidic linkage, a neutral internucleotidic linkage, comprises a cyclic guanidine moiety. In some embodiments, an internucleotidic linkage comprising a cyclic guanidine moiety has the structure of

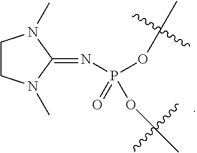 [SMILES: *OP(=O)(N=C1N(C)CCN1C)O*]

In some embodiments, a non-negatively charged internucleotidic linkage, or a neutral internucleotidic linkage, is or comprising a structure selected from

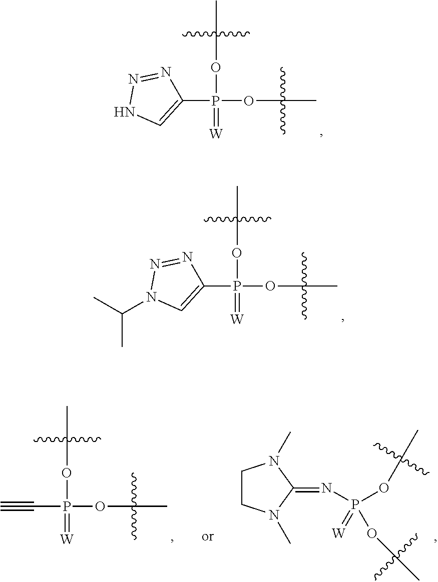

wherein W is O or S.

In some embodiments, an internucleotidic linkage comprises a Tmg group

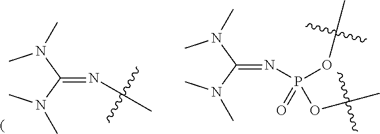

In some embodiments, an internucleotidic linkage comprises a Tmg group and has the structure of

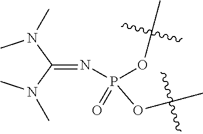 [SMILES: *OP(=O)(N=C(N(C)C)N(C)C)O*]

(the “Tmg internucleotidic linkage”). In some embodiments, neutral internucleotidic linkages include internucleotidic linkages of PNA and PMO, and an Tmg internucleotidic linkage.

In some embodiments, a non-negatively charged internucleotidic linkage comprises an optionally substituted 3-20 membered heterocyclyl or heteroaryl group having 1-10 heteroatoms. In some embodiments, a non-negatively charged internucleotidic linkage comprises an optionally substituted 3-20 membered heterocyclyl or heteroaryl group having 1-heteroatoms, wherein at least one heteroatom is nitrogen. In some embodiments, such a heterocyclyl or heteroaryl group is of a 5-membered ring. In some embodiments, such a heterocyclyl or heteroaryl group is of a 6-membered ring.

In some embodiments, a non-negatively charged internucleotidic linkage comprises an optionally substituted 5-20 membered heteroaryl group having 1-10 heteroatoms. In some embodiments, a non-negatively charged internucleotidic linkage comprises an optionally substituted 5-20 membered heteroaryl group having 1-10 heteroatoms, wherein at least one heteroatom is nitrogen. In some embodiments, a non-negatively charged internucleotidic linkage comprises an optionally substituted 5-6 membered heteroaryl group having 1-4 heteroatoms, wherein at least one heteroatom is nitrogen. In some embodiments, a non-negatively charged internucleotidic linkage comprises an optionally substituted 5-membered heteroaryl group having 1-4 heteroatoms, wherein at least one heteroatom is nitrogen. In some embodiments, a heteroaryl group is directly bonded to a linkage phosphorus. In some embodiments, a non-negatively charged internucleotidic linkage comprises an optionally substituted 5-20 membered heterocyclyl group having 1-10 heteroatoms. In some embodiments, a non-negatively charged internucleotidic linkage comprises an optionally substituted 5-20 membered heterocyclyl group having 1-10 heteroatoms, wherein at least one heteroatom is nitrogen. In some embodiments, a non-negatively charged internucleotidic linkage comprises an optionally substituted 5-6 membered heterocyclyl group having 1-4 heteroatoms, wherein at least one heteroatom is nitrogen. In some embodiments, a non-negatively charged internucleotidic linkage comprises an optionally substituted 5-membered heterocyclyl group having 1-4 heteroatoms, wherein at least one heteroatom is nitrogen. In some embodiments, at least two heteroatoms are nitrogen. In some embodiments, a heterocyclyl group is directly bonded to a linkage phosphorus. In some embodiments, a heterocyclyl group is bonded to a linkage phosphorus through a linker, e.g., ═N— when the heterocyclyl group is part of a guanidine moiety who directed bonded to a linkage phosphorus through its ═N—. In some embodiments, a non-negatively charged internucleotidic linkage comprises an optionally substituted

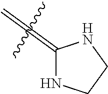 [SMILES: *=C1NCCN1]

group. In some embodiments, a non-negatively charged internucleotidic linkage comprises an substituted

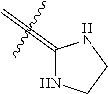 [SMILES: *=C1NCCN1]

group. In some embodiments, a non-negatively charged internucleotidic linkage comprises a

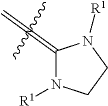

group. In some embodiments, each R¹ is independently optionally substituted C₁₋₆ alkyl. In some embodiments, each R¹ is independently methyl.

In some embodiments, a non-negatively charged internucleotidic linkage, e.g., a neutral internucleotidic linkage is not chirally controlled. In some embodiments, a non-negatively charged internucleotidic linkage is chirally controlled. In some embodiments, a non-negatively charged internucleotidic linkage is chirally controlled and its linkage phosphorus is Rp. In some embodiments, a non-negatively charged internucleotidic linkage is chirally controlled and its linkage phosphorus is Sp.

In some embodiments, an internucleotidic linkage comprises no linkage phosphorus. In some embodiments, an internucleotidic linkage has the structure of —C(O)—(O)— or —C(O)—N(R′)—, wherein R′ is as described herein. In some embodiments, an internucleotidic linkage has the structure of —C(O)—(O)—. In some embodiments, an internucleotidic linkage has the structure of —C(O)—N(R′)—, wherein R′ is as described herein. In various embodiments, —C(O)— is bonded to nitrogen. In some embodiments, an internucleotidic linkage is or comprises —C(O)—O— which is part of a carbamate moiety. In some embodiments, an internucleotidic linkage is or comprises —C(O)—O— which is part of a urea moiety.

In some embodiments, an oligonucleotide comprises 1-20, 1-15, 1-10, 1-5, or 1, 2, 3, 4, 5, 6, 7, 8, 9, 10, or more non-negatively charged internucleotidic linkages. In some embodiments, an oligonucleotide comprises 1-20, 1-15, 1-10, 1-5, or 1, 2, 3, 4, 5, 6, 7, 8, 9, 10, or more neutral internucleotidic linkages. In some embodiments, each of non-negatively charged internucleotidic linkage and/or neutral internucleotidic linkages is optionally and independently chirally controlled. In some embodiments, each non-negatively charged internucleotidic linkage in an oligonucleotide is independently a chirally controlled internucleotidic linkage. In some embodiments, each neutral internucleotidic linkage in an oligonucleotide is independently a chirally controlled internucleotidic linkage. In some embodiments, at least one non-negatively charged internucleotidic linkage/neutral internucleotidic linkage has the structure of

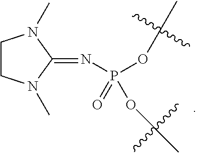 [SMILES: *OP(=O)(N=C1N(C)CCN1C)O*]

In some embodiments, an oligonucleotide comprises at least one non-negatively charged internucleotidic linkage wherein its linkage phosphorus is in Rp configuration, and at least one non-negatively charged internucleotidic linkage wherein its linkage phosphorus is in Sp configuration.

In many embodiments, as demonstrated extensively, oligonucleotides of the present disclosure comprise two or more different internucleotidic linkages. In some embodiments, an oligonucleotide comprises a phosphorothioate internucleotidic linkage and a non-negatively charged internucleotidic linkage. In some embodiments, an oligonucleotide comprises a phosphorothioate internucleotidic linkage, a non-negatively charged internucleotidic linkage, and a natural phosphate linkage. In some embodiments, a non-negatively charged internucleotidic linkage is a neutral internucleotidic linkage. In some embodiments, a non-negatively charged internucleotidic linkage is n001, n003, n004, n006, n008 or n009, n013, n020, n021, n025, n026, n029, n031, n037, n046, n047, n048, n054, or n055). In some embodiments, a non-negatively charged internucleotidic linkage is n001. In some embodiments, each phosphorothioate internucleotidic linkage is independently chirally controlled. In some embodiments, each chiral modified internucleotidic linkage is independently chirally controlled. In some embodiments, one or more non-negatively charged internucleotidic linkage are not chirally controlled.

A typical connection, as in natural DNA and RNA, is that an internucleotidic linkage forms bonds with two sugars (which can be either unmodified or modified as described herein). In many embodiments, as exemplified herein an internucleotidic linkage forms bonds through its oxygen atoms or heteroatoms with one optionally modified ribose or deoxyribose at its 5′ carbon, and the other optionally modified ribose or deoxyribose at its 3′ carbon. In some embodiments, internucleotidic linkages connect sugars that are not ribose sugars, e.g., sugars comprising N ring atoms and acyclic sugars as described herein.

In some embodiments, each nucleoside units connected by an internucleotidic linkage independently comprises a nucleobase which is independently an optionally substituted A, T, C, G, or U, or an optionally substituted tautomer of A, T, C, G or U.

In some embodiments, an oligonucleotide comprises a modified internucleotidic linkage (e.g., a modified internucleotidic linkage having the structure of Formula I, I-a, I-b, or I-c, I-n-1, I-n-2, I-n-3, I-n-4, II, II-a-1, II-a-2, II-b-1, II-b-2, II-c-1, II-c-2, II-d-1, II-d-2, etc., or a salt form thereof) as described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612 the internucleotidic linkages (e.g., those of Formula I, I-a, I-b, or I-c, I-n-1, I-n-2, I-n-3, I-n-4, II, II-a-1, II-a-2, II-b-1, II-b-2, II-c-1, II-c-2, II-d-1, II-d-2, etc.) of each of which are independently incorporated herein by reference. In some embodiments, a modified internucleotidic linkage is a non-negatively charged internucleotidic linkage. In some embodiments, provided oligonucleotides comprise one or more non-negatively charged internucleotidic linkages. In some embodiments, a non-negatively charged internucleotidic linkage is a positively charged internucleotidic linkage. In some embodiments, a non-negatively charged internucleotidic linkage is a neutral internucleotidic linkage. In some embodiments, the present disclosure provides oligonucleotides comprising one or more neutral internucleotidic linkages. In some embodiments, a non-negatively charged internucleotidic linkage or a neutral internucleotidic linkage (e.g., one of Formula I-n-1, I-n-2, I-n-3, I-n-4, II, II-a-1, II-a-2, II-b-1, II-b-2, II-c-1, II-c-2, II-d-1, II-d-2, etc.) is as described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, and/or WO 2019/032612. In some embodiments, a non-negatively charged internucleotidic linkage or neutral internucleotidic linkage is one of Formula I-n-1, I-n-2, I-n-3, I-n-4, II, II-a-1, II-a-2, II-b-1, II-b-2, II-c-1, II-c-2, II-d-1, II-d-2, etc. as described in WO 2018/223056, WO 2019/032607, WO 2019/075357, WO 2019/032607, WO 2019/075357, WO 2019/200185, WO 2019/217784, and/or WO 2019/032612, such internucleotidic linkages of each of which are independently incorporated herein by reference.

As described herein, various variables can be R, e.g., R′, R^{L}, etc. Various embodiments for R are described in the present disclosure (e.g., when describing variables that can be R). Such embodiments are generally useful for all variables that can be R. In some embodiments, R is hydrogen. In some embodiments, R is optionally substituted C₁₋₃₀ (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30) aliphatic. In some embodiments, R is optionally substituted C₁₋₂₀ aliphatic. In some embodiments, R is optionally substituted C₁₋₁₀ aliphatic. In some embodiments, R is optionally substituted C₁₋₆ aliphatic. In some embodiments, R is optionally substituted alkyl. In some embodiments, R is optionally substituted C₁₋₆ alkyl. In some embodiments, R is optionally substituted methyl. In some embodiments, R is methyl. In some embodiments, R is optionally substituted ethyl. In some embodiments, R is optionally substituted propyl. In some embodiments, R is isopropyl. In some embodiments, R is optionally substituted butyl. In some embodiments, R is optionally substituted pentyl. In some embodiments, R is optionally substituted hexyl.

In some embodiments, R is optionally substituted 3-30 membered (e.g., 3, 4, 5, 6, 7, 8, 9, 10, 11, 12, 13, 14, 15, 16, 17, 18, 19, 20, 21, 22, 23, 24, 25, 26, 27, 28, 29 or 30) cycloaliphatic. In some embodiments, R is optionally substituted cycloalkyl. In some embodiments, cycloaliphatic is monocyclic, bicyclic, or polycyclic, wherein each monocyclic unit is independently saturated or partially saturated. In some embodiments, R is optionally substituted cyclopropyl. In some embodiments, R is optionally substituted cyclobutyl. In some embodiments, R is optionally substituted cyclopentyl. In some embodiments, R is optionally substituted cyclohexyl. In some embodiments, R is optionally substituted adamantyl.

In some embodiments, R is optionally substituted C₁₋₃₀ (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30) heteroaliphatic having 1-10 heteroatoms. In some embodiments, R is optionally substituted C₁₋₂₀ aliphatic having 1-10 heteroatoms. In some embodiments, R is optionally substituted C₁₋₁₀ aliphatic having 1-10 heteroatoms. In some embodiments, R is optionally substituted C₁₋₆ aliphatic having 1-3 heteroatoms. In some embodiments, R is optionally substituted heteroalkyl. In some embodiments, R is optionally substituted C₁₋₆ heteroalkyl. In some embodiments, R is optionally substituted 3-30 membered (e.g., 3, 4, 5, 6, 7, 8, 9, 10, 11, 12, 13, 14, 15, 16, 17, 18, 19, 20, 21, 22, 23, 24, 25, 26, 27, 28, 29 or 30) heterocycloaliphatic having 1-10 heteroatoms. In some embodiments, R is optionally substituted heteroclycloalkyl. In some embodiments, heterocycloaliphatic is monocyclic, bicyclic, or polycyclic, wherein each monocyclic unit is independently saturated or partially saturated.

In some embodiments, R is optionally substituted C₆₋₃₀ aryl. In some embodiments, R is optionally substituted phenyl. In some embodiments, R is optionally substituted phenyl. In some embodiments, R is C₆₋₁₄ aryl. In some embodiments, R is optionally substituted bicyclic aryl. In some embodiments, R is optionally substituted polycyclic aryl. In some embodiments, R is optionally substituted C₆₋₃₀ arylaliphatic. In some embodiments, R is C₆₋₃₀ arylheteroaliphatic having 1-10 heteroatoms.

In some embodiments, R is optionally substituted 5-30 (5, 6, 7, 8, 9, 10, 11, 12, 13, 14, 15, 16, 17, 18, 19, 20, 21, 22, 23, 24, 25, 26, 27, 28, 29 or 30) membered heteroaryl having 1-10 heteroatoms. In some embodiments, R is optionally substituted 5-20 membered heteroaryl having 1-10 heteroatoms. In some embodiments, R is optionally substituted 5-10 membered heteroaryl having 1-10 heteroatoms. In some embodiments, R is optionally substituted 5-membered heteroaryl having 1-5 heteroatoms. In some embodiments, R is optionally substituted 5-membered heteroaryl having 1-4 heteroatoms. In some embodiments, R is optionally substituted 5-membered heteroaryl having 1-3 heteroatoms. In some embodiments, R is optionally substituted 5-membered heteroaryl having 1-2 heteroatoms. In some embodiments, R is optionally substituted 5-membered heteroaryl having one heteroatom. In some embodiments, R is optionally substituted 6-membered heteroaryl having 1-5 heteroatoms. In some embodiments, R is optionally substituted 6-membered heteroaryl having 1-4 heteroatoms. In some embodiments, R is optionally substituted 6-membered heteroaryl having 1-3 heteroatoms. In some embodiments, R is optionally substituted 6-membered heteroaryl having 1-2 heteroatoms. In some embodiments, R is optionally substituted 6-membered heteroaryl having one heteroatom. In some embodiments, R is optionally substituted monocyclic heteroaryl. In some embodiments, R is optionally substituted bicyclic heteroaryl. In some embodiments, R is optionally substituted polycyclic heteroaryl. In some embodiments, a heteroatom is nitrogen.

In some embodiments, R is optionally substituted 2-pyridinyl. In some embodiments, R is optionally substituted 3-pyridinyl. In some embodiments, R is optionally substituted 4-pyridinyl. In some embodiments, R is optionally substituted

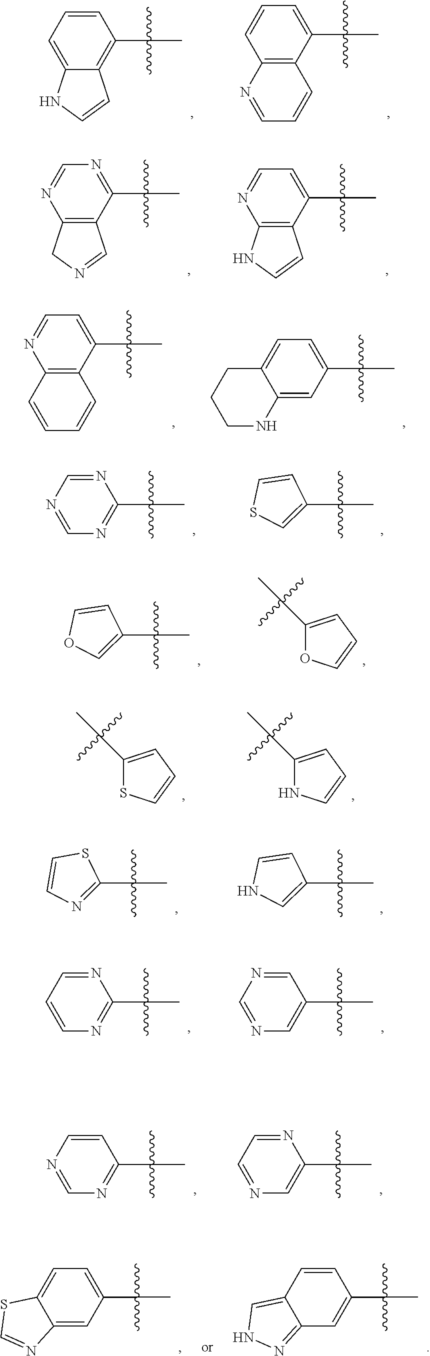

In some embodiments, R is optionally substituted 3-30 (3, 4, 5, 6, 7, 8, 9, 10, 11, 12, 13, 14, 15, 16, 17, 18, 19, 20, 21, 22, 23, 24, 25, 26, 27, 28, 29 or 30) membered heterocyclyl having 1-10 heteroatoms. In some embodiments, R is optionally substituted 3-membered heterocyclyl having 1-2 heteroatoms. In some embodiments, R is optionally substituted 4-membered heterocyclyl having 1-2 heteroatoms. In some embodiments, R is optionally substituted 5-20 membered heterocyclyl having 1-10 heteroatoms. In some embodiments, R is optionally substituted 5-10 membered heterocyclyl having 1-10 heteroatoms. In some embodiments, R is optionally substituted 5-membered heterocyclyl having 1-5 heteroatoms. In some embodiments, R is optionally substituted 5-membered heterocyclyl having 1-4 heteroatoms. In some embodiments, R is optionally substituted 5-membered heterocyclyl having 1-3 heteroatoms. In some embodiments, R is optionally substituted 5-membered heterocyclyl having 1-2 heteroatoms. In some embodiments, R is optionally substituted 5-membered heterocyclyl having one heteroatom. In some embodiments, R is optionally substituted 6-membered heterocyclyl having 1-5 heteroatoms. In some embodiments, R is optionally substituted 6-membered heterocyclyl having 1-4 heteroatoms. In some embodiments, R is optionally substituted 6-membered heterocyclyl having 1-3 heteroatoms. In some embodiments, R is optionally substituted 6-membered heterocyclyl having 1-2 heteroatoms. In some embodiments, R is optionally substituted 6-membered heterocyclyl having one heteroatom. In some embodiments, R is optionally substituted monocyclic heterocyclyl. In some embodiments, R is optionally substituted bicyclic heterocyclyl. In some embodiments, R is optionally substituted polycyclic heterocyclyl. In some embodiments, R is optionally substituted saturated heterocyclyl. In some embodiments, R is optionally substituted partially unsaturated heterocyclyl. In some embodiments, a heteroatom is nitrogen. In some embodiments R is optionally substituted

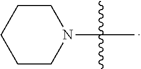 [SMILES: *N1CCCCC1]

In some embodiments, R is optionally substituted

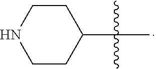 [SMILES: *C1CCNCC1]

In some embodiments, R is optionally substituted

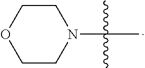 [SMILES: *N1CCOCC1]

In some embodiments, two k groups are optionally and independently taken together to form a covalent bond. In some embodiments, two or more R groups on the same atom are optionally and independently taken together with the atom to form an optionally substituted, 3-30 membered, monocyclic, bicyclic or polycyclic ring having, in addition to the atom, 0-10 heteroatoms. In some embodiments, two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

Various embodiments can comprise an optionally substituted ring, or can be taken together with their intervening atom(s) to form a ring. In some embodiments, a ring is 3-30 (e.g., 3, 4, 5, 6, 7, 8, 9, 10, 11, 12, 13, 14, 15, 16, 17, 18, 19, 20, 21, 22, 23, 24, 25, 26, 27, 28, 29 or 30) membered. In some embodiments, a ring is 3-20 membered. In some embodiments, a ring is 3-15 membered. In some embodiments, a ring is 3-10 membered. In some embodiments, a ring is 3-8 membered. In some embodiments, a ring is 3-7 membered. In some embodiments, a ring is 3-6 membered. In some embodiments, a ring is 4-20 membered. In some embodiments, a ring is 5-20 membered. In some embodiments, a ring is monocyclic. In some embodiments, a ring is bicyclic. In some embodiments, a ring is polycyclic. In some embodiments, each monocyclic ring or each monocyclic ring unit in bicyclic or polycyclic rings is independently saturated, partially saturated or aromatic. In some embodiments, each monocyclic ring or each monocyclic ring unit in bicyclic or polycyclic rings is independently 3-10 membered and has 0-5 heteroatoms.

In some embodiments, each heteroatom is independently selected oxygen, nitrogen, sulfur, silicon, and phosphorus. In some embodiments, each heteroatom is independently selected oxygen, nitrogen, sulfur, and phosphorus. In some embodiments, each heteroatom is independently selected oxygen, nitrogen, and sulfur. In some embodiments, a heteroatom is in an oxidized form.

As appreciated by those skilled in the art, many other types of internucleotidic linkages may be utilized in accordance with the present disclosure, for example, those described in U.S. Pat. Nos. 3,687,808; 4,469,863; 4,476,301; 5,177,195; 5,023,243; 5,034,506; 5,166,315; 5,185,444; 5,188,897; 5,214,134; 5,216,141; 5,235,033; 5,264,423; 5,264,564; 5,276,019; 5,278,302; 5,286,717; 5,321,131; 5,399,676; 5,405,938; 5,405,939; 5,434,257; 5,453,496; 5,455,233; 5,466,677; 5,466,677; 5,470,967; 5,476,925; 5,489,677; 5,519,126; 5,536,821; 5,541,307; 5,541,316; 5,550,111; 5,561,225; 5,563,253; 5,571,799; 5,587,361; 5,596,086; 5,602,240; 5,608,046; 5,610,289; 5,618,704; 5,623,070; 5,625,050; 5,633,360; 5,64,562; 5,663,312; 5,677,437; 5,677,439; 6,160,109; 6,239,265; 6,028,188; 6,124,445; 6,169,170; 6,172,209; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; or RE39464. In certain embodiments, a modified internucleotidic linkage is one described in U.S. Pat. No. 9,982,257, US 20170037399, US 20180216108, WO 2017192664, WO 2017015575, WO2017062862, WO 2018067973, WO 2017160741, WO 2017192679, WO 2017210647, WO 2018098264, PCT/US18/35687, PCT/US18/38835, or PCT/US18/51398, the nucleobases, sugars, internucleotidic linkages, chiral auxiliaries/reagents, and technologies for oligonucleotide synthesis (reagents, conditions, cycles, etc.) of each of which is independently incorporated herein by reference.

In certain embodiments, each internucleotidic linkage in a ds oligonucleotide is independently selected from a natural phosphate linkage, a phosphorothioate linkage, and a non-negatively charged internucleotidic linkage (e.g., n001). In certain embodiments, each internucleotidic linkage in a ds oligonucleotide is independently selected from a natural phosphate linkage, a phosphorothioate linkage, and a neutral internucleotidic linkage (e.g., n001).

In certain embodiments, a ds oligonucleotide comprises one or more nucleotides that independently comprise a phosphorus modification prone to “autorelease” under certain conditions. That is, under certain conditions, a particular phosphorus modification is designed such that it self-cleaves from the ds oligonucleotide to provide, e.g., a natural phosphate linkage. In certain embodiments, such a phosphorus modification has a structure of —O-L-R¹, wherein L is L^{B} as described herein, and R¹ is R′ as described herein. In certain embodiments, a phosphorus modification has a structure of —S-L-R¹, wherein each L and R¹ is independently as described in the present disclosure. Certain examples of such phosphorus modification groups can be found in U.S. Pat. No. 9,982,257. In certain embodiments, an autorelease group comprises a morpholino group. In certain embodiments, an autorelease group is characterized by the ability to deliver an agent to the internucleotidic phosphorus linker, which agent facilitates further modification of the phosphorus atom such as, e.g., desulfurization. In certain embodiments, the agent is water and the further modification is hydrolysis to form a natural phosphate linkage.

In certain embodiments, a ds oligonucleotide comprises one or more internucleotidic linkages that improve one or more pharmaceutical properties and/or activities of the oligonucleotide. It is well documented in the art that certain oligonucleotides are rapidly degraded by nucleases and exhibit poor cellular uptake through the cytoplasmic cell membrane (Poijarvi-Virta et al., Curr. Med. Chem. (2006), 13(28); 3441-65; Wagner et al., Med. Res. Rev. (2000), 20(6):417-51; Peyrottes et al., Mini Rev. Med. Chem. (2004), 4(4):395-408; Gosselin et al., (1996), 43(1):196-208; Bologna et al., (2002), Antisense & Nucleic Acid Drug Development 12:33-41). Vives et al. (Nucleic Acids Research (1999), 27(20):4071-76) reported that tert-butyl SATE pro-oligonucleotides displayed markedly increased cellular penetration compared to the parent oligonucleotide under certain conditions.

Double stranded oligonucleotides can comprise various number of natural phosphate linkages. In certain embodiments, 5% or more of the internucleotidic linkages of provided ds oligonucleotides are natural phosphate linkages. In certain embodiments, 10% or more of the internucleotidic linkages of provided ds oligonucleotides are natural phosphate linkages. In certain embodiments, 15% or more of the internucleotidic linkages of provided ds oligonucleotides are natural phosphate linkages. In certain embodiments, 20% or more of the internucleotidic linkages of provided ds oligonucleotides are natural phosphate linkages. In certain embodiments, 25% or more of the internucleotidic linkages of provided ds oligonucleotides are natural phosphate linkages. In certain embodiments, 30% or more of the internucleotidic linkages of provided ds oligonucleotides are natural phosphate linkages. In certain embodiments, 35% or more of the internucleotidic linkages of provided ds oligonucleotides are natural phosphate linkages. In certain embodiments, 40% or more of the internucleotidic linkages of provided ds oligonucleotides are natural phosphate linkages. In certain embodiments, provided ds oligonucleotides comprises 1, 2, 3, 4, 5, 6, 7, 8, 9, 10 or more natural phosphate linkages. In certain embodiments, provided ds oligonucleotides comprises 4, 5, 6, 7, 8, 9, 10 or more natural phosphate linkages. In certain embodiments, the number of natural phosphate linkages is 2. In certain embodiments, the number of natural phosphate linkages is 3. In certain embodiments, the number of natural phosphate linkages is 4. In certain embodiments, the number of natural phosphate linkages is 5. In certain embodiments, the number of natural phosphate linkages is 6. In certain embodiments, the number of natural phosphate linkages is 7. In certain embodiments, the number of natural phosphate linkages is 8. In certain embodiments, some or all of the natural phosphate linkages are consecutive.

In certain embodiments, the present disclosure demonstrates that, in at least some cases, Sp internucleotidic linkages, among other things, at the 5′- and/or 3′-end can improve ds oligonucleotide stability. In certain embodiments, the present disclosure demonstrates that, among other things, natural phosphate linkages and/or Rp internucleotidic linkages may improve removal of ds oligonucleotides from a system. As appreciated by a person having ordinary skill in the art, various assays known in the art can be utilized to assess such properties in accordance with the present disclosure.

In certain embodiments, each phosphorothioate internucleotidic linkage in a ds oligonucleotide or a portion thereof (e.g., a domain, a subdomain, etc.) is independently chirally controlled. In certain embodiments, each is independently Sp or Rp. In certain embodiments, a high level is Sp as described herein. In certain embodiments, each phosphorothioate internucleotidic linkage in a ds oligonucleotide or a portion thereof is chirally controlled and is Sp. In certain embodiments, one or more, e.g., about 1-5 (e.g., about 1, 2, 3, 4, or 5) is Rp.

In certain embodiments, as illustrated in certain examples, a ds oligonucleotide or a portion thereof comprises one or more non-negatively charged internucleotidic linkages, each of which is optionally and independently chirally controlled. In certain embodiments, each non-negatively charged internucleotidic linkage is independently n001. In certain embodiments, a chiral non-negatively charged internucleotidic linkage is not chirally controlled. In certain embodiments, each chiral non-negatively charged internucleotidic linkage is not chirally controlled. In certain embodiments, a chiral non-negatively charged internucleotidic linkage is chirally controlled. In certain embodiments, a chiral non-negatively charged internucleotidic linkage is chirally controlled and is Rp. In certain embodiments, a chiral non-negatively charged internucleotidic linkage is chirally controlled and is Sp. In certain embodiments, each chiral non-negatively charged internucleotidic linkage is chirally controlled. In certain embodiments, the number of non-negatively charged internucleotidic linkages in a ds oligonucleotide or a portion thereof is about 1-10, or about 1, 2, 3, 4, 5, 6, 7, 8, 9, or 10. In certain embodiments, it is about 1. In certain embodiments, it is about 2. In certain embodiments, it is about 3. In certain embodiments, it is about 4. In certain embodiments, it is about 5. In certain embodiments, it is about 6. In certain embodiments, it is about 7. In certain embodiments, it is about 8. In certain embodiments, it is about 9. In certain embodiments, it is about 10. In certain embodiments, two or more non-negatively charged internucleotidic linkages are consecutive. In certain embodiments, no two non-negatively charged internucleotidic linkages are consecutive. In certain embodiments, all non-negatively charged internucleotidic linkages in a ds oligonucleotide or a portion thereof are consecutive (e.g., 3 consecutive non-negatively charged internucleotidic linkages). In certain embodiments, a non-negatively charged internucleotidic linkage, or two or more (e.g., about 2, about 3, about 4 etc.) consecutive non-negatively charged internucleotidic linkages, are at the 3′-end of a ds oligonucleotide or a portion thereof. In certain embodiments, the last two or three or four internucleotidic linkages of a ds oligonucleotide or a portion thereof comprise at least one internucleotidic linkage that is not a non-negatively charged internucleotidic linkage.

In certain embodiments, the last two or three or four internucleotidic linkages of a ds oligonucleotide or a portion thereof comprise at least one internucleotidic linkage that is not n001. In certain embodiments, the internucleotidic linkage linking the first two nucleosides of a ds oligonucleotide or a portion thereof is a non-negatively charged internucleotidic linkage.

In certain embodiments, the internucleotidic linkage linking the last two nucleosides of a ds oligonucleotide or a portion thereof is a non-negatively charged internucleotidic linkage. In certain embodiments, the internucleotidic linkage linking the first two nucleosides of a ds oligonucleotide or a portion thereof is a phosphorothioate internucleotidic linkage. In certain embodiments, it is Sp. In certain embodiments, the internucleotidic linkage linking the last two nucleosides of a ds oligonucleotide or a portion thereof is a phosphorothioate internucleotidic linkage. In certain embodiments, it is Sp.

In certain embodiments, one or more chiral internucleotidic linkages are chirally controlled and one or more chiral internucleotidic linkages are not chirally controlled. In certain embodiments, each phosphorothioate internucleotidic linkage is independently chirally controlled, and one or more non-negatively charged internucleotidic linkage are not chirally controlled. In certain embodiments, each phosphorothioate internucleotidic linkage is independently chirally controlled, and each non-negatively charged internucleotidic linkage is not chirally controlled. In certain embodiments, the internucleotidic linkage between the first two nucleosides of a ds oligonucleotide is a non-negatively charged internucleotidic linkage.

In certain embodiments, the internucleotidic linkage between the last two nucleosides are each independently a non-negatively charged internucleotidic linkage. In certain embodiments, both are independently non-negatively charged internucleotidic linkages. In certain embodiments, each non-negatively charged internucleotidic linkage is independently neutral internucleotidic linkage. In certain embodiments, each non-negatively charged internucleotidic linkage is independently n001.

In certain embodiments, a controlled level of ds oligonucleotides in a composition are desired ds oligonucleotides. In certain embodiments, of all ds oligonucleotides in a composition that share a common base sequence (e.g., a desired sequence for a purpose), or of all ds oligonucleotides in a composition, level of desired ds oligonucleotides (which may exist in various forms (e.g., salt forms) and typically differ only at non-chirally controlled internucleotidic linkages (various forms of the same stereoisomer can be considered the same for this purpose)) is about 5%-100%, 10%-100%, 20%-100%, 30%-100%, 40%-100%, 50%-100%, 60%-100%, 70%-100%, 80-100%, 90-100%, 95-100%, 50%-90%, about 5%, 10%, 15%, 20%, 25%, 30%, 35%, 40%, 45%, 50%, 55%, 60%, 65%, 70%, 75%, 80%, 85%, 90%, 95%, or 100%, or at least 5%, 10%, 15%, 20%, 25%, 30%, 35%, 40%, 45%, 50%, 55%, 60%, 65%, 70%, 75%, 80%, 85%, 90%, 95%, or 99%. In certain embodiments, a level is at least about 50%. In certain embodiments, a level is at least about 60%. In certain embodiments, a level is at least about 70%. In certain embodiments, a level is at least about 75%. In certain embodiments, a level is at least about 80%. In certain embodiments, a level is at least about 85%. In certain embodiments, a level is at least about 90%. In certain embodiments, a level is or is at least (DS)^{nc}, wherein DS is about 90%, 91%, 92%, 93%, 94%, 95%, 96%, 97%, 98%, 99% or 99.5% and nc is the number of chirally controlled internucleotidic linkages as described in the present disclosure (e.g., 1-50, 1-40, 1-30, 1-25, 1-20, 5-50, 5-40, 5-30, 5-25, 5-20, 1, 2, 3, 4, 5, 6, 7, 8, 9, 10, 11, 12, 13, 14, 15, 16, 17, 18, 19, 20, 21, 22, 23, 24, 25 or more). In certain embodiments, a level is or is at least (DS)^{nc}, wherein DS is 95%-100%.

Various types of internucleotidic linkages may be utilized in combination of other structural elements, e.g., sugars, to achieve desired ds oligonucleotide properties and/or activities. For example, the present disclosure routinely utilizes modified internucleotidic linkages and modified sugars, optionally with natural phosphate linkages and natural sugars, in designing ds oligonucleotides. In certain embodiments, the present disclosure provides a ds oligonucleotide comprising one or more modified sugars. In certain embodiments, the present disclosure provides a ds oligonucleotide comprising one or more modified sugars and one or more modified internucleotidic linkages, one or more of which are natural phosphate linkages.

### Double Stranded Oligonucleotide Compositions

Among other things, the present disclosure provides various ds oligonucleotide compositions. In some embodiments, the present disclosure provides ds oligonucleotide compositions of ds oligonucleotides described herein. In some embodiments, a ds oligonucleotide composition, e.g., a ds oligonucleotide targeting HSD17B13 composition, comprises a plurality of an oligonucleotide described in the present disclosure. In some embodiments, a ds oligonucleotide composition, e.g., a ds oligonucleotide targeting HSD17B13 composition, is chirally controlled. In some embodiments, a ds oligonucleotide composition, e.g., a ds oligonucleotide targeting HSD17B13 composition, is not chirally controlled (stereorandom).

Linkage phosphorus of natural phosphate linkages is achiral. Linkage phosphorus of many modified internucleotidic linkages, e.g., phosphorothioate internucleotidic linkages, are chiral. In some embodiments, during preparation of oligonucleotide compositions (e.g., in traditional phosphoramidite oligonucleotide synthesis), configurations of chiral linkage phosphorus are not purposefully designed or controlled, creating non-chirally controlled (stereorandom) oligonucleotide compositions (substantially racemic preparations) which are complex, random mixtures of various stereoisomers (diastereoisomers)—for oligonucleotides with n chiral internucleotidic linkages (linkage phosphorus being chiral), typically 2ⁿ stereoisomers (e.g., when n is 10, 2¹⁰=1,032; when n is 20, 2²⁰=1,048,576). These stereoisomers have the same constitution, but differ with respect to the pattern of stereochemistry of their linkage phosphorus.

In some embodiments, stereorandom oligonucleotide compositions have sufficient properties and/or activities for certain purposes and/or applications. In some embodiments, stereorandom oligonucleotide compositions can be cheaper, easier and/or simpler to produce than chirally controlled oligonucleotide compositions. However, stereoisomers within stereorandom compositions may have different properties, activities, and/or toxicities, resulting in inconsistent therapeutic effects and/or unintended side effects by stereorandom compositions, particularly compared to certain chirally controlled oligonucleotide compositions of oligonucleotides of the same constitution.

### Chirally Controlled Double Stranded Oligonucleotide Compositions

In some embodiments, the present disclosure encompasses technologies for designing and preparing chirally controlled ds oligonucleotide compositions. In some embodiments, a chirally controlled ds oligonucleotide composition comprises a controlled/pre-determined (not random as in stereorandom compositions) level of a plurality of ds oligonucleotides, wherein the ds oligonucleotides share the same linkage phosphorus stereochemistry at one or more chiral internucleotidic linkages (chirally controlled internucleotidic linkages). In some embodiments, ds oligonucleotides of a plurality share the same pattern of backbone chiral centers (stereochemistry of linkage phosphorus). In some embodiments, a pattern of backbone chiral centers is as described in the present disclosure. In some embodiments, ds oligonucleotides of a plurality share a common constitution. In some embodiments, they are structurally identical.

For example, in some embodiments, the present disclosure provides a ds oligonucleotide composition comprising a plurality of ds oligonucleotides, wherein oligonucleotides of the plurality share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);
   wherein level of ds oligonucleotides of the plurality in the composition is non-random (e.g., controlled/pre-determined as described herein).

In some embodiments, the present disclosure provides a ds oligonucleotide composition comprising a plurality of oligonucleotides, wherein oligonucleotides of the plurality share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);
wherein the composition is enriched relative to a substantially racemic preparation of oligonucleotides sharing the common base sequence for oligonucleotides of the plurality.

In some embodiments, the present disclosure provides a ds oligonucleotide composition comprising a plurality of ds oligonucleotides, wherein ds oligonucleotides of the plurality share:
1) a common base sequence, and
2) the same linkage phosphorus stereochemistry independently at one or more (e.g., about 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more) chiral internucleotidic linkages (“chirally controlled internucleotidic linkages”);
wherein about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all ds oligonucleotides in the composition that share the common base sequence are oligonucleotides of the plurality.

In some embodiments, the percentage/level of the ds oligonucleotides of a plurality is or is at least (DS)^{nc}, wherein DS is 90%-100%, and nc is the number of chirally controlled internucleotidic linkages. In some embodiments, nc is 5, 6, 7, 8, 9, 10 or more. In some embodiments, a percentage/level is at least 10%. In some embodiments, a percentage/level is at least 20%. In some embodiments, a percentage/level is at least 30%. In some embodiments, a percentage/level is at least 40%. In some embodiments, a percentage/level is at least 50%. In some embodiments, a percentage/level is at least 60%. In some embodiments, a percentage/level is at least 65%. In some embodiments, a percentage/level is at least 70%. In some embodiments, a percentage/level is at least 75%. In some embodiments, a percentage/level is at least 80%. In some embodiments, a percentage/level is at least 85%. In some embodiments, a percentage/level is at least 90%. In some embodiments, a percentage/level is at least 95%.

In some embodiments, ds oligonucleotides of a plurality share a common pattern of backbone linkages. In some embodiments, each ds oligonucleotide of a plurality independently has an internucleotidic linkage of a particular constitution (e.g., —O—P(O)(SH)—O—) or a salt form thereof (e.g., —O—P(O)(SNa)—O—) independently at each internucleotidic linkage site. In some embodiments, internucleotidic linkages at each internucleotidic linkage site are of the same form. In some embodiments, internucleotidic linkages at each internucleotidic linkage site are of different forms.

In some embodiments, ds oligonucleotides of a plurality share a common constitution. In some embodiments, ds oligonucleotides of a plurality are of the same form of a common constitution. In some embodiments, ds oligonucleotides of a plurality are of two or more forms of a common constitution. In some embodiments, ds oligonucleotides of a plurality are each independently of a particularly ds oligonucleotide or a pharmaceutically acceptable salt thereof, or of a ds oligonucleotide having the same constitution as the particularly ds oligonucleotide or a pharmaceutically acceptable salt thereof. In some embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, 99%, or 100%, or at least 5%, 10%, 20%, 30%, 40%, 50%, 60%, 70%, 80%, 85%, 90%, 91%, 92%, 93%, 94%, 95%, 96%, 97%, 98%, or 99%) of all ds oligonucleotides in the composition that share a common constitution are oligonucleotides of the plurality. In some embodiments, a percentage of a level is or is at least (DS)^{nc}, wherein DS is 90%-100%, and nc is the number of chirally controlled internucleotidic linkages. In some embodiments, nc is 5, 6, 7, 8, 9, 10 or more. In some embodiments, a level is at least 10%. In some embodiments, a level is at least 20%. In some embodiments, a level is at least 30%. In some embodiments, a level is at least 40%. In some embodiments, a level is at least 50%. In some embodiments, a level is at least 60%. In some embodiments, a level is at least 65%. In some embodiments, a level is at least 70%. In some embodiments, a level is at least 75%. In some embodiments, a level is at least 80%. In some embodiments, a level is at least 85%. In some embodiments, a level is at least 90%. In some embodiments, a level is at least 95%.

In some embodiments, each phosphorothioate internucleotidic linkage is independently a chirally controlled internucleotidic linkage.

In some embodiments, the present disclosure provides a chirally controlled ds oligonucleotide composition comprising a plurality of oligonucleotides of a particular oligonucleotide type characterized by:
a) a common base sequence;
b) a common pattern of backbone linkages;
c) a common pattern of backbone chiral centers;
wherein the composition is enriched, relative to a substantially racemic preparation of ds oligonucleotides having the same common base sequence, for ds oligonucleotides of the particular ds oligonucleotide type.

In some embodiments, the present disclosure provides a chirally controlled ds oligonucleotide composition comprising a plurality of ds oligonucleotides of a particular ds oligonucleotide type characterized by:
a) a common base sequence;
b) a common pattern of backbone linkages;
c) a common pattern of backbone chiral centers;
wherein ds oligonucleotides of the plurality comprise at least one internucleotidic linkage comprising a common linkage phosphorus in the Sp configuration;
wherein the composition is enriched, relative to a substantially racemic preparation of oligonucleotides having the same common base sequence, for ds oligonucleotides of the particular ds oligonucleotide type.

Common patterns of backbone chiral centers, as appreciated by those skilled in the art, comprise at least one Rp or at least one Sp. Certain patterns of backbone chiral centers are illustrated in, e.g., Table 1.

In some embodiments, a chirally controlled ds oligonucleotide composition is enriched, relative to a substantially racemic preparation of ds oligonucleotides share the same common base sequence and a common pattern of backbone linkages, for oligonucleotides of the particular ds oligonucleotide type.

In some embodiments, ds oligonucleotides of a plurality, e.g., a particular ds oligonucleotide type, have a common pattern of backbone phosphorus modifications and a common pattern of nucleoside modifications. In some embodiments, ds oligonucleotides of a plurality have a common pattern of sugar modifications. In some embodiments, ds oligonucleotides of a plurality have a common pattern of base modifications. In some embodiments, ds oligonucleotides of a plurality have a common pattern of nucleoside modifications. In some embodiments, ds oligonucleotides of a plurality have the same constitution. In many embodiments, ds oligonucleotides of a plurality are identical. In some embodiments, ds oligonucleotides of a plurality are of the same oligonucleotide (as those skilled in the art will appreciate, such ds oligonucleotides may each independently exist in one of the various forms of the ds oligonucleotide, and may be the same, or different forms of the oligonucleotide). In some embodiments, ds oligonucleotides of a plurality are each independently of the same ds oligonucleotide or a pharmaceutically acceptable salt thereof.

In some embodiments, the present disclosure provides chirally controlled ds oligonucleotide compositions, e.g., of many ds oligonucleotides in Table 1 whose “stereochemistry/linkage” contain S and/or R. In some embodiments, ds oligonucleotides of a plurality are each independently a particular oligonucleotide in Table 1 whose “stereochemistry/linkage” contains S and/or R, optionally in various forms. In some embodiments, oligonucleotides of a plurality are each independently a particular oligonucleotide in Table 1 whose “stereochemistry/linkage” contains S and/or R, or a pharmaceutically acceptable salt thereof.

In some embodiments, level of a plurality of ds oligonucleotides in a composition can be determined as the product of the diastereopurity of each chirally controlled internucleotidic linkage in the oligonucleotides. In some embodiments, diastereopurity of an internucleotidic linkage connecting two nucleosides in an oligonucleotide (or nucleic acid) is represented by the diastereopurity of an internucleotidic linkage of a dimer connecting the same two nucleosides, wherein the dimer is prepared using comparable conditions, in some instances, identical synthetic cycle conditions.

In some embodiments, all chiral internucleotidic linkages are independently chiral controlled, and the composition is a completely chirally controlled oligonucleotide composition. In some embodiments, not all chiral internucleotidic linkages are chiral controlled internucleotidic linkages, and the composition is a partially chirally controlled oligonucleotide composition.

Oligonucleotides may comprise or consist of various patterns of backbone chiral centers (patterns of stereochemistry of chiral linkage phosphorus). Certain useful patterns of backbone chiral centers are described in the present disclosure. In some embodiments, a plurality of oligonucleotides share a common pattern of backbone chiral centers, which is or comprises a pattern described in the present disclosure (e.g., as in “Stereochemistry and Patterns of Backbone Chiral Centers”, a pattern of backbone chiral centers of a chirally controlled oligonucleotide in Table 1, etc.).

In some embodiments, a chirally controlled oligonucleotide composition is chirally pure (or stereopure, stereochemically pure) oligonucleotide composition, wherein the oligonucleotide composition comprises a plurality of oligonucleotides, wherein the oligonucleotides are independently of the same stereoisomer [including that each chiral element of the oligonucleotides, including each chiral linkage phosphorus, is independently defined (stereodefined)]. A chirally pure (or stereopure, stereochemically pure) oligonucleotide composition of an oligonucleotide stereoisomer does not contain other stereoisomers (as appreciated by those skilled in the art, one or more unintended stereoisomers may exist as impurities from, e.g., preparation, storage, etc.).

Chirally controlled oligonucleotide compositions can demonstrate a number of advantages over stereorandom oligonucleotide compositions. Among other things, chirally controlled oligonucleotide compositions are more uniform than corresponding stereorandom oligonucleotide compositions with respect to oligonucleotide structures. By controlling stereochemistry, compositions of individual stereoisomers can be prepared and assessed, so that chirally controlled oligonucleotide composition of stereoisomers with desired properties and/or activities can be developed. In some embodiments, chirally controlled oligonucleotide compositions provides better delivery, stability, clearance, activity, selectivity, and/or toxicity profiles compared to, e.g., corresponding stereorandom oligonucleotide compositions. In some embodiments, chirally controlled oligonucleotide compositions provide better efficacy, fewer side effects, and/or more convenient and effective dosage regimens. Among other things, patterns of backbone chiral centers as described herein can be utilized to provide controlled cleavage of oligonucleotide targets (e.g., transcripts such as pre-mRNA, mature mRNA, etc.; including control of cleavage sites, rate and/or extent of cleavage at cleavage sites, and/or overall rate and extent of cleavage, etc.).

In some embodiments, oligonucleotides in provided compositions, e.g., chirally controlled oligonucleotide compositions, are ds oligonucleotides targeting HSD17B13 as described herein.

In some embodiments, the present disclosure provides a stereorandom oligonucleotide composition, e.g., a stereorandom ds oligonucleotide targeting HSD17B13 composition. In some embodiments, the present disclosure provides a stereorandom ds oligonucleotide targeting HSD17B13 composition which is capable of decreasing the level, activity or expression of a HSD17B13 gene or a gene product thereof. In some embodiments, the present disclosure provides a stereorandom ds oligonucleotide targeting HSD17B13 composition which is capable of decreasing the level, activity or expression of a HSD17B13 gene or a gene product thereof, and wherein the base sequence of the ds oligonucleotides targeting HSD17B13 is, comprises, or comprises a span (e.g., at least 10 or 15 contiguous bases) of a base sequence disclosed herein (e.g., a base sequence in Table 1, wherein each T may be independently replaced with U and vice versa).

In some embodiments, an oligonucleotide composition comprises one or more internucleotidic linkages which are stereocontrolled (chirally controlled; in some embodiments, stereopure) and one or more internucleotidic linkages which are stereorandom. In some embodiments, a ds oligonucleotide targeting HSD17B13 composition comprises one or more internucleotidic linkages which are stereocontrolled (chirally controlled; in some embodiments, stereopure) and one or more internucleotidic linkages which are stereorandom. In some embodiments, an oligonucleotide composition comprises one or more internucleotidic linkages which are stereocontrolled (e.g., chirally controlled or stereopure) and one or more internucleotidic linkages which are stereorandom. Such oligonucleotides may target various targets and may have various base sequences, and may be capable of operating via one or more of various modalities (e.g., RNase H mechanism, steric hindrance, double- or single-stranded RNA interference, exon skipping modulation, CRISPR, aptamer, etc.).

In some embodiments, the present disclosure provides a chirally controlled oligonucleotide composition, e.g., chirally controlled ds oligonucleotide targeting HSD17B13 composition. In some embodiments, provided chirally controlled oligonucleotide compositions comprise a plurality of oligonucleotides, e.g., ds oligonucleotides targeting HSD17B13, of the same constitution, and have one or more internucleotidic linkages. In some embodiments, a plurality of oligonucleotides, e.g., in a chirally controlled oligonucleotide composition, is a plurality of an oligonucleotide selected from Table 1, wherein the oligonucleotide comprises at least one Rp or Sp linkage phosphorus in a chirally controlled internucleotidic linkage. In some embodiments, a plurality of oligonucleotides, e.g., in a chirally controlled oligonucleotide composition, is a plurality of an oligonucleotide selected from Table 1, wherein each phosphorothioate internucleotidic linkage in the oligonucleotide is independently chirally controlled (each phosphorothioate internucleotidic linkage is independently Rp or Sp). In some embodiments, an oligonucleotide composition, e.g., a ds oligonucleotide targeting HSD17B13 composition is a substantially pure preparation of a single oligonucleotide in that oligonucleotides in the composition that are not the single oligonucleotide are impurities from the preparation process of the single oligonucleotide, in some case, after certain purification procedures. In some embodiments, a single oligonucleotide is an oligonucleotide of Table 1, wherein each chiral internucleotidic linkage of the oligonucleotide is chirally controlled (e.g., indicated as S or R but not X in “Stereochemistry/Linkage”).

In some embodiments, a chirally controlled oligonucleotide composition can have, relative to a corresponding stereorandom oligonucleotide composition, increased activity and/or stability, increased delivery, and/or decreased ability to elicit adverse effects such as complement, TLR9 activation, etc. In some embodiments, a stereorandom (non-chirally controlled) oligonucleotide composition differs from a chirally controlled oligonucleotide composition in that its corresponding plurality of oligonucleotides do not contain any chirally controlled internucleotidic linkages but the stereorandom oligonucleotide composition is otherwise identical to the chirally controlled oligonucleotide composition.

In some embodiments, the present disclosure pertains to a chirally controlled ds oligonucleotide targeting HSD17B13 composition which is capable of decreasing the level, activity or expression of a HSD17B13 gene or a gene product thereof.

In some embodiments, the present disclosure provides a chirally controlled ds oligonucleotide targeting HSD17B13 composition which is capable of decreasing the level, activity or expression of a HSD17B13 gene or a gene product thereof, and comprises a plurality of oligonucleotides which share a common base sequence that is or comprises a base sequence disclosed herein (e.g., in Table 1, wherein each T may be independently replaced with U and vice versa).

In some embodiments, a provided chirally controlled oligonucleotide composition is a chirally controlled ds oligonucleotide targeting HSD17B13 composition comprising a plurality of ds oligonucleotides targeting HSD17B13. In some embodiments, a chirally controlled oligonucleotide composition is a chirally pure (or “stereochemically pure”) oligonucleotide composition. In some embodiments, the present disclosure provides a chirally pure oligonucleotide composition of an oligonucleotide in Table 1, wherein each chiral internucleotidic linkage of the oligonucleotide is independently chirally controlled (Rp or Sp, e.g., R or S but not X in “Stereochemistry/Linkage”). As one of ordinary skill in the art will understand, chemical selectivity rarely, if ever, achieves completeness (absolute 100%). In some embodiments, a chirally pure oligonucleotide composition comprises a plurality of oligonucleotides, wherein oligonucleotides of the plurality are structurally identical and all have the same structure (the same stereoisomeric form; in the context of oligonucleotide, typically the same diastereomeric form as typically multiple chiral centers exist in an oligonucleotide), and the chirally pure oligonucleotide composition does not contain any other stereoisomers (in the context of oligonucleotide, typically diastereomers as typically multiple chiral centers exist in an oligonucleotide; to the extent, e.g., achievable by stereoselective preparation). As appreciated by those skilled in the art, stereorandom (or “racemic”, “non-chirally controlled”) oligonucleotide compositions are random mixtures of many stereoisomers (e.g., 2ⁿ diastereoisomers wherein n is the number of chiral linkage phosphorus for oligonucleotides in which other chiral centers (e.g., carbon chiral centers in sugars) are chirally controlled each independently existing in one configuration and only chiral linkage phosphorus centers are not chirally controlled).

Certain data showing properties and/or activities of chirally controlled oligonucleotide composition, e.g., chirally controlled ds oligonucleotide targeting HSD17B13 compositions in decreasing the level, activity and/or expression of a HSD17B13 target gene or a gene product thereof, are shown in, for example, the Examples section of this document.

In some embodiments, the present disclosure provides an oligonucleotide composition comprising oligonucleotides that comprise at least one chiral linkage phosphorus. In some embodiments, the present disclosure provides a ds oligonucleotide targeting HSD17B13 composition comprising ds oligonucleotides targeting HSD17B13 that comprise at least one chiral linkage phosphorus. In some embodiments, the present disclosure provides a ds oligonucleotide targeting HSD17B13 composition in which the ds oligonucleotides targeting HSD17B13 comprise a chirally controlled phosphorothioate internucleotidic linkage, wherein the linkage phosphorus has a Rp configuration. In some embodiments, the present disclosure provides a ds oligonucleotide targeting HSD17B13 composition in which the ds oligonucleotides targeting HSD17B13 comprise a chirally controlled phosphorothioate internucleotidic linkage, wherein the linkage phosphorus has a Sp configuration.

In some embodiments, compared to reference oligonucleotide compositions, provided chirally controlled oligonucleotide compositions (e.g., chirally controlled ds oligonucleotide targeting HSD17B13 compositions) are surprisingly effective. In some embodiments, desired biological effects (e.g., as measured by decreased levels of mRNA, proteins, etc. whose levels are targeted for reduction) can be enhanced by more than 5, 10, 15, 20, 25, 30, 40, 50, or 100 fold (e.g., as measured by remaining levels of mRNA, proteins, etc.). In some embodiments, a change is measured by decrease of an undesired mRNA level compared to a reference condition. In some embodiments, a change is measured by increase of a desired mRNA level compared to a reference condition. In some embodiments, a change is measured by decrease of an undesired mRNA level compared to a reference condition. In some embodiments, a reference condition is absence of treatment, e.g., by a chirally controlled oligonucleotide composition. In some embodiments, a reference condition is a corresponding stereorandom composition of oligonucleotides having the same constitution.

In some embodiments, the present disclosure provides a chirally controlled oligonucleotide composition, e.g., a chirally controlled ds oligonucleotide targeting HSD17B13 composition, wherein the linkage phosphorus of at least one chirally controlled internucleotidic linkage is Sp. In some embodiments, the present disclosure provides a chirally controlled oligonucleotide composition, e.g., a chirally controlled ds oligonucleotide targeting HSD17B13 composition, wherein the majority of linkage phosphorus of chirally controlled internucleotidic linkages are Sp. In some embodiments, about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more, of all chirally controlled phosphorothioate internucleotidic linkages are Sp. In some embodiments, about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more, of all phosphorothioate internucleotidic linkages are chirally controlled and are Sp. In some embodiments, about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more, of all chirally controlled internucleotidic linkages (or of all chiral internucleotidic linkages, or of all internucleotidic linkages) are Sp. In some embodiments, the present disclosure provides a chirally controlled oligonucleotide composition, e.g., a chirally controlled ds oligonucleotide targeting HSD17B13 composition, wherein the majority of chiral internucleotidic linkages are chirally controlled and are Sp at their linkage phosphorus. In some embodiments, the present disclosure provides a chirally controlled oligonucleotide composition, e.g., a chirally controlled ds oligonucleotide targeting HSD17B13 composition, wherein each chiral internucleotidic linkage is chirally controlled and each chiral linkage phosphorus is Sp. In some embodiments, the present disclosure provides a chirally controlled oligonucleotide composition, e.g., chirally controlled ds oligonucleotide targeting HSD17B13 composition, wherein at least one chirally controlled internucleotidic linkage has a Rp linkage phosphorus. In some embodiments, the present disclosure provides a chirally controlled oligonucleotide composition, e.g., a chirally controlled ds oligonucleotide targeting HSD17B13 composition, wherein at least one chirally controlled internucleotidic linkage comprises a Rp linkage phosphorus and at least one chirally controlled internucleotidic linkage comprises a Sp linkage phosphorus. In some embodiments, at least one phosphorothioate internucleotidic linkage is chirally controlled and Rp. In some embodiments, about 1-5, e.g., about 1, 2, 3, 4, or 5 phosphorothioate internucleotidic linkage is chirally controlled and Rp. In some embodiments, about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more, of all chirally controlled non-negatively charged internucleotidic linkages (e.g., n001) are Rp. In some embodiments, each chirally controlled n001 is Rp.

### Stereochemistry and Patterns of Backbone Chiral Centers

In contrast to natural phosphate linkages, linkage phosphorus of chiral modified internucleotidic linkages, e.g., phosphorothioate internucleotidic linkages, are chiral. Among other things, the present disclosure provides technologies (e.g., oligonucleotides, compositions, methods, etc.) comprising control of stereochemistry of chiral linkage phosphorus in chiral internucleotidic linkages. In some embodiments, as demonstrated herein, control of stereochemistry can provide improved properties and/or activities, including desired stability, reduced toxicity, improved reduction of target nucleic acids, etc. In some embodiments, the present disclosure provides useful patterns of backbone chiral centers for oligonucleotides and/or regions thereof, which pattern is a combination of stereochemistry of each chiral linkage phosphorus (Rp or Sp) of chiral linkage phosphorus, indication of each achiral linkage phosphorus (Op, if any), etc. from 5′ to 3′. In some embodiments, patterns of backbone chiral centers can control cleavage patterns of target nucleic acids when they are contacted with provided oligonucleotides or compositions thereof in a cleavage system (e.g., in vitro assay, cells, tissues, organs, organisms, subjects, etc.). In some embodiments, patterns of backbone chiral centers improve cleavage efficiency and/or selectivity of target nucleic acids when they are contacted with provided oligonucleotides or compositions thereof in a cleavage system.

In some embodiments, a pattern of backbone chiral centers of an oligonucleotide or a region thereof comprises or is any (Np)n(Op)m, wherein Np is Rp or Sp, Op represents a linkage phosphorus being achiral (e.g., as for the linkage phosphorus of natural phosphate linkages), and each of n and m is independently as defined and described in the present disclosure. In some embodiments, a pattern of backbone chiral centers of an oligonucleotide or a region thereof comprises or is (Sp)n(Op)m, wherein each variable is independently as defined and described in the present disclosure. In some embodiments, a pattern of backbone chiral centers of an oligonucleotide or a region thereof comprises or is (Rp)n(Op)m, wherein each variable is independently as defined and described in the present disclosure. In some embodiments, n is 1. In some embodiments, a pattern of backbone chiral centers of an oligonucleotide or a region thereof comprises or is (Sp)(Op)m, wherein m is 1, 2, 3, 4, 5, 6, 7, 8, 9, or 10. In some embodiments, a pattern of backbone chiral centers of an oligonucleotide or a region thereof comprises or is (Rp)(Op)m, wherein m is 1, 2, 3, 4, 5, 6, 7, 8, 9, or 10. In some embodiments, the pattern of backbone chiral centers of a 5′-wing is or comprises (Np)n(Op)m. In some embodiments, the pattern of backbone chiral centers of a 5′-wing is or comprises (Sp)n(Op)m. In some embodiments, the pattern of backbone chiral centers of a 5′-wing is or comprises (Rp)n(Op)m. In some embodiments, the pattern of backbone chiral centers of a 5′-wing is or comprises (Sp)(Op)m. In some embodiments, the pattern of backbone chiral centers of a 5′-wing is or comprises (Rp)(Op)m. In some embodiments, the pattern of backbone chiral centers of a 5′-wing is (Sp)(Op)m. In some embodiments, the pattern of backbone chiral centers of a 5′-wing is (Rp)(Op)m. In some embodiments, the pattern of backbone chiral centers of a 5′-wing is (Sp)(Op)m, wherein Sp is the linkage phosphorus configuration of the first internucleotidic linkage of the oligonucleotide from the 5′-end. In some embodiments, the pattern of backbone chiral centers of a 5′-wing is (Rp)(Op)m, wherein Rp is the linkage phosphorus configuration of the first internucleotidic linkage of the oligonucleotide from the 5′-end. In some embodiments, as described in the present disclosure, m is 2; in some embodiments, m is 3; in some embodiments, m is 4; in some embodiments, m is 5; in some embodiments, m is 6.

In some embodiments, a pattern of backbone chiral centers of an oligonucleotide or a region thereof comprises or is (Op)m(Np)n, wherein Np is Rp or Sp, Op represents a linkage phosphorus being achiral (e.g., as for the linkage phosphorus of natural phosphate linkages), and each of n and m is independently as defined and described in the present disclosure. In some embodiments, a pattern of backbone chiral centers of an oligonucleotide or a region thereof comprises or is (Op)m(Sp)n, wherein each variable is independently as defined and described in the present disclosure. In some embodiments, a pattern of backbone chiral centers of an oligonucleotide or a region thereof comprises or is (Op)m(Rp)n, wherein each variable is independently as defined and described in the present disclosure. In some embodiments, n is 1. In some embodiments, a pattern of backbone chiral centers of an oligonucleotide or a region thereof comprises or is (Op)m(Sp), wherein m is 1, 2, 3, 4, 5, 6, 7, 8, 9, or 10. In some embodiments, a pattern of backbone chiral centers of an oligonucleotide or a region thereof comprises or is (Op)m(Rp), wherein m is 1, 2, 3, 4, 5, 6, 7, 8, 9, or 10. In some embodiments, the pattern of backbone chiral centers of a 3′-wing is or comprises (Op)m(Np)n. In some embodiments, the pattern of backbone chiral centers of a 3′-wing is or comprises (Op)m(Sp)n. In some embodiments, the pattern of backbone chiral centers of a 3′-wing is or comprises (Op)m(Rp)n. In some embodiments, the pattern of backbone chiral centers of a 3′-wing is or comprises (Op)m(Sp). In some embodiments, the pattern of backbone chiral centers of a 3′-wing is or comprises (Op)m(Rp). In some embodiments, the pattern of backbone chiral centers of a 3′-wing is (Op)m(Sp). In some embodiments, the pattern of backbone chiral centers of a 3′-wing is (Op)m(Rp). In some embodiments, the pattern of backbone chiral centers of a 3′-wing is (Op)m(Sp), wherein Sp is the linkage phosphorus configuration of the last internucleotidic linkage of the oligonucleotide from the 5′-end. In some embodiments, the pattern of backbone chiral centers of a 3′-wing is (Op)m(Rp), wherein Rp is the linkage phosphorus configuration of the last internucleotidic linkage of the oligonucleotide from the 5′-end. In some embodiments, as described in the present disclosure, m is 2; in some embodiments, m is 3; in some embodiments, m is 4; in some embodiments, m is 5; in some embodiments, m is 6.

In some embodiments, a pattern of backbone chiral centers of an oligonucleotide or a region thereof (e.g., a core) comprises or is (Sp)m(Rp/Op)n or (Rp/Op)n(Sp)m, wherein each variable is independently as described in the present disclosure. In some embodiments, a pattern of backbone chiral centers of an oligonucleotide or a region thereof (e.g., a core) comprises or is (Sp)m(Rp)n or (Rp)n(Sp)m, wherein each variable is independently as described in the present disclosure. In some embodiments, a pattern of backbone chiral centers of an oligonucleotide or a region thereof (e.g., a core) comprises or is (Sp)m(Op)n or (Op)n(Sp)m, wherein each variable is independently as described in the present disclosure. In some embodiments, a pattern of backbone chiral centers of an oligonucleotide or a region thereof (e.g., a core) comprises or is (Np)t[(Rp/Op)n(Sp)m]y or [(Rp/Op)n(Sp)m]y(Np)t, wherein y is 1-50, and each other variable is independently as described in the present disclosure. In some embodiments, a pattern of backbone chiral centers of an oligonucleotide or a region thereof (e.g., a core) comprises or is (Np)t[(Rp)n(Sp)m]y or [(Rp)n(Sp)m]y(Np)t, wherein each variable is independently as described in the present disclosure. In some embodiments, a pattern of backbone chiral centers of an oligonucleotide or a region thereof (e.g., a core) comprises or is [(Rp/Op)n(Sp)m]y(Rp)k, [(Rp/Op)n(Sp)m]y, (Sp)t[(Rp/Op)n(Sp)m]y, (Sp)t[(Rp/Op)n(Sp)m]y(Rp)k, wherein k is 1-50, and each other variable is independently as described in the present disclosure. In some embodiments, a pattern of backbone chiral centers of an oligonucleotide or a region thereof (e.g., a core) comprises or is [(Op)n(Sp)m]y(Rp)k, [(Op)n(Sp)m]y, (Sp)t[(Op)n(Sp)m]y, (Sp)t[(Op)n(Sp)m]y(Rp)k, wherein each variable is independently as described in the present disclosure. In some embodiments, a pattern of backbone chiral centers of an oligonucleotide or a region thereof (e.g., a core) comprises or is [(Rp)n(Sp)m]y(Rp)k, [(Rp)n(Sp)m]y, (Sp)t[(Rp)n(Sp)m]y, (Sp)t[(Rp)n(Sp)m]y(Rp)k, wherein each variable is independently as described in the present disclosure. In some embodiments, an oligonucleotide comprises a core region. In some embodiments, an oligonucleotide comprises a core region, wherein each sugar in the core region does not contain a 2′-OR′, wherein R¹ is as described in the present disclosure. In some embodiments, an oligonucleotide comprises a core region, wherein each sugar in the core region is independently a natural DNA sugar. In some embodiments, the pattern of backbone chiral centers of the core comprises or is (Rp)(Sp)m. In some embodiments, the pattern of backbone chiral centers of the core comprises or is (Op)(Sp)m. In some embodiments, the pattern of backbone chiral centers of the core comprises or is (Np)t[(Rp/Op)n(Sp)m]y or [(Rp/Op)n(Sp)m]y(Np)t. In some embodiments, the pattern of backbone chiral centers of the core comprises or is (Np)t[(Rp/Op)n(Sp)m]y or [(Rp/Op)n(Sp)m]y(Np)t. In some embodiments, the pattern of backbone chiral centers of the core comprises or is (Np)t[(Rp)n(Sp)m]y or [(Rp)n(Sp)m]y(Np)t. In some embodiments, the pattern of backbone chiral centers of a core comprises or is [(Rp/Op)n(Sp)m]y(Rp)k, [(Rp/Op)n(Sp)m]y, (Sp)t[(Rp/Op)n(Sp)m]y, (Sp)t[(Rp/Op)n(Sp)m]y(Rp)k. In some embodiments, a pattern of backbone chiral centers of a core comprises or is [(Op)n(Sp)m]y(Rp)k, [(Op)n(Sp)m]y, (Sp)t[(Op)n(Sp)m]y, (Sp)t[(Op)n(Sp)m]y(Rp)k. In some embodiments, a pattern of backbone chiral centers of a core comprises or is [(Rp)n(Sp)m]y(Rp)k, [(Rp)n(Sp)m]y, (Sp)t[(Rp)n(Sp)m]y, or (Sp)t[(Rp)n(Sp)m]y(Rp)k. In some embodiments, a pattern of backbone chiral centers of a core comprises [(Rp)n(Sp)m]y(Rp)k. In some embodiments, a pattern of backbone chiral centers of a core comprises [(Rp)n(Sp)m]y(Rp). In some embodiments, a pattern of backbone chiral centers of a core comprises [(Rp)n(Sp)m]y. In some embodiments, a pattern of backbone chiral centers of a core comprises (Sp)t[(Rp)n(Sp)m]y. In some embodiments, a pattern of backbone chiral centers of a core comprises (Sp)t[(Rp)n(Sp)m]y(Rp)k. In some embodiments, a pattern of backbone chiral centers of a core comprises (Sp)t[(Rp)n(Sp)m]y(Rp). In some embodiments, a pattern of backbone chiral centers of a core is [(Rp)n(Sp)m]y(Rp)k. In some embodiments, a pattern of backbone chiral centers of a core is [(Rp)n(Sp)m]y(Rp). In some embodiments, a pattern of backbone chiral centers of a core is [(Rp)n(Sp)m]y. In some embodiments, a pattern of backbone chiral centers of a core is (Sp)t[(Rp)n(Sp)m]y. In some embodiments, a pattern of backbone chiral centers of a core is (Sp)t[(Rp)n(Sp)m]y(Rp)k. In some embodiments, a pattern of backbone chiral centers of a core is (Sp)t[(Rp)n(Sp)m]y(Rp). In some embodiments, each n is 1. In some embodiments, each t is 1. In some embodiments, t is 2, 3, 4, 5, 6, 7, 8, 9, or 10. In some embodiments, each of t and n is 1. In some embodiments, each m is 2 or more. In some embodiments, k is 1. In some embodiments, k is 2-10.

In some embodiments, a pattern of backbone chiral centers comprises or is (Sp)m(Rp)n, (Rp)n(Sp)m, (Np)t(Rp)n(Sp)m, (Sp)t(Rp)n(Sp)m, (Np)t[(Rp)n(Sp)m]2, (Sp)t[(Rp)n(Sp)m]2, (Np)t(Op)n(Sp)m, (Sp)t(Op)n(Sp)m, (Np)t[(Op)n(Sp)m]2, or (Sp)t[(Op)n(Sp)m]2. In some embodiments, a pattern is (Np)t(Op/Rp)n(Sp)m(Op/Rp)n(Sp)m. In some embodiments, a pattern is (Np)t(Op/Rp)n(Sp)1-5(Op/Rp)n(Sp)m. In some embodiments, a pattern is (Np)t(Op/Rp)n(Sp)2-5(Op/Rp)n(Sp)m. In some embodiments, a pattern is (Np)t(Op/Rp)n(Sp)2(Op/Rp)n(Sp)m. In some embodiments, a pattern is (Np)t(Op/Rp)n(Sp)3(Op/Rp)n(Sp)m. In some embodiments, a pattern is (Np)t(Op/Rp)n(Sp)4(Op/Rp)n(Sp)m. In some embodiments, a pattern is (Np)t(Op/Rp)n(Sp)5(Op/Rp)n(Sp)m.

In some embodiments, Np is Sp. In some embodiments, (Op/Rp) is Op. In some embodiments, (Op/Rp) is Rp. In some embodiments, Np is Sp and (Op/Rp) is Rp. In some embodiments, Np is Sp and (Op/Rp) is Op. In some embodiments, Np is Sp and at least one (Op/Rp) is Rp, and at least one (Op/Rp) is Op. In some embodiments, a pattern of backbone chiral centers comprises or is (Rp)n(Sp)m, (Np)t(Rp)n(Sp)m, or (Sp)t(Rp)n(Sp)m, wherein m>2. In some embodiments, a pattern of backbone chiral centers comprises or is (Rp)n(Sp)m, (Np)t(Rp)n(Sp)m, or (Sp)t(Rp)n(Sp)m, wherein n is 1, at least one t>1, and at least one m>2. In some embodiments, oligonucleotides comprising core regions whose patterns of backbone chiral centers starting with Rp can provide high activities and/or improved properties. In some embodiments, oligonucleotides comprising core regions whose patterns of backbone chiral centers ending with Rp can provide high activities and/or improved properties. In some embodiments, oligonucleotides comprising core regions whose patterns of backbone chiral centers starting with Rp provide high activities (e.g., target cleavage) without significantly impacting its properties, e.g., stability. In some embodiments, oligonucleotides comprising core regions whose patterns of backbone chiral centers ending with Rp provide high activities (e.g., target cleavage) without significantly impacting its properties, e.g., stability. In some embodiments, patterns of backbone chiral centers start with Rp and end with Sp. In some embodiments, patterns of backbone chiral centers start with Rp and end with Rp. In some embodiments, patterns of backbone chiral centers start with Sp and end with Rp. Typically, for patterns of backbone chiral centers internucleotidic linkages connecting core nucleosides and wing nucleosides are included in the patterns of the core regions. In many embodiments as described in the present disclosure (e.g., various oligonucleotides in Table 1), the wing sugar connected by such a connecting internucleotidic linkage has a different structure than the core sugar connected by the same connecting internucleotidic linkage (e.g., in some embodiments, the wing sugar comprises a 2′-modification while the core sugar does not contain the same 2′-modification or have two —H at the 2′ position). In some embodiments, the wing sugar comprises a sugar modification that the core sugar does not contain. In some embodiments, the wing sugar is a modified sugar while the core sugar is a natural DNA sugar. In some embodiments, the wing sugar comprises a sugar modification at the 2′ position (less than two —H at the 2′ position), and the core sugar has no modification at the 2′-position (two —H at the 2′ position).

In some embodiments, as demonstrated herein, an additional Rp internucleotidic linkage links a sugar containing no 2′-substituent (e.g., a core sugar) and a sugar comprising a 2′-modification (e.g., 2′-OR′, wherein R′ is optionally substituted C₁₋₆ aliphatic (e.g., 2′-OMe, 2′-MOE, etc.), which can be a wing sugar). In some embodiments, an internucleotidic linkage linking a sugar containing no 2′-substituent to the 5′-end (e.g., to the 3′-carbon of the sugar) and a sugar comprising a 2′-modification to the 3′-end (e.g., to the 5′-carbon of the sugar) is a Rp internucleotidic linkage. In some embodiments, an internucleotidic linkage linking a sugar containing no 2′-substituent to the 3′-end (e.g., to the 5′-carbon of the sugar) and a sugar comprising a 2′-modification to the 5′-end (e.g., to the 3′-carbon of the sugar) is a Rp internucleotidic linkage. In some embodiments, each internucleotidic linkage linking a sugar containing no 2′-substituent and a sugar comprising a 2′-modification is independently a Rp internucleotidic linkage. In some embodiments, a Rp internucleotidic linkage is a Rp phosphorothioate internucleotidic linkage.

In some embodiments, a pattern of backbone chiral centers of a ds oligonucleotide targeting HSD17B13 or a region thereof (e.g., a core) comprises or is (Op)[(Rp/Op)n(Sp)m]y(Rp)k(Op), (Op)[(Rp/Op)n(Sp)m]y(Op), (Op)(Sp)t[(Rp/Op)n(Sp)m]y(Op), or (Op)(Sp)t[(Rp/Op)n(Sp)m]y(Rp)k(Op), wherein k is 1-50, and each other variable is independently as described in the present disclosure. In some embodiments, a pattern of backbone chiral centers of a ds oligonucleotide targeting HSD17B13 comprises or is (Op)[(Rp/Op)n(Sp)m]y(Rp)k(Op), (Op)[(Rp/Op)n(Sp)m]y(Op), (Op)(Sp)t[(Rp/Op)n(Sp)m]y(Op), or (Op)(Sp)t[(Rp/Op)n(Sp)m]y(Rp)k(Op), wherein each of f, g, h and j is independently 1-50, and each other variable is independently as described in the present disclosure, and the oligonucleotide comprises a core region whose pattern of backbone chiral centers comprises or is [(Rp/Op)n(Sp)m]y(Rp)k, [(Rp/Op)n(Sp)m]y, (Sp)t[(Rp/Op)n(Sp)m]y, or (Sp)t[(Rp/Op)n(Sp)m]y(Rp)k as described in the present disclosure. In some embodiments, a pattern of backbone chiral centers is or comprises (Op)[(Rp/Op)n(Sp)m]y(Rp)k(Op). In some embodiments, a pattern of backbone chiral centers is or comprises (Op)[(Rp/Op)n(Sp)m]y(Rp)(Op). In some embodiments, a pattern of backbone chiral centers is or comprises (Op)[(Rp/Op)n(Sp)m]y(Op). In some embodiments, a pattern of backbone chiral centers is or comprises (Op)(Sp)t[(Rp/Op)n(Sp)m]y(Op). In some embodiments, a pattern of backbone chiral centers is or comprises (Op)(Sp)t[(Rp/Op)n(Sp)m]y(Rp)k(Op). In some embodiments, a pattern of backbone chiral centers is or comprises (Op)(Sp)t[(Rp/Op)n(Sp)m]y(Rp)(Op). In some embodiments, a pattern of backbone chiral centers is or comprises (Op)[(Rp)n(Sp)m]y(Rp)k(Op). In some embodiments, a pattern of backbone chiral centers is or comprises (Op)[(Rp)n(Sp)m]y(Rp)(Op). In some embodiments, a pattern of backbone chiral centers is or comprises (Op)[(Rp)n(Sp)m]y(Op). In some embodiments, a pattern of backbone chiral centers is or comprises (Op)(Sp)t[(Rp)n(Sp)m]y(Op). In some embodiments, a pattern of backbone chiral centers is or comprises (Op)(Sp)t[(Rp)n(Sp)m]y(Rp)k(Op). In some embodiments, a pattern of backbone chiral centers is or comprises (Op)(Sp)t[(Rp)n(Sp)m]y(Rp)(Op). In some embodiments, each n is 1. In some embodiments, k is 1. In some embodiments, k is 2-10.

In some embodiments, a pattern of backbone chiral centers of a ds oligonucleotide targeting HSD17B13 or a region thereof (e.g., a core) comprises or is (Np)f(Op)g[(Rp/Op)n(Sp)m]y(Rp)k(Op)h(Np)j, (Np)f(Op)g[(Rp/Op)n(Sp)m]y(Op)h(Np)j, (Np)f(Op)g(Sp)t[(Rp/Op)n(Sp)m]y(Op)h(Np)j, or (Np)f(Op)g(Sp)t[(Rp/Op)n(Sp)m]y(Rp)k(Op)h(Np)j, wherein each of f, g, h and j is independently 1-50, and each other variable is independently as described in the present disclosure. In some embodiments, a pattern of backbone chiral centers of a ds oligonucleotide targeting HSD17B13 comprises or is (Np)f(Op)g[(Rp/Op)n(Sp)m]y(Rp)k(Op)h(Np)j, (Np)f(Op)g[(Rp/Op)n(Sp)m]y(Op)h(Np)j, (Np)f(Op)g(Sp)t[(Rp/Op)n(Sp)m]y(Op)h(Np)j, or (Np)f(Op)g(Sp)t[(Rp/Op)n(Sp)m]y(Rp)k(Op)h(Np)j, and the oligonucleotide comprises a core region whose pattern of backbone chiral centers comprises or is [(Rp/Op)n(Sp)m]y(Rp)k, [(Rp/Op)n(Sp)m]y, (Sp)t[(Rp/Op)n(Sp)m]y, or (Sp)t[(Rp/Op)n(Sp)m]y(Rp)k as described in the present disclosure. In some embodiments, a pattern of backbone chiral centers of a ds oligonucleotide targeting HSD17B13 is (Np)f(Op)g[(Rp/Op)n(Sp)m]y(Rp)k(Op)h(Np)j, (Np)f(Op)g[(Rp/Op)n(Sp)m]y(Op)h(Np)j, (Np)f(Op)g(Sp)t[(Rp/Op)n(Sp)m]y(Op)h(Np)j, or (Np)f(Op)g(Sp)t[(Rp/Op)n(Sp)m]y(Rp)k(Op)h(Np)j, and the oligonucleotide comprises a core region whose pattern of backbone chiral centers comprises or is [(Rp/Op)n(Sp)m]y(Rp)k, [(Rp/Op)n(Sp)m]y, (Sp)t[(Rp/Op)n(Sp)m]y, or (Sp)t[(Rp/Op)n(Sp)m]y(Rp)k as described in the present disclosure. In some embodiments, a pattern of backbone chiral centers is or comprises (Np)f(Op)g[(Rp/Op)n(Sp)m]y(Rp)k(Op)h(Np)j. In some embodiments, a pattern of backbone chiral centers is or comprises (Np)f(Op)g[(Rp/Op)n(Sp)m]y(Rp)(Op)h(Np)j. In some embodiments, a pattern of backbone chiral centers is or comprises (Np)f(Op)g[(Rp/Op)n(Sp)m]y(Op)h(Np)j. In some embodiments, a pattern of backbone chiral centers is or comprises (Np)f(Op)g(Sp)t[(Rp/Op)n(Sp)m]y(Op)h(Np)j. In some embodiments, a pattern of backbone chiral centers is or comprises (Np)f(Op)g(Sp)t[(Rp/Op)n(Sp)m]y(Rp)k(Op)h(Np)j. In some embodiments, a pattern of backbone chiral centers is or comprises (Np)f(Op)g(Sp)t[(Rp/Op)n(Sp)m]y(Rp)(Op)h(Np)j. In some embodiments, a pattern of backbone chiral centers is or comprises (Np)f(Op)g[(Rp)n(Sp)m]y(Rp)k(Op)h(Np)j. In some embodiments, a pattern of backbone chiral centers is or comprises (Np)f(Op)g[(Rp)n(Sp)m]y(Rp)(Op)h(Np)j. In some embodiments, a pattern of backbone chiral centers is or comprises (Np)f(Op)g[(Rp)n(Sp)m]y(Op)h(Np)j. In some embodiments, a pattern of backbone chiral centers is or comprises (Np)f(Op)g(Sp)t[(Rp)n(Sp)m]y(Op)h(Np)j. In some embodiments, a pattern of backbone chiral centers is or comprises (Np)f(Op)g(Sp)t[(Rp)n(Sp)m]y(Rp)k(Op)h(Np)j. In some embodiments, a pattern of backbone chiral centers is or comprises (Np)f(Op)g(Sp)t[(Rp)n(Sp)m]y(Rp)(Op)h(Np)j. In some embodiments, at least one Np is Sp. In some embodiments, at least one Np is Rp. In some embodiments, the 5′ most Np is Sp. In some embodiments, the 3′ most Np is Sp. In some embodiments, each Np is Sp. In some embodiments, (Np)f(Op)g[(Rp/Op)n(Sp)m]y(Rp)k(Op)h(Np)j is (Sp)(Op)g[(Rp)n(Sp)m]y(Rp)k(Op)h(Sp). In some embodiments, (Np)f(Op)g[(Rp/Op)n(Sp)m]y(Rp)k(Op)h(Np)j is (Sp)(Op)g[(Rp)n(Sp)m]y(Rp)(Op)h(Sp). In some embodiments, a pattern of backbone chiral center of an oligonucleotide is or comprises (Sp)(Op)g[(Rp)n(Sp)m]y(Rp)(Op)h(Sp). In some embodiments, a pattern of backbone chiral center of an oligonucleotide is (Sp)(Op)g[(Rp)n(Sp)m]y(Rp)(Op)h(Sp). In some embodiments, (Np)f(Op)g[(Rp/Op)n(Sp)m]y(Op)h(Np)j is (Sp)(Op)g[(Rp)n(Sp)m]y(Op)h(Sp). In some embodiments, a pattern of backbone chiral center of an oligonucleotide is or comprises (Sp)(Op)g[(Rp)n(Sp)m]y(Op)h(Sp). In some embodiments, a pattern of backbone chiral center of an oligonucleotide is (Sp)(Op)g[(Rp)n(Sp)m]y(Op)h(Sp). In some embodiments, (Np)f(Op)g(Sp)t[(Rp/Op)n(Sp)m]y(Op)h(Np)j is (Sp)(Op)g(Sp)t[(Rp)n(Sp)m]y(Op)h(Sp). In some embodiments, a pattern of backbone chiral center of an oligonucleotide is or comprises (Sp)(Op)g(Sp)t[(Rp)n(Sp)m]y(Op)h(Sp). In some embodiments, a pattern of backbone chiral center of an oligonucleotide is (Sp)(Op)g(Sp)t[(Rp)n(Sp)m]y(Op)h(Sp). In some embodiments, (Np)f(Op)g(Sp)t[(Rp/Op)n(Sp)m]y(Rp)k(Op)h(Np)j is (Sp)(Op)g(Sp)t[(Rp)n(Sp)m]y(Rp)k(Op)h(Sp). In some embodiments, (Np)f(Op)g(Sp)t[(Rp/Op)n(Sp)m]y(Rp)k(Op)h(Np)j is (Sp)(Op)g(Sp)t[(Rp)n(Sp)m]y(Rp)(Op)h(Sp). In some embodiments, a pattern of backbone chiral center of an oligonucleotide is or comprises (Sp)(Op)g(Sp)t[(Rp)n(Sp)m]y(Rp)(Op)h(Sp). In some embodiments, a pattern of backbone chiral center of an oligonucleotide is (Sp)(Op)g(Sp)t[(Rp)n(Sp)m]y(Rp)(Op)h(Sp). In some embodiments, each n is 1. In some embodiments, f is 1. In some embodiments, g is 1. In some embodiments, g is greater than 1. In some embodiments, g is 2. In some embodiments, g is 3. In some embodiments, g is 4. In some embodiments, g is 5. In some embodiments, g is 6. In some embodiments, g is 7. In some embodiments, g is 8. In some embodiments, g is 9. In some embodiments, g is 10. In some embodiments, h is 1. In some embodiments, h is greater than 1. In some embodiments, h is 2. In some embodiments, h is 3. In some embodiments, h is 4. In some embodiments, h is 5. In some embodiments, h is 6. In some embodiments, h is 7. In some embodiments, h is 8. In some embodiments, h is 9. In some embodiments, h is 10. In some embodiments, j is 1. In some embodiments, k is 1. In some embodiments, k is 2-10. In some embodiments, a pattern of backbone chiral centers of a ds oligonucleotide targeting HSD17B13 or a region thereof (e.g., a core) comprises or is [(Rp/Op)n(Sp)m]y, (Sp)t[(Rp/Op)n(Sp)m]y, (Sp)t[(Rp/Op)n(Sp)m]yRp, [(Rp/Op)n(Sp)m]y(Rp)k, (Sp)t[(Rp/Op)n(Sp)m]y(Rp)k, (Sp)t[(Rp/Op)n(Sp)m]y(Rp)k(Op)h, (Sp)t[(Rp/Op)n(Sp)m]y(Rp)k(Op)h(Np)j, wherein each variable is independently as described in the present disclosure.

In some embodiments, in a provided pattern of backbone chiral centers, at least one (Rp/Op) is Rp. In some embodiments, at least one (Rp/Op) is Op. In some embodiments, each (Rp/Op) is Rp. In some embodiments, each (Rp/Op) is Op. In some embodiments, at least one of [(Rp)n(Sp)m]y or [(Rp/Op)n(Sp)m]y of a pattern is RpSp. In some embodiments, at least one of [(Rp)n(Sp)m]y or [(Rp/Op)n(Sp)m]y of a pattern is or comprises RpSpSp. In some embodiments, at least one of [(Rp)n(Sp)m]y or [(Rp/Op)n(Sp)m]y in a pattern is RpSp, and at least one of [(Rp)n(Sp)m]y or [(Rp/Op)n(Sp)m]y in a pattern is or comprises RpSpSp. For example, in some embodiments, [(Rp)n(Sp)m]y in a pattern is (RpSp)[(Rp)n(Sp)m]_{(y-1)}; in some embodiments, [(Rp)n(Sp)m]y in a pattern is (RpSp)[RpSpSp(Sp)₍ₘ₋₂₎][(Rp)n(Sp)m]_{(y-2)}. In some embodiments, (Sp)t[(Rp)n(Sp)m]y(Rp) is (Sp)t(RpSp)[(Rp)n(Sp)m]_{(y-1)}(Rp). In some embodiments, (Sp)t[(Rp)n(Sp)m]y(Rp) is (Sp)t(RpSp)[RpSpSp(Sp)₍ₘ₋₂₎][(Rp)n(Sp)m]_{(y-2)}(Rp). In some embodiments, each [(Rp/Op)n(Sp)m] is independently [Rp(Sp)m]. In some embodiments, the first Sp of (Sp)t represents linkage phosphorus stereochemistry of the first internucleotidic linkage of an oligonucleotide from 5′ to 3′. In some embodiments, the first Sp of (Sp)t represents linkage phosphorus stereochemistry of the first internucleotidic linkage of a region from 5′ to 3′, e.g., a core. In some embodiments, the last Np of (Np)j represents linkage phosphorus stereochemistry of the last internucleotidic linkage of the oligonucleotide from 5′ to 3′. In some embodiments, the last Np is Sp.

In some embodiments, a pattern of backbone chiral centers of an oligonucleotide or a region (e.g., of a 5′-wing) is or comprises Sp(Op)₃. In some embodiments, a pattern of backbone chiral centers of an oligonucleotide or a region (e.g., of a 5′-wing) is or comprises Rp(Op)₃. In some embodiments, a pattern of backbone chiral centers of an oligonucleotide or a region (e.g., of a 3′-wing) is or comprises (Op)₃Sp. In some embodiments, a pattern of backbone chiral centers of an oligonucleotide or a region (e.g., of a 3′-wing) is or comprises (Op)₃Rp. In some embodiments, a pattern of backbone chiral centers of an oligonucleotide or a region (e.g., of a core) is or comprises Rp(Sp)₄Rp(Sp)₄Rp. In some embodiments, a pattern of backbone chiral centers of an oligonucleotide or a region (e.g., of a core) is or comprises (Sp)₅Rp(Sp)₄Rp. In some embodiments, a pattern of backbone chiral centers of an oligonucleotide or a region (e.g., of a core) is or comprises (Sp)₅Rp(Sp)₅. In some embodiments, a pattern of backbone chiral centers of an oligonucleotide or a region (e.g., of a core) is or comprises Rp(Sp)₄Rp(Sp)₅. In some embodiments, a pattern of backbone chiral centers of an oligonucleotide is or comprises Np(Op)₃Rp(Sp)₄Rp(Sp)₄Rp(Op)₃Np. In some embodiments, a pattern of backbone chiral centers of an oligonucleotide is or comprises Np(Op)₃(Sp)₅Rp(Sp)₄Rp(Op)₃Np. In some embodiments, a pattern of backbone chiral centers of an oligonucleotide is or comprises Np(Op)₃(Sp)₅Rp(Sp)₅(Op)₃Np. In some embodiments, a pattern of backbone chiral centers of an oligonucleotide is or comprises Np(Op)₃Rp(Sp)₄Rp(Sp)h(Op)₃Np. In some embodiments, a pattern of backbone chiral centers of an oligonucleotide is or comprises Sp(Op)₃Rp(Sp)₄Rp(Sp)₄Rp(Op)₃Sp. In some embodiments, a pattern of backbone chiral centers of an oligonucleotide is or comprises Sp(Op)₃(Sp)₅Rp(Sp)₄Rp(Op)₃Sp. In some embodiments, a pattern of backbone chiral centers of an oligonucleotide is or comprises Sp(Op)₃(Sp)₅Rp(Sp)₅(Op)₃Sp. In some embodiments, a pattern of backbone chiral centers of an oligonucleotide is or comprises Sp(Op)₃Rp(Sp)₄Rp(Sp)₅(Op)₃Sp. In some embodiments, a pattern of backbone chiral centers of an oligonucleotide is or comprises Rp(Op)₃Rp(Sp)₄Rp(Sp)₄Rp(Op)₃Rp. In some embodiments, a pattern of backbone chiral centers of an oligonucleotide is or comprises Rp(Op)₃(Sp)₅Rp(Sp)₄Rp(Op)₃Rp. In some embodiments, a pattern of backbone chiral centers of an oligonucleotide is or comprises Rp(Op)₃(Sp)₅Rp(Sp)₅(Op)₃Rp. In some embodiments, a pattern of backbone chiral centers of an oligonucleotide is or comprises Rp(Op)₃Rp(Sp)₄Rp(Sp)₅(Op)₃Rp.

In some embodiments, each of m, y, t, n, k, f, g, h, and j is independently 1-25. In some embodiments, m is 1-25. In some embodiments, m is 1-20. In some embodiments, m is 1-15. In some embodiments, m is 1-10. In some embodiments, m is 1-5. In some embodiments, m is 2-20. In some embodiments, m is 2-15. In some embodiments, m is 2-10. In some embodiments, m is 2-5. In some embodiments, m is 1, 2, 3, 4, 5, 6, 7, 8, 9, 10, 11, 12, 13, 14, 15, 16, 17, 18, 19, 20, 21, 22, 23, 24, or 25. In some embodiments, in a pattern of backbone chiral centers each m is independently 2 or more. In some embodiments, each m is independently 2, 3, 4, 5, 6, 7, 8, 9, or 10. In some embodiments, each m is independently 2-3, 2-5, 2-6, or 2-10. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, where there are two or more occurrences of m, they can be the same or different, and each of them is independently as described in the present disclosure.

In some embodiments, y is 1-25. In some embodiments, y is 1-20. In some embodiments, y is 1-15. In some embodiments, y is 1-10. In some embodiments, y is 1-5. In some embodiments, y is 2-20. In some embodiments, y is 2-15. In some embodiments, y is 2-10. In some embodiments, y is 2-5. In some embodiments, y is 1, 2, 3, 4, 5, 6, 7, 8, 9, 10, 11, 12, 13, 14, 15, 16, 17, 18, 19, 20, 21, 22, 23, 24, or 25. In some embodiments, y is 1, 2, 3, 4, 5, 6, 7, 8, 9, or 10. In some embodiments, y is 1. In some embodiments, y is 2. In some embodiments, y is 3. In some embodiments, y is 4. In some embodiments, y is 5. In some embodiments, y is 6. In some embodiments, y is 7. In some embodiments, y is 8. In some embodiments, y is 9. In some embodiments, y is 10.

In some embodiments, t is 1-25. In some embodiments, t is 1-20. In some embodiments, t is 1-15. In some embodiments, t is 1-10. In some embodiments, t is 1-5. In some embodiments, t is 2-20. In some embodiments, t is 2-15. In some embodiments, t is 2-10. In some embodiments, t is 2-5. In some embodiments, t is 1, 2, 3, 4, 5, 6, 7, 8, 9, 10, 11, 12, 13, 14, 15, 16, 17, 18, 19, 20, 21, 22, 23, 24, or 25. In some embodiments, each t is independently 1, 2, 3, 4, 5, 6, 7, 8, 9, or 10. In some embodiments, t is 2 or more. In some embodiments, t is 1. In some embodiments, t is 2. In some embodiments, t is 3. In some embodiments, t is 4. In some embodiments, t is 5. In some embodiments, t is 6. In some embodiments, t is 7. In some embodiments, t is 8. In some embodiments, t is 9. In some embodiments, t is 10. In some embodiments, where there are two or more occurrences of t, they can be the same or different, and each of them is independently as described in the present disclosure.

In some embodiments, n is 1-25. In some embodiments, n is 1, 2, 3, 4, 5, 6, 7, 8, 9, 10, 11, 12, 13, 14, 15, 16, 17, 18, 19, 20, 21, 22, 23, 24, or 25. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10. In some embodiments, where there are two or more occurrences of n, they can be the same or different, and each of them is independently as described in the present disclosure. In many embodiments, in a pattern of backbone chiral centers, at least one occurrence of n is 1; in some cases, each n is 1.

In some embodiments, k is 1-25. In some embodiments, k is 1, 2, 3, 4, 5, 6, 7, 8, 9, 10, 11, 12, 13, 14, 15, 16, 17, 18, 19, 20, 21, 22, 23, 24, or 25. In some embodiments, k is 1. In some embodiments, k is 2. In some embodiments, k is 3. In some embodiments, k is 4. In some embodiments, k is 5. In some embodiments, k is 6. In some embodiments, k is 7. In some embodiments, k is 8. In some embodiments, k is 9. In some embodiments, k is 10. In some embodiments, f is 1-25. In some embodiments, f is 1-20. In some embodiments, f is 1-10. In some embodiments, f is 1-5. In some embodiments, f is 1, 2, 3, 4, 5, 6, 7, 8, 9, 10, 11, 12, 13, 14, 15, 16, 17, 18, 19, 20, 21, 22, 23, 24, or 25. In some embodiments, f is 1. In some embodiments, f is 2. In some embodiments, f is 3. In some embodiments, f is 4. In some embodiments, f is 5. In some embodiments, f is 6. In some embodiments, f is 7. In some embodiments, f is 8. In some embodiments, f is 9. In some embodiments, f is 10.

In some embodiments, g is 1-25. In some embodiments, g is 1-20. In some embodiments, g is 1-10. In some embodiments, g is 1-5. In some embodiments, g is 2-10. In some embodiments, g is 2-5. In some embodiments, g is 1, 2, 3, 4, 5, 6, 7, 8, 9, 10, 11, 12, 13, 14, 15, 16, 17, 18, 19, 20, 21, 22, 23, 24, or 25. In some embodiments, g is 1. In some embodiments, g is 2. In some embodiments, g is 3. In some embodiments, g is 4. In some embodiments, g is 5. In some embodiments, g is 6. In some embodiments, g is 7. In some embodiments, g is 8. In some embodiments, g is 9. In some embodiments, g is 10. In some embodiments, h is 1-25. In some embodiments, h is 1-10. In some 2% embodiments, h is 1-5. In some embodiments, h is 2-10. In some embodiments, h is 2-5. In some embodiments, h is 1, 2, 3, 4, 5, 6, 7, 8, 9, 10, 11, 12, 13, 14, 15, 16, 17, 18, 19, 20, 21, 22, 23, 24, or 25. In some embodiments, h is 1. In some embodiments, h is 2. In some embodiments, h is 3. In some embodiments, h is 4. In some embodiments, h is 5. In some embodiments, h is 6. In some embodiments, h is 7. In some embodiments, h is 8. In some embodiments, h is 9. In some embodiments, h is 10.

In some embodiments, j is 1-25. In some embodiments, j is 1-10. In some embodiments, j is 1-5. In some embodiments, j is 1, 2, 3, 4, 5, 6, 7, 8, 9, 10, 11, 12, 13, 14, 15, 16, 17, 18, 19, 20, 21, 22, 23, 24, or 25. In some embodiments, j is 1. In some embodiments, j is 2. In some embodiments, j is 3. In some embodiments, j is 4. In some embodiments, j is 5. In some embodiments, j is 6. In some embodiments, j is 7. In some embodiments, j is 8. In some embodiments, j is 9. In some embodiments, j is 10.

In some embodiments, at least one n is 1, and at least one m is no less than 2. In some embodiments, at least one n is 1, at least one t is no less than 2, and at least one m is no less than 3. In some embodiments, each n is 1. In some embodiments, t is 1. In some embodiments, at least one t>1. In some embodiments, at least one t>2. In some embodiments, at least one t>3. In some embodiments, at least one t>4. In some embodiments, at least one m>1. In some embodiments, at least one m>2. In some embodiments, at least one m>3. In some embodiments, at least one m>4. In some embodiments, a pattern of backbone chiral centers comprises one or more achiral natural phosphate linkages. In some embodiments, the sum of m, t, and n (or m and n if no t in a pattern) is no less than 5, 6, 7, 8, 9, 10, 11, 12, 13, 14, 15, 16, 17, 18, 19 or 20. In some embodiments, the sum is 5. In some embodiments, the sum is 6. In some embodiments, the sum is 7. In some embodiments, the sum is 8. In some embodiments, the sum is 9. In some embodiments, the sum is 10. In some embodiments, the sum is 11. In some embodiments, the sum is 12. In some embodiments, the sum is 13. In some embodiments, the sum is 14. In some embodiments, the sum is 15.

In some embodiments, a number of linkage phosphorus in chirally controlled internucleotidic linkages are Sp. In some embodiments, at least 10%, 20%, 25%, 30%, 35%, 40%, 45%, 50%, 55%, 60%, 65%, 70%, 75%, 80%, 85%, 90% or 95% of chirally controlled internucleotidic linkages have Sp linkage phosphorus. In some embodiments, at least 10%, 20%, 25%, 30%, 35%, 40%, 45%, 50%, 55%, 60%, 65%, 70%, 75%, 80%, 85%, 90% or 95% of all chiral internucleotidic linkages are chirally controlled internucleotidic linkages having Sp linkage phosphorus. In some embodiments, at least 10%, 20%, 25%, 30%, 35%, 40%, 45%, 50%, 55%, 60%, 65%, 70%, 75%, 80%, 85%, 90% or 95% of all internucleotidic linkages are chirally controlled internucleotidic linkages having Sp linkage phosphorus. In some embodiments, the percentage is at least 20%. In some embodiments, the percentage is at least 30%. In some embodiments, the percentage is at least 40%. In some embodiments, the percentage is at least 50%. In some embodiments, the percentage is at least 60%. In some embodiments, the percentage is at least 65%. In some embodiments, the percentage is at least 70%. In some embodiments, the percentage is at least 75%. In some embodiments, the percentage is at least 80%. In some embodiments, the percentage is at least 90%. In some embodiments, the percentage is at least 95%. In some embodiments, at least 1, 2, 3, 4, 5, 6, 7, 8, 9, 10, 11, 12, 13, 14, 15, 16, 17, 18, 19, 20, 21, 22, 23, 24, or 25 internucleotidic linkages are chirally controlled internucleotidic linkages having Sp linkage phosphorus. In some embodiments, at least 5 internucleotidic linkages are chirally controlled internucleotidic linkages having Sp linkage phosphorus. In some embodiments, at least 6 internucleotidic linkages are chirally controlled internucleotidic linkages having Sp linkage phosphorus. In some embodiments, at least 7 internucleotidic linkages are chirally controlled internucleotidic linkages having Sp linkage phosphorus. In some embodiments, at least 8 internucleotidic linkages are chirally controlled internucleotidic linkages having Sp linkage phosphorus. In some embodiments, at least 9 internucleotidic linkages are chirally controlled internucleotidic linkages having Sp linkage phosphorus. In some embodiments, at least 10 internucleotidic linkages are chirally controlled internucleotidic linkages having Sp linkage phosphorus. In some embodiments, at least 11 internucleotidic linkages are chirally controlled internucleotidic linkages having Sp linkage phosphorus. In some embodiments, at least 12 internucleotidic linkages are chirally controlled internucleotidic linkages having Sp linkage phosphorus. In some embodiments, at least 13 internucleotidic linkages are chirally controlled internucleotidic linkages having Sp linkage phosphorus. In some embodiments, at least 14 internucleotidic linkages are chirally controlled internucleotidic linkages having Sp linkage phosphorus. In some embodiments, at least 15 internucleotidic linkages are chirally controlled internucleotidic linkages having Sp linkage phosphorus. In some embodiments, at least 1, 2, 3, 4, 5, 6, 7, 8, 9, 10, 11, 12, 13, 14, 15, 16, 17, 18, 19, 20, 21, 22, 23, 24, or 25 internucleotidic linkages are chirally controlled internucleotidic linkages having Rp linkage phosphorus. In some embodiments, no more than 1, 2, 3, 4, 5, 6, 7, 8, 9, 10, 11, 12, 13, 14, 15, 16, 17, 18, 19, 20, 21, 22, 23, 24, or 25 internucleotidic linkages are chirally controlled internucleotidic linkages having Rp linkage phosphorus. In some embodiments, one and no more than one internucleotidic linkage in an oligonucleotide is a chirally controlled internucleotidic linkage having Rp linkage phosphorus. In some embodiments, 2 and no more than 2 internucleotidic linkages in an oligonucleotide are chirally controlled internucleotidic linkages having Rp linkage phosphorus. In some embodiments, 3 and no more than 3 internucleotidic linkages in an oligonucleotide are chirally controlled internucleotidic linkages having Rp linkage phosphorus. In some embodiments, 4 and no more than 4 internucleotidic linkages in an oligonucleotide are chirally controlled internucleotidic linkages having Rp linkage phosphorus. In some embodiments, 5 and no more than 5 internucleotidic linkages in an oligonucleotide are chirally controlled internucleotidic linkages having Rp linkage phosphorus.

In some embodiments, all, essentially all or most of the internucleotidic linkages in an oligonucleotide are in the Sp configuration (e.g., about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more of all chirally controlled internucleotidic linkages, or of all chiral internucleotidic linkages, or of all internucleotidic linkages in the oligonucleotide) except for one or a minority of internucleotidic linkages (e.g., 1, 2, 3, 4, or 5, and/or less than 50%, 45%, 40%, 35%, 30%, 25%, 20%, 15%, 10%, or 5% of all chirally controlled internucleotidic linkages, or of all chiral internucleotidic linkages, or of all internucleotidic linkages in the oligonucleotide) being in the Rp configuration. In some embodiments, all, essentially all or most of the internucleotidic linkages in a core are in the Sp configuration (e.g., about 50%-100%, 55%/o-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more of all chirally controlled internucleotidic linkages, or of all chiral internucleotidic linkages, or of all internucleotidic linkages, in the core) except for one or a minority of internucleotidic linkages (e.g., 1, 2, 3, 4, or 5, and/or less than 50%, 45%, 40%, 35%, 30%, 25%, 20%, 15%, 10%, or 5% of all chirally controlled internucleotidic linkages, or of all chiral internucleotidic linkages, or of all internucleotidic linkages, in the core) being in the Rp configuration. In some embodiments, all, essentially all or most of the internucleotidic linkages in the core are a phosphorothioate in the Sp configuration (e.g., about 50%-100%, 55%-100%, 60%-100%, 65%-100%, 70%-100%, 75%-100%, 80%-100%, 85%-100%, 90%-100%, 55%-95%, 60%-95%, 65%-95%, or about 55%, 60%, 65%, 70%, 75%, 80%, 85%, 90%, 95%, 97%, 99% or more of all chirally controlled internucleotidic linkages, or of all chiral internucleotidic linkages, or of all internucleotidic linkages, in the core) except for one or a minority of internucleotidic linkages (e.g., 1, 2, 3, 4, or 5, and/or less than 50%, 45%, 40%, 35%, 30%, 25%, 20%, 15%, 10%, or 5% of all chirally controlled internucleotidic linkages, or of all chiral internucleotidic linkages, or of all internucleotidic linkages, in the core) being a phosphorothioate in the Rp configuration. In some embodiments, each internucleotidic linkage in the core is a phosphorothioate in the Sp configuration except for one phosphorothioate in the Rp configuration. In some embodiments, each internucleotidic linkage in the core is a phosphorothioate in the Sp configuration except for one phosphorothioate in the Rp configuration.

In some embodiments, an oligonucleotide comprises one or more Rp internucleotidic linkages. In some embodiments, an oligonucleotide comprises one and no more than one Rp internucleotidic linkages. In some embodiments, an oligonucleotide comprises two or more Rp internucleotidic linkages. In some embodiments, an oligonucleotide comprises three or more Rp internucleotidic linkages. In some embodiments, an oligonucleotide comprises four or more Rp internucleotidic linkages. In some embodiments, an oligonucleotide comprises five or more Rp internucleotidic linkages. In some embodiments, about 5%-50% of all chirally controlled internucleotidic linkages in an oligonucleotide are Rp. In some embodiments, about 5%-40% of all chirally controlled internucleotidic linkages in an oligonucleotide are Rp. In some embodiments, about 10%-40% of all chirally controlled internucleotidic linkages in an oligonucleotide are Rp. In some embodiments, about 15%-40% of all chirally controlled internucleotidic linkages in an oligonucleotide are Rp. In some embodiments, about 20%-40% of all chirally controlled internucleotidic linkages in an oligonucleotide are Rp. In some embodiments, about 25%-40% of all chirally controlled internucleotidic linkages in an oligonucleotide are Rp. In some embodiments, about 30%-40% of all chirally controlled internucleotidic linkages in an oligonucleotide are Rp. In some embodiments, about 35%-40% of all chirally controlled internucleotidic linkages in an oligonucleotide are Rp.

In some embodiments, instead of an Rp internucleotidic linkage, a natural phosphate linkage may be similarly utilized, optionally with a modification, e.g., a sugar modification (e.g., a 5′-modification such as R^{5s} as described herein). In some embodiments, a modification improves stability of a natural phosphate linkage.

In some embodiments, the present disclosure provides an oligonucleotide having a pattern of backbone chiral centers as described herein. In some embodiments, oligonucleotides in a chirally controlled oligonucleotide composition share a common pattern of backbone chiral centers as described herein.

In some embodiments, at least about 25% of the internucleotidic linkages of a ds oligonucleotide targeting HSD17B13 are chirally controlled and have Sp linkage phosphorus. In some embodiments, at least about 30% of the internucleotidic linkages of an oligonucleotide are chirally controlled and have Sp linkage phosphorus. In some embodiments, at least about 40% of the internucleotidic linkages of a provided oligonucleotide are chirally controlled and have Sp linkage phosphorus. In some embodiments, at least about 50% of the internucleotidic linkages of a provided oligonucleotide are chirally controlled and have Sp linkage phosphorus. In some embodiments, at least about 60% of the internucleotidic linkages of a provided oligonucleotide are chirally controlled and have Sp linkage phosphorus. In some embodiments, at least about 65% of the internucleotidic linkages of a provided oligonucleotide are chirally controlled and have Sp linkage phosphorus. In some embodiments, at least about 70% of the internucleotidic linkages of a provided oligonucleotide are chirally controlled and have Sp linkage phosphorus. In some embodiments, at least about 75% of the internucleotidic linkages of a provided oligonucleotide are chirally controlled and have Sp linkage phosphorus. In some embodiments, at least about 80% of the internucleotidic linkages of a provided oligonucleotide are chirally controlled and have Sp linkage phosphorus. In some embodiments, at least about 85% of the internucleotidic linkages of a provided oligonucleotide are chirally controlled and have Sp linkage phosphorus. In some embodiments, at least about 90% of the internucleotidic linkages of a provided oligonucleotide are chirally controlled and have Sp linkage phosphorus. In some embodiments, at least about 95% of the internucleotidic linkages of a provided oligonucleotide are chirally controlled and have Sp linkage phosphorus.

In some embodiments, the present disclosure provides chirally controlled oligonucleotide compositions, e.g., chirally controlled ds oligonucleotide targeting HSD17B13 compositions, wherein the composition comprises a non-random or controlled level of a plurality of oligonucleotides, wherein oligonucleotides of the plurality share a common base sequence, and share the same configuration of linkage phosphorus independently at 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, 21, 22, 23, 24, or 25 or more chiral internucleotidic linkages.

In some embodiments, ds oligonucleotides targeting HSD17B13 comprise 2-30 chirally controlled internucleotidic linkages. In some embodiments, provided oligonucleotide compositions comprise 5-30 chirally controlled internucleotidic linkages. In some embodiments, provided oligonucleotide compositions comprise 10-30 chirally controlled internucleotidic linkages.

In some embodiments, a percentage is about 5%-100%. In some embodiments, a percentage is at least 50%, 55%, 60%, 65%, 70%, 75%, 80%, 85%, 90%, 95%, 965, 96%, 98%, or 99%. In some embodiments, a percentage is about 5%, 10%, 15%, 20%, 25%, 30%, 35%, 40%, 45%, 50%, 55%, 60%, 65%, 70%, 75%, 80%, 85%, 90%, 95%, 965, 96%, 98%, or 99%. In some embodiments, a pattern of backbone chiral centers in a ds oligonucleotide targeting HSD17B13 comprises a pattern of i^{o}-i^{s}-i^{o}-i^{s}-i⁰, i^{o}-i^{s}-i^{s}-i^{s}-i^{o}, i^{o}-i^{s}-i^{s}-i^{s}-i^{o}-i^{s}, i^{s}- i^{o}-i^{s}-i^{o}, i^{s}-i^{o}-i^{s}-i^{o}, i^{s}-i^{o}-i^{s}-i^{o}-i^{s}, i^{s}-i^{o}-i^{s}-i^{o}-i^{s}-i^{o}, i^{s}-i^{o}-i^{s}-i^{o}-i^{s}-i^{o}-i^{s}-i^{o}, i^{s}-i^{o}-i^{s}-i^{s}-i^{s}-i^{o}, i^{s}-i^{s}-i^{o}-i^{s}-i^{s}-i^{s}-i^{o}-i^{s}-i^{s}, i^{s}-i^{s}-i^{s}-i^{o}-i^{s}-i^{o}-i^{s}-i^{s}-i^{s}, i^{s}-i^{s}-i^{s}-i^{s}-i^{o}-i^{s}-i^{o}-i^{s}-i^{s}-i^{s}-i^{s}, i^{s}-i^{s}-i^{s}-i^{s}-i^{s}, i^{s}-i^{s}-i^{s}-i^{s}-i^{s}-i^{s}, i^{s}-i^{s}-i^{s}-i^{s}-i^{s}-i^{s}-i^{s}, i^{s}-i^{s}-i^{s}-i^{s}-i^{s}-i^{s}-i^{s}-i^{s}-i^{s}, i^{s}-i^{s}-i^{s}-i^{s}-i^{s}-i^{s}-i^{s}-i^{s}, or i^{r}-i^{r}-i^{r}, wherein i^{s} represents an internucleotidic linkage in the Sp configuration; i^{o} represents an achiral internucleotidic linkage; and i^{r} represents an internucleotidic linkage in the Rp configuration.

In some embodiments, an internucleotidic linkage in the Sp configuration (having a Sp linkage phosphorus) is a phosphorothioate internucleotidic linkage. In some embodiments, an achiral internucleotidic linkage is a natural phosphate linkage. In some embodiments, an internucleotidic linkage in the Rp configuration (having a Rp linkage phosphorus) is a phosphorothioate internucleotidic linkage. In some embodiments, each internucleotidic linkage in the Sp configuration is a phosphorothioate internucleotidic linkage. In some embodiments, each achiral internucleotidic linkage is a natural phosphate linkage. In some embodiments, each internucleotidic linkage in the Rp configuration is a phosphorothioate internucleotidic linkage. In some embodiments, each internucleotidic linkage in the Sp configuration is a phosphorothioate internucleotidic linkage, each achiral internucleotidic linkage is a natural phosphate linkage, and each internucleotidic linkage in the Rp configuration is a phosphorothioate internucleotidic linkage.

In some embodiments, a pattern of backbone chiral centers (e.g., a pattern of backbone chiral centers in an oligonucleotide, e.g., a ds oligonucleotide targeting HSD17B13 or in a core or a wing or in two wings of an oligonucleotide, e.g., a ds oligonucleotide targeting HSD17B13) comprises a pattern of OpSpOpSpOp, OpSpSpSpOp, OpSpSpSpOpSp, SpOpSpOp, SpOpSpOp, SpOpSpOpSp, SpOpSpOpSpOp, SpOpSpOpSpOpSpOp, SpOpSpSPSpOp, SpSpOpSpSpSpOpSpSp, SpSpSpOpSpOpSpSpSp, SpSpSpSpOpSpOpSpS SpSpSpSp, SpSpSpSpSpSp, SpSpSpSppSp, SpSpSpSpSpSpSpSp, SpSpSpSpSpSpSpSpSp, or RpRpRp, wherein each Rp and Sp is independently the linkage phosphorus configuration of a chirally controlled internucleotidic linkage (in some embodiments, each Rp and Sp is independently the linkage phosphorus configuration of a chirally controlled phosphorothioate internucleotidic linkage), and each Op independently represents linkage phosphorus being achiral in a natural phosphate linkage.

In some embodiments, a pattern of backbone chiral centers (e.g., of an oligonucleotide, e.g., a ds oligonucleotide targeting HSD17B13, or a portion thereof) is or comprises a pattern of backbone chiral centers described in Table 1.

In some embodiments, an internucleotidic linkage bonded to a wing nucleoside and a core nucleoside is considered one of the core internucleotidic linkages, for example, when describing types, modifications, numbers, and/or patterns of core internucleotidic linkages. In some embodiments, each internucleotidic linkage bonded to a wing nucleoside and a core nucleoside is considered one of the core internucleotidic linkages, for example, when describing types, modifications, numbers, and/or patterns of core internucleotidic linkages. In some embodiments, a core internucleotidic linkage is bonded to two core nucleosides. In some embodiments, a core internucleotidic linkage is bonded to a core nucleoside and a wing nucleoside. In some embodiments, each core internucleotidic linkage is independently bonded to two core nucleosides, or a core nucleoside and a wing nucleoside. In some embodiments, each wing internucleotidic linkage is independently bonded to two wing nucleosides.

In some embodiments, ds oligonucleotides targeting HSD17B13 in chirally controlled oligonucleotide compositions each comprise different types of internucleotidic linkages. In some embodiments, ds oligonucleotides targeting HSD17B13 comprise at least one natural phosphate linkage and at least one modified internucleotidic linkage. In some embodiments, ds oligonucleotides targeting HSD17B13 comprise at least one natural phosphate linkage and at least two modified internucleotidic linkages. In some embodiments, ds oligonucleotides targeting HSD17B13 comprise at least one natural phosphate linkage and at least three modified internucleotidic linkages. In some embodiments, ds oligonucleotides targeting HSD17B13 comprise at least one natural phosphate linkage and at least four modified internucleotidic linkages. In some embodiments, ds oligonucleotides targeting HSD17B13 comprise at least one natural phosphate linkage and at least five modified internucleotidic linkages. In some embodiments, ds oligonucleotides targeting HSD17B13 comprise at least one natural phosphate linkage and 1, 2, 3, 4, 5, 6, 7, 8, 9, 10, 11, 12, 13, 14, 15, 16, 17, 18, 19, 20, 21, 22, 23, 24, or 25 modified internucleotidic linkages. In some embodiments, a modified internucleotidic linkage is a phosphorothioate internucleotidic linkage. In some embodiments, each modified internucleotidic linkage is a phosphorothioate internucleotidic linkage. In some embodiments, a modified internucleotidic linkage is a phosphorothioate triester internucleotidic linkage. In some embodiments, each modified internucleotidic linkage is a phosphorothioate triester internucleotidic linkage. In some embodiments, ds oligonucleotides targeting HSD17B13 comprise at least one natural phosphate linkage and at least 2, 3, 4, 5, 6, 7, 8, 9, 10, 11, 12, 13, 14, 15, 16, 17, 18, 19, 20, 21, 22, 23, 24, or 25 consecutive modified internucleotidic linkages. In some embodiments, ds oligonucleotides targeting HSD17B13 comprise at least one natural phosphate linkage and at least 2, 3, 4, 5, 6, 7, 8, 9, 10, 11, 12, 13, 14, 15, 16, 17, 18, 19, 20, 21, 22, 23, 24, or 25 consecutive phosphorothioate internucleotidic linkages. In some embodiments, ds oligonucleotides targeting HSD17B13 comprise at least one natural phosphate linkage and at least 2, 3, 4, 5, 6, 7, 8, 9, 10, 11, 12, 13, 14, 15, 16, 17, 18, 19, 20, 21, 22, 23, 24, or 25 consecutive phosphorothioate triester internucleotidic linkages.

In some embodiments, oligonucleotides in a chirally controlled oligonucleotide composition each comprise at least two internucleotidic linkages that have different stereochemistry and/or different P-modifications relative to one another. In some embodiments, at least two internucleotidic linkages have different stereochemistry relative to one another, and the oligonucleotides each comprise a pattern of backbone chiral centers comprising alternating linkage phosphorus stereochemistry.

In some embodiments, a linkage comprises a chiral auxiliary, which, for example, is used to control the stereoselectivity of a reaction, e.g., a coupling reaction in an oligonucleotide synthesis cycle. In some embodiments, a phosphorothioate triester linkage does not comprise a chiral auxiliary. In some embodiments, a phosphorothioate triester linkage is intentionally maintained until and/or during the administration of the oligonucleotide composition to a subject.

In some embodiments, purity, particularly stereochemical purity, and particularly diastereomeric purity of many oligonucleotides and compositions thereof wherein all other chiral centers in the oligonucleotides but the chiral linkage phosphorus centers have been stereodefined (e.g., carbon chiral centers in the sugars, which are defined in, e.g., phosphoramidites for oligonucleotide synthesis), can be controlled by stereoselectivity (as appreciated by those skilled in this art, diastereoselectivity in many cases of oligonucleotide synthesis wherein the oligonucleotide comprise more than one chiral centers) at chiral linkage phosphorus in coupling steps when forming chiral internucleotidic linkages. In some embodiments, a coupling step has a stereoselectivity (diastereoselectivity when there are other chiral centers) of 60% at the linkage phosphorus. After such a coupling step, the new internucleotidic linkage formed may be referred to have a 60% stereochemical purity (for oligonucleotides, typically diastereomeric purity in view of the existence of other chiral centers). In some embodiments, each coupling step independently has a stereoselectivity of at least 60%. In some embodiments, each coupling step independently has a stereoselectivity of at least 70%. In some embodiments, each coupling step independently has a stereoselectivity of at least 80%. In some embodiments, each coupling step independently has a stereoselectivity of at least 85%. In some embodiments, each coupling step independently has a stereoselectivity of at least 90%. In some embodiments, each coupling step independently has a stereoselectivity of at least 91%. In some embodiments, each coupling step independently has a stereoselectivity of at least 92%. In some embodiments, each coupling step independently has a stereoselectivity of at least 93%. In some embodiments, each coupling step independently has a stereoselectivity of at least 94%. In some embodiments, each coupling step independently has a stereoselectivity of at least 95%. In some embodiments, each coupling step independently has a stereoselectivity of at least 96%. In some embodiments, each coupling step independently has a stereoselectivity of at least 97%. In some embodiments, each coupling step independently has a stereoselectivity of at least 98%. In some embodiments, each coupling step independently has a stereoselectivity of at least 99%. In some embodiments, each coupling step independently has a stereoselectivity of at least 99.5%. In some embodiments, each coupling step independently has a stereoselectivity of virtually 100%. In some embodiments, a coupling step has a stereoselectivity of virtually 100% in that each detectable product from the coupling step analyzed by an analytical method (e.g., NMR, HPLC, etc.) has the intended stereoselectivity. In some embodiments, a chirally controlled internucleotidic linkage is typically formed with a stereoselectivity of at least 90%, 91%, 92%, 93%, 94%, 95%, 96%, 97%, 98%, 99.5% or virtually 100% (in some embodiments, at least 90%; in some embodiments, at least 95%; in some embodiments, at least 96%; in some embodiments, at least 97%; in some embodiments, at least 98%; in some embodiments, at least 99%). In some embodiments, a chirally controlled internucleotidic linkage has a stereochemical purity (typically diastereomeric purity for oligonucleotides with multiple chiral centers) of at least 90%, 91%, 92%, 93%, 94%, 95%, 96%, 97%, 98%, 99.5% or virtually 100% (in some embodiments, at least 90%; in some embodiments, at least 95%; in some embodiments, at least 96%; in some embodiments, at least 97%; in some embodiments, at least 98%; in some embodiments, at least 99%) at its chiral linkage phosphorus. In some embodiments, each chirally controlled internucleotidic linkage independently has a stereochemical purity (typically diastereomeric purity for oligonucleotides with multiple chiral centers) of at least 90%, 91%, 92%, 93%, 94%, 95%, 96%, 97%, 98%, 99.5% or virtually 100% (in some embodiments, at least 90%; in some embodiments, at least 95%; in some embodiments, at least 96%; in some embodiments, at least 97%; in some embodiments, at least 98%; in some embodiments, at least 99%) at its chiral linkage phosphorus. In some embodiments, a non-chirally controlled internucleotidic linkage is typically formed with a stereoselectivity of less than 60%, 70%, 80%, 85%, or 90% (in some embodiments, less than 60%; in some embodiments, less than 70%; in some embodiments, less than 80%; in some embodiments, less than 85%; in some embodiments, less than 90%). In some embodiments, each non-chirally controlled internucleotidic linkage is independently formed with a stereoselectivity of less than 60%, 70%, 80%, 85%, or 90% (in some embodiments, less than 60%; in some embodiments, less than 70%; in some embodiments, less than 80%; in some embodiments, less than 85%; in some embodiments, less than 90%). In some embodiments, a non-chirally controlled internucleotidic linkage has a stereochemical purity (typically diastereomeric purity for oligonucleotides with multiple chiral centers) of less than 60%, 70%, 80%, 85%, or 90% (in some embodiments, less than 60%; in some embodiments, less than 70%; in some embodiments, less than 80%; in some embodiments, less than 85%; in some embodiments, less than 90%) at its chiral linkage phosphorus. In some embodiments, each non-chirally controlled internucleotidic linkage independently has a stereochemical purity (typically diastereomeric purity for oligonucleotides with multiple chiral centers) of less than 60%, 70%, 80%, 85%, or 90% (in some embodiments, less than 60%; in some embodiments, less than 70%; in some embodiments, less than 80%; in some embodiments, less than 85%; in some embodiments, less than 90%) at its chiral linkage phosphorus.

In some embodiments, at least 1, 2, 3, 4, 5, 6, 7, 8, 9, or 10 couplings of a monomer (as appreciated by those skilled in the art in many embodiments a phosphoramidite for oligonucleotide synthesis) independently have a stereoselectivity less than about 60%, 70%, 80%, 85%, or 90% [for oligonucleotide synthesis, typically diastereoselectivity with respect to formed linkage phosphorus chiral center(s)]. In some embodiments, at least one coupling has a stereoselectivity less than about 60%, 70%, 80%, 85%, or 90%. In some embodiments, at least two couplings independently have a stereoselectivity less than about 60%, 70%, 80%, 85%, or 90%. In some embodiments, at least three couplings independently have a stereoselectivity less than about 60%, 70%, 80%, 85%, or 90%. In some embodiments, at least four couplings independently have a stereoselectivity less than about 60%, 70%, 80%, 85%, or 90%. In some embodiments, at least five couplings independently have a stereoselectivity less than about 60%, 70%, 80%, 85%, or 90%. In some embodiments, each coupling independently has a stereoselectivity less than about 60%, 70%, 80%, 85%, or 90%. In some embodiments, each non-chirally controlled internucleotidic linkage is independently formed with a stereoselectivity less than about 60%, 70%, 80%, 85%, or 90%. In some embodiments, a stereoselectivity is less than about 60%. In some embodiments, a stereoselectivity is less than about 70%. In some embodiments, a stereoselectivity is less than about 80%. In some embodiments, a stereoselectivity is less than about 90%. In some embodiments, at least 1, 2, 3, 4, 5, 6, 7, 8, 9, 10, 11, 12, 13, 14, 15, 16, 17, 18, 19, 20, 21, 22, 23, 24, or 25 couplings independently have a stereoselectivity less than about 90%. In some embodiments, at least one coupling has a stereoselectivity less than about 90%. In some embodiments, at least two couplings have a stereoselectivity less than about 90%. In some embodiments, at least three couplings have a stereoselectivity less than about 90%. In some embodiments, at least four couplings have a stereoselectivity less than about 90%. In some embodiments, at least five couplings have a stereoselectivity less than about 90%. In some embodiments, each coupling independently has a stereoselectivity less than about 90%. In some embodiments, at least 1, 2, 3, 4, 5, 6, 7, 8, 9, 10, 11, 12, 13, 14, 15, 16, 17, 18, 19, 20, 21, 22, 23, 24, or 25 couplings independently have a stereoselectivity less than about 85%. In some embodiments, each coupling independently has a stereoselectivity less than about 85%. In some embodiments, at least 1, 2, 3, 4, 5, 6, 7, 8, 9, 10, 11, 12, 13, 14, 15, 16, 17, 18, 19, 20, 21, 22, 23, 24, or 25 couplings independently have a stereoselectivity less than about 80%. In some embodiments, each coupling independently has a stereoselectivity less than about 80%. In some embodiments, at least 1, 2, 3, 4, 5, 6, 7, 8, 9, 10, 11, 12, 13, 14, 15, 16, 17, 18, 19, 20, 21, 22, 23, 24, or 25 couplings independently have a stereoselectivity less than about 70%. In some embodiments, each coupling independently has a stereoselectivity less than about 70%. In some embodiments, oligonucleotides and compositions of the present disclosure have high purity. In some embodiments, oligonucleotides and compositions of the present disclosure have high stereochemical purity. In some embodiments, a stereochemical purity, e.g., diastereomeric purity, is about 60%-100%. In some embodiments, a diastereomeric purity, is about 60%-100%. In some embodiments, the percentage is at least 60%, 65%, 70%, 75%, 80%, 85%, 90%, 91%, 92%, 93%, 93%, 95%, 96%, 97%, 98%, or 99%. In some embodiments, the percentage is at least 80%, 85%, 90%, 91%, 92%, 93%, 93%, 95%, 96%, 97%, 98%, or 99%. In some embodiments, the percentage is at least 90%, 91%, 92%, 93%, 93%, 95%, 96%, 97%, 98%, or 99%. In some embodiments, a diastereomeric purity is at least 60%. In some embodiments, a diastereomeric purity is at least 70%. In some embodiments, a diastereomeric purity is at least 80%. In some embodiments, a diastereomeric purity is at least 85%. In some embodiments, a diastereomeric purity is at least 90%. In some embodiments, a diastereomeric purity is at least 91%. In some embodiments, a diastereomeric purity is at least 92%. In some embodiments, a diastereomeric purity is at least 93%. In some embodiments, a diastereomeric purity is at least 94%. In some embodiments, a diastereomeric purity is at least 95%. In some embodiments, a diastereomeric purity is at least 96%. In some embodiments, a diastereomeric purity is at least 97%. In some embodiments, a diastereomeric purity is at least 98%. In some embodiments, a diastereomeric purity is at least 99%. In some embodiments, a diastereomeric purity is at least 99.5%.

In some embodiments, compounds of the present disclosure (e.g., oligonucleotides, chiral auxiliaries, etc.) comprise multiple chiral elements (e.g., multiple carbon and/or phosphorus (e.g., linkage phosphorus of chiral internucleotidic linkages) chiral centers). In some embodiments, at least 1, 2, 3, 4, 5, 6, 7, 8, 9 or more chiral elements of a provided compound (e.g., an oligonucleotide) each independently have a diastereomeric purity as described herein. In some embodiments, at least 1, 2, 3, 4, 5, 6, 7, 8, 9 or more chiral carbon centers of a provided compound each independently have a diastereomeric purity as described herein. In some embodiments, at least 1, 2, 3, 4, 5, 6, 7, 8, 9 or more chiral phosphorus centers of a provided compound each independently have a diastereomeric purity as described herein. In some embodiments, each chiral element independently has a diastereomeric purity as described herein. In some embodiments, each chiral center independently has a diastereomeric purity as described herein. In some embodiments, each chiral carbon center independently has a diastereomeric purity as described herein. In some embodiments, each chiral phosphorus center independently has a diastereomeric purity as described herein. In some embodiments, each chiral phosphorus center independently has a diastereomeric purity of at least 90%, 91%, 92%, 93%, 93%, 95%, 96%, 97%, 98%, or 99% or more.

As understood by a person having ordinary skill in the art, in some embodiments, diastereoselectivity of a coupling or diastereomeric purity of a chiral linkage phosphorus center can be assessed through the diastereoselectivity of a dimer formation or diastereomeric purity of a dimer prepared under the same or comparable conditions, wherein the dimer has the same 5′- and 3′-nucleosides and internucleotidic linkage.

Various technologies can be utilized for identifying or confirming stereochemistry of chiral elements (e.g., configuration of chiral linkage phosphorus) and/or patterns of backbone chiral centers, and/or for assessing stereoselectivity (e.g., diastereoselectivity of couple steps in oligonucleotide synthesis) and/or stereochemical purity (e.g., diastereomeric purity of internucleotidic linkages, compounds (e.g., oligonucleotides), etc.). Example technologies include NMR [e.g., 1D (one-dimensional) and/or 2D (two-dimensional) ¹H-³¹P HETCOR (heteronuclear correlation spectroscopy)], HPLC, RP-HPLC, mass spectrometry, LC-MS, and cleavage of internucleotidic linkages by stereospecific nucleases, etc., which may be utilized individually or in combination. Example useful nucleases include benzonase, micrococcal nuclease, and svPDE (snake venom phosphodiesterase), which are specific for certain internucleotidic linkages with Rp linkage phosphorus (e.g., a Rp phosphorothioate linkage); and nuclease P1, mung bean nuclease, and nuclease S1, which are specific for internucleotidic linkages with Sp linkage phosphorus (e.g., a Sp phosphorothioate linkage). Without wishing to be bound by any particular theory, the present disclosure notes that, in at least some cases, cleavage of oligonucleotides by a particular nuclease may be impacted by structural elements, e.g., chemical modifications (e.g., 2′-modifications of a sugars), base sequences, or stereochemical contexts. For example, it is observed that in some cases, benzonase and micrococcal nuclease, which are specific for internucleotidic linkages with Rp linkage phosphorus, were unable to cleave an isolated Rp phosphorothioate internucleotidic linkage flanked by Sp phosphorothioate internucleotidic linkages.

In some embodiments, oligonucleotides sharing a common base sequence, a common pattern of backbone linkages, and a common pattern of backbone chiral centers share a common pattern of backbone phosphorus modifications and a common pattern of base modifications. In some embodiments, oligonucleotide compositions sharing a common base sequence, a common pattern of backbone linkages, and a common pattern of backbone chiral centers share a common pattern of backbone phosphorus modifications and a common pattern of nucleoside modifications. In some embodiments, oligonucleotides share a common base sequence, a common pattern of backbone linkages, and a common pattern of backbone chiral centers have identical structures.

In some embodiments, the present disclosure provides an oligonucleotide composition comprising a plurality of oligonucleotides capable of directing HSD17B13 knockdown, wherein oligonucleotides of the plurality are of a particular oligonucleotide type, which composition is chirally controlled in that it is enriched, relative to a substantially racemic preparation of oligonucleotides having the same base sequence, for oligonucleotides of the particular oligonucleotide type.

In some embodiments, oligonucleotides having a common base sequence, a common pattern of backbone linkages, and a common pattern of backbone chiral centers have a common pattern of backbone phosphorus modifications and a common pattern of base modifications. In some embodiments, oligonucleotides having a common base sequence, a common pattern of backbone linkages, and a common pattern of backbone chiral centers have a common pattern of backbone phosphorus modifications and a common pattern of nucleoside modifications. In some embodiments, oligonucleotides having a common base sequence, a common pattern of backbone linkages, and a common pattern of backbone chiral centers have identical structures. In some embodiments, the present disclosure provides ds oligonucleotide targeting HSD17B13 compositions comprising a plurality of oligonucleotides. In some embodiments, the present disclosure provides chirally controlled oligonucleotide compositions of ds oligonucleotides targeting HSD17B13. In some embodiments, the present disclosure provides a ds oligonucleotide targeting HSD17B13 whose base sequence is or is complementary to a HSD17B13 sequence disclosed herein or a portion thereof (e.g., various bases sequences in Table 1, wherein each T may be independently replaced with U and vice versa). In some embodiments, the present disclosure provides a ds oligonucleotide targeting HSD17B13 whose base sequence comprises a base sequence that is or is complementary to a HSD17B13 sequence disclosed herein or a portion thereof (e.g., various bases sequences in Table 1). In some embodiments, the present disclosure provides a ds oligonucleotide targeting HSD17B13 whose base sequence comprises 15 contiguous bases of a base sequence that is or is complementary to a HSD17B13 sequence disclosed herein or a portion thereof (e.g., various bases sequences in Table 1, wherein each T may be independently replaced with U and vice versa). In some embodiments, the present disclosure provides a ds oligonucleotide targeting HSD17B13 which has a base sequence comprising 15 contiguous bases with 0-3 mismatches of a base sequence that is or is complementary to a HSD17B13 sequence disclosed herein or a portion thereof (e.g., various bases sequences in Table 1, wherein each T may be independently replaced with U and vice versa). In some embodiments, the present disclosure provides a ds oligonucleotide targeting HSD17B13 composition wherein the ds oligonucleotides targeting HSD17B13 comprise at least one chiral internucleotidic linkage which is not chirally controlled. In some embodiments, the present disclosure provides a ds oligonucleotide targeting HSD17B13 comprising a non-chirally controlled chiral internucleotidic linkage, wherein the base sequence of the ds oligonucleotide targeting HSD17B13 comprises a base sequence that is or is complementary to a HSD17B13 sequence disclosed herein or a portion thereof (e.g., various bases sequences in Table 1, wherein each T may be independently replaced with U and vice versa). In some embodiments, the present disclosure provides a ds oligonucleotide targeting HSD17B13 composition comprising a non-chirally controlled chiral internucleotidic linkage, wherein the base sequence of the ds oligonucleotide targeting HSD17B13 is a base sequence that is or is complementary to a HSD17B13 sequence disclosed herein or a portion thereof (e.g., various bases sequences in Table 1, wherein each T may be independently replaced with U and vice versa). In some embodiments, the present disclosure provides a ds oligonucleotide targeting HSD17B13 comprising a non-chirally controlled chiral internucleotidic linkage, wherein the base sequence of the ds oligonucleotide targeting HSD17B13 comprises 15 contiguous bases of a base sequence that is or is complementary to a HSD17B13 sequence disclosed herein or a portion thereof (e.g., various bases sequences in Table 1, wherein each T may be independently replaced with U and vice versa). In some embodiments, the present disclosure provides a ds oligonucleotide targeting HSD17B13 comprising a non-chirally controlled chiral internucleotidic linkage, wherein the base sequence of the ds oligonucleotides targeting HSD17B13 comprises 15 contiguous bases with 0-3 mismatches of a base sequence that is or is complementary to a HSD17B13 sequence disclosed herein or a portion thereof (e.g., various bases sequences in Table 1, wherein each T may be independently replaced with U and vice versa). In some embodiments, the present disclosure provides a ds oligonucleotide targeting HSD17B13 comprising a chirally controlled chiral internucleotidic linkage, wherein the base sequence of the ds oligonucleotide targeting HSD17B13 comprises a base sequence that is or is complementary to a HSD17B13 sequence disclosed herein or a portion thereof (e.g., various bases sequences in Table 1, wherein each T may be independently replaced with U and vice versa). In some embodiments, the present disclosure provides a ds oligonucleotide targeting HSD17B13 composition comprising a chirally controlled chiral internucleotidic linkage, wherein the base sequence of the ds oligonucleotide targeting HSD17B13 is a base sequence that is or is complementary to a HSD17B13 sequence disclosed herein or a portion thereof (e.g., various bases sequences in Table 1, wherein each T may be independently replaced with U and vice versa). In some embodiments, the present disclosure provides a ds oligonucleotide targeting HSD17B13 comprising a chirally controlled chiral internucleotidic linkage, wherein the base sequence of the ds oligonucleotide targeting HSD17B13 comprises 15 contiguous bases of a base sequence that is or is complementary to a HSD17B13 sequence disclosed herein or a portion thereof (e.g., various bases sequences in Table 1, wherein each T may be independently replaced with U and vice versa). In some embodiments, the present disclosure provides a ds oligonucleotide targeting HSD17B13 comprising a chirally controlled chiral internucleotidic linkage, wherein the base sequence of the ds oligonucleotides targeting HSD17B13 comprises 15 contiguous bases with 0-3 mismatches of a base sequence that is or is complementary to a HSD17B13 sequence disclosed herein or a portion thereof (e.g., various bases sequences in Table 1, wherein each T may be independently replaced with U and vice versa).

In some embodiments, oligonucleotides of the same oligonucleotide type have a common pattern of backbone phosphorus modifications and a common pattern of nucleoside modifications. In some embodiments, oligonucleotides of the same oligonucleotide type have a common pattern of sugar modifications. In some embodiments, oligonucleotides of the same oligonucleotide type have a common pattern of base modifications. In some embodiments, oligonucleotides of the same oligonucleotide type have a common pattern of nucleoside modifications. In some embodiments, oligonucleotides of the same oligonucleotide type have the same constitution. In many embodiments, oligonucleotides of the same oligonucleotide type are identical. In some embodiments, oligonucleotides of the same oligonucleotide type are of the same oligonucleotide (as those skilled in the art will appreciate, such oligonucleotides may each independently exist in one of the various forms of the oligonucleotide, and may be the same, or different forms of the oligonucleotide). In some embodiments, oligonucleotides of the same oligonucleotide type are each independently of the same oligonucleotide or a pharmaceutically acceptable salt thereof.

In some embodiments, a plurality of oligonucleotides or oligonucleotides of a particular oligonucleotide type in a provided oligonucleotide composition are ds oligonucleotides targeting HSD17B13. In some embodiments, the present disclosure provides a chirally controlled ds oligonucleotide targeting HSD17B13 composition comprising a plurality of ds oligonucleotides targeting HSD17B13, wherein the oligonucleotides share:
1) a common base sequence;
2) a common pattern of backbone linkages; and
3) the same linkage phosphorus stereochemistry at one or more chiral internucleotidic linkages (chirally controlled internucleotidic linkages),
wherein the composition is enriched, relative to a substantially racemic preparation of oligonucleotides sharing the common base sequence and pattern of backbone linkages, for oligonucleotides of the plurality.

In some embodiments, as used herein, “one or more” or “at least one” is 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more.

In some embodiments, an oligonucleotide type is further defined by: 4) additional chemical moiety, if any.

In some embodiments, the percentage is at least about 10%. In some embodiments, the percentage is at least about 20%. In some embodiments, the percentage is at least about 30%. In some embodiments, the percentage is at least about 40%. In some embodiments, the percentage is at least about 50%. In some embodiments, the percentage is at least about 60%. In some embodiments, the percentage is at least about 70%. In some embodiments, the percentage is at least about 75%. In some embodiments, the percentage is at least about 80%. In some embodiments, the percentage is at least about 85%. In some embodiments, the percentage is at least about 90%. In some embodiments, the percentage is at least about 91%. In some embodiments, the percentage is at least about 92%. In some embodiments, the percentage is at least about 93%. In some embodiments, the percentage is at least about 94%. In some embodiments, the percentage is at least about 95%. In some embodiments, the percentage is at least about 96%. In some embodiments, the percentage is at least about 97%. In some embodiments, the percentage is at least about 98%. In some embodiments, the percentage is at least about 99%. In some embodiments, the percentage is or is greater than (DS)^{nc}, wherein DS and nc are each independently as described in the present disclosure. In some embodiments, a plurality of oligonucleotides, e.g., ds oligonucleotides targeting HSD17B13, share the same constitution. In some embodiments, a plurality of oligonucleotides, e.g., ds oligonucleotides targeting HSD17B13, are identical (the same stereoisomer). In some embodiments, a chirally controlled oligonucleotide composition, e.g., a chirally controlled ds oligonucleotide targeting HSD17B13 composition, is a stereopure oligonucleotide composition wherein oligonucleotides of the plurality are identical (the same stereoisomer), and the composition does not contain any other stereoisomers. Those skilled in the art will appreciate that one or more other stereoisomers may exist as impurities as processes, selectivities, purifications, etc. may not achieve completeness.

In some embodiments, a provided composition is characterized in that when it is contacted with a target nucleic acid (e.g., a HSD17B13 transcript (e.g., pre-mRNA, mature mRNA, other types of RNA, etc. that hybridizes with oligonucleotides of the composition)), levels of the target nucleic acid and/or a product encoded thereby is reduced compared to that observed under a reference condition. In some embodiments, a reference condition is selected from the group consisting of absence of the composition, presence of a reference composition, and combinations thereof. In some embodiments, a reference condition is absence of the composition. In some embodiments, a reference condition is presence of a reference composition. In some embodiments, a reference composition is a composition whose oligonucleotides do not hybridize with the target nucleic acid. In some embodiments, a reference composition is a composition whose oligonucleotides do not comprise a sequence that is sufficiently complementary to the target nucleic acid. In some embodiments, a provided composition is a chirally controlled oligonucleotide composition and a reference composition is a non-chirally controlled oligonucleotide composition which is otherwise identical but is not chirally controlled (e.g., a racemic preparation of oligonucleotides of the same constitution as oligonucleotides of a plurality in the chirally controlled oligonucleotide composition).

In some embodiments, the present disclosure provides a chirally controlled ds oligonucleotide targeting HSD17B13 composition comprising a plurality of ds oligonucleotides targeting HSD17B13 capable of directing HSD17B13 knockdown, wherein the oligonucleotides share:
1) a common base sequence,
2) a common pattern of backbone linkages, and
3) the same linkage phosphorus stereochemistry at one or more (e.g., 1-50, 1-40, 1-30, 1-25, 1-20, 1-15, 1-10, 5-50, 5-40, 5-30, 5-25, 5-20, 5-15, 5-10, 1, 2, 3, 4, 5, 6, 7, 8, 9, 10, 11, 12, 13, 14, 15, 16, 17, 18, 19, 20, or more) chiral internucleotidic linkages (chirally controlled internucleotidic linkages),
wherein the composition is enriched, relative to a substantially racemic preparation of oligonucleotides sharing the common base sequence and pattern of backbone linkages, for oligonucleotides of the plurality,
the oligonucleotide composition being characterized in that, when it is contacted with a HSD17B13 transcript in a HSD17B13 knockdown system, knockdown of the transcript is improved relative to that observed under reference conditions selected from the group consisting of absence of the composition, presence of a reference composition, and combinations thereof.

As noted above and understood in the art, in some embodiments, the base sequence of an oligonucleotide may refer to the identity and/or modification status of nucleoside residues (e.g., of sugar and/or base components, relative to standard naturally occurring nucleotides such as adenine, cytosine, guanosine, thymine, and uracil) in the oligonucleotide and/or to the hybridization character (i.e., the ability to hybridize with particular complementary residues) of such residues.

As demonstrated herein, oligonucleotide structural elements (e.g., patterns of sugar modifications, backbone linkages, backbone chiral centers, backbone phosphorus modifications, etc.) and combinations thereof can provide surprisingly improved properties and/or bioactivities.

In some embodiments, oligonucleotide compositions are capable of reducing the expression, level and/or activity of a target gene or a gene product thereof. In some embodiments, oligonucleotide compositions are capable of reducing in the expression, level and/or activity of a target gene or a gene product thereof by sterically blocking translation after annealing to a target gene mRNA, by cleaving mRNA (pre-mRNA or mature mRNA), and/or by altering or interfering with mRNA splicing. In some embodiments, provided ds oligonucleotide targeting HSD17B13 compositions are capable of reducing the expression, level and/or activity of a HSD17B13 target gene or a gene product thereof. In some embodiments, provided ds oligonucleotide targeting HSD17B13 compositions are capable of reducing in the expression, level and/or activity of a HSD17B13 target gene or a gene product thereof by sterically blocking translation after annealing to a HSD17B13 target gene mRNA, by cleaving HSD17B13 mRNA (pre-mRNA or mature mRNA), and/or by altering or interfering with mRNA splicing.

In some embodiments, an oligonucleotide composition, e.g., a ds oligonucleotide targeting HSD17B13 composition, is a substantially pure preparation of a single oligonucleotide stereoisomer, e.g., a ds oligonucleotide targeting HSD17B13 stereoisomer, in that oligonucleotides in the composition that are not of the oligonucleotide stereoisomer are impurities from the preparation process of said oligonucleotide stereoisomer, in some case, after certain purification procedures.

In some embodiments, the present disclosure provides oligonucleotides and oligonucleotide compositions that are chirally controlled, and in some embodiments, stereopure. For instance, in some embodiments, a provided composition contains non-random or controlled levels of one or more individual oligonucleotide types as described herein. In some embodiments, oligonucleotides of the same oligonucleotide type are identical.

### Sugars

Various sugars, including modified sugars, can be utilized in accordance with the present disclosure. In some embodiments, the present disclosure provides sugar modifications and patterns thereof optionally in combination with other structural elements (e.g., internucleotidic linkage modifications and patterns thereof, pattern of backbone chiral centers thereof, etc.) that when incorporated into oligonucleotides can provide improved properties and/or activities.

The most common naturally occurring nucleosides comprise ribose sugars (e.g., in RNA) or deoxyribose sugars (e.g., in DNA) linked to the nucleobases adenosine (A), cytosine (C), guanine (G), thymine (T) or uracil (U). In some embodiments, a sugar, e.g., various sugars in many oligonucleotides in Table 1 (unless otherwise notes), is a natural DNA sugar (in DNA nucleic acids or oligonucleotides, having the structure of

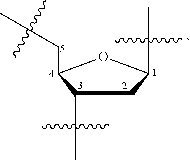 [SMILES: *C[C@H]1O[C@@H](*)C[C@H]1*]

wherein a nucleobase is attached to the 1′ position, and the 3′ and 5′ positions are connected to internucleotidic linkages (as appreciated by those skilled in the art, if at the 5′-end of an oligonucleotide, the 5′ position may be connected to a 5′-end group (e.g., —OH), and if at the 3′-end of an oligonucleotide, the 3′ position may be connected to a 3′-end group (e.g., —OH). In some embodiments, a sugar is a natural RNA sugar (in RNA nucleic acids or oligonucleotides, having the structure of

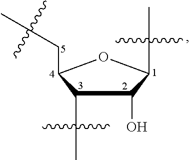

wherein a nucleobase is attached to the 1′ position, and the 3′ and 5′ positions are connected to internucleotidic linkages (as appreciated by those skilled in the art, if at the 5′-end of an oligonucleotide, the 5′ position may be connected to a 5′-end group (e.g., —OH), and if at the 3′-end of an oligonucleotide, the 3′ position may be connected to a 3′-end group (e.g., —OH). In some embodiments, a sugar is a modified sugar in that it is not a natural DNA sugar or a natural RNA sugar. Among other things, modified sugars may provide improved stability. In some embodiments, modified sugars can be utilized to alter and/or optimize one or more hybridization characteristics. In some embodiments, modified sugars can be utilized to alter and/or optimize target recognition. In some embodiments, modified sugars can be utilized to optimize Tm. In some embodiments, modified sugars can be utilized to improve oligonucleotide activities.

Sugars can be bonded to internucleotidic linkages at various positions. As non-limiting examples, internucleotidic linkages can be bonded to the 2′, 3′, 4′ or 5′ positions of sugars. In some embodiments, as most commonly in natural nucleic acids, an internucleotidic linkage connects with one sugar at the 5′ position and another sugar at the 3′ position unless otherwise indicated.

In some embodiments, a sugar is an optionally substituted natural DNA or RNA sugar. In some embodiments, a sugar is optionally substituted

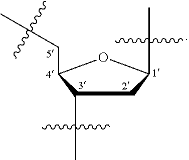 [SMILES: *C[C@H]1O[C@@H](*)C[C@H]1*]

In some embodiments, the 2′ position is optionally substituted. In some embodiments, a sugar is

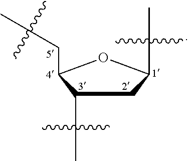 [SMILES: *C[C@H]1O[C@@H](*)C[C@H]1*]

In some embodiments, a sugar has the structure of

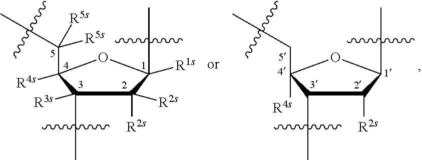

wherein each of R^{1s}, R^{2s}, R^{3s}, R^{4s}, and R^{5s} is independently —H, a suitable substituent or suitable sugar modification (e.g., those described in U.S. Pat. Nos. 9,394,333, 9,744,183, 9,605,019, 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO 2019/055951, WO 2019/075357, WO 2019/032612, and/or WO 2020/191252, the substituents, sugar modifications, descriptions of R^{1s}, R^{2s}, R^{3s}, R^{4s}, and R^{5s}, and modified sugars of each of which are independently incorporated herein by reference). In some embodiments, a sugar has the structure of

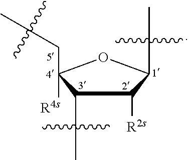

In some embodiments, R^{4s} is —H. In some embodiments, a sugar has the structure of

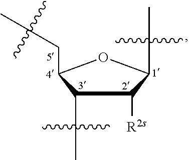

wherein R^{2s} is —H, halogen, or —OR, wherein R is optionally substituted C₁₋₆ aliphatic. In some embodiments, R^{2s} is —H. In some embodiments, R^{2s} is —F. In some embodiments, R^{2s} is —OMe. In some embodiments, R^{2s} is —OCH₂CH₂OMe.

In some embodiments, a sugar has the structure of

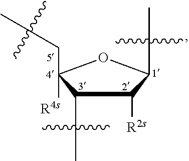

wherein R^{2s} and R^{4s} are taken together to form -L^{s}-, wherein L^{s} is a covalent bond or optionally substituted bivalent C₁₋₆ aliphatic or heteroaliphatic having 1-4 heteroatoms. In some embodiments, each heteroatom is independently selected from nitrogen, oxygen or sulfur). In some embodiments, L^{s} is optionally substituted C2-O—CH₂—C4. In some embodiments, L^{s} is C2-O—CH₂—C4. In some embodiments, L^{s} is C2-O—(R)—CH(CH₂CH₃)—C4. In some embodiments, L^{s} is C2-O—(S)—CH(CH₂CH₃)—C4.

In some embodiments, a sugar is a bicyclic sugar, e.g., sugars wherein R^{2s} and R^{4s} are taken together to form a link as described in the present disclosure. In some embodiments, a sugar is selected from LNA sugars, BNA sugars, cEt sugars, etc. In some embodiments, a bridge is between the 2′ and 4′-carbon atoms (corresponding to R^{2s} and R^{4s} taken together with their intervening atoms to form an optionally substituted ring as described herein). In some embodiments, examples of bicyclic sugars include alpha-L-methyleneoxy (4′-CH₂—O-2′) LNA, beta-D-methyleneoxy (4′-CH₂—O-2′) LNA, ethyleneoxy (4′-(CH₂)₂—O-2′) LNA, aminooxy (4′ —CH₂—O—N(R)-2′) LNA, and oxyamino (4′-CH₂—N(R)—O-2′) LNA. In some embodiments, a bicyclic sugar, e.g., a LNA or BNA sugar, is sugar having at least one bridge between two sugar carbons. In some embodiments, a bicyclic sugar in a nucleoside may have the stereochemical configurations of alpha-L-ribofuranose or beta-D-ribofuranose. In some embodiments, a sugar is a sugar described in WO 1999014226. In some embodiments, a 4′-2′ bicyclic sugar or 4′ to 2′ bicyclic sugar is a bicyclic sugar comprising a furanose ring which comprises a bridge connecting the 2′ carbon atom and the 4′ carbon atom of the sugar ring. In some embodiments, a bicyclic sugar, e.g., a LNA or BNA sugar, comprises at least one bridge between two pentofuranosyl sugar carbons. In some embodiments, a LNA or BNA sugar, comprises at least one bridge between the 4′ and the 2′ pentofuranosyl sugar carbons.

In some embodiments, a bicyclic sugar is a sugar of alpha-L-methyleneoxy (4′-CH₂—O-2′) BNA, beta-D-methyleneoxy (4′-CH₂—O-2′) BNA, ethyleneoxy (4′-(CH₂)₂—O-2′) BNA, aminooxy (4′-CH₂—O—N(R)-2′) BNA, oxyamino (4′-CH₂—N(R)—O-2′) BNA, methyl(methyleneoxy) (4′-CH(CH₃)—O-2′) BNA (also referred to as constrained ethyl or cEt), methylene-thio (4′-CH₂—S-2′) BNA, methylene-amino (4′-CH₂—N(R)-2′) BNA, methyl carbocyclic (4′-CH₂—CH(CH₃)-2′) BNA, propylene carbocyclic (4′-(CH₂)₃-2′) BNA, or vinyl BNA.

In some embodiments, a sugar modification is 2′-OMe, 2′-MOE, 2′-LNA, 2′-F, 5′-vinyl, or S-cEt. In some embodiments, a modified sugar is a sugar of FRNA, FANA, or morpholino. In some embodiments, an oligonucleotide comprises a nucleic acid analog, e.g., GNA, LNA, PNA, TNA, F-HNA (F-THP or 3′-fluoro tetrahydropyran), MNA (mannitol nucleic acid, e.g., Leumann 2002 Bioorg. Med. Chem. 10: 841-854), ANA (anitol nucleic acid), or morpholino, or a portion thereof. In some embodiments, a sugar modification replaces a natural sugar with another cyclic or acyclic moiety. Examples of such moieties are widely known in the art, e.g., those used in morpholino, glycol nucleic acids, etc. and may be utilized in accordance with the present disclosure. As appreciated by those skilled in the art, when utilized with modified sugars, in some embodiments internucleotidic linkages may be modified, e.g., as in morpholino, PNA, etc.

In some embodiments, a sugar is a 6′-modified bicyclic sugar that have either (R) or (S)-chirality at the 6-position, e.g., those described in U.S. Pat. No. 7,399,845. In some embodiments, a sugar is a 5′-modified bicyclic sugar that has either (R) or (S)-chirality at the 5-position, e.g., those described in US 20070287831.

In some embodiments, a modified sugar contains one or more substituents at the 2′ position (typically one substituent, and often at the axial position) independently selected from —F; —CF₃, —CN, —N₃, —NO, —NO₂, —OR′, —SR′, or —N(R′)₂, wherein each R′ is independently optionally substituted C₁₋₁₀ aliphatic; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein each of the alkyl, alkylene, alkenyl and alkynyl is independently and optionally substituted. In some embodiments, a substituent is —O(CH₂)ₙOCH₃, —O(CH₂)ₙNH₂, MOE, DMAOE, or DMAEOE, wherein n is from 1 to about 10. In some embodiments, a modified sugar is one described in WO 2001/088198; and Martin et al., Helv. Chim. Acta, 1995, 78, 486-504. In some embodiments, a modified sugar comprises one or more groups selected from a substituted silyl group, an RNA cleaving group, a reporter group, a fluorescent label, an intercalator, a group for improving the pharmacokinetic properties of a nucleic acid, a group for improving the pharmacodynamic properties of a nucleic acid, or other substituents having similar properties. In some embodiments, modifications are made at one or more of the 2′, 3′, 4′, or 5′ positions, including the 3′ position of the sugar on the 3′-terminal nucleoside or in the 5′ position of the 5′-terminal nucleoside.

In some embodiments, the 2′-OH of a ribose is replaced with a group selected from —H, —F; —CF₃, —CN, —N₃, —NO, —NO₂, —OR′, —SR′, or —N(R′)₂, wherein each R′ is independently described in the present disclosure; —O(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)C₁-C₁₀ alkylene)-O—(C₁-C₁₀ alkyl), wherein each of the alkyl, alkylene, alkenyl and alkynyl is independently and optionally substituted. In some embodiments, the 2′-OH is replaced with —H (deoxyribose). In some embodiments, the 2′-OH is replaced with —F. In some embodiments, the 2′-OH is replaced with —OR′. In some embodiments, the 2′-OH is replaced with —OMe. In some embodiments, the 2′-OH is replaced with —OCH₂CH₂OMe.

In some embodiments, a sugar modification is a 2′-modification. Commonly used 2′-modifications include but are not limited to 2′-OR, wherein R is optionally substituted C₁₋₆ aliphatic. In some embodiments, a modification is 2′-OR, wherein R is optionally substituted C₁₋₆ alkyl. In some embodiments, a modification is 2′-OMe. In some embodiments, a modification is 2′-MOE. In some embodiments, a 2′-modification is S-cEt. In some embodiments, a modified sugar is an LNA sugar. In some embodiments, a 2′-modification is —F. In some embodiments, a 2′-modification is FANA. In some embodiments, a 2′-modification is FRNA. In some embodiments, a sugar modification is a 5′-modification, e.g., 5′-Me. In some embodiments, a sugar modification changes the size of the sugar ring. In some embodiments, a sugar modification is the sugar moiety in FHNA.

In some embodiments, a sugar modification replaces a sugar moiety with another cyclic or acyclic moiety. Examples of such moieties are widely known in the art, including but not limited to those used in morpholino (optionally with its phosphorodiamidate linkage), glycol nucleic acids, etc.

In some embodiments, one or more of the sugars of a ds oligonucleotide targeting HSD17B13 are modified. In some embodiments, each sugar of an oligonucleotide is independently modified. In some embodiments, a modified sugar comprises a 2′-modification. In some embodiments, each modified sugar independently comprises a 2′-modification. In some embodiments, a 2′-modification is 2′-OR, wherein R is optionally substituted C₁₋₆ aliphatic. In some embodiments, a 2′-modification is a 2′-OMe. In some embodiments, a 2′-modification is a 2′-MOE. In some embodiments, a 2′-modification is an LNA sugar modification. In some embodiments, a 2′-modification is 2′-F. In some embodiments, each sugar modification is independently a 2′-modification. In some embodiments, each sugar modification is independently 2′-OR. In some embodiments, each sugar modification is independently 2′-OR, wherein R is optionally substituted C₁₋₆ alkyl. In some embodiments, each sugar modification is 2′-OMe. In some embodiments, each sugar modification is 2′-MOE. In some embodiments, each sugar modification is independently 2′-OMe or 2′-MOE. In some embodiments, each sugar modification is independently 2′-OMe, 2′-MOE, or a LNA sugar.

In some embodiments, a modified sugar is an optionally substituted ENA sugar. In some embodiments, a sugar is one described in, e.g., Seth et al., J Am Chem Soc. 2010 Oct. 27; 132(42): 14942-14950. In some embodiments, a modified sugar is a sugar in XNA (xenonucleic acid), for instance, arabinose, anhydrohexitol, threose, 2′fluoroarabinose, or cyclohexene.

Modified sugars include cyclobutyl or cyclopentyl moieties in place of a pentofuranosyl sugar. Representative examples of such modified sugars include those described in U.S. Pat. Nos. 4,981,957, 5,118,800, 5,319,080, or U.S. Pat. No. 5,359,044. In some embodiments, the oxygen atom within the ribose ring is replaced by nitrogen, sulfur, selenium, or carbon. In some embodiments, —O— is replaced with —N(R′)—, —S—, —Se— or —C(R′)₂—. In some embodiments, a modified sugar is a modified ribose wherein the oxygen atom within the ribose ring is replaced with nitrogen, and wherein the nitrogen is optionally substituted with an alkyl group (e.g., methyl, ethyl, isopropyl, etc.).

In some embodiments, sugars are connected by internucleotidic linkages, in some embodiments, modified internucleotidic linkage. In some embodiments, an internucleotidic linkage does not contain a linkage phosphorus. In some embodiments, an internucleotidic linkage is -L-. In some embodiments, an internucleotidic linkage is —OP(O)(—C≡CH)O—, —OP(O)(R)O— (e.g., R is —CH₃), 3′-NHP(O)(OH)O— 5′, 3′-OP(O)(CH₃)OCH₂— 5′, 3′-CH₂C(O)NHCH₂-5′, 3′-SCH₂OCH₂-5′, 3′-OCH₂OCH₂-5′, 3′-CH₂NR′CH₂-5′, 3′-CH₂N(Me)OCH₂-5′, 3′-NHC(O)CH₂CH₂-5′, 3′-NR′C(O)CH₂CH₂-5′, 3′-CH₂CH₂NR′-5′, 3′-CH₂CH₂NH-5′, or 3′-OCH₂CH₂N(R′)-5′. In some embodiments, a 5′ carbon may be optionally substituted with ═O.

In some embodiments, a modified sugar is an optionally substituted pentose or hexose. In some embodiments, a modified sugar is an optionally substituted pentose. In some embodiments, a modified sugar is an optionally substituted hexose. In some embodiments, a modified sugar is an optionally substituted ribose or hexitol. In some embodiments, a modified sugar is an optionally substituted ribose. In some embodiments, a modified sugar is an optionally substituted hexitol.

In some embodiments, a sugar modification is 5′-vinyl (R or S), 5′-methyl (R or S), 2′-SH, 2′-F, 2′-OCH₃, 2′-OCH₂CH₃, 2′-OCH₂CH₂F or 2′-O(CH₂)₂OCH₃. In some embodiments, a substituent at the 2′ position, e.g., a 2′-modification, is allyl, amino, azido, thio, 0-allyl, 0-C₁-C₁₀ alkyl, OCF₃, OCH₂F, O(CH₂)₂SCH₃, O(CH₂)₂—O—N(Rₘ)Rₙ), O—CH₂—C(═O)—N(Rₘ)(Rₙ), and O—CH₂—C(═O)—N(Rₗ)—(CH₂)₂—N(Rₘ)(Rₙ), wherein each allyl, amino and alkyl is optionally substituted, and each of Rₗ, Rₘ and Rₙ is independently R′ as described in the present disclosure. In some embodiments, each of Rₗ, Rₘ and Rₙ is independently —H or optionally substituted C₁-C₁₀ alkyl.

In some embodiments, a sugar is a tetrahydropyran or THP sugar. In some embodiments, a modified nucleoside is tetrahydropyran nucleoside or THP nucleoside which is a nucleoside having a six-membered tetrahydropyran sugar substituted for a pentofuranosyl residue in typical natural nucleosides. THP sugars and/or nucleosides include those used in hexitol nucleic acid (HNA), anitol nucleic acid (ANA), mannitol nucleic acid (MNA) (e.g., Leumann, Bioorg. Med. Chem., 2002, 10, 841-854) or fluoro HNA (F-HNA).

In some embodiments, sugars comprise rings having more than 5 atoms and/or more than one heteroatom, e.g., morpholino sugars.

As those skilled in the art will appreciate, modifications of sugars, nucleobases, internucleotidic linkages, etc. can and are often utilized in combination in oligonucleotides, e.g., see various oligonucleotides in Table 1. For example, a combination of sugar modification and nucleobase modification is 2′-F (sugar) 5-methyl (nucleobase) modified nucleosides. In some embodiments, a combination is replacement of a ribosyl ring oxygen atom with S and substitution at the 2′-position.

In some embodiments, a 2′-modified sugar is a furanosyl sugar modified at the 2′ position. In some embodiments, a 2′-modification is halogen, —R′ (wherein R′ is not —H), —OR′ (wherein R′ is not —H), —SR′, —N(R′)₂, optionally substituted —CH₂—CH═CH₂, optionally substituted alkenyl, or optionally substituted alkynyl. In some embodiments, a 2′-modifications is selected from —O[(CH₂)ₙO]ₘCH₃, —O(CH₂)ₙNH₂, —O(CH₂)·CH₃, —O(CH₂)ₙF, —O(CH₂)ₙONH₂, —OCH₂C(═O)N(H)CH₃, and —O(CH₂)ₙON[(CH₂)·CH₃]₂, wherein each n and m is independently from 1 to about 10. In some embodiments, a 2′-modification is optionally substituted C₁-C₁₂ alkyl, optionally substituted alkenyl, optionally substituted alkynyl, optionally substituted alkaryl, optionally substituted aralkyl, optionally substituted —O-alkaryl, optionally substituted —O-aralkyl, —SH, —SCH₃, —OCN, —Cl, —Br, —CN, —F, —CF₃, —OCF₃, —SOCH₃, —SO₂CH₃, —ONO₂, —NO₂, —N₃, —NH₂, optionally substituted heterocycloalkyl, optionally substituted heterocycloalkaryl, optionally substituted aminoalkylamino, optionally substituted polyalkylamino, substituted silyl, a reporter group, an intercalator, a group for improving pharmacokinetic properties, a group for improving the pharmacodynamic properties, and other substituents. In some embodiments, a 2′-modification is a 2′-MOE modification.

In some embodiments, a 2′-modified or 2′-substituted sugar or nucleoside is a sugar or nucleoside comprising a substituent at the 2′ position of the sugar which is other than —H (typically not considered a substituent) or —OH. In some embodiments, a 2′-modified sugar is a bicyclic sugar comprising a bridge connecting two carbon atoms of the sugar ring one of which is the 2′ carbon. In some embodiments, a 2′-modification is non-bridging, e.g., allyl, amino, azido, thio, optionally substituted —O-allyl, optionally substituted —O—C₁-C₁₀ alkyl, —OCF₃, —O(CH₂)₂OCH₃, 2′-O(CH₂)₂SCH₃, —O(CH₂)₂ON(Rₘ)(Rₙ), or —OCH₂C(═O)N(Rₘ)(Rₙ), where each Rₘ and Rₙ is independently —H or optionally substituted C₁-C₁₀ alkyl.

In some embodiments, a sugar is the sugar of N-methanocarba, LNA, cMOE BNA, cEt BNA, α-L-LNA or related analogs, HNA, Me-ANA, MOE-ANA, Ara-FHNA, FHNA, R-6′-Me-FHNA, S-6′-Me-FHNA, ENA, or c-ANA. In some embodiments, a modified internucleotidic linkage is C3-amide (e.g., sugar that has the amide modification attached to the C3′, Mutisya et al. 2014 Nucleic Acids Res. 2014 Jun. 1; 42(10): 6542-6551), formacetal, thioformacetal, MMI [e.g., methylene(methylimino), Peoc'h et al. 2006 Nucleosides and Nucleotides 16 (7-9)], a PMO (phosphorodiamidate linked morpholino) linkage (which connects two sugars), or a PNA (peptide nucleic acid) linkage.

In some embodiments, a sugar is one described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, and/or WO 2020/191252, the sugars of each of which is incorporated herein by reference.

Various additional sugars useful for preparing oligonucleotides or analogs thereof are known in the art and may be utilized in accordance with the present disclosure.

### Nucleobases

Various nucleobases may be utilized in provided oligonucleotides in accordance with the present disclosure. In some embodiments, a nucleobase is a natural nucleobase, the most commonly occurring ones being A, T, C, G and U. In some embodiments, a nucleobase is a modified nucleobase in that it is not A, T, C, G or U. In some embodiments, a nucleobase is optionally substituted A, T, C, G or U, or a substituted tautomer of A T, C, G or U. In some embodiments, a nucleobase is optionally substituted A, T, C, G or U, e.g., 5mC, 5-hydroxymethyl C, etc. In some embodiments, a nucleobase is alkyl-substituted A, T, C, G or U. In some embodiments, a nucleobase is A. In some embodiments, a nucleobase is T. In some embodiments, a nucleobase is C. In some embodiments, a nucleobase is G. In some embodiments, a nucleobase is U. In some embodiments, a nucleobase is 5mC. In some embodiments, a nucleobase is substituted A, T, C, G or U. In some embodiments, a nucleobase is a substituted tautomer of A, T, C, G or U. In some embodiments, substitution protects certain functional groups in nucleobases to minimize undesired reactions during oligonucleotide synthesis. Suitable technologies for nucleobase protection in oligonucleotide synthesis are widely known in the art and may be utilized in accordance with the present disclosure. In some embodiments, modified nucleobases improves properties and/or activities of oligonucleotides. For example, in many cases, 5mC may be utilized in place of C to modulate certain undesired biological effects, e.g., immune responses. In some embodiments, when determining sequence identity, a substituted nucleobase having the same hydrogen-bonding pattern is treated as the same as the unsubstituted nucleobase, e.g., 5mC may be treated the same as C [e.g., an oligonucleotide having 5mC in place of C (e.g., AT5mCG) is considered to have the same base sequence as an oligonucleotide having C at the corresponding location(s) (e.g., ATCG)].

In some embodiments, an oligonucleotide comprises one or more A, T, C, G or U. In some embodiments, an oligonucleotide comprises one or more optionally substituted A, T, C, G or U. In some embodiments, an oligonucleotide comprises one or more 5-methylcytidine, 5-hydroxymethylcytidine, 5-formylcytosine, or 5-carboxylcytosine. In some embodiments, an oligonucleotide comprises one or more 5-methylcytidine. In some embodiments, each nucleobase in an oligonucleotide is selected from the group consisting of optionally substituted A, T, C, G and U, and optionally substituted tautomers of A, T, C, G and U. In some embodiments, each nucleobase in an oligonucleotide is optionally protected A, T, C, G and U. In some embodiments, each nucleobase in an oligonucleotide is optionally substituted A, T, C, G or U. In some embodiments, each nucleobase in an oligonucleotide is selected from the group consisting of A, T, C, G, U, and 5mC.

In some embodiments, a nucleobase is optionally substituted 2AP or DAP. In some embodiments, a nucleobase is optionally substituted 2AP. In some embodiments, a nucleobase is optionally substituted DAP. In some embodiments, a nucleobase is 2AP. In some embodiments, a nucleobase is DAP.

In some embodiments, a nucleobase is a natural nucleobase or a modified nucleobase derived from a natural nucleobase. Examples include uracil, thymine, adenine, cytosine, and guanine optionally having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). Certain examples of modified nucleobases are disclosed in Chiu and Rana, RNA, 2003, 9, 1034-1048, Limbach et al. Nucleic Acids Research, 1994, 22, 2183-2196 and Revankar and Rao, Comprehensive Natural Products Chemistry, vol. 7, 313. In some embodiments, a modified nucleobase is substituted uracil, thymine, adenine, cytosine, or guanine. In some embodiments, a modified nucleobase is a functional replacement, e.g., in terms of hydrogen bonding and/or base pairing, of uracil, thymine, adenine, cytosine, or guanine. In some embodiments, a nucleobase is optionally substituted uracil, thymine, adenine, cytosine, 5-methylcytosine, or guanine. In some embodiments, a nucleobase is uracil, thymine, adenine, cytosine, 5-methylcytosine, or guanine.

In some embodiments, a provided oligonucleotide comprises one or more 5-methylcytosine. In some embodiments, the present disclosure provides an oligonucleotide whose base sequence is disclosed herein, e.g., in Table 1, wherein each T may be independently replaced with U and vice versa, and each cytosine is optionally and independently replaced with 5-methylcytosine or vice versa. As appreciated by those skilled in the art, in some embodiments, 5mC may be treated as C with respect to base sequence of an oligonucleotide -such oligonucleotide comprises a nucleobase modification at the C position (e.g., see various oligonucleotides in Table 1). In description of oligonucleotides, typically unless otherwise noted, nucleobases, sugars and internucleotidic linkages are non-modified.

In some embodiments, a modified base is optionally substituted adenine, cytosine, guanine, thymine, or uracil, or a tautomer thereof. In some embodiments, a modified nucleobase is a modified adenine, cytosine, guanine, thymine or uracil, modified by one or more modifications by which:
(1) a nucleobase is modified by one or more optionally substituted groups independently selected from acyl, halogen, amino, azide, alkyl, alkenyl, alkynyl, aryl, heteroalkyl, heteroalkenyl, heteroalkynyl, heterocyclyl, heteroaryl, carboxyl, hydroxyl, biotin, avidin, streptavidin, substituted silyl, and combinations thereof;
(2) one or more atoms of a nucleobase are independently replaced with a different atom selected from carbon, nitrogen and sulfur;
(3) one or more double bonds in a nucleobase are independently hydrogenated; or
(4) one or more aryl or heteroaryl rings are independently inserted into a nucleobase.

In some embodiments, a modified nucleobase is a modified nucleobase known in the art, e.g., WO2017/210647. In some embodiments, modified nucleobases are expanded-size nucleobases in which one or more aryl and/or heteroaryl rings, such as phenyl rings, have been added.

In some embodiments, a modified nucleobase is selected from 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. In some embodiments, modified nucleobases are tricyclic pyrimidines, such as 1,3-diazaphenoxazine-2-one, 1,3-diazaphenothiazine-2-one or 9-(2-aminoethoxy)-1,3-diazaphenoxazine-2-one (G-clamp). In some embodiments, modified nucleobases are those in which the purine or pyrimidine base is replaced with other heterocycles, for example, 7-deaza-adenine, 7-deazaguanosine, 2-aminopyridine or 2-pyridone.

In some embodiments, a modified nucleobase is substituted. In some embodiments, a modified nucleobase is substituted such that it contains, e.g., heteroatoms, alkyl groups, or linking moieties connected to fluorescent moieties, biotin or avidin moieties, or other protein or peptides. In some embodiments, a modified nucleobase is a “universal base” that is not a nucleobase in the most classical sense, but that functions similarly to a nucleobase. One example of a universal base is 3-nitropyrrole.

In some embodiments, nucleosides that can be utilized in provided technologies comprise modified nucleobases and/or modified sugars, e.g., 4-acetylcytidine; 5-(carboxyhydroxylmethyl)uridine; 2′-O-methylcytidine; 5-carboxymethylaminomethyl-2-thiouridine; 5-carboxymethylaminomethyluridine; dihydrouridine; 2′-O-methylpseudouridine; beta,D-galactosylqueosine; 2′-O-methylguanosine; N⁶-isopentenyladenosine; 1-methyladenosine; 1-methylpseudouridine; 1-methylguanosine; 1-methylinosine; 2,2-dimethylguanosine; 2-methyladenosine; 2-methylguanosine; N⁷-methylguanosine; 3-methyl-cytidine; 5-methylcytidine; 5-hydroxymethylcytidine; 5-formylcytosine; 5-carboxylcytosine; N⁶-methyladenosine; 7-methylguanosine; 5-methylaminoethyluridine; 5-methoxyaminomethyl-2-thiouridine; beta,D-mannosylqueosine; 5-methoxycarbonylmethyluridine; 5-methoxyuridine; 2-methylthio-N⁶-isopentenyladenosine; N-((9-beta,D-ribofuranosyl-2-methylthiopurine-6-yl)carbamoyl)threonine; N-((9-beta,D-ribofuranosylpurine-6-yl)-N-methylcarbamoyl)threonine; uridine-5-oxyacetic acid methylester; uridine-5-oxyacetic acid (v); pseudouridine; queosine; 2-thiocytidine; 5-methyl-2-thiouridine; 2-thiouridine; 4-thiouridine; 5-methyluridine; 2′-O-methyl-5-methyluridine; and 2′-O-methyluridine.

In some embodiments, a nucleobase, e.g., a modified nucleobase comprises one or more biomolecule binding moieties such as e.g., antibodies, antibody fragments, biotin, avidin, streptavidin, receptor ligands, or chelating moieties. In other embodiments, a nucleobase is 5-bromouracil, 5-iodouracil, or 2,6-diaminopurine. In some embodiments, a nucleobase comprises substitution with a fluorescent or biomolecule binding moiety. In some embodiments, a substituent is a fluorescent moiety. In some embodiments, a substituent is biotin or avidin.

In some embodiments, a nucleobase is one described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, and/or WO 2020/191252, the nucleobases of each of which is incorporated herein by reference.

### Additional Chemical Moieties

In some embodiments, a ds oligonucleotide targeting HSD17B13 comprises one or more additional chemical moieties. Various additional chemical moieties, e.g., targeting moieties, carbohydrate moieties, lipid moieties, etc. are known in the art and can be utilized in accordance with the present disclosure to modulate properties and/or activities of ds oligonucleotides targeting HSD17B13, e.g., stability, half life, activities, delivery, pharmacodynamics properties, pharmacokinetic properties, etc. In some embodiments, certain additional chemical moieties facilitate delivery of oligonucleotides to desired cells, tissues and/or organs, including but not limited the cells of the central nervous system. In some embodiments, certain additional chemical moieties facilitate internalization of oligonucleotides. In some embodiments, certain additional chemical moieties increase oligonucleotide stability. In some embodiments, the present disclosure provides technologies for incorporating various additional chemical moieties into oligonucleotides.

In certain embodiments, a ds oligonucleotide comprises an additional chemical moiety demonstrates increased delivery to and/or activity in a tissue compared to a reference oligonucleotide, e.g., a reference oligonucleotide which does not have the additional chemical moiety but is otherwise identical.

In certain embodiments, non-limiting examples of additional chemical moieties include carbohydrate moieties, targeting moieties, etc., which, when incorporated into oligonucleotides, can improve one or more properties. In certain embodiments, an additional chemical moiety is selected from: glucose, GluNAc (N-acetyl amine glucosamine) and anisamide moieties. In certain embodiments, a provided ds oligonucleotide can comprise two or more additional chemical moieties, wherein the additional chemical moieties are identical or non-identical, or are of the same category (e.g., carbohydrate moiety, sugar moiety, targeting moiety, etc.) or not of the same category.

In certain embodiments, an additional chemical moiety is a targeting moiety. In certain embodiments, an additional chemical moiety is or comprises a carbohydrate moiety. In certain embodiments, an additional chemical moiety is or comprises a lipid moiety. In certain embodiments, an additional chemical moiety is or comprises a ligand moiety for, e.g., cell receptors such as a sigma receptor, an asialoglycoprotein receptor, etc. In certain embodiments, a ligand moiety is or comprises an anisamide moiety, which may be a ligand moiety for a sigma receptor. In certain embodiments, a ligand moiety is or comprises a GalNAc moiety, which may be a ligand moiety for an asialoglycoprotein receptor. In certain embodiments, an additional chemical moiety facilitates delivery to liver.

In certain embodiments, a provided ds oligonucleotide can comprise one or more linkers and additional chemical moieties (e.g., targeting moieties), and/or can be chirally controlled or not chirally controlled, and/or have a bases sequence and/or one or more modifications and/or formats as described herein.

Various linkers, carbohydrate moieties and targeting moieties, including many known in the art, can be utilized in accordance with the present disclosure. In certain embodiments, a carbohydrate moiety is a targeting moiety. In certain embodiments, a targeting moiety is a carbohydrate moiety. In certain embodiments, a provided ds oligonucleotide comprises an additional chemical moiety suitable for delivery, e.g., glucose, GluNAc (N-acetyl amine glucosamine), anisamiide, or a structure selected from:

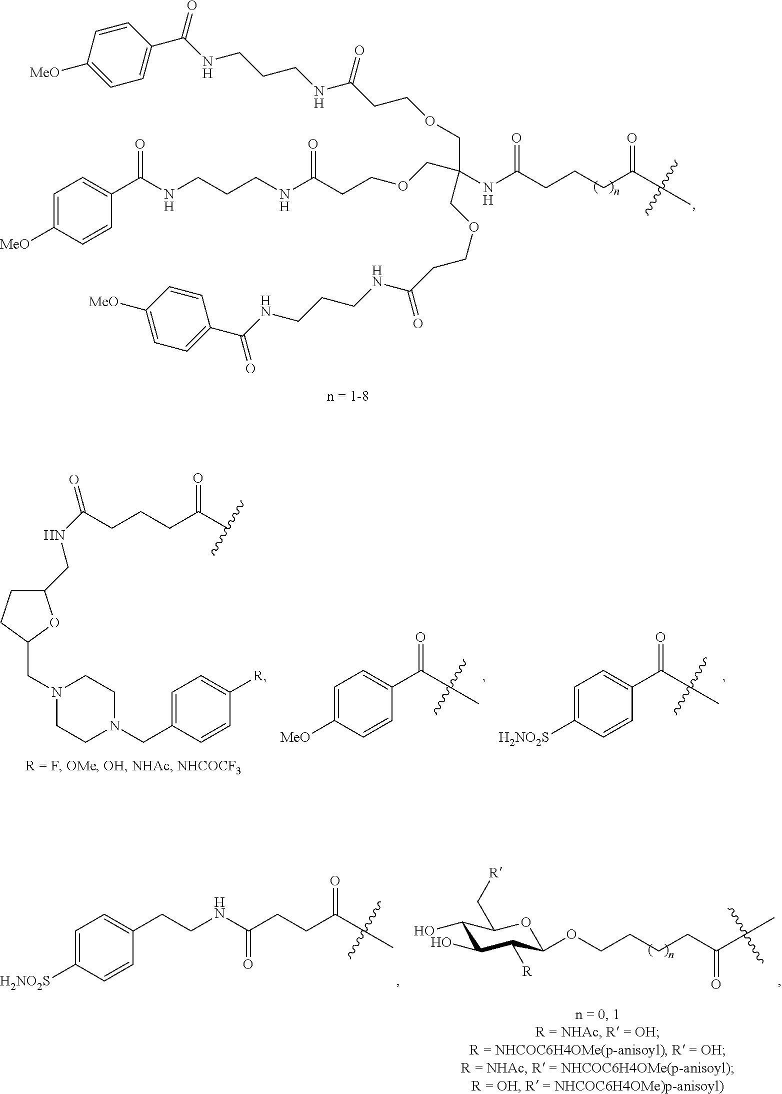

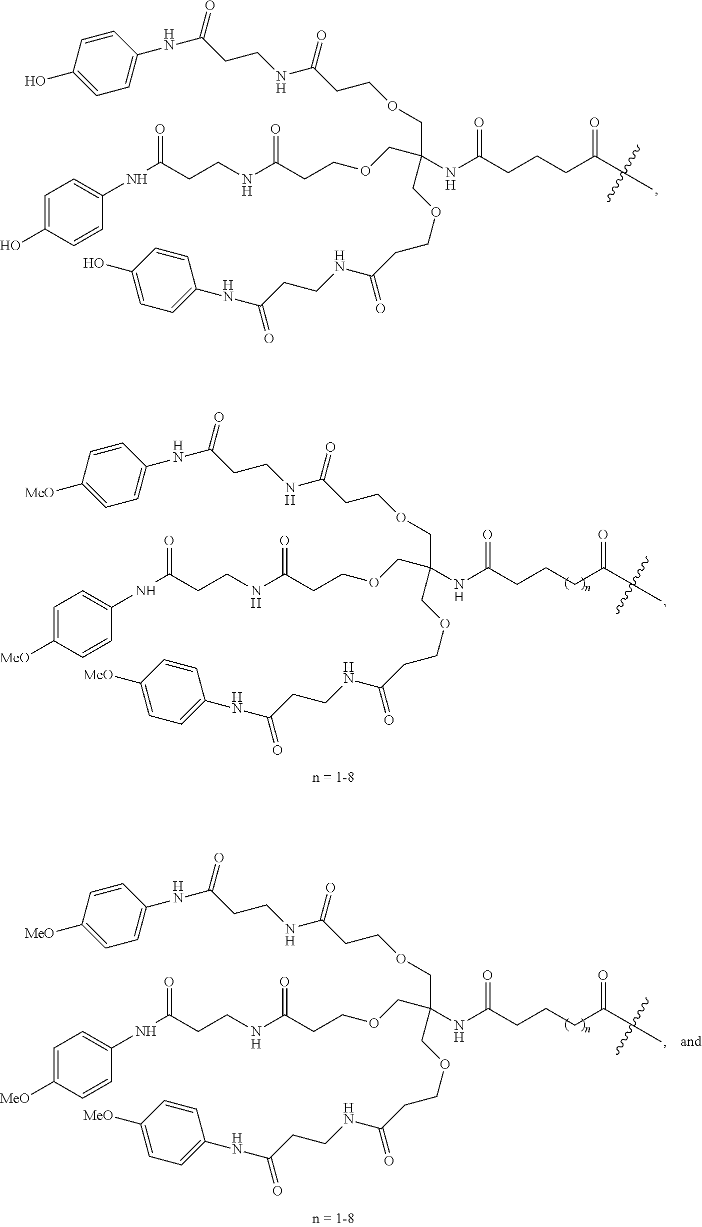

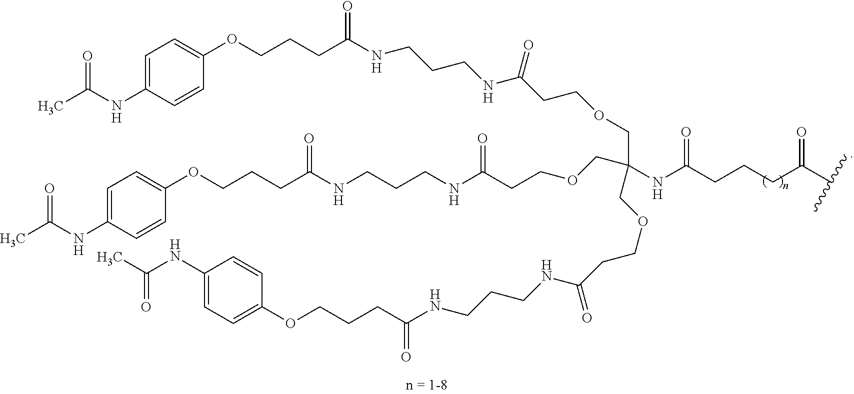

In certain embodiments, n is 1. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8.

In certain embodiments, additional chemical moieties are any of ones described in the Examples, including examples of various additional chemical moieties incorporated into various ds oligonucleotides.

In certain embodiments, an additional chemical moiety conjugated to a ds oligonucleotide is capable of targeting the ds oligonucleotide to a cell in the central nervous system.

In certain embodiments, an additional chemical moiety comprises or is a cell receptor ligand. In certain embodiments, an additional chemical moiety comprises or is a protein binder, e.g., one binds to a cell surface protein. Such moieties among other things can be useful for targeted delivery of ds oligonucleotides to cells expressing the corresponding receptors or proteins. In certain embodiments, an additional chemical moiety of a provided ds oligonucleotide comprises anisamide or a derivative or an analog thereof and is capable of targeting the ds oligonucleotide to a cell expressing a particular receptor, such as the sigma 1 receptor.

In certain embodiments, a provided ds oligonucleotide is formulated for administration to a body cell and/or tissue expressing its target. In certain embodiments, an additional chemical moiety conjugated to a ds oligonucleotide is capable of targeting the oligonucleotide to a cell.

In certain embodiments, an additional chemical moiety is selected from optionally substituted phenyl,

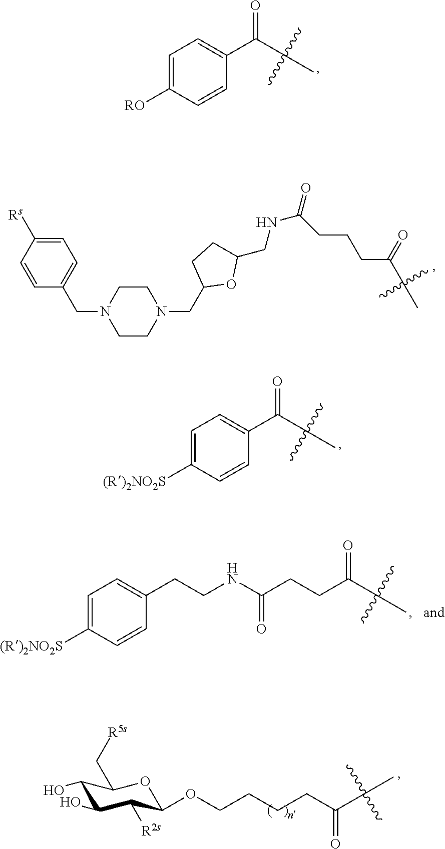

wherein n′ is 1, 2, 3, 4, 5, 6, 7, 8, 9, or 10, and each other variable is as described in the present disclosure. In certain embodiments, R^{s} is F. In certain embodiments, R^{s} is OMe. In certain embodiments, R^{s} is OH. In certain embodiments, R^{s} is NHAc. In certain embodiments, R^{s} is NHCOCF₃. In certain embodiments, R′ is H. In certain embodiments, R is H. In certain embodiments, R^{2s} is NHAc, and R^{5s} is OH. In certain embodiments, R^{2s} is p-anisoyl, and R^{5s} is OH. In certain embodiments, R^{2s} is NHAc and R^{5s} is p-anisoyl. In certain embodiments, R^{2s} is OH, and R^{5s} is p-anisoyl. In certain embodiments, an additional chemical moiety is selected from

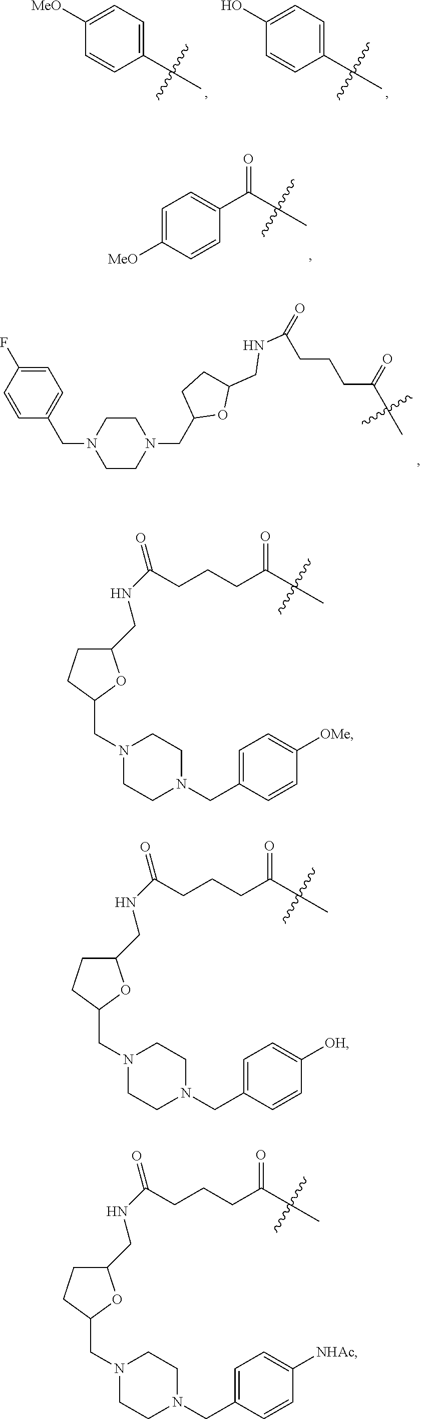

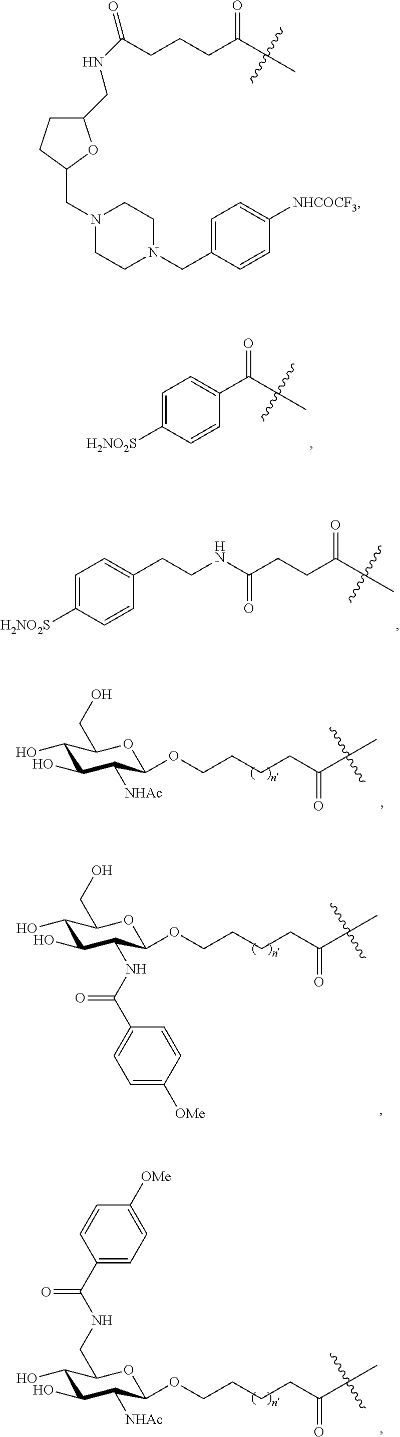

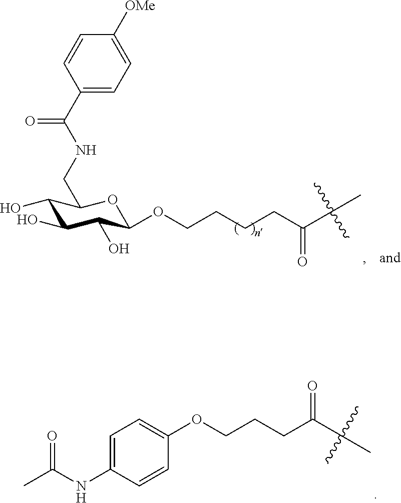

In certain embodiments, n′ is 1. In certain embodiments, n′ is 0. In certain embodiments, n″ is 1. In certain embodiments, n″ is 2.

In certain embodiments, an additional chemical moiety is or comprises an asialoglycoprotein receptor (ASGPR) ligand.

Without wishing to be bound by any particular theory, the present disclosure notes that ASGPR1 has also been reported to be expressed in the hippocampus region and/or cerebellum Purkinje cell layer of the mouse. http://mouse.brain-map.org/experiment/show/2048

Various other ASGPR ligands are known in the art and can be utilized in accordance with the present disclosure. In certain embodiments, an ASGPR ligand is a carbohydrate. In certain embodiments, an ASGPR ligand is GalNac or a derivative or an analog thereof. In certain embodiments, an ASGPR ligand is one described in Sanhueza et al. J. Am. Chem. Soc., 2017, 139 (9), pp 3528-3536. In certain embodiments, an ASGPR ligand is one described in Mamidyala et al. J. Am. Chem. Soc., 2012, 134, pp 1978-1981. In certain embodiments, an ASGPR ligand is one described in US 20160207953. In certain embodiments, an ASGPR ligand is a substituted-6,8-dioxabicyclo[3.2.1]octane-2,3-diol derivative disclosed in, e.g., US 20160207953. In certain embodiments, an ASGPR ligand is one described in, e.g., US 20150329555. In certain embodiments, an ASGPR ligand is a substituted-6,8-dioxabicyclo[3.2.1]octane-2,3-diol derivative disclosed e.g., in US 20150329555. In certain embodiments, an ASGPR ligand is one described in U.S. Pat. No. 8,877,917, US 20160376585, U.S. Ser. No. 10/086,081, or U.S. Pat. No. 8,106,022. ASGPR ligands described in these documents are incorporated herein by reference. Those skilled in the art will appreciate that various technologies are known in the art, including those described in these documents, for assessing binding of a chemical moiety to ASGPR and can be utilized in accordance with the present disclosure. In certain embodiments, a provided ds oligonucleotide is conjugated to an ASGPR ligand. In certain embodiments, a provided ds oligonucleotide comprises an ASGPR ligand. In certain embodiments, an additional chemical moiety comprises an ASGPR ligand is

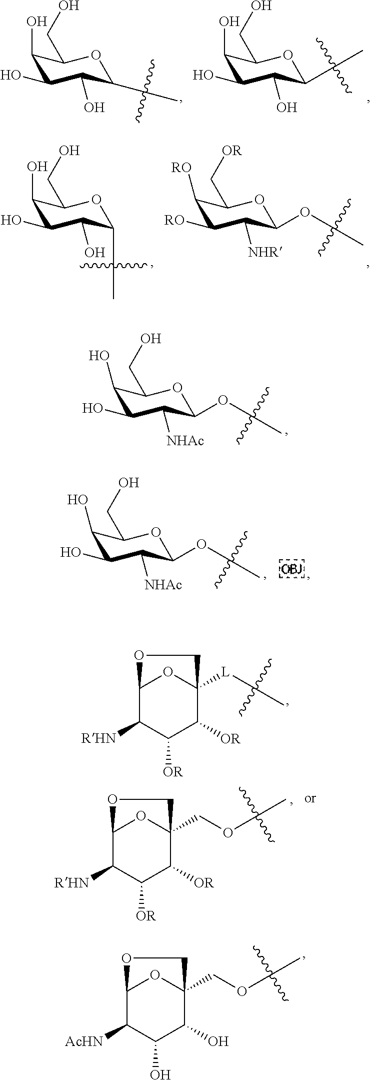

wherein each variable is independently as described in the present disclosure. In certain embodiments, R is —H. In certain embodiments, R′ is —C(O)R.

In certain embodiments, an additional chemical moiety is or comprises

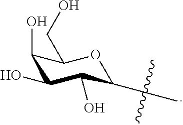

In certain embodiments, an additional chemical moiety is or comprises

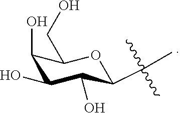

In certain embodiments, an additional chemical moiety is or comprises

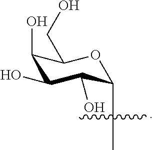

In certain embodiments, an additional chemical moiety is or comprises

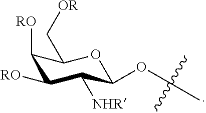

In certain embodiments, an additional chemical moiety is or comprises optionally substituted

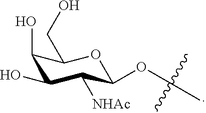

In certain embodiments, an additional chemical moiety is or comprises

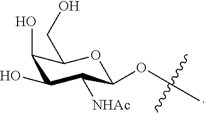

In certain embodiments, an additional chemical moiety is or comprises [symbol] . In certain embodiments, an additional chemical moiety is or comprises

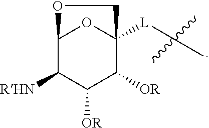

In certain embodiments, an additional chemical moiety is or comprises

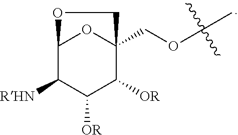

In certain embodiments, an additional chemical moiety is or comprises

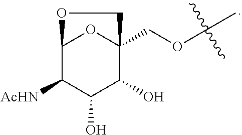 [SMILES: *OC[C@@]12CO[C@@H](O1)[C@H](NC(C)=O)[C@@H](O)[C@H]2O]

In certain embodiments, an additional chemical moiety comprises one or more moieties that can bind to, e.g., oligonucleotide target cells. For example, in certain embodiments, an additional chemistry moiety comprises one or more protein ligand moieties, e.g., in certain embodiments, an additional chemical moiety comprises multiple moieties, each of which independently is an ASGPR ligand. In certain embodiments, as in Mod001, Mod083, Mod071, Mod153, and Mod155, an additional chemical moiety comprises three such ligands.

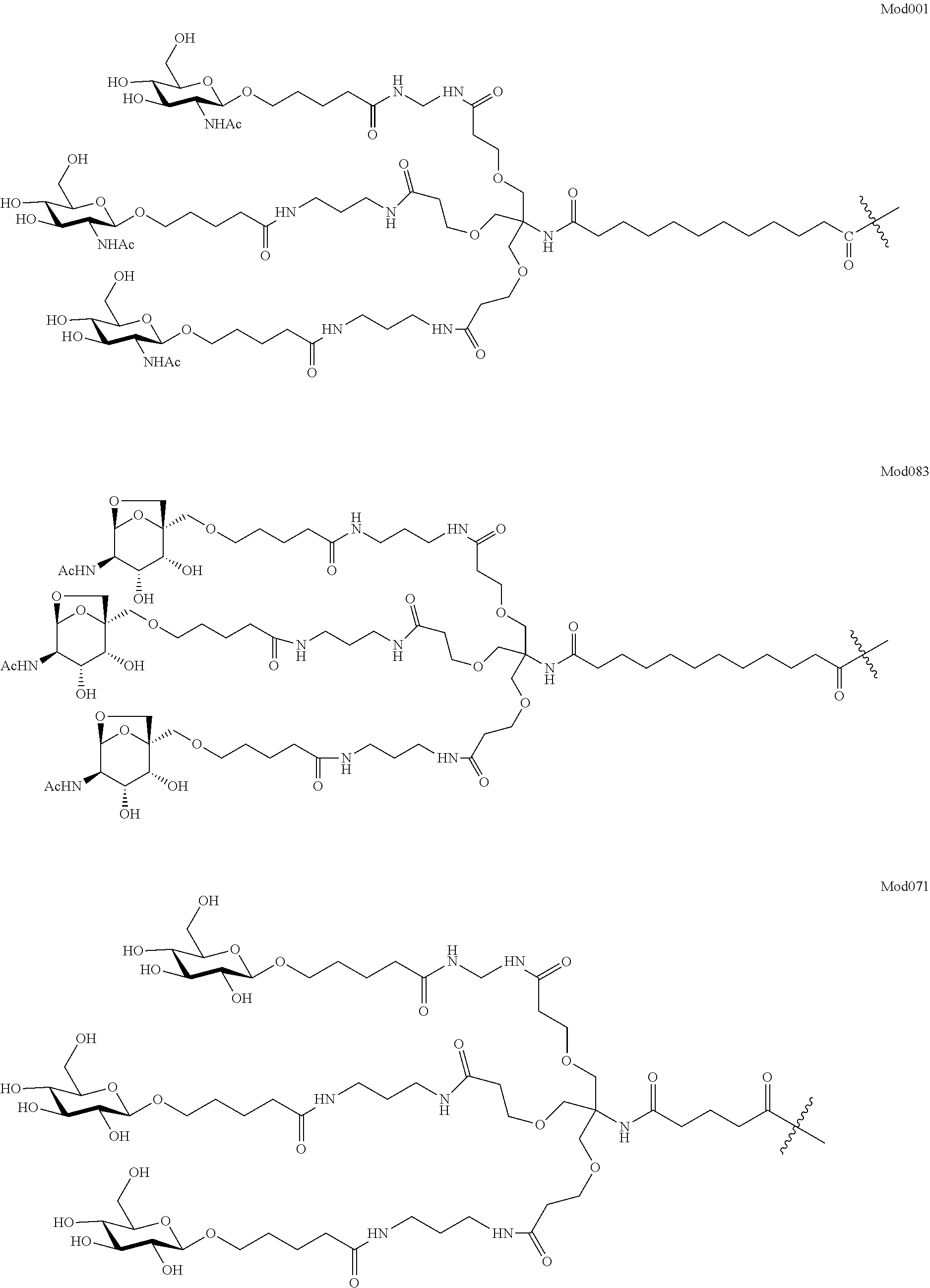

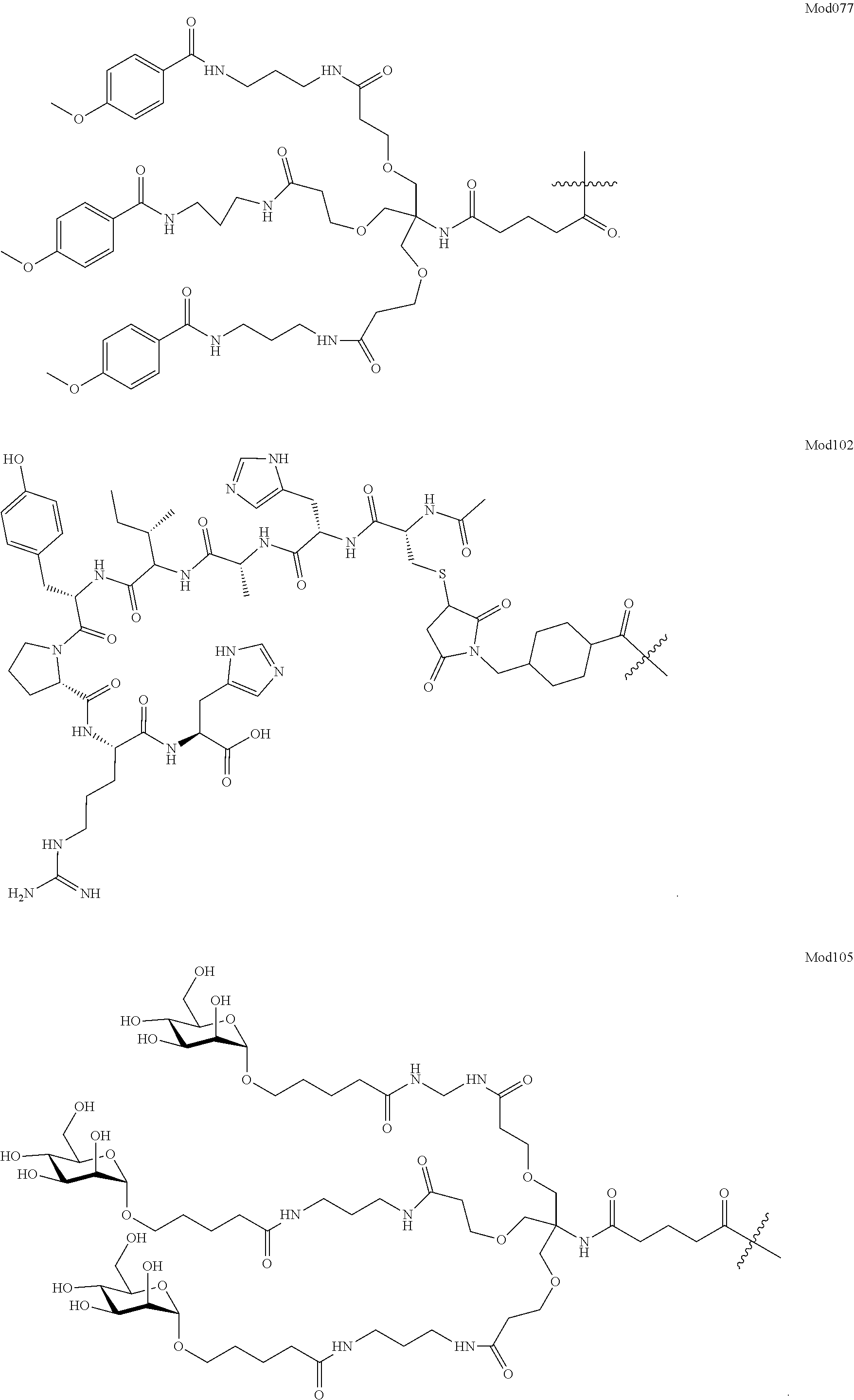

Mod152 (in certain embodiments, —C(O)— connects to —NH— of a linker such as Mod153):

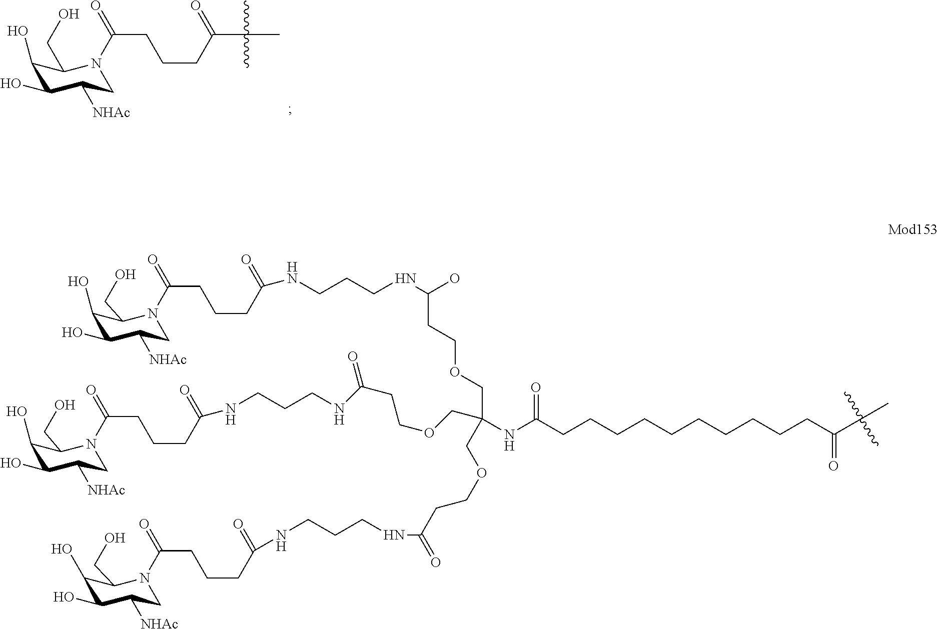

Mod154 (in certain embodiments, —C(O)— connects to —NH— of a linker such as Mod155):

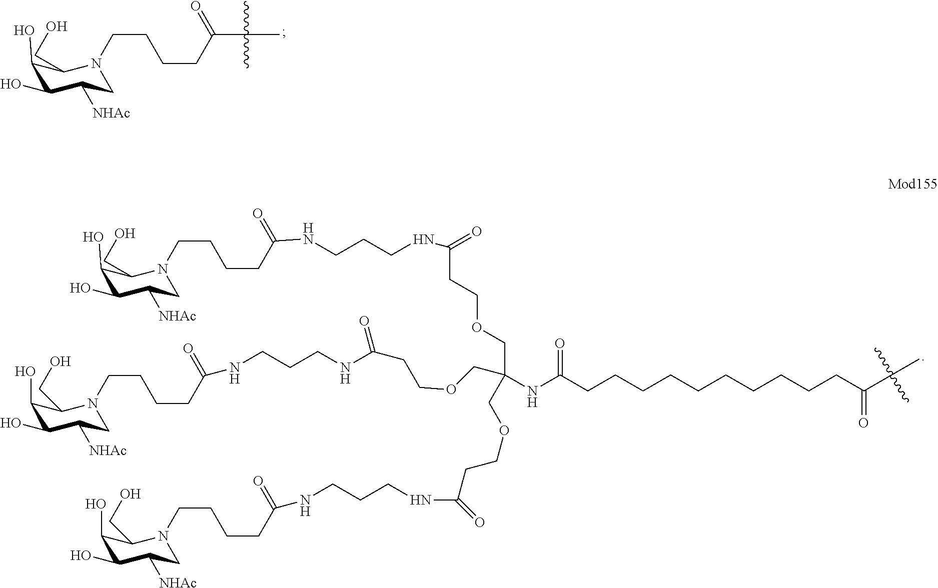

In some embodiments, an oligonucleotide comprises

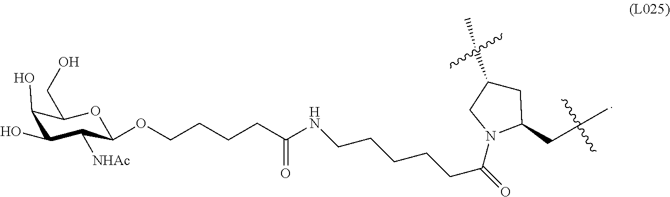

In some embodiments, the —CH₂— connection site is utilized as a C5 connection site in a sugar. In some embodiments, the connection site on the ring is utilized as a C3 connection site in a sugar. Such moieties may be introduced utilizing, e.g., phosphoramidites (those skilled in the art appreciate that one or more groups, such as protection groups for —OH, —NH₂—, —N(i-Pr)₂, —OCH₂CH₂CN, etc., may be alternatively utilized, and protection groups can be removed under various suitable conditions, sometimes during oligonucleotide de-protection and/or cleavage steps). In some embodiments, an oligonucleotide comprises 2, 3 or more (e.g., 3 and no more than 3). In some embodiments, an oligonucleotide comprises 2, 3 or more (e.g., 3 and no more than 3). In some embodiments, copies of such moieties are linked by internucleotidic linkages, e.g., natural phosphate linkages, as described herein. In some embodiments, when at a 5′-end, a —CH₂-connection site is bonded to —OH. In some embodiments, each —OR′ is —OAc, and —N(R′)₂ is —NHAc.

In certain embodiments, an additional chemical moiety is a Mod group described herein, e.g., in Table 1.

In certain embodiments, an additional chemical moiety is Mod001. In certain embodiments, an additional chemical moiety is Mod083. In certain embodiments, an additional chemical moiety, e.g., a Mod group, is directly conjugated (e.g., without a linker) to the remainder of the ds oligonucleotide. In certain embodiments, an additional chemical moiety is conjugated via a linker to the remainder of the ds oligonucleotide. In certain embodiments, additional chemical moieties, e.g., Mod groups, may be directly connected, and/or via a linker, to nucleobases, sugars and/or internucleotidic linkages of ds oligonucleotides. In certain embodiments, Mod groups are connected, either directly or via a linker, to sugars. In certain embodiments, Mod groups are connected, either directly or via a linker, to 5′-end sugars. In certain embodiments, Mod groups are connected, either directly or via a linker, to 5′-end sugars via 5′ carbon. For examples, see various ds oligonucleotides in Table 1. In certain embodiments, Mod groups are connected, either directly or via a linker, to 3′-end sugars. In certain embodiments, Mod groups are connected, either directly or via a linker, to 3′-end sugars via 3′ carbon. In certain embodiments, Mod groups are connected, either directly or via a linker, to nucleobases. In certain embodiments, Mod groups are connected, either directly or via a linker, to internucleotidic linkages. In certain embodiments, provided oligonucleotides comprise Mod001 connected to 5′-end of oligonucleotide chains through L001.

As appreciated by those skilled in the art, an additional chemical moiety may be connected to a ds oligonucleotide chain at various locations, e.g., 5′-end, 3′-end, or a location in the middle (e.g., on a sugar, a base, an internucleotidic linkage, etc.). In certain embodiments, it is connected at a 5′-end. In certain embodiments, it is connected at a 3′-end. In certain embodiments, it is connected at a nucleotide in the middle.

Certain additional chemical moieties (e.g., lipid moieties, targeting moieties, carbohydrate moieties), including but not limited to Mod012, Mod039, Mod062, Mod085, Mod086, and Mod094, and various linkers for connecting additional chemical moieties to ds oligonucleotide chains, including but not limited to L001, L003, L004, L008, L009, and L010, are described in WO 2017/062862, WO 2018/067973, WO 2017/160741, WO 2017/192679, WO 2017/210647, WO 2018/098264, WO 2018/022473, WO 2018/223056, WO 2018/223073, WO 2018/223081, WO 2018/237194, WO 2019/032607, WO2019032612, WO 2019/055951, WO 2019/075357, WO 2019/200185, WO 2019/217784, and/or WO 2019/032612, the additional chemical moieties and linkers of each of which are independently incorporated herein by reference, and can be utilized in accordance with the present disclosure. In certain embodiments, an additional chemical moiety is digoxigenin or biotin or a derivative thereof.

In certain embodiments, a ds oligonucleotide comprises a linker, e.g., L001 L004, L008, and/or an additional chemical moiety, e.g., Mod012, Mod039, Mod062, Mod085, Mod086, or Mod094. In certain embodiments, a linker, e.g., L001, L003, L004, L008, L009, L110, etc. is linked to a Mod, e.g., Mod012, Mod039, Mod062, Mod085, Mod086, Mod094, Mod152, Mod153, Mod154, Mod155 etc. L001: —NH—(CH₂)₆— linker (also known as a C6 linker, C6 amine linker or C₆ amino linker), connected to Mod, if any, through —NH—, and the 5′-end or 3′-end of the ds oligonucleotide chain through either a phosphate linkage (—O—P(O)(OH)—O—, which may exist as a salt form, and may be indicated as O or PO) or a phosphorothioate linkage (—O—P(O)(SH)—O—, which may exist as a salt form, and may be indicated as * if the phosphorothioate is not chirally controlled; or *S, S, or Sp, if the phosphorothioate is chirally controlled and has an Sp configuration, or *R, R, or Rp, if the phosphorothioate is chirally controlled and has an Rp configuration) as indicated at the —CH₂— connecting site. If no Mod is present, L001 is connected to —H through —NH—;
L003:

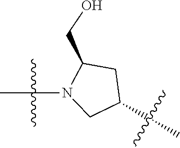 [SMILES: *[C@H]1C[C@H](CO)N(*)C1]

linker. In certain embodiments, it is connected to Mod, if any (if no Mod, —H), through its amino group, and the 5′-end or 3′-end of an oligonucleotide chain e.g., via a linkage (e.g., a phosphate linkage (O or PO) or a phosphorothioate linkage (can be either not chirally controlled or chirally controlled (Sp or Rp))); L004: linker having the structure of —NH(CH₂)₄CH(CH₂OH)CH₂—, wherein —NH— is connected to Mod (through —C(O)—) or —H, and the —CH₂— connecting site is connected to an oligonucleotide chain (e.g., at the 3′-end) through a linkage, e.g., phosphodiester (—O—P(O)(OH)—O—, which may exist as a salt form, and may be indicated as O or PO), phosphorothioate (—O—P(O)(SH)—O—, which may exist as a salt form, and may be indicated as * if the phosphorothioate is not chirally controlled; or *S, S, or Sp, if the phosphorothioate is chirally controlled and has an Sp configuration, or *R, R, or Rp, if the phosphorothioate is chirally controlled and has an Rp configuration), or phosphorodithioate (—O—P(S)(SH)—O—, which may exist as a salt form, and may be indicated as PS2 or: or D) linkage. For example, an asterisk immediately preceding a L004 (e.g., *L004) indicates that the linkage is a phosphorothioate linkage, and the absence of an asterisk immediately preceding L004 indicates that the linkage is a phosphodiester linkage. For example, in an oligonucleotide which terminates in . . . mAL004, the linker L004 is connected (via the —CH₂— site) through a phosphodiester linkage to the 3′ position of the 3′-terminal sugar (which is 2′-OMe modified and connected to the nucleobase A), and the L004 linker is connected via —NH— to —H. Similarly, in one or more oligonucleotides, the L004 linker is connected (via the —CH₂— site) through the phosphodiester linkage to the 3′ position of the 3′-terminal sugar, and the L004 is connected via —NH— to, e.g., Mod012, Mod085, Mod086, etc.; L008: linker having the structure of —C(O)—(CH₂)₉—, wherein —C(O)— is connected to Mod (through —NH—) or —OH (if no Mod indicated), and the —CH₂— connecting site is connected to an oligonucleotide chain (e.g., at the 5′-end) through a linkage, e.g., phosphodiester (—O—P(O)(OH)—O—, which may exist as a salt form, and may be indicated as O or PO), phosphorothioate (—O—P(O)(SH)—O—, which may exist as a salt form, and may be indicated as * if the phosphorothioate is not chirally controlled; or *S, S, or Sp, if the phosphorothioate is chirally controlled and has an Sp configuration, or *R, R, or Rp, if the phosphorothioate is chirally controlled and has an Rp configuration), or phosphorodithioate (—O—P(S)(SH)—O—, which may exist as a salt form, and may be indicated as PS2 or: or D) linkage. For example, in an example oligonucleotide which has the sequence of 5′-L008 mN * mN * mN * mN * N * N * N * N * N * N * N * N * N * N * mN * mN * mN * mN-3′, and which has a Stereochemistry/Linkage of OXXXXXXXXX XXXXXXXX, wherein N is a base, wherein O is a natural phosphate internucleotidic linkage, and wherein X is a stereorandom phosphorothioate, L008 is connected to —OH through —C(O)—, and the 5′-end of an oligonucleotide chain through a phosphate linkage (indicated as “0” in “Stereochemistry/Linkage”); in another example oligonucleotide, which has the sequence of 5′-Mod062L008 mN * mN* mN * mN * N * N * N * N * N * N * N * N * N * N * N * mN * mN * mN-3′, and which has a Stereochemistry/Linkage of OXXXXXXXXXXXXXXXXX, wherein N is a base, L008 is connected to Mod062 through —C(O)—, and the 5′-end of an oligonucleotide chain through a phosphate linkage (indicated as “0” in “Stereochemistry/Linkage”);
L009: —CH₂CH₂CH₂—. In certain embodiments, when L009 is present at the 5′-end of an oligonucleotide without a Mod, one end of L009 is connected to —OH and the other end connected to a 5′-carbon of the oligonucleotide chain e.g., via a linkage (e.g., a phosphate linkage (O or PO) or a phosphorothioate linkage (can be either not chirally controlled or chirally controlled (Sp or Rp))); L0110:

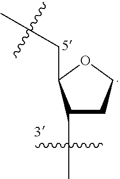

In certain embodiments, when L010 is present at the 5′-end of an oligonucleotide without a Mod, the 5′-carbon of LOW is connected to —OH and the 3′-carbon connected to a 5′-carbon of the oligonucleotide chain e.g., via a linkage (e.g., a phosphate linkage (O or PO) or a phosphorothioate linkage (can be either not chirally controlled or chirally controlled (Sp or Rp))); Mod012 (in certain embodiments, —C(O)-connects to —NH— of a linker such as L001, L004, L008, etc.):
L010 is utilized with n001R to form L010n001R, which has the structure of

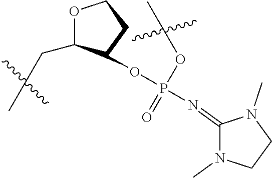

and wherein the configuration of linkage phosphorus is Rp. In some embodiments, multiple L010n001R may be utilized. For example, L023L010n001RL010n001RL010n001R, which has the following structure (which is bonded to the 5′-carbon at the 5′-end of the oligonucleotide chain, and each linkage phosphorus is independently Rp):

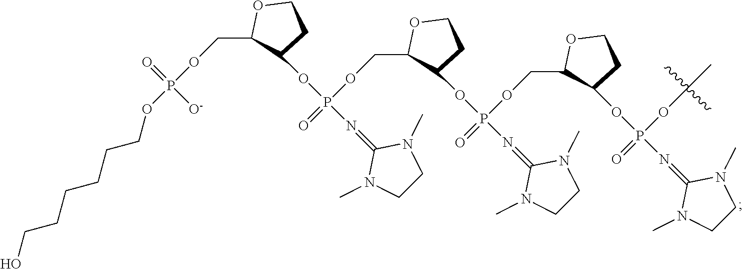

L023 is utilized with n001 to form L023n001, which has the structure of

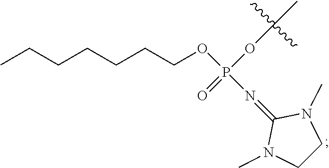 [SMILES: *OP(=O)(N=C1N(C)CCN1C)OCCCCCCC]

L023 is utilized with n009 to form L023n009, as in WV-42644 which has the structure of

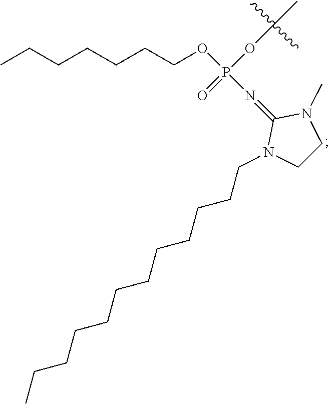 [SMILES: *OP(=O)(N=C1N(C)CCN1CCCCCCCCCCCC)OCCCCCCC]

In some embodiments, L023n001L009n001L009n001 may be utilized. For example, L023n001L009n001L009n001 as in WV-42643

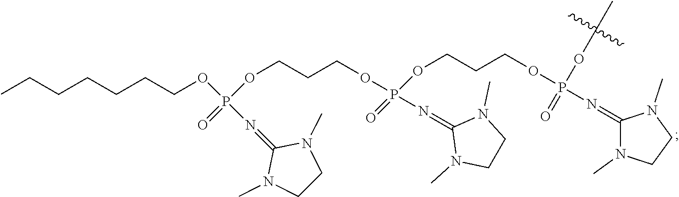 [SMILES: *OP(=O)(N=C1N(C)CCN1C)OCCCOP(=O)(N=C1N(C)CCN1C)OCCCOP(=O)(N=C1N(C)CCN1C)OCCCCCCC]

In some embodiments, L023n009L009n009 may be utilized. For example, as in WV-42646

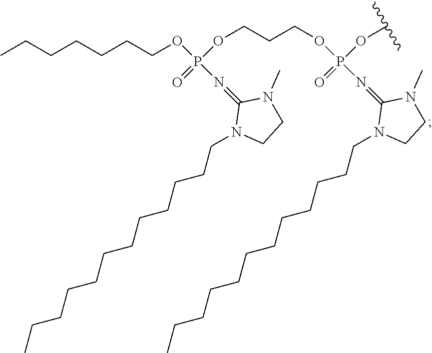 [SMILES: *OP(=O)(N=C1N(C)CCN1CCCCCCCCCCCC)OCCCOP(=O)(N=C1N(C)CCN1CCCCCCCCCCCC)OCCCCCCC]

In some embodiments, L023n009L009n009L009n009 may be utilized. For example, as in WV-42648

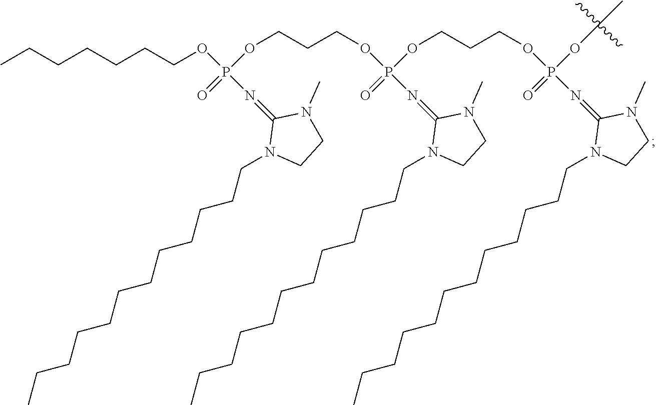 [SMILES: *OP(=O)(N=C1N(C)CCN1CCCCCCCCCCCC)OCCCOP(=O)(N=C1N(C)CCN1CCCCCCCCCCCC)OCCCOP(=O)(N=C1N(C)CCN1CCCCCCCCCCCC)OCCCCCCC]

In some embodiments L025 may be utilized; as in WV-41390,

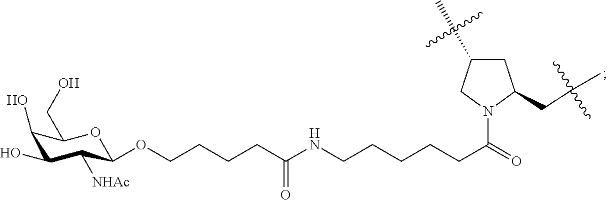

wherein the —CH₂— connection site is utilized as a C₅ connection site of a sugar (e.g., a DNA sugar) and is connected to another unit (e.g., 3′ of a sugar), and the connection site on the ring is utilized as a C₃ connection site and is connected to another unit (e.g., a 5′-carbon of a carbon), each of which is independently, e.g., via a linkage (e.g., a phosphate linkage (O or PO) or a phosphorothioate linkage (can be either not chirally controlled or chirally controlled (Sp or Rp))). When L025 is at a5′-end without any modifications, its —CH₂— connection site is bonded to —OH. For example, L025L025L025—in various oligonucleotides has the structure of

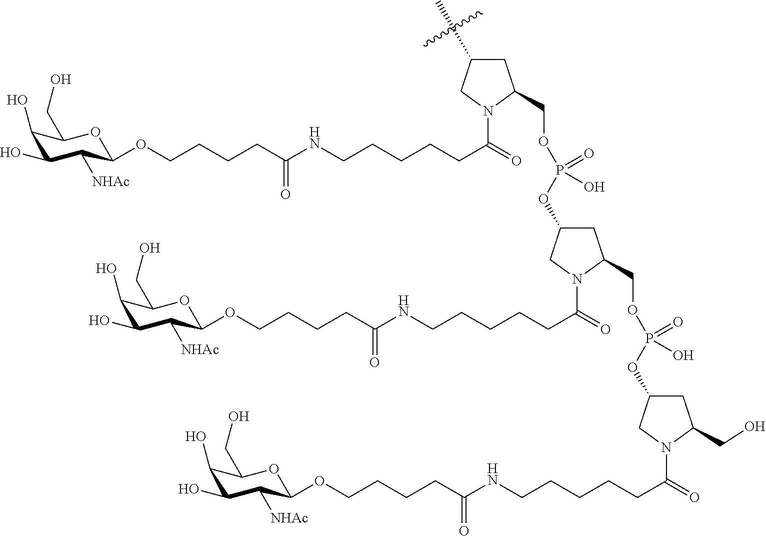

(may exist as various salt forms) and is connected to 5′-carbon of an oligonucleotide chain via a linkage as indicated (e.g., a phosphate linkage (O or PO) or a phosphorothioate linkage (can be either not chirally controlled or chirally controlled (Sp or Rp)));

In some embodiments L026 may be utilized; as in WV-44444,

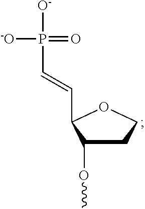

In some embodiments L027 may be utilized; as in WV-44445,

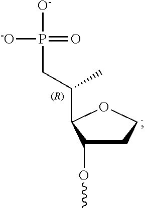

In some embodiments mU may be utilized; as in WV-42079,

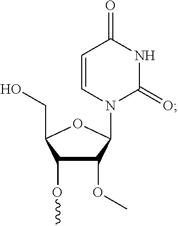

In some embodiments fU may be utilized; as in WV-44433,

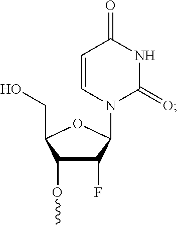

In some embodiments dT may be utilized; as in WV-44434,

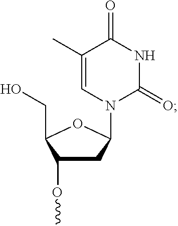

In some embodiments POdT or PO4-dT may be utilized; as in WV-44435,

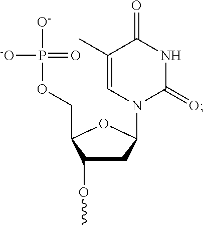

In some embodiments PO5MRdT may be utilized; as in WV-44436,

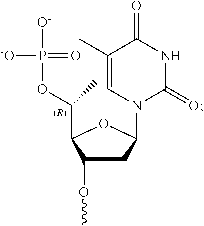

In some embodiments PO5MSdT may be utilized; as in WV-44437,

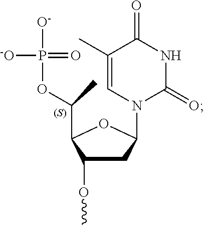

In some embodiments VPdT may be utilized; as in WV-44438,

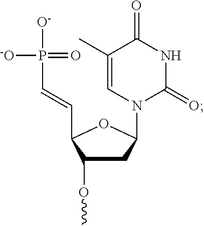

In some embodiments 5mvpdT may be utilized; as in WV-44439,

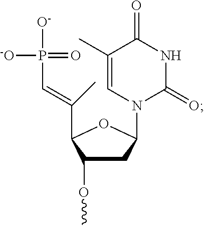

In some embodiments 5mrpdT may be utilized; as in WV-44440,

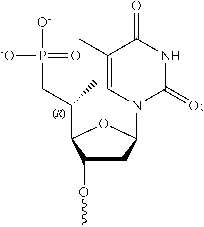

In some embodiments 5mspdT may be utilized; as in WV-44441,

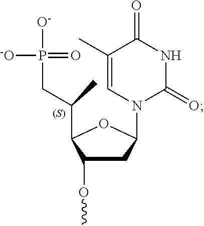

In some embodiments PNdT may be utilized; as in WV-44442,

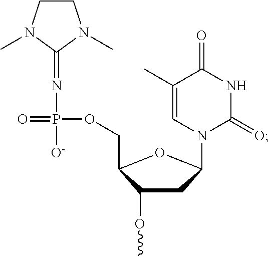

In some embodiments SPNdT may be utilized; as in WV-44443,

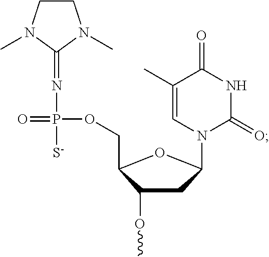

In some embodiments 5ptzdT may be utilized; as in WV-44446,

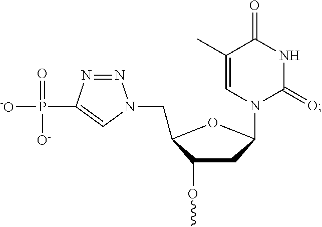

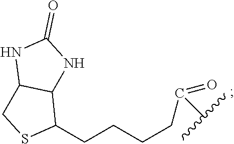 [SMILES: *C(=O)CCCCC1SCC2NC(=O)NC21]

Mod039 (in certain embodiments, —C(O)— connects to —NH— of a linker such as L001, L003, L004, L008, L009, L110, etc.):

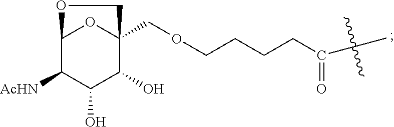 [SMILES: *C(=O)CCCCOC[C@@]12CO[C@@H](O1)[C@H](NC(C)=O)[C@@H](O)[C@H]2O]

Mod062 (in certain embodiments, —C(O)— connects to —NH— of a linker such as L001, L003, L004, L008, L009, L110, etc.):

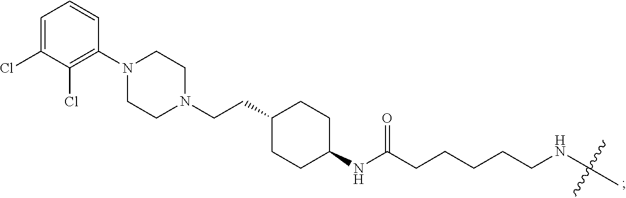 [SMILES: *NCCCCCC(=O)N[C@H]1CC[C@H](CCN2CCN(c3cccc(Cl)c3Cl)CC2)CC1]

Mod085 (in certain embodiments, —C(O)— connects to —NH— of a linker such as L001, L003, L004, L008, L009, L110, etc.):

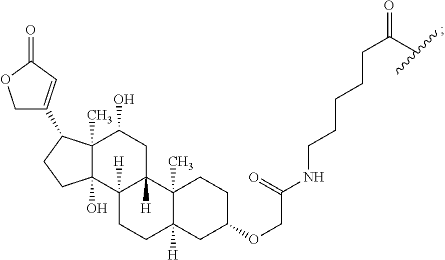

Mod086 (in certain embodiments, —C(O)-connects to —NH— of a linker such as L001, L003, L004, L008, L009, L110, etc.):

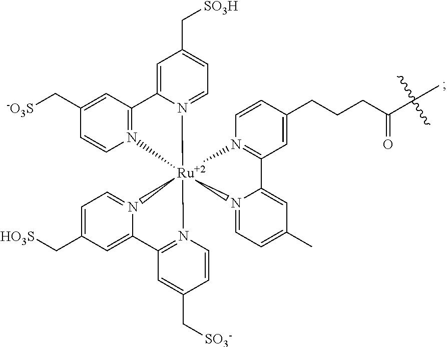

Mod094 (in certain embodiments, connects to an internucleotidic linkage, or to the 5′-end or 3′-end of an oligonucleotide via a linkage, e.g., a phosphate linkage, a phosphorothioate linkage (which is optionally chirally controlled), etc. For example, in an example oligonucleotide which has the sequence of 5′-mN * mN * mN * mN* N * N * N * N * N * N * N * N * N * N * mN * mN* mN * mNMod094-3′, and which has a Stereochemistry/Linkage of XXXXX XXXXX XXXXX XXO, wherein N is a base, Mod094 is connected to the 3′-end of the oligonucleotide chain (3′-carbon of the 3′-end sugar) through a phosphate group (which is not shown below and which may exist as a salt form; and which is indicated as “0” in “Stereochemistry/Linkage” ( . . . XXXXO))):

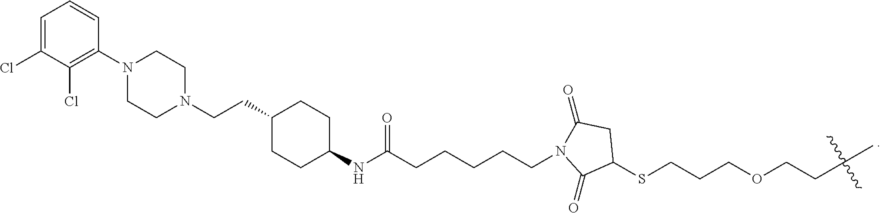 [SMILES: *CCOCCCSC1CC(=O)N(CCCCCC(=O)N[C@H]2CC[C@H](CCN3CCN(c4cccc(Cl)c4Cl)CC3)CC2)C1=O]

In certain embodiments, an additional chemical moiety is one described in WO 2012/030683. In certain embodiments, a provided ds oligonucleotide comprises a chemical structure (e.g., a linker, lipid, solubilizing group, and/or targeting ligand) described in WO 2012/030683.

In certain embodiments, a provided ds oligonucleotide comprises an additional chemical moiety and/or a modification (e.g., of nucleobase, sugar, internucleotidic linkage, etc.) described in: U.S. Pat. Nos. 5,688,941; 6,294,664; 6,320,017; 6,576,752; 5,258,506; 5,591,584; 4,958,013; 5,082,830; 5,118,802; 5,138,045; 6,783,931; 5,254,469; 5,414,077; 5,486,603; 5,112,963; 5,599,928; 6,900,297; 5,214,136; 5,109,124; 5,512,439; 4,667,025; 5,525,465; 5,514,785; 5,565,552; 5,541,313; 5,545,730; 4,835,263; 4,876,335; 5,578,717; 5,580,731; 5,451,463; 5,510,475; 4,904,582; 5,082,830; 4,762,779; 4,789,737; 4,824,941; 4,828,979; 5,595,726; 5,214,136; 5,245,022; 5,317,098; 5,371,241; 5,391,723; 4,948,882; 5,218,105; 5,112,963; 5,567,810; 5,574,142; 5,578,718; 5,608,046; 4,587,044; 4,605,735; 5,585,481; 5,292,873; 5,552,538; 5,512,667; 5,597,696; 5,599,923; 7,037,646; 5,587,371; 5,416,203; 5,262,536; 5,272,250; or 8,106,022.

In certain embodiments, an additional chemical moiety, e.g., a Mod, is connected via a linker. Various linkers are available in the art and may be utilized in accordance with the present disclosure, for example, those utilized for conjugation of various moieties with proteins (e.g., with antibodies to form antibody-drug conjugates), nucleic acids, etc. Certain useful linkers are described in U.S. Pat. No. 9,982,257, US 20170037399, US 20180216108, US 20180216107, U.S. Pat. No. 9,598,458, WO 2017/062862, WO 2018/067973, WO 2017/160741, WO 2017/192679, WO 2017/210647, WO 2018/098264, WO 2018/223056, WO 2018/237194, WO 2019/032607, WO 2019/055951, WO 2019/075357, WO 2019/200185, WO 2019/217784, and/or WO 2019/032612, the linker moieties of each which are independently incorporated herein by reference. In certain embodiments, a linker is, as non-limiting examples, L001, L004, L009 or L010. In certain embodiments, an oligonucleotide comprises a linker, but not an additional chemical moiety other than the linker. In certain embodiments, a ds oligonucleotide comprises a linker, but not an additional chemical moiety other than the linker, wherein the linker is L001, L004, L009, or L010.

As demonstrated herein, provided technologies can provide high levels of activities and/or desired properties, in certain embodiments, without utilizing particular structural elements (e.g., modifications, linkage configurations and/or patterns, etc.) reported to be desired and/or necessary (e.g., those reported in WO 2019/219581), though certain such structural elements may be incorporated into ds oligonucleotides in combination with various other structural elements in accordance with the present disclosure. For example, in certain embodiments, ds oligonucleotides of the present disclosure have fewer nucleosides 3′ to a nucleoside opposite to a target nucleoside (e.g., a target adenosine), contain one or more phosphorothioate internucleotidic linkages at one or more positions where a phosphorothioate internucleotidic linkage was reportedly not favored or not allowed, contain one or more Sp phosphorothioate internucleotidic linkages at one or more positions where a Sp phosphorothioate internucleotidic linkage was reportedly not favored or not allowed, contain one or more Rp phosphorothioate internucleotidic linkages at one or more positions where a Rp phosphorothioate internucleotidic linkage was reportedly not favored or not allowed, and/or contain different modifications (e.g., internucleotidic linkage modifications, sugar modifications, etc.) and/or stereochemistry at one or more locations compared to those reportedly favorable or required for certain oligonucleotide properties and/or activities (e.g., presence of 2′-MOE, absence of phosphorothioate linkages at certain positions, absence of Sp phosphorothioate linkages at certain positions, and/or absence of Rp phosphorothioate linkages at certain positions were reportedly favorable or required for certain oligonucleotide properties and/or activities; as demonstrated herein, provided technologies can provide desired properties and/or high activities without utilizing 2′-MOE, without avoiding phosphorothioate linkages at one or more such certain positions, without avoiding Sp phosphorothioate linkages at one or more such certain positions, and/or without avoiding Rp phosphorothioate linkages at one or more such certain positions). Additionally or alternatively, provided ds oligonucleotides incorporates structural elements that were not previously recognized such as utilization of certain modifications (e.g., base modifications, sugar modifications (e.g., 2′-F), linkage modifications (e.g., non-negatively charged internucleotidic linkages), additional moieties, etc.) and levels, patterns, and combinations thereof.

For example, in certain embodiments, as described herein, provided d oligonucleotides contain no more than 5, 6, 7, 8, 9, 10, 11 or 12 nucleosides 3′ to a nucleoside opposite to a target nucleoside (e.g., a target adenosine).

Alternatively or additionally, as described herein (e.g., illustrated in certain Examples), for structural elements 3′ to a nucleoside opposite to a target nucleoside (e.g., a target adenosine), in certain embodiments, about 50%-100% (e.g., about or at least about 50%, 55%, 60%, 65%, 70%, 75%, 80%, 85%, 90%, 95%) of internucleotidic linkages 3′ to a nucleoside opposite to a target nucleoside (e.g., a target adenosine) are each independently a modified internucleotidic linkage, which is optionally chirally controlled. In certain embodiments, no more than 1, 2, or 3 internucleotidic linkages 3′ to a nucleoside opposite to a target nucleoside are natural phosphate linkages. In certain embodiments, no such internucleotidic linkage is natural phosphate linkages. In certain embodiments, no more than 1 such internucleotidic linkage is natural phosphate linkages. In certain embodiments, no more than 2 such internucleotidic linkages are natural phosphate linkages. In certain embodiments, no more than 3 such internucleotidic linkages are natural phosphate linkages. In certain embodiments, each modified internucleotidic linkage is independently a phosphorothioate or a non-negatively charged internucleotidic linkage (e.g., n001). In certain embodiments, each phosphorothioate internucleotidic linkage is chirally controlled. In certain embodiments, no more than 1, 2, or 3 internucleotidic linkages 3′ to a nucleoside opposite to a target nucleoside are Rp phosphorothioate internucleotidic linkage.

Alternatively or additionally, as described herein (e.g., illustrated in certain Examples), in certain embodiments, about 50%-100% (e.g., about or at least about 50%, 55%, 60%, 65%, 70%, 75%, 80%, 85%, 90%, or 95%) of internucleotidic linkages 5′ to a nucleoside opposite to a target nucleoside (e.g., a target adenosine) are each independently a modified internucleotidic linkage, which is optionally chirally controlled. In certain embodiments, no or no more than 1, 2, or 3 internucleotidic linkages 5′ to a nucleoside opposite to a target nucleoside (e.g., a target adenosine) are not modified internucleotidic linkages. In certain embodiments, no or no more than 1, 2, or 3 internucleotidic linkages 5′ to a nucleoside opposite to a target nucleoside (e.g., a target adenosine) are not phosphorothioate internucleotidic linkages. In certain embodiments, no or no more than 1, 2, or 3 internucleotidic linkages 5′ to a nucleoside opposite to a target nucleoside (e.g., a target adenosine) are not Sp phosphorothioate internucleotidic linkages. In certain embodiments, no more than 1, 2, or 3 internucleotidic linkages 5′ to a nucleoside opposite to a target nucleoside (e.g., a target adenosine) are natural phosphate linkages. In certain embodiments, no such internucleotidic linkage is natural phosphate linkages. In certain embodiments, no more than 1 such internucleotidic linkage is natural phosphate linkages. In certain embodiments, no more than 2 such internucleotidic linkages are natural phosphate linkages. In certain embodiments, no more than 3 such internucleotidic linkages are natural phosphate linkages. In certain embodiments, each modified internucleotidic linkage is independently a phosphorothioate or a non-negatively charged internucleotidic linkage (e.g., n001). In certain embodiments, there are no 2, 3, or 4 consecutive internucleotidic linkages 5′ to a nucleoside opposite to a target nucleoside, each of which is not a phosphorothioate internucleotidic linkage. In certain embodiments, there are no 2, 3, or 4 consecutive internucleotidic linkages 5′ to a nucleoside opposite to a target nucleoside, each of which is chirally controlled and is not a Sp phosphorothioate internucleotidic linkage. In certain embodiments, no or no more than 1, 2, 3, 4, or 5 internucleotidic linkages 5′ to a nucleoside opposite to a target nucleoside (e.g., a target adenosine) are Rp phosphorothioate internucleotidic linkage. In certain embodiments, at least about 1, 2, 3, 4, 5, 6, 7, 8, 9, or 10, or about 1, 2, 3, 4, 5, 6, 7, 8, 9, 10, 11, 12, 13, 14, 15, 16, 18, 19, 20, 21, 22, 23, 24, 25, 26, 27, 28, 29, 30, 31, or 32, or about 50%-100% (e.g., about or at least about 50%, 55%, 60%, 65%, 70%, 75%, 80%, 85%, 90%, or 95%) of internucleotidic linkages 5′ to a nucleoside opposite to a target nucleoside (e.g., a target adenosine) are each independently chirally controlled and a Sp internucleotidic linkage. In certain embodiments, at least about 1, 2, 3, 4, 5, 6, 7, 8, 9, or 10, or about 1, 2, 3, 4, 5, 6, 7, 8, 9, 10, 11, 12, 13, 14, 15, 16, 18, 19, 20, 21, 22, 23, 24, 25, 26, 27, 28, 29, 30, 31, or 32, or about 50%-100% (e.g., about or at least about 50%, 55%, 60%, 65%, 70%, 75%, 80%, 85%, 90%, or 95%) of phosphorothioate internucleotidic linkages 5′ to a nucleoside opposite to a target nucleoside (e.g., a target adenosine) are each independently chirally controlled and are Sp. In certain embodiments, each phosphorothioate internucleotidic linkages 5′ to a nucleoside opposite to a target nucleoside (e.g., a target adenosine) is chirally controlled. In certain embodiments, each phosphorothioate internucleotidic linkages 5′ to a nucleoside opposite to a target nucleoside (e.g., a target adenosine) is Sp.

### Production of Oligonucleotides and Compositions

Various methods can be utilized for production of oligonucleotides and compositions and can be utilized in accordance with the present disclosure. For example, traditional phosphoramidite chemistry can be utilized to prepare stereorandom oligonucleotides and compositions, and certain reagents and chirally controlled technologies can be utilized to prepare chirally controlled oligonucleotide compositions, e.g., as described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, and/or WO 2020/191252, the reagents and methods of each of which is incorporated herein by reference.

In some embodiments, chirally controlled/stereoselective preparation of oligonucleotides and compositions thereof comprise utilization of a chiral auxiliary, e.g., as part of monomeric phosphoramidites. Examples of such chiral auxiliary reagents and phosphoramidites are described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, and/or WO 2020/191252, the chiral auxiliary reagents and phosphoramidites of each of which are independently incorporated herein by reference. In some embodiments, a chiral auxiliary is

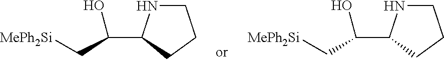

(DPSE chiral auxiliaries). In some embodiments, a chiral auxiliary is

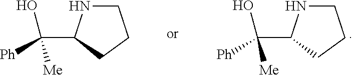 [SMILES: C[C@@](O)(c1ccccc1)[C@@H]1CCCN1 ; C[C@](O)(c1ccccc1)[C@H]1CCCN1]

In some embodiments, a chiral auxiliary is

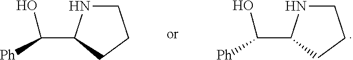 [SMILES: O[C@H](c1ccccc1)[C@@H]1CCCN1 ; O[C@@H](c1ccccc1)[C@H]1CCCN1]

In some embodiments, a chiral auxiliary comprises —SO₂R^{AU}, wherein R^{AU} is an optionally substituted group selected from C₁₋₂₀ aliphatic, C₁₋₂₀ heteroaliphatic having 1-10 heteroatoms, C₆₋₂₀ aryl, C₆₋₂₀ arylaliphatic, C₆₋₂₀ arylheteroaliphatic having 1-10 heteroatoms, 5-20 membered heteroaryl having 1-10 heteroatoms, and 3-20 membered heterocyclyl having 1-10 heteroatoms. In some embodiments, a chiral auxiliary is

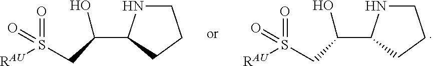

In some embodiments, R^{AU} is optionally substituted aryl. In some embodiments, R^{AU} is optionally substituted phenyl. In some embodiments, R^{AU} is optionally substituted C₁₋₆ aliphatic. In some embodiments, a chiral auxiliary is

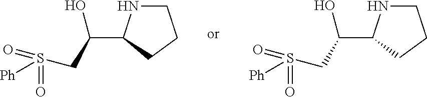 [SMILES: O=S(=O)(C[C@@H](O)[C@@H]1CCCN1)c1ccccc1 ; O=S(=O)(C[C@H](O)[C@H]1CCCN1)c1ccccc1]

(PSM chiral auxiliaries). In some embodiments, utilization of such chiral auxiliaries, e.g., preparation, phosphoramidites comprising such chiral auxiliaries, intermediate oligonucleotides comprising such auxiliaries (which auxiliaries are typically bonded to linkage phosphorus through —O— of —OH, and —NH— are optionally capped, e.g., by —C(O)R), protection, removal, etc., is described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, and/or WO 2020/191252 and incorporated herein by reference.

In some embodiments, chirally controlled preparation technologies, including oligonucleotide synthesis cycles, reagents and conditions are described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, and/or WO 2020/191252, the oligonucleotide synthesis methods, cycles, reagents and conditions of each of which are independently incorporated herein by reference.

Once synthesized, ds oligonucleotides targeting HSD17B13 and compositions are typically further purified. Suitable purification technologies are widely known and practiced by those skilled in the art, including but not limited to those described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, and/or WO 2020/191252, the purification technologies of each of which are independently incorporated herein by reference.

In some embodiments, a cycle comprises or consists of coupling, capping, modification and deblocking. In some embodiments, a cycle comprises or consists of coupling, capping, modification, capping and deblocking. These steps are typically performed in the order they are listed, but in some embodiments, as appreciated by those skilled in the art, the order of certain steps, e.g., capping and modification, may be altered. If desired, one or more steps may be repeated to improve conversion, yield and/or purity as those skilled in the art often perform in syntheses. For example, in some embodiments, coupling may be repeated; in some embodiments, modification (e.g., oxidation to install ═O, sulfurization to install ═S, etc.) may be repeated; in some embodiments, coupling is repeated after modification which can convert a P(III) linkage to a P(V) linkage which can be more stable under certain circumstances, and coupling is routinely followed by modification to convert newly formed P(III) linkages to P(V) linkages. In some embodiments, when steps are repeated, different conditions may be employed (e.g., concentration, temperature, reagent, time, etc.).

In some embodiments, oligonucleotides are linked to a solid support. In some embodiments, a solid support is a support for oligonucleotide synthesis. In some embodiments, a solid support comprises glass. In some embodiments, a solid support is CPG (controlled pore glass). In some embodiments, a solid support is polymer. In some embodiments, a solid support is polystyrene. In some embodiments, the solid support is Highly Crosslinked Polystyrene (HCP). In some embodiments, the solid support is hybrid support of Controlled Pore Glass (CPG) and Highly Cross-linked Polystyrene (HCP). In some embodiments, a solid support is a metal foam. In some embodiments, a solid support is a resin. In some embodiments, oligonucleotides are cleaved from a solid support.

Technologies for formulating provided oligonucleotides and/or preparing pharmaceutical compositions, e.g., for administration to subjects via various routes, are readily available in the art and can be utilized in accordance with the present disclosure, e.g., those described in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, and/or WO 2020/191252.

### Metabolites and Shortened Versions of Oligonucleotides

In some embodiments, a ds oligonucleotide targeting HSD17B13 corresponds to a metabolite produced by cleavage (e.g., enzymatic cleavage by a nuclease) of a longer oligonucleotide, e.g., a longer ds oligonucleotide targeting HSD17B13. In some embodiments, the present disclosure pertains to a ds oligonucleotide targeting HSD17B13 which corresponds to a portion, or fragment of a ds oligonucleotide targeting HSD17B13 disclosed herein.

In some embodiments, the present disclosure pertains to an oligonucleotide which corresponds to a metabolite of ads oligonucleotide targeting HSD17B13 disclosed herein. In some embodiments, the present disclosure pertains to an oligonucleotide which is 1, 2, 3, 4, 5, 6, 7, 8, 9, 10, 11, 12, 13, or more bases shorter than an oligonucleotide disclosed herein. In some embodiments, the present disclosure pertains to an oligonucleotide which has a base sequence which is 1, 2, 3, 4, 5, 6, 7, 8, 9, 10, 11, 12, 13, or more bases shorter than that of an oligonucleotide disclosed herein.

In some embodiments, a metabolite is designated as 3′-N— #, or 5′-N-#, wherein the # indicates the number of bases removed, and the 3′ or 5′ indicates which end of the molecule from which the bases were deleted. For example, 3′-N-1 indicates a fragment or metabolite wherein 1 base was removed from the 3′ end.

In some embodiments, the present disclosure perhaps to an oligonucleotide which corresponds to a fragment or metabolite of an oligonucleotide disclosed herein, wherein the fragment or metabolite can be described as corresponding to 3′-N-1, 3′-N-2, 3′-N-3, 3′-N-4, 3′-N-5, 3′-N-6, 3′-N-7, 3′-N-8, 3′-N-9, 3′-N-10, 3′-N-11, 3′-N-12, 5′-N-1, 5′-N-2, 5′-N-3, 5′-N4, 5′-N-5, 5′-N-6, 5′-N-7, 5′-N-8, 5′-N-9, 5′-N-10, 5′-N-11, or 5′-N-12 of an oligonucleotide described herein, wherein each T may be independently replaced with U and vice versa.

In some embodiments, the present disclosure pertains to an oligonucleotide which corresponds to a metabolite of an oligonucleotide, wherein the metabolite is truncated on the 5′ and/or 3′ end relative to the oligonucleotide disclosed herein, wherein each T may be independently replaced with U and vice versa. In some embodiments, the present disclosure pertains to an which corresponds to a metabolite of an oligonucleotide, wherein the metabolite is truncated on both the 5′ and 3′ end relative to the oligonucleotide disclosed herein, wherein each T may be independently replaced with U and vice versa. In some embodiments, the present disclosure pertains to an oligonucleotide which is 1, 2, 3, 4, 5, 6, 7, 8, 9, 10, 11, 12, 13, or more total bases shorter on the 5′ and/or 3′ end than an oligonucleotide disclosed herein. In some embodiments, the present disclosure pertains to an oligonucleotide which has a base sequence which is 1, 2, 3, 4, 5, 6, 7, 8, 9, 10, 11, 12, 13, or more bases total shorter on the 5′ and/or 3′ end than that of an oligonucleotide disclosed herein, wherein each T may be independently replaced with U and vice versa.

In some embodiments, the present disclosure pertains to an oligonucleotide which is a product of a cleavage of an oligonucleotide disclosed herein cleaved at a natural phosphate linkage. In some embodiments, the present disclosure pertains to an oligonucleotide which is a product of a cleavage of an oligonucleotide disclosed herein cleaved at a Rp phosphorothioate internucleotidic linkage.

Various technologies can be utilized to identify, characterize and/or assess metabolites and/or shortened ds oligonucleotides targeting HSD17B13 in accordance with the present disclosure, for example, those described in U.S. Ser. No. 10/479,995, US 2020/0056173, US 2018/0216107, US 2019/0127733, U.S. Ser. No. 10/450,568, US 2019/0249173, WO 2018/223056, WO 2018/223073, WO 2018/223081, WO 2019/032607, WO 2019/075357, WO 2019/200185, WO 2019/217784, WO 2019/032612, and/or WO 2020/191252.

### Characterization and Assessment

In some embodiments, properties and/or activities of ds oligonucleotides targeting HSD17B13 and compositions thereof can be characterized and/or assessed using various technologies available to those skilled in the art, e.g., biochemical assays (e.g., RNase H assays), cell based assays, animal models, clinical trials, etc.

In some embodiments, a method of identifying and/or characterizing an oligonucleotide composition, e.g., a ds oligonucleotide targeting HSD17B13 composition, comprises steps of:
providing at least one composition comprising a plurality of oligonucleotides; and
assessing delivery relative to a reference composition.

In some embodiments, the present disclosure provides a method of identifying and/or characterizing an oligonucleotide composition, e.g., a ds oligonucleotide targeting HSD17B13 composition, comprises steps of:
providing at least one composition comprising a plurality of oligonucleotides; and
assessing cellular uptake relative to a reference composition.

In some embodiments, the present disclosure provides a method of identifying and/or characterizing an oligonucleotide composition, e.g., a ds oligonucleotide targeting HSD17B13 composition, comprises steps of:
providing at least one composition comprising a plurality of oligonucleotides; and
assessing reduction of transcripts of a target gene and/or a product encoded thereby relative to a reference composition.

In some embodiments, the present disclosure provides a method of identifying and/or characterizing an oligonucleotide composition, e.g., a ds oligonucleotide targeting HSD17B13 composition, comprises steps of:
providing at least one composition comprising a plurality of oligonucleotides; and
assessing reduction of tau levels, its aggregation and/or spreading relative to a reference composition.

In some embodiments, properties and/or activities of oligonucleotides, e.g., ds oligonucleotides targeting HSD17B13, and compositions thereof are compared to reference oligonucleotides and compositions thereof, respectively.

In some embodiments, a reference oligonucleotide composition is a stereorandom oligonucleotide composition. In some embodiments, a reference oligonucleotide composition is a stereorandom composition of oligonucleotides of which all internucleotidic linkages are phosphorothioate. In some embodiments, a reference oligonucleotide composition is a DNA oligonucleotide composition with all phosphate linkages. In some embodiments, a reference oligonucleotide composition is otherwise identical to a provided chirally controlled oligonucleotide composition except that it is not chirally controlled. In some embodiments, a reference oligonucleotide composition is otherwise identical to a provided chirally controlled oligonucleotide composition except that it has a different pattern of stereochemistry. In some embodiments, a reference oligonucleotide composition is similar to a provided oligonucleotide composition except that it has a different modification of one or more sugar, base, and/or internucleotidic linkage, or pattern of modifications. In some embodiments, an oligonucleotide composition is stereorandom and a reference oligonucleotide composition is also stereorandom, but they differ in regards to sugar and/or base modification(s) or patterns thereof.

In some embodiments, a reference composition is a composition of oligonucleotides having the same base sequence and the same chemical modifications. In some embodiments, a reference composition is a composition of oligonucleotides having the same base sequence and the same pattern of chemical modifications. In some embodiments, a reference composition is a non-chirally controlled (or stereorandom) composition of oligonucleotides having the same base sequence and chemical modifications. In some embodiments, a reference composition is a non-chirally controlled (or stereorandom) composition of oligonucleotides of the same constitution but is otherwise identical to a provided chirally controlled oligonucleotide composition.

In some embodiments, a reference oligonucleotide composition is of oligonucleotides having a different base sequence. In some embodiments, a reference oligonucleotide composition is of oligonucleotides that do not target HSD17B13 (e.g., as negative control for certain assays).

In some embodiments, a reference composition is a composition of oligonucleotides having the same base sequence but different chemical modifications, including but not limited to chemical modifications described herein. In some embodiments, a reference composition is a composition of oligonucleotides having the same base sequence but different patterns of internucleotidic linkages and/or stereochemistry of internucleotidic linkages and/or chemical modifications.

Various methods are known in the art for detection of gene products, the expression, level and/or activity of which may be altered after introduction or administration of a provided oligonucleotide. For example, transcripts and their knockdown can be detected and quantified with qPCR, and protein levels can be determined via Western blot.

In some embodiments, assessment of efficacy of oligonucleotides can be performed in biochemical assays or in vitro in cells. In some embodiments, ds oligonucleotides targeting HSD17B13 can be introduced to cells via various methods available to those skilled in the art, e.g., gymnotic delivery, transfection, lipofection, etc.

In some embodiments, the efficacy of a putative ds oligonucleotide targeting HSD17B13 can be tested in vitro.

In some embodiments, the efficacy of a putative ds oligonucleotide targeting HSD17B13 can be tested in vitro using any known method of testing the expression, level and/or activity of a HSD17B13 gene or gene product thereof.

In some embodiments, an animal model administered a ds oligonucleotide targeting HSD17B13 can be evaluated for safety and/or efficacy.

In some embodiments, the effect(s) of administration of an oligonucleotide to an animal can be evaluated, including any effects on behavior, inflammation, and toxicity. In some embodiments, following dosing, animals can be observed for signs of toxicity including trouble grooming, lack of food consumption, and any other signs of lethargy.

In some embodiments, following administration of a ds oligonucleotide targeting HSD17B13 to an animal, the animal can be sacrificed and analysis of tissues or cells can be performed to determine changes in HSD17B13, or other biochemical or other changes. In some embodiments, following necropsy, liver, heart, lung, kidney, and spleen can be collected, fixed, and processed for histopathological evaluation (standard light microscopic examination of hematoxylin and eosin-stained tissue slides).

In some embodiments, following administration of a ds oligonucleotide targeting HSD17B13 to an animal, behavioral changes can be monitored or assessed. In some embodiments, such an assessment can be performed using a technique described in the scientific literature.

Various effects of testing in animals described herein can also be monitored in human subjects or patients following administration of a ds oligonucleotide targeting HSD17B13. In addition, the efficacy of a ds oligonucleotide targeting HSD17B13 in a human subject can be measured by evaluating, after administration of the oligonucleotide, any of various parameters known in the art, including but not limited to a reduction in a symptom of ****.

In some embodiments, following human treatment with an oligonucleotide, or contacting a cell or tissue in vitro with an oligonucleotide, cells and/or tissues are collected for analysis.

In some embodiments, in various cells and/or tissues, target nucleic acid levels can be quantitated by methods available in the art, many of which can be accomplished with commercially available kits and materials. Such methods include, e.g., Northern blot analysis, competitive polymerase chain reaction (PCR), quantitative real-time PCR, etc. RNA analysis can be performed on total cellular RNA or poly(A)+ mRNA. Probes and primers are designed to hybridize to a nucleic acid to be detected. Methods for designing real-time PCR probes and primers are well known and widely practiced in the art. For example, to detect and quantify HSD17B13 RNA, an example method comprises isolation of total RNA (e.g., including mRNA) from a cell or animal treated with an oligonucleotide or a composition and subjecting the RNA to reverse transcription and/or quantitative real-time PCR, for example, as described herein, or in: Moon et al. 2012 Cell Metab. 15: 240-246.

In some embodiments, protein levels can be evaluated or quantitated in various methods known in the art, e.g., enzyme-linked immunosorbent assay (ELISA), Western blot analysis (immunoblotting), immunocytochemistry, fluorescence-activated cell sorting (FACS), immunohistochemistry, immunoprecipitation, protein activity assays (for example, caspase activity assays), and quantitative protein assays. Antibodies useful for the detection of mouse, rat, monkey, and human proteins are commercially available or can be generated if needed. For example, various HSD17B13 antibodies have been reported.

Various technologies are available and/or known in the art for detecting levels of oligonucleotides or other nucleic acids. Such technologies are useful for detecting ds oligonucleotides targeting HSD17B13 when administered to assess, e.g., delivery, cell uptake, stability, distribution, etc.

In some embodiments, selection criteria are used to evaluate the data resulting from various assays and to select particularly desirable oligonucleotides, e.g., desirable ds oligonucleotides targeting HSD17B13, with certain properties and activities. In some embodiments, selection criteria include an IC₅₀ of less than about 10 nM, less than about 5 nM or less than about 1 nM. In some embodiments, selection criteria for a stability assay include at least 50% stability [at least 50% of an oligonucleotide is still remaining and/or detectable] at Day 1. In some embodiments, selection criteria for a stability assay include at least 50% stability at Day 2. In some embodiments, selection criteria for a stability assay include at least 50% stability at Day 3. In some embodiments, selection criteria for a stability assay include at least 50% stability at Day 4. In some embodiments, selection criteria for a stability assay include at least 50% stability at Day 5. In some embodiments, selection criteria for a stability assay include at least 80% [at least 80% of the oligonucleotide remains] at Day 5. In some embodiments, efficacy of a ds oligonucleotide targeting HSD17B13 is assessed directly or indirectly by monitoring, measuring or detecting a change in a condition, disorder, or disease or a biological pathway associated with HSD17B13.

In some embodiments, efficacy of a ds oligonucleotide targeting HSD17B13 is assessed directly or indirectly by monitoring, measuring or detecting a change in a response to be affected by HSD17B13 knockdown.

In some embodiments, a provided oligonucleotide (e.g., a ds oligonucleotide targeting HSD17B13) can by analyzed by a sequence analysis to determine what other genes [e.g., genes which are not a target gene (e.g., HSD17B13)] have a sequence which is complementary to the base sequence of the provided oligonucleotide (e.g., the ds oligonucleotide targeting HSD17B13) or which have 0, 1, 2 or more mismatches from the base sequence of the provided oligonucleotide (e.g., the ds oligonucleotide targeting HSD17B13). Knockdown, if any, by the oligonucleotide of these potential off-targets can be determined to evaluate potential off-target effects of an oligonucleotide (e.g., a ds oligonucleotide targeting HSD17B13). In some embodiments, an off-target effect is also termed an unintended effect and/or related to hybridization to a bystander (non-target) sequence or gene.

Oligonucleotides which have been evaluated and tested for efficacy in knocking down HSD17B13 have various uses, e.g., in treatment or prevention of a HSD17B13-associated condition, disorder, or disease or a symptom thereof.

In some embodiments, a ds oligonucleotide targeting HSD17B13 which has been evaluated and tested for its ability to provide a particular biological effect (e.g., reduction of level, expression and/or activity of a HSD17B13 target gene or a gene product thereof) can be used to treat, ameliorate and/or prevent a HSD17B13-associated condition, disorder, or disease.

### Treatment of HSD17B13-Associated Conditions, Disorders, or Diseases

In some embodiments, the present disclosure provides a ds oligonucleotide targeting HSD17B13, which targets HSD17B13 and directs target-specific knockdown of HSD17B13. In some embodiments, the present disclosure provides methods for preventing and/or treating HSD17B13-associated conditions, disorders, or diseases using provided ds oligonucleotides targeting HSD17B13 and compositions thereof. In some embodiments, the present disclosure provides oligonucleotides and compositions thereof for use as medicaments, e.g., for HSD17B13-associated conditions, disorders, or diseases. In some embodiments, the present disclosure provides oligonucleotides and compositions thereof for use in the treatment of HSD17B13-associated conditions, disorders, or diseases. In some embodiments, the present disclosure provides oligonucleotides and compositions thereof for the manufacture of medicaments for the treatment of HSD17B13-associated conditions, disorders, or diseases.

In some embodiments, the present disclosure provides a method for treating and/or ameliorating one or more symptoms associated with a HSD17B13-associated condition, disorder, or disease in a mammal in need thereof, the method comprising administering to the mammal a therapeutically effective amount of a ds oligonucleotide targeting HSD17B13 or a composition thereof. In some embodiments, the present disclosure provides a method for reducing susceptibility to a HSD17B13-associated condition, disorder, or disease in a mammal in need thereof, the method comprising: administering to the mammal a therapeutically effective amount of a ds oligonucleotide targeting HSD17B13 or a composition thereof. In some embodiments, the present disclosure provides a method for preventing or delaying the onset of a HSD17B13-associated condition, disorder, or disease in a mammal in need thereof, the method comprising: administering to the mammal a therapeutically effective amount of a ds oligonucleotide targeting HSD17B13 or a composition thereof. In some embodiments, the present disclosure provides a method for treating and/or ameliorating one or more symptoms associated with a HSD17B13-associated condition, disorder, or disease in a mammal in need thereof, the method comprising: administering to the mammal a therapeutically effective amount of a nucleic acid-lipid particle comprising a ds oligonucleotide targeting HSD17B13. In some embodiments, the present disclosure provides a method for reducing susceptibility to a HSD17B13-associated condition, disorder, or disease in a mammal in need thereof, the method comprising: administering to the mammal a therapeutically effective amount of a nucleic acid-lipid particle comprising a ds oligonucleotide targeting HSD17B13. In some embodiments, the present disclosure provides a method for preventing or delaying the onset of a HSD17B13-associated condition, disorder, or disease in a mammal in need thereof, the method comprising: administering to the mammal a therapeutically effective amount of a nucleic acid-lipid particle comprising a ds oligonucleotide targeting HSD17B13. In some embodiments, a mammal is a human. In some embodiments, a mammal is susceptible to, afflicted with and/or suffering from a HSD17B13-associated condition, disorder, or disease. In some embodiments, a HSD17B13-associated condition, disorder, or disease is NAFLD, NASH, ASH, alcoholic liver disease, nonalcoholic liver disease, alcoholic cirrhosis, nonalcoholic cirrhosis, steatohepatitis, hepatic steatosis, hepatoceullar carcinoma, HCV hepatitis, chronic hepatitis, hereditary hemochromatosis, primary sclerosing cholangitis, drug induced liver injury, or hepatocellular necrosis.

In some embodiments, provided oligonucleotides and compositions are useful for preventing and/or treating neurodegenerative diseases, e.g., NAFLD, NASH, ASH, alcoholic liver disease, nonalcoholic liver disease, alcoholic cirrhosis, nonalcoholic cirrhosis, steatohepatitis, hepatic steatosis, hepatoceullar carcinoma, HCV hepatitis, chronic hepatitis, hereditary hemochromatosis, primary sclerosing cholangitis, drug induced liver injury, or hepatocellular necrosis, etc. In some embodiments, the present disclosure provides methods for preventing and/or treating a neurodegenerative disease, e.g., NAFLD, NASH, ASH, alcoholic liver disease, nonalcoholic liver disease, alcoholic cirrhosis, nonalcoholic cirrhosis, steatohepatitis, hepatic steatosis, hepatoceullar carcinoma, HCV hepatitis, chronic hepatitis, hereditary hemochromatosis, primary sclerosing cholangitis, drug induced liver injury, or hepatocellular necrosis, etc., comprising administering to a subject susceptible to or suffering therefrom a therapeutically effective amount of a ds oligonucleotide targeting HSD17B13 or a composition as described herein. In some embodiments, the present disclosure provides methods for treating a neurodegenerative disease comprising administering to a subject suffering therefrom a therapeutically effective amount of a ds oligonucleotide targeting HSD17B13 or a composition as described herein. In some embodiments, the present disclosure provides methods for preventing and/or treating a tauopathy comprising administering to a subject susceptible to or suffering therefrom a therapeutically effective amount of a ds oligonucleotide targeting HSD17B13 or a composition as described herein. In some embodiments, the present disclosure provides methods for treating a tauopathy comprising administering to a subject suffering therefrom a therapeutically effective amount of a ds oligonucleotide targeting HSD17B13 or a composition as described herein. In some embodiments, the present disclosure provides methods for preventing and/or treating NAFLD, NASH, ASH, alcoholic liver disease, nonalcoholic liver disease, alcoholic cirrhosis, nonalcoholic cirrhosis, steatohepatitis, hepatic steatosis, hepatoceullar carcinoma, HCV hepatitis, chronic hepatitis, hereditary hemochromatosis, primary sclerosing cholangitis, drug induced liver injury, or hepatocellular necrosis comprising administering to a subject susceptible to or suffering therefrom a therapeutically effective amount of a ds oligonucleotide targeting HSD17B13 or a composition as described herein. In some embodiments, the present disclosure provides methods for treating NAFLD, NASH, ASH, alcoholic liver disease, nonalcoholic liver disease, alcoholic cirrhosis, nonalcoholic cirrhosis, steatohepatitis, hepatic steatosis, hepatoceullar carcinoma, HCV hepatitis, chronic hepatitis, hereditary hemochromatosis, primary sclerosing cholangitis, drug induced liver injury, or hepatocellular necrosis comprising administering to a subject suffering therefrom a therapeutically effective amount of ads oligonucleotide targeting HSD17B13 or a composition as described herein. In some embodiments, a subject has increased HSD17B13 expression, and/or increased levels of one or more HSD17B13 products (e.g., transcripts, proteins, etc.) compared to, e.g., a health subject (or a population thereof), a subject that is not susceptible to and/or not suffering from an HSD17B13-associated disease (or a population thereof), etc.

In some embodiments, provided oligonucleotides and compositions may be optionally utilized in combination with one or more other therapeutic agents.

### Administration of Oligonucleotides and Compositions

Many delivery methods, regimen, etc. can be utilized in accordance with the present disclosure for administering provided ds oligonucleotides and compositions thereof (typically pharmaceutical compositions for therapeutic purposes), including various technologies known in the art.

In some embodiments, an oligonucleotide composition, e.g., a ds oligonucleotide targeting HSD17B13 composition, is administered at a dose and/or frequency lower than that of an otherwise comparable reference oligonucleotide composition and has comparable or improved effects. In some embodiments, a chirally controlled oligonucleotide composition is administered at a dose and/or frequency lower than that of a comparable, otherwise identical stereorandom reference oligonucleotide composition and with comparable or improved effects, e.g., in improving the knockdown of the target transcript.

In some embodiments, the present disclosure recognizes that properties and activities, e.g., knockdown activity, stability, toxicity, etc. of oligonucleotides and compositions thereof can be modulated and optimized by chemical modifications and/or stereochemistry. In some embodiments, the present disclosure provides methods for optimizing oligonucleotide properties and/or activities through chemical modifications and/or stereochemistry. In some embodiments, the present disclosure provides oligonucleotides and compositions thereof with improved properties and/or activities. Without wishing to be bound by any theory, due to, e.g., their better activity, stability, delivery, distribution, toxicity, pharmacokinetic, pharmacodynamics and/or efficacy profiles, Applicant notes that provided oligonucleotides and compositions thereof in some embodiments can be administered at lower dosage and/or reduced frequency to achieve comparable or better efficacy, and in some embodiments can be administered at higher dosage and/or increased frequency to provide enhanced effects.

In some embodiments, the present disclosure provides, in a method of administering an oligonucleotide composition comprising a plurality of oligonucleotides sharing a common base sequence, the improvement comprising administering an oligonucleotide comprising a plurality of oligonucleotides that is characterized by improved delivery relative to a reference oligonucleotide composition of the same common base sequence.

In some embodiments, provided oligonucleotides, compositions and methods provide improved delivery. In some embodiments, provided oligonucleotides, compositions and methods provide improved cytoplasmatic delivery. In some embodiments, improved delivery is to a population of cells. In some embodiments, improved delivery is to a tissue. In some embodiments, improved delivery is to an organ. In some embodiments, improved delivery is to an organism, e.g., a patient or subject. Example structural elements (e.g., chemical modifications, stereochemistry, combinations thereof, etc.), oligonucleotides, compositions and methods that provide improved delivery are extensively described in the present disclosure.

Various dosing regimens can be utilized to administer oligonucleotides and compositions of the present disclosure. In some embodiments, multiple unit doses are administered, separated by periods of time. In some embodiments, a given composition has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second (or subsequent) dose amount that is the same as or different from the first dose (or another prior dose) amount. In some embodiments, a chirally controlled oligonucleotide composition is administered according to a dosing regimen that differs from that utilized for a non-chirally controlled (e.g., stereorandom) oligonucleotide composition of the same sequence, and/or of a different chirally controlled oligonucleotide composition of the same sequence. In some embodiments, a chirally controlled oligonucleotide composition is administered according to a dosing regimen that is reduced as compared with that of a chirally uncontrolled (e.g., stereorandom) oligonucleotide composition of the same sequence in that it achieves a lower level of total exposure over a given unit of time, involves one or more lower unit doses, and/or includes a smaller number of doses over a given unit of time. In some embodiments, a chirally uncontrolled oligonucleotide is administered according to a dosing regimen that extends for a longer period of time than does that of a chirally uncontrolled (e.g., stereorandom) oligonucleotide composition of the same sequence Without wishing to be limited by theory, Applicant notes that in some embodiments, the shorter dosing regimen, and/or longer time periods between doses, may be due to the improved stability, bioavailability, and/or efficacy of a chirally controlled oligonucleotide composition. In some embodiments, with their improved delivery (and other properties), provided compositions can be administered in lower dosages and/or with lower frequency to achieve biological effects, for example, clinical efficacy.

### Pharmaceutical Compositions

When used as therapeutics, a provided ds oligonucleotide, e.g., a ds oligonucleotide targeting HSD17B13, or oligonucleotide composition thereof is typically administered as a pharmaceutical composition. In some embodiments, the present disclosure provides pharmaceutical compositions comprising a provided compound, e.g., an oligonucleotide, or a pharmaceutically acceptable salt thereof, and a pharmaceutical carrier. In some embodiments, for therapeutic and clinical purposes, oligonucleotides of the present disclosure are provided as pharmaceutical compositions. As appreciated by those skilled in the art, oligonucleotides of the present disclosure can be provided in their acid, base or salt forms. In some embodiments, oligonucleotides can be in acid forms, e.g., for natural phosphate linkages, in the form of —OP(O)(OH)O—; for phosphorothioate internucleotidic linkages, in the form of —OP(O)(SH)O—; etc. In some embodiments, ds oligonucleotides targeting HSD17B13 can be in salt forms, e.g., for natural phosphate linkages, in the form of —OP(O)(ONa)O— in sodium salts; for phosphorothioate internucleotidic linkages, in the form of —OP(O)(SNa)O— in sodium salts; etc. Unless otherwise noted, oligonucleotides of the present disclosure can exist in acid, base and/or salt forms.

In some embodiments, a pharmaceutical composition is a liquid composition. In some embodiments, a pharmaceutical composition is provided by dissolving a solid oligonucleotide composition, or diluting a concentrated oligonucleotide composition, using a suitable solvent, e.g., water or a pharmaceutically acceptable buffer. In some embodiments, liquid compositions comprise anionic forms of provided oligonucleotides and one or more cations. In some embodiments, liquid compositions have pH values in the weak acidic, about neutral, or basic range. In some embodiments, pH of a liquid composition is about a physiological pH, e.g., about 7.4.

In some embodiments, a provided oligonucleotide is formulated for administration to and/or contact with a body cell and/or tissue expressing its target. For example, in some embodiments, a provided ds oligonucleotide targeting HSD17B13 is formulated for administration to a body cell and/or tissue expressing HSD17B13. In some embodiments, such a body cell and/or tissue are a neuron or a cell and/or tissue of the central nervous system. In some embodiments, broad distribution of oligonucleotides and compositions may be achieved with intraparenchymal administration, intrathecal administration, or intracerebroventricular administration.

In some embodiments, the pharmaceutical composition is formulated for intravenous injection, oral administration, buccal administration, inhalation, nasal administration, topical administration, ophthalmic administration or otic administration. In some embodiments, the pharmaceutical composition is a tablet, a pill, a capsule, a liquid, an inhalant, a nasal spray solution, a suppository, a suspension, a gel, a colloid, a dispersion, a suspension, a solution, an emulsion, an ointment, a lotion, an eye drop or an ear drop.

In some embodiments, the present disclosure provides a pharmaceutical composition comprising chirally controlled oligonucleotide or composition thereof, in admixture with a pharmaceutically acceptable inactive ingredient (e.g., a pharmaceutically acceptable excipient, a pharmaceutically acceptable carrier, etc.). In some embodiments, the present disclosure provides a pharmaceutical composition delivering chirally controlled oligonucleotide or composition thereof, in admixture with a pharmaceutically acceptable inactive ingredient (e.g., a pharmaceutically acceptable excipient, a pharmaceutically acceptable carrier, etc.). One of skill in the art will recognize that the pharmaceutical compositions include pharmaceutically acceptable salts of provided oligonucleotide or compositions. In some embodiments, a pharmaceutical composition is a chirally controlled oligonucleotide composition. In some embodiments, a pharmaceutical composition is a stereopure oligonucleotide composition.

In some embodiments, the present disclosure provides salts of oligonucleotides and pharmaceutical compositions thereof. In some embodiments, a salt is a pharmaceutically acceptable salt. In some embodiments, a pharmaceutical composition comprises an oligonucleotide, optionally in its salt form, and a sodium salt. In some embodiments, a pharmaceutical composition comprises an oligonucleotide, optionally in its salt form, and sodium chloride. In some embodiments, each hydrogen ion of an oligonucleotide that may be donated to a base (e.g., under conditions of an aqueous solution, a pharmaceutical composition, etc.) is replaced by a non-H⁺ cation. For example, in some embodiments, a pharmaceutically acceptable salt of an oligonucleotide is an all-metal ion salt, wherein each hydrogen ion (for example, of —OH, —SH, etc.) of each internucleotidic linkage (e.g., a natural phosphate linkage, a phosphorothioate internucleotidic linkage, etc.) is replaced by a metal ion. Various suitable metal salts for pharmaceutical compositions are widely known in the art and can be utilized in accordance with the present disclosure. In some embodiments, a pharmaceutically acceptable salt is a sodium salt. In some embodiments, a pharmaceutically acceptable salt is magnesium salt. In some embodiments, a pharmaceutically acceptable salt is a calcium salt. In some embodiments, a pharmaceutically acceptable salt is a potassium salt. In some embodiments, a pharmaceutically acceptable salt is an ammonium salt (cation N(R)₄⁺). In some embodiments, a pharmaceutically acceptable salt comprises one and no more than one types of cation. In some embodiments, a pharmaceutically acceptable salt comprises two or more types of cation. In some embodiments, a cation is Li⁺, Na⁺, K⁺, Mg²⁺ or Ca²⁺. In some embodiments, a pharmaceutically acceptable salt is an all-sodium salt. In some embodiments, a pharmaceutically acceptable salt is an all-sodium salt, wherein each internucleotidic linkage which is a natural phosphate linkage (acid form —O—P(O)(OH)—O—), if any, exists as its sodium salt form (—O—P(O)(ONa)—O—), and each internucleotidic linkage which is a phosphorothioate internucleotidic linkage (acid form —O—P(O)(SH)—O—), if any, exists as its sodium salt form (—O—P(O)(SNa)—O—).

Various technologies for delivering nucleic acids and/or oligonucleotides are known in the art can be utilized in accordance with the present disclosure. For example, a variety of supramolecular nanocarriers can be used to deliver nucleic acids. Example nanocarriers include, but are not limited to liposomes, cationic polymer complexes and various polymeric compounds. Complexation of nucleic acids with various polycations is another approach for intracellular delivery; this includes use of PEGylated polycations, polyethyleneamine (PEI) complexes, cationic block co-polymers, and dendrimers. Several cationic nanocarriers, including PEI and polyamidoamine dendrimers help to release contents from endosomes. Other approaches include use of polymeric nanoparticles, microspheres, liposomes, dendrimers, biodegradable polymers, conjugates, prodrugs, inorganic colloids such as sulfur or iron, antibodies, implants, biodegradable implants, biodegradable microspheres, osmotically controlled implants, lipid nanoparticles, emulsions, oily solutions, aqueous solutions, biodegradable polymers, poly(lactide-coglycolic acid), poly(lactic acid), liquid depot, polymer micelles, quantum dots and lipoplexes. In some embodiments, an oligonucleotide is conjugated to another molecule.

In therapeutic and/or diagnostic applications, compounds, e.g., oligonucleotides, of the disclosure can be formulated for a variety of modes of administration, including systemic and topical or localized administration. Techniques and formulations generally may be found in Remington, The Science and Practice of Pharmacy (20th ed. 2000).

Pharmaceutically acceptable salts for basic moieties are generally well known to those of ordinary skill in the art, and may include, e.g., acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington, The Science and Practice of Pharmacy (20th ed. 2000). Preferred pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

In some embodiments, ds oligonucleotides targeting HSD17B13 are formulated in pharmaceutical compositions described in WO 2005/060697, WO 2011/076807 or WO 2014/136086.

Depending on the specific conditions, disorders, or diseases being treated, provided agents, e.g., oligonucleotides, may be formulated into liquid or solid dosage forms and administered systemically or locally. Provided oligonucleotides may be delivered, for example, in a timed- or sustained-low release form as is known to those skilled in the art. Techniques for formulation and administration may be found in Remington, The Science and Practice of Pharmacy (20th ed. 2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections or another mode of delivery.

For injection, provided agents, e.g., oligonucleotides may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulations. Such penetrants are generally known in the art and can be utilized in accordance with the present disclosure.

Use of pharmaceutically acceptable carriers to formulate compounds, e.g., provided oligonucleotides, for the practice of the disclosure into dosages suitable for various mods of administration is well known in the art. With proper choice of carrier and suitable manufacturing practice, compositions of the present disclosure, e.g., those formulated as solutions, may be administered via various routes, e.g., parenterally, such as by intravenous injection.

In some embodiments, a composition comprising a ds oligonucleotide targeting HSD17B13 further comprises any or all of: calcium chloride dihydrate, magnesium chloride hexahydrate, potassium chloride, sodium chloride, sodium phosphate dibasic anhydrous, sodium phosphate, monobasic dihydrate, and/or water for Injection. In some embodiments, a composition further comprises any or all of: calcium chloride dihydrate (0.21 mg) USP, magnesium chloride hexahydrate (0.16 mg) USP, potassium chloride (0.22 mg) USP, sodium chloride (8.77 mg) USP, sodium phosphate dibasic anhydrous (0.10 mg) USP, sodium phosphate monobasic dihydrate (0.05 m g) USP, and Water for Injection USP.

In some embodiments, a composition comprising an oligonucleotide further comprises any or all of: cholesterol, (6Z,9Z,28Z,31Z)-heptatriaconta-6,9,28,31-tetraen-19-yl-4-(dimethylamino) butanoate(DLin-MC3-DMA), 1,2-distearoyl-sn-glycero-3-phosphocholine (DSPC), alpha-(3′-{[1,2-di(myristyloxy)propanoxy] carbonylamino}propyl)-omega-methoxy, polyoxyethylene(PEG2000-C-DMG), potassium phosphate monobasic anhydrous NF, sodium chloride, sodium phosphate dibasic heptahydrate, and Water for Injection. In some embodiments, the pH of a composition comprising a ds oligonucleotide targeting HSD17B13 is -7.0. In some embodiments, a composition comprising an oligonucleotide further comprises any or all of: 6.2 mg cholesterol USP, 13.0 mg (6Z,9Z,28Z,31Z)-heptatriaconta-6,9,28,31-tetraen-19-yl-4-(dimethylamino) butanoate(DLin-MC3-DMA), 3.3 mg 1,2-distearoyl-sn-glycero-3-phosphocholine (DSPC), 1.6 mg α-(3′-{[1,2-di(myristyloxy)propanoxy]carbonylamino}propyl)-ω-methoxy, polyoxyethylene(PEG2000-C-DMG), 0.2 mg potassium phosphate monobasic anhydrous NF, 8.8 mg sodium chloride USP, 2.3 mg sodium phosphate dibasic heptahydrate USP, and Water for Injection USP, in an approximately 1 mL total volume.

Provided compounds, e.g., oligonucleotides, can be formulated readily using pharmaceutically acceptable carriers well known in the art into dosages suitable for oral administration. In some embodiments, such carriers enable provided oligonucleotides to be formulated as tablets, pills, capsules, liquids, gels, syrups, slurries, suspensions and the like, for, e.g., oral ingestion by a subject (e.g., patient) to be treated.

For nasal or inhalation delivery, provided compounds, e.g., oligonucleotides, may be formulated by methods known to those of skill in the art, and may include, e.g., examples of solubilizing, diluting, or dispersing substances such as saline, preservatives, such as benzyl alcohol, absorption promoters, and fluorocarbons.

In certain embodiments, parenteral administration is by injection, by, e.g., a syringe, a pump, etc. In certain embodiments, an injection is a bolus injection. In certain embodiments, an injection is administered directly to a tissue or location, such as striatum, caudate, cortex, hippocampus and/or cerebellum.

In certain embodiments, methods of specifically localizing provided compounds, e.g., oligonucleotides, such as by bolus injection, may decrease median effective concentration (EC50) by a factor of 20, 25, 30, 35, 40, 45 or 50. In certain embodiments, a targeted tissue is brain tissue. In certain embodiments, a targeted tissue is striatal tissue. In certain embodiments, decreasing EC50 is desirable because it reduces the dose required to achieve a pharmacological result in a patient in need thereof.

In certain embodiments, a provided oligonucleotide is delivered by injection or infusion once every month, every two months, every 90 days, every 3 months, every 6 months, twice a year or once a year.

Pharmaceutical compositions suitable for use in the present disclosure include compositions wherein the active ingredients, e.g., oligonucleotides, are contained in effective amounts to achieve their intended purposes. Determination of the effective amounts is well within the capability of those skilled in the art, especially in light of the detailed disclosure provided herein.

In addition to active ingredients, pharmaceutical compositions may contain suitable pharmaceutically acceptable carriers comprising excipients and auxiliaries which facilitate processing of an active compound into preparations which can be used pharmaceutically. Preparations formulated for oral administration may be in the form of tablets, dragees, capsules, or solutions.

In some embodiments, pharmaceutical compositions for oral use can be obtained by combining an active compound with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

In some embodiments, dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. Push-fit capsules can contain active ingredients, e.g., oligonucleotides, in admixture with fillers such as lactose, binders such as starches, and/or lubricants such as talc or magnesium stearate and, optionally, stabilizers. In soft capsules, active compounds, e.g., oligonucleotides, may be dissolved or suspended in suitable liquids, such as fatty oils, liquid paraffin, or liquid polyethylene glycols (PEGs). In addition, stabilizers may be added.

In some embodiments, a provided composition comprises a lipid. In some embodiments, a lipid is conjugated to an active compound, e.g., an oligonucleotide. In some embodiments, a lipid is not conjugated to an active compound. In some embodiments, a lipid comprises a C₁₀-C₄₀ linear, saturated or partially unsaturated, aliphatic chain. In some embodiments, a lipid comprises a C₁₀-C₄₀ linear, saturated or partially unsaturated, aliphatic chain, optionally substituted with one or more C₁₋₄ aliphatic group. In some embodiments, the lipid is selected from the group consisting of lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl alcohol. In some embodiments, an active compound is a provided oligonucleotide. In some embodiments, a composition comprises a lipid and an active compound, and further comprises another component which is another lipid or a targeting compound or moiety. In some embodiments, a lipid is an amino lipid; an amphipathic lipid; an anionic lipid; an apolipoprotein; a cationic lipid; a low molecular weight cationic lipid; a cationic lipid such as CLinDMA and DLinDMA; an ionizable cationic lipid; a cloaking component; a helper lipid; a lipopeptide; a neutral lipid; a neutral zwitterionic lipid; a hydrophobic small molecule; a hydrophobic vitamin; a PEG-lipid; an uncharged lipid modified with one or more hydrophilic polymers; phospholipid; a phospholipid such as 1,2-dioleoyl-sn-glycero-3-phosphoethanolamine; a stealth lipid; a sterol; a cholesterol; a targeting lipid; or another lipid described herein or reported in the art suitable for pharmaceutical uses. In some embodiments, a composition comprises a lipid and a portion of another lipid capable of mediating at least one function of another lipid. In some embodiments, a targeting compound or moiety is capable of targeting a compound (e.g., an oligonucleotide) to a particular cell or tissue or subset of cells or tissues. In some embodiments, a targeting moiety is designed to take advantage of cell- or tissue-specific expression of particular targets, receptors, proteins, or another subcellular component. In some embodiments, a targeting moiety is a ligand (e.g., a small molecule, antibody, peptide, protein, carbohydrate, aptamer, etc.) that targets a composition to a cell or tissue, and/or binds to a target, receptor, protein, or another subcellular component.

Certain example lipids for delivery of an active compound, e.g., an oligonucleotide, allow (e.g., do not prevent or interfere with) the function of an active compound. In some embodiments, a lipid is lauric acid, myristic acid, palmitic acid, stearic acid, oleic acid, linoleic acid, alpha-linolenic acid, gamma-linolenic acid, docosahexaenoic acid (cis-DHA), turbinaric acid or dilinoleyl alcohol.

As described in the present disclosure, lipid conjugation, such as conjugation with fatty acids, may improve one or more properties of oligonucleotides.

In some embodiments, a composition for delivery of an active compound, e.g., an oligonucleotide, is capable of targeting an active compound to particular cells or tissues as desired. In some embodiments, a composition for delivery of an active compound is capable of targeting an active compound to a muscle cell or tissue. In some embodiments, the present disclosure provides compositions and methods related to delivery of active compounds, wherein the compositions comprise an active compound and a lipid. In various embodiments to a muscle cell or tissue, a lipid is selected from lauric acid, myristic acid, palmitic acid, stearic acid, oleic acid, linoleic acid, alpha-linolenic acid, gamma-linolenic acid, docosahexaenoic acid (cis-DHA), turbinaric acid and dilinoleyl alcohol.

In some embodiments, a ds oligonucleotide targeting HSD17B13 is delivered via a composition comprising any one or more of, or a method of delivery involving the use of any one or more of: transferrin receptor-targeted nanoparticle; cationic liposome-based delivery strategy; cationic liposome; polymeric nanoparticle; viral carrier; retrovirus; adeno-associated virus; stable nucleic acid lipid particle; polymer; cell-penetrating peptide; lipid; dendrimer; neutral lipid; cholesterol; lipid-like molecule; fusogenic lipid; hydrophilic molecule; polyethylene glycol (PEG) or a derivative thereof; shielding lipid; PEGylated lipid; PEG-C-DMSO; PEG-C-DMSA; DSPC; ionizable lipid; a guanidinium-based cholesterol derivative; ion-coated nanoparticle; metal-ion coated nanoparticle; manganese ion-coated nanoparticle; angubindin-1; nanogel; incorporation of the HSD17B13 targeting ds oligonucleotide into a branched nucleic acid structure; and/or incorporation of the HSD17B13 targeting ds oligonucleotide into a branched nucleic acid structure comprising 2, 3, 4 or more oligonucleotides.

In some embodiments, a composition comprising an oligonucleotide is lyophilized. In some embodiments, a composition comprising an oligonucleotide is lyophilized, and the lyophilized oligonucleotide is in a vial. In some embodiments, the vial is back filled with nitrogen. In some embodiments, the lyophilized oligonucleotide composition is reconstituted prior to administration. In some embodiments, the lyophilized oligonucleotide composition is reconstituted with a sodium chloride solution prior to administration. In some embodiments, the lyophilized oligonucleotide composition is reconstituted with a 0.9% sodium chloride solution prior to administration. In some embodiments, reconstitution occurs at the clinical site for administration. In some embodiments, in a lyophilized composition, an oligonucleotide composition is chirally controlled or comprises at least one chirally controlled internucleotidic linkage and/or the ds oligonucleotide targets HSD17B13.

### Transgenic HSD17B13 Non-Human Animals

In some embodiments, the present disclosure provides engineered animals and cell thereof, wherein the animals are engineered to comprise or express an HSD17B13 polypeptide or a characteristic portion thereof, and/or a polynucleotide encoding such an HSD17B13 polypeptide or a characteristic portion thereof. In some embodiments, an HSD17B13 polypeptide or a characteristic portion thereof is or comprises a sequence that shares about 80-100%, e.g., about or at least about 80%, 81%, 82%, 83%, 84%, 85%, 86%, 87%, 88%, 89%, 90%, 91%, 92%, 93%, 94%, 95%, 96%, 97%, 98%, 99%, 100% identity with a primate, e.g., a human HSD17B13 or a characteristic portion thereof. In some embodiments, an HSD17B13 polypeptide or a characteristic portion thereof is or comprises a sequence that shares about 80-100%, e.g., about or at least about 80%, 81%, 82%, 83%, 84%, 85%, 86%, 87%, 88%, 89%, 90%, 91%, 92%, 93%, 94%, 95%, 96%, 97%, 98%, 99%, 100% identity with one or more domains of human HSD17B13 (e.g., an HSD17B13 lipid droplet targeting domain (comprising an N-terminal hydrophobic domain, PAT-like domain, and a putative a-helix/p-sheet/a-helix domain) and HSD17B13 enzymatic activity domains (comprising catalytic sites, substrate binding sites, and homodimer interaction sites).

### Exemplary HSD17B13 Sequence Embodiments

In certain embodiments, an HSD17B13 polynucleotide or a human HSD17B13 gene incorporated into a non-human animal (e.g., a rodent, e.g., a rat or mouse) is represented by or comprises a sequence encoding a human HSD17B13 or genomic locus, or a characteristic portion thereof.

As used herein, the term “characteristic portion”, in the broadest sense, refers to a portion of a substance whose presence (or absence) correlates with presence (or absence) of a particular feature, attribute, or activity of the substance. In some embodiments, a characteristic portion of a substance is a portion that is found in the substance and in related substances that share the particular feature, attribute or activity, but not in those that do not share the particular feature, attribute or activity. In certain embodiments, a characteristic portion shares at least one functional characteristic with the intact substance. For example, in some embodiments, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to the sequence and/or structural identity specified above, shares at least one functional characteristic with the relevant intact substance. In some embodiments, a characteristic portion may be biologically active.

Exemplary HSD17B13 coding sequences are described above.

### Exemplary Promoters

In some embodiments, a construct (e.g., a construct harboring human HSD17B13 gene) comprises a promoter. The term “promoter” refers to a DNA sequence recognized by enzymes/proteins that can promote and/or initiate transcription of an operably linked gene (e.g., a human HSD17B13 gene). For example, a promoter typically refers to, e.g., a nucleotide sequence to which an RNA polymerase and/or any associated factor binds and from which it can initiate transcription. Thus, in some embodiments, a construct (e.g., a targeting construct and/or vector comprising a human HSD17B13 gene) comprises a promoter operably linked to one of the non-limiting example promoters described herein.

In some embodiments, a promoter is an inducible promoter, a constitutive promoter, a mammalian cell promoter, a viral promoter, a chimeric promoter, an engineered promoter, a tissue-specific promoter, an insertional site endogenous promoter, or any other type of promoter known in the art. In some embodiments, a promoter is a RNA polymerase II promoter, such as a mammalian RNA polymerase II promoter. In some embodiments, a promoter is a RNA polymerase III promoter, including, but not limited to, a HI promoter, a human U6 promoter, a mouse U6 promoter, or a swine U6 promoter. A promoter will generally be one that is able to promote transcription in an inner ear cell. In some embodiments, a promoter is a cochlea-specific promoter or a cochlea-oriented promoter. In some embodiments, a promoter is a hair cell specific promoter, or a supporting cell specific promoter.

A variety of promoters are known in the art, which can be used herein. Non-limiting examples of promoters that can be used herein include: human EF1α, human cytomegalovirus (CMV)(U.S. Pat. No. 5,168,062), human ubiquitin C (UBC), mouse phosphoglycerate kinase 1, polyoma adenovirus, simian virus 40 (SV40), β-globin, β-actin, α-fetoprotein, γ-globin, β-interferon, γ-glutamyl transferase, mouse mammary tumor virus (MMTV), Rous sarcoma virus, rat insulin, glyceraldehyde-3-phosphate dehydrogenase, metallothionein II (MT II), amylase, cathepsin, MI muscarinic receptor, retroviral LTR (e.g., human T-cell leukemia virus HTLV), AAV ITR, interleukin-2, collagenase, platelet-derived growth factor, adenovirus 5 E2, stromelysin, murine MX gene, glucose regulated proteins (GRP78 and GRP94), α-2-macroglobulin, vimentin, MHC class I gene H-2_{K} b, HSP70, proliferin, tumor necrosis factor, thyroid stimulating hormone a gene, immunoglobulin light chain, T-cell receptor, HLA DQa and DQ, interleukin-2 receptor, MHC class II, MHC class II HLA-DRa, muscle creatine kinase, prealbumin (transthyretin), elastase I, albumin gene, c-fos, c-HA-ras, neural cell adhesion molecule (NCAM), H2B (TH2B)histone, rat growth hormone, human serum amyloid (SAA), troponin I (TN I), duchenne muscular dystrophy, human immunodeficiency virus, and Gibbon Ape Leukemia Virus (GALV) promoters. Additional examples of promoters are known in the art. See, e.g., Lodish, Molecular Cell Biology, Freeman and Company, New York 2007.

In some embodiments, a promoter is the CMV immediate early promoter. In some embodiments, the promoter is a CAG promoter or a CAG/CBA promoter. In certain embodiments, a promoter comprises a CMV/CBA enhancer/promoter construct. In certain embodiments, a promoter comprises a CAG promoter or CMV/CBA/SV-40 enhancer/promoter construct.

The term “constitutive” promoter refers to a nucleotide sequence that, when operably linked with a nucleic acid encoding a protein (e.g., a pendrin protein), causes RNA to be transcribed from the nucleic acid in a cell under most or all physiological conditions.

Examples of constitutive promoters include, without limitation, the retroviral Rous sarcoma virus (RSV) LTR promoter, the cytomegalovirus (CMV) promoter (see, e.g., Boshart et al, Cell 41:521-530, 1985), the SV40 promoter, the dihydrofolate reductase promoter, the beta-actin promoter, the phosphoglycerol kinase (PGK) promoter, and the EFl-alpha promoter (Invitrogen).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech, and Ariad. Additional examples of inducible promoters are known in the art.

Examples of inducible promoters regulated by exogenously supplied compounds include the zinc-inducible sheep metallothionein (MT) promoter, the dexamethasone (Dex)-inducible mouse mammary tumor virus (MMTV) promoter, the T7 polymerase promoter system (WO 98/10088); the ecdysone insect promoter (No et al, Proc. Natl. Acad Sci. US.A. 93:3346-3351, 1996), the tetracycline-repressible system (Gossen et al, Proc. Natl. Acad Sci. US.A. 89:5547-5551, 1992), the tetracycline-inducible system (Gossen et al, Science 268:1766-1769, 1995, see also Harvey et al, Curr. Opin. Chem. Biol. 2:512-518, 1998), the RU486-inducible system (Wang et al, Nat. Biotech. 15:239-243, 1997, and Wang et al, Gene Ther. 4:432-441, 1997), and the rapamycin-inducible system (Magari et al. J Clin. Invest. 100:2865-2872, 1997).

The term “tissue-specific” promoter refers to a promoter that is active only in certain specific cell types and/or tissues (e.g., transcription of a specific gene occurs only within cells expressing transcription regulatory and/or control proteins that bind to the tissue-specific promoter).

In some embodiments, regulatory and/or control sequences impart tissue-specific gene expression capabilities. In some cases, tissue-specific regulatory and/or control sequences bind tissue-specific transcription factors that induce transcription in a tissue-specific manner. In some embodiments, a tissue-specific promoter is a central nervous system (CNS) specific promoter. Non-limiting examples of CNS specific promoters include but are not limited to promoters or functional portions thereof for genes: AldhIII, CaMIIα, Dlxl, Dlx5/6, Gad2, GFAP, Grik4, Lepr, Nes, nNOS, Pdgfra, PLP1, Pv (Pvalb), Slc17a6, Sst, Vip, Pcp2, Slc6a3 (DAT), ePet (Fev), Npy2r, Cdh3, and/or Htr6; see e.g., Kim et al., “Mouse Cre-LoxP system: general principles to determine tissue-specific roles of target genes” Laboratory Animal Research (2018) 34(4), 147-159. In certain embodiments, a CNS specific promoter comprises, or consists of, a nucleotide sequence that is the same as, or has at least has at least 70%, 75%, 80%, 85%, 90%, 92%, 94%, 96%, 98%, or 99% homology with promoters for genes: AldhIII, CaMIIα, Dlxl, Dlx5/6, Gad2, GFAP, Grik4, Lepr, Nes, nNOS, Pdgfra, PLP1, Pv (Pvalb), Slc17a6, Sst, Vip, Pcp2, Slc6a3 (DAT), ePet (Fev), Npy2r, Cdh3, and/or Htr6.

In some embodiments, a tissue-specific promoter is an ocular cell specific promoter. Non-limiting examples of ocular cell specific promoters include but are not limited to promoters or functional portions thereof for genes: EFS, GRK1, CRX, NRL, and/or RCVRN. In certain embodiments, an ocular system specific promoter comprises, or consists of, a nucleotide sequence that is the same as, or has at least has at least 70%, 75%, 80%, 85%, 90%, 92%, 94%, 96%, 98%, or 99% homology with promoters for genes: EFS, GRK1, CRX, NRL, and/or RCVRN.

In some embodiments, a tissue-specific promoter is a hepatic system specific promoter. Non-limiting examples of hepatic system specific promoters include but are not limited promoters or functional portions thereof for genes: EFS. EF-1a, MSCV, PGK, CAG, ALB, and/or SERPINA1. In certain embodiments, a hepatic system specific promoter comprises, or consists of, a nucleotide sequence that is the same as, or has at least has at least 70%, 75%, 80%, 85%, 90%, 92%, 94%, 96%, 98%, or 99% homology with promoters for genes: EFS, EF-1a, MSCV, PGK, CAG, ALB, and/or SERPINA1.

In some embodiments, provided nucleic acid constructs comprise a promoter sequence selected from a CAG, a CBA, a CMV, or a CB7 promoter. In certain embodiments, a promoter comprises, or consists of, a nucleotide sequence that is the same as, or has at least has at least 70%, 75%, 80%, 85%, 90%, 92%, 94%, 96%, 98%, or 99% homology with a CAG, a CBA, a CMV, or a CB7 promoter.

In some embodiments of any of the nucleic acid constructs described herein, the first or sole nucleic acid constructs further includes at least one promoter sequence or functional portion thereof selected from CNS, Ocular, and/or Hepatic cell specific promoters.

### Exemplary Enhancers

In some instances, a construct can include an enhancer sequence. In some embodiments, the term “enhancer” refers to a nucleotide sequence that can increase the level of transcription of a nucleic acid encoding a protein of interest (e.g., a human and/or NHP HSD17B13 protein). Enhancer sequences (generally 50-1500 bp in length) generally increase the level of transcription by providing additional binding sites for transcription-associated proteins (e.g., transcription factors). In some embodiments, an enhancer sequence is found within an intronic sequence. Unlike promoter sequences, enhancer sequences can act at much larger distance away from the transcription start site (e.g., as compared to a promoter). Non-limiting examples of enhancers include a RSV enhancer, a CMV enhancer, and/or a SV40 enhancer. In some embodiments, a construct comprises a CMV enhancer, In some embodiments, an SV-40 derived enhancer is the SV-40 T intron sequence. In some embodiments, an enhancer sequence is woodchuck hepatitis virus post-transcriptional regulatory element (WPRE).

### Exemplary Coding Sequence Flanking Untranslated Regions

In some embodiments, any of the constructs described herein can include an untranslated region (UTR), such as a 5′ UTR or a 3′ UTR. UTRs of a gene are transcribed but not translated. A 5′ UTR starts at the transcription start site and continues to the start codon but does not include the start codon. A 3′ UTR starts immediately following the stop codon and continues until the transcriptional termination signal. The regulatory and/or control features of a UTR can be incorporated into any of the constructs, compositions, kits, or methods as described herein to enhance or otherwise modulate the expression of an HSD17B13 protein.

Natural 5′ UTRs include a sequence that plays a role in translation initiation. in some embodiments, a 5′ UTR can comprise sequences, like Kozak sequences, which are commonly known to be involved in the process by which the ribosome initiates translation of many genes. Kozak sequences have the consensus sequence CCR(A/G)CCAUGG, where R is a purine (A or G) three bases upstream of the start codon (AUG), and the start codon is followed by another “G”. In certain embodiments, a KOZAK sequence is GCCACC. The 5′ UTRs have also been known to form secondary structures that are involved in elongation factor binding.

In some embodiments, a 5′ UTR is included in any of the constructs described herein. Non-limiting examples of 5′ UTRs, including those from the following genes: albumin, serum amyloid A, Apolipoprotein A/B/E, transferrin, alpha fetoprotein, erythropoietin, Factor VIII, and HSD17B13 can be used to enhance expression of a nucleic acid molecule, such as an mRNA.

In some embodiments, a 5′ UTR from an mRNA that is transcribed by a cell in the CNS can be included in any of the constructs, compositions, kits, and methods described herein. In some embodiments, a 5′ UTR is derived from the endogenous HSD17B13 gene loci.

3′ UTRs are found immediately 3′ to the stop codon of the gene of interest. In some embodiments, a 3′ UTR from an mRNA that is transcribed by a cell in the CNS can be included in any of the constructs, compositions, kits, and methods described herein. In some embodiments, a 3′ UTR is derived from the endogenous HSD17B13 gene loci and may include all or part of the endogenous sequence.

3′ UTRs are known to have stretches of adenosines and uridines (in the RNA form) or thymidines (in the DNA form) embedded in them. These AU-rich signatures are particularly prevalent in genes with high rates of turnover. Based on their sequence features and functional properties, the AU-rich elements (AREs) can be separated into three classes (Chen et al., Mal. Cell. Biol. 15:5777-5788, 1995; Chen et al., Mal. Cell Biol. 15:2010-2018, 1995): Class I AREs contain several dispersed copies of an AUUUA motif within U-rich regions. For example, c-Myc and MyoD mRNAs contain class I AREs. Class II AREs possess two or more overlapping UUAUUUA(U/A) (U/A) nonamers. GM-CSF and TNF-alpha mRNAs are examples that contain class II AREs. Class III AREs are less well defined. These U-rich regions do not contain an AUUUA motif, two well-studied examples of this class are c-Jun and myogenin mRNAs.

Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

In some embodiments, the introduction, removal, or modification of 3′ UTR AREs can be used to modulate the stability of an mRNA encoding an HSD17B13 protein. In other embodiments, AREs can be removed or mutated to increase the intracellular stability and thus increase translation and production of an HSD17B13 protein.

In other embodiments, non-ARE sequences may be incorporated into the 5′ or 3′ UTRs. In some embodiments, introns or portions of intron sequences may be incorporated into the flanking regions of the polynucleotides in any of the constructs, compositions, kits, and methods provided herein. Incorporation of intronic sequences may increase protein production as well as mRNA levels.

### Exemplary Internal Ribosome Entry Sites (IRES)

In some embodiments, a construct encoding an HSD17B13 protein can include an internal ribosome entry site (IRES). An IRES forms a complex secondary structure that allows translation initiation to occur from any position with an mRNA immediately downstream from where the IRES is located (see, e.g., Pelletier and Sonenberg, Mal. Cell. Biol. 8(3):1103-1112, 1988).

There are several IRES sequences known to those in skilled in the art, including those from, e.g., foot and mouth disease virus (FMDV), encephalomyocarditis virus (EMCV), human rhinovirus (HRV), cricket paralysis virus, human immunodeficiency virus (HIV), hepatitis A virus (HAV), hepatitis C virus (HCV), and poliovirus (PV). See e.g., Alberts, Molecular Biology of the Cell, Garland Science, 2002; and Hellen et al., Genes Dev. 15(13):1593-612, 2001.

In some embodiments, the IRES sequence that is incorporated into a construct that encodes an HSD17B13 protein is the foot and mouth disease virus (FMDV) 2A sequence. The Foot and Mouth Disease Virus 2A sequence is a small peptide (approximately 18 amino acids in length) that has been shown to mediate the cleavage of polyproteins (Ryan, M D et al., EMBO 4:928-933, 1994; Mattion et al., J Virology 70:8124-8127, 1996; Furler et al., Gene Therapy 8:864-873, 2001; and Halpin et al., Plant Journal 4:453-459, 1999). The cleavage activity of the 2A sequence has previously been demonstrated in artificial systems including plasmids and gene therapy constructs (AAV and retroviruses) (Ryan et al., EMBO 4:928-933, 1994; Mattion et al., J Virology 70:8124-8127, 1996; Furler et al., Gene Therapy 8:864-873, 2001; and Halpin et al., Plant Journal 4:453-459, 1999; de Felipe et al., Gene Therapy 6:198-208, 1999; de Felipe et al., Human Gene Therapy I I: 1921-1931, 2000; and Klump et al., Gene Therapy 8:811-817, 2001).

An IRES can be utilized in an any constructs described herein. In some embodiments, an IRES can be part of a composition comprising more than one construct. In some embodiments, an IRES is used to produce more than one polypeptide from a single gene transcript.

### Exemplary Splice Sites

In some embodiments, any of the constructs provided herein can include splice donor and/or splice acceptor sequences, which are functional during RNA processing occurring during transcription. In some embodiments, splice sites are involved in trans-splicing.

### Exemplary Polyadenylation Sequences

In some embodiments, a construct provided herein can include a polyadenylation (poly(A)) signal sequence. Most nascent eukaryotic mRNAs possess a poly(A) tail at their 3′ end, which is added during a complex process that includes cleavage of the primary transcript and a coupled polyadenylation reaction driven by the poly(A) signal sequence (see, e.g., Proudfoot et al., Cell 108:501-512, 2002). A poly(A) tail confers mRNA stability and transferability (Molecular Biology of the Cell, Third Edition by B. Alberts et al., Garland Publishing, 1994). In some embodiments, a poly(A) signal sequence is positioned 3′ to the coding sequence.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. A 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In some embodiments, a poly(A) tail is added onto transcripts that contain a specific sequence, e.g., a poly(A) signal. A poly(A) tail and associated proteins aid in protecting mRNA from degradation by exonucleases. Polyadenylation also plays a role in transcription termination, export of the mRNA from the nucleus, and translation. Polyadenylation typically occurs in the nucleus immediately after transcription of DNA into RNA, but also can occur later in the cytoplasm. After transcription has been terminated, an mRNA chain is cleaved through the action of an endonuclease complex associated with RNA polymerase. A cleavage site is usually characterized by the presence of the base sequence AAUAAA near the cleavage site. After the mRNA has been cleaved, adenosine residues are added to the free 3′ end at the cleavage site.

As used herein, a “poly(A) signal sequence” or “polyadenylation signal sequence” is a sequence that triggers the endonuclease cleavage of an mRNA and the addition of a series of adenosines to the 3′ end of the cleaved mRNA.

There are several poly(A) signal sequences that can be used, including those derived from bovine growth hormone (bGH)(Woychik et al., Proc. Natl. Acad Sci. US.A. 81(13):3944-3948, 1984; U.S. Pat. No. 5,122,458), mouse-β-globin, mouse-a-globin (Orkin et al., EMBO J 4(2):453-456, 1985; Thein et al., Blood 71(2):313-319, 1988), human collagen, polyoma virus (Batt et al., Mal. Cell Biol. 15(9):4783-4790, 1995), the Herpes simplex virus thymidine kinase gene (HSV TK), IgG heavy-chain gene polyadenylation signal (US 2006/0040354), human growth hormone (hGH) (Szymanski et al., Mal. Therapy 15(7):1340-1347, 2007), the group consisting of SV40 poly(A) site, such as the SV40 late and early poly(A) site (Schek et al., Mal. Cell Biol. 12(12):5386-5393, 1992).

The poly(A) signal sequence can be AATAAA. The AATAAA sequence may be substituted with other hexanucleotide sequences with homology to AATAAA and that are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414).

In some embodiments, a poly(A) signal sequence can be a synthetic polyadenylation site (see, e.g., the pCl-neo expression construct of Promega that is based on Levitt el al, Genes Dev. 3(7):1019-1025, 1989). In some embodiments, a poly(A) signal sequence is the polyadenylation signal of soluble neuropilin-1 (sNRP) (AAATAAAATACGAAATG) (see, e.g., WO 05/073384). In some embodiments, a poly(A) signal sequence comprises or consists of bGHpA. In some embodiments, a poly(A) signal sequence comprises or consists of the SV40 poly(A) site. Additional examples of poly(A) signal sequences are known in the art.

### Exemplary Destabilization Domains

In some embodiments, any of the constructs provided herein can optionally include a sequence encoding a destabilizing domain (“a destabilizing sequence”) for temporal control of protein expression. Non-limiting examples of destabilizing sequences include sequences encoding a FK506 sequence, a dihydrofolate reductase (DHFR) sequence, or other exemplary destabilizing sequences.

In the absence of a stabilizing ligand, a protein sequence operatively linked to a destabilizing sequence is degraded by ubiquitination. In contrast, in the presence of a stabilizing ligand, protein degradation is inhibited, thereby allowing the protein sequence operatively linked to the destabilizing sequence to be actively expressed. As a positive control for stabilization of protein expression, protein expression can be detected by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

Additional examples of destabilizing sequences are known in the art. In some embodiments, the destabilizing sequence is a FK506- and rapamycin-binding protein (FKBP12) sequence, and the stabilizing ligand is Shield-1 (Shld1) (Banaszynski et al. (2012) Cell 126(5): 995-1004). In some embodiments, a destabilizing sequence is a DHFR sequence, and a stabilizing ligand is trimethoprim (TMP) (Iwamoto et al. (2010) Chem Biol 17:981-988).

In some embodiments, a destabilizing sequence is a FKBP12 sequence, and a presence of a nucleic acid construct carrying the FKBP12 gene in a subject cell (e.g., a rodent cell, e.g., a rat or mouse cell) is detected by western blotting. In some embodiments, a destabilizing sequence can be used to verify the temporally-specific activity of any of the nucleic acid constructs described herein.

### Exemplary Reporter Sequences or Elements

In some embodiments, constructs provided herein can optionally include a sequence encoding a reporter polypeptide and/or protein (“a reporter sequence”). Non-limiting examples of reporter sequences include DNA sequences encoding: a beta-lactamase, a beta-galactosidase (LacZ), an alkaline phosphatase, a thymidine kinase, a green fluorescent protein (GFP), a red fluorescent protein, an mCherry fluorescent protein, a yellow fluorescent protein, a chloramphenicol acetyltransferase (CAT), and a luciferase. Additional examples of reporter sequences are known in the art. When associated with control elements which drive their expression, the reporter sequence can provide signals detectable by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

In some embodiments, a reporter sequence is the LacZ gene, and the presence of a construct carrying the LacZ gene in a non-human cell (e.g., a rodent cell, e.g., a rat or mouse cell) is detected by assays for beta-galactosidase activity. When the reporter is a fluorescent protein (e.g., green fluorescent protein) or luciferase, the presence of a construct carrying the fluorescent protein or luciferase in a non-human cell (e.g., a rodent cell, e.g., a rat or mouse) may be measured by fluorescent techniques (e.g., fluorescent microscopy or FACS) or light production in a luminometer (e.g., a spectrophotometer or an IVIS imaging instrument). In some embodiments, a reporter sequence can be used to verify the tissue-specific targeting capabilities and tissue-specific promoter regulatory and/or control activity of any of the constructs described herein.

In some embodiments, a reporter sequence is a FLAG tag (e.g., a 3×FLAG tag), and the presence of a construct carrying the FLAG tag in a non-human cell (e.g., a rodent cell, e.g., a rat or mouse) is detected by protein binding or detection assays (e.g., Western blots, immunohistochemistry, radioimmunoassay (RIA), mass spectrometry).

### Targeting Vectors

Targeting vectors can be employed to introduce a nucleic acid construct into a target genomic locus. Targeting vectors can comprise a nucleic acid construct and homology arms that flank said nucleic acid construct; those skilled in the art will be aware of a variety of options and features generally applicable to the design, structure, and/or use of targeting vectors. For example, targeting vectors can be in linear form or in circular form, and they can be single-stranded or double-stranded. Targeting vectors can be deoxyribonucleic acid (DNA) or ribonucleic acid (RNA). For ease of reference, homology arms are referred to herein as 5′ and 3′ (i.e., upstream and downstream, i.e., left and right)homology arms. This terminology relates to the relative position of the homology arms to a nucleic acid construct within a targeting vector. 5′ and 3′ homology arms correspond to regions within a targeted locus or to a region within another targeting vector, which are referred to herein as “5′ target sequence” and “3′ target sequence,” respectively. In some embodiments, homology arms can also function as a 5′ or a 3′ target sequence. In some embodiments, the present disclosure provides targeting vectors comprising a provided technology whose sequence encodes an HSD17B13 polypeptide or a characteristic portion thereof as described herein.

In some embodiments, methods described herein provide for traditional transgenic non-human animal creation. In such embodiments, a vector comprising an exogenous HSD17B13 gene is injected into a zygote and integrated randomly within the genome. In some embodiments, such a random insertion site may be within a protein coding region and may result in the modification of function of an endogenous protein and/or gene. In some such embodiments, an exogenous HSD17B13 gene may be incorporated as solely a coding region, as a coding region including a protein tag, as a coding region with an operably linked promoter, as a coding region including a poly(A) site, as a coding region including any additional regulatory region, or as any combination thereof.

In some embodiments, methods described herein provide for traditional transgenic non-human animal creation utilizing the Tol2 transposon system. In such embodiments, a vector comprising an exogenous HSD17B13 gene is injected into a zygote and integrated randomly within an ANT rich region of the genome. In some embodiments, such a random insertion site may be within a protein coding region and may result in the modification of function of an endogenous protein and/or gene. In some such embodiments, an exogenous HSD17B13 gene may be incorporated as solely a coding region, as a coding region including a protein tag, as a coding region with an operably linked promoter, as a coding region including a poly(A) site, as a coding region including any additional regulatory region, or as any combination thereof. In some embodiments, methods described herein providing for traditional transgenic non-human animal creation may utilize large genomic fragments (e.g., 1 mb, 10 mb, 100 mb, and/or 1000 mb). In some embodiments, traditional transgenic non-human animals may comprise transgenic regions including promoters, introns, exons, and/or additional genomic regulatory regions. In some embodiments, traditional transgenic non-human animal creation may utilize a yeast artificial chromosome (YAC), a bacterial artificial chromosome (BAC), a human artificial chromosome, a P1-derived artificial chromosome (PAC), or any other engineered region which may be contained in an appropriate host cell.

In some embodiments, methods described herein employ two, three or more targeting vectors that are capable of recombining with each other. In some embodiments, first, second, and third targeting vectors each comprise a 5′ and a 3′ homology arm. The 3′ homology arm of the first targeting vector comprises a sequence that overlaps with the 5′ homology arm of the second targeting vector (i.e., overlapping sequences), which allows for homologous recombination between first and second vector.

In some embodiments of double component targeting methods, a 5′homology arm of a first targeting vector and a 3′ homology arm of a second targeting vector can be similar to corresponding segments within a target genomic locus (i.e., a target sequence), which can promote homologous recombination of the first and the second targeting vectors with corresponding genomic segments and modify the target genomic locus.

In some embodiments of triple component targeting methods, a 3′ homology arm of a second targeting vector can comprise a sequence that overlaps with a 5′ homology arm of a third targeting vector (i.e., overlapping sequences), which can allow for homologous recombination between the second and the third targeting vector. The 5′ homology arm of the first targeting vector and the 3′ homology arm of the third targeting vector are similar to corresponding segments within the target genomic locus (i.e., the target sequence), which can promote homologous recombination of the first and the third targeting vectors with the corresponding genomic segments and modify the target genomic locus.

In some embodiments, a homology arm and a target sequence or two homology arms “correspond” or are “corresponding” to one another when the two regions share a sufficient level of sequence identity to one another so that they can act as substrates for a homologous recombination reaction. The sequence identity between a given target sequence and the corresponding homology arm found on a targeting vector (i.e., overlapping sequence) or between two homology arms can be any degree of sequence identity that allows for homologous recombination to occur. To give but one example, an amount of sequence identity shared by a homology arm of a targeting vector (or a fragment thereof) and a target sequence of another targeting vector or a target sequence of a target genomic locus (or a fragment thereof) can be, e.g., but not limited to, at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination.

Moreover, a corresponding region of similarity (e.g., identity) between a homology arm and a corresponding target sequence can be of any length that is sufficient to promote homologous recombination at the target genomic locus. For example, a given homology arm and/or corresponding target sequence can comprise corresponding regions of similarity that are but are not limited to, about 0.2-0.5 kb, 0.2-1 kb, 0.2-1.5 kb, 0.2-2 kb, 0.2-2.5 kb, 0.2-3 kb, 0.2-3.5 kb, 0.2-4 kb, 0.2-4.5 kb, or 0.2-5 kb in length such that a homology arm has sufficient similarity to undergo homologous recombination with a corresponding target sequence(s) within a target genomic locus of the cell or within another targeting vector. In some embodiments, a given homology arm and/or corresponding target sequence can comprise corresponding regions of similarity that are, e.g., but not limited to, about 5-10 kb, 5-15 kb, 5-20 kb, 5-25 kb, 5-30 kb, 5-35 kb, 5-40 kb, 5-45 kb, 5-50 kb, 5-55 kb, 5-60 kb, 5-65 kb, 5-70 kb, 5- 75 kb, 5-80 kb, 5-85 kb, 5-90 kb, 5-95 kb, 5-100 kb, 100-200 kb, or 200-300 kb in length (such as described elsewhere herein) such that a homology arm has sufficient similarity to undergo homologous recombination with a corresponding target sequence(s) within a target genomic locus of the cell or within another targeting vector. In some embodiments, a given homology arm and/or corresponding target sequence comprise corresponding regions of similarity that are, e.g., but not limited to, about 10-100 kb, 15-100 kb, 20-100 kb, 25-100 kb, 30-100 kb, 35-100 kb, 40-100 kb, 45-100 kb, 50-100 kb, 55-100 kb, 60-100 kb, 65-100 kb, 70-100 kb, 75-100 kb, 80-100 kb, 85-100 kb, 90-100 kb, or 95-100 kb in length (such as described elsewhere herein) such that a homology arm has sufficient similarity to undergo homologous recombination with a corresponding target sequence(s) within a target genomic locus of the cell or within another targeting vector.

In some embodiments, overlapping sequences of a 3′ homology arm of a first targeting vector and a 5′ homology arm of a second targeting vector or of a 3′ homology arm of a second targeting vector and a 5′ homology arm of a third targeting vector can be of any length that is sufficient to promote homologous recombination between said targeting vectors. For example, a given homology arm and/or corresponding target sequence can comprise corresponding regions of similarity that are, e.g., but not limited to, about 0.2-0.5 kb, 0.2-1 kb, 0.2-1.5 kb, 0.2-2 kb, 0.2-2.5 kb, 0.2-3 kb, 0.2-3.5 kb, 0.2-4 kb, 0.2-4.5 kb, or 0.2-5 kb in length such that a homology arm has sufficient similarity to undergo homologous recombination with a corresponding target sequence(s) within a target genomic locus of the cell or within another targeting vector. In some embodiments, a given overlapping sequence of a homology arm can comprise corresponding overlapping regions that are about 1-5 kb, 5-10 kb, 5-15 kb, 5-20 kb, 5-25 kb, 5-30 kb, 5-35 kb, 5-40 kb, 5-45 kb, 5-50 kb, 5-55 kb, 5-60 kb, 5-65 kb, 5-70 kb, 5-75 kb, 5- 80 kb, 5-85 kb, 5-90 kb, 5-95 kb, 5-100 kb, 100-200 kb, or 200-300 kb in length such that an overlapping sequence of a homology arm has sufficient similarity to undergo homologous recombination with a corresponding overlapping sequence within another targeting vector. In some embodiments, a given overlapping sequence of a homology arm comprises an overlapping region that is about 1-100 kb, 5-100 kb, 10-100 kb, 15-100 kb, 20-100 kb, 25-100 kb, 30-100 kb, 35-100 kb, 40-100 kb, 45-100 kb, 50-100 kb, 55-100 kb, 60-100 kb, 65-100 kb, 70-100 kb, 75-100 kb, 80-100 kb, 85-100 kb, 90-100 kb, or 95-100 kb in length such that an overlapping sequence of a homology arm has sufficient similarity to undergo homologous recombination with a corresponding overlapping sequence within another targeting vector. In some embodiments, an overlapping sequence is from 1-5 kb, inclusive. In some embodiments, an overlapping sequence is from about 1 kb to about 70 kb, inclusive. In some embodiments, an overlapping sequence is from about 10 kb to about 70 kb, inclusive. In some embodiments, an overlapping sequence is from about 10 kb to about 50 kb, inclusive. In some embodiments, an overlapping sequence is at least 10 kb. In some embodiments, an overlapping sequence is at least 20 kb. For example, an overlapping sequence can be from about 1 kb to about 5 kb, inclusive, from about 5 kb to about 10 kb, inclusive, from about 10 kb to about 15 kb, inclusive, from about 15 kb to about 20 kb, inclusive, from about 20 kb to about 25 kb, inclusive, from about 25 kb to about 30 kb, inclusive, from about 30 kb to about 35 kb, inclusive, from about 35 kb to about 40 kb, inclusive, from about 40 kb to about 45 kb, inclusive, from about 45 kb to about 50 kb, inclusive, from about 50 kb to about 60 kb, inclusive, from about 60 kb to about 70 kb, inclusive, from about 70 kb to about 80 kb, inclusive, from about 80 kb to about 90 kb, inclusive, from about 90 kb to about 100 kb, inclusive, from about 100 kb to about 120 kb, inclusive, from about 120 kb to about 140 kb, inclusive, from about 140 kb to about 160 kb, inclusive, from about 160 kb to about 180 kb, inclusive, from about 180 kb to about 200 kb, inclusive, from about 200 kb to about 220 kb, inclusive, from about 220 kb to about 240 kb, inclusive, from about 240 kb to about 260 kb, inclusive, from about 260 kb to about 280 kb, inclusive, or about 280 kb to about 300 kb, inclusive. To give but one example, an overlapping sequence can be from about 20 kb to about 60 kb, inclusive. Alternatively, an overlapping sequence can be at least 1 kb, at least 5 kb, at least 10 kb, at least 15 kb, at least 20 kb, at least 25 kb, at least 30 kb, at least 35 kb, at least 40 kb, at least 45 kb, at least 50 kb, at least 60 kb, at least 70 kb, at least 80 kb, at least 90 kb, at least 100 kb, at least 120 kb, at least 140 kb, at least 160 kb, at least 180 kb, at least 200 kb, at least 220 kb, at least 240 kb, at least 260 kb, at least 280 kb, or at least 300 kb. In some embodiments, an overlapping sequence can be at most 400 kb, at most 350 kb, at most 300 kb, at most 280 kb, at most 260 kb, at most 240 kb, at most 220 kb, at most 200 kb, at most 180 kb, at most 160 kb, at most 140 kb, at most 120 kb, at most 100 kb, at most 90 kb, at most 80 kb, at most 70 kb, at most 60 kb or at most 50 kb.

### Exemplary Sites For Genomic Incorporation

Homology arms can, in some embodiments, correspond to a locus that is native to a cell (e.g., a targeted locus), or alternatively they can correspond to a region of a heterologous or exogenous segment of DNA that was integrated into the genome of the cell, including, for example, transgenes, expression cassettes, or heterologous or exogenous regions of DNA. In some embodiments, homology arms can, correspond to a region on a targeting vector in a cell. In some embodiments, homology arms of a targeting vector may correspond to a region of a yeast artificial chromosome (YAC), a bacterial artificial chromosome (BAC), a human artificial chromosome, a P1-derived artificial chromosome (PAC), or any other engineered region contained in an appropriate host cell. Still further, homology arms of a targeting vector may correspond to or be derived from a region of a BAC library, a cosmid library, or a P1 phage library. In some certain embodiments, homology arms of a targeting vector correspond to a locus that is native, heterologous, or exogenous to a prokaryote, a yeast, a bird (e.g., chicken), a non-human mammal, a rodent, a human, a rat, a mouse, a hamster a rabbit, a pig, a bovine, a deer, a sheep, a goat, a cat, a dog, a ferret, a primate (e.g., marmoset, rhesus monkey), a domesticated mammal, an agricultural mammal, or any other organism of interest. In some embodiments, homology arms correspond to a locus of the cell that shows limited susceptibility to targeting using a conventional method or that has shown relatively low levels of successful integration at a targeted site, and/or significant levels of off-target integration, in the absence of a nick or double-strand break induced by a nuclease agent (e.g., a Cas protein, a Zinc Finger nuclease protein, and/or a TALEN protein). In some embodiments, homology arms are designed to include engineered DNA.

In some embodiments, 5′ and 3′ homology arms of a targeting vector(s) correspond to a targeted genome. Alternatively, homology arms correspond to a related genome. For example, a targeted genome is a mouse genome of a first strain, and targeting arms correspond to a mouse genome of a second strain, wherein the first strain and the second strain are different.

In certain embodiments, homology arms correspond to the genome of the same animal or are from the genome of the same strain, e.g., the targeted genome is a mouse genome of a first strain, and the targeting arms correspond to a mouse genome from the same mouse or from the same strain.

A homology arm of a targeting vector can be of any length that is sufficient to promote a homologous recombination event with a corresponding target sequence, including, for example, 0.2-1 kb, inclusive, 1-5 kb, inclusive, 5-10 kb, inclusive, 5-15 kb, inclusive, 5-20 kb, inclusive, 5-25 kb, inclusive, 5-30 kb, inclusive, 5-35 kb, inclusive, 5-40 kb, inclusive, 5-45 kb, inclusive, 5-50 kb, inclusive, 5-55 kb, inclusive, 5-60 kb, inclusive, 5-65 kb, inclusive, 5-70 kb, inclusive, 5-75 kb, inclusive, 5-80 kb, inclusive, 5-85 kb, inclusive, 5-90 kb, inclusive, 5-95 kb, inclusive, 5-100 kb, inclusive, 100-200 kb, inclusive, or 200-300 kb, inclusive, in length. In some embodiments, a homology arm of a targeting vector has a length that is sufficient to promote a homologous recombination event with a corresponding target sequence that is 0.2-100 kb, inclusive, 1-100 kb, inclusive, 5-100 kb, inclusive, 10-100 kb, inclusive, 15-100 kb, inclusive, 20-100 kb, inclusive, 25-100 kb, inclusive, 30-100 kb, inclusive, 35-100 kb, inclusive, 40-100 kb, inclusive, 45-100 kb, inclusive, 50-100 kb, inclusive, 55-100 kb, inclusive, 60-100 kb, inclusive, 65-100 kb, inclusive, 70-100 kb, inclusive, 75-100 kb, inclusive, 80-100 kb, inclusive, 85-100 kb, inclusive, 90-100 kb, inclusive, or 95-100 kb, inclusive, in length. As described herein, large targeting vectors can employ targeting arms of greater length.

In certain embodiments, an HSD17B13 polynucleotide or an exogenous HSD17B13 locus is incorporated into a non-human animal (e.g., a rodent, e.g., a rat or mouse) at an endogenous locus. In some cases, an endogenous locus is an HSD17B13 locus. In some such cases, an endogenous HSD17B13 locus may be replaced with an exogenous HSD17B13 gene. In some embodiments, replacement may be partial, or may be complete. In some embodiments, an HSD17B13 polynucleotide or an exogenous HSD17B13 gene is incorporated into an endogenous HSD17B13 locus and is operably linked to an endogenous HSD17B13 promoter.

In certain embodiments, an HSD17B13 polynucleotide or an exogenous HSD17B13 locus is incorporated into a non-human animal (e.g., a rodent, e.g., a rat or mouse) at an endogenous locus. In some cases, an endogenous locus is a locus driven by a constitutive promoter. In some embodiments, an endogenous locus is a locus driven by a tissue specific promoter.

In certain embodiments, an HSD17B13 polynucleotide or an exogenous HSD17B13 gene is incorporated into a non-human animal (e.g., a rodent, e.g., a rat or mouse) at a site amenable to Cre/LoxP manipulation. In certain embodiments, an HSD17B13 polynucleotide or an exogenous HSD17B13 is incorporated into a site or is located within a targeting vector flanked by LoxP recombination sites. In certain embodiments, a non-human animal (e.g., a rodent, e.g., a rat or mouse) with an exogenous HSD17B13 gene comprising or incorporated into a site flanked by LoxP sites can further be crossed with an animal expressing a Cre recombinase under the control of one or more of a tissue specific, temporally specific, and/or inducible promoter.

In certain embodiments, an HSD17B13 polynucleotide or an exogenous HSD17B13 gene is incorporated into a non-human animal (e.g., rodent, e.g., rat or mouse), non-human (e.g., rodent, e.g., rat or mouse) cell or non-human (e.g., rodent, e.g., rat or mouse) tissue at a locus amenable for manipulation using Cre-Lox P and/or Flp-FRT; see E.g., Kim et al., “Mouse Cre-LoxP system: general principles to determine tissue-specific roles of target genes” Laboratory Animal Research (2018) 34(4), 147-159.

In certain embodiments, an HSD17B13 polynucleotide or an exogenous HSD17B13 gene is incorporated into a non-human animal (e.g., rodent, e.g., rat or mouse), non-human (e.g., rodent, e.g., rat or mouse) cell or non-human (e.g., rodent, e.g., rat or mouse) tissue at a Cre/LoxP stop or inducible Cre/LoxP site. In certain such embodiments, when crossed with a mouse that has Cre under a tissue specific promoter, said locus can generate tissue specific exogenous HSD17B13 expression in transgenic animals.

In some embodiments of a non-human animal (e.g., rodent, e.g., rat or mouse), non-human (e.g., rodent, e.g., rat or mouse) cell or non-human (e.g., rodent, e.g., rat or mouse) tissue as described herein, the non-human animal, non-human cell or non-human tissue is homozygous or heterozygous for an exogenous HSD17B13 gene integrated at a site operably linked to an inducible promoter (e.g., a tetracycline-responsive element, an estrogen receptor targeting motif, and/or under the control of tamoxifen).

In certain embodiments, an HSD17B13 polynucleotide or an exogenous HSD17B13 gene is incorporated into a non-human animal (e.g., rodent, e.g., rat or mouse), non-human (e.g., rodent, e.g., rat or mouse) cell or non-human (e.g., rodent, e.g., rat or mouse) tissue at a locus known to function as a transcriptional hotspot, and/or transcriptional safe harbor (such as are abundant and well known in the art).

In certain embodiments, an HSD17B13 polynucleotide or an exogenous HSD17B13 gene is incorporated into a non-human animal (e.g., rodent, e.g., rat or mouse), non-human (e.g., rodent, e.g., rat or mouse) cell or non-human (e.g., rodent, e.g., rat or mouse) tissue at the ROSA26 locus.

In certain embodiments, an HSD17B13 polynucleotide or an exogenous HSD17B13 gene is incorporated into a non-human animal (e.g., rodent, e.g., rat or mouse), non-human (e.g., rodent, e.g., rat or mouse) cell or non-human (e.g., rodent, e.g., rat or mouse) tissue at the H11 locus.

In certain embodiments, an HSD17B13 polynucleotide or an exogenous HSD17B13 gene is incorporated into a non-human animal (e.g., rodent, e.g., rat or mouse), non-human (e.g., rodent, e.g., rat or mouse) cell or non-human (e.g., rodent, e.g., rat or mouse) tissue at the TIGRE locus.

In certain embodiments, an HSD17B13 polynucleotide or an exogenous HSD17B13 gene is incorporated into a non-human animal (e.g., rodent, e.g., rat or mouse), non-human (e.g., rodent, e.g., rat or mouse) cell or non-human (e.g., rodent, e.g., rat or mouse) tissue at the MYH9 locus.

In some embodiments of a non-human animal (e.g., rodent, e.g., rat or mouse), non-human (e.g., rodent, e.g., rat or mouse) cell or non-human (e.g., rodent, e.g., rat or mouse) tissue as described herein, the non-human animal, non-human cell or non-human tissue is homozygous or heterozygous for an HSD17B13 polynucleotide or an exogenous HSD17B13 gene integrated at a site operably linked to a universally expressed promoter (e.g., CMV, SV40, elongation factor 1 alpha, CBA/CAGG, ubiquitin C, and/or phosphoglycerate kinase 1).

### Nuclease Agents Facilitating Targeting Vector Integration

In some embodiments, nuclease agents (e.g., CRISPR/Cas systems, Zinc Finger Nucleases, and/or TALENs) can be employed in combination with targeting vectors to facilitate the modification of a target locus (e.g., modification of an HSD17B13 locus and/or modification of a locus targeted for exogenous protein insertion). Such nuclease agents and their use are well known in the art, and may promote homologous recombination between a targeting vector and a target locus. When nuclease agents are employed in combination with a targeting vector, the targeting vector can comprise 5′ and 3′ homology arms corresponding to 5′ and 3′ target sequences located in sufficient proximity to a nuclease cleavage site so as to promote the occurrence of a homologous recombination event between target sequences and homology arms upon a nick or double-strand break at the nuclease cleavage site. In some embodiments, the term “nuclease cleavage site” includes a DNA sequence at which a nick or double-strand break is created by a nuclease agent (e.g., a Cas9 cleavage site). Target sequences within a targeted locus that correspond to 5′ and 3′ homology arms of a targeting vector are “located in sufficient proximity” to a nuclease cleavage site if the distance is such as to promote the occurrence of a homologous recombination event between 5′ and 3′ target sequences and homology arms upon a nick or double-strand break at the recognition site. Thus, in certain embodiments, target sequences corresponding to 5′ and/or 3′ homology arms of a targeting vector are within at least one nucleotide of a given recognition site or are within at least 10 nucleotides to about 14 kb of a given recognition site. In some embodiments, a nuclease cleavage site is immediately adjacent to at least one, two, three, four, and/or more target sequences.

The spatial relationship of target sequences that correspond to homology arms of a targeting vector and a nuclease cleavage site can vary. For example, target sequences can be located 5′ to a nuclease cleavage site, target sequences can be located 3′ to a recognition site, or target sequences can flank a nuclease cleavage site.

Combined use of a targeting vector with a nuclease agent can result in an increased targeting efficiency compared to use of a targeting vector alone. For example, when a targeting vector is used in conjunction with a nuclease agent, targeting efficiency of a targeting vector can be increased by at least two-fold, at least three-fold, at least four-fold, at least five-fold, at least six-fold, at least seven-fold, at least eight-fold, at least nine-fold, at least ten-fold or within a range formed from these integers, such as 2-10-fold when compared to use of a targeting vector alone.

In some embodiments, targeting vectors comprise homology arms that correspond to and are derived from nucleic acid sequences larger than those typically used by other approaches intended to perform homologous recombination in cells. In some embodiments, targeting vectors comprise homology arms that correspond to and are derived from nucleic acid sequences shorter than those typically used by other approaches intended to perform homologous recombination in cells. In some embodiments, a homology arm is at least 10 kb in length, or the sum total of a 5′ homology arm and a 3′ homology arm can be, for example, at least 10 kb. In some embodiments, a homology arm is less than 10 kb in length, or the sum total of a 5′ homology arm and a 3′ homology arm can be, for example, is less than 10 kb. In some embodiments, targeting vectors comprising nucleic acid constructs larger than those typically used by other approaches intended to perform homologous recombination in cells. For example, in some embodiments, large loci that cannot traditionally be accommodated by plasmid-based targeting vectors because of their size limitations may still be employed through the use of large targeting vectors. For example, a targeted locus can be (i.e., 5′ and 3′ homology arms can correspond to) a locus of a cell that is not targetable using a conventional method or that can be targeted only incorrectly or only with significantly low efficiency in the absence of a nick or double-strand break induced by a nuclease agent (e.g., a Cas protein). In some embodiments, a large targeting vector may include vectors derived from bacterial artificial chromosome (BAC), a human artificial chromosome, or a yeast artificial chromosome (YAC). Large targeting vectors can be in linear form or in circular form. Examples of large targeting vectors and methods for making them are described, e.g., in Macdonald (2014), U.S. Pat. Nos. 6,586,251, 6,596,541 and 7,105,348; and International Patent Application Publication No. WO 2002/036789.

### Methods of Making Provided Non-Human Animals

Those skilled in the art appreciate that various technologies can be utilized to make engineered cells, issues, animals, etc. in accordance with the present disclosure. Provided herein are compositions and methods for making non-human animals (e.g., rodents, e.g., mice) whose germline genome comprises an engineered human HSD17B13 gene.

In some embodiments, the non-human HSD17B13 locus may be the site for insertion of a human HSD17B13 gene. In some embodiments, any suitable integration locus may be the site for insertion of a human HSD17B13 coding sequence.

In some embodiments, a human HSD17B13 gene may be under the control of a heterologous protein enhancer(s) and/or promoter(s). In some embodiments, methods described herein comprise inserting a single human HSD17B13 gene encoding a human HSD17B13 protein. In some embodiments, methods described herein comprise inserting more than one human HSD17B13 gene encoding more than one human HSD17B13 polypeptides.

Provided herein are compositions and methods for making non-human animals (e.g., rodents, e.g., mice) whose germline genome comprises an engineered non-human primate (NHP) HSD17B13 locus that includes one or more functional HSD17B13 domains (e.g., a lipid droplet targeting domain (comprising an N-terminal hydrophobic domain, PAT-like domain, and a putative a-helix/p-sheet/a-helix domain) and enzymatic activity domains (comprising catalytic sites, substrate binding sites, and homodimer interaction sites). In some embodiments, methods described herein comprise inserting transcriptionally independent portions of a NHP HSD17B13 protein which may be rejoined in vivo through the action of trans splice acceptors and/or donors.

In some embodiments, the non-human HSD17B13 locus may be the site for insertion of a NHP HSD17B13 gene. In some embodiments, any suitable integration locus may be the site for insertion of a NHP HSD17B13 coding sequence.

In some embodiments, a NHP HSD17B13 gene may be under the control of a heterologous protein enhancer(s) and/or promoter(s). In some embodiments, methods described herein comprise inserting a single NHP HSD17B13 gene encoding a NHP HSD17B13 protein. In some embodiments, methods described herein comprise inserting more than one NHP HSD17B13 gene encoding a NHP HSD17B13 polypeptide.

### Embryonic Stem Cell Modification for Creating Transgenic Animals

In some embodiments, methods of making a provided non-human animal include insertion of genetic material that comprises an exogenous HSD17B13 gene into an embryonic stem cell of a non-human animal (e.g., a rodent, e.g., a rat or mouse). In some embodiments, methods include multiple insertions in a single ES cell clone. In some embodiments, methods include sequential insertions made in a successive ES cell clones. In some embodiments, methods include a single insertion made in an engineered ES cell clone.

In some embodiments, methods of making a non-human transgenic animal involving the use of an embryonic stem cell can have a targeting vector and/or nucleic acid construct introduced through any manner known in the art. In some embodiments, a transgene is introduced to an embryonic stem cell through a method comprising but not limited to: electroporation, lipid based transfection, lipid based nanoparticles, retroviral infection, and/or lentiviral infection.

In some embodiments of a method of making a non-human animal (e.g., rodent, e.g., mouse), a DNA fragment is introduced into a non-human embryonic stem cell.

In some embodiments, methods comprising the use of embryonic stem cell modification for the creation of transgenic animals may utilize any molecular biology technique or reagent described herein.

In some embodiments, a targeting vector comprising an HSD17B13 coding sequence is electroporated into mouse ES cells, using methods known in the art. Screening and/or selection for clones that have undergone homologous recombination yields modified ES cells for generating chimeric mice that express huHSD17B13 1. Positive ES cell clones are confirmed by PCR screening using primers and probes specific for the huHSD17B13 transgene. Primers and probes vary dependent upon the insertion loci of interest. Targeted ES cells are used as donor ES cells and introduced into an 8-cell stage mouse embryo using an appropriate method (e.g., by the VELOCIMOUSE® method (see, e.g., U.S. Pat. No. 7,294,754 and Poueymirou et al. (2007). F0 generation mice that are essentially fully derived from the donor gene-targeted ES cells allowing immediate phenotypic analyses, Nature Biotech. 25(1): 91-99). Transgenic mice expressing huHSD17B13 are identified by genotyping using methods known in the art. Mice are bred to stable heterozygotic and/or homozygotic transgenic transmission of a huHSD17B13 insertion locus.

### Zygotic Injection for Creating Transgenic Animals

Where appropriate, an exogenous HSD17B13 gene (e.g., a human HSD17B13 gene encoding a human HSD17B13 protein) may separately be modified to include codons that are optimized for expression in a non-human animal (e.g., see U.S. Pat. Nos. 5,670,356 and 5,874,304). Codon optimized sequences are engineered sequences, and preferably encode the identical polypeptide (or a biologically active fragment of a characteristic portion of the polypeptide which has substantially the same activity as the full-length polypeptide) encoded by the non-codon optimized parent polynucleotide. In some embodiments, an exogenous HSD17B13 gene encoding an exogenous HSD17B13 protein may separately include an altered sequence to optimize codon usage for a particular cell type (e.g., a rodent cell, e.g., a mouse cell). For example, the codons of each nucleotide sequence to be inserted into the genome of a non-human animal as described herein (e.g., a rodent, e.g., mouse) may be optimized for expression in a cell of the non-human animal. Such a sequence may be described as a codon-optimized sequence.

In some embodiments, insertion of nucleotide sequences encoding an exogenous HSD17B13 gene employs a minimal amount of modification of the germline genome of a non-human animal as described herein and results in expression of an exogenous HSD17B13 gene (e.g., a human HSD17B13 gene or a NHP HSD17B13 gene). Methods for generating engineered non-human animals (e.g., rodents, e.g., rats or mice), including knockouts and knock-ins, are known in the art (see, e.g., Gene Targeting: A Practical Approach, Joyner, ed., Oxford University Press, Inc., 2000). For example, generation of genetically engineered rodents may optionally involve disruption of the genetic loci of one or more endogenous rodent genes (or gene segments) and introduction of one or more heterologous genes (or gene segments or nucleotide sequences) into the rodent genome, in some embodiments, at the same location as an endogenous rodent gene (or gene segments). In some embodiments, nucleotide sequences encoding an exogenous HSD17B13 gene (e.g., a human HSD17B13 gene or a NHP HSD17B13 gene) is randomly inserted in the germline genome of a rodent. In some embodiments, nucleotide sequences encoding an exogenous HSD17B13 gene are introduced upstream of a non-human (e.g., rodent, e.g., rat or mouse) HSD17B13 locus in the germline genome of a rodent; in some certain embodiments, an endogenous HSD17B13 locus is altered, modified, or engineered to contain human and/or NHP HSD17B13 gene segments, wherein any combination of HSD17B13 gene segments derived from rodent, human, and/or NHP may be utilized.

Once produced, a targeting vector can be linearized injected into a rodent zygote or alternatively electroporated into rodent embryonic stem (ES) cells to create a rodent whose germline genome comprises the exogenous HSD17B13 gene. In some embodiments, confirmation of rodent ES cells comprising a targeting vector comprising an exogenous HSD17B13 gene can be selected and/or screened for using methods known in the art. As described in the examples section below, rodent zygotes comprising an injected nucleic acid construct comprising a targeting vector comprising an exogenous HSD17B13 gene can be utilized for creating transgenic non-human animals comprising an integrated exogenous HSD17B13 gene, such animals can be screened for from a population of viable injected zygotes which have been transplanted into a surrogate mother.

In some embodiments, a targeting vector is introduced into non-human (e.g., rodent, e.g., mouse or rat) embryonic cells (e.g., zygotes and/or stem cells) by electroporation so that the sequence contained in the targeting vector results in the capacity of a non-human (e.g., rodent, e.g., rat or mouse) cell or non-human animal (e.g., a rodent, e.g., rat or mouse) to expresses an exogenous HSD17B13 gene. As described herein, a genetically engineered non-human animal is generated where an exogenous HSD17B13 gene has been created and/or incorporated into the germline genome of the non-human animal (e.g., at a defined locus, and/or at a random locus). In some embodiments, insertion and/or expression of an exogenous HSD17B13 gene is confirmed using methods known in the art (e.g., PCR, western blotting etc.) In some embodiments, oligonucleotides as described herein are then characterized in vitro or in vivo using tissues, cells, and/or animals derived from a non-human embryonic stem cell comprising an exogenous HSD17B13 gene.

In some embodiments, a method of making a genetically modified non-human animal (e.g., rodent, e.g., mouse) comprises engineering a human HSD17B13 gene in the germline genome of the non-human animal to comprise a sequence operably linked to a tissue specific regulatory region.

In some embodiments, a method of making a genetically modified non-human animal (e.g., rodent, e.g., mouse) comprises engineering a human HSD17B13 gene in the germline genome of the non-human animal to comprise a sequence operably linked to a temporally specific regulatory region.

In some embodiments, a method of making a genetically modified non-human animal (e.g., rodent, e.g., mouse) comprises engineering a human HSD17B13 gene in the germline genome of the non-human animal to comprise a sequence operably linked to a substrate specific regulatory region.

In some embodiments, a non-human animal (e.g., rodent, e.g., rat or mouse) made, generated, produced, obtained or obtainable from a method as described herein is provided. In some embodiments of a method of making a non-human animal (e.g., rodent, e.g., rat or mouse), a DNA fragment is introduced into a non-human embryonic stem cell and/or zygote whose germline genome comprises an endogenous HSD17B13 loci. Alternatively, and/or additionally, in some embodiments, the germline genome of a non-human animal (e.g., rodent, e.g., rat or mouse) as described herein further comprises a deleted, inactivated, functionally silenced or otherwise non-functional endogenous HSD17B13 locus. Genetic modifications to delete or render non-functional a gene or genetic locus may be achieved using methods described herein and/or methods known in the art.

A genetically engineered founder non-human animal (e.g., rodent, e.g., rat or mouse) can be identified based upon the presence of an exogenous HSD17B13 gene as described herein in its germline genome and/or expression of exogenous HSD17B13 protein in tissues or cells of the non-human animal. A genetically engineered founder non-human animal can then be used to breed additional non-human animals carrying an exogenous HSD17B13 gene, thereby creating a cohort of non-human animals each carrying one or more copies of an exogenous HSD17B13 gene. Moreover, genetically engineered non-human animals carrying an exogenous HSD17B13 gene can further be bred to other genetically engineered non-human animals carrying other transgenes (e.g., human disease genes) or other mutated endogenous loci as desired.

Genetically engineered non-human animals (e.g., rodents, e.g., rats or mice) may also be produced to contain selected systems that allow for regulated, directed, inducible and/or cell-type specific expression of a transgene or integrated sequence(s). For example, non-human animals as described herein may be engineered to contain one or more sequences encoding an exogenous HSD17B13 gene that is/are conditionally expressed (e.g., reviewed in Rajewski, K. et al., 1996, J. Clin. Invest. 98(3):600-3). Exemplary systems include the Cre/loxP recombinase system of bacteriophage P1 (see, e.g., Lakso, M. et al., 1992, Proc. Natl. Acad. Sci. U.S.A. 89:6232-6) and the FLP/Frt recombinase system of S. cerevisiae (O'Gorman, S. et al, 1991, Science 251:1351-5). Such animals can be provided through the construction of “double” genetically engineered animals, e.g., by mating two genetically engineered animals, one containing a transgene comprising a selected modification (e.g., an exogenous HSD17B13 gene as described herein) and the other containing a transgene encoding a recombinase (e.g., a Cre recombinase).

Non-human Animals Amenable to Exogenous HSD17B13 gene Expression

Non-human animals (e.g., rodents, e.g., rats or mice) as described herein may be prepared as described above, or using methods known in the art, to comprise additional human, humanized or otherwise engineered genes, oftentimes depending on the intended use of the non-human animal. Genetic material of such human, humanized or otherwise engineered genes may be introduced through the further alteration of the genome of cells (e.g., embryonic stem cells, and/or injection of zygotes derived from transgenic rodents comprising an exogenous HSD17B13 gene)having the genetic modifications or alterations as described above or through breeding techniques known in the art with other genetically modified or engineered strains as desired.

As those skilled in the art appreciate, various compatible mouse strains (e.g., WT, harboring one or more transgenes, containing one or more mutations in an endogenous loci, etc.), can be bred to any one of the engineered mice described herein to create any number of genetically modified mouse strains expressing an HSD17B13 (e.g., a NHP HSD17B13, a human HSD17B13, etc.) polypeptide or a characteristic portion thereof and any additional genetic features (e.g., natural mouse mutant loci, disease modelling endogenous mouse gene mutant loci, transgenically derived mutant animals expressing a human gene mutation of interest, etc.). Various technologies can be utilized to generate mice heterozygous or homozygous for a transgenic polynucleotide encoding an HSD17B13 polypeptide or a characteristic portion thereof as described herein (e.g., human HSD17B13). In some embodiments, genetically modified mice which are homozygous or heterozygous for huHSD17B13 (e.g., those described in the Examples) are bred to mice homozygous or heterozygous for a mutation (deletion, gain of function, loss of function, etc.) of an endogenous mouse gene of interest that may be associated with HSD17B13 function. Resultant progeny expressing desired HSD17B13 or characteristic portions thereof and heterozygous for a gene of interest are crossed to obtain mice homozygous and/or heterozygous for HSD17B13 and/or the gene of interest. In some embodiments, breeding may be performed by a commercial breeder (e.g., The Jackson Laboratory). In certain embodiments, mice heterozygous for a transgenic HSD17B13 insertion (e.g., as described herein) are crossed to a balancer line to maintain stable heterozygotic transgenic HSD17B13 transmission. In some embodiments, a closely linked phenotypically detectable marker is genetically engineered into transgenic HSD17B13 mice to aid with crossing and/or genotyping.

Although embodiments describing the construction of an exogenous HSD17B13 gene in a mouse (i.e., a mouse with an exogenous HSD17B13 gene integrated into its germline genome) are extensively discussed herein, other non-human animals that comprise an exogenous HSD17B13 gene are also provided. Such non-human animals include any of those which can be genetically modified to express exogenous HSD17B13 polypeptides and/or fragments thereof as described herein, including, e.g., mammals, e.g., mouse, rat, rabbit, pig, bovine (e.g., cow, bull, buffalo), deer, sheep, goat, chicken, cat, dog, ferret, primate (e.g., marmoset, rhesus monkey), etc. For example, for those non-human animals for which suitable genetically modifiable ES cells are not readily available, other methods are employed to make a non-human animal comprising the genetic modification. Such methods include, e.g., modifying a non-ES cell genome (e.g., a fibroblast or an induced pluripotent cell) and employing somatic cell nuclear transfer (SCNT) to transfer the genetically modified genome to a suitable cell, e.g., an enucleated oocyte, and gestating the modified cell (e.g., the modified oocyte) in a non-human animal under suitable conditions to form an embryo.

Methods for modifying the germline genome of a non-human animal (e.g., a pig, cow, rodent, chicken, etc. genome) include, e.g., employing a zinc finger nuclease (ZFN), a transcription activator-like effector nuclease (TALEN), or a Cas protein (i.e., a CRISPR/Cas system) to include an exogenous HSD17B13 gene. Guidance for methods for modifying the germline genome of a non-human animal can be found in, e.g., U.S. Pat. No. 9,738,897, and U.S. Patent Application Publication Nos. US 2016/0145646 (published May 26, 2016) and US 2016/0177339 (published Jun. 23, 2016).

In some embodiments, a non-human animal as described herein is a mammal. In some embodiments, a non-human animal as described herein is a small mammal, e.g., of the superfamily Dipodoidea or Muroidea. In some embodiments, a genetically modified animal as described herein is a rodent. In some embodiments, a rodent as described herein is selected from a mouse, a rat, and a hamster. In some embodiments, a rodent as described herein is selected from the superfamily Muroidea. In some embodiments, a genetically modified animal as described herein is from a family selected from Calomyscidae (e.g., mouse-like hamsters), Cricetidae (e.g., hamster, New World rats and mice, voles), Muridae (true mice and rats, gerbils, spiny mice, crested rats), Nesomyidae (climbing mice, rock mice, white-tailed rats, Malagasy rats and mice), Platacanthomyidae (e.g., spiny dormice), and Spalacidae (e.g., mole rates, bamboo rats, and zokors). In some certain embodiments, a genetically modified rodent as described herein is selected from a true mouse or rat (family Muridae), a gerbil, a spiny mouse, and a crested rat. In some certain embodiments, a genetically modified mouse as described herein is from a member of the family Muridae. In some embodiment, a non-human animal as described herein is a rodent. In some certain embodiments, a rodent as described herein is selected from a mouse and a rat. In some embodiments, a non-human animal as described herein is a mouse.

In some embodiments, a non-human animal as described herein is a rodent that is a mouse of a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/L10Cr, and C57BL/Ola. In some certain embodiments, a mouse as described herein is a 129-strain selected from the group consisting of a strain that is 129P1, 129P2, 129P3, 129X1, 129S1 (e.g., 129S1/SV, 129S1/SvIm), 12952, 12954, 129S5, 129S9/SvEvH, 129/SvJae, 129S6 (129/SvEvTac), 129S7, 12958, 129T1, 129T2 (see, e.g., Festing et al., 1999, Mammalian Genome 10:836; Auerbach, W. et al., 2000, Biotechniques 29(5):1024−1028, 1030, 1032). In some certain embodiments, a genetically modified mouse as described herein is a mix of an aforementioned 129 strain and an aforementioned C57BL/6 strain. In some certain embodiments, a mouse as described herein is a mix of aforementioned 129 strains, or a mix of aforementioned BL/6 strains. In some certain embodiments, a 129 strain of the mix as described herein is a 129S6 (129/SvEvTac) strain. In some embodiments, a mouse as described herein is a BALB strain, e.g., BALB/c strain. In some embodiments, a mouse as described herein is a mix of a BALB strain and another aforementioned strain.

In some embodiments, a non-human animal as described herein is a rat. In some certain embodiments, a rat as described herein is selected from a Wistar rat, an LEA strain, a Sprague Dawley strain, a Fischer strain, F344, F6, and Dark Agouti. In some certain embodiments, a rat strain as described herein is a mix of two or more strains selected from the group consisting of Wistar, LEA, Sprague Dawley, Fischer, F344, F6, and Dark Agouti.

A rat pluripotent and/or totipotent cell can be from any rat strain, including, for example, an ACI rat strain (an inbred strain originally derived from August and Copenhagen strains), a Dark Agouti (DA) rat strain, a Wistar rat strain, a LEA rat strain, a Sprague Dawley (SD) rat strain, or a Fischer rat strain such as Fisher F344 or Fisher F6. Rat pluripotent and/or totipotent cells can also be obtained from a strain derived from a mix of two or more strains recited above. For example, the rat pluripotent and/or totipotent cell can be from a DA strain or an ACI strain. The ACI rat strain is characterized as having black agouti, with white belly and feet and an RTlav1 haplotype. Such strains are available from a variety of sources including Harlan Laboratories. An example of a rat ES cell line from an ACI rat is an ACI.G1 rat ES cell. The DA rat strain is characterized as having an agouti coat and an RTlav1 haplotype. Such rats are available from a variety of sources including Charles River and Harlan Laboratories. Examples of a rat ES cell line from a DA rat are the DA.2B rat ES cell line and the DA.2C rat ES cell line. In some embodiments, the rat pluripotent and/or totipotent cells are from an inbred rat strain (see, e.g., U.S. Patent Application Publication No. 2014-0235933 A1). Guidance for making modifications in a rat genome (e.g., in a rat ES cell) using methods and/or constructs as described herein can be found in, e.g., in U.S. Patent Application Publication Nos. 2014-0310828 and 2017-0204430.

In some embodiments, useful technologies are described in, e.g., U.S. Ser. No. 10/314,297 and can be utilized in accordance with the present disclosure. As those skilled in the art appreciate, many useful technologies are commercially available from various venders and/or service providers.

In some embodiments, the present disclosure provides methods for assessing an agent, e.g., an oligonucleotide, or a composition thereof, comprising administering to an animal, cell or tissue described herein the agent or composition. In some embodiments, an agent or composition is assessed for preventing or treating a condition, disorder or disease. In some embodiments, animals, cells, tissues, e.g., as described in various embodiments herein, are animal models, or cells or tissues, for various conditions, disorders or diseases (e.g., comprising mutations associated with various conditions, disorders or diseases, and/or cells, tissues, organs, etc., associated with or of various conditions, disorders or diseases) that are engineered to comprise and/or express a polynucleotide whose sequence encodes an HSD17B13 polypeptide or a characteristic portion thereof. In some embodiments, animals may be provided by breeding (e.g., IVF, natural breeding, etc.) an animal that are model animals for various conditions, disorders or diseases but are not engineered to comprise and/or express a polynucleotide whose sequence encodes an HSD17B13 polypeptide or a characteristic portion thereof with animals that are engineered to comprise and/or express a polynucleotide whose sequence encodes an HSD17B13 polypeptide or a characteristic portion thereof. In some embodiments, cells or tissues may be provided by introducing into cells or tissues a polynucleotide whose sequence encodes an HSD17B13 polypeptide or a characteristic portion thereof. In some embodiments, the present disclosure provides a method for preventing or treating a condition, disorder or disease, comprising administering to a subject an effective amount of an agent or a compositions thereof, wherein the agent or composition is assessed in an animal provided herein (e.g., an animal engineered to comprise an HSD17B13 polypeptide or a characteristic portion thereof, an animal engineered to comprise and/or express a polynucleotide whose sequence encodes an HSD17B13 polypeptide or a characteristic portion thereof, a model animal for a condition, disorder or disease which is engineered to comprise an HSD17B13 polypeptide or a characteristic portion thereof, a model animal for a condition, disorder or disease engineered to comprise and/or express a polynucleotide whose sequence encodes an HSD17B13 polypeptide or a characteristic portion thereof). In some embodiments, the present disclosure provides a method for preventing or treating a condition, disorder or disease, comprising administering to a subject an effective amount of an agent or a compositions thereof, wherein the agent or composition is assessed in a cell or tissue provided herein. In some embodiments, an animal is a non-human animal. In some embodiments, cells are non-human animal cells. In some embodiments, tissues are non-human animal tissues. In some embodiments, a non-human animal is a rodent. In some embodiments, a non-human animal is a mouse. In some embodiments, a non-human animal is a rat. In some embodiments, a non-human animal is a non-human primate.

As appreciated by those skilled in the art, in some embodiments, animals can be heterozygous with respect to one or more or all sequences. In some embodiments, animals are homozygous with respect to one or more or all sequences. In some embodiments, animals are hemizygous with respect to one or more or all engineered sequences. In some embodiments, animals are homozygous with respect to one or more sequences, and heterozygous with respect to one or more sequences. In some embodiments, animals are heterozygous with respect to a polynucleotide whose sequence encodes an HSD17B13 polypeptide or a characteristic portion thereof. In some embodiments, animals are homozygous with respect to a polynucleotide whose sequence encodes an HSD17B13 polypeptide or a characteristic portion thereof. In some embodiments, animals are homozygous wild-type with respect to a loci encoding a polynucleotide whose sequence encodes an HSD17B13 polypeptide or a characteristic portion thereof (e.g., do not express an exogenous HSD17B13 polypeptide or a characteristic portion thereof), and may act as a relative control. In some embodiments, certain animals are heterozygous with respect to one or more polynucleotide sequences associated with various condition, disorder or diseases, and are heterozygous with respect to a polynucleotide whose sequence encodes an HSD17B13 polypeptide or a characteristic portion thereof. In some embodiments, certain animals are homozygous with respect to one or more polynucleotide sequences associated with various conditions, disorders, or diseases, and are heterozygous with respect to a polynucleotide whose sequence encodes an HSD17B13 polypeptide or a characteristic portion thereof. In some embodiments, certain animals are heterozygous with respect to one or more polynucleotide sequences associated with various conditions, disorders, or diseases, and are homozygous with respect to a polynucleotide whose sequence encodes an HSD17B13 polypeptide or a characteristic portion thereof. In some embodiments, certain animals are homozygous with respect to one or more polynucleotide sequences associated with various conditions, disorders, or diseases, and are homozygous with respect to a polynucleotide whose sequence encodes an HSD17B13 polypeptide or a characteristic portion thereof. Cells or tissues may be similarly heterozygous, hemizygous and/or homozygous with respect to various sequences.

In some embodiments, the present disclosure provides methods comprising: 1) assessing an agent or a composition thereof, comprising contacting the agent or a composition thereof with a provided cell or tissue associated with or of a condition, disorder, or disease, and 2) administering to a subject suffering from or susceptible to a condition, disorder, or disease an effective amount of an agent or composition thereof. In some embodiments, the present disclosure provides methods comprising: 1) assessing an agent or a composition thereof, comprising administering the agent or a composition thereof to a provided animal which is an animal model of a condition, disorder, or disease, and 2) administering to a subject suffering from or susceptible to a condition, disorder, or disease an effective amount of an agent or composition thereof. In some embodiments, as described herein, a cell, tissue or animal is engineered to comprise an HSD17B13 polypeptide or a characteristic portion thereof. In some embodiments, a cell, tissue or animal is engineered to comprise and/or express a polynucleotide whose sequence encodes an HSD17B13 polypeptide or a characteristic portion thereof. In some embodiments, a cell, tissue or animal further comprises a nucleotide sequence (e.g., a mutation) associated with a condition, disorder, or disease. In some embodiments, an animal is a rodent, e.g., a mouse, a rat, etc. In some embodiments, a cell or tissue is of a rodent, e.g., a mouse, a rat, etc. In some embodiments, a cell is a germline cell. In some embodiments, a fraction of and not all cells, e.g., cells of particular cell types or tissues or location, of a population of cells, a tissue or an animal comprise a nucleotide sequence (e.g., a mutation) associated with a condition, disorder, or disease, and such fraction of cells are engineered to comprise an HSD17B13 polypeptide or a characteristic portion thereof or engineered to comprise and/or express a polynucleotide whose sequence encodes an HSD17B13 polypeptide or a characteristic portion thereof. Those skilled in the art appreciate that various technologies are available for optionally controlled introduction and/or expression of a nucleotide sequence in various cells, tissues, or organs and can be utilized in accordance with the present disclosure. In some embodiments, as described herein, a cell, tissue or animal comprises a polynucleotide whose sequence encodes an HSD17B13 polypeptide or a characteristic portion thereof in a genome, in some embodiments, in a germline genome. In some embodiments, as described herein, a cell, tissue or animal comprises a nucleotide sequence (e.g., a mutation) associated with a condition, disorder or disease in a genome, in some embodiments, in a germline genome.

### Methods of Using Provided Non-Human Animals, Cells or Tissues

Non-human animals (e.g., rodents, e.g., rats or mice), non-human (e.g., rodent, e.g., rat or mouse) cells and non-human (e.g., rodent, e.g., rat or mouse) tissues as described herein can be used as a platform for the development of therapeutic agents, e.g., oligonucleotides. In particular, non-human animals, non-human cells and non-human tissues as described herein represent a particularly advantageous platform for the identification and characterization of agents, e.g., ds oligonucleotides targeting HSD17B13 can be useful for treatment of conditions, disorders, or diseases associated with expression of HSD17B13, e.g., nonalcoholic fatty liver disease (NAFLD), nonalcoholic steatohepatitis (NASH), alcoholic steatohepatitis (ASH), alcoholic liver disease, nonalcoholic liver disease, alcoholic cirrhosis, nonalcoholic cirrhosis, steatohepatitis, hepatic steatosis, hepatoceullar carcinoma, HCV hepatitis, chronic hepatitis, hereditary hemochromatosis, primary sclerosing cholangitis, drug induced liver injury, or hepatocellular necrosis.

In some embodiments, the present disclosure provides that non-human animals (e.g., rodents, e.g., rats or mice), non-human (e.g., rodent, e.g., rat or mouse) cells and non-human (e.g., rodent, e.g., rat or mouse) tissues described herein can be used in methods characterizing/assessing various agents, e.g., ds oligonucleotides targeting HSD17B13 useful for treatment of conditions, disorders, or diseases associated with expression of HSD17B13. In some embodiments, a composition is an oligonucleotide composition. In some embodiments, oligonucleotides comprise various modifications, e.g., base, sugar, internucleotidic linkage modifications, etc. In some embodiments, linkage phosphorus in a modified internucleotidic linkage, e.g., a phosphorothioate internucleotidic linkage, is chiral (as appreciated by those skilled in the art, natural phosphate linkages commonly found in natural DNA and RNA molecules are achiral). In some embodiments, for various biological or therapeutic uses oligonucleotides comprise extensive modifications, and in some cases, contain no natural RNA sugars for, e.g., improved stability. In some embodiments, a composition is a stereorandom oligonucleotide composition. In some embodiments, a composition is a chirally controlled oligonucleotide composition, wherein one or more or all chiral linkage phosphorus are independently chirally controlled.

Utilizing Non-Human Animals for Agent Assessment In vivo

In some embodiments, non-human animals (e.g., rodents, e.g., rats or mice), non-human (e.g., rodent, e.g., rat or mouse) cells and non-human (e.g., rodent, e.g., rat or mouse) tissues as described herein may be employed for characterizing an oligonucleotide in vivo, wherein the expression of exogenous HSD17B13 gene in said non-human animal provides an improved characterization platform when compared to a WT non-human animal (e.g., rodents, e.g., rats or mice).

In some embodiments, a non-human animal (e.g., genetically modified rodent, e.g., genetically modified rat or mouse) as described herein is treated (e.g., injected) with a ds oligonucleotide of interest under conditions and for a time sufficient that the ds oligonucleotide can target the exogenous HSD17B13. Sequences of RNA molecules (e.g., HSD17B13 sequences) are isolated and/or identified from the treated non-human animal (or one or more cells, for example, one or more B cells) and characterized using various assays measuring, for example, affinity, specificity, editing levels, transcript stability, translational efficiency, protein binding partners, nuclear localization, etc. In various embodiments, oligonucleotides characterized using non-human animals, non-human cells and/or non-human tissues as described herein comprise one or more regions that facilitate targeting of an HSD17B13 target.

In some embodiments, a non-human animal (e.g., genetically modified rodent, e.g., genetically modified rat or mouse) as described herein is treated with a ds oligonucleotide of interest and the effects of said ds oligonucleotide in specific tissues are monitored and/or assessed.

In some embodiments, non-human (e.g., rodent, e.g., rat or mouse) cells as described herein comprising a transgenic HSD17B13 locus may be employed for methods of characterizing potentially therapeutically efficacious oligonucleotides, the method comprising characterization in cells derived from the HSD17B13 transgenic mouse. In some embodiments, such cells may be of any cell lineage and/or type of interest known in the art. In some embodiments, such cells may be but are not limited to: primary mouse hepatocytes, epidermal cells, epithelial cells, cortical neurons, sensory neurons, effector neurons, hormone-secreting cells, exocrine secretory epithelial cells, barrier cells, cardiomyocytes, leukocytes, lymphocytes, B cells, T cells, Bone Marrow cells, osteoblasts, chondrocytes, chondroblasts, adipocytes, cardiac muscle cells, muscle cells, fibroblasts, germ cells, nurse cells, kidney cells and/or an induced stem cell or product thereof derived from any of the aforementioned cells.

Non-human animals (e.g., rodents, e.g., rats or mice), non-human (e.g., rodent, e.g., rat or mouse) cells and non-human (e.g., rodent, e.g., rat or mouse) tissues as described herein can be employed for identifying ds oligonucleotides targeting HSD17B13, which can be useful for treatment of conditions, disorders, or diseases associated with expression of HSD17B13, e.g., nonalcoholic fatty liver disease (NAFLD), nonalcoholic steatohepatitis (NASH), alcoholic steatohepatitis (ASH), alcoholic liver disease, nonalcoholic liver disease, alcoholic cirrhosis, nonalcoholic cirrhosis, steatohepatitis, hepatic steatosis, hepatoceullar carcinoma, HCV hepatitis, chronic hepatitis, hereditary hemochromatosis, primary sclerosing cholangitis, drug induced liver injury, or hepatocellular necrosis.

Non-human animals (e.g., rodents, e.g., rats or mice), non-human (e.g., rodent, e.g., rat or mouse) cells and non-human (e.g., rodent, e.g., rat or mouse) tissues as described herein provide an improved in vivo system and source of biological materials (e.g., cells, nucleotides, polypeptides, protein complexes) for producing and characterizing oligonucleotides and/or polynucleotides that are useful for a variety of assays. In various embodiments, non-human animals, non-human cells and non-human tissues as described herein are used to develop therapeutics that target an RNA of interest (e.g., a RNA molecule known to function in a disease associated pathway) and/or modulate one or more activities associated with said RNA molecules of interest and/or modulate interactions of said RNA molecule of interest with other potential binding partners (e.g., any regulatory machinery that can act on intracellular RNA molecules, e.g., proteins and/or RNA species involved in translation, proteins and/or RNA species involved in innate immunity, proteins and/or RNA species involved in RNA interference, etc.).

In various embodiments, non-human animals (e.g., rodents, e.g., rats or mice), non-human (e.g., rodent, e.g., rat or mouse) cells and non-human (e.g., rodent, e.g., rat or mouse) tissues as described herein are used to determine the pharmacokinetic profiles of one or more ds oligonucleotide candidates. In various embodiments, one or more non-human animals, non-human cells and non-human tissues as described herein and one or more control or reference non-human animals, non-human cells and non-human tissues are each exposed to one or more agents, e.g., ds oligonucleotides at various doses (e.g., less than 0.1 mg/kg, 0.1 mg/kg, 0.2 mg/kg, 0.3 mg/kg, 0.4 mg/kg, 0.5 mg/kg, 1 mg/kg, 2 mg/kg, 3 mg/kg, 4 mg/kg, 5 mg/mg, 7.5 mg/kg, 10 mg/kg, 15 mg/kg, 20 mg/kg, 25 mg/kg, 30 mg/kg, 40 mg/kg, or 50 mg/kg or more). In some embodiments, oligonucleotides may be dosed to non-human animals at rates that vary as a function of gender, for example, in some embodiments a male animal may receive a higher dose than a comparable female animal, while in other embodiments, a female animal may receive a higher dose than a comparable male animal. In some embodiments, candidate therapeutic oligonucleotides may be dosed to non-human animals as described herein via any desired route of administration including parenteral and non-parenteral routes of administration. Parenteral routes include, e.g., intravenous, intra-arterial, intraportal, intramuscular, subcutaneous, intraperitoneal, intraspinal, intrathecal, intracerebroventricular, intracranial, intrapleural or other routes of injection. In some embodiments, administration may be non-parenteral, in some embodiments non-parenteral routes include, e.g., oral, nasal, transdermal, pulmonary, rectal, buccal, vaginal, ocular. In some embodiments, administration may also be by continuous infusion, local administration, sustained release from implants (gels, membranes or the like), and/or intravenous injection. In some embodiments, biological tissue (e.g., organs, blood, cells, secretions etc.) is isolated from non-human animals (humanized and control) at various time points (e.g., 0 hr, 6 hr, 1 day, 2 days, 3 days, 4 days, 5 days, 6 days, 7 days, 8 days, 9 days, 10 days, 11 days, or up to 30 or more days). Various assays may be performed to determine the pharmacokinetic profiles of administered candidate therapeutic oligonucleotides using samples obtained from non-human animals, non-human cells and non-human tissues as described herein including, but not limited to, editing levels, transcript levels, translational levels etc.

In various embodiments, non-human animals (e.g., rodents, e.g., rats or mice), non-human (e.g., rodent, e.g., rat or mouse) cells and non-human (e.g., rodent, e.g., rat or mouse) tissues as described herein are used to measure the therapeutic effect of blocking or modulating the activity of an RNA molecule of interest and the effect on gene expression as a result of cellular changes thereof.

Cells from provided non-human animals (e.g., rodents, e.g., rats or mice) can be isolated and used on an ad hoc basis, or can be maintained in culture for many generations. In various embodiments, cells from a provided non-human animal are immortalized (e.g., via use of a virus) and maintained in culture indefinitely (e.g., in serial cultures).

In some embodiments, a non-human (e.g., rodent, e.g., rat or mouse) cell is a non-human lymphocyte. In some embodiments, a non-human cell is selected from a B cell, dendritic cell, macrophage, monocyte and a T cell. In some embodiments, a non-human cell is an immature B cell, a mature naïve B cell, an activated B cell, a memory B cell, and/or a plasma cell.

In some embodiments, a non-human (e.g., rodent, e.g., rat or mouse) cell is a non-human embryonic stem (ES) cell. In some embodiments, a non-human ES cell is a rodent ES cell. In some certain embodiments, a rodent ES cell is a mouse ES cell and is from a 129 strain, C57BL strain, BALB/c or a mixture thereof. In some certain embodiments, a rodent embryonic stem cell is a mouse embryonic stem cell and is a mixture of 129 and C57BL strains. In some certain embodiments, a rodent embryonic stem cell is a mouse embryonic stem cell and is a mixture of 129, C57BL and BALB/c strains.

In some embodiments, use of a non-human (e.g., rodent, e.g., rat or mouse) ES cell as described herein to make a non-human animal is provided. In some certain embodiments, a non-human ES cell is a mouse ES cell and is used to make a mouse comprising exogenous HSD17B13 as described herein. In some certain embodiments, a non-human ES cell is a rat ES cell and is used to make a rat comprising exogenous HSD17B13 as described herein. In some embodiments, a non-human (e.g., rodent, e.g., rat or mouse) tissue is selected from but not limited to adipose, bladder, brain, breast, bone marrow, eye, heart, intestine, kidney, liver, lung, lymph node, muscle, pancreas, plasma, serum, skin, spleen, stomach, thymus, testis, ovum, and/or a combination thereof.

In some embodiments, an immortalized cell made, generated, produced or obtained from an isolated non-human cell or tissue as described herein is provided.

In some embodiments, a non-human (e.g., rodent, e.g., rat or mouse) embryo made, generated, produced, or obtained from a non-human ES cell as described herein is provided. In some certain embodiments, a non-human embryo is a rodent embryo; in some embodiments, a mouse embryo; in some embodiments, a rat embryo.

In some embodiments, a non-human animal (e.g., rodent, e.g., rat or mouse), non-human (e.g., rodent, e.g., rat or mouse) cell or non-human (e.g., rodent, e.g., rat or mouse) tissue as described herein is provided for use in the manufacture and/or development of a drug (e.g., a ds oligonucleotide or fragment thereof) for therapy or diagnosis.

In some embodiments, a non-human animal (e.g., rodent, e.g., rat or mouse), non-human (e.g., rodent, e.g., rat or mouse) cell or non-human (e.g., rodent, e.g., rat or mouse) tissue as described herein is provided for use in the manufacture and/or development of a medicament for the treatment, prevention or amelioration of a disease, disorder or condition. In some embodiments, use of a non-human animal (e.g., rodent, e.g., rat or mouse), non-human (e.g., rodent, e.g., rat or mouse) cell or non-human (e.g., rodent, e.g., rat or mouse) tissue as described herein in the manufacture and/or development of a drug or vaccine for use in medicine, such as use as a medicament, is provided.

### Kits

The present disclosure further provides a pack or kit comprising one or more containers filled with at least non-human cell, protein (single or complex (e.g., an antibody or fragment thereof)), DNA fragment, targeting vector, or any combination thereof, as described herein. Kits may be used in any applicable method (e.g., a research method). Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, and/or (c) a contract that governs the transfer of materials and/or biological products (e.g., a non-human animal or non-human cell as described herein) between two or more entities and combinations thereof.

In some embodiments, a kit comprising a non-human cell, non-human tissue, immortalized cell, non-human ES cell, or non-human embryo as described herein is provided. In some embodiments, a kit comprising an amino acid from a non-human animal, non-human cell, non-human tissue, immortalized cell, non-human ES cell, or non-human embryo as described herein is provided. In some embodiments, a kit comprising a nucleic acid (e.g., a nucleic acid encoding a human HSD17B13 sequence described herein) from a non-human animal, non-human cell, non-human tissue, immortalized cell, non-human ES cell, or non-human embryo as described herein is provided. In some embodiments, a kit comprising a sequence (amino acid and/or nucleic acid sequence) identified from a non-human animal, non-human cell, non-human tissue, immortalized cell, non-human ES cell, or non-human embryo as described herein is provided.

In some embodiments, a kit as described herein for use in the manufacture and/or development of a drug (e.g., an oligonucleotide) for therapy or diagnosis is provided.

In some embodiments, a kit as described herein for use in the manufacture and/or development of a drug (e.g., an oligonucleotide) for the treatment, prevention or amelioration of a disease, disorder or condition is provided.

Other features of certain embodiments will become apparent in the course of the following descriptions of exemplary embodiments, which are given for illustration and are not intended to be limiting thereof.

### EXEMPLIFICATION

Certain examples of provided technologies (compounds (oligonucleotides, reagents, etc.), compositions, methods (methods of preparation, use, assessment, etc.), etc.) were described below.

### Example 1. Oligonucleotide Synthesis

Various technologies for preparing oligonucleotides and oligonucleotide compositions (both stereorandom and chirally controlled) are known and can be utilized in accordance with the present disclosure, including, for example, those in U.S. Pat. Nos. 9,394,333, 9,744,183, 9,605,019, 9,598,458, 9,982,257, U.S. Ser. No. 10/160,969, U.S. Ser. No. 10/479,995, US 2020/0056173, US 2018/0216107, US 2019/0127733, U.S. Ser. No. 10/450,568, US 2019/0077817, US 2019/0249173, US 2019/0375774, WO 2018/223056, WO 2018/223073, WO 2018/223081, WO 2018/237194, WO 2019/032607, WO 2019/055951, WO 2019/075357, WO 2019/200185, WO 2019/217784, WO 2019/032612, and/or WO 2020/191252, the methods and reagents of each of which are incorporated herein by reference.

### Abbreviations

1λ reagent: TEA-3HF:TEA:H2O: DMSO=5.0:1.8:15.5:77.7 (v/v/v/v)
ADIH: 2-azido-1,3-dimethylimidazolium hexafluorophosphate
CMIMT: N-cyanomethylimidazolium triflate
CPG: controlled pore glass
DCM: dichloromethane, CH2Cl2
DIPEA: diisopropylethylamine
DMSO: dimethylsulfoxide
DMTr: 4,4′-dimethoxytrityl
GalNAc: N-acetylgalactosamine
HF: hydrogen fluoride
HATU: 1-[bis(dimethylamino)methylene]-1H-1,2,3-triazolo[4,5-b]pyridinium 3-oxide hexafluorophosphate
IBN: isobutyronitrile
MeCN: acetonitrile
MeIm: N-methylimidazole
TCA: trichloroacetic acid
TEA: triethylamine
XH: xanthane hydride

The HSD17B13 sequences, both guide and passenger strands are stereodefined oligonucleotides, with a mixed PS/PO/PN backbone. Respective stereorandom sequences were synthesized as positive controls. The number of PS/PO/PN linkages varies from sequences to sequence. For synthesizing PO, PS and PN linkages different amidites and synthetic cycles are used. Example given, for synthesizing an oligonucleotide, defined number of phosphodiester (PO) linkage were formed by cyanoethyl protected amidite using oxidation step and stereorandom phosphorothioate (PS) linkage were formed by cyanoethyl protected amidite using sulfurization step. Defined number of chiral phosphorothioate (PS)(Sp and Rp linkages), formed by DPSE protected chiral amidites using sulfurization step and defined number of chiral phosphoroamidates (PN) (Sp and Rp) linkages formed by PSM chiral auxiliary amidites using imidation step. All the sequences carry various modifications, especially 2′-modifications including 2′-OMe and 2′-F-modified nucleotides. Passenger strand oligonucleotides have 5′-GalNAc modifications (tri-antennary GalNAc moiety) at 5′-end. For introduction of GalNAc moiety at 5′-end, sequences were synthesized by coupling with C-6 amino modifier as the last coupling cycle and after purification and desalting are conjugated with tri-antennary GalNAc to make respective conjugate. For example, WV-42597 was synthesized first and then conjugated to make WV-42589. The synthesis process is described in detail here.

| | |
|---|---|
| WV-42597 | Passenger sequence with 5′-amino linker |
| WV-42589 | GalNac-conjugate of WV-42597 |

Procedure for the synthesis of WV-42597:

Synthesis of WV-42597 was performed on AKTA OP100 synthesizer (GE healthcare) using a 3.5 cm diameter SS column on a 400 μmol scale using a CPG support (loading 72 μmol/g). The process consisted of five steps: detritylation, coupling, capping 1, oxidation/sulfurization/imidation and capping 2.

Detritylation step: detritylation was performed using 3% DCA in toluene with a UV watch command set at 436 nm. Following detritylation step, the CPG support was subjected to wash cycle using acetonitrile for 2CV.

Coupling step: DPSE and PSM chiral amidites were prepared at 0.2M conc. (in ACN or 20% IBN in ACN). The amidites were mixed in-line with CMIMT activator (0.5M in acetonitrile) at a ratio of 5.83 prior to addition to the column. The coupling mixture was recycled for 10 minutes to maximize the coupling efficiency followed by column wash with 2CV of ACN.

Cyanoethyl amidites were prepared at 0.2M conc. (in ACN or 20% IBN in ACN). The amidites were mixed in-line with ETT activator (0.5M in acetonitrile) at a ratio of 4.07 prior to addition to the column. The coupling mixture was recycled for 10 minutes to maximize the coupling efficiency followed by column wash with 2CV of ACN.

Cap 1 step: For stereodefined couplings, the column was then treated with Capping 1 solution (acetic anhydride, lutidine, ACN) for 1 CV in 2 minutes to acetylate the chiral auxiliary amine. Following this step, the column was washed with 1.5 CV of acetonitrile. For stereorandom coupling Cap 1 step was not performed.

### Sulfurization/Imidation/Oxidation Step:

Sulfurization was performed with 0.1 M xanthane hydride in pyridine/acetonitrile (1.2 equivalent) with a contact time of 6 minute followed by 2CV wash step.

Imidation step was performed with 0.3 M ADIH reagent in acetonitrile with 18 equivalent and 15 min contact time followed by 2CV wash step.

Oxidation step was performed using oxidation reagent (50 mM 12/pyridine-H2O (9:1, v/v) ) 3.5 eq. 2.5 minute followed by 2CV acetonitrile wash.

Capping 2 step: Capping 2 step was performed using Capping A and Capping B reagents mixed inline (1:1) followed by a 2 CV ACN wash.

### General Procedure for the C&D Conditions:

After completion of the synthesis, the CPG support was finally treated with 20% diethylamine/acetonitrile wash step for 5 column volume/15 mins to remove the cyanoethyl protecting groups exclusively from the phosphate backbone followed by ACN wash cycle. The CPG solid support was dried and transferred into pressure vessel.

The following cleavage and deprotection protocol is described for (WV-42597): The DPSE protecting groups on SERPINA1-1159 were removed by treating the oligo bound support with desilylation reagent at a ratio of per μmole support/100 μL desilylation reagent. The desilylation reagent was made by mixing DMSO:water:TEA:TEA.3HF in ratio of 7.33:1.47:0.7:0.5. The CPG support was incubated in presence with desilylation reagent for 3 hours at 27° C. in an incubator shaker. After that conc. Ammonia was added at a ratio of per μmole support/200 μL of conc. ammonia to remove PSM auxiliaries, the protecting groups on nucleobases and the oligonucleotide from the CPG support. The mixture was incubated and shaken for 24 hours at 37° C. The mixture was cooled and filtered using 0.2-0.45 micron filter and the CPG support was rinsed three times to collect all the desired material as filtrate. The filtrate containing crude oligonucleotide was analyzed by RP-UPLC and quantitation was done using a Nanodrop One Spectrophotometer (Thermo Scientific) and a yield of 110,000 OD/μmole was obtained.

Purification and desalting of WV-42597: The crude WV-42597 was loaded on to Waters AP-2 glass column (2.0 cm×20 cm) packed with Source 15Q (Cytiva). Purification was performed on an AKTA150 Pure (GE healthcare) using following buffers: (Buffer A: 20 mM NaOH, 20% Acetonitrile v/v) (Buffer B: 20 mM NaOH, 2.5M NaCl, 20% Acetonitrile v/v). Desired fractions with full length products in the range of 70-80% were pooled together. The pooled material was then desalted on a 2KD re-generated cellulose membrane followed by lyophilization to obtain SERPINA1-1159 as fluffy white cake ready for conjugation.

Synthesis of WV-42589:

Protocol for GalNAc conjugation:

Precursor material: WV-42597.01 (0.01 denoting the batch number)

Final conjugated material: WV-42589.01

| | Mol. | Equivalent to | |
|---|---|---|---|
| Oligonucleotide/Reagents | Wt. | oligonucleotide | mL |
| WV-42597 | 10050.80 | 1 | — |
| Tri-antennary GalNAc acid | 2006 | 1.8 | — |
| HATU | 382 | 1.4 | — |
| DIEA | 129 | 10 | — |
| Acetonitrile | | — | 4 |

Aqueous oligonucleotide solution

| | Conc. | Total | Total |
|---|---|---|---|
| Oligonucleotide/solvent | (mg/mL) | volume (mL) | mg |
| WV-42597 in WFI water | 50 | 8 | 400 |

The Tri-antennary GalNAc acid, HATU are weighed out in a 50 mL plastic tube and dissolved in anhydrous acetonitrile then DIEA was added into the tube. The resulting mixture was stirred for 10 min at 37° C. The lyophilized WV-42597 was reconstituted in water in a separate tube and the GalNAc mixture was added to the oligonucleotide solution and stirred for 60 min at 37° C. The reaction was monitored by RP-UPLC. Reaction was found to be complete in 1 h. The reaction mixture was concentrated under vacuum to remove the acetonitrile and the resultant GalNAc-conjugated oligonucleotide was treated with conc. ammonia for 2 h at 37° C. The formation of final product was confirmed by mass spectrometry and RP-UPLC. The conjugated material was purified by anion exchange chromatography and desalted using tangential flow filtration (TFF) to obtain the final product (Target mass: 8708.54; Observed mass: 8709.9).

### Example 2. Provided Oligonucleotides and Compositions can Effectively Knockdown HSD17B13 In Vitro

Various Double stranded oligonucleotides targeting HSD17B13 and compositions were designed, constructed, characterized and assessed. As appreciated by those skilled in the art, various technologies can be utilized to assess properties and/or activities of provided oligonucleotides and compositions thereof. Some of such technologies are described in this Example. Those skilled in the art appreciate that many other technologies can be readily utilized in accordance with the present disclosure. As demonstrated herein, double stranded oligonucleotides targeting HSD17B13 and compositions, among other things, can be highly active, e.g., in reducing levels of their target nucleic acids and proteins encoded thereby.

A number of siRNAs were tested in vitro in human or NHP primary hepatocytes at one or a range of concentrations. Example protocol for in vitro determination of siRNA activity: For determination of siRNAs activity, siRNAs at specific concentration were delivered to, either gymnotically or by transfection with lipofectamine, human or NHP primary hepatocytes plated at 96-well plates, with 10,000 (human) or 40,000 (NHP) cells/well. Following 48 hours treatment, total RNA was extracted using SV96 Total RNA Isolation kit (Promega). cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For human HSD17B13 mRNA, the following qPCR assay was utilized: Forward, 5′-CGAAGGGATTCCTTACCTCATC-3′; Reverse, 5′-CCAAGGCCTGAAGTTCTGAT-3′; Probe, 5′ATTGCCGCTGTTGGCTTTCACAG-3′. For NHP HSD17B13, the following qPCR assay was utilized: Thermo Fisher Mf02888851_ml. Human SFRS9 was used as normalizer (Forward, 5′-TGGAATATGCCCTGCGTAAA-3′; Reverse, 5′-TGGTGCTTCTCTCAGGATAAAC-3′, Probe, 5′-TGGATGACACCAAATTCCGCTCTCA-3′. mRNA knockdown levels were calculated as % mRNA remaining relative to mock treatment.

Table 2 shows % human HSD17B13 mRNA remaining (1 nM siRNA treatment by transfection) relative to human SFRS9 control. N=2. N.D.: Not determined.

TABLE 2

| | | 1 nM | | |
|---|---|---|---|---|
| | | % remaining mRNA | % remaining mRNA | |
| Guide | Passenger | (hHSD17B13/hSFRS9)-1 | (hHSD17B13/hSFRS9)-2 | Mean |
| WV-38227 | WV-38419 | 42.72 | 50.68 | 46.70 |
| WV-38228 | WV-38420 | 41.13 | 49.56 | 45.35 |
| WV-38229 | WV-38421 | 42.98 | 50.33 | 46.65 |
| WV-38230 | WV-38422 | 49.68 | 76.01 | 62.84 |
| WV-38231 | WV-38423 | 30.47 | 49.28 | 39.88 |
| WV-38232 | WV-38424 | 27.00 | 45.23 | 36.11 |
| WV-38233 | WV-38425 | 29.74 | 43.48 | 36.61 |
| WV-38234 | WV-38426 | 23.30 | 37.81 | 30.55 |
| WV-38235 | WV-38427 | 28.58 | 37.33 | 32.95 |
| WV-38236 | WV-38428 | 25.12 | 34.05 | 29.58 |
| WV-38237 | WV-38429 | 23.82 | 33.29 | 28.55 |
| WV-38238 | WV-38430 | 32.16 | 33.31 | 32.74 |
| WV-38239 | WV-38431 | 23.85 | 36.70 | 30.28 |
| WV-38240 | WV-38432 | N.D. | 27.72 | 27.72 |
| WV-38241 | WV-38433 | 21.91 | 32.85 | 27.38 |
| WV-38242 | WV-38434 | 14.66 | 34.29 | 24.48 |
| WV-38243 | WV-38435 | 25.81 | 26.50 | 26.15 |
| WV-38244 | WV-38436 | 31.66 | 30.02 | 30.84 |
| WV-38245 | WV-38437 | 30.53 | 43.80 | 37.17 |
| WV-38246 | WV-38438 | 25.63 | 29.07 | 27.35 |
| WV-38247 | WV-38439 | 28.35 | 34.75 | 31.55 |
| WV-38248 | WV-38440 | 27.64 | 34.98 | 31.31 |
| WV-38249 | WV-38441 | 19.98 | 28.55 | 24.27 |
| WV-38250 | WV-38442 | 18.41 | 28.33 | 23.37 |
| WV-38251 | WV-38443 | N.D. | 24.89 | 24.89 |
| WV-38252 | WV-38444 | 21.37 | 37.71 | 29.54 |
| WV-38253 | WV-38445 | 31.64 | 42.02 | 36.83 |
| WV-38254 | WV-38446 | 39.09 | 38.06 | 38.57 |
| WV-38255 | WV-38447 | 41.87 | 51.41 | 46.64 |
| WV-38256 | WV-38448 | 38.18 | 55.01 | 46.60 |
| WV-38257 | WV-38449 | 19.77 | 33.14 | 26.46 |
| WV-38258 | WV-38450 | 19.16 | 27.16 | 23.16 |
| WV-38259 | WV-38451 | 23.90 | 27.85 | 25.87 |
| WV-38260 | WV-38452 | 38.20 | 48.66 | 43.43 |
| WV-38261 | WV-38453 | 36.10 | 44.81 | 40.45 |
| WV-38262 | WV-38454 | 42.68 | 71.28 | 56.98 |
| WV-38263 | WV-38455 | 30.26 | 35.27 | 32.77 |
| WV-38264 | WV-38456 | 30.91 | 44.96 | 37.94 |
| WV-38265 | WV-38457 | 27.30 | 47.03 | 37.17 |
| WV-38266 | WV-38458 | 21.28 | 37.30 | 29.29 |
| WV-38267 | WV-38459 | 25.47 | 32.06 | 28.77 |
| WV-38268 | WV-38460 | 29.94 | 35.08 | 32.51 |
| WV-38269 | WV-38461 | 27.17 | 37.63 | 32.40 |
| WV-38270 | WV-38462 | 26.14 | 36.69 | 31.41 |
| WV-38271 | WV-38463 | 34.57 | 36.74 | 35.66 |
| WV-38272 | WV-38464 | 29.86 | 42.49 | 36.17 |
| WV-38273 | WV-38465 | 34.90 | 64.75 | 49.83 |
| WV-38274 | WV-38466 | 54.06 | 71.75 | 62.91 |
| WV-38275 | WV-38467 | 31.70 | 28.07 | 29.88 |
| WV-38276 | WV-38468 | 46.80 | 45.46 | 46.13 |
| WV-38277 | WV-38469 | 27.82 | 35.92 | 31.87 |
| WV-38278 | WV-38470 | 26.32 | 36.22 | 31.27 |
| WV-38279 | WV-38471 | 13.10 | 22.72 | 17.91 |
| WV-38280 | WV-38472 | 28.22 | 37.22 | 32.72 |
| WV-38281 | WV-38473 | 38.95 | 66.96 | 52.96 |
| WV-38282 | WV-38474 | 11.88 | 23.94 | 17.91 |
| WV-38283 | WV-38475 | 20.27 | 30.63 | 25.45 |
| WV-38284 | WV-38476 | 15.58 | 20.32 | 17.95 |
| WV-38285 | WV-38477 | 36.99 | 39.75 | 38.37 |
| WV-38286 | WV-38478 | 26.26 | 27.60 | 26.93 |
| WV-38287 | WV-38479 | 34.93 | 45.38 | 40.16 |
| WV-38288 | WV-38480 | 54.42 | 51.81 | 53.12 |
| WV-38289 | WV-38481 | 27.12 | 33.46 | 30.29 |
| WV-38290 | WV-38482 | 19.72 | 26.55 | 23.13 |
| WV-38291 | WV-38483 | 46.37 | 35.76 | 41.06 |
| WV-38292 | WV-38484 | 21.15 | 24.23 | 22.69 |
| WV-38293 | WV-38485 | 22.68 | 23.83 | 23.25 |
| WV-38294 | WV-38486 | 14.78 | 24.32 | 19.55 |
| WV-38295 | WV-38487 | 51.54 | 61.96 | 56.75 |
| WV-38296 | WV-38488 | N.D. | 24.20 | 24.20 |
| WV-38297 | WV-38489 | 12.91 | 32.26 | 22.58 |
| WV-38298 | WV-38490 | 17.32 | 25.72 | 21.52 |
| WV-38299 | WV-38491 | 30.01 | 23.62 | 26.82 |
| WV-38300 | WV-38492 | 12.83 | 23.31 | 18.07 |
| WV-38301 | WV-38493 | 26.64 | 25.33 | 25.99 |
| WV-38302 | WV-38494 | 22.76 | 28.27 | 25.52 |
| WV-38303 | WV-38495 | 41.95 | 56.77 | 49.36 |
| WV-38304 | WV-38496 | 28.02 | 37.73 | 32.87 |
| WV-38305 | WV-38497 | 13.76 | 15.39 | 14.57 |
| WV-38306 | WV-38498 | 18.54 | 27.69 | 23.11 |
| WV-38307 | WV-38499 | 45.71 | 54.13 | 49.92 |
| WV-38308 | WV-38500 | 20.18 | 25.56 | 22.87 |
| WV-38309 | WV-38501 | 45.03 | 45.88 | 45.45 |
| WV-38310 | WV-38502 | 52.42 | 51.80 | 52.11 |
| WV-38311 | WV-38503 | 56.26 | 98.83 | 77.55 |
| WV-38312 | WV-38504 | 14.73 | 15.17 | 14.95 |
| WV-38313 | WV-38505 | 12.56 | 16.94 | 14.75 |
| WV-38314 | WV-38506 | 36.76 | 45.06 | 40.91 |
| WV-38315 | WV-38507 | 25.86 | 36.26 | 31.06 |
| WV-38316 | WV-38508 | 21.24 | 21.06 | 21.15 |
| WV-38317 | WV-38509 | 11.75 | 11.05 | 11.40 |
| WV-38318 | WV-38510 | 10.40 | 10.10 | 10.25 |
| WV-38319 | WV-38511 | 9.71 | 13.73 | 11.72 |
| WV-38320 | WV-38512 | 26.27 | 35.17 | 30.72 |
| WV-38321 | WV-38513 | 21.75 | 25.22 | 23.48 |
| WV-38322 | WV-38514 | 13.89 | 24.37 | 19.13 |
| WV-38323 | WV-38515 | 32.58 | 37.91 | 35.25 |
| WV-38324 | WV-38516 | 11.46 | 13.14 | 12.30 |
| WV-38325 | WV-38517 | 11.03 | 15.03 | 13.03 |
| WV-38326 | WV-38518 | 92.94 | 101.60 | 97.27 |
| WV-38327 | WV-38519 | 12.18 | 19.20 | 15.69 |
| WV-38328 | WV-38520 | 75.25 | 118.90 | 97.07 |
| WV-38329 | WV-38521 | 14.21 | 26.82 | 20.51 |
| WV-38330 | WV-38522 | 20.56 | 28.78 | 24.67 |
| WV-38331 | WV-38523 | 60.93 | 90.04 | 75.49 |
| WV-38332 | WV-38524 | 14.90 | 19.29 | 17.10 |
| WV-38333 | WV-38525 | 27.12 | 27.95 | 27.53 |
| WV-38335 | WV-38527 | 33.54 | 39.11 | 36.32 |
| WV-38336 | WV-38528 | 11.54 | 18.98 | 15.26 |
| WV-38337 | WV-38529 | 13.53 | 16.31 | 14.92 |
| WV-38338 | WV-38530 | 13.41 | N.D. | 13.41 |
| WV-38339 | WV-38531 | 14.20 | 22.43 | 18.32 |
| WV-38340 | WV-38532 | 16.68 | 16.94 | 16.81 |
| WV-38341 | WV-38533 | 40.47 | 44.93 | 42.70 |
| WV-38342 | WV-38534 | 13.07 | 17.35 | 15.21 |
| WV-38343 | WV-38535 | 22.66 | 27.40 | 25.03 |
| WV-38344 | WV-38536 | 27.14 | 36.79 | 31.96 |
| WV-38345 | WV-38537 | 15.79 | 18.49 | 17.14 |
| WV-38346 | WV-38538 | 10.89 | 19.46 | 15.18 |
| WV-38347 | WV-38539 | 16.17 | 33.36 | 24.77 |
| WV-38348 | WV-38540 | 15.75 | 15.52 | 15.63 |
| WV-38349 | WV-38541 | 30.37 | 37.99 | 34.18 |
| WV-38350 | WV-38542 | 24.82 | 25.20 | 25.01 |
| WV-38351 | WV-38543 | 13.72 | 23.55 | 18.64 |
| WV-38352 | WV-38544 | 13.91 | 19.32 | 16.61 |
| WV-38353 | WV-38545 | 19.14 | 24.29 | 21.71 |
| WV-38354 | WV-38546 | 10.65 | 15.26 | 12.96 |
| WV-38355 | WV-38547 | 15.23 | 24.34 | 19.78 |
| WV-38356 | WV-38548 | 16.07 | 19.68 | 17.87 |
| WV-38357 | WV-38549 | 12.77 | 16.77 | 14.77 |
| WV-38358 | WV-38550 | 25.13 | 21.23 | 23.18 |
| WV-38359 | WV-38551 | 20.14 | 17.76 | 18.95 |
| WV-38360 | WV-38552 | 14.80 | 18.50 | 16.65 |
| WV-38361 | WV-38553 | 15.84 | 20.50 | 18.17 |
| WV-38362 | WV-38554 | 14.82 | 18.24 | 16.53 |
| WV-38363 | WV-38555 | 20.13 | 28.44 | 24.28 |
| WV-38364 | WV-38556 | 21.27 | 26.13 | 23.70 |
| WV-38365 | WV-38557 | 15.11 | 14.27 | 14.69 |
| WV-38366 | WV-38558 | 30.22 | 40.16 | 35.19 |
| WV-38367 | WV-38559 | 80.39 | 72.19 | 76.29 |
| WV-38368 | WV-38560 | 24.81 | 36.22 | 30.51 |
| WV-38369 | WV-38561 | 24.38 | 32.37 | 28.37 |
| WV-38370 | WV-38562 | 17.01 | 21.11 | 19.06 |
| WV-38371 | WV-38563 | 26.43 | 30.19 | 28.31 |
| WV-38372 | WV-38564 | 31.47 | 31.45 | 31.46 |
| WV-38373 | WV-38565 | 25.09 | 37.93 | 31.51 |
| WV-38374 | WV-38566 | 38.40 | 37.48 | 37.94 |
| WV-38375 | WV-38567 | 40.97 | 43.58 | 42.27 |
| WV-38376 | WV-38568 | 30.64 | 36.59 | 33.62 |
| WV-38377 | WV-38569 | 35.11 | 44.12 | 39.61 |
| WV-38378 | WV-38570 | 20.51 | 32.38 | 26.44 |
| WV-38379 | WV-38571 | 34.51 | 38.28 | 36.39 |
| WV-38380 | WV-38572 | 27.92 | 36.68 | 32.30 |
| WV-38381 | WV-38573 | 37.05 | 32.05 | 34.55 |
| WV-38382 | WV-38574 | 37.30 | 39.96 | 38.63 |
| WV-38383 | WV-38575 | 33.96 | 41.07 | 37.51 |
| WV-38384 | WV-38576 | 53.55 | 56.98 | 55.26 |
| WV-38385 | WV-38577 | 37.96 | 39.60 | 38.78 |
| WV-38386 | WV-38578 | 32.80 | 51.22 | 42.01 |
| WV-38387 | WV-38579 | 29.55 | 37.28 | 33.41 |
| WV-38388 | WV-38580 | 36.87 | 38.65 | 37.76 |
| WV-38389 | WV-38581 | 44.41 | 46.48 | 45.44 |
| WV-38390 | WV-38582 | 30.15 | 45.19 | 37.67 |
| WV-38391 | WV-38583 | 38.48 | 50.36 | 44.42 |
| WV-38392 | WV-38584 | 30.10 | 27.99 | 29.05 |
| WV-38393 | WV-38585 | 37.50 | 44.21 | 40.85 |
| WV-38394 | WV-38586 | 24.53 | 38.93 | 31.73 |
| WV-38395 | WV-38587 | 34.73 | 42.20 | 38.46 |
| WV-38396 | WV-38588 | 34.91 | 46.74 | 40.82 |
| WV-38397 | WV-38589 | 38.22 | 41.13 | 39.68 |
| WV-38398 | WV-38590 | 130.09 | 101.44 | 115.77 |
| WV-38399 | WV-38591 | 134.17 | 128.47 | 131.32 |
| WV-38400 | WV-38592 | 143.79 | 119.09 | 131.44 |
| WV-38401 | WV-38593 | 119.51 | 113.29 | 116.40 |
| WV-38402 | WV-38594 | 117.65 | 152.89 | 135.27 |
| WV-38403 | WV-38595 | 118.70 | 121.52 | 120.11 |
| WV-38404 | WV-38596 | 24.18 | 22.64 | 23.41 |
| WV-38405 | WV-38597 | 42.49 | 35.75 | 39.12 |
| WV-38406 | WV-38598 | 21.59 | 30.64 | 26.11 |
| WV-38407 | WV-38599 | 28.35 | 26.38 | 27.37 |
| WV-38408 | WV-38600 | 30.62 | 32.31 | 31.46 |
| WV-38409 | WV-38601 | 23.39 | 24.40 | 23.90 |
| WV-38410 | WV-38602 | 29.05 | 29.51 | 29.28 |
| WV-38411 | WV-38603 | 25.27 | 19.65 | 22.46 |
| WV-38412 | WV-38604 | 23.93 | 30.67 | 27.30 |
| WV-38413 | WV-38605 | 29.69 | 31.01 | 30.35 |
| WV-38414 | WV-38606 | 32.96 | 36.26 | 34.61 |
| WV-38415 | WV-38607 | 20.36 | 24.10 | 22.23 |
| WV-38416 | WV-38608 | 29.11 | 30.73 | 29.92 |
| WV-38417 | WV-38609 | 32.51 | 32.68 | 32.59 |
| WV-38418 | WV-38610 | 28.13 | 26.89 | 27.51 |

Table 3 shows % human HSD17B13 mRNA remaining (at 10, 1 and 0.1 μM by gymnotic delivery) relative to human SFRS9 control. N=2. N.D.: Not determined.

TABLE 3

| | | 10 μM | | | 1 μM | | | 0.1 μM | | |
|---|---|---|---|---|---|---|---|---|---|---|
| | | % remaining | % remaining | | % remaining | % remaining | | % remaining | % remaining | |
| | | mRNA | mRNA | | mRNA | mRNA | | mRNA | mRNA | |
| | | (hHSD17B13/ | (hHSD17B13/ | | (hHSD17B13/ | (hHSD17B13/ | | (hHSD17B13/ | (hHSD17B13/ | |
| Guide | Passenger | hSFRS9)-1 | hSFRS9)-2 | Mean | hSFRS9)-1 | hSFRS9)-2 | Mean | hSFRS9)-1 | hSFRS9)-2 | Mean |
| WV- | WV- | N.D. | N.D. | N.D. | 92.55 | 77.58 | 85.06 | 99.21 | 94.50 | 96.85 |
| 38279 | 38471 | | | | | | | | | |
| WV- | WV- | 57.95 | 68.33 | 63.14 | 117.45 | 107.83 | 112.64 | 96.43 | 120.90 | 108.66 |
| 38282 | 38474 | | | | | | | | | |
| WV- | WV- | 65.94 | 56.24 | 61.09 | 93.12 | 90.08 | 91.60 | 109.04 | 107.69 | 108.37 |
| 38284 | 38476 | | | | | | | | | |
| WV- | WV- | 68.80 | 51.61 | 60.20 | 96.00 | 77.64 | 86.82 | 115.23 | 118.13 | 116.68 |
| 38294 | 38486 | | | | | | | | | |
| WV- | WV- | 75.86 | 74.15 | 75.00 | 111.55 | N.D. | 111.55 | 93.35 | 109.88 | 101.61 |
| 38298 | 38490 | | | | | | | | | |
| WV- | WV- | 77.59 | 76.89 | 77.24 | 107.74 | 95.82 | 101.78 | 106.72 | 106.23 | 106.47 |
| 38300 | 38492 | | | | | | | | | |
| WV- | WV- | 52.07 | 39.79 | 45.93 | 94.76 | 97.06 | 95.91 | 107.20 | 96.64 | 101.92 |
| 38305 | 38497 | | | | | | | | | |
| WV- | WV- | N.D. | N.D. | N.D. | 26.12 | 33.09 | 29.60 | 89.89 | 78.89 | 84.39 |
| 38312 | 38504 | | | | | | | | | |
| WV- | WV- | N.D. | 26.52 | 26.52 | 70.62 | 73.99 | 72.31 | 86.08 | 89.09 | 87.58 |
| 38313 | 38505 | | | | | | | | | |
| WV- | WV- | 23.67 | 34.50 | 29.08 | 61.76 | 68.75 | 65.26 | 81.88 | 85.46 | 83.67 |
| 38317 | 38509 | | | | | | | | | |
| WV- | WV- | 16.33 | 16.90 | 16.61 | 32.19 | 53.22 | 42.70 | 62.12 | 60.90 | 61.51 |
| 38318 | 38510 | | | | | | | | | |
| WV- | WV- | N.D. | 23.39 | 23.39 | 87.20 | 54.47 | 70.84 | 80.76 | 94.36 | 87.56 |
| 38319 | 38511 | | | | | | | | | |
| WV- | WV- | N.D. | N.D. | N.D. | 17.48 | 26.91 | 22.19 | 51.98 | 55.74 | 53.86 |
| 38322 | 38514 | | | | | | | | | |
| WV- | WV- | N.D. | N.D. | N.D. | 15.17 | 12.65 | 13.91 | 39.77 | 44.60 | 42.18 |
| 38324 | 38516 | | | | | | | | | |
| WV- | WV- | 14.32 | 14.50 | 14.41 | 36.37 | 37.22 | 36.80 | 54.44 | 54.61 | 54.52 |
| 38325 | 38517 | | | | | | | | | |
| WV- | WV- | N.D. | 5.95 | 5.95 | 20.81 | 21.80 | 21.31 | 55.61 | 56.54 | 56.08 |
| 38327 | 38519 | | | | | | | | | |
| WV- | WV- | 29.47 | 31.41 | 30.44 | 41.61 | 46.86 | 44.23 | 66.80 | 77.84 | 72.32 |
| 38329 | 38521 | | | | | | | | | |
| WV- | WV- | 45.51 | 42.68 | 44.09 | 70.40 | 71.45 | 70.92 | 85.83 | 72.05 | 78.94 |
| 38332 | 38524 | | | | | | | | | |
| WV- | WV- | 31.29 | 36.03 | 33.66 | 43.23 | 40.39 | 41.81 | 43.18 | 36.72 | 39.95 |
| 38336 | 38528 | | | | | | | | | |
| WV- | WV- | 27.17 | 25.08 | 26.13 | 81.05 | 69.86 | 75.45 | 101.55 | 89.58 | 95.56 |
| 38337 | 38529 | | | | | | | | | |
| WV- | WV- | 47.97 | 46.39 | 47.18 | 76.87 | 81.63 | 79.25 | 84.28 | 108.88 | 96.58 |
| 38338 | 38530 | | | | | | | | | |
| WV- | WV- | 90.68 | N.D. | 90.68 | 104.01 | 105.16 | 104.58 | 112.43 | 107.28 | 109.86 |
| 38340 | 38532 | | | | | | | | | |
| WV- | WV- | N.D. | 103.19 | 103.19 | 84.72 | 112.96 | 98.84 | 112.11 | 115.31 | 113.71 |
| 38342 | 38534 | | | | | | | | | |
| WV- | WV- | 27.46 | N.D. | 27.46 | 77.01 | 73.20 | 75.11 | 86.42 | 93.21 | 89.81 |
| 38345 | 38537 | | | | | | | | | |
| WV- | WV- | 8.76 | 15.34 | 12.05 | 45.36 | 46.45 | 45.90 | 82.00 | 65.34 | 73.67 |
| 38346 | 38538 | | | | | | | | | |
| WV- | WV- | 35.43 | 42.08 | 38.76 | 68.19 | 81.38 | 74.79 | 88.04 | 77.47 | 82.76 |
| 38348 | 38540 | | | | | | | | | |
| WV- | WV- | 30.81 | N.D. | 30.81 | 58.74 | 49.58 | 54.16 | 79.56 | 55.53 | 67.54 |
| 38352 | 38544 | | | | | | | | | |
| WV- | WV- | 29.93 | 22.92 | 26.42 | 44.86 | 32.20 | 38.53 | 57.67 | 45.11 | 51.39 |
| 38354 | 38546 | | | | | | | | | |
| WV- | WV- | 26.48 | 26.03 | 26.25 | 55.47 | 50.19 | 52.83 | 90.79 | 77.65 | 84.22 |
| 38356 | 38548 | | | | | | | | | |
| WV- | WV- | 58.92 | 50.89 | 54.90 | 95.10 | 80.80 | 87.95 | 104.43 | 101.15 | 102.79 |
| 38357 | 38549 | | | | | | | | | |
| WV- | WV- | 21.84 | 34.10 | 27.97 | 70.54 | 63.67 | 67.11 | 103.64 | 67.65 | 85.64 |
| 38360 | 38552 | | | | | | | | | |
| WV- | WV- | 8.03 | 7.46 | 7.75 | 61.01 | 72.06 | 66.53 | 77.46 | 62.68 | 70.07 |
| 38361 | 38553 | | | | | | | | | |
| WV- | WV- | 27.95 | 28.41 | 28.18 | 65.60 | 62.28 | 63.94 | 61.62 | 69.10 | 65.36 |
| 38362 | 38554 | | | | | | | | | |
| WV- | WV- | 46.83 | 48.62 | 47.72 | 72.16 | 60.10 | 66.13 | 84.33 | 100.09 | 92.21 |
| 38365 | 38557 | | | | | | | | | |
| WV- | WV- | 31.75 | 19.19 | 25.47 | 67.87 | 70.38 | 69.13 | 83.12 | 113.84 | 98.48 |
| 38370 | 38562 | | | | | | | | | |

Table 4 shows % human HSD17B13 miRNA remaining (at 1, 0.3 and 0.1 nM by transfection) relative to human SFRS9 control. N=2. N.D.: Not determined.

TABLE 4

| | | 1 nM | | | 0.3 nM | | | 0.1 nM | | |
|---|---|---|---|---|---|---|---|---|---|---|
| | | % remaining | % remaining | | % remaining | % remaining | | % remaining | % remaining | |
| | | mRNA | mRNA | | mRNA | mRNA | | mRNA | mRNA | |
| | | (hHSD17B13/ | (hHSD17B13/ | | (hHSD17B13/ | (hHSD17B13/ | | (hHSD17B13/ | (hHSD17B13/ | |
| Guide | Passenger | hSFRS9)-1 | hSFRS9)-2 | Mean | hSFRS9)-1 | hSFRS9)-2 | Mean | hSFRS9)-1 | hSFRS9)-2 | Mean |
| WV- | WV- | 7.40 | 9.56 | 8.48 | 17.65 | 10.57 | 14.11 | 51.31 | 46.23 | 48.77 |
| 38240 | 38432 | | | | | | | | | |
| WV- | WV- | 11.91 | 11.41 | 11.66 | 22.21 | 17.20 | 19.70 | 40.41 | 42.70 | 41.56 |
| 38242 | 38434 | | | | | | | | | |
| WV- | WV- | 11.04 | 9.21 | 10.12 | 15.93 | 13.87 | 14.90 | 32.50 | 39.26 | 35.88 |
| 38246 | 38438 | | | | | | | | | |
| WV- | WV- | 11.56 | 11.18 | 11.37 | 32.30 | 13.23 | 22.76 | 21.72 | 23.06 | 22.39 |
| 38248 | 38440 | | | | | | | | | |
| WV- | WV- | 12.25 | 9.46 | 10.85 | 17.89 | 20.07 | 18.98 | 30.02 | 28.20 | 29.11 |
| 38249 | 38441 | | | | | | | | | |
| WV- | WV- | 9.42 | 3.55 | 6.48 | 14.98 | 14.11 | 14.55 | 35.93 | 46.25 | 41.09 |
| 38250 | 38442 | | | | | | | | | |
| WV- | WV- | 13.97 | 11.90 | 12.94 | 24.47 | 18.55 | 21.51 | 60.62 | 91.76 | 76.19 |
| 38257 | 38449 | | | | | | | | | |
| WV- | WV- | 9.60 | 10.63 | 10.11 | 19.77 | N.D. | 19.77 | 24.18 | N.D. | 24.18 |
| 38258 | 38450 | | | | | | | | | |
| WV- | WV- | 9.50 | 6.54 | 8.02 | 14.41 | 12.92 | 13.67 | 56.79 | 54.81 | 55.80 |
| 38269 | 38461 | | | | | | | | | |
| WV- | WV- | 6.43 | 10.41 | 8.42 | 24.57 | 24.51 | 24.54 | 51.76 | 44.63 | 48.19 |
| 38270 | 38462 | | | | | | | | | |
| WV- | WV- | 7.07 | 6.38 | 6.73 | 16.66 | 11.48 | 14.07 | 49.28 | 44.57 | 46.92 |
| 38279 | 38471 | | | | | | | | | |
| WV- | WV- | 13.24 | 9.00 | 11.12 | 60.58 | 28.95 | 44.76 | 67.89 | 55.83 | 61.86 |
| 38282 | 38474 | | | | | | | | | |
| WV- | WV- | 8.82 | 3.71 | 6.26 | 22.08 | 13.09 | 17.58 | 55.72 | 32.64 | 44.18 |
| 38284 | 38476 | | | | | | | | | |
| WV- | WV- | 15.95 | 15.18 | 15.56 | 76.08 | 68.53 | 72.31 | 47.97 | 163.51 | 105.74 |
| 38285 | 38477 | | | | | | | | | |
| WV- | WV- | 10.07 | 15.92 | 12.99 | 68.15 | 52.77 | 60.46 | 109.18 | 137.56 | 123.37 |
| 38290 | 38482 | | | | | | | | | |
| WV- | WV- | 12.05 | 6.99 | 9.52 | 29.98 | 23.22 | 26.60 | 45.15 | 99.50 | 72.33 |
| 38292 | 38484 | | | | | | | | | |
| WV- | WV- | 15.01 | 5.64 | 10.32 | 31.71 | 27.92 | 29.82 | 94.24 | 80.75 | 87.49 |
| 38293 | 38485 | | | | | | | | | |
| WV- | WV- | 7.47 | 7.30 | 7.38 | 25.68 | 19.54 | 22.61 | 70.27 | 51.56 | 60.92 |
| 38294 | 38486 | | | | | | | | | |
| WV- | WV- | 12.29 | 14.98 | 13.63 | 23.25 | 27.66 | 25.46 | 110.12 | 103.19 | 106.66 |
| 38296 | 38488 | | | | | | | | | |
| WV- | WV- | 23.27 | 17.51 | 20.39 | 53.00 | 102.24 | 77.62 | 102.86 | 137.35 | 120.11 |
| 38297 | 38489 | | | | | | | | | |
| WV- | WV- | 14.59 | 12.35 | 13.47 | 40.35 | 78.08 | 59.21 | 97.84 | 115.79 | 106.81 |
| 38298 | 38490 | | | | | | | | | |
| WV- | WV- | 8.34 | 12.71 | 10.53 | 29.70 | 57.42 | 43.56 | 93.52 | 113.02 | 103.27 |
| 38300 | 38492 | | | | | | | | | |
| WV- | WV- | 9.77 | 9.39 | 9.58 | 21.85 | 11.24 | 16.55 | 39.85 | 51.87 | 45.86 |
| 38301 | 38493 | | | | | | | | | |
| WV- | WV- | 4.30 | 5.19 | 4.75 | 49.48 | N.D. | 49.48 | 43.20 | 105.81 | 74.51 |
| 38305 | 38497 | | | | | | | | | |
| WV- | WV- | 21.47 | 21.48 | 21.47 | 65.66 | 67.23 | 66.45 | N.D. | 140.39 | 140.39 |
| 38306 | 38498 | | | | | | | | | |
| WV- | WV- | 12.43 | 12.36 | 12.39 | 54.88 | 37.06 | 45.97 | 89.48 | 135.68 | 112.58 |
| 38308 | 38500 | | | | | | | | | |
| WV- | WV- | 15.80 | 11.59 | 13.69 | 32.54 | 32.66 | 32.60 | 85.16 | 128.23 | 106.69 |
| 38312 | 38504 | | | | | | | | | |
| WV- | WV- | 7.57 | 4.82 | 6.19 | 85.38 | 47.21 | 66.30 | 85.54 | 75.45 | 80.49 |
| 38313 | 38505 | | | | | | | | | |
| WV- | WV- | 20.93 | 21.24 | 21.08 | 101.12 | 90.92 | 96.02 | 100.29 | 161.49 | 130.89 |
| 38316 | 38508 | | | | | | | | | |
| WV- | WV- | 14.69 | 11.18 | 12.94 | 34.94 | 43.65 | 39.29 | 72.34 | 131.03 | 101.68 |
| 38317 | 38509 | | | | | | | | | |
| WV- | WV- | 12.82 | 11.96 | 12.39 | 31.11 | 27.98 | 29.54 | 69.47 | 69.13 | 69.30 |
| 38318 | 38510 | | | | | | | | | |
| WV- | WV- | 8.20 | 7.90 | 8.05 | 24.05 | 40.60 | 32.32 | 100.63 | 118.32 | 109.48 |
| 38319 | 38511 | | | | | | | | | |
| WV- | WV- | 11.71 | 9.20 | 10.46 | 54.87 | 60.83 | 57.85 | 80.63 | 92.80 | 86.71 |
| 38322 | 38514 | | | | | | | | | |
| WV- | WV- | 5.68 | 2.30 | 3.99 | 16.53 | 15.90 | 16.21 | 45.40 | 45.93 | 45.66 |
| 38324 | 38516 | | | | | | | | | |
| WV- | WV- | 10.16 | 7.19 | 8.68 | 35.91 | 20.70 | 28.30 | 83.28 | 99.73 | 91.50 |
| 38325 | 38517 | | | | | | | | | |
| WV- | WV- | 3.99 | 4.54 | 4.27 | 37.08 | 27.76 | 32.42 | 45.30 | 45.80 | 45.55 |
| 38327 | 38519 | | | | | | | | | |
| WV- | WV- | 85.02 | 51.07 | 68.04 | 183.72 | 97.67 | 140.69 | 159.10 | 122.08 | 140.59 |
| 38329 | 38521 | | | | | | | | | |
| WV- | WV- | 4.24 | 11.18 | 7.71 | 77.90 | 70.61 | 74.26 | 172.36 | 98.78 | 135.57 |
| 38332 | 38524 | | | | | | | | | |
| WV- | WV- | 108.40 | 138.74 | 123.57 | 114.68 | 127.70 | 121.19 | 162.93 | 172.34 | 167.64 |
| 38336 | 38528 | | | | | | | | | |
| WV- | WV- | 14.85 | 17.14 | 15.99 | 41.59 | 55.47 | 48.53 | 114.71 | 160.45 | 137.58 |
| 38337 | 38529 | | | | | | | | | |
| WV- | WV- | 23.95 | 21.44 | 22.70 | 60.94 | 50.41 | 55.68 | 86.06 | 108.97 | 97.51 |
| 38338 | 38530 | | | | | | | | | |
| WV- | WV- | 9.86 | 10.15 | 10.00 | 35.34 | 40.55 | 37.94 | 91.09 | 109.60 | 100.34 |
| 38339 | 38531 | | | | | | | | | |
| WV- | WV- | 74.50 | 34.95 | 54.73 | 94.21 | 79.27 | 86.74 | 158.39 | 124.03 | 141.21 |
| 38340 | 38532 | | | | | | | | | |
| WV- | WV- | 31.41 | 14.99 | 23.20 | 61.15 | 92.64 | 76.90 | 136.20 | 170.37 | 153.28 |
| 38342 | 38534 | | | | | | | | | |
| WV- | WV- | 9.36 | 16.83 | 13.09 | 68.19 | 89.43 | 78.81 | 117.72 | 145.78 | 131.75 |
| 38345 | 38537 | | | | | | | | | |
| WV- | WV- | 10.97 | 7.80 | 9.38 | 54.56 | 36.73 | 45.65 | 102.08 | 103.44 | 102.76 |
| 38346 | 38538 | | | | | | | | | |
| WV- | WV- | 7.61 | 6.33 | 6.97 | 24.03 | 22.11 | 23.07 | 64.74 | 25.62 | 45.18 |
| 38347 | 38539 | | | | | | | | | |
| WV- | WV- | 31.95 | 28.13 | 30.04 | 89.71 | 108.02 | 98.86 | 145.47 | 163.83 | 154.65 |
| 38348 | 38540 | | | | | | | | | |
| WV- | WV- | 42.40 | 44.35 | 43.38 | 79.22 | 118.92 | 99.07 | 122.70 | 177.81 | 150.25 |
| 38351 | 38543 | | | | | | | | | |
| WV- | WV- | 6.87 | 7.13 | 7.00 | 29.95 | 25.84 | 27.89 | 103.20 | 49.77 | 76.48 |
| 38352 | 38544 | | | | | | | | | |
| WV- | WV- | 20.17 | 15.46 | 17.82 | 40.31 | 40.48 | 40.40 | 92.69 | 129.94 | 111.32 |
| 38354 | 38546 | | | | | | | | | |
| WV- | WV- | 5.99 | 5.73 | 5.86 | 58.78 | 51.97 | 55.38 | 50.97 | 66.29 | 58.63 |
| 38355 | 38547 | | | | | | | | | |
| WV- | WV- | 7.89 | 7.04 | 7.46 | 26.92 | 32.18 | 29.55 | 93.47 | 91.82 | 92.65 |
| 38356 | 38548 | | | | | | | | | |
| WV- | WV- | 21.07 | 17.50 | 19.29 | 84.83 | 51.41 | 68.12 | 115.00 | 109.47 | 112.24 |
| 38357 | 38549 | | | | | | | | | |
| WV- | WV- | 5.65 | 10.25 | 7.95 | 18.08 | 35.60 | 26.84 | 88.37 | 103.44 | 95.90 |
| 38359 | 38551 | | | | | | | | | |
| WV- | WV- | 12.26 | 8.71 | 10.48 | 51.58 | 45.82 | 48.70 | 147.72 | 111.61 | 129.67 |
| 38360 | 38552 | | | | | | | | | |
| WV- | WV- | 9.62 | 4.67 | 7.15 | 27.86 | 25.42 | 26.64 | 70.30 | 88.91 | 79.60 |
| 38361 | 38553 | | | | | | | | | |
| WV- | WV- | 11.84 | 8.18 | 10.01 | 77.67 | 65.78 | 71.72 | 89.07 | 127.97 | 108.52 |
| 38362 | 38554 | | | | | | | | | |
| WV- | WV- | 17.38 | 16.05 | 16.71 | 38.50 | 36.40 | 37.45 | 107.95 | 137.63 | 122.79 |
| 38365 | 38557 | | | | | | | | | |
| WV- | WV- | 12.74 | 10.34 | 11.54 | 48.48 | 39.88 | 44.18 | 64.89 | 74.29 | 69.59 |
| 38370 | 38562 | | | | | | | | | |
| WV- | WV- | 20.23 | N.D. | 20.23 | 50.88 | 66.73 | 58.80 | 110.78 | 103.38 | 107.08 |
| 38404 | 38596 | | | | | | | | | |
| WV- | WV- | 32.26 | 23.04 | 27.65 | 43.15 | 28.68 | 35.91 | 80.65 | 78.94 | 79.79 |
| 38409 | 38601 | | | | | | | | | |
| WV- | WV- | 33.91 | 24.99 | 29.45 | 27.57 | 13.87 | 20.72 | 49.57 | 65.32 | 57.44 |
| 38411 | 38603 | | | | | | | | | |
| WV- | WV- | 35.92 | 26.53 | 31.23 | 56.84 | 56.53 | 56.69 | 106.35 | 109.95 | 108.15 |
| 38415 | 38607 | | | | | | | | | |

Table 5 shows % human HSD17B13 miRNA remaining (at 1, 0.3 and 0.1 nM by transfection) relative to human SFRS9 control. N=2. N.D.: Not determined.

TABLE 5

| | | 1 nM | | | 0.3 nM | | | 0.1 nM | | |
|---|---|---|---|---|---|---|---|---|---|---|
| | | % remaining | % remaining | | % remaining | % remaining | | % remaining | % remaining | |
| | | mRNA | mRNA | | mRNA | mRNA | | mRNA | mRNA | |
| | | (hHSD17B13/ | (hHSD17B13/ | | (hHSD17B13/ | (hHSD17B13/ | | (hHSD17B13/ | (hHSD17B13/ | |
| Guide | Passenger | hSFRS9)-1 | hSFRS9)-2 | Mean | hSFRS9)-1 | hSFRS9)-2 | Mean | hSFRS9)-1 | hSFRS9)-2 | Mean |
| WV- | WV- | 33.41 | 23.59 | 28.50 | 52.17 | 64.79 | 58.48 | 66.54 | 77.57 | 72.05 |
| 42129 | 42219 | | | | | | | | | |
| WV- | WV- | 31.51 | 14.73 | 23.12 | 56.10 | 37.78 | 46.94 | 74.05 | 73.97 | 74.01 |
| 42130 | 42220 | | | | | | | | | |
| WV- | WV- | 33.57 | 54.63 | 44.10 | 106.76 | 179.91 | 143.33 | 100.32 | 141.91 | 121.12 |
| 42131 | 42221 | | | | | | | | | |
| WV- | WV- | 27.30 | 17.91 | 22.60 | 56.61 | 54.86 | 55.73 | 47.56 | 70.14 | 58.85 |
| 42132 | 42222 | | | | | | | | | |
| WV- | WV- | 26.42 | 30.03 | 28.23 | 70.09 | 54.93 | 62.51 | 78.14 | 60.53 | 69.34 |
| 42133 | 42223 | | | | | | | | | |
| WV- | WV- | 41.24 | 28.10 | 34.67 | 60.65 | 69.58 | 65.11 | 70.83 | 87.26 | 79.05 |
| 42134 | 42224 | | | | | | | | | |
| WV- | WV- | 38.66 | 23.33 | 30.99 | 76.74 | 59.32 | 68.03 | 75.15 | 86.15 | 80.65 |
| 42135 | 42225 | | | | | | | | | |
| WV- | WV- | 32.70 | 24.12 | 28.41 | 59.27 | 73.91 | 66.59 | 86.08 | 75.87 | 80.97 |
| 42136 | 42226 | | | | | | | | | |
| WV- | WV- | 28.88 | 12.48 | 20.68 | 31.58 | 43.53 | 37.55 | 52.71 | 37.81 | 45.26 |
| 42137 | 42227 | | | | | | | | | |
| WV- | WV- | 22.58 | 21.29 | 21.93 | 44.36 | 43.09 | 43.73 | 46.89 | 63.98 | 55.43 |
| 42138 | 42228 | | | | | | | | | |
| WV- | WV- | 44.52 | 52.34 | 48.43 | 101.36 | 96.93 | 99.14 | 90.74 | 103.19 | 96.96 |
| 42139 | 42229 | | | | | | | | | |
| WV- | WV- | 30.50 | 25.22 | 27.86 | 62.75 | 45.58 | 54.16 | 73.14 | 75.84 | 74.49 |
| 42140 | 42230 | | | | | | | | | |
| WV- | WV- | 25.47 | 20.59 | 23.03 | 40.48 | 60.30 | 50.39 | 57.34 | 52.90 | 55.12 |
| 42141 | 42231 | | | | | | | | | |
| WV- | WV- | 23.83 | 26.14 | 24.99 | 45.51 | 50.17 | 47.84 | 69.28 | 72.15 | 70.72 |
| 42142 | 42232 | | | | | | | | | |
| WV- | WV- | 16.76 | 23.46 | 20.11 | 40.79 | 37.74 | 39.27 | 32.10 | 28.55 | 30.33 |
| 42143 | 42233 | | | | | | | | | |
| WV- | WV- | 25.64 | 19.12 | 22.38 | 38.01 | 37.57 | 37.79 | 37.37 | 35.07 | 36.22 |
| 42144 | 42234 | | | | | | | | | |
| WV- | WV- | 35.23 | 19.57 | 27.40 | 44.58 | 38.35 | 41.46 | 28.51 | 34.89 | 31.70 |
| 42145 | 42235 | | | | | | | | | |
| WV- | WV- | 27.75 | 18.98 | 23.37 | 29.12 | 29.21 | 29.16 | 29.39 | 41.98 | 35.69 |
| 42146 | 42236 | | | | | | | | | |
| WV- | WV- | 33.54 | 22.46 | 28.00 | 46.05 | 35.85 | 40.95 | 57.80 | 66.47 | 62.13 |
| 42147 | 42237 | | | | | | | | | |
| WV- | WV- | 25.21 | 22.28 | 23.75 | 48.93 | 29.97 | 39.45 | 44.03 | 37.53 | 40.78 |
| 42148 | 42238 | | | | | | | | | |
| WV- | WV- | 45.67 | 41.79 | 43.73 | 58.41 | 65.87 | 62.14 | 59.87 | 63.86 | 61.87 |
| 42149 | 42239 | | | | | | | | | |
| WV- | WV- | 61.28 | 40.91 | 51.10 | 80.92 | 65.54 | 73.23 | 70.75 | 75.99 | 73.37 |
| 42150 | 42240 | | | | | | | | | |
| WV- | WV- | 45.54 | 35.91 | 40.72 | 59.86 | 76.33 | 68.09 | 64.54 | 49.22 | 56.88 |
| 42151 | 42241 | | | | | | | | | |
| WV- | WV- | 52.21 | 104.97 | 78.59 | 77.21 | 64.12 | 70.66 | 65.83 | 76.66 | 71.25 |
| 42152 | 42242 | | | | | | | | | |
| WV- | WV- | 53.77 | 20.02 | 36.89 | 66.30 | 55.01 | 60.66 | 53.93 | 58.93 | 56.43 |
| 42153 | 42243 | | | | | | | | | |
| WV- | WV- | 51.46 | 41.43 | 46.45 | 65.60 | 69.19 | 67.39 | 75.45 | 73.39 | 74.42 |
| 42154 | 42244 | | | | | | | | | |
| WV- | WV- | 52.58 | 31.10 | 41.84 | 76.42 | 68.58 | 72.50 | 66.43 | 85.48 | 75.95 |
| 42155 | 42245 | | | | | | | | | |
| WV- | WV- | 52.87 | 43.66 | 48.26 | 67.81 | 69.47 | 68.64 | 79.86 | 80.66 | 80.26 |
| 42156 | 42246 | | | | | | | | | |
| WV- | WV- | 59.58 | 28.56 | 44.07 | 70.38 | 57.86 | 64.12 | 52.67 | 52.61 | 52.64 |
| 42157 | 42247 | | | | | | | | | |
| WV- | WV- | 50.77 | 29.03 | 39.90 | 56.09 | 57.02 | 56.55 | 62.85 | 66.53 | 64.69 |
| 42158 | 42248 | | | | | | | | | |
| WV- | WV- | 48.14 | 26.70 | 37.42 | 73.62 | 64.32 | 68.97 | 59.52 | 64.29 | 61.90 |
| 42159 | 42249 | | | | | | | | | |
| WV- | WV- | 49.76 | 47.70 | 48.73 | 57.23 | 59.48 | 58.36 | 65.75 | 66.81 | 66.28 |
| 42160 | 42250 | | | | | | | | | |
| WV- | WV- | 49.04 | 43.06 | 46.05 | 70.40 | 55.65 | 63.03 | 58.82 | 69.81 | 64.31 |
| 42161 | 42251 | | | | | | | | | |
| WV- | WV- | 52.58 | 31.44 | 42.01 | 64.50 | 55.02 | 59.76 | 58.06 | 81.86 | 69.96 |
| 42162 | 42252 | | | | | | | | | |
| WV- | WV- | 37.95 | 33.22 | 35.59 | 58.53 | 71.43 | 64.98 | 49.50 | 52.81 | 51.15 |
| 42163 | 42253 | | | | | | | | | |
| WV- | WV- | 57.27 | 26.61 | 41.94 | 48.36 | 55.74 | 52.05 | 60.29 | 73.70 | 66.99 |
| 42164 | 42254 | | | | | | | | | |
| WV- | WV- | 48.50 | 42.17 | 45.33 | 68.28 | 70.18 | 69.23 | 58.55 | 76.30 | 67.43 |
| 42165 | 42255 | | | | | | | | | |
| WV- | WV- | 52.40 | 41.00 | 46.70 | 87.62 | 67.12 | 77.37 | 75.79 | 49.89 | 62.84 |
| 42166 | 42256 | | | | | | | | | |
| WV- | WV- | 67.61 | 46.91 | 57.26 | 70.13 | 66.61 | 68.37 | 58.56 | 59.22 | 58.89 |
| 42167 | 42257 | | | | | | | | | |
| WV- | WV- | 52.20 | 28.59 | 40.39 | 55.08 | 61.28 | 58.18 | 80.46 | 81.27 | 80.86 |
| 42168 | 42258 | | | | | | | | | |
| WV- | WV- | 44.01 | 39.06 | 41.54 | 60.07 | 54.72 | 57.40 | 57.40 | 57.26 | 57.33 |
| 42169 | 42259 | | | | | | | | | |
| WV- | WV- | 61.05 | 45.98 | 53.52 | 72.79 | 67.03 | 69.91 | 53.81 | 75.53 | 64.67 |
| 42170 | 42260 | | | | | | | | | |
| WV- | WV- | 59.20 | 49.91 | 54.55 | 82.89 | 80.25 | 81.57 | 53.44 | 68.47 | 60.95 |
| 42171 | 42261 | | | | | | | | | |
| WV- | WV- | 56.18 | 51.53 | 53.85 | 92.25 | 72.05 | 82.15 | 71.82 | 99.78 | 85.80 |
| 42172 | 42262 | | | | | | | | | |
| WV- | WV- | 45.65 | 47.95 | 46.80 | 71.41 | 79.69 | 75.55 | 53.34 | 68.14 | 60.74 |
| 42173 | 42263 | | | | | | | | | |
| WV- | WV- | 51.60 | 36.29 | 43.94 | 69.42 | 65.90 | 67.66 | 57.36 | 65.94 | 61.65 |
| 42174 | 42264 | | | | | | | | | |
| WV- | WV- | 48.24 | 27.30 | 37.77 | 60.34 | 62.74 | 61.54 | 62.51 | 52.38 | 57.44 |
| 42175 | 42265 | | | | | | | | | |
| WV- | WV- | 43.91 | 41.45 | 42.68 | 62.89 | 72.80 | 67.85 | 66.11 | 74.87 | 70.49 |
| 42176 | 42266 | | | | | | | | | |
| WV- | WV- | 40.17 | 34.33 | 37.25 | 71.39 | 62.15 | 66.77 | 49.82 | 56.20 | 53.01 |
| 42177 | 42267 | | | | | | | | | |
| WV- | WV- | 66.35 | 37.53 | 51.94 | 74.79 | 71.69 | 73.24 | 66.62 | 58.59 | 62.60 |
| 42178 | 42268 | | | | | | | | | |
| WV- | WV- | 43.48 | 35.79 | 39.64 | 61.14 | 54.84 | 57.99 | 63.43 | 63.37 | 63.40 |
| 42179 | 42269 | | | | | | | | | |
| WV- | WV- | 61.53 | 40.84 | 51.19 | 75.88 | 65.13 | 70.51 | 78.48 | 99.95 | 89.21 |
| 42180 | 42270 | | | | | | | | | |
| WV- | WV- | 54.82 | 24.96 | 39.89 | 84.30 | 87.09 | 85.69 | 105.62 | 114.65 | 110.14 |
| 42181 | 42271 | | | | | | | | | |
| WV- | WV- | 58.50 | 34.97 | 46.74 | 49.17 | 59.56 | 54.36 | 67.07 | 67.47 | 67.27 |
| 42182 | 42272 | | | | | | | | | |
| WV- | WV- | 50.15 | 19.65 | 34.90 | 57.00 | 60.44 | 58.72 | 56.37 | 60.48 | 58.42 |
| 42183 | 42273 | | | | | | | | | |
| WV- | WV- | 53.79 | 42.47 | 48.13 | 64.59 | 66.32 | 65.45 | 57.23 | 59.65 | 58.44 |
| 42184 | 42274 | | | | | | | | | |
| WV- | WV- | 48.67 | 37.16 | 42.91 | 75.33 | 45.13 | 60.23 | 45.48 | 65.42 | 55.45 |
| 42185 | 42275 | | | | | | | | | |
| WV- | WV- | 76.70 | 50.69 | 63.70 | 93.39 | 77.79 | 85.59 | 87.22 | 106.07 | 96.64 |
| 42186 | 42276 | | | | | | | | | |
| WV- | WV- | 65.32 | 47.32 | 56.32 | 62.65 | 75.07 | 68.86 | 81.79 | 86.23 | 84.01 |
| 42187 | 42277 | | | | | | | | | |
| WV- | WV- | 54.10 | 38.38 | 46.24 | 68.39 | 65.03 | 66.71 | 69.17 | 81.00 | 75.09 |
| 42188 | 42278 | | | | | | | | | |
| WV- | WV- | 61.25 | 27.80 | 44.52 | 82.77 | 79.38 | 81.08 | 69.27 | 97.33 | 83.30 |
| 42189 | 42279 | | | | | | | | | |
| WV- | WV- | 55.42 | 28.54 | 41.98 | 80.61 | 76.59 | 78.60 | 54.27 | 66.93 | 60.60 |
| 42190 | 42280 | | | | | | | | | |
| WV- | WV- | 51.43 | 38.58 | 45.00 | 58.06 | 55.70 | 56.88 | 65.34 | 61.23 | 63.29 |
| 42191 | 42281 | | | | | | | | | |
| WV- | WV- | 51.79 | 32.08 | 41.93 | 64.43 | 57.80 | 61.11 | 66.94 | 73.91 | 70.43 |
| 42192 | 42282 | | | | | | | | | |
| WV- | WV- | 42.50 | 33.38 | 37.94 | 74.32 | 75.22 | 74.77 | 65.70 | 63.35 | 64.52 |
| 42193 | 42283 | | | | | | | | | |
| WV- | WV- | 57.64 | 41.90 | 49.77 | 62.57 | 62.98 | 62.77 | 62.07 | 60.72 | 61.39 |
| 42194 | 42284 | | | | | | | | | |
| WV- | WV- | 49.79 | 32.25 | 41.02 | 65.16 | 83.13 | 74.15 | 56.73 | 61.66 | 59.20 |
| 42195 | 42285 | | | | | | | | | |
| WV- | WV- | 59.93 | 40.69 | 50.31 | 59.61 | 57.21 | 58.41 | 54.53 | 72.47 | 63.50 |
| 42196 | 42286 | | | | | | | | | |
| WV- | WV- | 58.06 | 39.03 | 48.55 | 64.87 | 56.77 | 60.82 | 91.87 | 81.05 | 86.46 |
| 42197 | 42287 | | | | | | | | | |
| WV- | WV- | 63.16 | 50.05 | 56.61 | 78.06 | 78.80 | 78.43 | 66.88 | 64.96 | 65.92 |
| 42198 | 42288 | | | | | | | | | |
| WV- | WV- | 46.16 | 31.59 | 38.87 | 77.46 | 55.35 | 66.41 | 39.74 | 58.82 | 49.28 |
| 42199 | 42289 | | | | | | | | | |
| WV- | WV- | 43.30 | 27.99 | 35.65 | 61.50 | 48.84 | 55.17 | 79.36 | 65.62 | 72.49 |
| 42200 | 42290 | | | | | | | | | |
| WV- | WV- | 49.83 | 41.79 | 45.81 | 65.57 | 60.32 | 62.94 | 59.49 | 54.36 | 56.93 |
| 42201 | 42291 | | | | | | | | | |
| WV- | WV- | 53.97 | 48.39 | 51.18 | 60.85 | 42.93 | 51.89 | 63.96 | 73.19 | 68.58 |
| 42202 | 42292 | | | | | | | | | |
| WV- | WV- | 54.49 | 46.70 | 50.59 | 68.01 | 67.34 | 67.68 | 61.22 | 57.62 | 59.42 |
| 42203 | 42293 | | | | | | | | | |
| WV- | WV- | 48.47 | 47.65 | 48.06 | 62.30 | 65.85 | 64.08 | 59.92 | 61.17 | 60.55 |
| 42204 | 42294 | | | | | | | | | |
| WV- | WV- | 54.35 | 42.48 | 48.41 | 67.90 | 54.66 | 61.28 | 59.66 | 60.79 | 60.22 |
| 42205 | 42295 | | | | | | | | | |
| WV- | WV- | 59.66 | 49.79 | 54.72 | 70.88 | 69.94 | 70.41 | 70.98 | 64.93 | 67.95 |
| 42206 | 42296 | | | | | | | | | |
| WV- | WV- | 42.45 | 27.93 | 35.19 | 92.31 | 48.19 | 70.25 | 57.52 | 54.11 | 55.82 |
| 42207 | 42297 | | | | | | | | | |
| WV- | WV- | 56.80 | 40.44 | 48.62 | 64.22 | 64.62 | 64.42 | 57.42 | 82.20 | 69.81 |
| 42208 | 42298 | | | | | | | | | |
| WV- | WV- | 70.38 | 42.01 | 56.20 | 75.39 | 79.92 | 77.65 | 80.46 | 97.28 | 88.87 |
| 42209 | 42299 | | | | | | | | | |
| WV- | WV- | 57.41 | 36.84 | 47.13 | 79.11 | 86.13 | 82.62 | 62.32 | 56.12 | 59.22 |
| 42210 | 42300 | | | | | | | | | |
| WV- | WV- | 69.63 | 57.75 | 63.69 | 115.63 | 117.76 | 116.70 | 111.23 | 112.74 | 111.98 |
| 42211 | 42301 | | | | | | | | | |
| WV- | WV- | 55.90 | 36.39 | 46.15 | 77.88 | 52.75 | 65.31 | 52.58 | 66.17 | 59.37 |
| 42212 | 42302 | | | | | | | | | |
| WV- | WV- | 70.01 | 33.10 | 51.56 | 72.13 | 67.94 | 70.04 | 98.59 | 106.17 | 102.38 |
| 42213 | 42303 | | | | | | | | | |
| WV- | WV- | 32.81 | 46.49 | 39.65 | 75.37 | 60.16 | 67.76 | 62.22 | 56.80 | 59.51 |
| 42214 | 42304 | | | | | | | | | |
| WV- | WV- | 53.92 | 42.67 | 48.29 | 65.13 | 57.07 | 61.10 | 56.95 | 58.98 | 57.96 |
| 42215 | 42305 | | | | | | | | | |
| WV- | WV- | 57.20 | 32.27 | 44.73 | 87.57 | 63.49 | 75.53 | 51.78 | 54.90 | 53.34 |
| 42216 | 42306 | | | | | | | | | |
| WV- | WV- | 45.86 | 43.05 | 44.46 | 66.75 | 55.81 | 61.28 | 53.70 | 73.52 | 63.61 |
| 42217 | 42307 | | | | | | | | | |
| WV- | WV- | 62.78 | 65.79 | 64.29 | 108.87 | 87.57 | 98.22 | 87.49 | 100.52 | 94.01 |
| 42218 | 42308 | | | | | | | | | |

Table 6 shows % NHP HSD17B13 nmRNA remaining (at 10 and 1 nM by transfection) relative to NHP SFRS9 control. N=2. N.D.: Not determined.

TABLE 6

| | | 10 nM | | | 1 nM | | |
|---|---|---|---|---|---|---|---|
| | | % remaining | % remaining | | % remaining | % remaining | |
| | | mRNA | mRNA | | mRNA | mRNA | |
| | | (MfHSD17B13/ | (MfHSD17B13/ | | (MfHSD17B13/ | (MfHSD17B13/ | |
| Guide | Passenger | MfSFRS9)-1 | MfSFRS9)-2 | Mean | MfSFRS9)-1 | MfSFRS9)-2 | Mean |
| WV-42145 | WV-42235 | 5.18 | 4.79 | 4.98 | 11.47 | 8.28 | 9.88 |
| WV-42146 | WV-42236 | 4.16 | N.D. | 4.16 | 11.00 | 10.77 | 10.88 |
| WV-42147 | WV-42237 | 7.24 | 33.16 | 20.20 | 12.75 | 20.17 | 16.46 |
| WV-42148 | WV-42238 | N.D. | 10.28 | 10.28 | 12.19 | 9.05 | 10.62 |
| WV-42149 | WV-42239 | 12.57 | 15.90 | 14.24 | 73.37 | 16.81 | 45.09 |
| WV-42150 | WV-42240 | 39.47 | 24.51 | 31.99 | 45.29 | 35.26 | 40.28 |
| WV-42151 | WV-42241 | 6.32 | 17.58 | 11.95 | 20.23 | 40.42 | 30.32 |
| WV-42152 | WV-42242 | 11.85 | 13.26 | 12.56 | 22.65 | 70.59 | 46.62 |
| WV-42153 | WV-42243 | N.D. | 9.59 | 9.59 | 8.98 | 13.82 | 11.40 |
| WV-42154 | WV-42244 | 7.51 | 15.52 | 11.51 | 16.52 | 8.51 | 12.51 |
| WV-42155 | WV-42245 | 16.05 | 13.72 | 14.89 | 92.91 | N.D. | 92.91 |
| WV-42156 | WV-42246 | 4.58 | 23.20 | 13.89 | 20.75 | 80.74 | 50.75 |
| WV-42157 | WV-42247 | N.D. | 14.25 | 14.25 | 28.72 | 7.46 | 18.09 |
| WV-42158 | WV-42248 | 11.37 | 17.03 | 14.20 | 30.25 | 13.52 | 21.88 |
| WV-42159 | WV-42249 | 16.07 | 27.77 | 21.92 | 18.77 | 32.62 | 25.69 |
| WV-42160 | WV-42250 | 4.94 | 23.46 | 14.20 | 20.03 | 33.23 | 26.63 |
| WV-42161 | WV-42251 | 9.54 | 6.40 | 7.97 | 24.23 | 8.78 | 16.50 |
| WV-42162 | WV-42252 | 14.73 | 40.81 | 27.77 | 38.95 | 46.71 | 42.83 |
| WV-42163 | WV-42253 | 6.90 | 12.10 | 9.50 | 24.67 | 28.49 | 26.58 |
| WV-42164 | WV-42254 | 8.11 | 14.26 | 11.18 | 23.71 | 13.90 | 18.81 |
| WV-42165 | WV-42255 | 20.77 | 24.20 | 22.48 | 29.09 | 16.25 | 22.67 |
| WV-42166 | WV-42256 | 17.77 | 27.11 | 22.44 | 29.93 | 45.16 | 37.55 |
| WV-42167 | WV-42257 | 19.02 | 36.58 | 27.80 | 62.11 | 75.34 | 68.73 |
| WV-42168 | WV-42258 | 7.85 | 18.84 | 13.34 | 17.12 | 35.25 | 26.18 |
| WV-42169 | WV-42259 | 16.94 | 40.67 | 28.80 | 52.76 | 15.40 | 34.08 |
| WV-42170 | WV-42260 | 26.92 | 17.99 | 22.45 | 55.68 | 34.83 | 45.25 |
| WV-42171 | WV-42261 | 27.30 | 31.09 | 29.20 | 60.64 | 27.54 | 44.09 |
| WV-42172 | WV-42262 | 17.61 | 37.26 | 27.44 | 44.42 | 22.19 | 33.31 |
| WV-42173 | WV-42263 | 15.60 | 18.13 | 16.86 | 32.59 | 26.09 | 29.34 |
| WV-42174 | WV-42264 | 41.13 | 17.01 | 29.07 | 65.85 | 25.55 | 45.70 |
| WV-42175 | WV-42265 | 8.13 | 6.88 | 7.50 | 48.28 | 130.53 | 89.40 |
| WV-42176 | WV-42266 | 24.43 | 7.49 | 15.96 | 47.50 | 24.27 | 35.88 |
| WV-42177 | WV-42267 | 6.25 | 13.54 | 9.90 | 18.72 | 29.71 | 24.22 |
| WV-42178 | WV-42268 | 8.61 | 12.23 | 10.42 | 20.59 | 19.03 | 19.81 |
| WV-42179 | WV-42269 | 4.66 | 7.31 | 5.99 | 23.19 | 38.94 | 31.07 |
| WV-42180 | WV-42270 | 11.60 | 24.47 | 18.04 | 72.02 | N.D. | 72.02 |
| WV-42181 | WV-42271 | 18.30 | 24.12 | 21.21 | 83.65 | 24.31 | 53.98 |
| WV-42182 | WV-42272 | 6.49 | 24.18 | 15.33 | 81.16 | 11.92 | 46.54 |
| WV-42183 | WV-42273 | 8.87 | 10.15 | 9.51 | 30.34 | 21.67 | 26.00 |
| WV-42184 | WV-42274 | 12.73 | N.D. | 12.73 | 28.01 | 24.23 | 26.12 |
| WV-42185 | WV-42275 | 12.94 | 26.11 | 19.53 | 36.73 | 12.13 | 24.43 |
| WV-42186 | WV-42276 | 22.23 | 34.28 | 28.25 | 41.48 | 32.94 | 37.21 |
| WV-42187 | WV-42277 | 23.39 | 11.04 | 17.22 | 24.37 | 22.26 | 23.32 |
| WV-42188 | WV-42278 | 27.45 | 13.54 | 20.49 | 26.94 | 42.11 | 34.52 |
| WV-42189 | WV-42279 | 30.43 | 23.96 | 27.19 | 55.09 | 10.98 | 33.03 |
| WV-42190 | WV-42280 | 70.31 | 19.61 | 44.96 | 88.41 | 14.95 | 51.68 |
| WV-42191 | WV-42281 | 7.71 | N.D. | 7.71 | 19.10 | 17.88 | 18.49 |
| WV-42192 | WV-42282 | 7.57 | N.D. | 7.57 | 13.06 | 13.11 | 13.08 |
| WV-42193 | WV-42283 | N.D. | 10.46 | 10.46 | 25.36 | 27.12 | 26.24 |
| WV-42194 | WV-42284 | 18.31 | 21.65 | 19.98 | 14.98 | 14.15 | 14.57 |
| WV-42195 | WV-42285 | 18.49 | 18.59 | 18.54 | 11.57 | 19.54 | 15.56 |
| WV-42196 | WV-42286 | 13.19 | 21.69 | 17.44 | 27.48 | 36.42 | 31.95 |
| WV-42197 | WV-42287 | 8.96 | 9.36 | 9.16 | 19.42 | 14.38 | 16.90 |
| WV-42198 | WV-42288 | 13.37 | 30.72 | 22.05 | 44.61 | 25.90 | 35.26 |
| WV-42199 | WV-42289 | 15.25 | 37.61 | 26.43 | 29.02 | 18.78 | 23.90 |
| WV-42200 | WV-42290 | 7.75 | 16.28 | 12.01 | 17.48 | 14.33 | 15.90 |
| WV-42201 | WV-42291 | 13.07 | 10.76 | 11.92 | 10.12 | 10.07 | 10.09 |
| WV-42202 | WV-42292 | 17.04 | 28.88 | 22.96 | 21.13 | 26.38 | 23.76 |
| WV-42203 | WV-42293 | 15.27 | 33.51 | 24.39 | 20.60 | 18.69 | 19.65 |
| WV-42204 | WV-42294 | 26.34 | 26.54 | 26.44 | 19.49 | 25.05 | 22.27 |
| WV-42205 | WV-42295 | 15.36 | 34.90 | 25.13 | 41.71 | 13.70 | 27.71 |
| WV-42206 | WV-42296 | 12.07 | 45.29 | 28.68 | 43.05 | 16.65 | 29.85 |
| WV-42207 | WV-42297 | 22.45 | 27.73 | 25.09 | 42.96 | 13.78 | 28.37 |
| WV-42208 | WV-42298 | 9.22 | 4.63 | 6.92 | 20.04 | 37.66 | 28.85 |
| WV-42209 | WV-42299 | 46.31 | 48.58 | 47.44 | 28.08 | 24.69 | 26.39 |
| WV-42210 | WV-42300 | 10.36 | 27.63 | 18.99 | 37.21 | 16.71 | 26.96 |
| WV-42211 | WV-42301 | 43.68 | 47.87 | 45.78 | 39.72 | 47.94 | 43.83 |
| WV-42212 | WV-42302 | 65.88 | 28.28 | 47.08 | 26.37 | 30.73 | 28.55 |
| WV-42213 | WV-42303 | 24.50 | 19.83 | 22.16 | 24.76 | 35.64 | 30.20 |
| WV-42214 | WV-42304 | 46.32 | 28.99 | 37.65 | 35.05 | 23.93 | 29.49 |
| WV-42215 | WV-42305 | N.D. | N.D. | N.D. | 36.49 | 22.34 | 29.41 |
| WV-42216 | WV-42306 | 30.32 | N.D. | 30.32 | 30.86 | 68.94 | 49.90 |
| WV-42217 | WV-42307 | 11.23 | 29.34 | 20.28 | 11.32 | 16.60 | 13.96 |
| WV-42218 | WV-42308 | 91.69 | 87.99 | 89.84 | 100.42 | 68.20 | 84.31 |

Table 7 shows % human HSD17B13 miRNA remaining (at 30 and 10 nM by gymnotic delivery) relative to human SFRS9 control. N=2. N.D.: Not determined.

TABLE 7

| | | 30 nM | | | 10 nM | | |
|---|---|---|---|---|---|---|---|
| | | % remaining | % remaining | | % remaining | % remaining | |
| | | mRNA | mRNA | | mRNA | mRNA | |
| | | (hHSD17B13/ | (hHSD17B13/ | | (hHSD17B13/ | (hHSD17B13/ | |
| Guide | Passenger | hSFRS9)-1 | hSFRS9)-2 | Mean | hSFRS9)-1 | hSFRS9)-2 | Mean |
| WV-42354 | WV-42531 | 46.21 | 44.83 | 45.52 | 76.13 | 73.46 | 74.79 |
| WV-42355 | WV-42532 | 57.13 | 65.92 | 61.52 | 68.63 | 73.22 | 70.92 |
| WV-42356 | WV-42533 | 28.47 | 33.71 | 31.09 | 55.11 | 52.57 | 53.84 |
| WV-42357 | WV-42534 | 34.23 | 51.11 | 42.67 | 71.40 | 54.62 | 63.01 |
| WV-42358 | WV-42535 | 44.99 | 53.10 | 49.04 | 81.73 | 64.41 | 73.07 |
| WV-42359 | WV-42536 | 50.03 | 55.59 | 52.81 | 77.51 | 77.92 | 77.72 |
| WV-42360 | WV-42537 | 54.55 | 75.02 | 64.79 | 98.69 | 75.46 | 87.08 |
| WV-42361 | WV-42538 | 37.79 | N.D. | 37.79 | 65.95 | 61.24 | 63.60 |
| WV-42362 | WV-42539 | 48.24 | 49.66 | 48.95 | 64.97 | 54.78 | 59.88 |
| WV-42363 | WV-42540 | 48.91 | 41.53 | 45.22 | 61.48 | 55.93 | 58.71 |
| WV-42364 | WV-42541 | 45.24 | 49.78 | 47.51 | 66.77 | 71.74 | 69.26 |
| WV-42365 | WV-42542 | 71.19 | 96.01 | 83.60 | 93.81 | 106.47 | 100.14 |
| WV-42366 | WV-42543 | 29.14 | 42.61 | 35.87 | 68.66 | 51.66 | 60.16 |
| WV-42367 | WV-42544 | 68.59 | 84.38 | 76.48 | 98.97 | 93.58 | 96.27 |
| WV-42368 | WV-42545 | 74.76 | 149.27 | 112.01 | 95.82 | 102.58 | 99.20 |
| WV-42369 | WV-42546 | 49.11 | 63.25 | 56.18 | 75.43 | 66.22 | 70.82 |
| WV-42370 | WV-42547 | 71.07 | 55.73 | 63.40 | 79.73 | 86.04 | 82.88 |
| WV-42371 | WV-42548 | 69.60 | 80.65 | 75.13 | 113.14 | 80.24 | 96.69 |
| WV-42372 | WV-42549 | 43.55 | 67.89 | 55.72 | 87.05 | 76.35 | 81.70 |
| WV-42373 | WV-42550 | 46.72 | 58.04 | 52.38 | 79.63 | 78.67 | 79.15 |
| WV-42374 | WV-42551 | 44.34 | 61.24 | 52.79 | 92.60 | 88.92 | 90.76 |
| WV-42375 | WV-42552 | 61.12 | 55.16 | 58.14 | 88.06 | 98.39 | 93.23 |
| WV-42376 | WV-42553 | 59.98 | 73.55 | 66.76 | 78.30 | 92.07 | 85.18 |
| WV-42377 | WV-42554 | 57.78 | 67.41 | 62.60 | 81.75 | 85.85 | 83.80 |
| WV-42378 | WV-42555 | 79.81 | 80.74 | 80.28 | 93.82 | 81.17 | 87.50 |
| WV-42379 | WV-42556 | 83.26 | 94.81 | 89.04 | 100.41 | 92.05 | 96.23 |
| WV-42380 | WV-42557 | 48.44 | 71.59 | 60.02 | 84.19 | 71.42 | 77.81 |
| WV-42381 | WV-42558 | 22.39 | 29.39 | 25.89 | 52.79 | 56.88 | 54.84 |
| WV-42382 | WV-42559 | 30.88 | 36.73 | 33.80 | 60.99 | 53.12 | 57.06 |
| WV-42383 | WV-42560 | 41.01 | 52.10 | 46.56 | 82.11 | 66.04 | 74.08 |
| WV-42384 | WV-42561 | 90.03 | 110.43 | 100.23 | 97.81 | 78.65 | 88.23 |
| WV-42385 | WV-42562 | 44.13 | 52.21 | 48.17 | 81.90 | 66.79 | 74.35 |
| WV-42386 | WV-42563 | 71.63 | 84.42 | 78.02 | 105.49 | 92.11 | 98.80 |
| WV-42387 | WV-42564 | 71.03 | 79.58 | 75.31 | 84.24 | 71.55 | 77.89 |
| WV-42388 | WV-42565 | 60.79 | 66.39 | 63.59 | 84.32 | 100.05 | 92.18 |
| WV-42389 | WV-42566 | 51.58 | 71.73 | 61.65 | 94.36 | 60.03 | 77.19 |
| WV-42390 | WV-42567 | 55.50 | 60.34 | 57.92 | 80.96 | 65.75 | 73.36 |
| WV-42391 | WV-42568 | 44.36 | 57.01 | 50.68 | 82.64 | 59.38 | 71.01 |
| WV-42392 | WV-42569 | 50.61 | 56.42 | 53.51 | 72.32 | 71.83 | 72.07 |
| WV-42393 | WV-42570 | 39.83 | 64.12 | 51.98 | 79.48 | 68.81 | 74.14 |
| WV-42394 | WV-42571 | 58.38 | 64.52 | 61.45 | 101.64 | 77.35 | 89.50 |
| WV-42395 | WV-42572 | 44.84 | 54.07 | 49.46 | 76.19 | 61.17 | 68.68 |
| WV-42396 | WV-42573 | 67.89 | 69.75 | 68.82 | 77.62 | 60.91 | 69.26 |
| WV-42397 | WV-42574 | 74.96 | 72.18 | 73.57 | 83.87 | 71.84 | 77.85 |

Table 8 shows % human HSD17B13 mRNA remaining (at 300, 100 and 30 nM by gymnotic delivery) relative to human SFRS9 control. N=2. N.D.: Not determined.

TABLE 8

| | | 300 nM | | | 100 nM | | | 30 nM | | |
|---|---|---|---|---|---|---|---|---|---|---|
| | | % remaining | % remaining | | % remaining | % remaining | | % remaining | % remaining | |
| | | mRNA | mRNA | | mRNA | mRNA | | mRNA | mRNA | |
| | | (hHSD17B13/ | (hHSD17B13/ | | (hHSD17B13/ | (hHSD17B13/ | | (hHSD17B13/ | (hHSD17B13/ | |
| Guide | Passenger | hSFRS9)-1 | hSFRS9)-2 | Mean | hSFRS9)-1 | hSFRS9)-2 | Mean | hSFRS9)-1 | hSFRS9)-2 | Mean |
| WV- | WV- | 37.75 | 36.31 | 37.03 | 41.91 | 43.79 | 42.85 | 99.14 | 95.30 | 97.22 |
| 43130 | 43175 | | | | | | | | | |
| WV- | WV- | 56.59 | 44.60 | 50.60 | 61.20 | 56.11 | 58.65 | 99.44 | 105.79 | 102.62 |
| 43131 | 43176 | | | | | | | | | |
| WV- | WV- | 53.84 | 59.77 | 56.80 | 54.25 | 56.05 | 55.15 | 123.03 | 126.48 | 124.75 |
| 43132 | 43177 | | | | | | | | | |
| WV- | WV- | 41.34 | 51.18 | 46.26 | 47.54 | 54.91 | 51.23 | 95.30 | 106.66 | 100.98 |
| 43133 | 43178 | | | | | | | | | |
| WV- | WV- | 21.57 | 31.44 | 26.51 | 28.96 | 55.15 | 42.05 | 71.69 | 84.08 | 77.88 |
| 43134 | 43179 | | | | | | | | | |
| WV- | WV- | 16.87 | 22.20 | 19.54 | 19.23 | 30.69 | 24.96 | 72.25 | 64.11 | 68.18 |
| 43135 | 43180 | | | | | | | | | |
| WV- | WV- | 8.73 | 13.73 | 11.23 | 30.51 | 24.05 | 27.28 | 61.88 | 66.49 | 64.19 |
| 43136 | 43181 | | | | | | | | | |
| WV- | WV- | 31.89 | 35.32 | 33.61 | N.D. | 47.79 | 47.79 | 77.72 | 88.84 | 83.28 |
| 43137 | 43182 | | | | | | | | | |
| WV- | WV- | 30.23 | 29.50 | 29.86 | 32.72 | 35.76 | 34.24 | 70.82 | 66.88 | 68.85 |
| 43138 | 43183 | | | | | | | | | |
| WV- | WV- | 48.61 | 38.02 | 43.31 | 37.10 | 53.79 | 45.45 | 98.63 | 87.24 | 92.94 |
| 43139 | 43184 | | | | | | | | | |
| WV- | WV- | 64.78 | 85.33 | 75.06 | 64.96 | 76.82 | 70.89 | 124.44 | 119.82 | 122.13 |
| 43140 | 43185 | | | | | | | | | |
| WV- | WV- | 69.88 | 75.51 | 72.70 | 73.59 | 72.11 | 72.85 | 113.66 | 146.84 | 130.25 |
| 43141 | 43186 | | | | | | | | | |
| WV- | WV- | 30.71 | 31.19 | 30.95 | 46.99 | 48.01 | 47.50 | 84.46 | 93.87 | 89.16 |
| 43142 | 43187 | | | | | | | | | |
| WV- | WV- | 42.01 | 47.31 | 44.66 | 44.71 | 48.99 | 46.85 | 94.41 | 89.19 | 91.80 |
| 43143 | 43188 | | | | | | | | | |
| WV- | WV- | 48.08 | 45.52 | 46.80 | 59.69 | 53.46 | 56.58 | 106.90 | 123.46 | 115.18 |
| 43144 | 43189 | | | | | | | | | |
| WV- | WV- | 34.13 | 32.85 | 33.49 | 40.37 | 59.48 | 49.92 | 51.18 | 81.86 | 66.52 |
| 43145 | 43190 | | | | | | | | | |
| WV- | WV- | 32.83 | 24.29 | 28.56 | 34.43 | 41.84 | 38.13 | 64.95 | 57.94 | 61.45 |
| 43146 | 43191 | | | | | | | | | |
| WV- | WV- | 37.04 | 37.26 | 37.15 | 40.97 | 44.73 | 42.85 | 80.60 | 82.11 | 81.36 |
| 43147 | 43192 | | | | | | | | | |
| WV- | WV- | 53.75 | 50.53 | 52.14 | 57.70 | 68.78 | 63.24 | 100.57 | 112.76 | 106.66 |
| 43148 | 43193 | | | | | | | | | |
| WV- | WV- | 54.11 | 56.02 | 55.07 | 70.09 | 63.62 | 66.86 | 108.71 | 92.14 | 100.43 |
| 43149 | 43194 | | | | | | | | | |
| WV- | WV- | 47.55 | 48.62 | 48.08 | 62.03 | 58.79 | 60.41 | 70.74 | 107.23 | 88.98 |
| 43150 | 43195 | | | | | | | | | |
| WV- | WV- | 35.13 | 37.62 | 36.38 | 29.52 | 42.59 | 36.05 | 62.44 | 67.77 | 65.11 |
| 43151 | 43196 | | | | | | | | | |
| WV- | WV- | 36.85 | 33.54 | 35.20 | 40.42 | 38.82 | 39.62 | 76.41 | 67.45 | 71.93 |
| 43152 | 43197 | | | | | | | | | |
| WV- | WV- | 27.81 | 35.13 | 31.47 | 37.65 | 36.46 | 37.05 | 70.99 | 51.42 | 61.21 |
| 43153 | 43198 | | | | | | | | | |
| WV- | WV- | 26.02 | 36.76 | 31.39 | 35.23 | 42.20 | 38.72 | 63.18 | 68.71 | 65.95 |
| 43154 | 43199 | | | | | | | | | |
| WV- | WV- | 56.51 | 48.30 | 52.41 | 53.34 | 57.28 | 55.31 | 82.64 | 92.66 | 87.65 |
| 43155 | 43200 | | | | | | | | | |
| WV- | WV- | 63.84 | 80.25 | 72.04 | 68.67 | 72.87 | 70.77 | 102.04 | 90.06 | 96.05 |
| 43156 | 43201 | | | | | | | | | |
| WV- | WV- | 32.70 | 31.77 | 32.23 | 33.54 | 42.09 | 37.82 | 67.36 | 61.70 | 64.53 |
| 43157 | 43202 | | | | | | | | | |
| WV- | WV- | 35.33 | 33.80 | 34.57 | 51.94 | 50.85 | 51.39 | 71.21 | 86.59 | 78.90 |
| 43158 | 43203 | | | | | | | | | |
| WV- | WV- | 60.49 | 67.21 | 63.85 | 81.62 | 72.51 | 77.07 | 93.93 | 112.26 | 103.10 |
| 43159 | 43204 | | | | | | | | | |
| WV- | WV- | 51.18 | 54.79 | 52.99 | 56.30 | 56.22 | 56.26 | 107.26 | 102.13 | 104.69 |
| 43160 | 43205 | | | | | | | | | |
| WV- | WV- | 54.28 | 48.76 | 51.52 | 60.05 | 61.90 | 60.98 | 100.55 | 94.99 | 97.77 |
| 43161 | 43206 | | | | | | | | | |
| WV- | WV- | 49.18 | 57.70 | 53.44 | 59.64 | 55.43 | 57.54 | 79.45 | 90.82 | 85.14 |
| 43162 | 43207 | | | | | | | | | |
| WV- | WV- | 31.34 | 35.61 | 33.47 | 55.35 | 60.65 | 58.00 | 78.51 | 85.56 | 82.04 |
| 43163 | 43208 | | | | | | | | | |
| WV- | WV- | 35.02 | 30.83 | 32.92 | 84.78 | 55.27 | 70.02 | 79.63 | 80.45 | 80.04 |
| 43164 | 43209 | | | | | | | | | |
| WV- | WV- | 62.40 | 68.19 | 65.29 | 74.40 | 81.15 | 77.78 | 96.77 | 96.93 | 96.85 |
| 43165 | 43210 | | | | | | | | | |
| WV- | WV- | 47.14 | 45.94 | 46.54 | 46.06 | 56.96 | 51.51 | 76.18 | 86.85 | 81.51 |
| 43166 | 43211 | | | | | | | | | |
| WV- | WV- | 34.59 | 44.70 | 39.64 | 41.94 | 44.44 | 43.19 | 64.97 | 64.75 | 64.86 |
| 43167 | 43212 | | | | | | | | | |
| WV- | WV- | 58.33 | 45.14 | 51.73 | 63.46 | 55.40 | 59.43 | 89.42 | 102.86 | 96.14 |
| 43168 | 43213 | | | | | | | | | |
| WV- | WV- | 40.36 | 54.62 | 47.49 | 59.95 | 63.63 | 61.79 | 95.54 | 93.76 | 94.65 |
| 43169 | 43214 | | | | | | | | | |
| WV- | WV- | 37.59 | 43.23 | 40.41 | 43.08 | 52.61 | 47.84 | 79.30 | 71.64 | 75.47 |
| 43170 | 43215 | | | | | | | | | |
| WV- | WV- | 32.92 | 27.25 | 30.08 | 42.62 | 37.38 | 40.00 | 56.41 | 82.36 | 69.38 |
| 43171 | 43216 | | | | | | | | | |
| WV- | WV- | 35.11 | 35.64 | 35.37 | 55.87 | 63.25 | 59.56 | N.D. | 77.93 | 77.93 |
| 43172 | 43217 | | | | | | | | | |
| WV- | WV- | 38.49 | 33.33 | 35.91 | 45.44 | 45.35 | 45.40 | 90.02 | 60.12 | 75.07 |
| 43173 | 43218 | | | | | | | | | |
| WV- | WV- | 36.48 | 40.15 | 38.31 | 50.67 | 51.20 | 50.94 | 72.33 | 76.71 | 74.52 |
| 43174 | 43219 | | | | | | | | | |

Table 9 shows % NHP HSD17B13 mRNA remaining (at 150 and 50 nM by gymnotic delivery) relative to NHP SFRS9 control. N=2. N.D.: Not determined.

TABLE 9

| | | 150 nM | | | 50 nM | | |
|---|---|---|---|---|---|---|---|
| | | % remaining | % remaining | | % remaining | % remaining | |
| | | mRNA | mRNA | | mRNA | mRNA | |
| | | (hHSD17B13/ | (hHSD17B13/ | | (hHSD17B13/ | (hHSD17B13/ | |
| Guide | Passenger | hSFRS9)-1 | hSFRS9)-2 | Mean | hSFRS9)-1 | hSFRS9)-2 | Mean |
| WV-43130 | WV-43175 | 67.45 | 70.16 | 68.80 | 44.51 | 66.47 | 55.49 |
| WV-43131 | WV-43176 | 84.31 | 106.44 | 95.37 | 60.98 | 107.62 | 84.30 |
| WV-43132 | WV-43177 | 64.37 | 73.76 | 69.07 | 87.59 | 89.67 | 88.63 |
| WV-43133 | WV-43178 | 61.69 | 63.06 | 62.37 | 73.40 | 73.43 | 73.42 |
| WV-43134 | WV-43179 | 41.59 | 48.80 | 45.19 | 59.98 | 63.14 | 61.56 |
| WV-43135 | WV-43180 | 44.68 | 32.98 | 38.83 | 34.87 | 56.19 | 45.53 |
| WV-43136 | WV-43181 | 18.37 | 22.23 | 20.30 | 22.36 | N.D. | 22.36 |
| WV-43137 | WV-43182 | 40.09 | 39.18 | 39.63 | 150.58 | 90.21 | 120.39 |
| WV-43138 | WV-43183 | 36.51 | 34.70 | 35.60 | 26.07 | 39.92 | 32.99 |
| WV-43139 | WV-43184 | 61.49 | 55.75 | 58.62 | 51.74 | 57.01 | 54.37 |
| WV-43140 | WV-43185 | 66.49 | 77.86 | 72.17 | 62.90 | 57.24 | 60.07 |
| WV-43141 | WV-43186 | 53.79 | 67.42 | 60.60 | 77.08 | 75.37 | 76.22 |
| WV-43142 | WV-43187 | 33.83 | 39.66 | 36.75 | 56.67 | 29.38 | 43.02 |
| WV-43143 | WV-43188 | 27.74 | 40.89 | 34.31 | 67.39 | 100.71 | 84.05 |
| WV-43144 | WV-43189 | 45.90 | 50.99 | 48.44 | 105.08 | 75.84 | 90.46 |
| WV-43145 | WV-43190 | 32.55 | 35.35 | 33.95 | 49.55 | 98.64 | 74.09 |
| WV-43146 | WV-43191 | 14.16 | 77.69 | 45.93 | 9.79 | 22.50 | 16.14 |
| WV-43147 | WV-43192 | 30.93 | 37.76 | 34.34 | 24.76 | 23.03 | 23.89 |
| WV-43148 | WV-43193 | 60.78 | 104.31 | 82.55 | 47.44 | 56.14 | 51.79 |
| WV-43149 | WV-43194 | 57.53 | 70.12 | 63.83 | 53.74 | 71.30 | 62.52 |
| WV-43150 | WV-43195 | 37.95 | 45.96 | 41.96 | 49.10 | 63.03 | 56.06 |
| WV-43151 | WV-43196 | 27.73 | 30.07 | 28.90 | 35.21 | 45.44 | 40.32 |
| WV-43152 | WV-43197 | 25.75 | 26.85 | 26.30 | 51.93 | 53.80 | 52.86 |
| WV-43153 | WV-43198 | 27.39 | 42.46 | 34.92 | 35.08 | 34.62 | 34.85 |
| WV-43154 | WV-43199 | 20.51 | 33.18 | 26.85 | 19.89 | 12.30 | 16.10 |
| WV-43155 | WV-43200 | 30.91 | 43.49 | 37.20 | 31.72 | 76.64 | 54.18 |
| WV-43156 | WV-43201 | 68.02 | 63.95 | 65.98 | 51.99 | 69.16 | 60.58 |
| WV-43157 | WV-43202 | 31.65 | 24.28 | 27.97 | 36.44 | 38.72 | 37.58 |
| WV-43158 | WV-43203 | 23.71 | 43.15 | 33.43 | 40.56 | 44.76 | 42.66 |
| WV-43159 | WV-43204 | 86.99 | 87.18 | 87.08 | 85.71 | 86.43 | 86.07 |
| WV-43160 | WV-43205 | 32.73 | 44.91 | 38.82 | 53.69 | 65.36 | 59.52 |
| WV-43161 | WV-43206 | 48.56 | 63.15 | 55.85 | 87.77 | 70.86 | 79.31 |
| WV-43162 | WV-43207 | 69.99 | 40.43 | 55.21 | 37.16 | 28.81 | 32.98 |
| WV-43163 | WV-43208 | 53.85 | 65.71 | 59.78 | 36.81 | 41.42 | 39.12 |
| WV-43164 | WV-43209 | 27.77 | 36.72 | 32.25 | 33.06 | 20.96 | 27.01 |
| WV-43165 | WV-43210 | 51.26 | 55.54 | 53.40 | 95.64 | 76.58 | 86.11 |
| WV-43166 | WV-43211 | 32.62 | 28.56 | 30.59 | 49.54 | 44.57 | 47.05 |
| WV-43167 | WV-43212 | 38.05 | 40.15 | 39.10 | 42.27 | 49.51 | 45.89 |
| WV-43168 | WV-43213 | 45.28 | 42.59 | 43.94 | 68.83 | 65.91 | 67.37 |
| WV-43169 | WV-43214 | 68.18 | 52.74 | 60.46 | 92.26 | 60.88 | 76.57 |
| WV-43170 | WV-43215 | 47.08 | 37.54 | 42.31 | 26.27 | 26.85 | 26.56 |
| WV-43171 | WV-43216 | 23.91 | 40.95 | 32.43 | 16.07 | 26.40 | 21.23 |
| WV-43172 | WV-43217 | 39.16 | 63.58 | 51.37 | 51.90 | 78.95 | 65.42 |
| WV-43173 | WV-43218 | 62.11 | 80.55 | 71.33 | 59.56 | 48.87 | 54.21 |
| WV-43174 | WV-43219 | 51.10 | 36.83 | 43.96 | 44.63 | 47.03 | 45.83 |

Table 10 shows % human HSD17B13 mRNA remaining (at 300, 100 and 30 nM by gymnotic delivery) relative to human SFRS9 control. N=2. N.D.: Not determined.

TABLE 10

| | | 300 nM | | | 100 nM | | | 30 nM | | |
|---|---|---|---|---|---|---|---|---|---|---|
| | | % remaining | % remaining | | % remaining | % remaining | | % remaining | % remaining | |
| | | mRNA | mRNA | | mRNA | mRNA | | mRNA | mRNA | |
| | | (hHSD17B13/ | (hHSD17B13/ | | (hHSD17B13/ | (hHSD17B13/ | | (hHSD17B13/ | (hHSD17B13/ | |
| Guide | Passenger | hSFRS9)-1 | hSFRS9)-2 | Mean | hSFRS9)-1 | hSFRS9)-2 | Mean | hSFRS9)-1 | hSFRS9)-2 | Mean |
| WV- | WV- | 40.50 | 36.28 | 38.39 | 65.94 | 73.42 | 69.68 | 96.15 | 65.15 | 80.65 |
| 42354 | 42531 | | | | | | | | | |
| WV- | WV- | 27.83 | 64.63 | 46.23 | 57.85 | 58.01 | 57.93 | 48.93 | 74.06 | 61.50 |
| 42355 | 42532 | | | | | | | | | |
| WV- | WV- | 29.10 | 30.71 | 29.91 | 43.63 | 32.91 | 38.27 | 65.74 | 57.29 | 61.51 |
| 42356 | 42533 | | | | | | | | | |
| WV- | WV- | 42.92 | 56.52 | 49.72 | 50.82 | 49.59 | 50.21 | 54.41 | 48.14 | 51.28 |
| 42357 | 42534 | | | | | | | | | |
| WV- | WV- | 48.17 | 55.00 | 51.59 | 57.86 | 59.63 | 58.75 | 38.57 | 43.38 | 40.97 |
| 42358 | 42535 | | | | | | | | | |
| WV- | WV- | 44.42 | 50.59 | 47.51 | 58.39 | 66.85 | 62.62 | 79.24 | 50.91 | 65.08 |
| 42359 | 42536 | | | | | | | | | |
| WV- | WV- | 37.87 | 42.81 | 40.34 | 58.31 | 36.62 | 47.46 | 90.89 | 67.38 | 79.13 |
| 42361 | 42538 | | | | | | | | | |
| WV- | WV- | 59.19 | 26.39 | 42.79 | N.D. | 43.95 | 43.95 | 58.61 | 57.74 | 58.17 |
| 42362 | 42539 | | | | | | | | | |
| WV- | WV- | 38.38 | 25.99 | 32.19 | 35.66 | 62.06 | 48.86 | 93.74 | 66.15 | 79.94 |
| 42363 | 42540 | | | | | | | | | |
| WV- | WV- | 49.50 | 29.44 | 39.47 | 48.63 | 57.90 | 53.26 | 38.35 | 48.48 | 43.41 |
| 42364 | 42541 | | | | | | | | | |
| WV- | WV- | 33.63 | 41.32 | 37.47 | 33.46 | 42.88 | 38.17 | 61.26 | 56.38 | 58.82 |
| 42366 | 42543 | | | | | | | | | |
| WV- | WV- | 25.34 | 33.23 | 29.28 | 49.54 | 66.37 | 57.96 | 65.06 | 68.56 | 66.81 |
| 42369 | 42546 | | | | | | | | | |
| WV- | WV- | 25.79 | 44.05 | 34.92 | 22.22 | 37.33 | 29.78 | 61.43 | 53.15 | 57.29 |
| 42373 | 42550 | | | | | | | | | |
| WV- | WV- | 18.72 | 12.94 | 15.83 | 26.64 | 47.49 | 37.06 | 43.36 | 49.69 | 46.52 |
| 42381 | 42558 | | | | | | | | | |
| WV- | WV- | 13.68 | 14.14 | 13.91 | 28.70 | 29.25 | 28.97 | 95.25 | 63.83 | 79.54 |
| 42382 | 42559 | | | | | | | | | |
| WV- | WV- | 21.11 | 12.32 | 16.71 | 104.70 | 39.11 | 71.90 | 47.95 | 35.04 | 41.50 |
| 42383 | 42560 | | | | | | | | | |
| WV- | WV- | 23.55 | 90.33 | 56.94 | 69.16 | 76.67 | 72.91 | 80.26 | 75.16 | 77.71 |
| 42385 | 42562 | | | | | | | | | |
| WV- | WV- | 67.25 | 51.32 | 59.28 | 51.50 | 80.98 | 66.24 | 78.68 | 68.34 | 73.51 |
| 42390 | 42567 | | | | | | | | | |
| WV- | WV- | 62.78 | 38.05 | 50.41 | 56.99 | 59.76 | 58.37 | 57.21 | 48.19 | 52.70 |
| 42391 | 42568 | | | | | | | | | |
| WV- | WV- | 38.67 | 27.40 | 33.04 | 54.19 | 39.75 | 46.97 | 44.31 | 75.77 | 60.04 |
| 42392 | 42569 | | | | | | | | | |
| WV- | WV- | 50.33 | 40.21 | 45.27 | 55.00 | 52.86 | 53.93 | 100.54 | 109.76 | 105.15 |
| 42393 | 42570 | | | | | | | | | |
| WV- | WV- | 43.11 | 32.82 | 37.97 | 60.12 | 47.32 | 53.72 | 62.18 | 66.81 | 64.50 |
| 42395 | 42572 | | | | | | | | | |
| WV- | WV- | 55.52 | 48.97 | 52.24 | 77.55 | 58.80 | 68.18 | 69.70 | 83.08 | 76.39 |
| 42396 | 42573 | | | | | | | | | |
| WV- | WV- | 59.30 | 41.45 | 50.38 | 35.57 | 40.01 | 37.79 | 62.29 | 73.79 | 68.04 |
| 43135 | 43180 | | | | | | | | | |
| WV- | WV- | 50.21 | 25.44 | 37.82 | 41.05 | 29.71 | 35.38 | 67.29 | 58.34 | 62.81 |
| 43136 | 43181 | | | | | | | | | |
| WV- | WV- | 85.37 | 53.15 | 69.26 | 61.84 | 57.36 | 59.60 | 77.63 | 71.30 | 74.46 |
| 43137 | 43182 | | | | | | | | | |
| WV- | WV- | 39.18 | 54.65 | 46.91 | 54.03 | 73.16 | 63.59 | 87.73 | 88.87 | 88.30 |
| 43138 | 43183 | | | | | | | | | |
| WV- | WV- | 62.43 | 68.92 | 65.68 | 70.35 | 86.71 | 78.53 | 68.50 | 104.64 | 86.57 |
| 43143 | 43188 | | | | | | | | | |
| WV- | WV- | 63.30 | 73.98 | 68.64 | 75.39 | 70.76 | 73.07 | 86.66 | 110.26 | 98.46 |
| 43145 | 43190 | | | | | | | | | |
| WV- | WV- | 44.44 | 51.65 | 48.04 | 59.91 | 56.48 | 58.19 | 66.02 | 81.43 | 73.72 |
| 43146 | 43191 | | | | | | | | | |
| WV- | WV- | 40.18 | 67.98 | 54.08 | 92.75 | 209.73 | 151.24 | 70.31 | 90.23 | 80.27 |
| 43147 | 43192 | | | | | | | | | |
| WV- | WV- | 61.50 | 51.94 | 56.72 | 50.31 | 52.78 | 51.54 | 64.61 | 62.99 | 63.80 |
| 43151 | 43196 | | | | | | | | | |
| WV- | WV- | 57.02 | 84.93 | 70.97 | 65.53 | 52.07 | 58.80 | 80.93 | 69.17 | 75.05 |
| 43152 | 43197 | | | | | | | | | |
| WV- | WV- | 61.48 | 79.92 | 70.70 | 65.67 | 73.11 | 69.39 | 84.22 | 59.23 | 71.72 |
| 43153 | 43198 | | | | | | | | | |
| WV- | WV- | 40.59 | 22.09 | 31.34 | 60.04 | 74.99 | 67.52 | 77.17 | 69.70 | 73.44 |
| 43154 | 43199 | | | | | | | | | |
| WV- | WV- | 47.49 | 80.49 | 63.99 | 81.73 | 64.53 | 73.13 | 81.59 | 146.21 | 113.90 |
| 43155 | 43200 | | | | | | | | | |
| WV- | WV- | 40.39 | 46.32 | 43.36 | 73.92 | 68.94 | 71.43 | 47.23 | 90.03 | 68.63 |
| 43157 | 43202 | | | | | | | | | |
| WV- | WV- | 35.95 | 34.02 | 34.98 | 90.00 | 82.83 | 86.41 | 59.51 | 53.94 | 56.73 |
| 43158 | 43203 | | | | | | | | | |
| WV- | WV- | 52.82 | 20.26 | 36.54 | 78.60 | 76.69 | 77.64 | 84.27 | 70.03 | 77.15 |
| 43164 | 43209 | | | | | | | | | |
| WV- | WV- | 73.23 | 52.70 | 62.97 | 79.02 | 63.73 | 71.37 | 109.46 | 100.94 | 105.20 |
| 43166 | 43211 | | | | | | | | | |
| WV- | WV- | 45.11 | 44.82 | 44.97 | 65.99 | 84.00 | 74.99 | 58.16 | 76.80 | 67.48 |
| 43167 | 43212 | | | | | | | | | |
| WV- | WV- | 26.59 | 111.76 | 69.17 | 79.65 | 87.61 | 83.63 | 67.67 | 77.77 | 72.72 |
| 43170 | 43215 | | | | | | | | | |
| WV- | WV- | 43.95 | 43.77 | 43.86 | 59.32 | 59.44 | 59.38 | 60.49 | 61.58 | 61.04 |
| 43171 | 43216 | | | | | | | | | |
| WV- | WV- | 54.55 | 41.37 | 47.96 | 110.49 | 63.83 | 87.16 | 77.80 | 80.23 | 79.01 |
| 43173 | 43218 | | | | | | | | | |
| WV- | WV- | 65.61 | 49.44 | 57.53 | 75.97 | 93.54 | 84.76 | 74.59 | 60.57 | 67.58 |
| 43174 | 43219 | | | | | | | | | |

Table 11 shows % NHP HSD17B13 mRNA remaining (at 150 and 50 nM by gymnotic delivery) relative to NHP SFRS9 control. N=2. N.D.: Not determined.

TABLE 11

| | | 150 nM | | | 50 nM | | |
|---|---|---|---|---|---|---|---|
| | | % remaining | % remaining | | % remaining | % remaining | |
| | | mRNA | mRNA | | mRNA | mRNA | |
| | | (hHSD17B13/ | (hHSD17B13/ | | (hHSD17B13/ | (hHSD17B13/ | |
| Guide | Passenger | hSFRS9)-1 | hSFRS9)-2 | Mean | hSFRS9)-1 | hSFRS9)-2 | Mean |
| WV-42354 | WV-42531 | 76.61 | 67.83 | 72.22 | 63.55 | 74.57 | 69.06 |
| WV-42355 | WV-42532 | 102.41 | 106.81 | 104.61 | 60.47 | 77.02 | 68.75 |
| WV-42356 | WV-42533 | 83.06 | 68.00 | 75.53 | 49.89 | 61.39 | 55.64 |
| WV-42357 | WV-42534 | 110.08 | 68.25 | 89.16 | 55.14 | 52.48 | 53.81 |
| WV-42358 | WV-42535 | 66.17 | 58.38 | 62.28 | 35.08 | 67.75 | 51.41 |
| WV-42359 | WV-42536 | 74.02 | 84.76 | 79.39 | 39.73 | 72.61 | 56.17 |
| WV-42361 | WV-42538 | 47.38 | 35.25 | 41.32 | 41.80 | 60.34 | 51.07 |
| WV-42362 | WV-42539 | 30.13 | 46.20 | 38.16 | 83.28 | 38.44 | 60.86 |
| WV-42363 | WV-42540 | 62.99 | 80.90 | 71.95 | 59.18 | 70.27 | 64.72 |
| WV-42364 | WV-42541 | 140.29 | 103.94 | 122.11 | 83.77 | 47.99 | 65.88 |
| WV-42366 | WV-42543 | 111.51 | 117.36 | 114.44 | 75.31 | 64.52 | 69.92 |
| WV-42369 | WV-42546 | 122.98 | 106.49 | 114.74 | 94.05 | 80.00 | 87.02 |
| WV-42373 | WV-42550 | N.D. | 101.56 | 101.56 | 68.19 | 72.61 | 70.40 |
| WV-42381 | WV-42558 | 58.45 | 44.25 | 51.35 | 78.70 | 49.40 | 64.05 |
| WV-42382 | WV-42559 | 64.41 | N.D. | 64.41 | 93.66 | 101.96 | 97.81 |
| WV-42383 | WV-42560 | 56.09 | 66.90 | 61.49 | 56.93 | 67.10 | 62.01 |
| WV-42385 | WV-42562 | 95.13 | 71.61 | 83.37 | 69.92 | 60.61 | 65.27 |
| WV-42390 | WV-42567 | 76.84 | 109.21 | 93.02 | 56.85 | 62.87 | 59.86 |
| WV-42391 | WV-42568 | 90.96 | 73.44 | 82.20 | 61.63 | 98.78 | 80.20 |
| WV-42392 | WV-42569 | 95.56 | 102.72 | 99.14 | 74.47 | 104.28 | 89.37 |
| WV-42393 | WV-42570 | 89.02 | 81.11 | 85.07 | 78.42 | 66.07 | 72.25 |
| WV-42395 | WV-42572 | 74.58 | 82.94 | 78.76 | 87.14 | 101.09 | 94.11 |
| WV-42396 | WV-42573 | 62.53 | 72.66 | 67.59 | 128.48 | 58.06 | 93.27 |
| WV-43135 | WV-43180 | 49.54 | 76.19 | 62.86 | 59.76 | 104.32 | 82.04 |
| WV-43136 | WV-43181 | 32.72 | 59.37 | 46.04 | 68.46 | 60.91 | 64.68 |
| WV-43137 | WV-43182 | 141.43 | 74.06 | 107.75 | 84.20 | 92.34 | 88.27 |
| WV-43138 | WV-43183 | 82.75 | 89.46 | 86.11 | 204.83 | 66.73 | 135.78 |
| WV-43143 | WV-43188 | 105.97 | 93.49 | 99.73 | 100.13 | 73.97 | 87.05 |
| WV-43145 | WV-43190 | 69.39 | 66.33 | 67.86 | 94.60 | 76.97 | 85.78 |
| WV-43146 | WV-43191 | 78.63 | 84.23 | 81.43 | 81.05 | 107.19 | 94.12 |
| WV-43147 | WV-43192 | 75.11 | 58.60 | 66.85 | 62.51 | 98.21 | 80.36 |
| WV-43151 | WV-43196 | 41.76 | 69.95 | 55.85 | 64.64 | 73.52 | 69.08 |
| WV-43152 | WV-43197 | 51.95 | 49.71 | 50.83 | 51.77 | 69.35 | 60.56 |
| WV-43153 | WV-43198 | 69.62 | 91.77 | 80.69 | 84.12 | 71.61 | 77.87 |
| WV-43154 | WV-43199 | 41.56 | 88.66 | 65.11 | 44.71 | 50.31 | 47.51 |
| WV-43155 | WV-43200 | 84.84 | 150.94 | 117.89 | 72.78 | 97.87 | 85.33 |
| WV-43157 | WV-43202 | 52.73 | 66.08 | 59.40 | 75.03 | 82.74 | 78.88 |
| WV-43158 | WV-43203 | 59.84 | 81.64 | 70.74 | 130.73 | 75.19 | 102.96 |
| WV-43164 | WV-43209 | 55.08 | 83.98 | 69.53 | 68.35 | 62.77 | 65.56 |
| WV-43166 | WV-43211 | 84.37 | 46.69 | 65.53 | 87.74 | 77.83 | 82.79 |
| WV-43167 | WV-43212 | 56.78 | 80.08 | 68.43 | 69.64 | 81.65 | 75.64 |
| WV-43170 | WV-43215 | 103.48 | 65.24 | 84.36 | 72.07 | 80.22 | 76.14 |
| WV-43171 | WV-43216 | 126.59 | 45.37 | 85.98 | 78.15 | 62.22 | 70.19 |
| WV-43173 | WV-43218 | 107.94 | 83.95 | 95.95 | 91.75 | 91.61 | 91.68 |
| WV-43174 | WV-43219 | 84.45 | 49.79 | 67.12 | 59.68 | 109.00 | 84.34 |

Table 12 shows % human HSD17B13 miRNA remaining (at 500, 50 and 5 nM by gymnotic delivery) relative to human SFRS9 control. N=2. N.D.: Not determined.

TABLE 12

| | | 500 nM | | | 50 nM | | | 5 nM | | |
|---|---|---|---|---|---|---|---|---|---|---|
| | | % remaining | % remaining | | % remaining | % remaining | | % remaining | % remaining | |
| | | mRNA | mRNA | | mRNA | mRNA | | mRNA | mRNA | |
| | | (hHSD17B13/ | (hHSD17B13/ | | (hHSD17B13/ | (hHSD17B13/ | | (hHSD17B13/ | (hHSD17B13/ | |
| Guide | Passenger | hSFRS9)-1 | hSFRS9)-2 | Mean | hSFRS9)-1 | hSFRS9)-2 | Mean | hSFRS9)-1 | hSFRS9)-2 | Mean |
| WV- | WV- | 10.95 | 11.14 | 11.04 | 26.95 | 32.48 | 29.71 | 53.74 | 67.81 | 60.77 |
| 43135 | 44173 | | | | | | | | | |
| WV- | WV- | 10.23 | 11.80 | 11.02 | 32.98 | 29.84 | 31.41 | 63.38 | 78.00 | 70.69 |
| 42373 | 42417 | | | | | | | | | |
| WV- | WV- | 8.17 | 21.36 | 14.77 | 24.53 | 25.89 | 25.21 | 51.21 | 64.43 | 57.82 |
| 45021 | 42588 | | | | | | | | | |
| WV- | WV- | 6.51 | 10.44 | 8.48 | 27.74 | 24.48 | 26.11 | 84.92 | 82.72 | 83.82 |
| 45022 | 42588 | | | | | | | | | |
| WV- | WV- | 8.73 | 14.72 | 11.72 | 34.35 | 36.20 | 35.27 | 63.42 | 69.86 | 66.64 |
| 45023 | 42588 | | | | | | | | | |
| WV- | WV- | 32.55 | 36.47 | 34.51 | 47.47 | 72.67 | 60.07 | 77.43 | 107.66 | 92.54 |
| 45024 | 42588 | | | | | | | | | |
| WV- | WV- | 13.08 | 20.28 | 16.68 | 41.04 | 41.90 | 41.47 | 57.27 | 77.45 | 67.36 |
| 45025 | 42588 | | | | | | | | | |
| WV- | WV- | 39.92 | 47.24 | 43.58 | 67.56 | 67.89 | 67.73 | 95.27 | 96.26 | 95.77 |
| 45026 | 42588 | | | | | | | | | |
| WV- | WV- | 8.97 | 11.60 | 10.28 | 30.71 | 38.06 | 34.38 | 76.32 | 74.23 | 75.27 |
| 45027 | 42588 | | | | | | | | | |
| WV- | WV- | 37.40 | 46.69 | 42.04 | 67.19 | 74.09 | 70.64 | 90.20 | 86.53 | 88.36 |
| 45028 | 42588 | | | | | | | | | |
| WV- | WV- | 11.89 | 27.35 | 19.62 | 27.77 | 36.28 | 32.02 | 76.85 | 79.32 | 78.08 |
| 45029 | 42588 | | | | | | | | | |
| WV- | WV- | 19.68 | 23.86 | 21.77 | 49.16 | 60.16 | 54.66 | 89.85 | 87.10 | 88.48 |
| 45030 | 42588 | | | | | | | | | |
| WV- | WV- | 3.42 | 5.24 | 4.33 | 12.75 | 17.51 | 15.13 | 47.45 | 52.25 | 49.85 |
| 45031 | 42588 | | | | | | | | | |
| WV- | WV- | 5.03 | 6.18 | 5.61 | 16.17 | 14.69 | 15.43 | 59.80 | 66.68 | 63.24 |
| 45032 | 42588 | | | | | | | | | |
| WV- | WV- | 3.73 | 13.22 | 8.47 | 30.22 | 23.84 | 27.03 | 62.68 | 61.76 | 62.22 |
| 45033 | 42588 | | | | | | | | | |
| WV- | WV- | 19.85 | 35.67 | 27.76 | 51.95 | 54.05 | 53.00 | 102.04 | 97.34 | 99.69 |
| 45034 | 42588 | | | | | | | | | |
| WV- | WV- | 8.65 | 8.64 | 8.65 | 24.26 | 31.61 | 27.94 | 77.18 | 63.47 | 70.33 |
| 45035 | 42588 | | | | | | | | | |
| WV- | WV- | 24.84 | 33.65 | 29.25 | 61.72 | 65.48 | 63.60 | 89.55 | 83.07 | 86.31 |
| 45036 | 42588 | | | | | | | | | |
| WV- | WV- | 7.99 | 6.39 | 7.19 | 26.11 | 20.39 | 23.25 | 62.74 | 94.80 | 78.77 |
| 45037 | 42588 | | | | | | | | | |
| WV- | WV- | 31.74 | 27.94 | 29.84 | 64.14 | 55.89 | 60.01 | 100.93 | 100.49 | 100.71 |
| 45038 | 42588 | | | | | | | | | |
| WV- | WV- | 10.74 | 12.57 | 11.66 | 26.79 | 30.34 | 28.57 | 64.10 | 60.78 | 62.44 |
| 45039 | 42588 | | | | | | | | | |
| WV- | WV- | 39.87 | 35.06 | 37.47 | 61.65 | 76.68 | 69.16 | 104.61 | 96.85 | 100.73 |
| 45040 | 42588 | | | | | | | | | |
| WV- | WV- | 3.48 | 3.67 | 3.58 | 21.67 | 23.26 | 22.46 | 59.50 | 64.74 | 62.12 |
| 42383 | 42427 | | | | | | | | | |
| WV- | WV- | 4.67 | 3.49 | 4.08 | 15.08 | 14.10 | 14.59 | 66.94 | 65.59 | 66.27 |
| 45041 | 42589 | | | | | | | | | |
| WV- | WV- | 2.26 | 6.06 | 4.16 | 12.98 | 14.99 | 13.98 | 57.41 | 53.71 | 55.56 |
| 45042 | 42589 | | | | | | | | | |
| WV- | WV- | 10.21 | 13.10 | 11.66 | 34.18 | 30.81 | 32.50 | 80.73 | 91.69 | 86.21 |
| 45043 | 42589 | | | | | | | | | |
| WV- | WV- | 24.59 | 22.95 | 23.77 | 56.42 | 56.29 | 56.35 | 81.01 | 80.82 | 80.91 |
| 45044 | 42589 | | | | | | | | | |
| WV- | WV- | 12.47 | 11.25 | 11.86 | 42.24 | 33.67 | 37.96 | 75.13 | 91.44 | 83.28 |
| 45045 | 42589 | | | | | | | | | |
| WV- | WV- | 21.44 | 26.01 | 23.72 | 70.63 | 66.31 | 68.47 | 78.58 | 94.66 | 86.62 |
| 45046 | 42589 | | | | | | | | | |
| WV- | WV- | 13.10 | 8.86 | 10.98 | 36.51 | 41.27 | 38.89 | 91.81 | 83.46 | 87.64 |
| 45047 | 42589 | | | | | | | | | |
| WV- | WV- | 30.58 | 30.99 | 30.78 | 58.82 | 63.92 | 61.37 | 81.12 | 86.25 | 83.68 |
| 45048 | 42589 | | | | | | | | | |
| WV- | WV- | 14.61 | 16.59 | 15.60 | 40.85 | 45.61 | 43.23 | 79.81 | 80.26 | 80.03 |
| 45049 | 42589 | | | | | | | | | |
| WV- | WV- | 34.06 | 24.36 | 29.21 | 54.47 | 60.20 | 57.33 | 90.11 | 89.59 | 89.85 |
| 45050 | 42589 | | | | | | | | | |
| WV- | WV- | 5.81 | 1.38 | 3.60 | 10.43 | 13.17 | 11.80 | 60.27 | 49.32 | 54.80 |
| 45051 | 42589 | | | | | | | | | |
| WV- | WV- | 4.16 | 3.71 | 3.93 | 5.30 | 10.16 | 7.73 | 38.13 | 41.91 | 40.02 |
| 45052 | 42589 | | | | | | | | | |
| WV- | WV- | 6.67 | 8.20 | 7.44 | 31.50 | 30.58 | 31.04 | 80.37 | 72.35 | 76.36 |
| 45053 | 42589 | | | | | | | | | |
| WV- | WV- | 22.92 | 15.45 | 19.18 | 48.21 | 43.42 | 45.81 | 89.91 | 85.25 | 87.58 |
| 45054 | 42589 | | | | | | | | | |
| WV- | WV- | 9.76 | 11.35 | 10.56 | 32.60 | 27.51 | 30.06 | 104.19 | 69.19 | 86.69 |
| 45055 | 42589 | | | | | | | | | |
| WV- | WV- | 32.08 | 15.17 | 23.63 | 54.42 | 53.20 | 53.81 | 82.41 | 82.02 | 82.22 |
| 45056 | 42589 | | | | | | | | | |
| WV- | WV- | 4.50 | 14.02 | 9.26 | 25.82 | 29.32 | 27.57 | 69.15 | 67.37 | 68.26 |
| 45057 | 42589 | | | | | | | | | |
| WV- | WV- | 21.41 | 19.99 | 20.70 | 48.40 | 43.09 | 45.74 | 91.20 | 110.39 | 100.79 |
| 45058 | 42589 | | | | | | | | | |
| WV- | WV- | 10.32 | 17.39 | 13.86 | 25.85 | 27.12 | 26.48 | 90.20 | 75.64 | 82.92 |
| 45059 | 42589 | | | | | | | | | |
| WV- | WV- | 40.77 | 26.84 | 33.81 | 45.71 | 50.73 | 48.22 | 89.90 | 78.25 | 84.08 |
| 45060 | 42589 | | | | | | | | | |

Table 13 shows % human HSD17B13 mRNA remaining (at 15, 3 and 0.6 nM by gymnotic delivery) relative to human SFRS9 control. N=2. N.D.: Not determined.

TABLE 13

| | | 15 nM | | | 3 nM | | | 0.6 nM | | |
|---|---|---|---|---|---|---|---|---|---|---|
| | | % remaining | % remaining | | % remaining | % remaining | | % remaining | % remaining | |
| | | mRNA | mRNA | | mRNA | mRNA | | mRNA | mRNA | |
| | | (hHSD17B13/ | (hHSD17B13/ | | (hHSD17B13/ | (hHSD17B13/ | | (hHSD17B13/ | (hHSD17B13/ | |
| Guide | Passenger | hSFRS9)-1 | hSFRS9)-2 | Mean | hSFRS9)-1 | hSFRS9)-2 | Mean | hSFRS9)-1 | hSFRS9)-2 | Mean |
| WV- | WV- | 14.92 | 20.01 | 17.47 | 30.78 | 27.40 | 29.09 | 39.02 | 48.66 | 43.84 |
| 43135 | 44173 | | | | | | | | | |
| WV- | WV- | 13.45 | 22.31 | 17.88 | 31.32 | 42.52 | 36.92 | 56.18 | 50.43 | 53.31 |
| 42373 | 42417 | | | | | | | | | |
| WV- | WV- | 17.46 | 15.97 | 16.72 | 43.36 | 21.65 | 32.51 | 44.01 | 49.00 | 46.50 |
| 45021 | 42588 | | | | | | | | | |
| WV- | WV- | 16.99 | 15.08 | 16.03 | 34.01 | 37.54 | 35.78 | 60.59 | 45.90 | 53.25 |
| 46471 | 42588 | | | | | | | | | |
| WV- | WV- | 11.93 | 18.97 | 15.45 | 40.43 | 40.72 | 40.57 | 38.82 | 39.42 | 39.12 |
| 46472 | 42588 | | | | | | | | | |
| WV- | WV- | 18.67 | 18.37 | 18.52 | 29.72 | 45.75 | 37.73 | 53.59 | 48.59 | 51.09 |
| 46473 | 42588 | | | | | | | | | |
| WV- | WV- | 12.27 | 13.40 | 12.83 | 54.38 | 27.21 | 40.80 | 45.11 | 45.84 | 45.47 |
| 46474 | 42588 | | | | | | | | | |
| WV- | WV- | 13.55 | 18.53 | 16.04 | 28.86 | 33.37 | 31.11 | 48.85 | 55.87 | 52.36 |
| 46475 | 42588 | | | | | | | | | |
| WV- | WV- | 13.65 | 20.13 | 16.89 | 28.42 | 25.05 | 26.74 | 42.87 | 42.84 | 42.86 |
| 46476 | 42588 | | | | | | | | | |
| WV- | WV- | 13.89 | 23.53 | 18.71 | 40.03 | 32.41 | 36.22 | 54.62 | 57.70 | 56.16 |
| 46477 | 42588 | | | | | | | | | |
| WV- | WV- | 14.90 | 12.48 | 13.69 | 29.40 | 34.10 | 31.75 | 56.10 | 50.67 | 53.38 |
| 45022 | 42588 | | | | | | | | | |
| WV- | WV- | 18.71 | 13.60 | 16.16 | 49.35 | 45.25 | 47.30 | 57.69 | 59.74 | 58.71 |
| 42580 | 42588 | | | | | | | | | |
| WV- | WV- | 29.54 | 33.99 | 31.76 | 49.63 | 54.13 | 51.88 | 57.45 | 62.09 | 59.77 |
| 46478 | 42588 | | | | | | | | | |
| WV- | WV- | 23.69 | 13.52 | 18.61 | 46.44 | 40.30 | 43.37 | 69.84 | 58.17 | 64.00 |
| 46479 | 42588 | | | | | | | | | |
| WV- | WV- | 17.08 | 14.45 | 15.76 | 23.66 | 29.85 | 26.75 | 53.17 | 41.74 | 47.45 |
| 46480 | 42588 | | | | | | | | | |
| WV- | WV- | 26.18 | 28.37 | 27.28 | 52.42 | 40.53 | 46.47 | 69.15 | 60.57 | 64.86 |
| 46481 | 42588 | | | | | | | | | |
| WV- | WV- | 18.86 | 11.49 | 15.17 | 33.78 | 19.88 | 26.83 | 45.31 | 48.14 | 46.72 |
| 46482 | 42588 | | | | | | | | | |
| WV- | WV- | 8.65 | 16.15 | 12.40 | 20.17 | 18.84 | 19.50 | 64.34 | 57.16 | 60.75 |
| 46483 | 42588 | | | | | | | | | |
| WV- | WV- | 11.95 | 12.40 | 12.17 | 17.82 | 23.09 | 20.45 | 40.08 | 46.87 | 43.48 |
| 42383 | 42427 | | | | | | | | | |
| WV- | WV- | 15.09 | 9.01 | 12.05 | 23.89 | 21.72 | 22.81 | 43.74 | 73.66 | 58.70 |
| 45041 | 42589 | | | | | | | | | |
| WV- | WV- | 14.15 | 9.83 | 11.99 | 22.45 | 27.84 | 25.14 | 49.33 | 52.71 | 51.02 |
| 46484 | 42589 | | | | | | | | | |
| WV- | WV- | 13.18 | 13.17 | 13.17 | 49.66 | 34.27 | 41.97 | 51.41 | 50.97 | 51.19 |
| 46485 | 42589 | | | | | | | | | |
| WV- | WV- | 13.49 | 13.61 | 13.55 | 26.24 | 23.84 | 25.04 | 46.44 | 46.51 | 46.48 |
| 46486 | 42589 | | | | | | | | | |
| WV- | WV- | 8.31 | 3.32 | 5.81 | 10.13 | 17.07 | 13.60 | 32.37 | 27.06 | 29.71 |
| 46487 | 42589 | | | | | | | | | |
| WV- | WV- | 13.95 | 19.82 | 16.88 | 36.04 | 23.74 | 29.89 | 61.12 | 69.45 | 65.29 |
| 46488 | 42589 | | | | | | | | | |
| WV- | WV- | 12.03 | 6.32 | 9.18 | 18.00 | 13.49 | 15.75 | 37.30 | 42.45 | 39.88 |
| 46489 | 42589 | | | | | | | | | |
| WV- | WV- | 5.85 | 10.36 | 8.11 | 18.68 | 17.12 | 17.90 | 32.64 | 30.58 | 31.61 |
| 46490 | 42589 | | | | | | | | | |
| WV- | WV- | 3.64 | 3.68 | 3.66 | 14.94 | 7.02 | 10.98 | 31.75 | 33.72 | 32.73 |
| 45042 | 42589 | | | | | | | | | |
| WV- | WV- | 11.09 | 6.44 | 8.77 | 30.91 | 21.33 | 26.12 | 42.47 | 43.94 | 43.20 |
| 42581 | 42589 | | | | | | | | | |
| WV- | WV- | 11.22 | 14.39 | 12.80 | 29.64 | 29.84 | 29.74 | 59.70 | 55.35 | 57.52 |
| 46491 | 42589 | | | | | | | | | |
| WV- | WV- | 8.15 | 8.71 | 8.43 | 24.23 | 21.16 | 22.70 | 43.38 | 37.88 | 40.63 |
| 46492 | 42589 | | | | | | | | | |
| WV- | WV- | 3.63 | 6.21 | 4.92 | 8.27 | 6.38 | 7.33 | 34.50 | 37.73 | 36.12 |
| 46493 | 42589 | | | | | | | | | |
| WV- | WV- | 22.41 | 11.21 | 16.81 | 34.02 | 25.92 | 29.97 | 73.94 | 76.25 | 75.09 |
| 46494 | 42589 | | | | | | | | | |
| WV- | WV- | N.D. | 6.93 | 6.93 | 17.34 | 8.36 | 12.85 | 36.07 | 46.42 | 41.24 |
| 46495 | 42589 | | | | | | | | | |
| WV- | WV- | 7.55 | 4.59 | 6.07 | 10.91 | 9.80 | 10.35 | 32.16 | 30.93 | 31.54 |
| 46496 | 42589 | | | | | | | | | |

Table 14 shows IC50 of knocking down human HSD17B13 mRNA in human primary hepatocyte.

TABLE 14

| Guide | Passenger | IC50 (pM) | 95% CI |
|---|---|---|---|
| WV-43135 | WV-44173 | 44.94 | 17.77 to 105.8 |
| WV-42383 | WV-42427 | 92.7 | 54.70 to 159.0 |
| WV-45041 | WV-42589 | 22.61 | 10.14 to 49.82 |
| WV-46490 | WV-42589 | 20.85 | 12.62 to 34.38 |
| WV-47138 | WV-42589 | 17.74 | 7.51 to 40.83 |
| WV-46487 | WV-42589 | 42.27 | 20.98 to 83.38 |
| WV-47158 | WV-42589 | 39.69 | 23.27 to 67.98 |
| WV-45042 | WV-42589 | 26.68 | 14.09 to 50.38 |
| WV-46496 | WV-42589 | 24.9 | 12.74 to 48.46 |
| WV-47139 | WV-42589 | 19.43 | 11.97 to 31.42 |
| WV-46493 | WV-42589 | 18.95 | 10.53 to 34.62 |
| WV-47159 | WV-42589 | 20.27 | 8.63 to 48.06 |

Table 15 shows IC50 of knocking down human HSD17B13 mRNA in human primary hepatocyte.

TABLE 15

| Guide | Passenger | IC50 (pM) | 95% CI |
|---|---|---|---|
| WV-47139 | WV-42589 | 156.5 | 35.13 to 555.5 |
| WV-47159 | WV-42589 | 177.7 | 96.48 to 329.6 |
| WV-49590 | WV-42589 | 123.6 | 72.21 to 209.2 |
| WV-49591 | WV-42589 | 277.9 | 130.2 to 604.9 |
| WV-49592 | WV-42427 | 730.5 | 378.8 to 1460 |
| WV-49593 | WV-42427 | 752.5 | 392.1 to 1534 |
| WV-42383 | WV-42427 | 394.3 | 198.8 to 802.2 |

### Example 3. Provided Oligonucleotides and Compositions are Active In Vivo

Among other things, the present disclosure demonstrated that provided oligonucleotides and compositions are active in vivo. In the present Example, animal procedures were performed under IACUC guidelines. To evaluate the potency, human HSD17B13 transgenic mice were dosed at 3 mg/kg on Day 1 by subcutaneous administration. Animals were euthanized on Day 8 by CO₂ asphyxiation followed by thoracotomy. After cardiac perfusion with PBS, liver samples were harvested and flash-frozen in dry ice. Processed serum samples were kept at −70° C. Liver total RNA was extracted using SV96 Total RNA Isolation kit (Promega), after tissue lysis with TRIzol and bromochloropropane. cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For human HSD17B13 mRNA, the following qPCR assay was utilized: Forward, 5′-CGAAGGGATCCTTACCTCATC-3′; Reverse, 5′-CCAAGGCCTGAAGTCTGAT-3′; Probe, 5′ATTTGCCGCTGTTGGCTTTCACAG-3′. Mouse HPRT was used as normalizer (Forward 5′-CAAACTTTGCTTTCCCTGGT-3′, Reverse 5′-TGGCCTGTATCCAACACTTC-3′, Probe 5′-ACCAGCAAGCTTGCAACCTTAACC-3′.

Table 16 shows % human HSD17B13 mRNA remaining relative to PBS control. N=5. N.D.: Not determined.

TABLE 16

| | |
|---|---|
| | % remaining of |
| animal | hHSD17B13 |
| No. | mRNA |

PBS

| | |
|---|---|
| 1 | 101.01 |
| 2 | 101.43 |
| 3 | 97.68 |
| 4 | 105.62 |
| 5 | 94.26 |
| Mean | 100.00 |

WV-42576/
WV-42584

| | |
|---|---|
| 6 | 62.69 |
| 7 | 44.74 |
| 8 | 71.94 |
| 9 | 52.97 |
| 10 | 84.76 |
| Mean | 63.42 |

WV-42577/
WV-42585

| | |
|---|---|
| 11 | 56.84 |
| 12 | 65.95 |
| 13 | 39.00 |
| 14 | 38.78 |
| 15 | N.D. |
| Mean | 50.14 |

WV-42578/
WV-42586

| | |
|---|---|
| 16 | N.D. |
| 17 | 56.67 |
| 18 | 92.65 |
| 19 | 48.41 |
| 20 | 68.44 |
| Mean | 66.54 |

WV-42579/
WV-42587

| | |
|---|---|
| 21 | 64.96 |
| 22 | 38.58 |
| 23 | 23.39 |
| 24 | N.D. |
| 25 | 47.42 |
| Mean | 43.59 |

WV-42580/
WV-42588

| | |
|---|---|
| 26 | 67.46 |
| 27 | 45.72 |
| 28 | 85.64 |
| 29 | 63.32 |
| 30 | 36.61 |
| Mean | 59.75 |

WV-42581/
WV-42589

| | |
|---|---|
| 31 | 18.25 |
| 32 | 34.95 |
| 33 | 48.25 |
| 34 | 58.59 |
| 35 | 61.91 |
| Mean | 44.39 |

WV-42582/
WV-42590

| | |
|---|---|
| 36 | 62.23 |
| 37 | 46.62 |
| 38 | 37.79 |
| 39 | 68.76 |
| 40 | 68.00 |
| Mean | 56.68 |

### Example 4. Provided Oligonucleotides and Compositions are Active In Vivo

Among other things, the present disclosure demonstrated that provided oligonucleotides and compositions are active in vivo. In the present Example, animal procedures were performed under IACUC guidelines. To evaluate the potency, human HSD17B13 transgenic mice were dosed at 5 mg/kg on Day 1 by subcutaneous administration. Animals were euthanized on Day 8 by CO₂ asphyxiation followed by thoracotomy. After cardiac perfusion with PBS, liver samples were harvested and flash-frozen in dry ice. Processed serum samples were kept at −70° C. Liver total RNA was extracted using SV96 Total RNA Isolation kit (Promega), after tissue lysis with TRIzol and bromochloropropane. cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For human HSD17B13 mRNA, the following qPCR assay was utilized: Forward, 5′-CGAAGGGATCCTTACCTCATC-3′; Reverse, 5′-CCAAGGCCTGAAGTCTGAT-3′; Probe, 5′ATTTGCCGCTGTTGGCTTTCACAG-3′. Mouse HPRT was used as normalizer (Forward 5′-CAAACTTTGCTTTCCCTGGTT-3′, Reverse 5′-TGGCCTGTATCCAACACTTC-3′, Probe 5′-ACCAGCAAGCTTGCAACCTTAACC-3′.

Table 17 shows % human HSD17B13 mRNA remaining relative to PBS control. N=7. N.D.: Not determined.

TABLE 17

| | |
|---|---|
| | % remaining of |
| animal | hHSD17B13 |
| No. | mRNA |

PBS

| | |
|---|---|
| 1 | 107.09 |
| 2 | 100.15 |
| 3 | 88.98 |
| 4 | 101.64 |
| 5 | 101.25 |
| 6 | 100.89 |
| 7 | N.D. |
| Mean | 100.00 |

WV-42373/
WV-42417

| | |
|---|---|
| 8 | 32.64 |
| 9 | 39.40 |
| 10 | 25.35 |
| 11 | 31.78 |
| 12 | 36.63 |
| 13 | 36.04 |
| 14 | 35.28 |
| Mean | 33.88 |

WV-42381/
WV-42425

| | |
|---|---|
| 15 | 39.70 |
| 16 | 38.68 |
| 17 | 36.11 |
| 18 | 34.22 |
| 19 | 46.27 |
| 20 | 34.14 |
| 21 | 48.90 |
| Mean | 39.71 |

WV-42383/
WV-42427

| | |
|---|---|
| 22 | 28.74 |
| 23 | 34.78 |
| 24 | 33.39 |
| 25 | 32.06 |
| 26 | 32.34 |
| 27 | 39.08 |
| 28 | 53.22 |
| Mean | 36.23 |

WV-42392/
WV-42436

| | |
|---|---|
| 29 | 140.71 |
| 30 | 98.76 |
| 31 | 108.37 |
| 32 | 138.59 |
| 33 | 102.37 |
| 34 | 100.42 |
| 35 | 100.58 |
| Mean | 112.83 |

WV-43135/
WV-44173

| | |
|---|---|
| 36 | 34.59 |
| 37 | 60.53 |
| 38 | 63.31 |
| 39 | 45.67 |
| 40 | 71.15 |
| 41 | 47.75 |
| 42 | 64.02 |
| Mean | 55.29 |

WV-43136/
WV-44174

| | |
|---|---|
| 43 | 9.05 |
| 44 | 41.89 |
| 45 | 30.35 |
| 46 | 39.11 |
| 47 | 39.96 |
| 48 | 37.13 |
| 49 | 42.39 |
| Mean | 34.27 |

### Example 5. Provided Oligonucleotides and Compositions are Active In Vivo

Among other things, the present disclosure demonstrated that provided oligonucleotides and compositions are active in vivo. In the present Example, animal procedures were performed under IACUC guidelines. To evaluate the potency, human HSD171B13 transgenic mice were dosed at desired oligonucleotide concentration on Day 1 by subcutaneous administration. Animals were euthanized on Day 8 by CO₂ asphyxiation followed by thoracotomy. After cardiac perfusion with PBS, liver samples were harvested and flash-frozen in dry ice. Processed serum samples were kept at −70° C. Liver total RNA was extracted using SV96 Total RNA Isolation kit (Promega), after tissue lysis with TRIzol and bromochloropropane. cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For human HSD17B13 mRNA, the following qPCR assay was utilized: Forward, 5′-CGAAGGGATCCTTACCTCATC-3′; Reverse, 5′-CCAAGGCCTGAAGTCTGAT-3′; Probe, 5′ATTTGCCGCTGTTGGCTTTCACAG-3′. Mouse HPRT was used as normalizer (Forward 5′-CAAACTTTGCTTTCCCTGGT-3′, Reverse 5′-TGGCCTGTATCCAACACTTC-3′, Probe 5′-ACCAGCAAGCTTGCAACCTTAACC-3′.

Table 18 shows % human HSD7B13 mRNA remaining relative to PBS control. N=5. N.D.: Not determined.

TABLE 18

| | |
|---|---|
| | % remaining of |
| animal | hHSD17B13 |
| No. | mRNA |

PBS

| | |
|---|---|
| 1 | 98.63 |
| 2 | 104.66 |
| 3 | 108.25 |
| 4 | 82.46 |
| 5 | 106.01 |
| Mean | 100.00 |

WV-43135/
WV-44173
3 mg/kg

| | |
|---|---|
| 6 | 42.68 |
| 7 | 40.07 |
| 8 | 44.55 |
| 9 | 29.68 |
| 10 | 49.09 |
| Mean | 41.21 |

WV-42373/
WV-42417
1 mg/kg

| | |
|---|---|
| 11 | 54.55 |
| 12 | 54.14 |
| 13 | 64.07 |
| 14 | 61.94 |
| 15 | 58.42 |
| Mean | 58.63 |

WV-42373/
WV-42417
3 mg/kg

| | |
|---|---|
| 16 | 37.77 |
| 17 | 36.90 |
| 18 | 30.32 |
| 19 | 46.35 |
| 20 | 49.20 |
| Mean | 40.11 |

WV-42373/
WV-42417
10 mg/kg

| | |
|---|---|
| 21 | 10.94 |
| 22 | 33.04 |
| 23 | 8.59 |
| 24 | 23.00 |
| 25 | 29.15 |
| Mean | 20.94 |

WV-42383/
WV-42427
1 mg/kg

| | |
|---|---|
| 26 | 76.03 |
| 27 | 85.94 |
| 28 | 70.69 |
| 29 | 71.36 |
| 30 | 93.10 |
| Mean | 79.42 |

WV-42383/
WV-42427
3 mg/kg

| | |
|---|---|
| 31 | 33.38 |
| 32 | 19.14 |
| 33 | 45.10 |
| 34 | 47.61 |
| 35 | 30.32 |
| Mean | 35.11 |

WV-42383/
WV-42427
10 mg/kg

| | |
|---|---|
| 36 | 24.94 |
| 37 | 15.73 |
| 38 | 12.63 |
| 39 | 17.41 |
| 40 | 18.75 |
| Mean | 17.89 |

### Example 6. Provided Oligonucleotides and Compositions are Active In Vivo

Among other things, the present disclosure demonstrated that provided oligonucleotides and compositions are active in vivo. In the present Example, animal procedures were performed under IACUC guidelines. To evaluate the potency and liver exposure of provided oligonucleotides and compositions, human HSD17B13 transgenic mice were dosed at 3 mg/kg on Day 1 by subcutaneous administration. Animals were euthanized on Day 8 by CO₂ asphyxiation followed by thoracotomy. After cardiac perfusion with PBS, liver samples were harvested and flash-frozen in dry ice. Processed serum samples were kept at −70° C. Liver total RNA was extracted using SV96 Total RNA Isolation kit (Promega), after tissue lysis with TRIzol and bromochloropropane. cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For human HSD17B13 mRNA, the following qPCR assay was utilized: Forward, 5′-CGAAGGGATCCTTACCTCATC-3′; Reverse, 5′-CCAAGGCCTGAAGTTCTGAT-3′; Probe, 5′ATTTGCCGCTGTGGCTTTCACAG-3′.

Mouse HPRT was used as normalizer (Forward 5′-CAAACTTTGCTTTCCCTGGTT-3′, Reverse 5′-TGGCCTGTATCCAACACTC-3′, Probe 5′-ACCAGCAAGCTTGCAACCTrAACC-3′. Oligonucleotide accumulation in liver was determined by hybrid ELISA.

Table 19 shows % human HSD17B13 mRNA remaining relative to PBS control. N=5. N.D.: Not determined.

TABLE 19

| | |
|---|---|
| | % remaining of |
| animal | hHSD17B13 |
| No. | mRNA |

PBS

| | |
|---|---|
| 1 | 97.65 |
| 2 | 114.78 |
| 3 | 96.99 |
| 4 | 97.05 |
| 5 | 93.53 |
| Mean | 100.00 |

WV-43135/
WV-44173

| | |
|---|---|
| 6 | 15.05 |
| 7 | 62.14 |
| 8 | 20.88 |
| 9 | 49.44 |
| 10 | 9.49 |
| Mean | 31.40 |

WV-42383/
WV-42427

| | |
|---|---|
| 11 | 26.80 |
| 12 | 40.23 |
| 13 | 40.75 |
| 14 | 53.79 |
| 15 | 38.76 |
| Mean | 40.07 |

WV-46490/
WV-42589

| | |
|---|---|
| 16 | 20.93 |
| 17 | 33.02 |
| 18 | 22.44 |
| 19 | 87.91 |
| 20 | 16.95 |
| Mean | 36.25 |

WV-47138/
WV-42589

| | |
|---|---|
| 21 | 33.11 |
| 22 | 25.87 |
| 23 | 22.73 |
| 24 | 17.60 |
| 25 | 20.26 |
| Mean | 23.91 |

WV-47158/
WV-42589

| | |
|---|---|
| 26 | 20.58 |
| 27 | 58.00 |
| 28 | 44.67 |
| 29 | 41.26 |
| 30 | 28.58 |
| Mean | 38.62 |

WV-46496/
WV-42589

| | |
|---|---|
| 31 | 15.24 |
| 32 | 16.12 |
| 33 | 12.72 |
| 34 | 13.37 |
| 35 | 22.60 |
| Mean | 16.01 |

WV-47139/
WV-42589

| | |
|---|---|
| 36 | 18.83 |
| 37 | 31.92 |
| 38 | 18.38 |
| 39 | 16.70 |
| 40 | 27.95 |
| Mean | 22.76 |

WV-47159/
WV-42589

| | |
|---|---|
| 41 | 24.78 |
| 42 | 20.26 |
| 43 | N.D. |
| 44 | 18.15 |
| 45 | 18.26 |
| Mean | 20.37 |

Table 20 shows % accumulation of antisense strand in liver tissue. N=5. N.D.: Not determined.

TABLE 20

| | |
|---|---|
| | antisense |
| animal | strand (μg/g |
| No. | of tissue) |

PBS

| | |
|---|---|
| 1 | 0.00 |
| 2 | 0.00 |
| 3 | 0.00 |
| 4 | 0.07 |
| 5 | 0.00 |
| Mean | 0.01 |

WV-42383/
WV-42427

| | |
|---|---|
| 11 | 6.41 |
| 12 | 7.18 |
| 13 | 5.91 |
| 14 | 8.01 |
| 15 | 4.96 |
| Mean | 6.49 |

WV-46490/
WV-42589

| | |
|---|---|
| 16 | 6.82 |
| 17 | 8.95 |
| 18 | 9.08 |
| 19 | N.D. |
| 20 | 6.87 |
| Mean | 7.93 |

WV-47138/
WV-42589

| | |
|---|---|
| 21 | 8.09 |
| 22 | 8.64 |
| 23 | 11.11 |
| 24 | 11.80 |
| 25 | 13.08 |
| Mean | 10.54 |

WV-47158/
WV-42589

| | |
|---|---|
| 26 | 8.51 |
| 27 | 8.43 |
| 28 | 10.14 |
| 29 | 11.00 |
| 30 | 6.21 |
| Mean | 8.86 |

WV-46496/
WV-42589

| | |
|---|---|
| 31 | 7.35 |
| 32 | 9.14 |
| 33 | 7.10 |
| 34 | 7.06 |
| 35 | 8.86 |
| Mean | 7.90 |

WV-47139/
WV-42589

| | |
|---|---|
| 36 | 10.27 |
| 37 | 9.81 |
| 38 | 8.91 |
| 39 | 9.83 |
| 40 | 6.87 |
| Mean | 9.14 |

WV-47159/
WV-42589

| | |
|---|---|
| 41 | 7.61 |
| 42 | 6.71 |
| 43 | 6.95 |
| 44 | 7.77 |
| 45 | 8.02 |
| Mean | 7.41 |

### Example 7. Provided Oligonucleotides and Compositions are Active In Vivo

Among other things, the present disclosure demonstrated that provided oligonucleotides and compositions are active in vivo. In the present Example, animal procedures were performed under IACUC guidelines. To evaluate the potency and liver exposure of provided oligonucleotides and compositions, human HSD17B13 transgenic mice were dosed at 3 mg/kg on Day 1 by subcutaneous administration. Animals were euthanized on Day 8, 22, and 48, by CO₂ asphyxiation followed by thoracotomy. After cardiac perfusion with PBS, liver samples were harvested and flash-frozen in dry ice. Processed serum samples were kept at −70° C. Liver total RNA was extracted using SV96 Total RNA Isolation kit (Promega), after tissue lysis with TRIzol and bromochloropropane. cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For human HSD17B13 mRNA, the following qPCR assay was utilized: Forward, 5′-CGAAGGGATCCTTACCTCATC-3′; Reverse, 5′-CCAAGGCCTGAAGTTCTGAT-3′; Probe, 5′ATTTGCCGCTGTTGGCTTTCACAG-3′. Mouse HPRT was used as normalizer (Forward 5′-CAAACTTTGCTTTCCCTGGTT-3′, Reverse 5′-TGGCCTGTATCCAACACTC-3′, Probe 5′-ACCAGCAAGCTTGCAACCTrAACC-3′. Oligonucleotide accumulation in liver was determined by hybrid ELISA.

Ago2 immunoprecipitation assay: Tissues (1 mpk dosed) were lysed in lysis buffer 50 mM Tris-HCl at pH 7.5, 200 mM NaCl, 0.5% Triton X-100, 2 mM EDTA, 1 mg/mL heparin) with protease inhibitor (Sigma-Aldrich). Lysate concentration was measured with a protein BCA kit (Pierce BCA protein assay kit or Bradford protein assay kit). Anti-Ago2 antibody was purchased from Wako Chemicals. Control mouse IgG was from eBioscience. Dynabeads (Invitrogen) were used to precipitate antibodies. Ago2-associated siRNA and endogenous miR122 were measured by Stem-Loop RT followed by TaqMan PCR analysis using Taqman mniRNA and siRNA assay kit (Thermo fisher) based on manufacturer's methods.

Table 21 shows % human HSD17B13 mRNA remaining relative to PBS control. N=4 or 5. N.D.: Not determined.

TABLE 21

| | |
|---|---|
| | % remaining of |
| animal | hHSD17B13 |
| No. | mRNA |

Day 8
PBS

| | |
|---|---|
| 1 | 106.61 |
| 2 | 118.82 |
| 3 | 74.58 |
| 4 | N.D. |
| Mean | 100.00 |

Day 8
WV-42383/
WV-42427

| | |
|---|---|
| 5 | 35.70 |
| 6 | 48.27 |
| 7 | 57.48 |
| 8 | 54.22 |
| 9 | 64.55 |
| Mean | 52.04 |

Day 8
WV-47139/
WV-42589

| | |
|---|---|
| 10 | 28.50 |
| 11 | 17.15 |
| 12 | 28.92 |
| 13 | 21.43 |
| 14 | 34.20 |
| Mean | 26.04 |

Day 22
PBS

| | |
|---|---|
| 15 | 110.98 |
| 16 | N.D. |
| 17 | 104.61 |
| 18 | 84.41 |
| Mean | 100.00 |

Day 22
WV-42383/
WV-42427

| | |
|---|---|
| 19 | 20.92 |
| 20 | 24.13 |
| 21 | 38.59 |
| 22 | 45.13 |
| 23 | 65.63 |
| Mean | 38.88 |

Day 22
WV-47139/
WV-42589

| | |
|---|---|
| 24 | 16.05 |
| 25 | 6.63 |
| 26 | 10.31 |
| 27 | 17.81 |
| 28 | 33.19 |
| Mean | 16.80 |

Day 48
PBS

| | |
|---|---|
| 29 | 124.59 |
| 30 | 108.81 |
| 31 | 90.53 |
| 32 | 76.07 |
| Mean | 100.00 |

Day 48
WV-42383/
WV-42427

| | |
|---|---|
| 33 | 46.63 |
| 34 | 60.18 |
| 35 | 47.92 |
| 36 | 56.26 |
| 37 | 101.66 |
| Mean | 62.53 |

Day 48
WV-47139/
WV-42589

| | |
|---|---|
| 38 | 38.44 |
| 39 | 29.55 |
| 40 | 29.89 |
| 41 | 24.51 |
| 42 | N.D. |
| Mean | 30.60 |

Table 22 shows % accumulation of antisense strand in liver tissue. N=4 or 5. N.D.: Not determined.

TABLE 22

| | |
|---|---|
| | antisense |
| animal | strand (μg/g |
| No. | of tissue) |

Day 8
PBS

| | |
|---|---|
| 1 | 0.00 |
| 2 | 0.00 |
| 3 | 0.00 |
| 4 | 0.00 |
| Mean | 0.00 |

Day 8
WV-42383/
WV-42427

| | |
|---|---|
| 5 | 5.97 |
| 6 | 6.53 |
| 7 | 6.63 |
| 8 | 4.81 |
| 9 | 6.01 |
| Mean | 5.99 |

Day 8
WV-47139/
WV-42589

| | |
|---|---|
| 10 | 8.69 |
| 11 | 9.92 |
| 12 | 9.83 |
| 13 | 9.23 |
| 14 | 9.07 |
| Mean | 9.35 |

Day 22
PBS

| | |
|---|---|
| 15 | 0.00 |
| 16 | 0.00 |
| 17 | 0.00 |
| 18 | 0.00 |
| Mean | 0.00 |

Day 22
WV-42383/
WV-42427

| | |
|---|---|
| 19 | 2.93 |
| 20 | 2.70 |
| 21 | 2.73 |
| 22 | 2.77 |
| 23 | 3.05 |
| Mean | 2.84 |

Day 22
WV-47139/
WV-42589

| | |
|---|---|
| 24 | 3.87 |
| 25 | 4.37 |
| 26 | 4.41 |
| 27 | 4.63 |
| 28 | 4.69 |
| Mean | 4.39 |

Day 48
PBS

| | |
|---|---|
| 29 | 0.00 |
| 30 | 0.00 |
| 31 | 0.00 |
| 32 | 0.00 |
| Mean | 0.00 |

Day 48
WV-42383/
WV-42427

| | |
|---|---|
| 33 | 1.20 |
| 34 | 1.02 |
| 35 | 0.67 |
| 36 | 0.73 |
| 37 | 0.61 |
| Mean | 0.85 |

Day 48
WV-47139/
WV-42589

| | |
|---|---|
| 38 | 1.89 |
| 39 | 1.68 |
| 40 | 1.83 |
| 41 | 1.72 |
| 42 | 1.82 |
| Mean | 1.79 |

Table 23 shows % Ago2 loading of antisense strand relative to miR-122. N=3.

TABLE 23

| | | | | | Relative |
|---|---|---|---|---|---|
| | Ct: | Ct: miR- | Ct: | Ct: miR- | HSD/miR- |
| | HSD/Ago2 | 122/Ago2 | HSD/IgG | 122/IgG | 122/WV-42383 |
| WV-42383 | 27.5 | 17.2 | 37.6 | 27.5 | 0.97 |
| day 8 # 5-1 | | | | | |
| WV-42383 | 28.3 | 17.1 | 37.2 | 26.8 | 0.56 |
| day 8 # 6-1 | | | | | |
| WV-42383 | 26.9 | 17.5 | 36.0 | 27.2 | 1.84 |
| day 8 # 7-1 | | | | | |
| WV-47139 | 25.3 | 17.8 | 34.0 | 28.1 | 7.18 |
| day 8 # 10-1 | | | | | |
| WV-47139 | 24.9 | 17.7 | 34.1 | 28.4 | 8.44 |
| day 8 # 11-1 | | | | | |
| WV-47139 | 25.9 | 18.0 | 34.7 | 28.2 | 5.37 |
| day 8 # 12-1 | | | | | |
| WV-42383 | 28.7 | 17.5 | 38.3 | 28.3 | 0.53 |
| day 22 # 19-1 | | | | | |
| WV-42383 | 29.3 | 17.7 | 38.8 | 28.4 | 0.43 |
| day 22 # 20-1 | | | | | |
| WV-42383 | 27.3 | 17.0 | 37.4 | 28.3 | 1.03 |
| day 22 # 21-1 | | | | | |
| WV-47139 | 25.4 | 17.7 | 34.3 | 27.7 | 6.27 |
| day 22 # 24-1 | | | | | |
| WV-47139 | 26.5 | 18.3 | 37.0 | 29.8 | 4.34 |
| day 22 # 25-1 | | | | | |
| WV-47139 | 29.7 | 19.9 | 38.2 | 29.8 | 1.43 |
| day 22 # 26-1 | | | | | |
| WV-42383 | 30.4 | 16.9 | 39.1 | 27.1 | 0.11 |
| day 48 # 33-1 | | | | | |
| WV-42383 | 31.2 | 17.1 | 40.0 | 27.2 | 0.07 |
| day 48 # 34-1 | | | | | |
| WV-42383 | 29.9 | 17.0 | 40.0 | 28.4 | 0.17 |
| day 48 # 35-1 | | | | | |
| WV-47139 | 28.6 | 17.2 | 39.7 | 28.6 | 0.49 |
| day 48 # 38-1 | | | | | |
| WV-47139 | 26.5 | 16.9 | 38.2 | 28.8 | 1.63 |
| day 48 # 39-1 | | | | | |
| WV-47139 | 28.9 | 17.6 | 40.0 | 28.6 | 0.48 |
| day 48 # 40-1 | | | | | |

### Example 8. Provided Oligonucleotides and Compositions are Active In Vivo

Among other things, the present disclosure demonstrated that provided oligonucleotides and compositions are active in vivo. In the present Example, animal procedures were performed under IACUC guidelines. To evaluate the potency and liver exposure of provided oligonucleotides and compositions, human HSD17B13 transgenic mice were dosed at 3 mg/kg on Day 1 by subcutaneous administration. Animals were euthanized on Day 15, 50, and 99, by CO₂ asphyxiation followed by thoracotomy. After cardiac perfusion with PBS, liver samples were harvested and flash-frozen in dry ice. Processed serum samples were kept at −70° C. Liver total RNA was extracted using SV96 Total RNA Isolation kit (Promega), after tissue lysis with TRIzol and bromochloropropane. cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For human HSD17B13 mRNA, the following qPCR assay was utilized: Forward, 5′-CGAAGGGATCCTTACCTCATC-3′; Reverse, 5′-CCAAGGCCTGAAGTTCTGAT-3′; Probe, 5′ATTTGCCGCTGTTGGCTTTCACAG-3′. Mouse HPRT was used as normalizer (Forward 5′-CAAACTTTGCTTTCCCTGGTT-3′, Reverse 5′-TGGCCTGTATCCAACACTC-3′, Probe 5′-ACCAGCAAGCTTGCAACCTrAACC-3′. Oligonucleotide accumulation in liver was determined by hybrid ELISA.

Ago2 immunoprecipitation assay: Tissues (1 mpk dosed) were lysed in lysis buffer 50 mM Tris-HCl at pH 7.5, 200 mM NaCl, 0.5% Triton X-100, 2 mM EDTA, 1 mg/mL heparin) with protease inhibitor (Sigma-Aldrich). Lysate concentration was measured with a protein BCA kit (Pierce BCA protein assay kit or Bradford protein assay kit). Anti-Ago2 antibody was purchased from Wako Chemicals. Control mouse IgG was from eBioscience. Dynabeads (Invitrogen) were used to precipitate antibodies. Ago2-associated siRNA and endogenous miR122 were measured by Stem-Loop RT followed by TaqMan PCR analysis using Taqman miRNA and siRNA assay kit (Thermo fisher) based on manufacturer's methods.

Table 24 shows % human HSD17B13 mRNA remaining relative to PBS control. N=4 or 5. N.D.: Not determined.

TABLE 24

| | |
|---|---|
| | % remaining of |
| animal | hHSD17B13 |
| No. | mRNA |

Day 15
PBS

| | |
|---|---|
| 1 | 93.16 |
| 2 | 106.84 |
| 3 | 108.47 |
| 4 | 91.53 |
| Mean | 100.00 |

Day 15
WV-49593/
WV-42427

| | |
|---|---|
| 5 | 86.34 |
| 6 | 49.78 |
| 7 | 46.33 |
| 8 | 39.66 |
| Mean | 55.53 |

Day 15
WV-49592/
WV-42427

| | |
|---|---|
| 9 | 47.68 |
| 10 | 83.29 |
| 11 | 34.36 |
| 12 | 50.27 |
| 13 | 33.25 |
| Mean | 49.77 |

Day 15
WV-49591/
WV-42589

| | |
|---|---|
| 14 | 20.41 |
| 15 | 32.98 |
| 16 | 15.58 |
| 17 | 15.39 |
| 18 | 18.81 |
| Mean | 20.64 |

Day 15
WV-49590/
WV-42589

| | |
|---|---|
| 19 | 17.78 |
| 20 | 26.90 |
| 21 | 21.57 |
| 22 | 26.86 |
| 23 | 34.37 |
| Mean | 25.50 |

Day 50
PBS

| | |
|---|---|
| 24 | 80.16 |
| 25 | 111.46 |
| 26 | 108.37 |
| 27 | N.D. |
| Mean | 100.00 |

Day 50
WV-49593/
WV-42427

| | |
|---|---|
| 28 | 41.18 |
| 29 | 40.40 |
| 30 | 38.11 |
| 31 | 29.13 |
| Mean | 37.21 |

Day 50
WV-49592/
WV-42427

| | |
|---|---|
| 32 | 38.41 |
| 33 | 68.24 |
| 34 | 70.70 |
| 35 | 47.64 |
| 36 | 52.67 |
| Mean | 55.53 |

Day 50
WV-49591/
WV-42589

| | |
|---|---|
| 37 | 44.44 |
| 38 | 26.00 |
| 39 | 32.62 |
| 40 | 29.50 |
| 41 | 44.12 |
| Mean | 35.34 |

Day 50
WV-49590/
WV-42589

| | |
|---|---|
| 42 | 16.62 |
| 43 | 18.79 |
| 44 | 23.48 |
| 45 | 20.72 |
| 46 | 18.62 |
| Mean | 19.65 |

Day 99
PBS

| | |
|---|---|
| 47 | 115.07 |
| 48 | 71.95 |
| 49 | 106.52 |
| 50 | 106.47 |
| Mean | 100.00 |

Day 99
WV-49593/
WV-42427

| | |
|---|---|
| 51 | 80.60 |
| 52 | 99.87 |
| 53 | 84.43 |
| 54 | 120.85 |
| Mean | 96.44 |

Day 99
WV-49592/
WV-42427

| | |
|---|---|
| 55 | 97.46 |
| 56 | 105.91 |
| 57 | 79.85 |
| 58 | 73.97 |
| 59 | 87.64 |
| Mean | 88.97 |

Day 99
WV-49591/
WV-42589

| | |
|---|---|
| 60 | 54.02 |
| 61 | 48.91 |
| 62 | 29.16 |
| 63 | 40.87 |
| 64 | 64.15 |
| Mean | 47.42 |

Day 99
WV-49590/
WV-42589

| | |
|---|---|
| 65 | 27.70 |
| 66 | 17.53 |
| 67 | 20.69 |
| 68 | 20.61 |
| 69 | 16.64 |
| Mean | 20.64 |

Table 25 shows % accumulation of antisense strand in liver tissue. N=4 or 5. N.D.: Not determined.

TABLE 25

| | |
|---|---|
| | antisense |
| animal | strand (μg/g |
| No. | of tissue) |

Day 15
PBS

| | |
|---|---|
| 1 | 0.00 |
| 2 | 0.00 |
| 3 | 0.00 |
| 4 | 0.00 |
| Mean | 0.00 |

Day 15
WV-49593/
WV-42427

| | |
|---|---|
| 5 | 2.35 |
| 6 | 2.54 |
| 7 | 2.68 |
| 8 | 2.96 |
| Mean | 2.63 |

Day 15
WV-49592/
WV-42427

| | |
|---|---|
| 9 | 2.56 |
| 10 | 2.65 |
| 11 | 2.56 |
| 12 | 2.55 |
| 13 | 2.19 |
| Mean | 2.50 |

Day 15
WV-49591/
WV-42589

| | |
|---|---|
| 14 | 3.08 |
| 15 | 3.19 |
| 16 | 2.91 |
| 17 | 2.59 |
| 18 | 3.13 |
| Mean | 2.98 |

Day 15
WV-49590/
WV-42589

| | |
|---|---|
| 19 | 3.28 |
| 20 | 3.23 |
| 21 | 3.07 |
| 22 | 2.97 |
| 23 | 1.60 |
| Mean | 2.83 |

Day 50
PBS

| | |
|---|---|
| 24 | 0.00 |
| 25 | 0.00 |
| 26 | 0.00 |
| 27 | 0.00 |
| Mean | 0.00 |

Day 50
WV-49593/
WV-42427

| | |
|---|---|
| 28 | 0.48 |
| 29 | 0.41 |
| 30 | 0.49 |
| 31 | 0.60 |
| Mean | 0.49 |

Day 50
WV-49592/
WV-42427

| | |
|---|---|
| 32 | 0.54 |
| 33 | 0.54 |
| 34 | 0.51 |
| 35 | 0.55 |
| 36 | 0.49 |
| Mean | 0.53 |

Day 50
WV-49591/
WV-42589

| | |
|---|---|
| 37 | 0.61 |
| 38 | 0.54 |
| 39 | 0.68 |
| 40 | 0.55 |
| 41 | 0.59 |
| Mean | 0.59 |

Day 50
WV-49590/
WV-42589

| | |
|---|---|
| 42 | 0.56 |
| 43 | 0.62 |
| 44 | 0.65 |
| 45 | 0.61 |
| 46 | 0.65 |
| Mean | 0.62 |

Day 99
PBS

| | |
|---|---|
| 47 | 0.00 |
| 48 | 0.00 |
| 49 | 0.00 |
| 50 | 0.00 |
| Mean | 0.00 |

Day 99
WV-49593/
WV-42427

| | |
|---|---|
| 51 | 0.01 |
| 52 | 0.05 |
| 53 | 0.03 |
| 54 | 0.03 |
| Mean | 0.03 |

Day 99
WV-49592/
WV-42427

| | |
|---|---|
| 55 | 0.06 |
| 56 | 0.09 |
| 57 | 0.05 |
| 58 | 0.07 |
| 59 | 0.07 |
| Mean | 0.07 |

Day 99
WV-49591/
WV-42589

| | |
|---|---|
| 60 | 0.11 |
| 61 | 0.10 |
| 62 | 0.08 |
| 63 | 0.09 |
| 64 | 0.06 |
| Mean | 0.09 |

Day 99
WV-49590/
WV-42589

| | |
|---|---|
| 65 | 0.06 |
| 66 | 0.06 |
| 67 | 0.08 |
| 68 | 0.10 |
| 69 | 0.10 |
| Mean | 0.08 |

Table 26 shows % Ago2 loading of antisense strand relative to miR-122. N=2.

TABLE 26

| | | | | | Relative HSD/miR- |
|---|---|---|---|---|---|
| | Ct: | Ct: miR- | Ct: | Ct: miR- | 122 normalized with |
| | HSD/Ago2 | 122/Ago2 | HSD/IgG | 122/IgG | WV-49593 Day 15 |
| WV-49593-1 | 27.04 | 21.41 | 33.52 | 28.37 | 1.00 |
| Day 15 | | | | | |
| WV-49593-2 | 27.46 | 21.81 | 36.72 | 28.76 | 0.99 |
| Day 15 | | | | | |
| WV-49592-1 | 27 | 19.85 | 37.89 | 29.46 | 0.35 |
| Day 15 | | | | | |
| WV-49592-2 | 26.88 | 19.66 | 37.83 | 29.16 | 0.33 |
| Day 15 | | | | | |
| WV-49591-1 | 22.43 | 19.65 | 33.25 | 29.2 | 7.26 |
| Day 15 | | | | | |
| WV-49591-2 | 22.42 | 19.65 | 32.76 | 28.81 | 7.31 |
| Day 15 | | | | | |
| WV-49590-1 | 22.37 | 19.92 | 32.42 | 29.12 | 9.12 |
| Day 15 | | | | | |
| WV-49590-2 | 22.29 | 19.97 | 32.83 | 29.22 | 9.98 |
| Day 15 | | | | | |
| WV-49593-1 | 24.3 | 18.24 | 36.91 | 28.17 | 0.75 |
| Day 50 | | | | | |
| WV-49593-2 | 27.37 | 18.13 | 38.12 | 28.53 | 0.08 |
| Day 50 | | | | | |
| WV-49592-1 | 26.39 | 17.54 | 37.62 | 28.07 | 0.11 |
| Day 50 | | | | | |
| WV-49592-2 | 26.2 | 17.85 | 37.84 | 28.11 | 0.15 |
| Day 50 | | | | | |
| WV-49591-1 | 22.28 | 17.62 | 33.71 | 27.86 | 1.97 |
| Day 50 | | | | | |
| WV-49591-2 | 23.55 | 18.32 | 37.09 | 28.91 | 1.33 |
| Day 50 | | | | | |
| WV-49590-1 | 23.88 | 18.88 | 37.05 | 29.31 | 1.56 |
| Day 50 | | | | | |
| WV-49590-2 | 25.1 | 18.87 | 38.1 | 30.17 | 0.66 |
| Day 50 | | | | | |
| WV-49593-1 | 36.53 | 18.8 | 40 | 28.94 | 0.00 |
| Day 99 | | | | | |
| WV-49593-2 | 33.47 | 18.71 | 39.9 | 29.21 | 0.00 |
| Day 99 | | | | | |
| WV-49592-1 | 35.05 | 18.87 | 39.82 | 29.47 | 0.00 |
| Day 99 | | | | | |
| WV-49592-2 | 32.4 | 18.53 | 39.74 | 29.46 | 0.00 |
| Day 99 | | | | | |
| WV-49591-1 | 28.9 | 18.69 | 39.21 | 29.03 | 0.04 |
| Day 99 | | | | | |
| WV-49591-2 | 30.46 | 18.67 | 38.61 | 29.19 | 0.01 |
| Day 99 | | | | | |
| WV-49590-1 | 28.5 | 19.25 | 39.36 | 29.36 | 0.08 |
| Day 99 | | | | | |
| WV-49590-2 | 29.49 | 19.47 | 39.19 | 29.18 | 0.05 |
| Day 99 | | | | | |

### Example 9. Provided Oligonucleotides and Compositions can Effectively Knockdown HSD17B13 In Vitro

Various Double stranded oligonucleotides targeting HSD17B13 and compositions were designed, constructed, characterized and assessed. As appreciated by those skilled in the art, various technologies can be utilized to assess properties and/or activities of provided oligonucleotides and compositions thereof. Some of such technologies are described in this Example. Those skilled in the art appreciate that many other technologies can be readily utilized in accordance with the present disclosure. As demonstrated herein, double stranded oligonucleotides targeting HSD17B13 and compositions, among other things, can be highly active, e.g., in reducing levels of their target nucleic acids and proteins encoded thereby. A number of siRNAs were tested in vitro in NHP primary hepatocytes at a range of concentrations. Example protocol for in vitro determination of siRNA activity: For determination of siRNAs activity, siRNAs at specific concentration were gymotically delivered to NHP primary hepatocytes plated at 96-well plates, with 40,000 cells/well. Following 48 hours treatment, total RNA was extracted using SV96 Total RNA Isolation kit (Promega). cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For NHP HSD17B13, the following qPCR assay was utilized: Thermo Fisher Mf02888851-ml. SFRS9 was used as normalizer (Forward, 5′-TGGAATATGCCCTGCGTAAA-3′; Reverse, 5′-TGGTGCTTCTCTCAGGATAAAC-3′, Probe, 5′-TGGATGACACCAAATTCCGCTCTCA-3′. mRNA knockdown levels were calculated as % mRNA remaining relative to mock treatment.

Table 27 shows IC50 of knocking down NHP HSD17B13 mRNA in NHP primary hepatocyte.

TABLE 27

| Guide | Passenger | IC50 (pM) | 95% CI |
|---|---|---|---|
| WV-47139 | WV-42589 | 445.7 | 268.4 to 742.6 |
| WV-47159 | WV-42589 | 675.5 | 374.7 to 1217.0 |
| WV-49590 | WV-42589 | 1035.0 | 591.9 to 1840.0 |
| WV-49592 | WV-42427 | 2038.0 | 1091.0 to 3967.0 |
| WV-49593 | WV-42427 | 1378.0 | 730.9 to 2689.0 |
| WV-42383 | WV-42427 | 404.8 | 253.6 to 646.9 |

### Example 10. Provided Oligonucleotides and Compositions can Effectively Knockdown HSD17B13 In Vitro

Various Double stranded oligonucleotides targeting HSD17B13 and compositions were designed, constructed, characterized and assessed. As appreciated by those skilled in the art, various technologies can be utilized to assess properties and/or activities of provided oligonucleotides and compositions thereof. Some of such technologies are described in this Example. Those skilled in the art appreciate that many other technologies can be readily utilized in accordance with the present disclosure. As demonstrated herein, double stranded oligonucleotides targeting HSD17B13 and compositions, among other things, can be highly active, e.g., in reducing levels of their target nucleic acids and proteins encoded thereby.

A number of siRNAs were tested in vitro in human primary hepatocytes at a range of concentrations. Example protocol for in vitro determination of siRNA activity: For determination of siRNAs activity, siRNAs at specific concentration were gymotically delivered to human primary hepatocytes plated at 96-well plates, with 10,000 cells/well. Following 48 hours treatment, total RNA was extracted using SV96 Total RNA Isolation kit (Promega). cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For human HSD17B13 mRNA, the following qPCR assay was utilized: Forward, 5′-CGAAGGGATTCCTTACCTCATC-3′; Reverse, 5′-CCAAGGCCTGAAGTCTGAT-3′; Probe, 5′ATTTGCCGCTGTGGCTTTCACAG-3′.SFRS9 was used as normalizer (Forward, 5′-TGGAATATGCCCTGCGTAAA-3′; Reverse, 5′-TGGTGCTCTCTCAGGATAAAC-3′, Probe, 5′-TGGATGACACCAAATTCCGCTCTCA-3′. mRNA knockdown levels were calculated as % mRNA remaining relative to mock treatment.

Table 28 shows IC50 of knocking down human HSD17B13 mRNA in human primary hepatocyte.

TABLE 28

| Guide | Passenger | IC50 (pM) | 95% CI |
|---|---|---|---|
| WV-42383 | WV-42427 | 828.6 | 586.2 to 1176 |
| WV-49593 | WV-42427 | 759.5 | 431.6 to 1341 |
| WV-49592 | WV-42427 | 1836.0 | 1086 to 3140 |
| WV-47159 | WV-42589 | 363.3 | 247.6 to 536.6 |
| WV-49590 | WV-42589 | 557.8 | 370.1 to 843.2 |
| WV-49591 | WV-42589 | 505.1 | 347.6 to 734.5 |

### Example 11. Provided Oligonucleotides and Compositions are Active In Vivo

Among other things, the present disclosure demonstrated that provided oligonucleotides and compositions are active in vivo. In the present Example, animal procedures were performed under IACUC guidelines. To evaluate the potency and liver exposure of provided oligonucleotides and compositions, human HSD17B13 transgenic mice were dosed at 3 or 1.5 mg/kg on Day 1 by subcutaneous administration. Animals were euthanized on Day 90, by CO₂ asphyxiation followed by thoracotomy. After cardiac perfusion with PBS, liver samples were harvested and flash-frozen in dry ice. Liver total RNA was extracted using SV96 Total RNA Isolation kit (Promega), after tissue lysis with TRIzol and bromochloropropane. cDNA production from RNA samples were performed using High-Capacity cDNA Reverse Transcription kit (Thermo Fisher) following manufacturer's instructions and qPCR analysis performed in CFX System using iQ Multiplex Powermix (Bio-Rad). For human HSD17B13 mRNA, the following % PCR assay was utilized: Forward, 5′-CGAAGGGATCCTTACCTCATC-3′; Reverse, 5′-CCAAGGCCTGAAGTCTGAT-3′; Probe, 5′ATGCCGCTGTGGCTTCACAG-3′. Mouse HPRT was used as normalizer (Forward 5′-CAAACTTTGCTTTCCCTGGT-3′, Reverse 5′-TGGCCTGTATCCAACACTTC-3′, Probe 5′-ACCAGCAAGCTGCAACCTAACC-3′. Oligonucleotide accumulation in liver was determined by hybrid ELISA.

Table 29 shows % human HSD17B13 mRNA remaining relative to PBS control. N=4 or 5. N.D.: Not determined.

TABLE 29

| | |
|---|---|
| | % remaining of |
| animal | hHSD17B13 |
| No. | mRNA |

Day 90
PBS

| | |
|---|---|
| 1 | 90.50 |
| 2 | 111.09 |
| 3 | 96.42 |
| 4 | 101.99 |
| Mean | 100.00 |

Day 90
WV-49614/
WV-49615 (3 mpk)
(Non-targeting
Control)

| | |
|---|---|
| 5 | 61.99 |
| 6 | 95.13 |
| 7 | 78.86 |
| 8 | 79.34 |
| Mean | 78.83 |

Day 90
WV-42383/
WV-42427 (3 mpk)

| | |
|---|---|
| 9 | 101.24 |
| 10 | 37.67 |
| 11 | 56.06 |
| 12 | 38.85 |
| Mean | 58.46 |

Day 90
WV-49592/
WV-42427 (3 mpk)

| | |
|---|---|
| 13 | 31.88 |
| 14 | 47.88 |
| 15 | 57.80 |
| 16 | 27.74 |
| Mean | 41.33 |

Day 90
WV-49590/
WV-42589 (3 mpk)

| | |
|---|---|
| 17 | 27.13 |
| 18 | 33.22 |
| 19 | 27.93 |
| 20 | 31.11 |
| 21 | 19.98 |
| Mean | 27.88 |

Day 90
WV-49590/
WV-42589 (1.5 mpk)

| | |
|---|---|
| 22 | 23.35 |
| 23 | 24.26 |
| 24 | 31.20 |
| 25 | 29.10 |
| Mean | 26.98 |

Table 30 shows % accumulation of antisense strand in liver tissue. N=4 or 5. N.D.: Not determined Table 30

TABLE 30

| | |
|---|---|
| | antisense |
| animal | strand (μg/g |
| No. | of tissue) |

Day 90
PBS

| | |
|---|---|
| 1 | 0.00 |
| 2 | 0.00 |
| 3 | 0.00 |
| 4 | 0.00 |
| Mean | 0.00 |

Day 90
WV-49614/
WV-49615 (3 mpk)
(Non-targeting
Control)

| | |
|---|---|
| 5 | 0.03 |
| 6 | 0.04 |
| 7 | 0.03 |
| 8 | 0.04 |
| Mean | 0.04 |

Day 90
WV-42383/
WV-42427 (3 mpk)

| | |
|---|---|
| 9 | 0.06 |
| 10 | 0.15 |
| 11 | 0.04 |
| 12 | 0.09 |
| Mean | 0.09 |

Day 90
WV-49592/
WV-42427 (3 mpk)

| | |
|---|---|
| 13 | 0.11 |
| 14 | 0.14 |
| 15 | 0.14 |
| 16 | 0.10 |
| Mean | 0.12 |

Day 90
WV-49590/
WV-42589 (3 mpk)

| | |
|---|---|
| 17 | 0.20 |
| 18 | 0.18 |
| 19 | 0.21 |
| 20 | 0.23 |
| 21 | 0.22 |
| Mean | 0.21 |

Day 90
WV-49590/
WV-42589 (1.5 mpk)

| | |
|---|---|
| 22 | 0.13 |
| 23 | 0.12 |
| 24 | 0.12 |
| 25 | 0.15 |
| Mean | 0.13 |

Table 31 shows % Ago2 loading of antisense strand relative to miR-122. N=4 or 5.

TABLE 31

| | | | | | Relative HSD/miR- |
|---|---|---|---|---|---|
| | Ct: | Ct: miR- | Ct: | Ct: miR- | 122 normalized |
| | HSD/Ago2 | 122/Ago2 | HSD/IgG | 122/IgG | with WV-42383 |
| WV-42383-1 | 36.61 | 18.86 | 39.33 | 29.5 | 0.162604307 |
| WV-42383-2 | 32.85 | 18.24 | 39.27 | 29.22 | 1.670140758 |
| WV-42383-3 | 33.5 | 17.59 | 39.33 | 29.44 | 0.674238366 |
| WV-42383-4 | 31.75 | 16.97 | 39.13 | 29.78 | 1.493016569 |
| WV-49592-1 | 29.91 | 16.34 | 39.29 | 29.44 | 3.46955462 |
| WV-495920-2 | 31.25 | 16.33 | 38.28 | 29.25 | 1.352694294 |
| WV-49592-3 | 31.4 | 15.69 | 39.02 | 28.26 | 0.784347528 |
| WV-49592-4 | 29.36 | 16.37 | 38.66 | 29.03 | 5.186016614 |
| WV-49590 | 27.04 | 16.44 | 37.73 | 28.64 | 27.20959948 |
| (3 mpk)-1 | | | | | |
| WV-49590 | 28.36 | 15.64 | 38.89 | 28.48 | 6.259039681 |
| (3 mpk)-2 | | | | | |
| WV-49590 | 26.42 | 15.78 | 37.78 | 28.8 | 26.47150658 |
| (3 mpk)-3 | | | | | |
| WV-49590 | 28.92 | 17.07 | 38.19 | 28.76 | 11.42861489 |
| (3 mpk)-4 | | | | | |
| WV-49590 | 25.85 | 15.9 | 35.64 | 28.97 | 42.67404086 |
| (3 mpk)-5 | | | | | |
| WV-49590 | 31.25 | 16.01 | 38.99 | 28.37 | 1.086850212 |
| (1.5 mpk)-1 | | | | | |
| WV-49590 | 28.55 | 15.64 | 37.93 | 28.93 | 5.482182967 |
| (1.5 mpk)-2 | | | | | |
| WV-49590 | 30.86 | 15.29 | 39.48 | 28.31 | 0.866484495 |
| (1.5 mpk)-3 | | | | | |
| WV-49590 | 29.91 | 16.33 | 39.16 | 29.1 | 3.445100299 |
| (1.5 mpk)-4 | | | | | |

While various embodiments have been described and illustrated herein, those of ordinary skill in the art will readily envision a variety of other means and/or structures for performing the functions and/or obtaining the results and/or one or more of the advantages described in the present disclosure, and each of such variations and/or modifications is deemed to be included. More generally, those skilled in the art will readily appreciate that all parameters, dimensions, materials, and configurations described herein are meant to be example and that the actual parameters, dimensions, materials, and/or configurations may depend upon the specific application or applications for which the teachings of the present disclosure is/are used. Those skilled in the art will recognize, or be able to ascertain using no more than routine experimentation, many equivalents to the embodiments of the present disclosure. It is, therefore, to be understood that the foregoing embodiments are presented by way of example only and that, within the scope of the appended claims and equivalents thereto, claimed technologies may be practiced otherwise than as specifically described and claimed. In addition, any combination of two or more features, systems, articles, materials, kits, and/or methods, if such features, systems, articles, materials, kits, and/or methods are not mutually inconsistent, is included within the scope of the present disclosure.

What is claimed is:

1. A double-stranded RNAi (dsRNAi) agent comprising a guide strand and a passenger strand wherein:
a) the guide strand is complementary or substantially complementary to an HSD17B13 target RNA sequence, and comprises:
i. backbone phosphorothioate chiral centers in Sp configuration between the 3′ terminal nucleotide and the penultimate (N-1) nucleotide and as between the penultimate (N-1) nucleotide and the immediately upstream (N-2) nucleotide,
ii. backbone phosphorothioate chiral centers in Rp, Sp, or alternating configurations between the 5′ terminal (+1) nucleotide and the immediately downstream (+2)nucleotide and between the +2 nucleotide and the immediately downstream (+3) nucleotide;
iii. one or more backbone phosphorothioate chiral centers in Rp or Sp configuration upstream of backbone phosphorothioate chiral centers in Sp configuration between the 3′ terminal nucleotide and the penultimate (N-1) nucleotide and as between the penultimate (N-1) nucleotide and the immediately upstream (N-2) nucleotide; and/or
iv. one or more backbone phosphorothioate chiral centers in Rp or Sp configuration between the 5′ terminal (+1) nucleotide and the immediately downstream (+2) nucleotide and between the (+2) nucleotide and the immediately downstream (+3) nucleotide, as well as between one or both of: (a) the (+3) nucleotide and the (+4) nucleotide; and (b) the (+5) nucleotide and the (+6) nucleotide;
b) the guide strand comprises one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
c) the guide strand comprises a 2′ modification, of the 3′ nucleotide of a nucleotide pair linked by an Rp, Sp, or stereorandom non-negatively charged internucleotidic linkage;
d) the passenger strand comprises one or both of:
i. 0-n Rp, Sp, or stereorandom non-negatively charged internucleotidic linkages, where n is about 1 to 49, and
ii. one or more backbone chiral centers in Rp or Sp configuration,
e) each strand of the dsRNAi agent independently has a length of about 15 to about 49 nucleotides;
f) the dsRNAi is capable of directing HSD17B13-specific RNA interference.

2. A chirally controlled oligonucleotide composition comprising double stranded oligonucleotides wherein the guide and passenger strands of the double stranded oligonucleotides are independently characterized by:
a) a common base sequence and length;
b) a common pattern of backbone linkages; and
c) a common pattern of backbone chiral centers;
which composition is chirally controlled in that it is enriched, relative to a substantially racemic preparation of guide strands having the same common base sequence and length, for oligonucleotides having a common pattern of chiral centers; and
a) wherein the guide strands are complementary or substantially complementary to an HSD17B13 target RNA sequence, and comprise:
i. backbone phosphorothioate chiral centers in Sp configuration between the 3′ terminal nucleotide and the penultimate (N-1) nucleotide and as between the penultimate (N-1) nucleotide and the immediately upstream (N-2) nucleotide,
ii. backbone phosphorothioate chiral centers in Rp, Sp, or alternating configurations between the 5′ terminal (+1) nucleotide and the immediately downstream (+2)nucleotide and between the +2 nucleotide and the immediately downstream (+3) nucleotide;
iii. one or more backbone phosphorothioate chiral centers in Rp or Sp configuration upstream of backbone phosphorothioate chiral centers in Sp configuration between the 3′ terminal nucleotide and the penultimate (N-1) nucleotide and as between the penultimate (N-1) nucleotide and the immediately upstream (N-2) nucleotide; and/or
iv. one or more backbone phosphorothioate chiral centers in Rp or Sp configuration between the 5′ terminal (+1) nucleotide and the immediately downstream (+2) nucleotide and between the (+2) nucleotide and the immediately downstream (+3) nucleotide, as well as between one or both of: (a) the (+3) nucleotide and the (+4) nucleotide; and (b) the (+5) nucleotide and the (+6) nucleotide; or
b) the guide strand comprises one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
c) the guide strand comprises a 2′ modification, of the 3′ nucleotide of a nucleotide pair linked by an Rp, Sp, or stereorandom non-negatively charged internucleotidic linkage;
d) the passenger strands comprise one or both of:
i. 0-n Rp, Sp, or stereorandom non-negatively charged internucleotidic linkages, where n is about 1 to 49, and
ii. one or more backbone chiral centers in Rp or Sp configuration,
e) the guide and passenger strands have a length of about 15 to about 49 nucleotides; and
f) the guide and passenger strands are capable of directing HSD17B13-specific RNA interference.

3. The double stranded oligonucleotide of claim 1, wherein the guide strand comprises a 5′ terminal modification selected from: 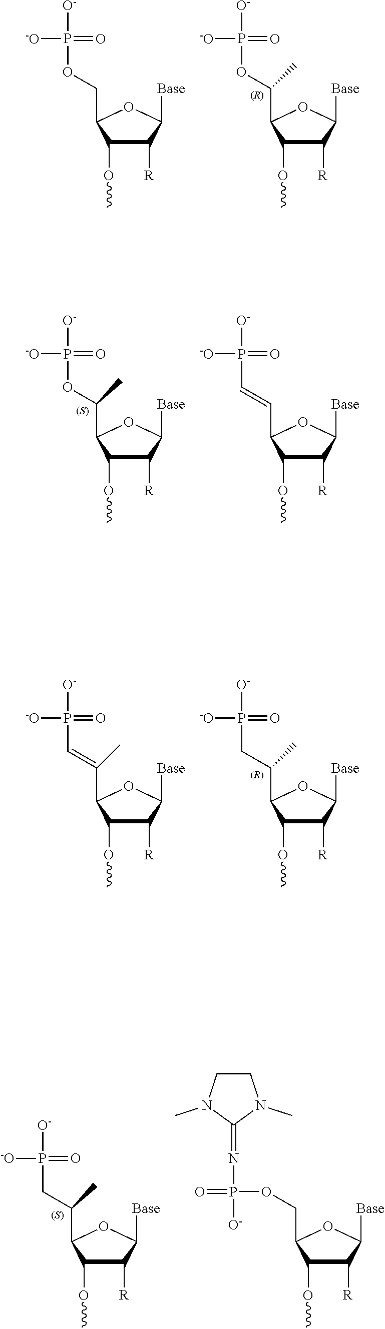 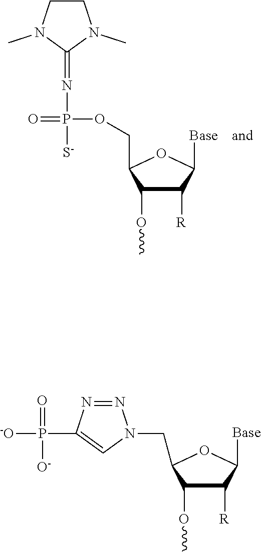
Base: A, C, G, T, U, abasic, and modified nucleobases;
R: H, OH, O-alkyl, F, MOE, LNA bridge to the 4′ position, BNA bridge to the 4′ position.

4. The double stranded oligonucleotide of claim 1, wherein the Rp, Sp, or stereorandom non-negatively charged backbone internucleotidic linkages have neutral charge.

5. The double stranded oligonucleotide or composition of claim 4, wherein the neutral backbone internucleotidic linkages is

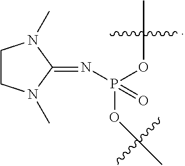 [SMILES: *OP(=O)(N=C1N(C)CCN1C)O*]

6. The composition of claim 2, wherein the guide and passenger strands in the composition that independently share a common base sequence, a common pattern of base modification, a common pattern of sugar modification, and/or a common pattern of internucleotidic linkages are at least 90% of all the guide and passenger strands in the composition.

7. The double stranded oligonucleotide of claim 1, wherein at least 20%, 25%, 30%, 35%, 40%, 45%, 50%, 55%, 60%, 65%, 70%, 75%, 80%, 85%, 90% or 95% of the internucleotidic linkages of the double stranded oligonucleotide are independently chiral internucleotidic linkages.

8. The double stranded oligonucleotide of claim 1, wherein at least 3%, 5%, 10%, 15%, 20%, 25%, 30%, 35%, 40%, 45%, 50%, 55%, 60%, 65%, 70%, 75%, 80%, 85%, 90%, 95%, or 97% of the nucleotidic units of the double stranded oligonucleotide independently comprise a 2′-substitution.

9. The double stranded oligonucleotide of claim 1, wherein the guide strand comprises an HSD17B13 target-binding sequence that is completely complementary to an HSD17B13 target sequence, wherein the HSD17B13 target-binding sequence has a length of at least 10, 11, 12, 13, 14, 15, 16, 17, 18, 19 or 20 bases, wherein each base is optionally substituted adenine, cytosine, guanosine, thymine, or uracil, and wherein the HSD17B13 target sequence comprises one or more allelic sites, wherein an allelic site is a SNP or a mutation.

10. The double stranded oligonucleotide of claim 1, wherein the HSD17B13 target sequence comprises two SNPs.

11. The double stranded oligonucleotide of claim 1, wherein the HSD17B13 target sequence comprises an allelic site and the HSD17B13 target-binding sequence is completely complementary to the HSD17B13 target sequence of a disease-associated allele but not that of an allele less associated with the disease.

12. The double stranded oligonucleotide of claim 1, wherein
the double stranded oligonucleotide comprises a guide strand that binds with a transcript of an HSD17B13 target nucleic acid sequence for which a plurality of alleles exist within a population, each of which contains a specific nucleotide characteristic sequence element that defines the allele relative to other alleles of the same HSD17B13 target nucleic acid sequence,
wherein the base sequence of the guide strand is or comprises a sequence that is complementary to the characteristic sequence element that defines a target allele, and
the guide strand being characterized in that, when it is contacted with a cell comprising transcripts of HSD17B13 target nucleic acid sequence, it shows suppression of transcripts of the target allele, or a protein encoded thereby, at a level that is greater than a level of suppression observed for another allele of the same nucleic acid sequence.

13. The double stranded oligonucleotide of claim 1, comprising:
a guide strand comprising WV-47139 and a passenger strand comprising WV-42589;
a guide strand comprising WV-47159 and a passenger strand comprising WV-42589;
a guide strand comprising WV-49590 and a passenger strand comprising WV-42589;
or
a guide strand comprising WV-49591 and a passenger strand comprising WV-42589

14. A method for reducing level and/or activity of an HSD17B13 transcript or a protein encoded thereby, comprising administering to a cell expressing the HSD17B13 transcript a double stranded oligonucleotide, wherein the double stranded oligonucleotide comprises a guide strand and a passenger strand wherein:
a) the guide strand is complementary or substantially complementary to an HSD17B13 target RNA sequence, and comprises:
i. backbone phosphorothioate chiral centers in Sp configuration between the 3′ terminal nucleotide and the penultimate (N-1) nucleotide and as between the penultimate (N-1) nucleotide and the immediately upstream (N-2) nucleotide,
ii. backbone phosphorothioate chiral centers in Rp, Sp, or alternating configurations between the 5′ terminal (+1) nucleotide and the immediately downstream (+2)nucleotide and between the +2 nucleotide and the immediately downstream (+3) nucleotide;
iii. one or more backbone phosphorothioate chiral centers in Rp or Sp configuration upstream of backbone phosphorothioate chiral centers in Sp configuration between the 3′ terminal nucleotide and the penultimate (N-1) nucleotide and as between the penultimate (N-1) nucleotide and the immediately upstream (N-2) nucleotide; and/or
iv. one or more backbone phosphorothioate chiral centers in Rp or Sp configuration between the 5′ terminal (+1) nucleotide and the immediately downstream (+2) nucleotide and between the (+2) nucleotide and the immediately downstream (+3) nucleotide, as well as between one or both of: (a) the (+3) nucleotide and the (+4) nucleotide; and (b) the (+5) nucleotide and the (+6) nucleotide;
b) the guide strand comprises one or more Rp, Sp, or stereorandom non-negatively charged internucleotidic linkage occurs between any two adjacent nucleotides between the second (+2) nucleotide relative to the 5′ terminal nucleotide of the guide strand and the penultimate 3′ (N-1) nucleotide of the guide strand, where N is the 3′ terminal nucleotide;
c) the guide strand comprises a 2′ modification, of the 3′ nucleotide of a nucleotide pair linked by an Rp, Sp, or stereorandom non-negatively charged internucleotidic linkage;
d) the passenger strand comprises one or both of:
i. 0-n Rp, Sp, or stereorandom non-negatively charged internucleotidic linkages, where n is about 1 to 49, and
ii. one or more backbone chiral centers in Rp or Sp configuration,
e) each strand of the dsRNAi agent independently has a length of about 15 to about 49 nucleotides,
f) the dsRNAi is capable of directing HSD17B13-specific RNA interference;
wherein the guide strand of double stranded oligonucleotide comprises a HSD17B13-binding sequence that is completely complementary to an HSD17B13 target sequence in the transcript.

15. The method of claim 14, wherein the cell is an immune cell, a blood cell, a cardiac cell, a lung cell, an optic cell, a muscle cell, a liver cell, a kidney cell, a brain cell, a cell of the central nervous system, or a cell of the peripheral nervous system.

16. The method of claim 14, for allele specific suppression of an HSD17B13 transcript from a nucleic acid sequence for which a plurality of alleles exist within a population, each of which contains a specific nucleotide characteristic sequence element that defines the allele relative to other alleles of the same HSD17B13 target nucleic acid sequence, the method comprising steps of:
contacting a sample comprising transcripts of the HSD17B13 target nucleic acid sequence with a double stranded oligonucleotide or a composition of any one of the preceding claims,
wherein the guide strand of the double stranded oligonucleotide or composition comprises a HSD17B13-binding sequence that is identical or completely complementary to an HSD17B13 target sequence in the nucleic acid sequence, which HSD17B13 target sequence comprises a characteristic sequence element that defines a target allele, and
wherein when the guide strand of the double stranded oligonucleotide or composition is contacted with a cell comprising transcripts of both the target allele and another allele of the same nucleic acid sequence, transcripts of the target allele are suppressed at a greater level than a level of suppression observed for another allele of the same nucleic acid sequence.

17. The method of claim 14, for allele-specific suppression of an HSD17B13 transcript from a nucleic acid sequence for which a plurality of alleles exist within a population, each of which contains a specific nucleotide characteristic sequence element that defines the allele relative to other alleles of the same HSD17B13 target nucleic acid sequence, the method comprising steps of:
administering to a subject comprising transcripts of the HSD17B13 target nucleic acid sequence with a double stranded oligonucleotide or a composition of any one of the preceding claims,
wherein the guide strand of the double stranded oligonucleotide or composition comprises an HSD17B13-binding sequence that is identical or completely complementary to an HSD17B13 target sequence in the nucleic acid sequence, which HSD17B13 target sequence comprises a characteristic sequence element that defines a target allele, and
wherein when the guide strand of the double stranded oligonucleotide or composition is contacted with a cell comprising transcripts of both the HSD17B13 target allele and another allele of the same nucleic acid sequence, transcripts of the target allele are suppressed at a greater level than a level of suppression observed for another allele of the same nucleic acid sequence.

18. The method of claim 14, wherein when the oligonucleotide or oligonucleotide of the composition is contacted with a cell comprising transcripts of both the HSD17B13 target allele and another allele of the same nucleic acid sequence, it shows suppression of transcripts of the target allele at a level that is:
a) greater than when the composition is absent;
b) greater than a level of suppression observed for another allele of the same nucleic acid sequence; or
c) both greater than when the composition is absent, and greater than a level of suppression observed for another allele of the same nucleic acid sequence.

19. The method of claim 18 wherein the cell is an immune cell, a blood cell, a cardiac cell, a lung cell, an optic cell, a muscle cell, a liver cell, a kidney cell, a brain cell, a cell of the central nervous system, or a cell of the peripheral nervous system.

20. The method of claim 14, wherein suppression of HSD17B13 transcripts of the target allele is at a level that is both greater than when the composition is absent, and greater than a level of suppression observed for another allele of the same nucleic acid sequence.
